(19) United States
(12) Patent Application Publication
(10) Pub. No.: US 2015/0050708 A1
(43) Pub. Date: Feb. 19, 2015

(54) MICROORGANISMS AND METHODS FOR PRODUCING BUTADIENE AND RELATED COMPOUNDS BY FORMATE ASSIMILATION

(71) Applicant: Genomatica, Inc., San Diego, CA (US)

(72) Inventors: Anthony P. BURGARD, Bellefonte, PA (US); Robin E. OSTERHOUT, San Diego, CA (US); Priti PHARKYA, San Diego, CA (US); Stefan ANDRAE, San Diego, CA (US)

(73) Assignee: Genomatica, Inc., San Diego, CA (US)

(21) Appl. No.: 14/213,806

(22) Filed: Mar. 14, 2014

Related U.S. Application Data
(60) Provisional application No. 61/799,255, filed on Mar. 15, 2013.

(51) Int. Cl. C12P 5/02; C12N 15/81; C12P 7/16; C12N 15/70; C12P 7/04; C12P 7/18

(52) U.S. Cl. CPC C12P 5/026; C12P 7/04; C12P 7/18; C12P 7/16; C12N 15/70; C12N 15/81
USPC 435/158; 435/157; 435/160; 435/167; 435/252.2; 435/252.3; 435/252.31; 435/252.32; 435/252.33; 435/252.34; 435/252.35; 435/254.11; 435/254.2; 435/254.21; 435/254.23; 435/254.3; 435/254.9; 568/840; 568/852; 568/909.5; 585/16

(57) ABSTRACT

Provided herein are non-naturally occurring microbial organisms having a formaldehyde fixation pathway and a formate assimilation pathway, which can further include a methanol metabolic pathway, a methanol oxidation pathway, a hydrogenase and/or a carbon monoxide dehydrogenase. These microbial organisms can further include a butadiene, 1,3-butanediol, crotyl alcohol or 3-buten-2-ol pathway. Additionally provided are methods of using such microbial organisms to produce butadiene, 1,3-butanediol, crotyl alcohol or 3-buten-2-ol.

This application claims the benefit of priority of U.S. provisional application Ser. No. 61/799,255, filed Mar. 15, 2013, the entire contents of which is incorporated herein by reference.

### BACKGROUND OF THE INVENTION

The present invention relates generally to metabolic and biosynthetic processes and microbial organisms capable of producing organic compounds, and more specifically to non-naturally occurring microbial organisms having a formate assimilation pathway and an organic compound pathway, such as butadiene, 1,3-butanediol, crotyl alcohol or 3-buten-2-ol.

Over 25 billion pounds of butadiene (1,3-butadiene, BD) are produced annually and is applied in the manufacture of polymers such as synthetic rubbers and ABS resins, and chemicals such as hexamethylenediamine and 1,4-butanediol. For example, butadiene can be reacted with numerous other chemicals, such as other alkenes, e.g. styrene, to manufacture numerous copolymers, e.g. acrylonitrile 1,3-butadiene styrene (ABS), styrene-1,3-butadiene (SBR) rubber, styrene-1,3-butadiene latex. These materials are used in rubber, plastic, insulation, fiberglass, pipes, automobile and boat parts, food containers, and carpet backing. Butadiene is typically produced as a by-product of the steam cracking process for conversion of petroleum feedstocks such as naphtha, liquefied petroleum gas, ethane or natural gas to ethylene and other olefins. The ability to manufacture butadiene from alternative and/or renewable feedstocks would represent a major advance in the quest for more sustainable chemical production processes.

1,3-butanediol (1,3-BDO) is a four carbon diol traditionally produced from acetylene via its hydration. The resulting acetaldehyde is then converted to 3-hydroxybutyraldehyde which is subsequently reduced to form 1,3-BDO. In more recent years, acetylene has been replaced by the less expensive ethylene as a source of acetaldehyde. 1,3-BDO is commonly used as an organic solvent for food flavoring agents. It is also used as a co-monomer for polyurethane and polyester resins and is widely employed as a hypoglycaemic agent. Optically active 1,3-BDO is a useful starting material for the synthesis of biologically active compounds and liquid crystals. A commercial use of 1,3-butanediol is subsequent dehydration to afford 1,3-butadiene (Ichikawa et al., J. of Molecular Catalysis A—Chemical, 256:106-112 (2006); Ichikawa et al., J. of Molecular Catalysis A—Chemical, 231:181-189 (2005)), a 25 billion lb/yr petrochemical used to manufacture synthetic rubbers (e.g., tires), latex, and resins. The reliance on petroleum based feedstocks for either acetylene or ethylene warrants the development of a renewable feedstock based route to 1,3-butanediol and to butadiene.

Crotyl alcohol, also referred to as 2-buten-1-ol, is a valuable chemical intermediate. It serves as a precursor to crotyl halides, esters, and ethers, which in turn are chemical intermediates in the production of monomers, fine chemicals, agricultural chemicals, and pharmaceuticals. Exemplary fine chemical products include sorbic acid, trimethylhydroquinone, crotonic acid and 3-methoxybutanol. Crotyl alcohol is also a precursor to 1,3-butadiene. Crotyl alcohol is currently produced exclusively from petroleum feedstocks. For example Japanese Patent 47-013009 and U.S. Pat. Nos. 3,090,815, 3,090,816, and 3,542,883 describe a method of producing crotyl alcohol by isomerization of 1,2-epoxybutane. The ability to manufacture crotyl alcohol from alternative and/or renewable feedstocks would represent a major advance in the quest for more sustainable chemical production processes.

3-Buten-2-ol (also referenced to as methyl vinyl carbinol (MVC)) is an intermediate that can be used to produce butadiene. There are significant advantages to use of 3-buten-2-ol over 1,3-BDO because there are fewer separation steps and only one dehydration step. 3-Buten-2-ol can also be used as a solvent, a monomer for polymer production, or a precursor to fine chemicals Accordingly, the ability to manufacture 3-buten-2-ol from alternative and/or renewable feedstock would again present a significant advantage for sustainable chemical production processes.

Thus, there exists a need for alternative methods for effectively producing commercial quantities of compounds such as butadiene, 1,3-butanediol, crotyl alcohol or 3-buten-2-ol. The present invention satisfies this need and provides related advantages as well.

### SUMMARY OF INVENTION

In one embodiment, provided herein is a non-naturally occurring microbial organism having a formaldehyde fixation pathway and a formate assimilation pathway, wherein the organism includes at least one exogenous nucleic acid encoding a formaldehyde fixation pathway enzyme disclosed herein that is expressed in a sufficient amount to produce pyruvate, and wherein the organism includes at least one exogenous nucleic acid encoding a formate assimilation pathway enzyme disclosed herein that is expressed in a sufficient amount to produce formaldehyde, pyruvate or acetyl-CoA. In one aspect, the microbial organism can further include a methanol metabolic pathway, a methanol oxidation pathway, a hydrogenase and/or a carbon monoxide dehydrogenase, wherein the organism includes at least one exogenous nucleic acid encoding a methanol metabolic pathway enzyme, a methanol oxidation pathway enzyme, the hydrogenase and/or the carbon monoxide dehydrogenase that is expressed in a sufficient amount to produce formaldehyde or produce or enhance the availability of reducing equivalents. Such organisms of the invention advantageously enhance the production of substrates and/or pathway intermediates for the production of butadiene, 1,3-butanediol, crotyl alcohol or 3-buten-2-ol.

In one embodiment, the organism further includes a butadiene, 1,3-butanediol, crotyl alcohol or 3-buten-2-ol pathway. In certain embodiments, the organism includes at least one exogenous nucleic acid encoding a butadiene, 1,3-butanediol, crotyl alcohol or 3-buten-2-ol pathway enzyme expressed in a sufficient amount to produce butadiene, 1,3-butanediol, crotyl alcohol or 3-buten-2-ol. The invention additionally provides methods of using such microbial organisms to produce butadiene, 1,3-butanediol, crotyl alcohol or 3-buten-2-ol by culturing a non-naturally occurring microbial organism containing a butadiene, 1,3-butanediol, crotyl alcohol or 3-buten-2-ol pathway as described herein under conditions and for a sufficient period of time to produce butadiene, 1,3-butanediol, crotyl alcohol or 3-buten-2-ol.

In one embodiment, provided herein is a non-naturally occurring microbial organism having a butadiene or 3-buten-2-ol pathway. In certain embodiments, the organism includes at least one exogenous nucleic acid encoding a butadiene or 3-buten-2-ol pathway enzyme expressed in a sufficient amount to produce butadiene or 3-buten-2-ol. In certain embodiments, the organism can further include a formaldehyde fixation pathway, a methanol metabolic pathway, a methanol oxidation pathway, a hydrogenase and/or a carbon monoxide dehydrogenase. The invention additionally provides methods of using such microbial organisms to produce butadiene or 3-buten-2-ol by culturing a non-naturally occurring microbial organism containing a butadiene or 3-buten-2-ol pathway as described herein under conditions and for a sufficient period of time to produce butadiene or 3-buten-2-ol.

### BRIEF DESCRIPTION OF THE DRAWINGS

FIG. 1 shows exemplary metabolic pathways enabling the conversion of CO2, formate, formaldehyde, MeOH, glycerol, and glucose to 13BDO and crotyl-alcohol. The enzymatic transformations shown are carried out by the following enzymes: A) methanol dehydrogenase, B) 3-hexulose-6-phosphate synthase, C) 6-phospho-3-hexuloisomerase, D) dihydroxyacetone synthase, E) formate reductase, F) formate ligase, formate transferase, or formate synthetase, G) formyl-CoA reductase, H) formyltetrahydrofolate synthetase, I) methenyltetrahydrofolate cyclohydrolase, J) methylenetetrahydrofolate dehydrogenase, K) spontaneous or formaldehyde-forming enzyme, L) glycine cleavage system, M) serine hydroxymethyltransferase, N) serine deaminase, O) methylenetetrahydrofolate reductase, P) acetyl-CoA synthase, Q) pyruvate formate lyase, R) pyruvate dehydrogenase, pyruvate ferredoxin oxidoreductase, or pyruvate:NADP+ oxidoreductase, S) formate dehydrogenase, T) acetyl-CoA carboxylase, U) acetoacetyl-CoA synthase, V) acetyl-CoA:acetyl-CoA acyltransferase, W) acetoacetyl-CoA reductase (ketone reducing), X) 3-hydroxybutyryl-CoA reductase (aldehyde forming), Y) 3-hydroxybutyraldehyde reductase, Z) 3-hydroxybutyryl-CoA transferase, hydrolase, or synthetase, AA) 3-hydroxybutyrate reductase, AB) 3-hydroxybutyryl-CoA dehydratase (or crotonase), AC) crotonyl-CoA reductase (aldehyde forming), AD) crotonaldehyde reductase, AE) crotonyl-CoA transferase, hydrolase, or synthetase, AF) crotonate reductase, AG) crotyl alcohol dehydratase or chemical dehydration. See abbreviation list below for compound names.

FIG. 2 shows exemplary metabolic pathways enabling the conversion of CO2, formate, formaldehyde, MeOH, glycerol, and glucose to butadiene. The enzymatic transformations shown are carried out by the following enzymes: A) methanol dehydrogenase, B) 3-hexulose-6-phosphate synthase, C) 6-phospho-3-hexuloisomerase, D) dihydroxyacetone synthase, E) formate reductase, F) formate ligase, formate transferase, or formate synthetase, G) formyl-CoA reductase, H) formyltetrahydrofolate synthetase, I) methenyltetrahydrofolate cyclohydrolase, J) methylenetetrahydrofolate dehydrogenase, K) spontaneous or formaldehyde forming enzyme, L) glycine cleavage system, M) serine hydroxymethyltransferase, N) serine deaminase, O) methylenetetrahydrofolate reductase, P) acetyl-CoA synthase, Q) pyruvate formate lyase, R) pyruvate dehydrogenase, pyruvate ferredoxin oxidoreductase, or pyruvate:NADP+ oxidoreductase, S) formate dehydrogenase, T) acetyl-CoA carboxylase, U) acetoacetyl-CoA synthase, V) acetyl-CoA:acetyl-CoA acyltransferase, W) acetoacetyl-CoA reductase (ketone reducing), X) 3-hydroxybutyryl-CoA dehydratase (or crotonase), Y) crotonyl-CoA transferase, hydrolase, or synthetase, AF) crotonate reductase, Z) crotonate reductase, AA) crotonyl-CoA reductase (aldehyde reductase), AB) crotonaldehyde reductase, AC) crotyl alcohol kinase, AD) crotyl-phosphate kinase, AE) butadiene synthase. See abbreviation list below for compound names.

FIG. 3 shows metabolic pathways enabling the extraction of reducing equivalents from methanol, hydrogen, or carbon monoxide. The enzymatic transformations shown are carried out by the following enzymes: A) methanol methyltransferase, B) methylenetetrahydrofolate reductase, C) methylenetetrahydrofolate dehydrogenase, D) methenyltetrahydrofolate cyclohydrolase, E) formyltetrahydrofolate deformylase, F) formyltetrahydrofolate synthetase, G) formate hydrogen lyase, H) hydrogenase, I) formate dehydrogenase, J) methanol dehydrogenase, K) spontaneous or formaldehyde activating enzyme, L) formaldehyde dehydrogenase, M) spontaneous or S-(hydroxymethyl)glutathione synthase, N) Glutathione-Dependent Formaldehyde Dehydrogenase, O) S-formylglutathione hydrolase, P) carbon monoxide dehydrogenase. See abbreviation list below for compound names.

FIG. 4 shows exemplary flux distributions that demonstrate how the maximum theoretical yield of 13BDO from methanol can be increased from 0.167 mol 13BDO/mol methanol (1:6 ratio) to 0.250 mol 13BDO/mol methanol (1:4 ratio) by enabling fixation of formaldehyde with formate reutilization. The upper value of each flux value pair indicates flux distribution for 6.00 mole methanol, and the lower value indicates that for 4 mole methanol when formaldehyde is assimilated with formate reutilization. See abbreviation list below for compound names.

FIG. 5 shows exemplary flux distributions that demonstrate how the maximum theoretical yield of 13BDO from glucose can be increased from 1.00 mol 13BDO/mol glucose (upper value of each flux value pair) to 1.09 mol 13BDO/mol glucose (lower value of each flux value pair) by enabling fixation of formaldehyde with formate reutilization. See abbreviation list below for compound names.

FIG. 6 shows exemplary flux distributions that demonstrate how the maximum theoretical yield of 13BDO from glycerol can be increased from 0.50 mol 13BDO/mol glycerol (upper value of each flux value pair) to 0.64 mol 13BDO/mol glycerol (lower value of each flux value pair) by enabling fixation of formaldehyde with formate reutilization. See abbreviation list below for compound names.

FIG. 7 shows exemplary flux distributions that demonstrate how the maximum theoretical yield of 13BDO from glucose can be increased from 1.00 mol 13BDO/mol glucose (upper value of each flux value pair) to 1.50 mol 13BDO/mol glucose (lower value of each flux value pair) by enabling fixation of formaldehyde with formate reutilization and extraction of reducing equivalents from an external source such as hydrogen. See abbreviation list below for compound names.

FIG. 8 shows exemplary flux distributions that demonstrate how the maximum theoretical yield of 13BDO from glycerol can be increased from 0.50 mol 13BDO/mol glycerol (upper value of each flux value pair) to 0.75 mol 13BDO/mol glycerol (lower value of each flux value pair) by enabling fixation of formaldehyde with formate reutilization and extraction of reducing equivalents from an external source such as hydrogen. See abbreviation list below for compound names.

FIG. 9 shows an exemplary flux distribution that demonstrates how CO2 can be converted to 13BDO using the formaldehyde fixation pathways and an external source of redox such as hydrogen. See abbreviation list below for compound names.

FIG. 10 shows exemplary pathways for formation of 1,3-butanediol and crotyl alcohol from acetyl-CoA. Enzymes are: A. 3-ketoacyl-ACP synthase, B. Acetoacetyl-ACP reductase, C. 3-hydroxybutyryl-ACP dehydratase, D. acetoacetyl-CoA:ACP transferase, E. acetoacetyl-CoA hydrolase, transferase or synthetase, F. acetoacetate reductase (acid reducing), G. 3-oxobutyraldehyde reductase (aldehyde reducing), H. acetoacetyl-ACP thioesterase, I. acetoacetyl-CoA reductase (CoA-dependent, aldehyde forming), J. acetoacetyl-ACP reductase (aldehyde forming), K. acetoacetyl-CoA reductase (alcohol forming), L. 3-hydroxybutyryl-ACP thioesterase, M. 3-hydroxybutyryl-ACP reductase (aldehyde forming), N. 3-hydroxybutyryl-CoA reductase (aldehyde forming), O. 3-hydroxybutyryl-CoA reductase (alcohol forming), P. acetoacetyl-CoA reductase (ketone reducing), Q. acetoacetate reductase (ketone reducing), R. 3-oxobutyraldehyde reductase (ketone reducing), S. 4-hydroxy-2-butanone reductase, T. crotonyl-ACP thioesterase, U. crotonyl-ACP reductase (aldehyde forming), V. crotonyl-CoA reductase (aldehyde forming), W. crotonyl-CoA (alcohol forming), X. 3-hydroxybutyryl-CoA:ACP transferase, Y. 3-hydroxybutyryl-CoA hydrolase, transferase or synthetase, Z. 3-hydroxybutyrate reductase, AA. 3-hydroxybutyraldehyde reductase, AB. 3-hydroxybutyryl-CoA dehydratase, AC. 3-hydroxybutyrate dehydratase, AD. 3-hydroxybutyraldehyde dehydratase, AE. crotonyl-CoA:ACP transferase, AF. crotonyl-CoA hydrolase, transferase or synthetase, AG. crotonate reductase, AH. crotonaldehyde reductase, AS. acetoacetyl-CoA synthase, AT. acetyl-CoA:acetyl-CoA acyltransferase, AU. 4-hydroxybutyryl-CoA dehydratase. ACP is acyl carrier protein.

FIG. 11 shows pathways for conversion of crotyl alcohol to butadiene. Enzymes are: A. crotyl alcohol kinase, B. 2-butenyl-4-phosphate kinase, C. butadiene synthase, D. crotyl alcohol diphosphokinase, E. crotyl alcohol dehydratase or chemical dehydration.

FIG. 12 shows an exemplary pathway for production of butadiene from malonyl-CoA plus acetyl-CoA. Enzymes for transformation of the identified substrates to products include: A. malonyl-CoA:acetyl-CoA acyltransferase, B. 3-oxoglutaryl-CoA reductase (ketone-reducing), C. 3-hydroxyglutaryl-CoA reductase (aldehyde forming), D. 3-hydroxy-5-oxopentanoate reductase, E. 3,5-dihydroxypentanoate kinase, F. 3H5PP kinase, G. 3H5PDP decarboxylase, H. butenyl 4-diphosphate isomerase, I. butadiene synthase, J. 3-hydroxyglutaryl-CoA reductase (alcohol forming), K. 3-oxoglutaryl-CoA reductase (aldehyde forming), L. 3,5-dioxopentanoate reductase (ketone reducing), M. 3,5-dioxopentanoate reductase (aldehyde reducing), N. 5-hydroxy-3-oxopentanoate reductase, O. 3-oxo-glutaryl-CoA reductase (CoA reducing and alcohol forming). Compound abbreviations include: 3H5PP=3-Hydroxy-5-phosphonatooxypentanoate and 3H5PDP=3-Hydroxy-5-[hydroxy(phosphonooxy)phosphoryl]oxy pentanoate.

FIG. 13. Pathway for converting 2-butanol to 3-buten-2-ol. Step A is catalyzed by 2-butanol desaturase. Step B is catalyzed by 3-buten-2-ol dehydratase or chemical dehydration.

FIG. 14. Pathway for converting pyruvate to 2-butanol. Enzymes are A. acetolactate synthase, B. acetolactate decarboxylase, C. butanediol dehydrogenase, D. butanediol dehydratase, E. butanol dehydrogenase.

FIG. 15. Pathway for converting 1,3-butanediol to 3-buten-2-ol and/or butadiene. Enzymes are A. 1,3-butanediol kinase, B. 3-hydroxybutyrylphosphate kinase, C. 3-hydroxybutyryldiphosphate lyase, D. 1,3-butanediol diphosphokinase, E. 1,3-butanediol dehydratase, F. 3-hydroxybutyrylphosphate lyase, G. 3-buten-2-ol dehydratase or chemical reaction.

FIG. 16. Pathway for converting acrylyl-CoA to 3-buten-2-ol or butadiene. Enzymes are A. 3-oxopent-4-enoyl-CoA thiolase, B. 3-oxopent-4-enoyl-CoA hydrolase, synthetase or transferase, C. 3-oxopent-4-enoate decarboxylase or spontaneous, D. 3-buten-2-one reductase and E. 3-buten-2-ol dehydratase or chemical dehydration.

FIG. 17. Pathways for converting lactoyl-CoA to 3-buten-2-ol and/or butadiene. Enzymes are A. 3-Oxo-4-hydroxypentanoyl-CoA thiolase, B. 3-oxo-4-hydroxypentanoyl-CoA transferase, synthetase or hydrolase, C. 3-oxo-4-hydroxypentanoate reductase, D. 3,4-dihydroxypentanoate decarboxylase, E. 3-oxo-4-hydroxypentanoyl-CoA reductase, F. 3,4-dihydroxypentanoyl-CoA transferase, synthetase or hydrolase, G. 3-buten-2-ol dehydratase or chemical dehydration, H. 3,4-dihydroxypentanoate dehydratase, I. 4-oxopentanoate reductase, J. 4-hyd4-oxoperoxypentanoate decarboxylase.

FIG. 18. Pathways for converting succinyl-CoA to 3-buten-2-ol and/or butadiene. Enzymes are A. 3-oxoadipyl-CoA thiolase, B. 3-oxoadipyl-CoA transferase, synthetase or hydrolase, C. 3-oxoadipate decarboxylase or spontaneous reaction (non-enzymatic), D. 4-oxopentanoate reductase, E. 4-hydroxypentanoate decarboxylase, F. 3-buten-2-ol dehydratase or chemical dehydration.

### DETAILED DESCRIPTION OF THE INVENTION

The following is a list of abbreviations and their corresponding compound or composition names. These abbreviations, which are used throughout the disclosure and the figures. It is understood that one of ordinary skill in the art can readily identify these compounds/compostions by such nomenclature. MeOH or MEOH=methanol; Fald=formaldehyde; GLC=glucose; G6P=glucose-6-phosphate; H6P=hexulose-6-phosphate; F6P=fructose-6-phosphate; FDP=fructose diphosphate or fructose-1,6-diphosphate; DHA=dihydroxyacetone; DHAP=dihydroxyacetone phosphate; G3P=and glyceraldehyde-3-phosphate; PYR=pyruvate; ACCOA=acetyl-CoA; AACOA=acetoacetyl-CoA; MALCOA=malonyl-CoA; FTHF=formyltetrahydrofolate; THF=tetrahydrofolate; E4P=erythrose-4-phosphate: Xu5P=xyulose-5-phosphate; Ru5P=ribulose-5-phosphate; S7P=sedoheptulose-7-phosphate: R5P=ribose-5-phosphate; 3HBCOA=3-hydroxybutryl-CoA; 3HB=3-hydroxybutyrate; 3HBALD=3-hydroxyburylaldehyde-CoA; 13BDO=1,3-butanediol; CROTCOA=crotonyl-CoA or crotyl-CoA; CROT=crotonate; CROTALD=crotonaldehyde; CROTALC=crotyl alcohol or crotonyl alcohol; BD=butadiene; CROT-Pi=crotyl phosphate or 2-butenyl-4-diphosphate; CROT-PPi=crotyl diphosphate or 2-butenyl-4-diphosphate; TCA=tricarboxylic acid

It is also understood that association of multiple steps in a pathway can be indicated by linking their step identifiers with or without spaces or punctuation; for example, the following are equivalent to describe the 4-step pathway comprising Step W, Step X, Step Y and Step Z: steps WXYZ or W,X,Y,Z or W;X;Y;Z or W-X-Y-Z. One of ordinary skill can readily distinguish a single step designator of “AA” or “AB” or “AD” from a multiple step pathway description based on context and use in the description and figures herein.

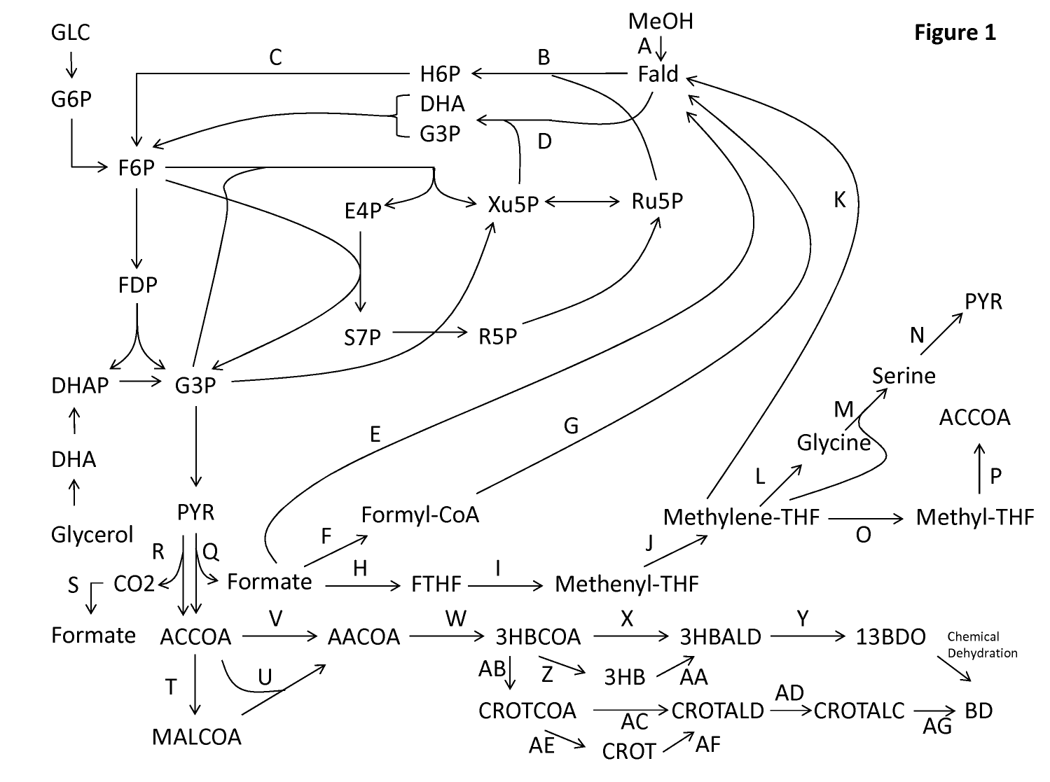

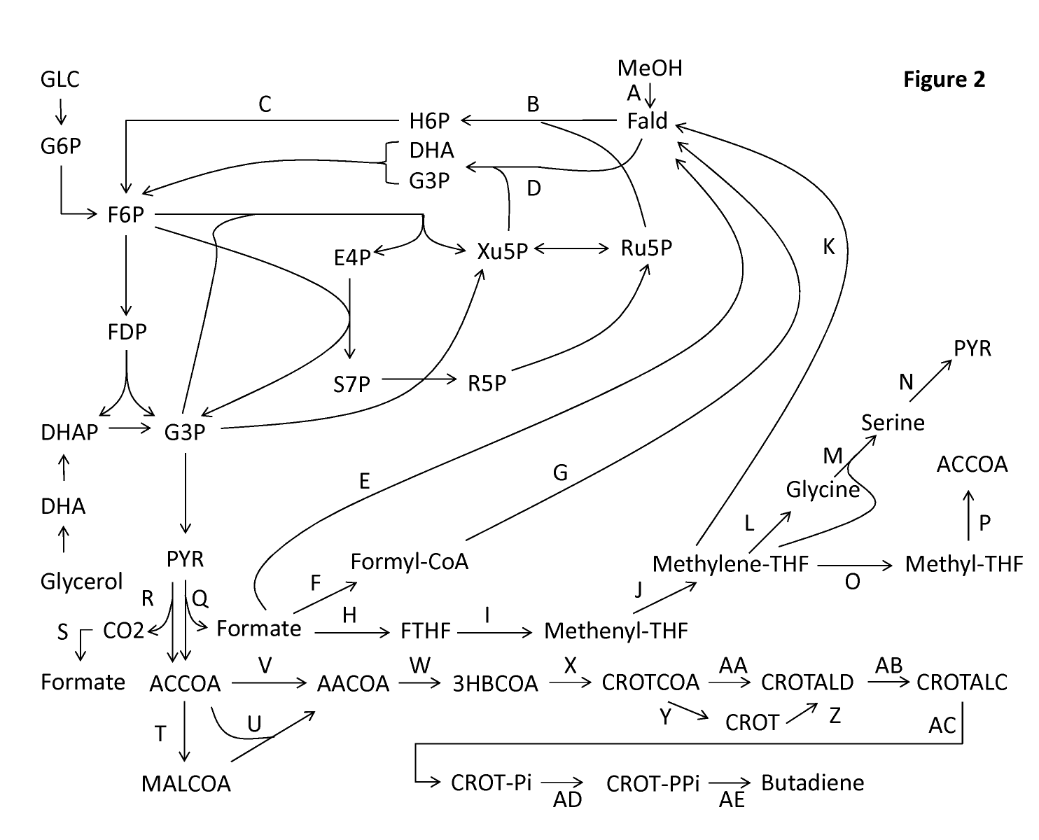

Methanol is a relatively inexpensive organic feedstock that can be used as a redox, energy, and carbon source for the production of chemicals such as butadiene, 1,3-butanediol, crotyl alcohol, and 3-buten-2-ol, and their intermediates, by employing one or more methanol metabolic enzymes as described herein, for example as shown in FIGS. 1, 2, and 3. Methanol can enter central metabolism in most production hosts by employing methanol dehydrogenase (FIG. 1, step A) along with a pathway for formaldehyde assimilation. One exemplary formaldehyde assimilation pathway that can utilize formaldehyde produced from the oxidation of methanol is shown in FIG. 1, which involves condensation of formaldehyde and D-ribulose-5-phosphate to form hexulose-6-phosphate (H6P) by hexulose-6-phosphate synthase (FIG. 1, step B). The enzyme can use Mg²⁺ or Mn²⁺ for maximal activity, although other metal ions are useful, and even non-metal-ion-dependent mechanisms are contemplated. H6P is converted into fructose-6-phosphate by 6-phospho-3-hexuloisomerase (FIG. 1, step C). Another exemplary pathway that involves the detoxification and assimilation of formaldehyde produced from the oxidation of methanol proceeds through dihydroxyacetone. Dihydroxyacetone synthase (FIG. 1, step D) is a transketolase that first transfers a glycoaldehyde group from xylulose-5-phosphate to formaldehyde, resulting in the formation of dihydroxyacetone (DHA) and glyceraldehyde-3-phosphate (G3P), which is an intermediate in glycolysis. The DHA obtained from DHA synthase can be then further phosphorylated to form DHA phosphate by a DHA kinase. DHAP can be assimilated into glycolysis, e.g. via isomerization to G3P, and several other pathways. Alternatively, DHA and G3P can be converted by fructose-6-phosphate aldolase to form fructose-6-phosphate (F6P). The above also applies to FIG. 2.

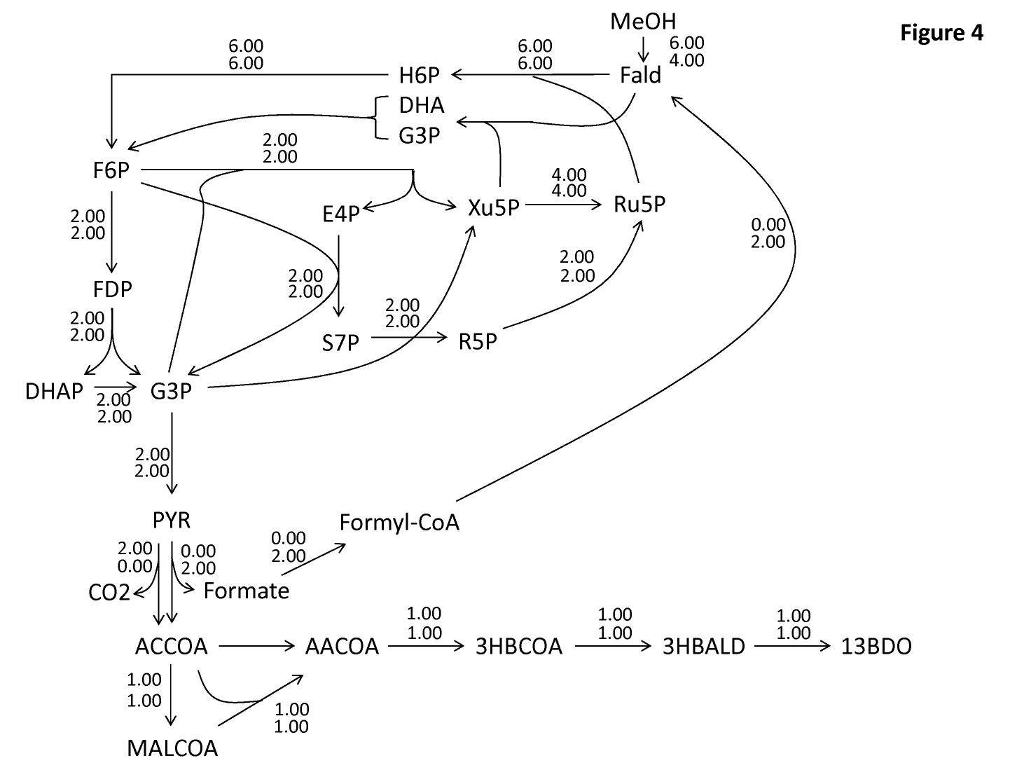

By combining the pathways for methanol oxidation (FIG. 1, step A) and formaldehyde fixation (FIG. 1, Steps B and C or Step D), molar yields of 0.167 mol product/mol methanol can be achieved for 1,3-BDO, crotyl alcohol, and butadiene, and their intermediates. The same applies to FIG. 2 and when methanol oxidation and formaldehyde fixation pathways are combined with other product synthesis pathways for 13BDO, crotyl alcohol and butadiene such as those described herein. For example, FIG. 4 shows an exemplary flux distribution that will lead to a 0.167 mol 1,3-BDO/mol MeOH yield (see the upper flux value of each flux value pair; 1:6 mole ratio 13BDO:MeOH). The following maximum theoretical yield stoichiometries for 1,3-BDO, crotyl alcohol, and butadiene are thus made possible by combining the steps for methanol oxidation, formaldehyde fixation, and product synthesis.

6CH₄O+3.5O₂→C₄H₁₀O₂+7H₂O+2CO₂ (1,3-BDO on MeOH)

6CH₄O+3.5O₂→C₄H₈O+8H₂O+2CO₂ (Crotyl Alcohol on MeOH)

6CH₄O+3.5O₂→C₄H₆+9H₂O+2CO₂ (Butadiene on MeOH)

The yield on several substrates, including methanol, can be further increased by capturing some of the carbon lost from the conversion of pathway intermediates, e.g. pyruvate to acetyl-CoA, using one of the formate reutilization pathways shown in FIG. 1. For example, the CO₂ generated by conversion of pyruvate to acetyl-CoA (FIG. 1, step R) can be converted to formate via formate dehydrogenase (FIG. 1, step S). Alternatively, pyruvate formate lyase, which forms formate directly instead of CO₂, can be used to convert pyruvate to acetyl-CoA (FIG. 1, step Q). Formate can be converted to formaldehyde by using: 1) formate reductase (FIG. 1, step E), 2) a formyl-CoA synthetase, transferase, or ligase along with formyl-CoA reductase (FIG. 1, steps F-G), or 3) formyltetrahydrofolate synthetase, methenyltetrahydrofolate cyclohydrolase, methylenetetrahydrofolate dehydrogenase, and formaldehyde-forming enzyme (FIG. 1, steps H-I-J-K). Conversion of methylene-THF to formaldehyde alternatively will occur spontaneously. Alternatively, formate can be reutilized by converting it to pyruvate or acetyl-CoA using FIG. 1, steps H-I-J-L-M-N or FIG. 1, steps H-I-J-O-P, respectively. Formate reutilization is also useful when formate is an external carbon source. For example, formate can be obtained from organocatalytic, electrochemical, or photoelectrochemical conversion of CO2 to formate. An alternative source of methanol for use in the present methods is organocatalytic, electrochemical, or photoelectrochemical conversion of CO2 to methanol, The above applies to FIG. 2.

By combining the pathways for methanol oxidation (FIG. 1, step A), formaldehyde fixation (FIG. 1, Steps B and C or Step D), and formate reutilization, molar yields as high as 0.250 mol product/mol methanol can be achieved for 1,3-BDO, crotyl alcohol, and butadiene. The same applies to FIG. 2 and when methanol oxidation, formaldehyde fixation and formate reutilization pathways are combined with other product synthesis pathways for 13BDO, crotyl alcohol and butadiene such as those described herein. For example, FIG. 4 shows an exemplary flux distribution that will lead to a 0.250 mol 1,3-BDO/mol MeOH yield (see the lower flux value of each flux value pair; 1:4 mole ratio 13BDO:MeOH). The following maximum theoretical yield stoichiometries for 1,3-BDO, crotyl alcohol, and butadiene are thus made possible by combining the steps for methanol oxidation, formaldehyde fixation, formate reutilization, and product synthesis.

4CH4O+0.5O2→C4H10O2+3H2O (1,3-BDO on MeOH)

4CH4O+0.5O2→C4H8O+4H2O (Crotyl Alcohol on MeOH)

4CH4O+0.5O2→C4H6+5H2O (Butadiene on MeOH)

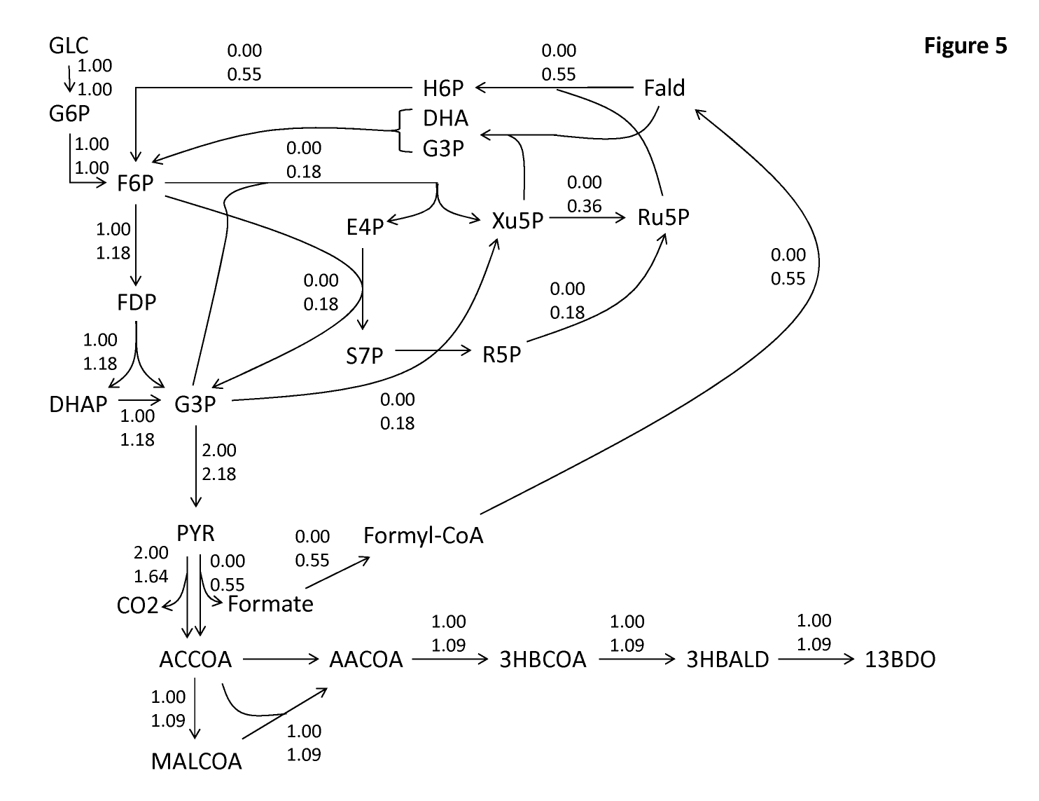

By combining pathways for formaldehyde fixation and formate reutilization, yield increases on additional substrates are also available including but not limited to glucose, glycerol, sucrose, fructose, xylose, arabinose and galactose. For example, FIG. 5 shows exemplary flux distributions that demonstrate how the maximum theoretical yield of 1,3-BDO from glucose can be increased from 1.00 mol 1,3-BDO/mol glucose to 1.09 mol 1,3-BDO/mol glucose (compare the upper and lower flux value of each flux value pair) by enabling fixation of formaldehyde from generation and utilization of formate. The following maximum theoretical yield stoichiometries for 1,3-BDO, crotyl alcohol, and butadiene on glucose are thus made possible by combining the steps for formaldehyde fixation, formate reutilization, and product synthesis.

11C₆H₁₂O₆→12C₄H₁₀O₂+6H₂O+18CO₂ (1,3-BDO on glucose)

11C₆H₁₂O₆→12C₄H₈O+18H₂O+18CO₂ (Crotyl Alcohol on glucose)

11C₆H₁₂O₆→12C₄H₆+30H₂O+18CO₂ (Butadiene on glucose)

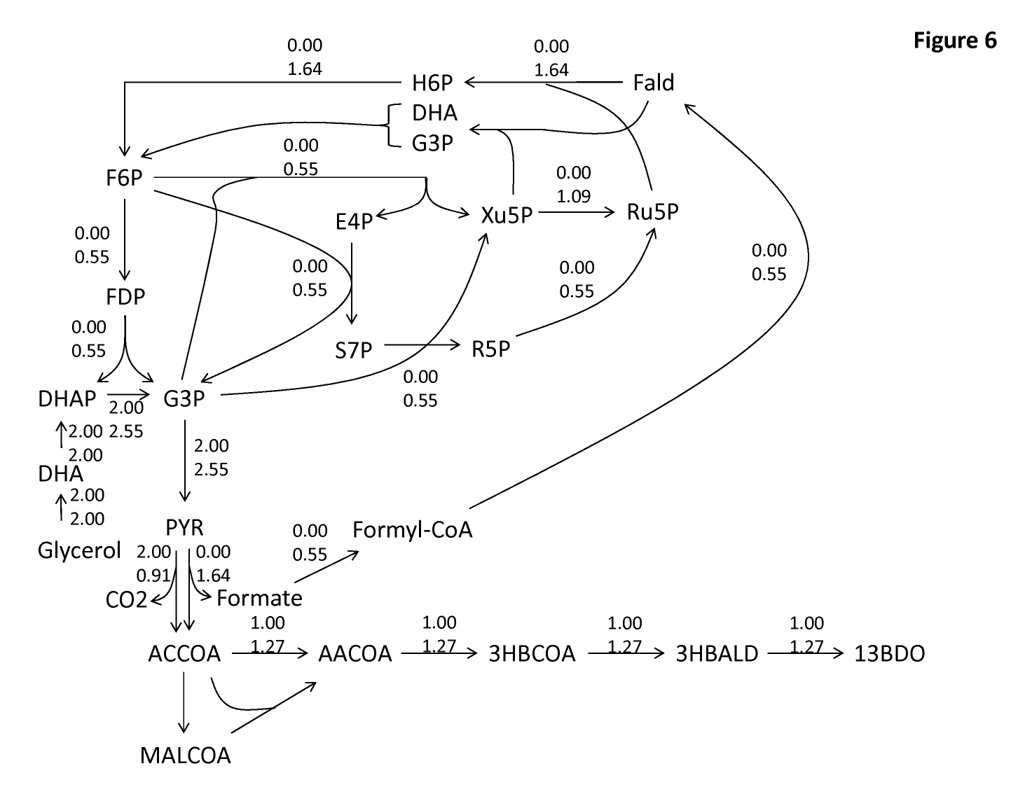

Similarly, FIG. 6 shows exemplary flux distributions that demonstrate how the maximum theoretical yield of 1,3-BDO from glycerol can be increased from 0.50 mol 1,3-BDO/mol glycerol to 0.64 mol 1,3-BDO/mol glycerol (compare the upper and lower flux value of each flux value pair) by enabling fixation of formaldehyde from generation and utilization of formate. The following maximum theoretical yield stoichiometries for 1,3-BDO, crotyl alcohol, and butadiene on glycerol are thus made possible by combining the steps for formaldehyde fixation, formate reutilization, and product synthesis.

11C₃H₈O₃→7C₄H₁₀O₂+9H₂O+5CO₂ (1,3-BDO on glycerol)

11C₃H₈O₃→7C₄H₈O+16H₂O+5CO₂ (Crotyl Alcohol on glycerol)

11C₃H₈O₃→7C₄H₆+23H₂O+5CO₂ (Butadiene on glycerol)

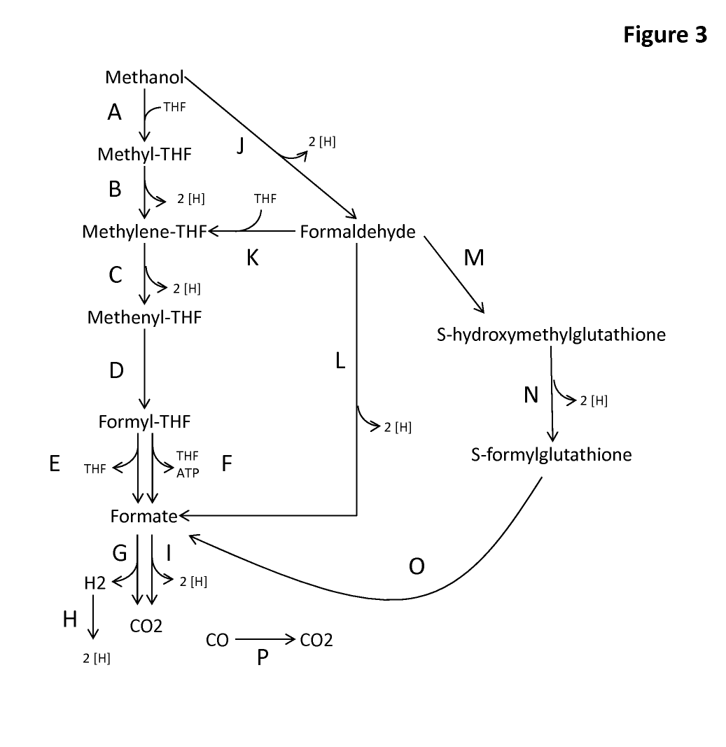

In numerous engineered pathways, product yields based on carbohydrate feedstock are hampered by insufficient reducing equivalents or by loss of reducing equivalents to byproducts. Methanol is a relatively inexpensive organic feedstock that can be used to generate reducing equivalents by employing one or more methanol metabolic enzymes as shown in FIG. 3. Reducing equivalents can also be extracted from hydrogen and carbon monoxide by employing hydrogenase and carbon monoxide dehydrogenase enzymes, respectively, as shown in FIG. 3. The reducing equivalents are then passed to acceptors such as oxidized ferredoxins, oxidized quinones, oxidized cytochromes, NAD(P)+, water, or hydrogen peroxide to form reduced ferredoxin, reduced quinones, reduced cytochromes, NAD(P)H, H₂, or water, respectively. Reduced ferredoxin, reduced quinones and NAD(P)H are particularly useful as they can serve as redox carriers for various Wood-Ljungdahl pathway, reductive TCA cycle, or product pathway enzymes.

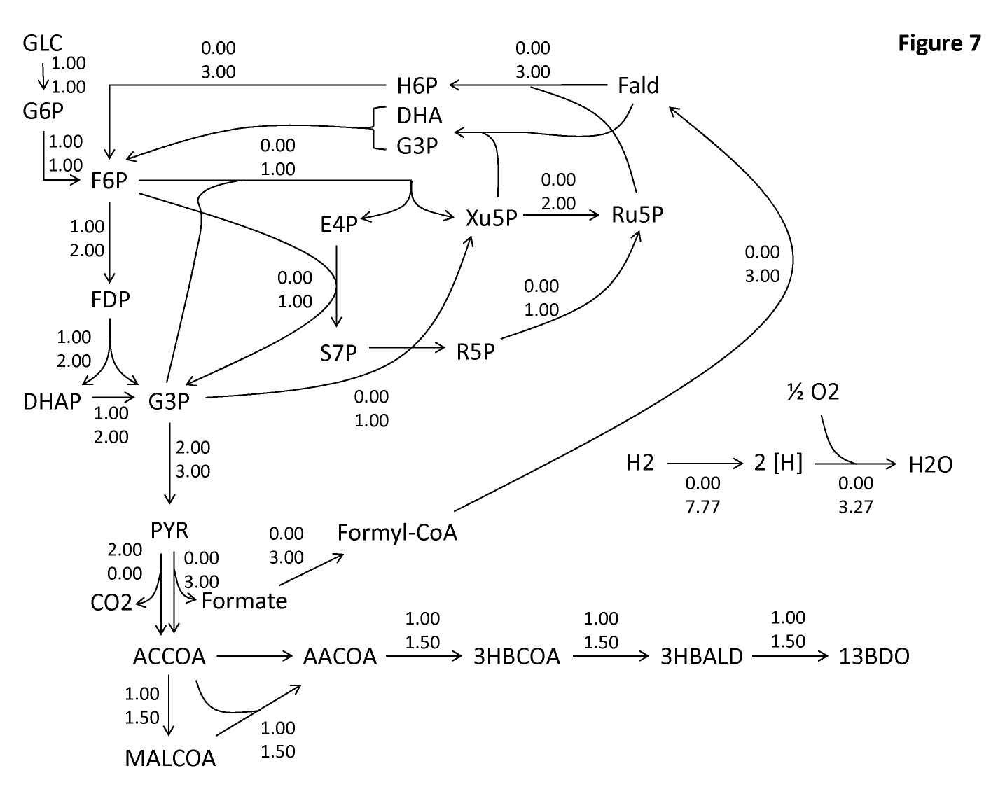

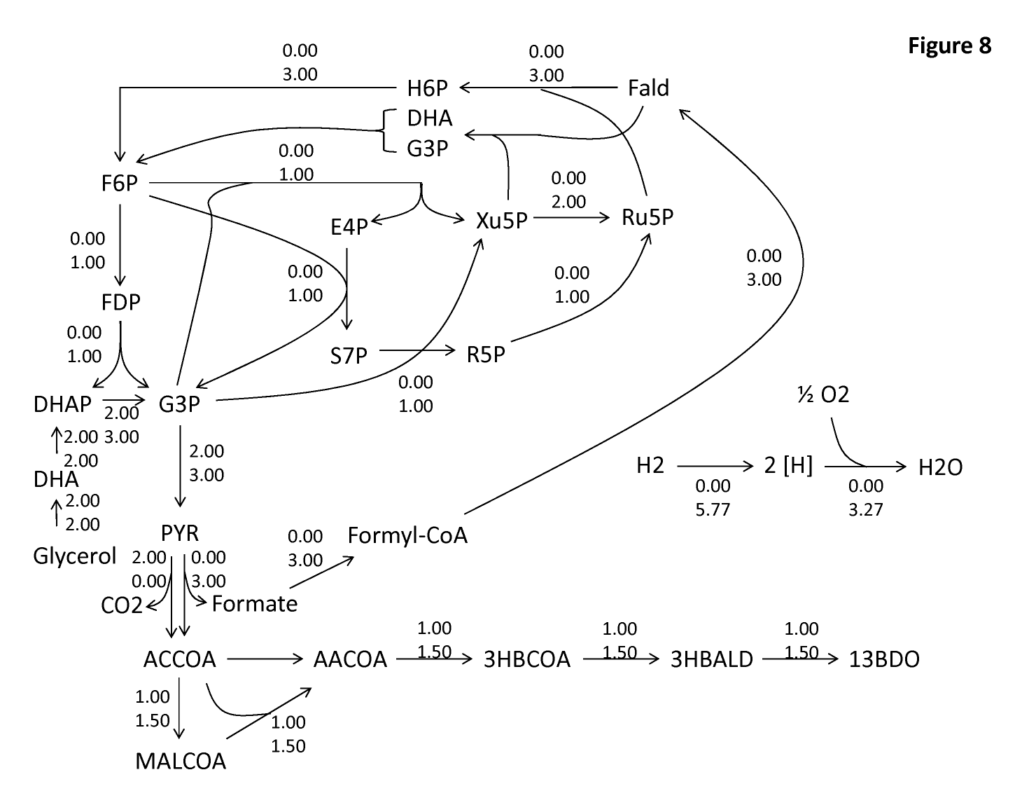

The reducing equivalents produced by the metabolism of methanol, hydrogen, and carbon monoxide can be used to power several 1,3-BDO, crotyl alcohol, and butadiene production pathways. For example, FIG. 7 and FIG. 8 show exemplary flux distributions that demonstrate how the maximum theoretical yield of 1,3-BDO from glucose and glycerol, respectively, can be increased by enabling fixation of formaldehyde, formate reutilization, and extraction of reducing equivalents from an external source such as hydrogen. In fact, by combining pathways for formaldehyde fixation, formate reutilization, reducing equivalent extraction, and product synthesis, the following maximum theoretical yield stoichiometries for 1,3-BDO, crotyl alcohol, and butadiene on glucose and glycerol are made possible.

C₆H₁₂O₆+4.5H₂→1.5C₄H₁₀O₂+3H₂O (1,3-BDO on glucose+external redox)

C₆H₁₂O₆+4.5H₂→1.5C₄H₈O+4.5H₂O (Crotyl Alcohol on glucose+external redox)

C₆H₁₂O₆+4.5H₂→1.5C₄H₆+6H₂O (Butadiene on glucose+external redox)

C₃H₈O₃+1.25H₂→0.75C₄H₁₀O₂+1.5H₂O (1,3-BDO on glycerol+external redox)

C₃H₈O₃+1.25H₂→0.75C₄H₈O+2.25H₂O (Crotyl Alcohol on glycerol+external redox)

C₃H₈O₃+1.25H₂→0.75C₄H₆+3H₂O (Butadiene on glycerol+external redox)

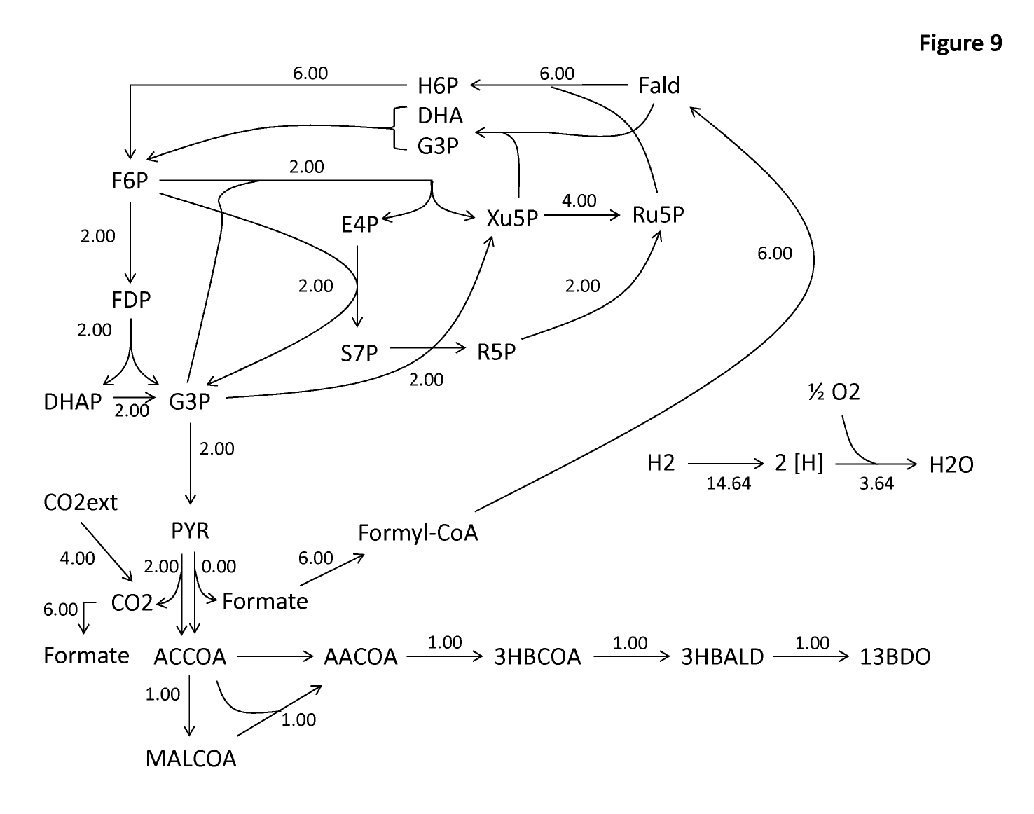

In most instances, achieving such maximum yield stoichiometries may require some oxidation of reducing equivalents (e.g., H₂+½O₂→H₂O, CO+½O₂→CO₂, CH₄O+1.5O₂→CO₂+2H₂O, C₆H₁₂O₆+6O₂→6CO₂6H₂O) to provide sufficient energy for the substrate to product pathways to operate. Nevertheless, if sufficient reducing equivalents are available, enabling pathways for fixation of formaldehyde, formate reutilization, extraction of reducing equivalents, and product synthesis can even lead to production of 1,3-BDO, crotyl alcohol, and butadiene, and their intermediates, directly from CO₂ as demonstrated in FIG. 9.

Pathways identified herein, and particularly pathways exemplified in specific combinations presented herein, are superior over other pathways based in part on the applicant's ranking of pathways based on attributes including maximum theoretical BDO yield, maximal carbon flux, maximal production of reducing equivalents, minimal production of CO2, pathway length, number of non-native steps, thermodynamic feasibility, number of enzymes active on pathway substrates or structurally similar substrates, and having steps with currently characterized enzymes, and furthermore, the latter pathways are even more favored by having in addition at least the fewest number of non-native steps required, the most enzymes known active on pathway substrates or structurally similar substrates, and the fewest total number of steps from central metabolism.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a butadiene, 1,3-butanediol, crotyl alcohol or 3-buten-2-ol biosynthetic pathway.

A metabolic modification refers to a biochemical reaction that is altered from its naturally occurring state. Therefore, non-naturally occurring microorganisms can have genetic modifications to nucleic acids encoding metabolic polypeptides, or functional fragments thereof. Exemplary metabolic modifications are disclosed herein.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “ACP” or “acyl carrier protein” refers to any of the relatively small acidic proteins that are associated with the fatty acid synthase system of many organisms, from bacteria to plants. ACPs can contain one 4′-phosphopantetheine prosthetic group bound covalently by a phosphate ester bond to the hydroxyl group of a serine residue. The sulfhydryl group of the 4′-phosphopantetheine moiety serves as an anchor to which acyl intermediates are (thio)esterified during fatty-acid synthesis. An example of an ACP is Escherichia coli ACP, a separate single protein, containing 77 amino-acid residues (8.85 kDa), wherein the phosphopantetheine group is linked to serine 36.

As used herein, the term “butadiene,” having the molecular formula C₄H₆ and a molecular mass of 54.09 g/mol (see FIGS. 1, 5, 6 and 12) (IUPAC name Buta-1,3-diene) is used interchangeably throughout with 1,3-butadiene, biethylene, erythrene, divinyl, vinylethylene. Butadiene is a colorless, non corrosive liquefied gas with a mild aromatic or gasoline-like odor. Butadiene is both explosive and flammable because of its low flash point.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood, as disclosed herein, that such more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

The non-naturally occurring microbial organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having butadiene, 1,3-butanediol, crotyl alcohol or 3-buten-2-ol biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan., 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep., 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In certain embodiments, provided herein is a non-naturally occurring microbial organism having a formaldehyde fixation pathway and a formate assimilation pathway. In certain embodiments, the organism comprises at least one exogenous nucleic acid encoding a formaldehyde fixation pathway enzyme expressed in a sufficient amount to produce pyruvate, wherein said formaldehyde fixation pathway comprises 1B, 1C, or 1D or any combination thereof, wherein 1B is a 3-hexulose-6-phosphate synthase, wherein 1C is a 6-phospho-3-hexuloisomerase, wherein 1D is a dihydroxyacetone synthase. In certain embodiments, the organism comprises at least one exogenous nucleic acid encoding a formate assimilation pathway enzyme expressed in a sufficient amount to produce formaldehyde, pyruvate, or acetyl-CoA, wherein said formate assimilation pathway comprises 1E, 1F, 1G, 1H, 1I, 1J, 1K, 1L, 1M, 1N, 1O, or 1P or any combination thereof, wherein 1E is a formate reductase, 1F is a formate ligase, a formate transferase, or a formate synthetase, wherein 1G is a formyl-CoA reductase, wherein 1H is a formyltetrahydrofolate synthetase, wherein 1I is a methenyltetrahydrofolate cyclohydrolase, wherein 1J is a methylenetetrahydrofolate dehydrogenase, wherein 1K is a formaldehyde-forming enzyme or spontaneous, wherein 1L is a glycine cleavage system, wherein 1M is a serine hydroxymethyltransferase, wherein 1N is a serine deaminase, wherein 1O is a methylenetetrahydrofolate reductase, wherein 1P is an acetyl-CoA synthase.

In one embodiment, the formaldehyde fixation pathway comprises 1B. In one embodiment, the formaldehyde fixation pathway comprises 1C. In one embodiment, the formaldehyde fixation pathway comprises 1D. In one embodiment, the formate assimilation pathways comprises 1E. In one embodiment, the formate assimilation pathways comprises 1F, 1G. In one embodiment, the formate assimilation pathways comprises 1H. In one embodiment, the formate assimilation pathways comprises 1I. In one embodiment, the formate assimilation pathways comprises 1J. In one embodiment, the formate assimilation pathways comprises 1K. In one embodiment, the formate assimilation pathways comprises 1L. In one embodiment, the formate assimilation pathways comprises 1M. In one embodiment, the formate assimilation pathways comprises 1N. In one embodiment, the formate assimilation pathways comprises 1O. In one embodiment, the formate assimilation pathways comprises 1P. Any combination of two, three, four, five, six, seven, eight, nine, ten, eleven, twelve, thirteen, fourteen, fifteen pathway enzymes of 1B, 1C, 1D, 1E, 1F, 1G, 1H, 1I, 1J, 1K, 1L, 1M, 1N, 1O, or 1P is also contemplated.

In one aspect, provided herein is a non-naturally occurring microbial organism having a formaldehyde fixation pathway and a formate assimilation pathway, wherein said organism comprises at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase, wherein said organism comprises at least one exogenous nucleic acid encoding a formate assimilation pathway enzyme expressed in a sufficient amount to produce formaldehyde, pyruvate, or acetyl-CoA, wherein said formate assimilation pathway comprises a pathway selected from: (3) 1E; (4) 1F, and 1G; (5) 1H, 1I, 1J, and 1K; (6) 1H, 1I, 1J, 1L, 1M, and 1N; (7) 1E, 1H, 1I, 1J, 1L, 1M, and 1N; (8) 1F, 1G, 1H, 1I, 1J, 1L, 1M, and 1N; (9) 1K, 1H, 1I, 1J, 1L, 1M, and 1N; and (10) 1H, 1I, 1J, 1O, and 1P.

In certain embodiments, the formaldehyde fixation pathway comprises 1B and 1C. In certain embodiments, the formaldehyde fixation pathway comprises 1B and 1C, and the formate assimilation pathway comprises 1E. In certain embodiments, the formaldehyde fixation pathway comprises 1B and 1C, and the formate assimilation pathway comprises 1F, and 1G. In certain embodiments, the formaldehyde fixation pathway comprises 1B and 1C, and the formate assimilation pathway comprises 1H, 1I, 1J, and 1K. In certain embodiments, the formaldehyde fixation pathway comprises 1B and 1C, and the formate assimilation pathway comprises 1H, 1I, 1J, 1L, 1M, and 1N. In certain embodiments, the formaldehyde fixation pathway comprises 1B and 1C, and the formate assimilation pathway comprises 1E, 1H, 1I, 1J, 1L, 1M, and 1N. In certain embodiments, the formaldehyde fixation pathway comprises 1B and 1C, and the formate assimilation pathway comprises 1F, 1G, 1H, 1I, 1J, 1L, 1M, and 1N. In certain embodiments, the formaldehyde fixation pathway comprises 1B and 1C, and the formate assimilation pathway comprises 1K, 1H, 1I, 1J, 1L, 1M, and 1N. In certain embodiments, the formaldehyde fixation pathway comprises 1B and 1C, and the formate assimilation pathway comprises 1H, 1I, 1J, 1O, and 1P.

In certain embodiments, the formaldehyde fixation pathway comprises 1D. In certain embodiments, the formaldehyde fixation pathway comprises 1D, and the formate assimilation pathway comprises 1E. In certain embodiments, the formaldehyde fixation pathway comprises 1D, and the formate assimilation pathway comprises 1F, and 1G. In certain embodiments, the formaldehyde fixation pathway comprises 1D, and the formate assimilation pathway comprises 1H, 1I, 1J, and 1K. In certain embodiments, the formaldehyde fixation pathway comprises 1D, and the formate assimilation pathway comprises 1H, 1I, 1J, 1L, 1M, and 1N. In certain embodiments, the formaldehyde fixation pathway comprises 1D, and the formate assimilation pathway comprises 1E, 1H, 1I, 1J, 1L, 1M, and 1N. In certain embodiments, the formaldehyde fixation pathway comprises 1D, and the formate assimilation pathway comprises 1F, 1G, 1H, 1I, 1J, 1L, 1M, and 1N. In certain embodiments, the formaldehyde fixation pathway comprises 1D, and the formate assimilation pathway comprises 1K, 1H, 1I, 1J, 1L, 1M, and 1N. In certain embodiments, the formaldehyde fixation pathway comprises 1D, and the formate assimilation pathway comprises 1H, 1I, 1J, 1O, and 1P.

In certain embodiments, the formate assimilation pathway further comprises 1Q, 1R, or 1S or any combination thereof, wherein 1Q is a pyruvate formate lyase, wherein 1R is a pyruvate dehydrogenase, a pyruvate ferredoxin oxidoreductase, or a pyruvate:NADP+ oxidoreductase, wherein 1S is a formate dehydrogenase. Thus, in certain embodiments the formate assimilation pathway comprises 1Q. Thus, in certain embodiments the formate assimilation pathway comprises 1R. Thus, in certain embodiments the formate assimilation pathway comprises 1S.

In certain embodiments, formate assimilation pathway comprises 1Q, or 1R and 1S, and the formaldehyde fixation pathway comprises 1B and 1C. In certain embodiments, formate assimilation pathway comprises 1Q, or 1R and 1S, and the formaldehyde fixation pathway comprises 1D. In certain embodiments the formaldehyde fixation pathway comprises 1B and 1C, and the formate assimilation pathway comprises 1Q, and 1E. In certain embodiments, the formaldehyde fixation pathway comprises 1B and 1C, and the formate assimilation pathway comprises 1Q, 1F, and 1G. In certain embodiments, the formaldehyde fixation pathway comprises 1B and 1C, and the formate assimilation pathway comprises 1Q, 1H, 1I, 1J, and 1K. In certain embodiments, the formaldehyde fixation pathway comprises 1B and 1C, and the formate assimilation pathway comprises 1Q, 1H, 1I, 1J, 1L, 1M, and 1N. In certain embodiments, the formaldehyde fixation pathway comprises 1B and 1C, and the formate assimilation pathway comprises 1Q, 1E, 1H, 1I, 1J, 1L, 1M, and 1N. In certain embodiments, the formaldehyde fixation pathway comprises 1B and 1C, and the formate assimilation pathway comprises 1Q, 1F, 1G, 1H, 1I, 1J, 1L, 1M, and 1N. In certain embodiments, the formaldehyde fixation pathway comprises 1B and 1C, and the formate assimilation pathway comprises 1Q, 1K, 1H, 1I, 1J, 1L, 1M, and 1N. In certain embodiments, the formaldehyde fixation pathway comprises 1B and 1C, and the formate assimilation pathway comprises 1Q, 1H, 1I, 1J, 1O, and 1P. In certain embodiments the formaldehyde fixation pathway comprises 1D, and the formate assimilation pathway comprises 1Q, and 1E. In certain embodiments, the formaldehyde fixation pathway comprises 1D, and the formate assimilation pathway comprises 1Q, 1F, and 1G. In certain embodiments, the formaldehyde fixation pathway comprises 1D, and the formate assimilation pathway comprises 1Q, 1H, 1I, 1J, and 1K. In certain embodiments, the formaldehyde fixation pathway comprises 1D, and the formate assimilation pathway comprises 1Q, 1H, 1I, 1J, 1L, 1M, and 1N. In certain embodiments, the formaldehyde fixation pathway comprises 1D, and the formate assimilation pathway comprises 1Q, 1E, 1H, 1I, 1J, 1L, 1M, and 1N. In certain embodiments, the formaldehyde fixation pathway comprises 1D, and the formate assimilation pathway comprises 1Q, 1F, 1G, 1H, 1I, 1J, 1L, 1M, and 1N. In certain embodiments, the formaldehyde fixation pathway comprises 1D, and the formate assimilation pathway comprises 1Q, 1K, 1H, 1I, 1J, 1L, 1M, and 1N. In certain embodiments, the formaldehyde fixation pathway comprises 1D, and the formate assimilation pathway comprises 1Q, 1H, 1I, 1J, 1O, and 1P.

In certain embodiments, the formaldehyde fixation pathway or the formate assimilation pathway is a pathway depicted in FIG. 1 or 2.

In certain embodiments, provided herein is a non-naturally occurring microbial organism having a formaldehyde fixation pathway, a formate assimilation pathway and a methanol metabolic pathway. In some aspects, the organism comprises at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase, comprises at least one exogenous nucleic acid encoding a formate assimilation pathway enzyme expressed in a sufficient amount to produce formaldehyde, pyruvate, or acetyl-CoA, wherein said formate assimilation pathway comprises a pathway selected from: (3) 1E; (4) 1F, and 1G; (5) 1H, 1I, 1J, and 1K; (6) 1H, 1I, 1J, 1L, 1M, and 1N; (7) 1E, 1H, 1I, 1J, 1L, 1M, and 1N; (8) 1F, 1G, 1H, 1I, 1J, 1L, 1M, and 1N; (9) 1K, 1H, 1I, 1J, 1L, 1M, and 1N; and (10) 1H, 1I, 1J, 1O, and 1P5, and comprises at least one exogenous nucleic acid encoding a methanol metabolic pathway enzyme expressed in a sufficient amount to produce formaldehyde or produce or enhance the availability of reducing equivalents in the presence of methanol, wherein said methanol metabolic pathway comprises a pathway selected from: (1) 3J; (2) 3A and 3B; (3) 3A, 3B and 3C; (4) 3J, 3K and 3C; (5) 3J, 3M, and 3N; (6) 3J and 3L; (7) 3A, 3B, 3C, 3D, and 3E; (8) 3A, 3B, 3C, 3D, and 3F; (9) 3J, 3K, 3C, 3D, and 3E; (10) 3J, 3K, 3C, 3D, and 3F; (11) 3J, 3M, 3N, and 3O; (12) 3A, 3B, 3C, 3D, 3E, and 3G; (13) 3A, 3B, 3C, 3D, 3F, and 3G; (14) 3J, 3K, 3C, 3D, 3E, and 3G; (15) 3J, 3K, 3C, 3D, 3F, and 3G; (16) 3J, 3M, 3N, 3O, and 3G; (17) 3A, 3B, 3C, 3D, 3E, and 3I; (18) 3A, 3B, 3C, 3D, 3F, and 3I; (19) 3J, 3K, 3C, 3D, 3E, and 3I; (20) 3J, 3K, 3C, 3D, 3F, and 3I; and (21) 3J, 3M, 3N, 3O, and 3I, wherein 3A is a methanol methyltransferase, wherein 3B is a methylenetetrahydrofolate reductase, wherein 3C is a methylenetetrahydrofolate dehydrogenase, wherein 3D is a methenyltetrahydrofolate cyclohydrolase, wherein 3E is a formyltetrahydrofolate deformylase, wherein 3F is a formyltetrahydrofolate synthetase, wherein 3G is a formate hydrogen lyase, wherein 3H is a hydrogenase, wherein 3I is a formate dehydrogenase, wherein 3J is a methanol dehydrogenase, wherein 3K is a formaldehyde activating enzyme or spontaneous, wherein 3L is a formaldehyde dehydrogenase, wherein 3M is a S-(hydroxymethyl)glutathione synthase or spontaneous, wherein 3N is a glutathione-dependent formaldehyde dehydrogenase, wherein 3O is a S-formylglutathione hydrolase.

In certain embodiments, the methanol metabolic pathway comprises 3A. In certain embodiments, the methanol metabolic pathway comprises 3B. In certain embodiments, the methanol metabolic pathway comprises 3C. In certain embodiments, the methanol metabolic pathway comprises 3D. In certain embodiments, the methanol metabolic pathway comprises 3E. In certain embodiments, the methanol metabolic pathway comprises 3F. In certain embodiments, the methanol metabolic pathway comprises 3G. In certain embodiments, the methanol metabolic pathway comprises 3H. In certain embodiments, the methanol metabolic pathway comprises 3I. In certain embodiments, the methanol metabolic pathway comprises 3J. In certain embodiments, the methanol metabolic pathway comprises 3K. In certain embodiments, the methanol metabolic pathway comprises 3L. In certain embodiments, the methanol metabolic pathway comprises 3M. In certain embodiments, the methanol metabolic pathway comprises 3N. In certain embodiments, the methanol metabolic pathway comprises 3O.

In certain embodiments, the methanol metabolic pathway comprises 3J. In certain embodiments, the methanol metabolic pathway comprises 3A and 3B. In certain embodiments, the methanol metabolic pathway comprises 3A, 3B and 3C. In certain embodiments, the methanol metabolic pathway comprises 3J, 3K and 3C. In certain embodiments, the methanol metabolic pathway comprises 3J, 3M, and 3N. In certain embodiments, the methanol metabolic pathway comprises 3J and 3L. In certain embodiments, the methanol metabolic pathway comprises 3A, 3B, 3C, 3D, and 3E. In certain embodiments, the methanol metabolic pathway comprises 3A, 3B, 3C, 3D, and 3F. In certain embodiments, the methanol metabolic pathway comprises 3J, 3K, 3C, 3D, and 3E. In certain embodiments, the methanol metabolic pathway comprises 3J, 3K, 3C, 3D, and 3F. In certain embodiments, the methanol metabolic pathway comprises 3J, 3M, 3N, and 3O. In certain embodiments, the methanol metabolic pathway comprises 3A, 3B, 3C, 3D, 3E, and 3G. In certain embodiments, the methanol metabolic pathway comprises 3A, 3B, 3C, 3D, 3F, and 3G. In certain embodiments, the methanol metabolic pathway comprises 3J, 3K, 3C, 3D, 3E, and 3G. In certain embodiments, the methanol metabolic pathway comprises 3J, 3K, 3C, 3D, 3F, and 3G. In certain embodiments, the methanol metabolic pathway comprises 3J, 3M, 3N, 3O, and 3G. In certain embodiments, the methanol metabolic pathway comprises 3A, 3B, 3C, 3D, 3E, and 3I. In certain embodiments, the methanol metabolic pathway comprises 3A, 3B, 3C, 3D, 3F, and 3I. In certain embodiments, the methanol metabolic pathway comprises 3J, 3K, 3C, 3D, 3E, and 3I. In certain embodiments, the methanol metabolic pathway comprises 3J, 3K, 3C, 3D, 3F, and 3I. In certain embodiments, the methanol metabolic pathway comprises 3J, 3M, 3N, 3O, and 3I.

In certain embodiments, provided herein is a non-naturally occurring microbial organism having a formaldehyde fixation pathway, a formate assimilation pathway and a methanol oxidation pathway. In some aspects, the organism comprises at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase, comprises at least one exogenous nucleic acid encoding a formate assimilation pathway enzyme expressed in a sufficient amount to produce formaldehyde, pyruvate, or acetyl-CoA, wherein said formate assimilation pathway comprises a pathway selected from: (3) 1E; (4) 1F, and 1G; (5) 1H, 1I, 1J, and 1K; (6) 1H, 1I, 1J, 1L, 1M, and 1N; (7) 1E, 1H, 1I, 1J, 1L, 1M, and 1N; (8) 1F, 1G, 1H, 1I, 1J, 1L, 1M, and 1N; (9) 1K, 1H, 1I, 1J, 1L, 1M, and 1N; and (10) 1H, 1I, 1J, 1O, and 1P5, and comprises at least one exogenous nucleic acid encoding a methanol oxidation pathway enzyme expressed in a sufficient amount to produce formaldehyde in the presence of methanol, wherein said methanol oxidation pathway comprises 1A, wherein 1A a methanol dehydrogenase.

In certain embodiments, provided herein is a non-naturally occurring microbial organism having a formaldehyde fixation pathway and a methanol oxidation pathway. In some aspects, the organism comprises at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase, and comprises at least one exogenous nucleic acid encoding a methanol oxidation pathway enzyme expressed in a sufficient amount to produce formaldehyde in the presence of methanol, wherein said methanol oxidation pathway comprises 1A, wherein 1A a methanol dehydrogenase.

In certain embodiments, provided herein is a non-naturally occurring microbial organism having a formaldehyde fixation pathway, a formate assimilation pathway, a methanol metabolic pathway, and comprises 3H or 3P, wherein 3H is a hydrogenase, wherein 3P a carbon monoxide dehydrogenase. In some aspects, the organism comprises at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase, comprises at least one exogenous nucleic acid encoding a formate assimilation pathway enzyme expressed in a sufficient amount to produce formaldehyde, pyruvate, or acetyl-CoA, wherein said formate assimilation pathway comprises a pathway selected from: (3) 1E; (4) 1F, and 1G; (5) 1H, 1I, 1J, and 1K; (6) 1H, 1I, 1J, 1L, 1M, and 1N; (7) 1E, 1H, 1I, 1J, 1L, 1M, and 1N; (8) 1F, 1G, 1H, 1I, 1J, 1L, 1M, and 1N; (9) 1K, 1H, 1I, 1J, 1L, 1M, and 1N; and (10) 1H, 1I, 1J, 1O, and 1P5, and comprises at least one exogenous nucleic acid encoding a methanol metabolic pathway enzyme expressed in a sufficient amount to produce formaldehyde or produce or enhance the availability of reducing equivalents in the presence of methanol, wherein said methanol metabolic pathway comprises a pathway selected from: (1) 3J; (2) 3A and 3B; (3) 3A, 3B and 3C; (4) 3J, 3K and 3C; (5) 3J, 3M, and 3N; (6) 3J and 3L; (7) 3A, 3B, 3C, 3D, and 3E; (8) 3A, 3B, 3C, 3D, and 3F; (9) 3J, 3K, 3C, 3D, and 3E; (10) 3J, 3K, 3C, 3D, and 3F; (11) 3J, 3M, 3N, and 3O; (12) 3A, 3B, 3C, 3D, 3E, and 3G; (13) 3A, 3B, 3C, 3D, 3F, and 3G; (14) 3J, 3K, 3C, 3D, 3E, and 3G; (15) 3J, 3K, 3C, 3D, 3F, and 3G; (16) 3J, 3M, 3N, 3O, and 3G; (17) 3A, 3B, 3C, 3D, 3E, and 3I; (18) 3A, 3B, 3C, 3D, 3F, and 3I; (19) 3J, 3K, 3C, 3D, 3E, and 3I; (20) 3J, 3K, 3C, 3D, 3F, and 3I; and (21) 3J, 3M, 3N, 3O, and 3I, wherein 3A is a methanol methyltransferase, wherein 3B is a methylenetetrahydrofolate reductase, wherein 3C is a methylenetetrahydrofolate dehydrogenase, wherein 3D is a methenyltetrahydrofolate cyclohydrolase, wherein 3E is a formyltetrahydrofolate deformylase, wherein 3F is a formyltetrahydrofolate synthetase, wherein 3G is a formate hydrogen lyase, wherein 3H is a hydrogenase, wherein 3I is a formate dehydrogenase, wherein 3J is a methanol dehydrogenase, wherein 3K is a formaldehyde activating enzyme or spontaneous, wherein 3L is a formaldehyde dehydrogenase, wherein 3M is a S-(hydroxymethyl)glutathione synthase or spontaneous, wherein 3N is a glutathione-dependent formaldehyde dehydrogenase, wherein 3O is a S-formylglutathione hydrolase, wherein said microbial organism further comprises 3H or 3P, wherein 3H is a hydrogenase, wherein 3P a carbon monoxide dehydrogenase.

In certain embodiments, provided herein is a non-naturally occurring microbial organism having a formaldehyde fixation pathway, a formate assimilation pathway, a methanol oxidation pathway, and comprises 3H or 3P, wherein 3H is a hydrogenase, wherein 3P a carbon monoxide dehydrogenase. In some aspects, the organism comprises at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase, comprises at least one exogenous nucleic acid encoding a formate assimilation pathway enzyme expressed in a sufficient amount to produce formaldehyde, pyruvate, or acetyl-CoA, wherein said formate assimilation pathway comprises a pathway selected from: (3) 1E; (4) 1F, and 1G; (5) 1H, 1I, 1J, and 1K; (6) 1H, 1I, 1J, 1L, 1M, and 1N; (7) 1E, 1H, 1I, 1J, 1L, 1M, and 1N; (8) 1F, 1G, 1H, 1I, 1J, 1L, 1M, and 1N; (9) 1K, 1H, 1I, 1J, 1L, 1M, and 1N; and (10) 1H, 1I, 1J, 1O, and 1P5, and comprises at least one exogenous nucleic acid encoding a methanol oxidation pathway enzyme expressed in a sufficient amount to produce formaldehyde in the presence of methanol, wherein said methanol oxidation pathway comprises 1A, wherein 1A a methanol dehydrogenase, wherein said microbial organism further comprises 3H or 3P, wherein 3H is a hydrogenase, wherein 3P a carbon monoxide dehydrogenase.

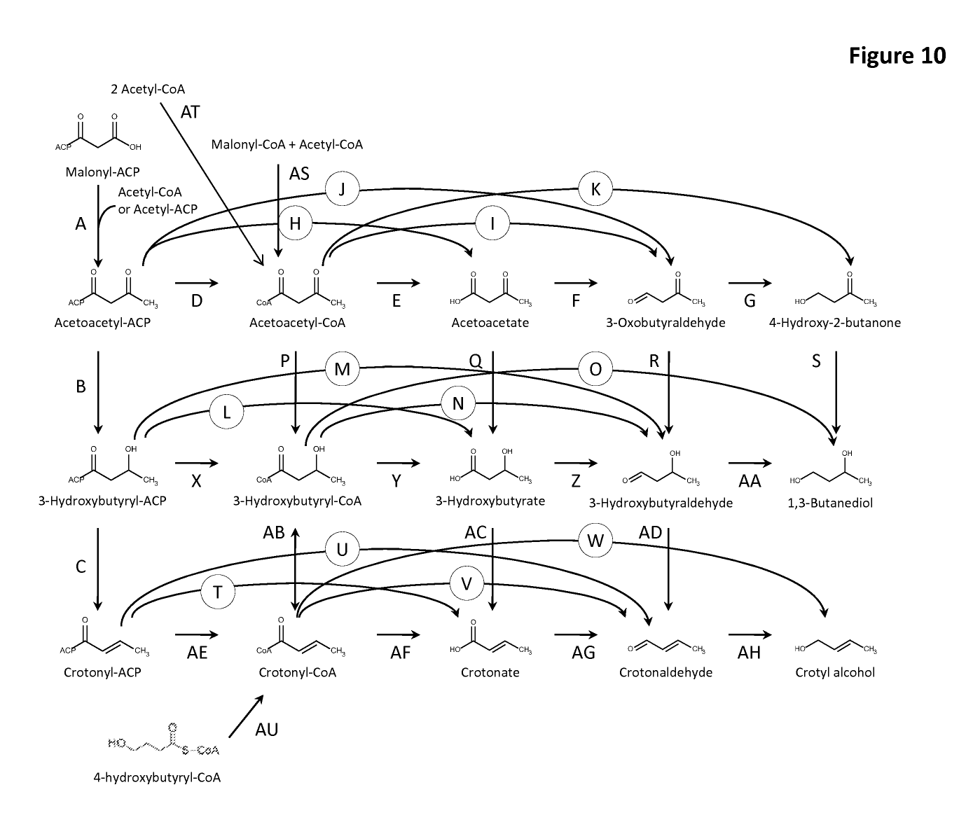

In some embodiments, the invention provides a non-naturally occurring microbial organism having a butadiene pathway including at least one exogenous nucleic acid encoding a butadiene pathway enzyme expressed in a sufficient amount to produce butadiene, wherein the butadiene pathway includes a pathway shown in FIGS. 10 and 13-18 selected from: (1) 10A, 10D, 10E, 10F, 10G, 10S, 15A, 15B, 15C, and 15G; (2) 10A, 10D, 10I, 10G, 10S, 15A, 15B, 15C, and 15G; (3) 10A, 10D, 10K, 10S, 15A, 15B, 15C, and 15G; (4) 10A, 10H, 10F, 10G, 10S, 15A, 15B, 15C, and 15G; (5) 10A, 10J, 10G, 10S, 15A, 15B, 15C, and 15G; (6) 10A, 10J, 10R, 10AA, 15A, 15B, 15C, and 15G; (7) 10A, 10H, 10F, 10R, 10AA, 15A, 15B, 15C, and 15G; (8) 10A, 10H, 10Q, 10Z, 10AA, 15A, 15B, 15C, and 15G; (9) 10A, 10D, 10I, 10R, 10AA, 15A, 15B, 15C, and 15G; (10) 10A, 10D, 10E, 10F, 10R, 10AA, 15A, 15B, 15C, and 15G; (11) 10A, 10D, 10E, 10Q, 10Z, 10AA, 15A, 15B, 15C, and 15G; (12) 10A, 10D, 10P, 10N, 10AA, 15A, 15B, 15C, and 15G; (13) 10A, 10D, 10P, 10Y, 10Z, 10AA, 15A, 15B, 15C, and 15G; (14) 10A, 10B, 10M, 10AA, 15A, 15B, 15C, and 15G; (15) 10A, 10B, 10L, 10Z, 10AA, 15A, 15B, 15C, and 15G; (16) 10A, 10B, 10X, 10N, 10AA, 15A, 15B, 15C, and 15G; (17) 10A, 10B, 10X, 10Y, 10Z, 10AA, 15A, 15B, 15C, and 15G; (18) 10A, 10D, 10P, 10O, 15A, 15B, 15C, and 15G; (19) 10A, 10B, 10X, 10O, 15A, 15B, 15C, and 15G; (20) 10A, 10D, 10E, 10F, 10R, 10AA, 15A, 15B, 15C, and 15G; (21) 10A, 10D, 10E, 10F, 10G, 10S, 15A, 15B, 15C, and 15G; (22) 10A, 10B, 10C, 10AE, 10AB, 10Y, 10Z, 10AA, 15A, 15B, 15C, and 15G; (23) 10A, 10B, 10C, 10AE, 10AB, 10N, 10AA, 15A, 15B, 15C, and 15G; (24) 10A, 10B, 10C, 10AE, 10AB, 10O, 15A, 15B, 15C, and 15G; (25) 10AU, 10AB, 10Y, 10Z, 10AA, 15A, 15B, 15C, and 15G; (26) 10AU, 10AB, 10N, 10AA, 15A, 15B, 15C, and 15G; (27) 10AU, 10AB, 10O, 15A, 15B, 15C, and 15G; (28) 1T, 10AS, 10E, 10F, 10G, 10S, 15A, 15B, 15C, and 15G; (29) 1T, 10AS, 10I, 10G, 10S, 15A, 15B, 15C, and 15G; (30) 1T, 10AS, 10K, 10S, 15A, 15B, 15C, and 15G; (31) 1T, 10AS, 10I, 10R, 10AA, 15A, 15B, 15C, and 15G; (32) 1T, 10AS, 10E, 10F, 10R, 10AA, 15A, 15B, 15C, and 15G; (33) 1T, 10AS, 10E, 10Q, 10Z, 10AA, 15A, 15B, 15C, and 15G; (34) 1T, 10AS, 10P, 10N, 10AA, 15A, 15B, 15C, and 15G; (35) 1T, 10AS, 10P, 10Y, 10Z, 10AA, 15A, 15B, 15C, and 15G; (36) 1T, 10AS, 10P, 10O, 15A, 15B, 15C, and 15G; (37) 1T, 10AS, 10E, 10F, 10R, 10AA, 15A, 15B, 15C, and 15G; (38) 10AT, 10E, 10F, 10G, 10S, 15A, 15B, 15C, and 15G; (39) 10AT, 10I, 10G, 10S, 15A, 15B, 15C, and 15G; (40) 10AT, 10K, 10S, 15A, 15B, 15C, and 15G; (41) 10AT, 10I, 10R, 10AA, 15A, 15B, 15C, and 15G; (42) 10AT, 10E, 10F, 10R, 10AA, 15A, 15B, 15C, and 15G; (43) 10AT, 10E, 10Q, 10Z, 10AA, 15A, 15B, 15C, and 15G; (44) 10AT, 10P, 10N, 10AA, 15A, 15B, 15C, and 15G; (45) 10AT, 10P, 10Y, 10Z, 10AA, 15A, 15B, 15C, and 15G; (46) 10AT, 10P, 10O, 15A, 15B, 15C, and 15G; (47) 10AT, 10E, 10F, 10R, 10AA, 15A, 15B, 15C, and 15G; (48) 10A, 10D, 10E, 10F, 10G, 10S, 15D, and 15G; (49) 10A, 10D, 10I, 10G, 10S, 15D, and 15G; (50) 10A, 10D, 10K, 10S, 15D, and 15G; (51) 10A, 10H, 10F, 10G, 10S, 15D, and 15G; (52) 10A, 10J, 10G, 10S, 15D, and 15G; (53) 10A, 10J, 10R, 10AA, 15D, and 15G; (54) 10A, 10H, 10F, 10R, 10AA, 15D, and 15G; (55) 10A, 10H, 10Q, 10Z, 10AA, 15D, and 15G; (56) 10A, 10D, 10I, 10R, 10AA, 15D, and 15G; (57) 10A, 10D, 10E, 10F, 10R, 10AA, 15D, and 15G; (58) 10A, 10D, 10E, 10Q, 10Z, 10AA, 15D, and 15G; (59) 10A, 10D, 10P, 10N, 10AA, 15D, and 15G; (60) 10A, 10D, 10P, 10Y, 10Z, 10AA, 15D, and 15G; (61) 10A, 10B, 10M, 10AA, 15D, and 15G; (62) 10A, 10B, 10L, 10Z, 10AA, 15D, and 15G; (63) 10A, 10B, 10X, 10N, 10AA, 15D, and 15G; (64) 10A, 10B, 10X, 10Y, 10Z, 10AA, 15D, and 15G; (65) 10A, 10D, 10P, 10O, 15D, and 15G; (66) 10A, 10B, 10X, 10O, 15D, and 15G; (67) 10A, 10D, 10E, 10F, 10R, 10AA, 15D, and 15G; (68) 10A, 10D, 10E, 10F, 10G, 10S, 15D, and 15G; (69) 10A, 10B, 10C, 10AE, 10AB, 10Y, 10Z, 10AA, 15D, and 15G; (70) 10A, 10B, 10C, 10AE, 10AB, 10N, 10AA, 15D, and 15G; (71) 10A, 10B, 10C, 10AE, 10AB, 10O, 15D, and 15G; (72) 10AU, 10AB, 10Y, 10Z, 10AA, 15D, and 15G; (73) 10AU, 10AB, 10N, 10AA, 15D, and 15G; (74) 10AU, 10AB, 10O, 15D, and 15G; (75) 1T, 10AS, 10E, 10F, 10G, 10S, 15D, and 15G; (76) 1T, 10AS, 10I, 10G, 10S, 15D, and 15G; (77) 1T, 10AS, 10K, 10S, 15D, and 15G; (78) 1T, 10AS, 10I, 10R, 10AA, 15D, and 15G; (79) 1T, 10AS, 10E, 10F, 10R, 10AA, 15D, and 15G; (80) 1T, 10AS, 10E, 10Q, 10Z, 10AA, 15D, and 15G; (81) 1T, 10AS, 10P, 10N, 10AA, 15D, and 15G; (82) 1T, 10AS, 10P, 10Y, 10Z, 10AA, 15D, and 15G; (83) 1T, 10AS, 10P, 10O, 15D, and 15G; (84) 1T, 10AS, 10E, 10F, 10R, 10AA, 15D, and 15G; (85) 10AT, 10E, 10F, 10G, 10S, 15D, and 15G; (86) 10AT, 10I, 10G, 10S, 15D, and 15G; (87) 10AT, 10K, 10S, 15D, and 15G; (88) 10AT, 10I, 10R, 10AA, 15D, and 15G; (89) 10AT, 10E, 10F, 10R, 10AA, 15D, and 15G; (90) 10AT, 10E, 10Q, 10Z, 10AA, 15D, and 15G; (91) 10AT, 10P, 10N, 10AA, 15D, and 15G; (92) 10AT, 10P, 10Y, 10Z, 10AA, 15D, and 15G; (93) 10AT, 10P, 10O, 15D, and 15G; (94) 10AT, 10E, 10F, 10R, 10AA, 15D, and 15G; (95) 10A, 10D, 10E, 10F, 10G, 10S, 15E, 15C, and 15G; (96) 10A, 10D, 10I, 10G, 10S, 15E, 15C, and 15G; (97) 10A, 10D, 10K, 10S, 15E, 15C, and 15G; (98) 10A, 10H, 10F, 10G, 10S, 15E, 15C, and 15G; (99) 10A, 10J, 10G, 10S, 15E, 15C, and 15G; (100) 10A, 10J, 10R, 10AA, 15E, 15C, and 15G; (101) 10A, 10H, 10F, 10R, 10AA, 15E, 15C, and 15G; (102) 10A, 10H, 10Q, 10Z, 10AA, 15E, 15C, and 15G; (103) 10A, 10D, 10I, 10R, 10AA, 15E, 15C, and 15G; (104) 10A, 10D, 10E, 10F, 10R, 10AA, 15E, 15C, and 15G; (105) 10A, 10D, 10E, 10Q, 10Z, 10AA, 15E, 15C, and 15G; (106) 10A, 10D, 10P, 10N, 10AA, 15E, 15C, and 15G; (107) 10A, 10D, 10P, 10Y, 10Z, 10AA, 15E, 15C, and 15G; (108) 10A, 10B, 10M, 10AA, 15E, 15C, and 15G; (109) 10A, 10B, 10L, 10Z, 10AA, 15E, 15C, and 15G; (110) 10A, 10B, 10X, 10N, 10AA, 15E, 15C, and 15G; (111) 10A, 10B, 10X, 10Y, 10Z, 10AA, 15E, 15C, and 15G; (112) 10A, 10D, 10P, 10O, 15E, 15C, and 15G; (113) 10A, 10B, 10X, 10O, 15E, 15C, and 15G; (114) 10A, 10D, 10E, 10F, 10R, 10AA, 15E, 15C, and 15G; (115) 10A, 10D, 10E, 10F, 10G, 10S, 15E, 15C, and 15G; (116) 10A, 10B, 10C, 10AE, 10AB, 10Y, 10Z, 10AA, 15E, 15C, and 15G; (117) 10A, 10B, 10C, 10AE, 10AB, 10N, 10AA, 15E, 15C, and 15G; (118) 10A, 10B, 10C, 10AE, 10AB, 10O, 15E, 15C, and 15G; (119) 10AU, 10AB, 10Y, 10Z, 10AA, 15E, 15C, and 15G; (120) 10AU, 10AB, 10N, 10AA, 15E, 15C, and 15G; (121) 10AU, 10AB, 10O, 15E, 15C, and 15G; (122) 1T, 10AS, 10E, 10F, 10G, 10S, 15E, 15C, and 15G; (123) 1T, 10AS, 10I, 10G, 10S, 15E, 15C, and 15G; (124) 1T, 10AS, 10K, 10S, 15E, 15C, and 15G; (125) 1T, 10AS, 10I, 10R, 10AA, 15E, 15C, and 15G; (126) 1T, 10AS, 10E, 10F, 10R, 10AA, 15E, 15C, and 15G; (127) 1T, 10AS, 10E, 10Q, 10Z, 10AA, 15E, 15C, and 15G; (128) 1T, 10AS, 10P, 10N, 10AA, 15E, 15C, and 15G; (129) 1T, 10AS, 10P, 10Y, 10Z, 10AA, 15E, 15C, and 15G; (130) 1T, 10AS, 10P, 10O, 15E, 15C, and 15G; (131) 1T, 10AS, 10E, 10F, 10R, 10AA, 15E, 15C, and 15G; (132) 10AT, 10E, 10F, 10G, 10S, 15E, 15C, and 15G; (133) 10AT, 10I, 10G, 10S, 15E, 15C, and 15G; (134) 10AT, 10K, 10S, 15E, 15C, and 15G; (135) 10AT, 10I, 10R, 10AA, 15E, 15C, and 15G; (136) 10AT, 10E, 10F, 10R, 10AA, 15E, 15C, and 15G; (137) 10AT, 10E, 10Q, 10Z, 10AA, 15E, 15C, and 15G; (138) 10AT, 10P, 10N, 10AA, 15E, 15C, and 15G; (139) 10AT, 10P, 10Y, 10Z, 10AA, 15E, 15C, and 15G; (140) 10AT, 10P, 10O, 15E, 15C, and 15G; (141) 10AT, 10E, 10F, 10R, 10AA, 15E, 15C, and 15G; (142) 10A, 10D, 10E, 10F, 10G, 10S, 15A, 15F, and 15G; (143) 10A, 10D, 10I, 10G, 10S, 15A, 15F, and 15G; (144) 10A, 10D, 10K, 10S, 15A, 15F, and 15G; (145) 10A, 10H, 10F, 10G, 10S, 15A, 15F, and 15G; (146) 10A, 10J, 10G, 10S, 15A, 15F, and 15G; (147) 10A, 10J, 10R, 10AA, 15A, 15F, and 15G; (148) 10A, 10H, 10F, 10R, 10AA, 15A, 15F, and 15G; (149) 10A, 10H, 10Q, 10Z, 10AA, 15A, 15F, and 15G; (150) 10A, 10D, 10I, 10R, 10AA, 15A, 15F, and 15G; (151) 10A, 10D, 10E, 10F, 10R, 10AA, 15A, 15F, and 15G; (152) 10A, 10D, 10E, 10Q, 10Z, 10AA, 15A, 15F, and 15G; (153) 10A, 10D, 10P, 10N, 10AA, 15A, 15F, and 15G; (154) 10A, 10D, 10P, 10Y, 10Z, 10AA, 15A, 15F, and 15G; (155) 10A, 10B, 10M, 10AA, 15A, 15F, and 15G; (156) 10A, 10B, 10L, 10Z, 10AA, 15A, 15F, and 15G; (157) 10A, 10B, 10X, 10N, 10AA, 15A, 15F, and 15G; (158) 10A, 10B, 10X, 10Y, 10Z, 10AA, 15A, 15F, and 15G; (159) 10A, 10D, 10P, 10O, 15A, 15F, and 15G; (160) 10A, 10B, 10X, 10O, 15A, 15F, and 15G; (161) 10A, 10D, 10E, 10F, 10R, 10AA, 15A, 15F, and 15G; (162) 10A, 10D, 10E, 10F, 10G, 10S, 15A, 15F, and 15G; (163) 10A, 10B, 10C, 10AE, 10AB, 10Y, 10Z, 10AA, 15A, 15F, and 15G; (164) 10A, 10B, 10C, 10AE, 10AB, 10N, 10AA, 15A, 15F, and 15G; (165) 10A, 10B, 10C, 10AE, 10AB, 10O, 15A, 15F, and 15G; (166) 10AU, 10AB, 10Y, 10Z, 10AA, 15A, 15F, and 15G; (167) 10AU, 10AB, 10N, 10AA, 15A, 15F, and 15G; (168) 10AU, 10AB, 10O, 15A, 15F, and 15G; (169) 1T, 10AS, 10E, 10F, 10G, 10S, 15A, 15F, and 15G; (170) 1T, 10AS, 10I, 10G, 10S, 15A, 15F, and 15G; (171) 1T, 10AS, 10K, 10S, 15A, 15F, and 15G; (172) 1T, 10AS, 10I, 10R, 10AA, 15A, 15F, and 15G; (173) 1T, 10AS, 10E, 10F, 10R, 10AA, 15A, 15F, and 15G; (174) 1T, 10AS, 10E, 10Q, 10Z, 10AA, 15A, 15F, and 15G; (175) 1T, 10AS, 10P, 10N, 10AA, 15A, 15F, and 15G; (176) 1T, 10AS, 10P, 10Y, 10Z, 10AA, 15A, 15F, and 15G; (177) 1T, 10AS, 10P, 10O, 15A, 15F, and 15G; (178) 1T, 10AS, 10E, 10F, 10R, 10AA, 15A, 15F, and 15G; (179) 10AT, 10E, 10F, 10G, 10S, 15A, 15F, and 15G; (180) 10AT, 10I, 10G, 10S, 15A, 15F, and 15G; (181) 10AT, 10K, 10S, 15A, 15F, and 15G; (182) 10AT, 10I, 10R, 10AA, 15A, 15F, and 15G; (183) 10AT, 10E, 10F, 10R, 10AA, 15A, 15F, and 15G; (184) 10AT, 10E, 10Q, 10Z, 10AA, 15A, 15F, and 15G; (185) 10AT, 10P, 10N, 10AA, 15A, 15F, and 15G; (186) 10AT, 10P, 10Y, 10Z, 10AA, 15A, 15F, and 15G; (187) 10AT, 10P, 10O, 15A, 15F, and 15G; (188) 10AT, 10E, 10F, 10R, 10AA, 15A, 15F, and 15G; (189) 14A, 14B, 14C, 14D, 14E, 13A, and 13B; (190) 15A, 15B, 15C, and 15G; (191) 15D, and 15G; (192) 15E, 15C, and 15G; (193) 15A, 15F, and 15G; (194) 16A, 16B, 16C, 16D, and 16E; (195) 17A, 17B, 17C, 17D, and 17G; (196) 17A, 17E, 17F, 17D, and 17G; (197) 17A, 17B, 17C, 17H, 17I, 17J, and 17G; (198) 18A, 18B, 18C, 18D, 18E, and 18F; (199) 13A, and 13B; and (200) 17A, 17E, 17F, 17H, 17I, 17J, and 17G, wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AE is a crotonyl-CoA:ACP transferase, wherein 10AS is an acetoacetyl-CoA synthase, wherein 10AT is an acetyl-CoA:acetyl-CoA acyltransferase, wherein 10AU is a 4-hydroxybutyryl-CoA dehydratase, wherein 13A is a 2-butanol desaturase, wherein 13B is a 3-buten-2-ol dehydratase, wherein 14A is an acetolactate synthase, wherein 14B is an acetolactate decarboxylase, wherein 14C is a butanediol dehydrogenase, wherein 14D is a butanediol dehydratase, wherein 14E is a butanol dehydrogenase, wherein 15A is a 1,3-butanediol kinase, wherein 15B is a 3-hydroxybutyrylphosphate kinase, 15C is a 3-hydroxybutyryldiphosphate lyase, wherein 15D is a 1,3-butanediol diphosphokinase, wherein 15E is a 1,3-butanediol dehydratase, wherein 15F is a 3-hydroxybutyrylphosphate lyase, wherein 15G is a 3-buten-2-ol dehydratase, wherein 16A is a 3-oxopent-4-enoyl-CoA thiolase, wherein 16B is a 3-oxopent-4-enoyl-CoA hydrolase, synthetase or transferase, wherein 16C is a 3-oxopent-4-enoate decarboxylase or spontaneous, wherein 16D is a 3-buten-2-one reductase, wherein 16E is a 3-buten-2-ol dehydratase, wherein 17A is a 3-oxo-4-hydroxypentanoyl-CoA thiolase, wherein 17B is a 3-oxo-4-hydroxypentanoyl-CoA transferase, synthetase or hydrolase, wherein 17C is a 3-oxo-4-hydroxypentanoate reductase, wherein 17D is a 3,4-dihydroxypentanoate decarboxylase, wherein 17E is a 3-oxo-4-hydroxypentanoyl-CoA reductase, wherein 17F is a 3,4-dihydroxypentanoyl-CoA transferase, synthetase or hydrolase, wherein 17G is a 3-buten-2-ol dehydratase, wherein 17H is a 3,4-dihydroxypentanoate dehydratase, wherein 17I is a 4-oxopentanoate reductase, wherein 17J is a 4-hyd4-oxoperoxypentanoate decarboxylase, wherein 18A is a 3-oxoadipyl-CoA thiolase, wherein 18B is a 3-oxoadipyl-CoA transferase, synthetase or hydrolase, wherein 18C is a 3-oxoadipate decarboxylase or spontaneous, wherein 18D is a 4-oxopentanoate reductase, wherein 18E is a 4-hydroxypentanoate decarboxylase, wherein 18F is a 3-buten-2-ol dehydratase.

In one aspect, the non-naturally occurring microbial organism a butadiene pathway described above further comprises a formaldehyde fixation pathway comprising at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase.

In one aspect, the non-naturally occurring microbial organism having a butadiene pathway described above further comprises a methanol metabolic pathway. In certain embodiments, the organism comprises at least one exogenous nucleic acid encoding a methanol metabolic pathway enzyme expressed in a sufficient amount to produce formaldehyde or produce or enhance the availability of reducing equivalents in the presence of methanol, wherein said methanol metabolic pathway comprises a pathway selected from: (1) 3J; (2) 3A and 3B; (3) 3A, 3B and 3C; (4) 3J, 3K and 3C; (5) 3J, 3M, and 3N; (6) 3J and 3L; (7) 3A, 3B, 3C, 3D, and 3E; (8) 3A, 3B, 3C, 3D, and 3F; (9) 3J, 3K, 3C, 3D, and 3E; (10) 3J, 3K, 3C, 3D, and 3F; (11) 3J, 3M, 3N, and 3O; (12) 3A, 3B, 3C, 3D, 3E, and 3G; (13) 3A, 3B, 3C, 3D, 3F, and 3G; (14) 3J, 3K, 3C, 3D, 3E, and 3G; (15) 3J, 3K, 3C, 3D, 3F, and 3G; (16) 3J, 3M, 3N, 3O, and 3G; (17) 3A, 3B, 3C, 3D, 3E, and 3I; (18) 3A, 3B, 3C, 3D, 3F, and 3I; (19) 3J, 3K, 3C, 3D, 3E, and 3I; (20) 3J, 3K, 3C, 3D, 3F, and 31; and (21) 3J, 3M, 3N, 3O, and 3I, wherein 3A is a methanol methyltransferase, wherein 3B is a methylenetetrahydrofolate reductase, wherein 3C is a methylenetetrahydrofolate dehydrogenase, wherein 3D is a methenyltetrahydrofolate cyclohydrolase, wherein 3E is a formyltetrahydrofolate deformylase, wherein 3F is a formyltetrahydrofolate synthetase, wherein 3G is a formate hydrogen lyase, wherein 3H is a hydrogenase, wherein 3I is a formate dehydrogenase, wherein 3J is a methanol dehydrogenase, wherein 3K is a formaldehyde activating enzyme or spontaneous, wherein 3L is a formaldehyde dehydrogenase, wherein 3M is a S-(hydroxymethyl)glutathione synthase or spontaneous, wherein 3N is a glutathione-dependent formaldehyde dehydrogenase, wherein 3O is a S-formylglutathione hydrolase,

In one aspect, the non-naturally occurring microbial organism having a butadiene pathway described above further comprises a methanol oxidation pathway. In certain embodiments, the organism comprises at least one exogenous nucleic acid encoding a methanol oxidation pathway enzyme expressed in a sufficient amount to produce formaldehyde in the presence of methanol, wherein said methanol oxidation pathway comprises 1A, wherein 1A a methanol dehydrogenase.

In one aspect, the non-naturally occurring microbial organism having a butadiene pathway described above further comprises 3H or 3P, wherein 3H is a hydrogenase, wherein 3P a carbon monoxide dehydrogenase. In certain embodiments, the organism comprises an exogenous nucleic acid encoding said hydrogenase or said carbon monoxide dehydrogenase.

In certain embodiments, provided herein is a non-naturally occurring microbial organism having a formaldehyde fixation pathway, a formate assimilation pathway, a methanol metabolic pathway, a methanol oxidation pathway, a hydrogenase, a carbon monoxide dehydrogenase or any combination described above, wherein the organism further comprises a butadiene pathway. In certain embodiments, the microbial organism comprises at least one exogenous nucleic acid encoding a butadiene pathway enzyme expressed in a sufficient amount to produce butadiene, wherein said butadiene pathway as shown in FIGS. 1, 2, and 10-18 comprises a pathway selected from: (1) 10A, 10J, 10R, 10AD, 10AH, 11A, 11B, and 11C; (2) 10A, 10H, 10F, 10R, 10AD, 10AH, 11A, 11B, and 11C; (3) 10A, 10H, 10Q, 10Z, 10AD, 10AH, 11A, 11B, and 11C; (4) 10A, 10H, 10Q, 10AC, 10AG, 10AH, 11A, 11B, and 11C; (5) 10A, 10D, 10I, 10R, 10AD, 10AH, 11A, 11B, and 11C; (6) 10A, 10D, 10E, 10F, 10R, 10AD, 10AH, 11A, 11B, and 11C; (7) 10A, 10D, 10E, 10Q, 10Z, 10AD, 10AH, 11A, 11B, and 11C; (8) 10A, 10D, 10E, 10Q, 10AC, 10AG, 10AH, 11A, 11B, and 11C; (9) 10A, 10D, 10P, 10N, 10AD, 10AH, 11A, 11B, and 11C; (10) 10A, 10D, 10P, 10Y, 10Z, 10AD, 10AH, 11A, 11B, and 11C; (11) 10A, 10D, 10P, 10Y, 10AC, 10AG, 10AH, 11A, 11B, and 11C; (12) 10A, 10D, 10P, 10AB, 10V, 10AH, 11A, 11B, and 11C; (13) 10A, 10D, 10P, 10AB, 10AF, 10AG, 10AH, 11A, 11B, and 11C; (14) 10A, 10B, 10M, 10AD, 10AH, 11A, 11B, and 11C; (15) 10A, 10B, 10L, 10Z, 10AD, 10AH, 11A, 11B, and 11C; (16) 10A, 10B, 10L, 10AC, 10AG, 10AH, 11A, 11B, and 11C; (17) 10A, 10B, 10X, 10Y, 10Z, 10AD, 10AH, 11A, 11B, and 11C; (18) 10A, 10B, 10X, 10Y, 10AC, 10AG, 10AH, 11A, 11B, and 11C; (19) 10A, 10B, 10X, 10AB, 10V, 10AH, 11A, 11B, and 11C; (20) 10A, 10B, 10X, 10AB, 10AF, 10AG, 10AH, 11A, 11B, and 11C; (21) 10A, 10B, 10C, 10U, 10AH, 11A, 11B, and 11C; (22) 10A, 10B, 10C, 10T, 10AG, 10AH, 11A, 11B, and 11C; (23) 10A, 10B, 10C, 10AE, 10AF, 10AG, 10AH, 11A, 11B, and 11C; (24) 10A, 10D, 10P, 10AB, 10W, 11A, 11B, and 11C; (25) 10A, 10B, 10X, 10AB, 10W, 11A, 11B, and 11C; (26) 10A, 10B, 10C, 10AE, 10W, 11A, 11B, and 11C; (27) 10A, 10B, 10C, 10AE, 10V, 10AH, 11A, 11B, and 11C (28) 10A, 10J, 10R, 10AD, 10AH, 11D, and 11C; (29) 10A, 10H, 10F, 10R, 10AD, 10AH, 11D, and 11C; (30) 10A, 10H, 10Q, 10Z, 10AD, 10AH, 11D, and 11C; (31) 10A, 10H, 10Q, 10AC, 10AG, 10AH, 11D, and 11C; (32) 10A, 10D, 10T, 10R, 10AD, 10AH, 11D, and 11C; (33) 10A, 10D, 10E, 10F, 10R, 10AD, 10AH, 11D, and 11C; (34) 10A, 10D, 10E, 10Q, 10Z, 10AD, 10AH, 11D, and 11C; (35) 10A, 10D, 10E, 10Q, 10AC, 10AG, 10AH, 11D, and 11C; (36) 10A, 10D, 10P, 10N, 10AD, 10AH, 11D, and 11C; (37) 10A, 10D, 10P, 10Y, 10Z, 10AD, 10AH, 11D, and 11C; (38) 10A, 10D, 10P, 10Y, 10AC, 10AG, 10AH, 11D, and 11C; (39) 10A, 10D, 10P, 10AB, 10V, 10AH, 11D, and 11C; (40) 10A, 10D, 10P, 10AB, 10AF, 10AG, 10AH, 11D, and 11C; (41) 10A, 10B, 10M, 10AD, 10AH, 11D, and 11C; (42) 10A, 10B, 10L, 10Z, 10AD, 10AH, 11D, and 11C; (43) 10A, 10B, 10L, 10AC, 10AG, 10AH, 11D, and 11C; (44) 10A, 10B, 10X, 10Y, 10Z, 10AD, 10AH, 11D, and 11C; (45) 10A, 10B, 10X, 10Y, 10AC, 10AG, 10AH, 11D, and 11C; (46) 10A, 10B, 10X, 10AB, 10V, 10AH, 11D, and 11C; (47) 10A, 10B, 10X, 10AB, 10AF, 10AG, 10AH, 11D, and 11C; (48) 10A, 10B, 10C, 10U, 10AH, 11D, and 11C; (49) 10A, 10B, 10C, 10T, 10AG, 10AH, 11D, and 11C; (50) 10A, 10B, 10C, 10AE, 10AF, 10AG, 10AH, 11D, and 11C; (51) 10A, 10D, 10P, 10AB, 10W, 11D, and 11C; (52) 10A, 10B, 10X, 10AB, 10W, 11D, and 11C; (53) 10A, 10B, 10C, 10AE, 10W, 11D, and 11C; (54) 10A, 10B, 10C, 10AE, by, 10AH, 11D, and 11C; (55) 10T, 10R, 10AD, 10AH, 11A, 11B, and 11C; (56) 10E, 10F, 10R, 10AD, 10AH, 11A, 11B, and 11C; (57) 10E, 10Q, 10Z, 10AD, 10AH, 11A, 11B, and 11C; (58) 10E, 10Q, 10AC, 10AG, 10AH, 11A, 11B, and 11C; (59) 10P, 10N, 10AD, 10AH, 11A, 11B, and 11C; (60) 10P, 10Y, 10Z, 10AD, 10AH, 11A, 11B, and 11C; (61) 10P, 10Y, 10AC, 10AG, 10AH, 11A, 11B, and 11C; (62) 10P, 10AB, 10V, 10AH, 11A, 11B, and 11C; (63) 10P, 10AB, 10AF, 10AG, 10AH, 11A, 11B, and 11C; (64) 10P, 10AB, 10W, 11A, 11B, and 11C; (65) 10T, 10R, 10AD, 10AH, 11D, and 11C; (66) 10E, 10F, 10R, 10AD, 10AH, 11D, and 11C; (67) 10E, 10Q, 10Z, 10AD, 10AH, 11D, and 11C; (68) 10E, 10Q, 10AC, 10AG, 10AH, 11D, and 11C; (69) 10P, 10N, 10AD, 10AH, 11D, and 11C; (70) 10P, 10Y, 10Z, 10AD, 10AH, 11D, and 11C; (71) 10P, 10Y, 10AC, 10AG, 10AH, 11D, and 11C; (72) 10P, 10AB, 10V, 10AH, 11D, and 11C; (73) 10P, 10AB, 10AF, 10AG, 10AH, 11D, and 11C; (74) 10P, 10AB, 10W, 11D, and 11C; (75) 1T, 10AS, 10T, 10R, 10AD, 10AH, 11A, 11B, and 11C; (76) 1T, 10AS, 10E, 10F, 10R, 10AD, 10AH, 11A, 11B, and 11C; (77) 1T, 10AS, 10E, 10Q, 10Z, 10AD, 10AH, 11A, 11B, and 11C; (78) 1T, 10AS, 10E, 10Q, 10AC, 10AG, 10AH, 11A, 11B, and 11C; (79) 1T, 10AS, 10P, 10N, 10AD, 10AH, 11A, 11B, and 11C; (80) 1T, 10AS, 10P, 10Y, 10Z, 10AD, 10AH, 11A, 11B, and 11C; (81) 1T, 10AS, 10P, 10Y, 10AC, 10AG, 10AH, 11A, 11B, and 11C; (82) 1T, 10AS, 10P, 10AB, 10V, 10AH, 11A, 11B, and 11C; (83) 1T, 10AS, 10P, 10AB, 10AF, 10AG, 10AH, 11A, 11B, and 11C; (84) 1T, 10AS, 10P, 10AB, 10W, 11A, 11B, and 11C; (85) 1T, 10AS, 10T, 10R, 10AD, 10AH, 11D, and 11C; (86) 1T, 10AS, 10E, 10F, 10R, 10AD, 10AH, 11D, and 11C; (87) 1T, 10AS, 10E, 10Q, 10Z, 10AD, 10AH, 11D, and 11C; (88) 1T, 10AS, 10E, 10Q, 10AC, 10AG, 10AH, 11D, and 11C; (89) 1T, 10AS, 10P, 10N, 10AD, 10AH, 11D, and 11C; (90) 1T, 10AS, 10P, 10Y, 10Z, 10AD, 10AH, 11D, and 11C; (91) 1T, 10AS, 10P, 10Y, 10AC, 10AG, 10AH, 11D, and 11C; (92) 1T, 10AS, 10P, 10AB, 10V, 10AH, 11D, and 11C; (93) 1T, 10AS, 10P, 10AB, 10AF, 10AG, 10AH, 11D, and 11C; (94) 1T, 10AS, 10P, 10AB, 10W, 11D, and 11C; (95) 10AT, 10T, 10R, 10AD, 10AH, 11A, 11B, and 11C; (96) 10AT, 10E, 10F, 10R, 10AD, 10AH, 11A, 11B, and 11C; (97) 10AT, 10E, 10Q, 10Z, 10AD, 10AH, 11A, 11B, and 11C; (98) 10AT, 10E, 10Q, 10AC, 10AG, 10AH, 11A, 11B, and 11C; (99) 10AT, 10P, 10N, 10AD, 10AH, 11A, 11B, and 11C; (100) 10AT, 10P, 10Y, 10Z, 10AD, 10AH, 11A, 11B, and 11C; (101) 10AT, 10P, 10Y, 10AC, 10AG, 10AH, 11A, 11B, and 11C; (102) 10AT, lop, 10AB, 10V, 10AH, 11A, 11B, and 11C; (103) 10AT, 10P, 10AB, 10AF, 10AG, 10AH, 11A, 11B, and 11C; (104) 10AT, 10P, 10AB, 10W, 11A, 11B, and 11C; (105) 10AT, 10T, 10R, 10AD, 10AH, 11D, and 11C; (106) 10AT, 10E, 10F, 10R, 10AD, 10AH, 11D, and 11C; (107) 10AT, 10E, 10Q, 10Z, 10AD, 10AH, 11D, and 11C; (108) 10AT, 10E, 10Q, 10AC, 10AG, 10AH, 11D, and 11C; (109) 10AT, 10P, 10N, 10AD, 10AH, 11D, and 11C; (110) 10AT, 10P, 10Y, 10Z, 10AD, 10AH, 11D, and 11C; (111) 10AT, 10P, 10Y, 10AC, 10AG, 10AH, 11D, and 10C; (112) 10AT, 10P, 10AB, 10V, 10AH, 11D, and 11C; (113) 10AT, 10P, 10AB, 10AF, 10AG, 10AH, 11D, and 11C; (114) 10AT, 10P, 10AB, 10W, 11D, and 11C; (115) 10AU, 10AF, 10AG, 10AH, 11A, 11B, and 11C; (116) 10AU, 10W, 11A, 11B, and 11C; (117) 10AU, by, 10AH, 11A, 11B, and 11C; (118) 10AU, 10AF, 10AG, 10AH, 11D, and 11C; (119) 10AU, 10W, 11D, and 11C; (120) 10AU, by, 10AH, 11D, and 11C; (121) 10A, 10J, 10R, 10AD, 10AH, and 11E; (122) 10A, 10H, 10F, 10R, 10AD, 10AH, and 11E; (123) 10A, 10H, 10Q, 10Z, 10AD, 10AH, and 11E; (124) 10A, 10H, 10Q, 10AC, 10AG, 10AH, and 11E; (125) 10A, 10D, 10T, 10R, 10AD, 10AH, and 11E; (126) 10A, 10D, 10E, 10F, 10R, 10AD, 10AH, and 11E; (127) 10A, 10D, 10E, 10Q, 10Z, 10AD, 10AH, and 11E; (128) 10A, 10D, 10E, 10Q, 10AC, 10AG, 10AH, and 11E; (129) 10A, 10D, 10P, 10N, 10AD, 10AH, and 11E; (130) 10A, 10D, 10P, 10Y, 10Z, 10AD, 10AH, and 11E; (131) 10A, 10D, 10P, 10Y, 10AC, 10AG, 10AH, and 11E; (132) 10A, 10D, 10P, 10AB, 10V, 10AH, and 11E; (133) 10A, 10D, 10P, 10AB, 10AF, 10AG, 10AH, and 11E; (134) 10A, 10B, 10M, 10AD, 10AH, and 11E; (135) 10A, 10B, 10L, 10Z, 10AD, 10AH, and 11E; (136) 10A, 10B, 10L, 10AC, 10AG, 10AH, and 11E; (137) 10A, 10B, 10X, 10Y, 10Z, 10AD, 10AH, and 11E; (138) 10A, 10B, 10X, 10Y, 10AC, 10AG, 10AH, and 11E; (139) 10A, 10B, 10X, 10AB, 10V, 10AH, and 11E; (140) 10A, 10B, 10X, 10AB, 10AF, 10AG, 10AH, and 11E; (141) 10A, 10B, 10C, 10U, 10AH, and 11E; (142) 10A, 10B, 10C, 10T, 10AG, 10AH, and 11E; (143) 10A, 10B, 10C, 10AE, 10AF, 10AG, 10AH, and 11E; (144) 10A, 10D, 10P, 10AB, 10W, and 11E; (145) 10A, 10B, 10X, 10AB, 10W, and 11E; (146) 10A, 10B, 10C, 10AE, 10W, and 11E; (147) 10A, 10B, 10C, 10AE, by, 10AH, and 11E; (148) 10T, 10R, 10AD, 10AH, and 11E; (149) 10E, 10F, 10R, 10AD, 10AH, and 11E; (150) 10E, 10Q, 10Z, 10AD, 10AH, and 11E; (151) 10E, 10Q, 10AC, 10AG, 10AH, and 11E; (152) 10P, 10N, 10AD, 10AH, and 11E; (153) 10P, 10Y, 10Z, 10AD, 10AH, and 11E; (154) 10P, 10Y, 10AC, 10AG, 10AH, and 11E; (155) 10P, 10AB, 10V, 10AH, and 11E; (156) 10P, 10AB, 10AF, 10AG, 10AH, and 11E; (157) 10P, 10AB, 10W, and 11E; (158) 1T, 10AS, 10T, 10R, 10AD, 10AH, and 11E; (159) 1T, 10AS, 10E, 10F, 10R, 10AD, 10AH, and 11E; (160) 1T, 10AS, 10E, 10Q, 10Z, 10AD, 10AH, and 11E; (161) 1T, 10AS, 10E, 10Q, 10AC, 10AG, 10AH, and 11E; (162) 1T, 10AS, 10P, 10N, 10AD, 10AH, and 11E; (163) 1T, 10AS, 10P, 10Y, 10Z, 10AD, 10AH, and 11E; (164) 1T, 10AS, 10P, 10Y, 10AC, 10AG, 10AH, and 11E; (165) 1T, 10AS, 10P, 10AB, 10V, 10AH, and 11E; (166) 1T, 10AS, 10P, 10AB, 10AF, 10AG, 10AH, and 11E; (167) 1T, 10AS, 10P, 10AB, 10W, and 11E; (168) 10AT, 10T, 10R, 10AD, 10AH, and 11E; (169) 10AT, 10E, 10F, 10R, 10AD, 10AH, and 11E; (170) 10AT, 10E, 10Q, 10Z, 10AD, 10AH, and 11E; (171) 10AT, 10E, 10Q, 10AC, 10AG, 10AH, and 11E; (172) 10AT, 10P, 10N, 10AD, 10AH, and 11E; (173) 10AT, 10P, 10Y, 10Z, 10AD, 10AH, and 11E; (174) 10AT, 10P, 10Y, 10AC, 10AG, 10AH, and 11E; (175) 10AT, 10P, 10AB, 10V, 10AH, and 11E; (176) 10AT, 10P, 10AB, 10AF, 10AG, 10AH, and 11E; (177) 10AT, 10P, 10AB, 10W, and 11E; (178) 10AU, 10AF, 10AG, 10AH, and 11E; (179) 10AU, 10W, and 11E; (180) 10AU, 10V, 10AH, and 11E; (181) 12A, 12B, 12C, 12D, 12E, 12F, 12G, 12H, and 12T; (182) 12A, 12K, 12M, 12N, 12E, 12F, 12G, 12H, and 12T; (183) 12A, 12K, 12L, 12D, 12E, 12F, 12G, 12H, and 12T; (184) 12A, 12O, 12N, 12E, 12F, 12G, 12H, and 12T; (185) 12A, 12B, 12J, 12E, 12F, 12G, 12H, and 12T; (186) 10A, 10D, 10E, 10F, 10G, 10S, 15A, 15B, 15C, and 15G; (187) 10A, 10D, 10T, 10G, 10S, 15A, 15B, 15C, and 15G; (188) 10A, 10D, 10K, 10S, 15A, 15B, 15C, and 15G; (189) 10A, 10H, 10F, 10G, 10S, 15A, 15B, 15C, and 15G; (190) 10A, 10J, 10G, 10S, 15A, 15B, 15C, and 15G; (191) 10A, 10J, 10R, 10AA, 15A, 15B, 15C, and 15G; (192) 10A, 10H, 10F, 10R, 10AA, 15A, 15B, 15C, and 15G; (193) 10A, 10H, 10Q, 10Z, 10AA, 15A, 15B, 15C, and 15G; (194) 10A, 10D, 10I, 10R, 10AA, 15A, 15B, 15C, and 15G; (195) 10A, 10D, 10E, 10F, 10R, 10AA, 15A, 15B, 15C, and 15G; (196) 10A, 10D, 10E, 10Q, 10Z, 10AA, 15A, 15B, 15C, and 15G; (197) 10A, 10D, 10P, 10N, 10AA, 15A, 15B, 15C, and 15G; (198) 10A, 10D, 10P, 10Y, 10Z, 10AA, 15A, 15B, 15C, and 15G; (199) 10A, 10B, 10M, 10AA, 15A, 15B, 15C, and 15G; (200) 10A, 10B, 10L, 10Z, 10AA, 15A, 15B, 15C, and 15G; (201) 10A, 10B, 10X, 10N, 10AA, 15A, 15B, 15C, and 15G; (202) 10A, 10B, 10X, 10Y, 10Z, 10AA, 15A, 15B, 15C, and 15G; (203) 10A, 10D, 10P, 10O, 15A, 15B, 15C, and 15G; (204) 10A, 10B, 10X, 10O, 15A, 15B, 15C, and 15G; (205) 10A, 10D, 10E, 10F, 10R, 10AA, 15A, 15B, 15C, and 15G; (206) 10A, 10D, 10E, 10F, 10G, 10S, 15A, 15B, 15C, and 15G; (207) 10A, 10B, 10C, 10AE, 10AB, 10Y, 10Z, 10AA, 15A, 15B, 15C, and 15G; (208) 10A, 10B, 10C, 10AE, 10AB, 10N, 10AA, 15A, 15B, 15C, and 15G; (209) 10A, 10B, 10C, 10AE, 10AB, 10O, 15A, 15B, 15C, and 15G; (210) 10AU, 10AB, 10Y, 10Z, 10AA, 15A, 15B, 15C, and 15G; (211) 10AU, 10AB, 10N, 10AA, 15A, 15B, 15C, and 15G; (212) 10AU, 10AB, 10O, 15A, 15B, 15C, and 15G; (213) 1T, 10AS, 10E, 10F, 10G, 10S, 15A, 15B, 15C, and 15G; (214) 1T, 10AS, 10I, 10G, 10S, 15A, 15B, 15C, and 15G; (215) 1T, 10AS, 10K, 10S, 15A, 15B, 15C, and 15G; (216) 1T, 10AS, 10I, 10R, 10AA, 15A, 15B, 15C, and 15G; (217) 1T, 10AS, 10E, 10F, 10R, 10AA, 15A, 15B, 15C, and 15G; (218) 1T, 10AS, 10E, 10Q, 10Z, 10AA, 15A, 15B, 15C, and 15G; (219) 1T, 10AS, 10P, 10N, 10AA, 15A, 15B, 15C, and 15G; (220) 1T, 10AS, 10P, 10Y, 10Z, 10AA, 15A, 15B, 15C, and 15G; (221) 1T, 10AS, 10P, 10O, 15A, 15B, 15C, and 15G; (222) 1T, 10AS, 10E, 10F, 10R, 10AA, 15A, 15B, 15C, and 15G; (223) 10AT, 10E, 10F, 10G, 10S, 15A, 15B, 15C, and 15G; (224) 10AT, 10I, 10G, 10S, 15A, 15B, 15C, and 15G; (225) 10AT, 10K, 10S, 15A, 15B, 15C, and 15G; (226) 10AT, 10I, 10R, 10AA, 15A, 15B, 15C, and 15G; (227) 10AT, 10E, 10F, 10R, 10AA, 15A, 15B, 15C, and 15G; (228) 10AT, 10E, 10Q, 10Z, 10AA, 15A, 15B, 15C, and 15G; (229) 10AT, 10P, 10N, 10AA, 15A, 15B, 15C, and 15G; (230) 10AT, 10P, 10Y, 10Z, 10AA, 15A, 15B, 15C, and 15G; (231) 10AT, 10P, 10O, 15A, 15B, 15C, and 15G; (232) 10AT, 10E, 10F, 10R, 10AA, 15A, 15B, 15C, and 15G; (233) 10A, 10D, 10E, 10F, 10G, 10S, 15D, and 15G; (234) 10A, 10D, 10I, 10G, 10S, 15D, and 15G; (235) 10A, 10D, 10K, 10S, 15D, and 15G; (236) 10A, 10H, 10F, 10G, 10S, 15D, and 15G; (237) 10A, 10J, 10G, 10S, 15D, and 15G; (238) 10A, 10J, 10R, 10AA, 15D, and 15G; (239) 10A, 10H, 10F, 10R, 10AA, 15D, and 15G; (240) 10A, 10H, 10Q, 10Z, 10AA, 15D, and 15G; (241) 10A, 10D, 10I, 10R, 10AA, 15D, and 15G; (242) 10A, 10D, 10E, 10F, 10R, 10AA, 15D, and 15G; (243) 10A, 10D, 10E, 10Q, 10Z, 10AA, 15D, and 15G; (244) 10A, 10D, 10P, 10N, 10AA, 15D, and 15G; (245) 10A, 10D, 10P, 10Y, 10Z, 10AA, 15D, and 15G; (246) 10A, 10B, 10M, 10AA, 15D, and 15G; (247) 10A, 10B, 10L, 10Z, 10AA, 15D, and 15G; (248) 10A, 10B, 10X, 10N, 10AA, 15D, and 15G; (249) 10A, 10B, 10X, 10Y, 10Z, 10AA, 15D, and 15G; (250) 10A, 10D, 10P, 10O, 15D, and 15G; (251) 10A, 10B, 10X, 10O, 15D, and 15G; (252) 10A, 10D, 10E, 10F, 10R, 10AA, 15D, and 15G; (253) 10A, 10D, 10E, 10F, 10G, 10S, 15D, and 15G; (254) 10A, 10B, 10C, 10AE, 10AB, 10Y, 10Z, 10AA, 15D, and 15G; (255) 10A, 10B, 10C, 10AE, 10AB, 10N, 10AA, 15D, and 15G; (256) 10A, 10B, 10C, 10AE, 10AB, 10O, 15D, and 15G; (257) 10AU, 10AB, 10Y, 10Z, 10AA, 15D, and 15G; (258) 10AU, 10AB, 10N, 10AA, 15D, and 15G; (259) 10AU, 10AB, 10O, 15D, and 15G; (260) 1T, 10AS, 10E, 10F, 10G, 10S, 15D, and 15G; (261) 1T, 10AS, 10I, 10G, 10S, 15D, and 15G; (262) 1T, 10AS, 10K, 10S, 15D, and 15G; (263) 1T, 10AS, 10I, 10R, 10AA, 15D, and 15G; (264) 1T, 10AS, 10E, 10F, 10R, 10AA, 15D, and 15G; (265) 1T, 10AS, 10E, 10Q, 10Z, 10AA, 15D, and 15G; (266) 1T, 10AS, 10P, 10N, 10AA, 15D, and 15G; (267) 1T, 10AS, 10P, 10Y, 10Z, 10AA, 15D, and 15G; (268) 1T, 10AS, 10P, 10O, 15D, and 15G; (269) 1T, 10AS, 10E, 10F, 10R, 10AA, 15D, and 15G; (270) 10AT, 10E, 10F, 10G, 10S, 15D, and 15G; (271) 10AT, 10I, 10G, 10S, 15D, and 15G; (272) 10AT, 10K, 10S, 15D, and 15G; (273) 10AT, 10I, 10R, 10AA, 15D, and 15G; (274) 10AT, 10E, 10F, 10R, 10AA, 15D, and 15G; (275) 10AT, 10E, 10Q, 10Z, 10AA, 15D, and 15G; (276) 10AT, 10P, 10N, 10AA, 15D, and 15G; (277) 10AT, 10P, 10Y, 10Z, 10AA, 15D, and 15G; (278) 10AT, 10P, 10O, 15D, and 15G; (279) 10AT, 10E, 10F, 10R, 10AA, 15D, and 15G; (280) 10A, 10D, 10E, 10F, 10G, 10S, 15E, 15C, and 15G; (281) 10A, 10D, 10I, 10G, 10S, 15E, 15C, and 15G; (282) 10A, 10D, 10K, 10S, 15E, 15C, and 15G; (283) 10A, 10H, 10F, 10G, 10S, 15E, 15C, and 15G; (284) 10A, 10J, 10G, 10S, 15E, 15C, and 15G; (285) 10A, 10J, 10R, 10AA, 15E, 15C, and 15G; (286) 10A, 10H, 10F, 10R, 10AA, 15E, 15C, and 15G; (287) 10A, 10H, 10Q, 10Z, 10AA, 15E, 15C, and 15G; (288) 10A, 10D, 10I, 10R, 10AA, 15E, 15C, and 15G; (289) 10A, 10D, 10E, 10F, 10R, 10AA, 15E, 15C, and 15G; (290) 10A, 10D, 10E, 10Q, 10Z, 10AA, 15E, 15C, and 15G; (291) 10A, 10D, 10P, 10N, 10AA, 15E, 15C, and 15G; (292) 10A, 10D, 10P, 10Y, 10Z, 10AA, 15E, 15C, and 15G; (293) 10A, 10B, 10M, 10AA, 15E, 15C, and 15G; (294) 10A, 10B, 10L, 10Z, 10AA, 15E, 15C, and 15G; (295) 10A, 10B, 10X, 10N, 10AA, 15E, 15C, and 15G; (296) 10A, 10B, 10X, 10Y, 10Z, 10AA, 15E, 15C, and 15G; (297) 10A, 10D, 10P, 10O, 15E, 15C, and 15G; (298) 10A, 10B, 10X, 10O, 15E, 15C, and 15G; (299) 10A, 10D, 10E, 10F, 10R, 10AA, 15E, 15C, and 15G; (300) 10A, 10D, 10E, 10F, 10G, 10S, 15E, 15C, and 15G; (301) 10A, 10B, 10C, 10AE, 10AB, 10Y, 10Z, 10AA, 15E, 15C, and 15G; (302) 10A, 10B, 10C, 10AE, 10AB, 10N, 10AA, 15E, 15C, and 15G; (303) 10A, 10B, 10C, 10AE, 10AB, 10O, 15E, 15C, and 15G; (304) 10AU, 10AB, 10Y, 10Z, 10AA, 15E, 15C, and 15G; (305) 10AU, 10AB, 10N, 10AA, 15E, 15C, and 15G; (306) 10AU, 10AB, 10O, 15E, 15C, and 15G; (307) 1T, 10AS, 10E, 10F, 10G, 10S, 15E, 15C, and 15G; (308) 1T, 10AS, 10I, 10G, 10S, 15E, 15C, and 15G; (309) 1T, 10AS, 10K, 10S, 15E, 15C, and 15G; (310) 1T, 10AS, 10I, 10R, 10AA, 15E, 15C, and 15G; (311) 1T, 10AS, 10E, 10F, 10R, 10AA, 15E, 15C, and 15G; (312) 1T, 10AS, 10E, 10Q, 10Z, 10AA, 15E, 15C, and 15G; (313) 1T, 10AS, 10P, 10N, 10AA, 15E, 15C, and 15G; (314) 1T, 10AS, 10P, 10Y, 10Z, 10AA, 15E, 15C, and 15G; (315) 1T, 10AS, 10P, 10O, 15E, 15C, and 15G; (316) 1T, 10AS, 10E, 10F, 10R, 10AA, 15E, 15C, and 15G; (317) 10AT, 10E, 10F, 10G, 10S, 15E, 15C, and 15G; (318) 10AT, 10I, 10G, 10S, 15E, 15C, and 15G; (319) 10AT, 10K, 10S, 15E, 15C, and 15G; (320) 10AT, 10I, 10R, 10AA, 15E, 15C, and 15G; (321) 10AT, 10E, 10F, 10R, 10AA, 15E, 15C, and 15G; (322) 10AT, 10E, 10Q, 10Z, 10AA, 15E, 15C, and 15G; (323) 10AT, 10P, 10N, 10AA, 15E, 15C, and 15G; (324) 10AT, 10P, 10Y, 10Z, 10AA, 15E, 15C, and 15G; (325) 10AT, 10P, 10O, 15E, 15C, and 15G; (326) 10AT, 10E, 10F, 10R, 10AA, 15E, 15C, and 15G; (327) 10A, 10D, 10E, 10F, 10G, 10S, 15A, 15F, and 15G; (328) 10A, 10D, 10I, 10G, 10S, 15A, 15F, and 15G; (329) 10A, 10D, 10K, 10S, 15A, 15F, and 15G; (330) 10A, 10H, 10F, 10G, 10S, 15A, 15F, and 15G; (331) 10A, 10J, 10G, 10S, 15A, 15F, and 15G; (332) 10A, 10J, 10R, 10AA, 15A, 15F, and 15G; (333) 10A, 10H, 10F, 10R, 10AA, 15A, 15F, and 15G; (334) 10A, 10H, 10Q, 10Z, 10AA, 15A, 15F, and 15G; (335) 10A, 10D, 10I, 10R, 10AA, 15A, 15F, and 15G; (336) 10A, 10D, 10E, 10F, 10R, 10AA, 15A, 15F, and 15G; (337) 10A, 10D, 10E, 10Q, 10Z, 10AA, 15A, 15F, and 15G; (338) 10A, 10D, 10P, 10N, 10AA, 15A, 15F, and 15G; (339) 10A, 10D, 10P, 10Y, 10Z, 10AA, 15A, 15F, and 15G; (340) 10A, 10B, 10M, 10AA, 15A, 15F, and 15G; (341) 10A, 10B, 10L, 10Z, 10AA, 15A, 15F, and 15G; (342) 10A, 10B, 10X, 10N, 10AA, 15A, 15F, and 15G; (343) 10A, 10B, 10X, 10Y, 10Z, 10AA, 15A, 15F, and 15G; (344) 10A, 10D, 10P, 10O, 15A, 15F, and 15G; (345) 10A, 10B, 10X, 10O, 15A, 15F, and 15G; (346) 10A, 10D, 10E, 10F, 10R, 10AA, 15A, 15F, and 15G; (347) 10A, 10D, 10E, 10F, 10G, 10S, 15A, 15F, and 15G; (348) 10A, 10B, 10C, 10AE, 10AB, 10Y, 10Z, 10AA, 15A, 15F, and 15G; (349) 10A, 10B, 10C, 10AE, 10AB, 10N, 10AA, 15A, 15F, and 15G; (350) 10A, 10B, 10C, 10AE, 10AB, 10O, 15A, 15F, and 15G; (351) 10AU, 10AB, 10Y, 10Z, 10AA, 15A, 15F, and 15G; (352) 10AU, 10AB, 10N, 10AA, 15A, 15F, and 15G; (353) 10AU, 10AB, 10O, 15A, 15F, and 15G; (354) 1T, 10AS, 10E, 10F, 10G, 10S, 15A, 15F, and 15G; (355) 1T, 10AS, 10I, 10G, 10S, 15A, 15F, and 15G; (356) 1T, 10AS, 10K, 10S, 15A, 15F, and 15G; (357) 1T, 10AS, 10I, 10R, 10AA, 15A, 15F, and 15G; (358) 1T, 10AS, 10E, 10F, 10R, 10AA, 15A, 15F, and 15G; (359) 1T, 10AS, 10E, 10Q, 10Z, 10AA, 15A, 15F, and 15G; (360) 1T, 10AS, 10P, 10N, 10AA, 15A, 15F, and 15G; (361) 1T, 10AS, 10P, 10Y, 10Z, 10AA, 15A, 15F, and 15G; (362) 1T, 10AS, 10P, 10O, 15A, 15F, and 15G; (363) 1T, 10AS, 10E, 10F, 10R, 10AA, 15A, 15F, and 15G; (364) 10AT, 10E, 10F, 10G, 10S, 15A, 15F, and 15G; (365) 10AT, 10I, 10G, 10S, 15A, 15F, and 15G; (366) 10AT, 10K, 10S, 15A, 15F, and 15G; (367) 10AT, 10P, 10R, 10AA, 15A, 15F, and 15G; (368) 10AT, 10E, 10F, 10R, 10AA, 15A, 15F, and 15G; (369) 10AT, 10E, 10Q, 10Z, 10AA, 15A, 15F, and 15G; (370) 10AT, 10P, 10N, 10AA, 15A, 15F, and 15G; (371) 10AT, 10P, 10Y, 10Z, 10AA, 15A, 15F, and 15G; (372) 10AT, 10P, 10O, 15A, 15F, and 15G; (373) 10AT, 10E, 10F, 10R, 10AA, 15A, 15F, and 15G; (374) 14A, 14B, 14C, 14D, 14E, 13A, and 13B; (375) 16A, 16B, 16C, 16D, and 16E; (376) 17A, 17B, 17C, 17D, and 17G; (377) 17A, 17E, 17F, 17D, and 17G; (378) 17A, 17B, 17C, 17H, 17I, 17J, and 17G; (379) 18A, 18B, 18C, 18D, 18E, and 18F; (380) 13A and 13B; and (381) 7A, 17E, 17F, 17H, 17I, 17J, and 17G, wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10T is a crotonyl-ACP thioesterase, wherein 10U is a crotonyl-ACP reductase (aldehyde forming), wherein 10V is a crotonyl-CoA reductase (aldehyde forming), wherein 10W is a crotonyl-CoA (alcohol forming), wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AC is a 3-hydroxybutyrate dehydratase, wherein LOAD is a 3-hydroxybutyraldehyde dehydratase, wherein 10AE is a crotonyl-CoA:ACP transferase, wherein 10AF is a crotonyl-CoA hydrolase, transferase or synthetase, wherein 10AG is a crotonate reductase, wherein 10AH is a crotonaldehyde reductase, wherein 10AS is an acetoacetyl-CoA synthase, wherein 10AT is an acetyl-CoA:acetyl-CoA acyltransferase, wherein 10AU is a 4-hydroxybutyryl-CoA dehydratase, wherein 11A is a crotyl alcohol kinase, wherein 11B is a 2-butenyl-4-phosphate kinase, wherein 11C is a butadiene synthase, wherein 11D is a crotyl alcohol diphosphokinase, wherein 11E is a crotyl alcohol dehydratase, wherein 12A is a malonyl-CoA:acetyl-CoA acyltransferase, wherein 12B is a 3-oxoglutaryl-CoA reductase (ketone-reducing), wherein 12C is a 3-hydroxyglutaryl-CoA reductase (aldehyde forming), wherein 12D is a 3-hydroxy-5-oxopentanoate reductase, wherein 12E is a 3,5-dihydroxypentanoate kinase, wherein 12F is a 3-hydroxy-5-phosphonatooxypentanoate kinase, wherein 12G is a 3-hydroxy-5-[hydroxy(phosphonooxy)phosphoryl]oxy pentanoate decarboxylase, wherein 12H is a butenyl 4-diphosphate isomerase, wherein 12I is a butadiene synthase, wherein 12J is a 3-hydroxyglutaryl-CoA reductase (alcohol forming), wherein 12K is a 3-oxoglutaryl-CoA reductase (aldehyde forming), wherein 12L is a 3,5-dioxopentanoate reductase (ketone reducing), wherein 12M is a 3,5-dioxopentanoate reductase (aldehyde reducing), wherein 12N is a 5-hydroxy-3-oxopentanoate reductase, wherein 12O is a 3-oxo-glutaryl-CoA reductase (CoA reducing and alcohol forming), wherein 13A is a 2-butanol desaturase, wherein 13B is a 3-buten-2-ol dehydratase, wherein 14A is an acetolactate synthase, wherein 14B is an acetolactate decarboxylase, wherein 14C is a butanediol dehydrogenase, wherein 14D is a butanediol dehydratase, wherein 14E is a butanol dehydrogenase, wherein 15A is a 1,3-butanediol kinase, wherein 15B is a 3-hydroxybutyrylphosphate kinase, 15C is a 3-hydroxybutyryldiphosphate lyase, wherein 15D is a 1,3-butanediol diphosphokinase, wherein 15E is a 1,3-butanediol dehydratase, wherein 15F is a 3-hydroxybutyrylphosphate lyase, wherein 15G is a 3-buten-2-ol dehydratase, wherein 16A is a 3-oxopent-4-enoyl-CoA thiolase, wherein 16B is a 3-oxopent-4-enoyl-CoA hydrolase, synthetase or transferase, wherein 16C is a 3-oxopent-4-enoate decarboxylase or spontaneous, wherein 16D is a 3-buten-2-one reductase, wherein 16E is a 3-buten-2-ol dehydratase, wherein 17A is a 3-oxo-4-hydroxypentanoyl-CoA thiolase, wherein 17B is a 3-oxo-4-hydroxypentanoyl-CoA transferase, synthetase or hydrolase, wherein 17C is a 3-oxo-4-hydroxypentanoate reductase, wherein 17D is a 3,4-dihydroxypentanoate decarboxylase, wherein 17E is a 3-oxo-4-hydroxypentanoyl-CoA reductase, wherein 17F is a 3,4-dihydroxypentanoyl-CoA transferase, synthetase or hydrolase, wherein 17G is a 3-buten-2-ol dehydratase, wherein 17H is a 3,4-dihydroxypentanoate dehydratase, wherein 17I is a 4-oxopentanoate reductase, wherein 17J is a 4-hyd4-oxoperoxypentanoate decarboxylase, wherein 18A is a 3-oxoadipyl-CoA thiolase, wherein 18B is a 3-oxoadipyl-CoA transferase, synthetase or hydrolase, wherein 18C is a 3-oxoadipate decarboxylase or spontaneous, wherein 18D is a 4-oxopentanoate reductase, wherein 18E is a 4-hydroxypentanoate decarboxylase, wherein 18F is a 3-buten-2-ol dehydratase.

In certain embodiments, provided herein is a non-naturally occurring microbial organism having a formaldehyde fixation pathway, a formate assimilation pathway, a methanol metabolic pathway, a methanol oxidation pathway, a hydrogenase, a carbon monoxide dehydrogenase or any combination described above, wherein the organism further comprises a crotyl alcohol pathway. In certain embodiments, the microbial organism comprises at least one exogenous nucleic acid encoding a crotyl alcohol pathway enzyme expressed in a sufficient amount to produce crotyl alcohol, wherein said crotyl alcohol pathway comprises a pathway as shown in FIGS. 1, 2, and 10 selected from: (1) 10A, 10J, 10R, 10AD, and 10AH; (2) 10A, 10H, 10F, 10R, 10AD, and 10AH; (3) 10A, 10H, 10Q, 10Z, 10AD, and 10AH; (4) 10A, 10H, 10Q, 10AC, 10AG, and 10AH; (5) 10A, 10D, 10I, 10R, 10AD, and 10AH; (6) 10A, 10D, 10E, 10F, 10R, 10AD, and 10AH; (7) 10A, 10D, 10E, 10Q, 10Z, 10AD, and 10AH; (8) 10A, 10D, 10E, 10Q, 10AC, 10AG, and 10AH; (9) 10A, 10D, 10P, 10N, 10AD, and 10AH; (10) 10A, 10D, 10P, 10Y, 10Z, 10AD, and 10AH; (11) 10A, 10D, 10P, 10Y, 10AC, 10AG, and 10AH; (12) 10A, 10D, 10P, 10AB, 10V, and 10AH; (13) 10A, 10D, 10P, 10AB, 10AF, 10AG, and 10AH; (14) 10A, 10B, 10M, 10AD, and 10AH; (15) 10A, 10B, 10L, 10Z, 10AD, and 10AH; (16) 10A, 10B, 10L, 10AC, 10AG, and 10AH; (17) 10A, 10B, 10X, 10Y, 10Z, 10AD, and 10AH; (18) 10A, 10B, 10X, 10Y, 10AC, 10AG, and 10AH; (19) 10A, 10B, 10X, 10AB, 10V, and 10AH; (20) 10A, 10B, 10X, 10AB, 10AF, 10AG, and 10AH; (21) 10A, 10B, 10C, 10U, and 10AH; (22) 10A, 10B, 10C, 10T, 10AG, and 10AH; (23) 10A, 10B, 10C, 10AE, 10AF, 10AG, and 10AH; (24) 10A, 10D, 10P, 10AB, and 10W; (25) 10A, 10B, 10X, 10AB, and 10W; (26) 10A, 10B, 10C, 10AE, and 10W; (27) 10A, 10B, 10C, 10AE, 10V, and 10AH; (28) 10I, 10R, 10AD, and 10AH; (29) 10E, 10F, 10R, 10AD, and 10AH; (30) 10E, 10Q, 10Z, 10AD, and 10AH; (31) 10E, 10Q, 10AC, 10AG, and 10AH; (32) 10P, 10N, 10AD, and 10AH; (33) 10P, 10Y, 10Z, 10AD, and 10AH; (34) 10P, 10Y, 10AC, 10AG, and 10AH; (35) 10P, 10AB, 10V, and 10AH; (36) 10P, 10AB, 10AF, 10AG, and 10AH; (37) 10P, 10AB, and 10W; (38) 1T, 10AS, 10I, 10R, 10AD, and 10AH; (39) 1T, 10AS, 10E, 10F, 10R, 10AD, and 10AH; (40) 1T, 10AS, 10E, 10Q, 10Z, 10AD, and 10AH; (41) 1T, 10AS, 10E, 10Q, 10AC, 10AG, and 10AH; (42) 1T, 10AS, 10P, 10N, 10AD, and 10AH; (43) 1T, 10AS, 10P, 10Y, 10Z, 10AD, and 10AH; (44) 1T, 10AS, 10P, 10Y, 10AC, 10AG, and 10AH; (45) 1T, 10AS, 10P, 10AB, 10V, and 10AH; (46) 1T, 10AS, 10P, 10AB, 10AF, 10AG, and 10AH; (47) 1T, 10AS, 10P, 10AB, and 10W; (48) 10AT, 10I, 10R, 10AD, and 10AH; (49) 10AT, 10E, 10F, 10R, 10AD, and 10AH; (50) 10AT, 10E, 10Q, 10Z, 10AD, and 10AH; (51) 10AT, 10E, 10Q, 10AC, 10AG, and 10AH; (52) 10AT, 10P, 10N, 10AD, and 10AH; (53) 10AT, 10P, 10Y, 10Z, 10AD, and 10AH; (54) 10AT, 10P, 10Y, 10AC, 10AG, and 10AH; (55) 10AT, 10P, 10AB, 10V, and 10AH; (56) 10AT, 10P, 10AB, 10AF, 10AG, and 10AH; (57) 10AT, 10P, 10AB, and 10W; (58) 10AU, 10AF, 10AG, and 10AH; (59) 10AU, and 10W; and (60) 10AU, 10V, and 10AH, wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10T is a crotonyl-ACP thioesterase, wherein 10U is a crotonyl-ACP reductase (aldehyde forming), wherein 10V is a crotonyl-CoA reductase (aldehyde forming), wherein 10W is a crotonyl-CoA (alcohol forming), wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AB is a 3-hydroxybutyryl-CoA dehydratase, wherein 10AC is a 3-hydroxybutyrate dehydratase, wherein LOAD is a 3-hydroxybutyraldehyde dehydratase, wherein 10AE is a crotonyl-CoA:ACP transferase, wherein LOAF is a crotonyl-CoA hydrolase, transferase or synthetase, wherein 10AG is a crotonate reductase, wherein 10AH is a crotonaldehyde reductase, wherein 10AS is an acetoacetyl-CoA synthase, wherein 10AT is an acetyl-CoA:acetyl-CoA acyltransferase, wherein 10AU is a 4-hydroxybutyryl-CoA dehydratase.

In certain embodiments, provided herein is a non-naturally occurring microbial organism having a formaldehyde fixation pathway, a formate assimilation pathway, a methanol metabolic pathway, a methanol oxidation pathway, a hydrogenase, a carbon monoxide dehydrogenase or any combination described above, wherein the organism further comprises a 1,3-butanediol pathway. In certain embodiments, the microbial organism comprises at least one exogenous nucleic acid encoding a 1,3-butanediol pathway enzyme expressed in a sufficient amount to produce 1,3-butanediol, wherein said 1,3-butanediol pathway comprises a pathway shown in FIGS. 1 and 10 selected from: (1) 10A, 10D, 10E, 10F, 10G, and 10S; (2) 10A, 10D, 10I, 10G, and 10S; (3) 10A, 10D, 10K, and 10S; (4) 10A, 10H, 10F, 10G, and 10S; (5) 10A, 10J, 10G, and 10S; (6) 10A, 10J, 10R, and 10AA; (7) 10A, 10H, 10F, 10R, and 10AA; (8) 10A, 10H, 10Q, 10Z, and 10AA; (9) 10A, 10D, 10I, 10R, and 10AA; (10) 10A, 10D, 10E, 10F, 10R, and 10AA; (11) 10A, 10D, 10E, 10Q, 10Z, and 10AA; (12) 10A, 10D, 10P, 10N, and 10AA; (13) 10A, 10D, 10P, 10Y, 10Z, and 10AA; (14) 10A, 10B, 10M, and 10AA; (15) 10A, 10B, 10L, 10Z, and 10AA; (16) 10A, 10B, 10X, 10N, and 10AA; (17) 10A, 10B, 10X, 10Y, 10Z, and 10AA; (18) 10A, 10D, 10P, and 10O; (19) 10A, 10B, 10X, and 10O; (20) 10A, 10D, 10E, 10F, 10R, and 10AA; (21) 10A, 10D, 10E, 10F, 10G, and 10S; (22) 10A, 10B, 10C, 10AE, 10AB, 10Y, 10Z, and 10AA; (23) 10A, 10B, 10C, 10AE, 10AB, 10N, and 10AA; (24) 10A, 10B, 10C, 10AE, 10AB, and 10O; (25) 10AU, 10AB, 10Y, 10Z, and 10AA; (26) 10AU, 10AB, 10N, and 10AA; (27) 10AU, 10AB, and 10O; (28) 1T, 10AS, 10E, 10F, 10G, and 10S; (29) 1T, 10AS, 10I, 10G, and 10S; (30) 1T, 10AS, 10K, and 10S; (31) 1T, 10AS, 10I, 10R, and 10AA; (32) 1T, 10AS, 10E, 10F, 10R, and 10AA; (33) 1T, 10AS, 10E, 10Q, 10Z, and 10AA; (34) 1T, 10AS, 10P, 10N, and 10AA; (35) 1T, 10AS, 10P, 10Y, 10Z, and 10AA; (36) 1T, 10AS, 10P, and 10O; (37) 1T, 10AS, 10E, 10F, 10R, and 10AA; (38) 10AT, 10E, 10F, 10G, and 10S; (39) 10AT, 10I, 10G, and 10S; (40) 10AT, 10K, and 10S; (41) 10AT, 10I, 10R, and 10AA; (42) 10AT, 10E, 10F, 10R, and 10AA; (43) 10AT, 10E, 10Q, 10Z, and 10AA; (44) 10AT, 10P, 10N, and 10AA; (45) 10AT, 10P, 10Y, 10Z, and 10AA; (46) 10AT, 10P, and 10O; and (47) 10AT, 10E, 10F, 10R, and 10AA, wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AE is a crotonyl-CoA:ACP transferase, wherein 10AS is an acetoacetyl-CoA synthase, wherein 10AT is an acetyl-CoA:acetyl-CoA acyltransferase, wherein 10AU is a 4-hydroxybutyryl-CoA dehydratase.

In some embodiments, the invention provides a non-naturally occurring microbial organism having a 3-buten-2-ol pathway including at least one exogenous nucleic acid encoding a 3-buten-2-ol pathway enzyme expressed in a sufficient amount to produce 3-buten-2-ol, wherein the 3-buten-2-ol pathway includes a pathway shown in FIGS. 1, 10, and 13-18 selected from: (1) 10A, 10D, 10E, 10F, 10G, 10S, 15A, 15B, and 15C; (2) 10A, 10D, 10I, 10G, 10S, 15A, 15B, and 15C; (3) 10A, 10D, 10K, 10S, 15A, 15B, and 15C; (4) 10A, 10H, 10F, 10G, 10S, 15A, 15B, and 15C; (5) 10A, 10J, 10G, 10S, 15A, 15B, and 15C; (6) 10A, 10J, 10R, 10AA, 15A, 15B, and 15C; (7) 10A, 10H, 10F, 10R, 10AA, 15A, 15B, and 15C; (8) 10A, 10H, 10Q, 10Z, 10AA, 15A, 15B, and 15C; (9) 10A, 10D, 10I, 10R, 10AA, 15A, 15B, and 15C; (10) 10A, 10D, 10E, 10F, 10R, 10AA, 15A, 15B, and 15C; (11) 10A, 10D, 10E, 10Q, 10Z, 10AA, 15A, 15B, and 15C; (12) 10A, 10D, 10P, 10N, 10AA, 15A, 15B, and 15C; (13) 10A, 10D, 10P, 10Y, 10Z, 10AA, 15A, 15B, and 15C; (14) 10A, 10B, 10M, 10AA, 15A, 15B, and 15C; (15) 10A, 10B, 10L, 10Z, 10AA, 15A, 15B, and 15C; (16) 10A, 10B, 10X, 10N, 10AA, 15A, 15B, and 15C; (17) 10A, 10B, 10X, 10Y, 10Z, 10AA, 15A, 15B, and 15C; (18) 10A, 10D, 10P, 10O, 15A, 15B, and 15C; (19) 10A, 10B, 10X, 10O, 15A, 15B, and 15C; (20) 10A, 10D, 10E, 10F, 10R, 10AA, 15A, 15B, and 15C; (21) 10A, 10D, 10E, 10F, 10G, 10S, 15A, 15B, and 15C; (22) 10A, 10B, 10C, 10AE, 10AB, 10Y, 10Z, 10AA, 15A, 15B, and 15C; (23) 10A, 10B, 10C, 10AE, 10AB, 10N, 10AA, 15A, 15B, and 15C; (24) 10A, 10B, 10C, 10AE, 10AB, 10O, 15A, 15B, and 15C; (25) 10AU, 10AB, 10Y, 10Z, 10AA, 15A, 15B, and 15C; (26) 10AU, 10AB, 10N, 10AA, 15A, 15B, and 15C; (27) 10AU, 10AB, 10O, 15A, 15B, and 15C; (28) 1T, 10AS, 10E, 10F, 10G, 10S, 15A, 15B, and 15C; (29) 1T, 10AS, 10I, 10G, 10S, 15A, 15B, and 15C; (30) 1T, 10AS, 10K, 10S, 15A, 15B, and 15C; (31) 1T, 10AS, 10I, 10R, 10AA, 15A, 15B, and 15C; (32) 1T, 10AS, 10E, 10F, 10R, 10AA, 15A, 15B, and 15C; (33) 1T, 10AS, 10E, 10Q, 10Z, 10AA, 15A, 15B, and 15C; (34) 1T, 10AS, 10P, 10N, 10AA, 15A, 15B, and 15C; (35) 1T, 10AS, 10P, 10Y, 10Z, 10AA, 15A, 15B, and 15C; (36) 1T, 10AS, 10P, 10O, 15A, 15B, and 15C; (37) 1T, 10AS, 10E, 10F, 10R, 10AA, 15A, 15B, and 15C; (38) 10AT, 10E, 10F, 10G, 10S, 15A, 15B, and 15C; (39) 10AT, 10I, 10G, 10S, 15A, 15B, and 15C; (40) 10AT, 10K, 10S, 15A, 15B, and 15C; (41) 10AT, 10I, 10R, 10AA, 15A, 15B, and 15C; (42) 10AT, 10E, 10F, 10R, 10AA, 15A, 15B, and 15C; (43) 10AT, 10E, 10Q, 10Z, 10AA, 15A, 15B, and 15C; (44) 10AT, 10P, 10N, 10AA, 15A, 15B, and 15C; (45) 10AT, 10P, 10Y, 10Z, 10AA, 15A, 15B, and 15C; (46) 10AT, 10P, 10O, 15A, 15B, and 15C; (47) 10AT, 10E, 10F, 10R, 10AA, 15A, 15B, and 15C; (48) 10A, 10D, 10E, 10F, 10G, 10S, and 15D; (49) 10A, 10D, 10I, 10G, 10S, and 15D; (50) 10A, 10D, 10K, 10S, and 15D; (51) 10A, 10H, 10F, 10G, 10S, and 15D; (52) 10A, 10J, 10G, 10S, and 15D; (53) 10A, 10J, 10R, 10AA, and 15D; (54) 10A, 10H, 10F, 10R, 10AA, and 15D; (55) 10A, 10H, 10Q, 10Z, 10AA, and 15D; (56) 10A, 10D, 10I, 10R, 10AA, and 15D; (57) 10A, 10D, 10E, 10F, 10R, 10AA, and 15D; (58) 10A, 10D, 10E, 10Q, 10Z, 10AA, and 15D; (59) 10A, 10D, 10P, 10N, 10AA, and 15D; (60) 10A, 10D, 10P, 10Y, 10Z, 10AA, and 15D; (61) 10A, 10B, 10M, 10AA, and 15D; (62) 10A, 10B, 10L, 10Z, 10AA, and 15D; (63) 10A, 10B, 10X, 10N, 10AA, and 15D; (64) 10A, 10B, 10X, 10Y, 10Z, 10AA, and 15D; (65) 10A, 10D, 10P, 10O, and 15D; (66) 10A, 10B, 10X, 10O, and 15D; (67) 10A, 10D, 10E, 10F, 10R, 10AA, and 15D; (68) 10A, 10D, 10E, 10F, 10G, 10S, and 15D; (69) 10A, 10B, 10C, 10AE, 10AB, 10Y, 10Z, 10AA, and 15D; (70) 10A, 10B, 10C, 10AE, 10AB, 10N, 10AA, and 15D; (71) 10A, 10B, 10C, 10AE, 10AB, 10O, and 15D; (72) 10AU, 10AB, 10Y, 10Z, 10AA, and 15D; (73) 10AU, 10AB, 10N, 10AA, and 15D; (74) 10AU, 10AB, 10O, and 15D; (75) 1T, 10AS, 10E, 10F, 10G, 10S, and 15D; (76) 1T, 10AS, 10I, 10G, 10S, and 15D; (77) 1T, 10AS, 10K, 10S, and 15D; (78) 1T, 10AS, 10I, 10R, 10AA, and 15D; (79) 1T, 10AS, 10E, 10F, 10R, 10AA, and 15D; (80) 1T, 10AS, 10E, 10Q, 10Z, 10AA, and 15D; (81) 1T, 10AS, 10P, 10N, 10AA, and 15D; (82) 1T, 10AS, 10P, 10Y, 10Z, 10AA, and 15D; (83) 1T, 10AS, 10P, 10O, and 15D; (84) 1T, 10AS, 10E, 10F, 10R, 10AA, and 15D; (85) 10AT, 10E, 10F, 10G, 10S, and 15D; (86) 10AT, 10I, 10G, 10S, and 15D; (87) 10AT, 10K, 10S, and 15D; (88) 10AT, 10I, 10R, 10AA, and 15D; (89) 10AT, 10E, 10F, 10R, 10AA, and 15D; (90) 10AT, 10E, 10Q, 10Z, 10AA, and 15D; (91) 10AT, 10P, 10N, 10AA, and 15D; (92) 10AT, 10P, 10Y, 10Z, 10AA, and 15D; (93) 10AT, 10P, 10O, and 15D; (94) 10AT, 10E, 10F, 10R, 10AA, and 15D; (95) 10A, 10D, 10E, 10F, 10G, 10S, 15E, and 15C; (96) 10A, 10D, 10I, 10G, 10S, 15E, and 15C; (97) 10A, 10D, 10K, 10S, 15E, and 15C; (98) 10A, 10H, 10F, 10G, 10S, 15E, and 15C; (99) 10A, 10J, 10G, 10S, 15E, and 15C; (100) 10A, 10J, 10R, 10AA, 15E, and 15C; (101) 10A, 10H, 10F, 10R, 10AA, 15E, and 15C; (102) 10A, 10H, 10Q, 10Z, 10AA, 15E, and 15C; (103) 10A, 10D, 10I, 10R, 10AA, 15E, and 15C; (104) 10A, 10D, 10E, 10F, 10R, 10AA, 15E, and 15C; (105) 10A, 10D, 10E, 10Q, 10Z, 10AA, 15E, and 15C; (106) 10A, 10D, 10P, 10N, 10AA, 15E, and 15C; (107) 10A, 10D, 10P, 10Y, 10Z, 10AA, 15E, and 15C; (108) 10A, 10B, 10M, 10AA, 15E, and 15C; (109) 10A, 10B, 10L, 10Z, 10AA, 15E, and 15C; (110) 10A, 10B, 10X, 10N, 10AA, 15E, and 15C; (111) 10A, 10B, 10X, 10Y, 10Z, 10AA, 15E, and 15C; (112) 10A, 10D, 10P, 10O, 15E, and 15C; (113) 10A, 10B, 10X, 10O, 15E, and 15C; (114) 10A, 10D, 10E, 10F, 10R, 10AA, 15E, and 15C; (115) 10A, 10D, 10E, 10F, 10G, 10S, 15E, and 15C; (116) 10A, 10B, 10C, 10AE, 10AB, 10Y, 10Z, 10AA, 15E, and 15C; (117) 10A, 10B, 10C, 10AE, 10AB, 10N, 10AA, 15E, and 15C; (118) 10A, 10B, 10C, 10AE, 10AB, 10O, 15E, and 15C; (119) 10AU, 10AB, 10Y, 10Z, 10AA, 15E, and 15C; (120) 10AU, 10AB, 10N, 10AA, 15E, and 15C; (121) 10AU, 10AB, 10O, 15E, and 15C; (122) 1T, 10AS, 10E, 10F, 10G, 10S, 15E, and 15C; (123) 1T, 10AS, 10I, 10G, 10S, 15E, and 15C; (124) 1T, 10AS, 10K, 10S, 15E, and 15C; (125) 1T, 10AS, 10I, 10R, 10AA, 15E, and 15C; (126) 1T, 10AS, 10E, 10F, 10R, 10AA, 15E, and 15C; (127) 1T, 10AS, 10E, 10Q, 10Z, 10AA, 15E, and 15C; (128) 1T, 10AS, 10P, 10N, 10AA, 15E, and 15C; (129) 1T, 10AS, 10P, 10Y, 10Z, 10AA, 15E, and 15C; (130) 1T, 10AS, 10P, 10O, 15E, and 15C; (131) 1T, 10AS, 10E, 10F, 10R, 10AA, 15E, and 15C; (132) 10AT, 10E, 10F, 10G, 10S, 15E, and 15C; (133) 10AT, 10I, 10G, 10S, 15E, and 15C; (134) 10AT, 10K, 10S, 15E, and 15C; (135) 10AT, 10I, 10R, 10AA, 15E, and 15C; (136) 10AT, 10E, 10F, 10R, 10AA, 15E, and 15C; (137) 10AT, 10E, 10Q, 10Z, 10AA, 15E, and 15C; (138) 10AT, 10P, 10N, 10AA, 15E, and 15C; (139) 10AT, 10P, 10Y, 10Z, 10AA, 15E, and 15C; (140) 10AT, 10P, 10O, 15E, and 15C; (141) 10AT, 10E, 10F, 10R, 10AA, 15E, and 15C; (142) 10A, 10D, 10E, 10F, 10G, 10S, 15A, and 15F; (143) 10A, 10D, 10I, 10G, 10S, 15A, and 15F; (144) 10A, 10D, 10K, 10S, 15A, and 15F; (145) 10A, 10H, 10F, 10G, 10S, 15A, and 15F; (146) 10A, 10J, 10G, 10S, 15A, and 15F; (147) 10A, 10J, 10R, 10AA, 15A, and 15F; (148) 10A, 10H, 10F, 10R, 10AA, 15A, and 15F; (149) 10A, 10H, 10Q, 10Z, 10AA, 15A, and 15F; (150) 10A, 10D, 10I, 10R, 10AA, 15A, and 15F; (151) 10A, 10D, 10E, 10F, 10R, 10AA, 15A, and 15F; (152) 10A, 10D, 10E, 10Q, 10Z, 10AA, 15A, and 15F; (153) 10A, 10D, 10P, 10N, 10AA, 15A, and 15F; (154) 10A, 10D, 10P, 10Y, 10Z, 10AA, 15A, and 15F; (155) 10A, 10B, 10M, 10AA, 15A, and 15F; (156) 10A, 10B, 10L, 10Z, 10AA, 15A, and 15F; (157) 10A, 10B, 10X, 10N, 10AA, 15A, and 15F; (158) 10A, 10B, 10X, 10Y, 10Z, 10AA, 15A, and 15F; (159) 10A, 10D, 10P, 10O, 15A, and 15F; (160) 10A, 10B, 10X, 10O, 15A, and 15F; (161) 10A, 10D, 10E, 10F, 10R, 10AA, 15A, and 15F; (162) 10A, 10D, 10E, 10F, 10G, 10S, 15A, and 15F; (163) 10A, 10B, 10C, 10AE, 10AB, 10Y, 10Z, 10AA, 15A, and 15F; (164) 10A, 10B, 10C, 10AE, 10AB, 10N, 10AA, 15A, and 15F; (165) 10A, 10B, 10C, 10AE, 10AB, 10O, 15A, and 15F; (166) 10AU, 10AB, 10Y, 10Z, 10AA, 15A, and 15F; (167) 10AU, 10AB, 10N, 10AA, 15A, and 15F; (168) 10AU, 10AB, 10O, 15A, and 15F; (169) 1T, 10AS, 10E, 10F, 10G, 10S, 15A, and 15F; (170) 1T, 10AS, 10I, 10G, 10S, 15A, and 15F; (171) 1T, 10AS, 10K, 10S, 15A, and 15F; (172) 1T, 10AS, 10I, 10R, 10AA, 15A, and 15F; (173) 1T, 10AS, 10E, 10F, 10R, 10AA, 15A, and 15F; (174) 1T, 10AS, 10E, 10Q, 10Z, 10AA, 15A, and 15F; (175) 1T, 10AS, 10P, 10N, 10AA, 15A, and 15F; (176) 1T, 10AS, 10P, 10Y, 10Z, 10AA, 15A, and 15F; (177) 1T, 10AS, 10P, 10O, 15A, and 15F; (178) 1T, 10AS, 10E, 10F, 10R, 10AA, 15A, and 15F; (179) 10AT, 10E, 10F, 10G, 10S, 15A, and 15F; (180) 10AT, 10I, 10G, 10S, 15A, and 15F; (181) 10AT, 10K, 10S, 15A, and 15F; (182) 10AT, 10I, 10R, 10AA, 15A, and 15F; (183) 10AT, 10E, 10F, 10R, 10AA, 15A, and 15F; (184) 10AT, 10E, 10Q, 10Z, 10AA, 15A, and 15F; (185) 10AT, 10P, 10N, 10AA, 15A, and 15F; (186) 10AT, 10P, 10Y, 10Z, 10AA, 15A, and 15F; (187) 10AT, 10P, 10O, 15A, and 15F; (188) 10AT, 10E, 10F, 10R, 10AA, 15A, and 15F; (189) 14A, 14B, 14C, 14D, 14E, and 13A; (190) 16A, 16B, 16C, and 16D; (191) 17A, 17B, 17C, and 17D; (192) 17A, 17E, 17F, and 17D; (193) 17A, 17B, 17C, 17H, 17I, and 17J; (194) 18A, 18B, 18C, 18D, and 18E; and (195) 17A, 17E, 17F, 17H, 17I, and 17J, wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AE is a crotonyl-CoA:ACP transferase, wherein 10AS is an acetoacetyl-CoA synthase, wherein 10AT is an acetyl-CoA:acetyl-CoA acyltransferase, wherein 10AU is a 4-hydroxybutyryl-CoA dehydratase, wherein 13A is a 2-butanol desaturase, wherein 14A is an acetolactate synthase, wherein 14B is an acetolactate decarboxylase, wherein 14C is a butanediol dehydrogenase, wherein 14D is a butanediol dehydratase, wherein 14E is a butanol dehydrogenase, wherein 15A is a 1,3-butanediol kinase, wherein 15B is a 3-hydroxybutyrylphosphate kinase, 15C is a 3-hydroxybutyryldiphosphate lyase, wherein 15D is a 1,3-butanediol diphosphokinase, wherein 15E is a 1,3-butanediol dehydratase, wherein 15F is a 3-hydroxybutyrylphosphate lyase, wherein 16A is a 3-oxopent-4-enoyl-CoA thiolase, wherein 16B is a 3-oxopent-4-enoyl-CoA hydrolase, synthetase or transferase, wherein 16C is a 3-oxopent-4-enoate decarboxylase or spontaneous, wherein 16D is a 3-buten-2-one reductase, wherein 17A is a 3-oxo-4-hydroxypentanoyl-CoA thiolase, wherein 17B is a 3-oxo-4-hydroxypentanoyl-CoA transferase, synthetase or hydrolase, wherein 17C is a 3-oxo-4-hydroxypentanoate reductase, wherein 17D is a 3,4-dihydroxypentanoate decarboxylase, wherein 17E is a 3-oxo-4-hydroxypentanoyl-CoA reductase, wherein 17F is a 3,4-dihydroxypentanoyl-CoA transferase, synthetase or hydrolase, wherein 17H is a 3,4-dihydroxypentanoate dehydratase, wherein 17I is a 4-oxopentanoate reductase, wherein 17J is a 4-hyd4-oxoperoxypentanoate decarboxylase, wherein 18A is a 3-oxoadipyl-CoA thiolase, wherein 18B is a 3-oxoadipyl-CoA transferase, synthetase or hydrolase, wherein 18C is a 3-oxoadipate decarboxylase or spontaneous, wherein 18D is a 4-oxopentanoate reductase, wherein 18E is a 4-hydroxypentanoate decarboxylase.

In one aspect, the non-naturally occurring microbial organism a 3-buten-2-ol pathway described above further comprises a formaldehyde fixation pathway comprising at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase.

In one aspect, the non-naturally occurring microbial organism having a 3-buten-2-ol pathway described above further comprises a methanol metabolic pathway. In certain embodiments, the organism comprises at least one exogenous nucleic acid encoding a methanol metabolic pathway enzyme expressed in a sufficient amount to produce formaldehyde or produce or enhance the availability of reducing equivalents in the presence of methanol, wherein said methanol metabolic pathway comprises a pathway selected from: (1) 3J; (2) 3A and 3B; (3) 3A, 3B and 3C; (4) 3J, 3K and 3C; (5) 3J, 3M, and 3N; (6) 3J and 3L; (7) 3A, 3B, 3C, 3D, and 3E; (8) 3A, 3B, 3C, 3D, and 3F; (9) 3J, 3K, 3C, 3D, and 3E; (10) 3J, 3K, 3C, 3D, and 3F; (11) 3J, 3M, 3N, and 3O; (12) 3A, 3B, 3C, 3D, 3E, and 3G; (13) 3A, 3B, 3C, 3D, 3F, and 3G; (14) 3J, 3K, 3C, 3D, 3E, and 3G; (15) 3J, 3K, 3C, 3D, 3F, and 3G; (16) 3J, 3M, 3N, 3O, and 3G; (17) 3A, 3B, 3C, 3D, 3E, and 3I; (18) 3A, 3B, 3C, 3D, 3F, and 3I; (19) 3J, 3K, 3C, 3D, 3E, and 3I; (20) 3J, 3K, 3C, 3D, 3F, and 31; and (21) 3J, 3M, 3N, 3O, and 3I, wherein 3A is a methanol methyltransferase, wherein 3B is a methylenetetrahydrofolate reductase, wherein 3C is a methylenetetrahydrofolate dehydrogenase, wherein 3D is a methenyltetrahydrofolate cyclohydrolase, wherein 3E is a formyltetrahydrofolate deformylase, wherein 3F is a formyltetrahydrofolate synthetase, wherein 3G is a formate hydrogen lyase, wherein 3H is a hydrogenase, wherein 3I is a formate dehydrogenase, wherein 3J is a methanol dehydrogenase, wherein 3K is a formaldehyde activating enzyme or spontaneous, wherein 3L is a formaldehyde dehydrogenase, wherein 3M is a S-(hydroxymethyl)glutathione synthase or spontaneous, wherein 3N is a glutathione-dependent formaldehyde dehydrogenase, wherein 3O is a S-formylglutathione hydrolase,

In one aspect, the non-naturally occurring microbial organism having a 3-buten-2-ol pathway described above further comprises a methanol oxidation pathway. In certain embodiments, the organism comprises at least one exogenous nucleic acid encoding a methanol oxidation pathway enzyme expressed in a sufficient amount to produce formaldehyde in the presence of methanol, wherein said methanol oxidation pathway comprises 1A, wherein 1A a methanol dehydrogenase.

In one aspect, the non-naturally occurring microbial organism having a 3-buten-2-ol pathway described above further comprises 3H or 3P, wherein 3H is a hydrogenase, wherein 3P a carbon monoxide dehydrogenase. In certain embodiments, the organism comprises an exogenous nucleic acid encoding said hydrogenase or said carbon monoxide dehydrogenase.

In certain embodiments, provided herein is a non-naturally occurring microbial organism having a formaldehyde fixation pathway, a formate assimilation pathway, a methanol metabolic pathway, a methanol oxidation pathway, a hydrogenase, a carbon monoxide dehydrogenase or any combination described above, wherein the organism further comprises a 3-buten-2-ol pathway. In certain embodiments, the microbial organism comprises at least one exogenous nucleic acid encoding a 3-buten-2-ol pathway enzyme expressed in a sufficient amount to produce 3-buten-2-ol, wherein said 3-buten-2-ol pathway comprises a pathway as shown in FIGS. 1, 10 and 13-18 selected from: (1) 10A, 10D, 10E, 10F, 10G, 10S, 15A, 15B, and 15C; (2) 10A, 10D, 10I, 10G, 10S, 15A, 15B, and 15C; (3) 10A, 10D, 10K, 10S, 15A, 15B, and 15C; (4) 10A, 10H, 10F, 10G, 10S, 15A, 15B, and 15C; (5) 10A, 10J, 10G, 10S, 15A, 15B, and 15C; (6) 10A, 10J, 10R, 10AA, 15A, 15B, and 15C; (7) 10A, 10H, 10F, 10R, 10AA, 15A, 15B, and 15C; (8) 10A, 10H, 10Q, 10Z, 10AA, 15A, 15B, and 15C; (9) 10A, 10D, 10I, 10R, 10AA, 15A, 15B, and 15C; (10) 10A, 10D, 10E, 10F, 10R, 10AA, 15A, 15B, and 15C; (11) 10A, 10D, 10E, 10Q, 10Z, 10AA, 15A, 15B, and 15C; (12) 10A, 10D, 10P, 10N, 10AA, 15A, 15B, and 15C; (13) 10A, 10D, 10P, 10Y, 10Z, 10AA, 15A, 15B, and 15C; (14) 10A, 10B, 10M, 10AA, 15A, 15B, and 15C; (15) 10A, 10B, 10L, 10Z, 10AA, 15A, 15B, and 15C; (16) 10A, 10B, 10X, 10N, 10AA, 15A, 15B, and 15C; (17) 10A, 10B, 10X, 10Y, 10Z, 10AA, 15A, 15B, and 15C; (18) 10A, 10D, 10P, 10O, 15A, 15B, and 15C; (19) 10A, 10B, 10X, 10O, 15A, 15B, and 15C; (20) 10A, 10D, 10E, 10F, 10R, 10AA, 15A, 15B, and 15C; (21) 10A, 10D, 10E, 10F, 10G, 10S, 15A, 15B, and 15C; (22) 10A, 10B, 10C, 10AE, 10AB, 10Y, 10Z, 10AA, 15A, 15B, and 15C; (23) 10A, 10B, 10C, 10AE, 10AB, 10N, 10AA, 15A, 15B, and 15C; (24) 10A, 10B, 10C, 10AE, 10AB, 10O, 15A, 15B, and 15C; (25) 10AU, 10AB, 10Y, 10Z, 10AA, 15A, 15B, and 15C; (26) 10AU, 10AB, 10N, 10AA, 15A, 15B, and 15C; (27) 10AU, 10AB, 10O, 15A, 15B, and 15C; (28) 1T, 10AS, 10E, 10F, 10G, 10S, 15A, 15B, and 15C; (29) 1T, 10AS, 10I, 10G, 10S, 15A, 15B, and 15C; (30) 1T, 10AS, 10K, 10S, 15A, 15B, and 15C; (31) 1T, 10AS, 10I, 10R, 10AA, 15A, 15B, and 15C; (32) 1T, 10AS, 10E, 10F, 10R, 10AA, 15A, 15B, and 15C; (33) 1T, 10AS, 10E, 10Q, 10Z, 10AA, 15A, 15B, and 15C; (34) 1T, 10AS, 10P, 10N, 10AA, 15A, 15B, and 15C; (35) 1T, 10AS, 10P, 10Y, 10Z, 10AA, 15A, 15B, and 15C; (36) 1T, 10AS, 10P, 10O, 15A, 15B, and 15C; (37) 1T, 10AS, 10E, 10F, 10R, 10AA, 15A, 15B, and 15C; (38) 10AT, 10E, 10F, 10G, 10S, 15A, 15B, and 15C; (39) 10AT, 10I, 10G, 10S, 15A, 15B, and 15C; (40) 10AT, 10K, 10S, 15A, 15B, and 15C; (41) 10AT, 10I, 10R, 10AA, 15A, 15B, and 15C; (42) 10AT, 10E, 10F, 10R, 10AA, 15A, 15B, and 15C; (43) 10AT, 10E, 10Q, 10Z, 10AA, 15A, 15B, and 15C; (44) 10AT, 10P, 10N, 10AA, 15A, 15B, and 15C; (45) 10AT, 10P, 10Y, 10Z, 10AA, 15A, 15B, and 15C; (46) 10AT, 10P, 10O, 15A, 15B, and 15C; (47) 10AT, 10E, 10F, 10R, 10AA, 15A, 15B, and 15C; (48) 10A, 10D, 10E, 10F, 10G, 10S, and 15D; (49) 10A, 10D, 10I, 10G, 10S, and 15D; (50) 10A, 10D, 10K, 10S, and 15D; (51) 10A, 10H, 10F, 10G, 10S, and 15D; (52) 10A, 10J, 10G, 10S, and 15D; (53) 10A, 10J, 10R, 10AA, and 15D; (54) 10A, 10H, 10F, 10R, 10AA, and 15D; (55) 10A, 10H, 10Q, 10Z, 10AA, and 15D; (56) 10A, 10D, 10I, 10R, 10AA, and 15D; (57) 10A, 10D, 10E, 10F, 10R, 10AA, and 15D; (58) 10A, 10D, 10E, 10Q, 10Z, 10AA, and 15D; (59) 10A, 10D, 10P, 10N, 10AA, and 15D; (60) 10A, 10D, 10P, 10Y, 10Z, 10AA, and 15D; (61) 10A, 10B, 10M, 10AA, and 15D; (62) 10A, 10B, 10L, 10Z, 10AA, and 15D; (63) 10A, 10B, 10X, 10N, 10AA, and 15D; (64) 10A, 10B, 10X, 10Y, 10Z, 10AA, and 15D; (65) 10A, 10D, 10P, 10O, and 15D; (66) 10A, 10B, 10X, 10O, and 15D; (67) 10A, 10D, 10E, 10F, 10R, 10AA, and 15D; (68) 10A, 10D, 10E, 10F, 10G, 10S, and 15D; (69) 10A, 10B, 10C, 10AE, 10AB, 10Y, 10Z, 10AA, and 15D; (70) 10A, 10B, 10C, 10AE, 10AB, 10N, 10AA, and 15D; (71) 10A, 10B, 10C, 10AE, 10AB, 10O, and 15D; (72) 10AU, 10AB, 10Y, 10Z, 10AA, and 15D; (73) 10AU, 10AB, 10N, 10AA, and 15D; (74) 10AU, 10AB, 10O, and 15D; (75) 1T, 10AS, 10E, 10F, 10G, 10S, and 15D; (76) 1T, 10AS, 10I, 10G, 10S, and 15D; (77) 1T, 10AS, 10K, 10S, and 15D; (78) 1T, 10AS, 10I, 10R, 10AA, and 15D; (79) 1T, 10AS, 10E, 10F, 10R, 10AA, and 15D; (80) 1T, 10AS, 10E, 10Q, 10Z, 10AA, and 15D; (81) 1T, 10AS, 10P, 10N, 10AA, and 15D; (82) 1T, 10AS, 10P, 10Y, 10Z, 10AA, and 15D; (83) 1T, 10AS, 10P, 10O, and 15D; (84) 1T, 10AS, 10E, 10F, 10R, 10AA, and 15D; (85) 10AT, 10E, 10F, 10G, 10S, and 15D; (86) 10AT, 10I, 10G, 10S, and 15D; (87) 10AT, 10K, 10S, and 15D; (88) 10AT, 10I, 10R, 10AA, and 15D; (89) 10AT, 10E, 10F, 10R, 10AA, and 15D; (90) 10AT, 10E, 10Q, 10Z, 10AA, and 15D; (91) 10AT, 10P, 10N, 10AA, and 15D; (92) 10AT, 10P, 10Y, 10Z, 10AA, and 15D; (93) 10AT, 10P, 10O, and 15D; (94) 10AT, 10E, 10F, 10R, 10AA, and 15D; (95) 10A, 10D, 10E, 10F, 10G, 10S, 15E, and 15C; (96) 10A, 10D, 10I, 10G, 10S, 15E, and 15C; (97) 10A, 10D, 10K, 10S, 15E, and 15C; (98) 10A, 10H, 10F, 10G, 10S, 15E, and 15C; (99) 10A, 10J, 10G, 10S, 15E, and 15C; (100) 10A, 10J, 10R, 10AA, 15E, and 15C; (101) 10A, 10H, 10F, 10R, 10AA, 15E, and 15C; (102) 10A, 10H, 10Q, 10Z, 10AA, 15E, and 15C; (103) 10A, 10D, 10I, 10R, 10AA, 15E, and 15C; (104) 10A, 10D, 10E, 10F, 10R, 10AA, 15E, and 15C; (105) 10A, 10D, 10E, 10Q, 10Z, 10AA, 15E, and 15C; (106) 10A, 10D, 10P, 10N, 10AA, 15E, and 15C; (107) 10A, 10D, 10P, 10Y, 10Z, 10AA, 15E, and 15C; (108) 10A, 10B, 10M, 10AA, 15E, and 15C; (109) 10A, 10B, 10L, 10Z, 10AA, 15E, and 15C; (110) 10A, 10B, 10X, 10N, 10AA, 15E, and 15C; (111) 10A, 10B, 10X, 10Y, 10Z, 10AA, 15E, and 15C; (112) 10A, 10D, 10P, 10O, 15E, and 15C; (113) 10A, 10B, 10X, 10O, 15E, and 15C; (114) 10A, 10D, 10E, 10F, 10R, 10AA, 15E, and 15C; (115) 10A, 10D, 10E, 10F, 10G, 10S, 15E, and 15C; (116) 10A, 10B, 10C, 10AE, 10AB, 10Y, 10Z, 10AA, 15E, and 15C; (117) 10A, 10B, 10C, 10AE, 10AB, 10N, 10AA, 15E, and 15C; (118) 10A, 10B, 10C, 10AE, 10AB, 10O, 15E, and 15C; (119) 10AU, 10AB, 10Y, 10Z, 10AA, 15E, and 15C; (120) 10AU, 10AB, 10N, 10AA, 15E, and 15C; (121) 10AU, 10AB, 10O, 15E, and 15C; (122) 1T, 10AS, 10E, 10F, 10G, 10S, 15E, and 15C; (123) 1T, 10AS, 10I, 10G, 10S, 15E, and 15C; (124) 1T, 10AS, 10K, 10S, 15E, and 15C; (125) 1T, 10AS, 10I, 10R, 10AA, 15E, and 15C; (126) 1T, 10AS, 10E, 10F, 10R, 10AA, 15E, and 15C; (127) 1T, 10AS, 10E, 10Q, 10Z, 10AA, 15E, and 15C; (128) 1T, 10AS, 10P, 10N, 10AA, 15E, and 15C; (129) 1T, 10AS, 10P, 10Y, 10Z, 10AA, 15E, and 15C; (130) 1T, 10AS, 10P, 10O, 15E, and 15C; (131) 1T, 10AS, 10E, 10F, 10R, 10AA, 15E, and 15C; (132) 10AT, 10E, 10F, 10G, 10S, 15E, and 15C; (133) 10AT, 10I, 10G, 10S, 15E, and 15C; (134) 10AT, 10K, 10S, 15E, and 15C; (135) 10AT, 10I, 10R, 10AA, 15E, and 15C; (136) 10AT, 10E, 10F, 10R, 10AA, 15E, and 15C; (137) 10AT, 10E, 10Q, 10Z, 10AA, 15E, and 15C; (138) 10AT, 10P, 10N, 10AA, 15E, and 15C; (139) 10AT, 10P, 10Y, 10Z, 10AA, 15E, and 15C; (140) 10AT, 10P, 10O, 15E, and 15C; (141) 10AT, 10E, 10F, 10R, 10AA, 15E, and 15C; (142) 10A, 10D, 10E, 10F, 10G, 10S, 15A, and 15F; (143) 10A, 10D, 10I, 10G, 10S, 15A, and 15F; (144) 10A, 10D, 10K, 10S, 15A, and 15F; (145) 10A, 10H, 10F, 10G, 10S, 15A, and 15F; (146) 10A, 10J, 10G, 10S, 15A, and 15F; (147) 10A, 10J, 10R, 10AA, 15A, and 15F; (148) 10A, 10H, 10F, 10R, 10AA, 15A, and 15F; (149) 10A, 10H, 10Q, 10Z, 10AA, 15A, and 15F; (150) 10A, 10D, 10I, 10R, 10AA, 15A, and 15F; (151) 10A, 10D, 10E, 10F, 10R, 10AA, 15A, and 15F; (152) 10A, 10D, 10E, 10Q, 10Z, 10AA, 15A, and 15F; (153) 10A, 10D, 10P, 10N, 10AA, 15A, and 15F; (154) 10A, 10D, 10P, 10Y, 10Z, 10AA, 15A, and 15F; (155) 10A, 10B, 10M, 10AA, 15A, and 15F; (156) 10A, 10B, 10L, 10Z, 10AA, 15A, and 15F; (157) 10A, 10B, 10X, 10N, 10AA, 15A, and 15F; (158) 10A, 10B, 10X, 10Y, 10Z, 10AA, 15A, and 15F; (159) 10A, 10D, 10P, 10O, 15A, and 15F; (160) 10A, 10B, 10X, 10O, 15A, and 15F; (161) 10A, 10D, 10E, 10F, 10R, 10AA, 15A, and 15F; (162) 10A, 10D, 10E, 10F, 10G, 10S, 15A, and 15F; (163) 10A, 10B, 10C, 10AE, 10AB, 10Y, 10Z, 10AA, 15A, and 15F; (164) 10A, 10B, 10C, 10AE, 10AB, 10N, 10AA, 15A, and 15F; (165) 10A, 10B, 10C, 10AE, 10AB, 10O, 15A, and 15F; (166) 10AU, 10AB, 10Y, 10Z, 10AA, 15A, and 15F; (167) 10AU, 10AB, 10N, 10AA, 15A, and 15F; (168) 10AU, 10AB, 10O, 15A, and 15F; (169) 1T, 10AS, 10E, 10F, 10G, 10S, 15A, and 15F; (170) 1T, 10AS, 10I, 10G, 10S, 15A, and 15F; (171) 1T, 10AS, 10K, 10S, 15A, and 15F; (172) 1T, 10AS, 10I, 10R, 10AA, 15A, and 15F; (173) 1T, 10AS, 10E, 10F, 10R, 10AA, 15A, and 15F; (174) 1T, 10AS, 10E, 10Q, 10Z, 10AA, 15A, and 15F; (175) 1T, 10AS, 10P, 10N, 10AA, 15A, and 15F; (176) 1T, 10AS, 10P, 10Y, 10Z, 10AA, 15A, and 15F; (177) 1T, 10AS, 10P, 10O, 15A, and 15F; (178) 1T, 10AS, 10E, 10F, 10R, 10AA, 15A, and 15F; (179) 10AT, 10E, 10F, 10G, 10S, 15A, and 15F; (180) 10AT, 10I, 10G, 10S, 15A, and 15F; (181) 10AT, 10K, 10S, 15A, and 15F; (182) 10AT, 10I, 10R, 10AA, 15A, and 15F; (183) 10AT, 10E, 10F, 10R, 10AA, 15A, and 15F; (184) 10AT, 10E, 10Q, 10Z, 10AA, 15A, and 15F; (185) 10AT, 10P, 10N, 10AA, 15A, and 15F; (186) 10AT, 10P, 10Y, 10Z, 10AA, 15A, and 15F; (187) 10AT, 10P, 10O, 15A, and 15F; (188) 10AT, 10E, 10F, 10R, 10AA, 15A, and 15F; (189) 14A, 14B, 14C, 14D, 14E, and 13A; (190) 16A, 16B, 16C, and 16D; (191) 17A, 17B, 17C, and 17D; (192) 17A, 17E, 17F, and 17D; (193) 17A, 17B, 17C, 17H, 17I, and 17J; (194) 18A, 18B, 18C, 18D, and 18E; (195) 13A; and (196) 17A, 17E, 17F, 17H, 17I, and 17J, wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AE is a crotonyl-CoA:ACP transferase, wherein 10AS is an acetoacetyl-CoA synthase, wherein 10AT is an acetyl-CoA:acetyl-CoA acyltransferase, wherein 10AU is a 4-hydroxybutyryl-CoA dehydratase, wherein 13A is a 2-butanol desaturase, wherein 14A is an acetolactate synthase, wherein 14B is an acetolactate decarboxylase, wherein 14C is a butanediol dehydrogenase, wherein 14D is a butanediol dehydratase, wherein 14E is a butanol dehydrogenase, wherein 15A is a 1,3-butanediol kinase, wherein 15B is a 3-hydroxybutyrylphosphate kinase, 15C is a 3-hydroxybutyryldiphosphate lyase, wherein 15D is a 1,3-butanediol diphosphokinase, wherein 15E is a 1,3-butanediol dehydratase, wherein 15F is a 3-hydroxybutyrylphosphate lyase, wherein 16A is a 3-oxopent-4-enoyl-CoA thiolase, wherein 16B is a 3-oxopent-4-enoyl-CoA hydrolase, synthetase or transferase, wherein 16C is a 3-oxopent-4-enoate decarboxylase or spontaneous, wherein 16D is a 3-buten-2-one reductase, wherein 17A is a 3-oxo-4-hydroxypentanoyl-CoA thiolase, wherein 17B is a 3-oxo-4-hydroxypentanoyl-CoA transferase, synthetase or hydrolase, wherein 17C is a 3-oxo-4-hydroxypentanoate reductase, wherein 17D is a 3,4-dihydroxypentanoate decarboxylase, wherein 17E is a 3-oxo-4-hydroxypentanoyl-CoA reductase, wherein 17F is a 3,4-dihydroxypentanoyl-CoA transferase, synthetase or hydrolase, wherein 17H is a 3,4-dihydroxypentanoate dehydratase, wherein 17I is a 4-oxopentanoate reductase, wherein 17J is a 4-hyd4-oxoperoxypentanoate decarboxylase, wherein 18A is a 3-oxoadipyl-CoA thiolase, wherein 18B is a 3-oxoadipyl-CoA transferase, synthetase or hydrolase, wherein 18C is a 3-oxoadipate decarboxylase or spontaneous, wherein 18D is a 4-oxopentanoate reductase, wherein 18E is a 4-hydroxypentanoate decarboxylase.

In certain embodiments, provided herein is a non-naturally occurring microbial organism having a formaldehyde fixation pathway, a formate assimilation pathway, a methanol oxidation pathway, and a butadiene, crotyl alcohol, 1,3-butanediol, or 3-buten-2-ol pathway. In some aspects, the organism comprises at least one exogenous nucleic acid encoding a formaldehyde fixation pathway enzyme expressed in a sufficient amount to produce pyruvate, wherein said formaldehyde fixation pathway comprises: (1) 1B and 1C; or (2) 1D, wherein 1B is a 3-hexulose-6-phosphate synthase, wherein 1C is a 6-phospho-3-hexuloisomerase, wherein 1D is a dihydroxyacetone synthase, comprises at least one exogenous nucleic acid encoding a formate assimilation pathway enzyme expressed in a sufficient amount to produce formaldehyde, pyruvate, or acetyl-CoA, wherein said formate assimilation pathway comprises a pathway selected from: (3) 1E; (4) 1F, and 1G; (5) 1H, 1I, 1J, and 1K; (6) 1H, 1I, 1J, 1L, 1M, and 1N; (7) 1E, 1H, 1I, 1J, 1L, 1M, and 1N; (8) 1F, 1G, 1H, 1I, 1J, 1L, 1M, and 1N; (9) 1K, 1H, 1I, 1J, 1L, 1M, and 1N; and (10) 1H, 1I, 1J, 1O, and 1P5, comprises at least one exogenous nucleic acid encoding a methanol oxidation pathway enzyme expressed in a sufficient amount to produce formaldehyde in the presence of methanol, wherein said methanol oxidation pathway comprises a methanol dehydrogenase, and comprises at least one exogenous nucleic acid encoding a butadiene, crotyl alcohol, 1,3-butanediol, or 3-buten-2-ol pathway enzyme expressed in a sufficient amount to produce butadiene, crotyl alcohol, 1,3-butanediol, or 3-buten-2-ol, wherein said butadiene, crotyl alcohol, 1,3-butanediol, or 3-buten-2-ol pathway comprises a pathway selected from: steps 1T, 10AS, 10P, 10N, 10AA, 15A, 15B, 15C, and 15G; or steps 10AT, 10P, 10N, 10AA, 15A, 15B, 15C, and 15G; or steps 14A, 14B, 14C, 14D, 14E, 13A, and 13B; or steps 17A, 17B, 17C, 17D, and 17G; or steps 17A, 17E, 17F, 17D, and 17G; or steps 18A, 18B, 18C, 18D, 18E, and 18F; or steps 1T, 10AS, 10P, 10AB, 10V, 10AH, 11A, 11B, and 11C; or steps 10AT, 10P, 10AB, 10V, 10AH, 11A, 11B, and 11C; or steps 13A and 13B; or steps 1T, 10AS, 10P, 10AB, 10V, and 10AH; 10AS, 10P, 10AB, 10AF, 10AG, and 10AH; or steps 1T, 10AS, 10P, 10AB, and 10W; or steps 10AT, 10P, 10AB, 10V, and 10AH; or steps 10AT, 10P, 10AB, 10AF, 10AG, and 10AH; or steps 10AT, 10P, 10AB, and 10W; or steps 1T, 10AS, 10P, 10N, and 10AA; or steps 1T, 10AS, 10P, 10Y, 10Z, and 10AA; or steps 10AT, 10P, 10N, and 10AA; or steps 10AT, 10P, 10Y, 10Z, and 10AA; or steps 10AS, 10P, 10N, 10AA, 15A, 15B, and 15C; or steps 10AT, 10P, 10N, 10AA, 15A, 15B; or steps 14A, 14B, 14C, 14D, 14E, and 13A; or steps 17A, 17B, 17C, and 17D; or steps 17A, 17E, 17F, and 17D; or steps 18A, 18B, 18C, 18D, and 18E. In certain embodiments, said formaldehyde fixation pathway comprises: (1) 1B and 1C. In certain embodiments, said formaldehyde fixation pathway comprises: (2) 1D. In certain embodiments, said formate assimilation pathway comprises: (3) 1E. In certain embodiments, said formate assimilation pathway comprises: (4) 1F, and 1G. In certain embodiments, said formate assimilation pathway comprises: (5) 1H, 1I, 1J, and 1K. In certain embodiments, said formate assimilation pathway comprises: (6) 1H, 1I, 1J, 1L, 1M, and 1N. In certain embodiments, said formate assimilation pathway comprises: (7) 1E, 1H, 1I, 1J, 1L, 1M, and 1N. In certain embodiments, said formate assimilation pathway comprises: (8) 1F, 1G, 1H, 1I, 1J, 1L, 1M, and 1N. In certain embodiments, said formate assimilation pathway comprises: (9) 1K, 1H, 1I, 1J, 1L, 1M, and 1N. In certain embodiments, said formate assimilation pathway comprises: (10) 1H, 1I, 1J, 1O, and 1P5. In certain embodiments, said butadiene, crotyl alcohol, 1,3-butanediol, or 3-buten-2-ol pathway comprises: 1T, 10AS, 10P, 10N, 10AA, 15A, 15B, 15C, and 15G. In certain embodiments, said butadiene, crotyl alcohol, 1,3-butanediol, or 3-buten-2-ol pathway comprises: 10AT, 10P, 10N, 10AA, 15A, 15B, 15C, and 15G. In certain embodiments, said butadiene, crotyl alcohol, 1,3-butanediol, or 3-buten-2-ol pathway comprises: 14A, 14B, 14C, 14D, 14E, 13A, and 13B. In certain embodiments, said butadiene, crotyl alcohol, 1,3-butanediol, or 3-buten-2-ol pathway comprises: 17A, 17B, 17C, 17D, and 17G. In certain embodiments, said butadiene, crotyl alcohol, 1,3-butanediol, or 3-buten-2-ol pathway comprises: 17A, 17E, 17F, 17D, and 17G. In certain embodiments, said butadiene, crotyl alcohol, 1,3-butanediol, or 3-buten-2-ol pathway comprises: 18A, 18B, 18C, 18D, 18E, and 18F. In certain embodiments, said butadiene, crotyl alcohol, 1,3-butanediol, or 3-buten-2-ol pathway comprises: 1T, 10AS, 10P, 10AB, 10V, 10AH, 11A, 11B, and 11C. In certain embodiments, said butadiene, crotyl alcohol, 1,3-butanediol, or 3-buten-2-ol pathway comprises: 10AT, 10P, 10AB, 10V, 10AH, 11A, 11B, and 11C. In certain embodiments, said butadiene, crotyl alcohol, 1,3-butanediol, or 3-buten-2-ol pathway comprises: 13A and 13B; or steps 1T, 10AS, 10P, 10AB, 10V, and 10AH. In certain embodiments, said butadiene, crotyl alcohol, 1,3-butanediol, or 3-buten-2-ol pathway comprises: 10AS, 10P, 10AB, 10AF, 10AG, and 10AH. In certain embodiments, said butadiene, crotyl alcohol, 1,3-butanediol, or 3-buten-2-ol pathway comprises: 1T, 10AS, 10P, 10AB, and 10W. In certain embodiments, said butadiene, crotyl alcohol, 1,3-butanediol, or 3-buten-2-ol pathway comprises: 10AT, 10P, 10AB, 10V, and 10AH. In certain embodiments, said butadiene, crotyl alcohol, 1,3-butanediol, or 3-buten-2-ol pathway comprises: 10AT, 10P, 10AB, 10AF, 10AG, and 10AH. In certain embodiments, said butadiene, crotyl alcohol, 1,3-butanediol, or 3-buten-2-ol pathway comprises: 10AT, 10P, 10AB, and 10W. In certain embodiments, said butadiene, crotyl alcohol, 1,3-butanediol, or 3-buten-2-ol pathway comprises: 1T, 10AS, 10P, 10N, and 10AA. In certain embodiments, said butadiene, crotyl alcohol, 1,3-butanediol, or 3-buten-2-ol pathway comprises: 1T, 10AS, 10P, 10Y, 10Z, and 10AA. In certain embodiments, said butadiene, crotyl alcohol, 1,3-butanediol, or 3-buten-2-ol pathway comprises: 10AT, 10P, 10N, and 10AA. In certain embodiments, said butadiene, crotyl alcohol, 1,3-butanediol, or 3-buten-2-ol pathway comprises: 10AT, 10P, 10Y, 10Z, and 10AA. In certain embodiments, said butadiene, crotyl alcohol, 1,3-butanediol, or 3-buten-2-ol pathway comprises: 10AS, 10P, 10N, 10AA, 15A, 15B, and 15C. In certain embodiments, said butadiene, crotyl alcohol, 1,3-butanediol, or 3-buten-2-ol pathway comprises: 10AT, 10P, 10N, 10AA, 15A, 15B. In certain embodiments, said butadiene, crotyl alcohol, 1,3-butanediol, or 3-buten-2-ol pathway comprises: 14A, 14B, 14C, 14D, 14E, and 13A. In certain embodiments, said butadiene, crotyl alcohol, 1,3-butanediol, or 3-buten-2-ol pathway comprises: 17A, 17B, 17C, and 17D. In certain embodiments, said butadiene, crotyl alcohol, 1,3-butanediol, or 3-buten- 2-ol pathway comprises: 17A, 17E, 17F, and 17D; or steps 18A, 18B, 18C, 18D, and 18E.

In an additional embodiment, the invention provides a non-naturally occurring microbial organism having a butadiene, 1,3-butanediol, crotyl alcohol or 3-buten-2-ol pathway, wherein the non-naturally occurring microbial organism comprises at least one exogenous nucleic acid encoding an enzyme or protein that converts a substrate to a product selected from the group consisting of MeOH to Fald, Fald to H6P, Fald to DHA and G3P, PYR to formate and ACCOA, PYR to CO2 and ACCOA, CO2 to formate, formate to Fald, formate to Formyl-CoA, Formyl-CoA to Fald, Formate to FTHF, FTHF to methenyl-THF, methenyl-THF to methylene-THF, methylene-THF to Fald, methylene-THF to glycine, glycine to serine, serine to PYR, methylene-THF to methyl-THF, methyl-THF to ACCOA, ACCOA to MALCOA, methanol to methyl-THF, methyl-THF to methylene-THF, formaldehyde to methylene-THF, methylene-THF to methenyl-THF, formyl-THF to formate, formate to CO2, formaldehyde to S-hydroxymethylglutathione, S-hydroxymethylglutathione to S-formylglutathione to formate, formaldehyde to formate, malonyl-ACP and acetyl-CoA or acetyl-ACP to acetoacetyl-ACP, acetoacetyl-ACP to 3-hydroxybutyryl-ACP, 3-hydroxybutyryl-ACP to crotonyl-ACP, acetoacetyl-ACP to acetoacetyl-CoA, malonyl-CoA and acetyl-CoA to acetoacetyl-CoA, acetoacetyl-CoA to acetoacetate, acetoacetate to 3-oxobutyraldehyde, 3-oxobutyraldehyde to 4-hydroxy-2-butanone, acetoacetyl-ACP to acetoacetate, acetoacetyl-CoA to 3-oxobutyraldehyde, acetoacetyl-ACP to 3-oxobutyraldehyde, acetoacetyl-CoA to 4-hydroxy-2-butanone, 3-hydroxybutyryl-ACP to 3-hydroxybutyrate, 3-hydroxybutyryl-ACP to 3-hydroxybutyraldehyde, 3-hydroxybutyryl-CoA to 3-hydroxybutyraldehyde, 3-hydroxybutyryl-CoA to 1,3-butanediol, acetoacetyl-CoA to 3-hydroxybutyryl-CoA, acetoacetate to 3-hydroxybutyrate, 3-oxobutyraldehyde to 3-hydroxybutyraldehyde, 4-hydroxy-2-butanone to 1,3-butanediol, crotonyl-ACP to crotonate, crotonyl-ACP to crotonaldehyde, crotonyl-CoA to crotonaldehyde, crotonyl-CoA to crotyl alcohol, 3-hydroxybutyryl-ACP to 3-hydroxybutyryl-CoA, 3-hydroxybutyryl-CoA to 3-hydroxybutyrate, 3-hydroxybutyrate to 3-hydroxybutyraldehyde, 3-hydroxybutyraldehyde to 1,3-butanediol, 3-hydroxybutyryl-CoA to crotonyl-CoA, 3-hydroxybutyrate to crotonate, 3-hydroxybutyraldehyde to crotonaldehyde, crotonyl-ACP to crotonyl-CoA, crotonyl-CoA to crotonate, crotonate to crotonaldehyde, crotonaldehyde to crotyl alcohol, crotyl alcohol to 2-butenyl-4-phosphate, 2-butenyl-4-phosphate to 2-butenyl-4-diphosphate, crotyl alcohol to 2-butenyl-4-diphosphate, 2-butenyl-4-diphosphate to butadiene, crotyl alcohol to butadiene, malonyl-CoA and acetyl-CoA to 3-oxoglutaryl-CoA, 3-oxoglutaryl-CoA to 3-hydroxyglutaryl-CoA to 3-hydroxy-5-oxopentanoate, 3-hydroxy-5-oxopentanoate to 3,5-dihydroxy pentanoate, 3,5-dihydroxy pentanoate to 3-hydroxy-5-phosphonatooxypentanoate, 3-hydroxy-5-phosphonatooxypentanoate to 3-hydroxy-5-[hydroxy(phosphonooxy)phosphoryl]oxy pentanoate, 3-hydroxy- 5-[hydroxy(phosphonooxy)phosphoryl]oxy pentanoate to butenyl 4-biphosphate, butenyl 4-biphosphate to 2-butenyl 4-diphosphate, 2-butenyl 4-diphosphate to butadiene, 2-butanol to 3-buten-2-ol, 3-buten-2-ol to butadiene, pyruvate to acetolactate, acetolactate to acetoin, acetoin to 2,3-butanediol, 2,3-butanediol to 2-butanal, 2-butanal to 2-butanol, 1,3-butanediol to 3-hydroxybutyryl phosphate, 3-hydroxybutyryl phosphate to 3-hydroxybutyryl diphosphate, 3-hydroxybutyryl diphosphate to 3-buten-2-ol, 1,3-butanediol to 3-hydroxybutyryl diphosphate, 1,3-butanediol to 3-buten- 2-ol, acrylyl-CoA and acetyl-CoA to 3-oxopent-4-enoyl-CoA, 3-oxopent-4-enoyl-CoA to 3-oxopent-4-enoate, 3-oxopent-4-enoate to 3-buten-2-one, 3-buten-2-one to 3-buten-2-ol, lactoyl-CoA and acetyl-CoA to 3-oxo-4-hydroxy pentanoyl-CoA, 3-oxo-4-hydroxy pentanoyl-CoA to 3-oxo-4-hydroxy pentanoate, 3-oxo-4-hydroxy pentanoate to 3,4-dihydroxypentanoate, 3,4-dihydroxypentanoate to 3-buten-2-ol, 3-oxo-4hydroxy pentanoyl-CoA to 3,4-dihydroxypentanoyl-CoA, 3,4-dihydroxypentanoyl-CoA to 3,4-dihydroxypentanoate, 3,4-dihydroxypentanoate to 4-oxopentanoate, 4-oxopentanoate to 4-hydroxypentanoate, 4-hydroxypentanoate to 3-buten-2-ol, succinyl-CoA and acetyl-CoA to 3-oxoadipyl-CoA, 3-oxoadipyl-CoA to 3-oxoadipate, 3-oxoadipate to 4-oxopentanoate, 4-oxopentanoate to 4-hydroxypentanoate, 4-hydroxypentanoate to 3-butene-2-ol. One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, the invention provides a non-naturally occurring microbial organism containing at least one exogenous nucleic acid encoding an enzyme or protein, where the enzyme or protein converts the substrates and products of a butadiene, 1,3-butanediol, crotyl alcohol or 3-buten-2-ol pathway, such as that shown in FIGS. 1-18.

While generally described herein as a microbial organism that contains a butadiene, 1,3-butanediol, crotyl alcohol or 3-buten-2-ol pathway, it is understood that the invention additionally provides a non-naturally occurring microbial organism comprising at least one exogenous nucleic acid encoding a butadiene, 1,3-butanediol, crotyl alcohol or 3-buten-2-ol pathway enzyme expressed in a sufficient amount to produce an intermediate of a butadiene, 1,3-butanediol, crotyl alcohol or 3-buten-2-ol pathway. For example, as disclosed herein, a butadiene, 1,3-butanediol, crotyl alcohol or 3-buten-2-ol pathway is exemplified in FIG. 1-18. Therefore, in addition to a microbial organism containing a butadiene, 1,3-butanediol, crotyl alcohol or 3-buten-2-ol pathway that produces butadiene, 1,3-butanediol, crotyl alcohol or 3-buten-2-ol, the invention additionally provides a non-naturally occurring microbial organism comprising at least one exogenous nucleic acid encoding a butadiene, 1,3-butanediol, crotyl alcohol or 3-buten-2-ol pathway enzyme, where the microbial organism produces a butadiene, 1,3-butanediol, crotyl alcohol or 3-buten-2-ol pathway intermediate, for example, acetoacetyl-CoA, acetoacetate, 3-oxobutyraldehyde, acetoacetyl-ACP, acetoacetyl-CoA, acetoacetyl-ACP, acetoacetyl-CoA, 3-hydroxybutyryl-ACP, 3-hydroxybutyryl-ACP, 3-hydroxybutyryl-CoA, 3-hydroxybutyryl-CoA, acetoacetyl-CoA, acetoacetate, 3-oxobutyraldehyde, 4-hydroxy-2-butanone, crotonyl-ACP, crotonyl-CoA, 3-hydroxybutyryl-ACP, 3-hydroxybutyryl-CoA, 3-hydroxybutyrate, 3-hydroxybutyraldehyde, crotonaldehyde, crotonyl-ACP, crotonyl-CoA, crotonate, crotonaldehyde, 2-butenyl-4-phosphate, 2-butenyl-4-diphosphate, 3-oxoglutaryl-CoA, 3-hydroxy-5-oxopentanoate, 3,5-dihydroxy pentanoate, 3-hydroxy-5-phosphonatooxypentanoate, 3-hydroxy-5-[hydroxy(phosphonooxy)phosphoryl]oxy pentanoate, butenyl 4-biphosphate, 2-butenyl 4-diphosphate, 2-butanol, acetolactate, acetoin, 2,3-butanediol, 3-hydroxybutyryl phosphate, 3-hydroxybutyryl diphosphate, 3-oxopent-4-enoyl-CoA, 3-oxopent-4-enoate, 3-buten-2-one, 3-oxo-4-hydroxy pentanoyl-CoA, 3-oxo-4-hydroxy pentanoate, 3,4-dihydroxypentanoate, 3,4-dihydroxypentanoyl-CoA, 3,4-dihydroxypentanoate, 4-oxopentanoate, 4-hydroxypentanoate, 3-oxoadipyl-CoA, 3-oxoadipate, 4-oxopentanoate, or 4-hydroxypentanoate. In certain embodiments, the microbial organisms of the invention do not include the production of a product other than butadiene, 1,3-butanediol, crotyl alcohol or 3-butene-2-ol, such as, but not limited to ethanol.

It is understood that any of the pathways disclosed herein, as described in the Examples and exemplified in the Figures, including the pathways of FIGS. 1-18, can be utilized to generate a non-naturally occurring microbial organism that produces any pathway intermediate or product, as desired. As disclosed herein, such a microbial organism that produces an intermediate can be used in combination with another microbial organism expressing downstream pathway enzymes to produce a desired product. However, it is understood that a non-naturally occurring microbial organism that produces a butadiene, 1,3-butanediol, crotyl alcohol or 3-buten-2-ol pathway intermediate can be utilized to produce the intermediate as a desired product.

The invention is described herein with general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The non-naturally occurring microbial organisms of the invention can be produced by introducing expressible nucleic acids encoding one or more of the enzymes or proteins participating in one or more butadiene, 1,3-butanediol, crotyl alcohol or 3-buten-2-ol biosynthetic pathways. Depending on the host microbial organism chosen for biosynthesis, nucleic acids for some or all of a particular butadiene, 1,3-butanediol, crotyl alcohol or 3-buten-2-ol biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve butadiene, 1,3-butanediol, crotyl alcohol or 3-buten-2-ol biosynthesis. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as butadiene, 1,3-butanediol, crotyl alcohol or 3-buten-2-ol.

Host microbial organisms can be selected from, and the non-naturally occurring microbial organisms generated in, for example, bacteria, yeast, fungus or any of a variety of other microorganisms applicable or suitable to fermentation processes. Exemplary bacteria include any species selected from the order Enterobacteriales, family Enterobacteriaceae, including the genera Escherichia and Klebsiella; the order Aeromonadales, family Succinivibrionaceae, including the genus Anaerobiospirillum; the order Pasteurellales, family Pasteurellaceae, including the genera Actinobacillus and Mannheimia; the order Rhizobiales, family Bradyrhizobiaceae, including the genus Rhizobium; the order Bacillales, family Bacillaceae, including the genus Bacillus; the order Actinomycetales, families Corynebacteriaceae and Streptomycetaceae, including the genus Corynebacterium and the genus Streptomyces, respectively; order Rhodospirillales, family Acetobacteraceae, including the genus Gluconobacter; the order Sphingomonadales, family Sphingomonadaceae, including the genus Zymomonas; the order Lactobacillales, families Lactobacillaceae and Streptococcaceae, including the genus Lactobacillus and the genus Lactococcus, respectively; the order Clostridiales, family Clostridiaceae, genus Clostridium; and the order Pseudomonadales, family Pseudomonadaceae, including the genus Pseudomonas. Non-limiting species of host bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida.

Similarly, exemplary species of yeast or fungi species include any species selected from the order Saccharomycetales, family Saccaromycetaceae, including the genera Saccharomyces, Kluyveromyces and Pichia; the order Saccharomycetales, family Dipodascaceae, including the genus Yarrowia; the order Schizosaccharomycetales, family Schizosaccaromycetaceae, including the genus Schizosaccharomyces; the order Eurotiales, family Trichocomaceae, including the genus Aspergillus; and the order Mucorales, family Mucoraceae, including the genus Rhizopus. Non-limiting species of host yeast or fungi include Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. E. coli is a particularly useful host organism since it is a well characterized microbial organism suitable for genetic engineering. Other particularly useful host organisms include yeast such as Saccharomyces cerevisiae. It is understood that any suitable microbial host organism can be used to introduce metabolic and/or genetic modifications to produce a desired product.

Depending on the butadiene, 1,3-butanediol, crotyl alcohol or 3-buten-2-ol biosynthetic pathway constituents of a selected host microbial organism, the non-naturally occurring microbial organisms of the invention will include at least one exogenously expressed butadiene, 1,3-butanediol, crotyl alcohol or 3-buten-2-ol pathway-encoding nucleic acid and up to all encoding nucleic acids for one or more butadiene, 1,3-butanediol, crotyl alcohol or 3-buten-2-ol biosynthetic pathways. For example, butadiene, 1,3-butanediol, crotyl alcohol or 3-buten-2-ol biosynthesis can be established in a host deficient in a pathway enzyme or protein through exogenous expression of the corresponding encoding nucleic acid. In a host deficient in all enzymes or proteins of a butadiene, 1,3-butanediol, crotyl alcohol or 3-buten-2-ol pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of butadiene, 1,3-butanediol, crotyl alcohol or 3-buten-2-ol can be included, such as steps 1B, 1C, 1F, 1G and 1Q in combination with any one of steps 1T, 10AS, 10P, 10N, 10AA, 15A, 15B, 15C, and 15G; or steps 10AT, 10P, 10N, 10AA, 15A, 15B, 15C, and 15G; or steps 14A, 14B, 14C, 14D, 14E, 13A, and 13B; or steps 17A, 17B, 17C, 17D, and 17G; or steps 17A, 17E, 17F, 17D, and 17G; or steps 18A, 18B, 18C, 18D, 18E, and 18F; or steps 1T, 10AS, 10P, 10AB, 10V, 10AH, 11A, 11B, and 11C; or steps 10AT, 10P, 10AB, 10V, 10AH, 11A, 11B, and 11C; or steps 13A and 13B; or steps 1T, 10AS, 10P, 10AB, 10V, and 10AH; 10AS, 10P, 10AB, 10AF, 10AG, and 10AH; or steps 1T, 10AS, 10P, 10AB, and 10W; or steps 10AT, 10P, 10AB, 10V, and 10AH; or steps 10AT, 10P, 10AB, 10AF, 10AG, and 10AH; or steps 10AT, 10P, 10AB, and 10W; or steps 1T, 10AS, 10P, 10N, and 10AA; or steps 1T, 10AS, 10P, 10Y, 10Z, and 10AA; or steps 10AT, 10P, 10N, and 10AA; or steps 10AT, 10P, 10Y, 10Z, and 10AA; or steps 10AS, 10P, 10N, 10AA, 15A, 15B, and 15C; or steps 10AT, 10P, 10N, 10AA, 15A, 15B; or steps 14A, 14B, 14C, 14D, 14E, and 13A; or steps 17A, 17B, 17C, and 17D; or steps 17A, 17E, 17F, and 17D; or steps 18A, 18B, 18C, 18D, and 18E, as depicted in FIGS. 1, and 10-18.

Given the teachings and guidance provided herein, those skilled in the art will understand that the number of encoding nucleic acids to introduce in an expressible form will, at least, parallel the butadiene, 1,3-butanediol, crotyl alcohol or 3-buten-2-ol pathway deficiencies of the selected host microbial organism. Therefore, a non-naturally occurring microbial organism of the invention can have one, two, three, four, five, six, seven, eight, nine, ten, eleven, twelve, thirteen, fourteen, fifteen, sixteen, seventeen, eighteen, nineteen, twenty up to all nucleic acids encoding the enzymes or proteins constituting a butadiene, 1,3-butanediol, crotyl alcohol or 3-buten-2-ol biosynthetic pathway disclosed herein. In some embodiments, the non-naturally occurring microbial organisms also can include other genetic modifications that facilitate or optimize butadiene, 1,3-butanediol, crotyl alcohol or 3-buten-2-ol biosynthesis or that confer other useful functions onto the host microbial organism. One such other functionality can include, for example, augmentation of the synthesis of one or more of the butadiene, 1,3-butanediol, crotyl alcohol or 3-buten-2-ol pathway precursors such as pyruvate, formate, acetyl-CoA, acetoacetyl-CoA, malonyl-CoA, malonyl-ACP, acetoacetyl-CoA, and succinyl-CoA.

Generally, a host microbial organism is selected such that it produces the precursor of a butadiene, 1,3-butanediol, crotyl alcohol or 3-buten-2-ol pathway, either as a naturally produced molecule or as an engineered product that either provides de novo production of a desired precursor or increased production of a precursor naturally produced by the host microbial organism. For example, pyruvate, formate, acetyl-CoA, acetoacetyl-CoA, malonyl-CoA, malonyl-ACP, acetoacetyl-CoA, and succinyl-CoA are produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of a butadiene, 1,3-butanediol, crotyl alcohol or 3-buten-2-ol pathway.

In some embodiments, a non-naturally occurring microbial organism of the invention is generated from a host that contains the enzymatic capability to synthesize butadiene, 1,3-butanediol, crotyl alcohol or 3-buten-2-ol. In this specific embodiment it can be useful to increase the synthesis or accumulation of a butadiene, 1,3-butanediol, crotyl alcohol or 3-buten-2-ol pathway product to, for example, drive butadiene, 1,3-butanediol, crotyl alcohol or 3-buten-2-ol pathway reactions toward butadiene, 1,3-butanediol, crotyl alcohol or 3-buten-2-ol production. Increased synthesis or accumulation can be accomplished by, for example, overexpression of nucleic acids encoding one or more of the above-described butadiene, 1,3-butanediol, crotyl alcohol or 3-buten-2-ol pathway enzymes or proteins. Overexpression of the enzyme or enzymes and/or protein or proteins of the butadiene, 1,3-butanediol, crotyl alcohol or 3-buten-2-ol pathway can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing butadiene, 1,3-butanediol, crotyl alcohol or 3-buten-2-ol, through overexpression of one, two, three, four, five, six, seven, eight, nine, ten, eleven, twelve, thirteen, fourteen, fifteen, sixteen, seventeen, eighteen, nineteen, twenty, that is, up to all nucleic acids encoding butadiene, 1,3-butanediol, crotyl alcohol or 3-buten-2-ol biosynthetic pathway enzymes or proteins. In addition, a non-naturally occurring organism can be generated by mutagenesis of an endogenous gene that results in an increase in activity of an enzyme in the butadiene, 1,3-butanediol, crotyl alcohol or 3-buten-2-ol biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods of the invention, any of the one or more exogenous nucleic acids can be introduced into a microbial organism to produce a non-naturally occurring microbial organism of the invention. The nucleic acids can be introduced so as to confer, for example, a butadiene, 1,3-butanediol, crotyl alcohol or 3-buten-2-ol biosynthetic pathway onto the microbial organism. Alternatively, encoding nucleic acids can be introduced to produce an intermediate microbial organism having the biosynthetic capability to catalyze some of the required reactions to confer butadiene, 1,3-butanediol, crotyl alcohol or 3-buten-2-ol biosynthetic capability. For example, a non-naturally occurring microbial organism having a butadiene, 1,3-butanediol, crotyl alcohol or 3-buten-2-ol biosynthetic pathway can comprise at least two exogenous nucleic acids encoding desired enzymes or proteins, such as the combination of a formate reductase and a 3-buten-2-ol dehydratase, or alternatively, a methanol dehydrogenase and crotyl alcohol dehydratase, or alternatively a formaldehyde dehydrogenase and a 3-hydroxybutyraldehyde reductase, and the like. Thus, it is understood that any combination of two or more enzymes or proteins of a biosynthetic pathway can be included in a non-naturally occurring microbial organism of the invention. Similarly, it is understood that any combination of three or more enzymes or proteins of a biosynthetic pathway can be included in a non-naturally occurring microbial organism of the invention, for example, a pyruvate formate lyase, a formyl-CoA reductase, and a crotonaldehyde reductase, or alternatively a formate dehydrogenase, a crotonyl-CoA reductase (aldehyde forming), and a crotonaldehyde reductase, or alternatively a 3-dexulose-6-phosphate synthase, a 6-phospho-3-hexuloisomerase, and acetoacetyl-CoA reductase (ketone reducing), and so forth, as desired, so long as the combination of enzymes and/or proteins of the desired biosynthetic pathway results in production of the corresponding desired product. Similarly, any combination of four, five, six, seven, eight, nine, ten, eleven, twelve, thirteen, fourteen, fifteen, sixteen, seventeen, eighteen, nineteen, twenty or more enzymes or proteins of a biosynthetic pathway as disclosed herein can be included in a non-naturally occurring microbial organism of the invention, as desired, so long as the combination of enzymes and/or proteins of the desired biosynthetic pathway results in production of the corresponding desired product.

In addition to the biosynthesis of butadiene, 1,3-butanediol, crotyl alcohol or 3-buten-2-ol as described herein, the non-naturally occurring microbial organisms and methods of the invention also can be utilized in various combinations with each other and/or with other microbial organisms and methods well known in the art to achieve product biosynthesis by other routes. For example, one alternative to produce butadiene, 1,3-butanediol, crotyl alcohol or 3-buten-2-ol other than use of the butadiene, 1,3-butanediol, crotyl alcohol or 3-buten-2-ol producers is through addition of another microbial organism capable of converting a butadiene, 1,3-butanediol, crotyl alcohol or 3-buten-2-ol pathway intermediate to butadiene, 1,3-butanediol, crotyl alcohol or 3-buten-2-ol. One such procedure includes, for example, the fermentation of a microbial organism that produces a butadiene, 1,3-butanediol, crotyl alcohol or 3-buten-2-ol pathway intermediate. The butadiene, 1,3-butanediol, crotyl alcohol or 3-buten-2-ol pathway intermediate can then be used as a substrate for a second microbial organism that converts the butadiene, 1,3-butanediol, crotyl alcohol or 3-buten-2-ol pathway intermediate to butadiene, 1,3-butanediol, crotyl alcohol or 3-buten-2-ol. The butadiene, 1,3-butanediol, crotyl alcohol or 3-buten-2-ol pathway intermediate can be added directly to another culture of the second organism or the original culture of the butadiene, 1,3-butanediol, crotyl alcohol or 3-buten-2-ol pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods of the invention can be assembled in a wide variety of subpathways to achieve biosynthesis of, for example, butadiene, 1,3-butanediol, crotyl alcohol or 3-buten-2-ol. In these embodiments, biosynthetic pathways for a desired product of the invention can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of butadiene, 1,3-butanediol, crotyl alcohol or 3-buten-2-ol can be accomplished by constructing a microbial organism that contains biosynthetic pathways for conversion of one pathway intermediate to another pathway intermediate or the product. Alternatively, butadiene, 1,3-butanediol, crotyl alcohol or 3-buten-2-ol also can be biosynthetically produced from microbial organisms through co-culture or co-fermentation using two organisms in the same vessel, where the first microbial organism produces a butadiene, 1,3-butanediol, crotyl alcohol or 3-buten-2-ol intermediate and the second microbial organism converts the intermediate to butadiene, 1,3-butanediol, crotyl alcohol or 3-buten-2-ol.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce butadiene, 1,3-butanediol, crotyl alcohol or 3-buten-2-ol.

Sources of encoding nucleic acids for a butadiene, 1,3-butanediol, crotyl alcohol or 3-buten-2-ol pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Abies grandis, Achromobacter xylosoxidans AXX-A, Acidaminococcus fermentans, Acinetobacter baylyi, Acinetobacter calcoaceticus, Acinetobacter sp. ADP1, Acinetobacter sp. Strain M-1, Allochromatium vinosum DSM 180, Amycolicicoccus subflavus DQS3-9A1, Anabaena variabilis ATCC 29413, Anaerotruncus colihominis, Aquincola tertiaricarbonis L108, Arabidopsis thaliana, Arabidopsis thaliana col, Archaeoglobus fulgidus, Archaeoglobus fulgidus DSM 4304, Arthrobacter globiformis, Aspergillus niger, Aspergillus terreus NIH2624, Azotobacter vinelandii DJ, Bacillus amyloliquefaciens, Bacillus cereus, Bacillus coahuilensis, Bacillus methanolicus MGA3, Bacillus methanolicus PB1, Bacillus pseudofirmus, Bacillus selenitireducens MLS10, Bacillus sphaericus, Bacillus subtilis, Bacteroides capillosus, Bordetella bronchiseptica KU1201, Bordetella bronchiseptica MO149, Bordetella parapertussis 12822, Bos taurus, Brassica napsus, Burkholderia ambifaria AMMD, Burkholderia phymatum, Burkholderia stabilis, Burkholderia xenovorans, Campylobacter curvus 525.92, Campylobacter jejuni, Candida albicans, Candida boidinii, Candida methylica, Candida parapsilosis, Candida tropicalis, Carboxydothermus hydrogenoformans, Carpoglyphus lactis, Carthamus tinctorius, Castellaniella defragrans, Chlamydomonas reinhardtii, Chlorobium phaeobacteroides DSM 266, Chloroflexus aurantiacus, Citrobacter freundii, Citrobacter koseri ATCC BAA-895, Citrobacter youngae ATCC 29220, Clostridium acetobutylicum, Clostridium acetobutylicum ATCC 824, Clostridium acidurici, Clostridium aminobutyricum, Clostridium beijerinckii, Clostridium beijerinckii NRRL B593, Clostridium botulinum, Clostridium botulinum C str. Eklund, Clostridium butyricum, Clostridium carboxidivorans P7, Clostridium cellulolyticum H10, Clostridium cellulovorans 743B, Clostridium kluyveri, Clostridium kluyveri DSM 555, Clostridium ljungdahlii, Clostridium ljungdahlii DSM 13528, Clostridium novyi NT, Clostridium pasteuranum, Clostridium perfringens, Clostridium phytofermentans ISDg, Clostridium propionicum, Clostridium saccharoperbutylacetonicum, Comamonas sp. CNB-1, Corynebacterium glutamicum, Corynebacterium glutamicum ATCC 13032, Corynebacterium glutamicum ATCC 14067, Corynebacterium sp., Corynebacterium sp. U-96, Cryptosporidium parvum Iowa II, Cucumis sativus, Cuphea hookeriana, Cuphea palustris, Cupriavidus taiwanensis, Cyanobium PCC7001, Cyanothece sp. PCC 7424, Cyanothece sp. PCC 7425, Cyanothece sp. PCC 7822, Desulfatibacillum alkenivorans AK-01, Desulfitobacterium hafniense, Desulfovibrio africanus, Desulfovibrio desulfuricans subsp. desulfuricans str. ATCC 27774, Desulfovibrio fructosovorans JJ, Dictyostelium discoideum AX4, Elizabethkingia meningoseptica, Enterococcus faecalis, Erythrobacter sp. NAPE Escherichia coli C, Escherichia coli K12, Escherichia coli K-12 MG1655, Escherichia coli W, Eubacterium barkeri, Eubacterium rectale ATCC 33656, Euglena gracilis, Fusobacterium nucleatum, Geobacillus thermoglucosidasius, Geobacter metallireducens GS-15, Geobacter sulfurreducens, Geobacter sulfurreducens PCA, Haematococcus pluvialis, Haliangium ochraceum DSM 14365, Haloarcula marismortui, Haloarcula marismortui ATCC 43049, Helicobacter pylori, Homo sapiens, Hydrogenobacter thermophilus, Hyphomicrobium denitrificans ATCC 51888, Hyphomicrobium zavarzinii, Jeotgalicoccus sp. ATCC8456, Klebsiella oxytoca, Klebsiella pneumonia, Klebsiella pneumonia ATCC 25955, Klebsiella pneumonia IAM1063, Klebsiella pneumoniae, Klebsiella terrigena, Kluyveromyces lactis, Lactobacillus acidophilus, Lactobacillus brevis ATCC 367, Lactobacillus collinoides, Lactobacillus plantarum, Lactococcus lactis, Leuconostoc mesenteroides, Lycopersicon hirsutum f glabratum, Lyngbya majuscule 3L, Lyngbya sp. PCC 8106, Lysinibacillus fusiformis, Lysinibacillus sphaericus, Macrococcus caseolyticus, Malus×domestica, marine gamma proteobacterium HTCC2080, Mesorhizobium loti MAFF303099, Metallosphaera sedula, Metarhizium acridum CQMa 102, Methanocaldococcus jannaschii, Methanosarcina acetivorans, Methanosarcina barkeri, Methanosarcina mazei, Methanothermobacter thermautotrophicus, Methylibium petroleiphilum PM1, Methylobacter marinus, Methylobacterium extorquens, Methylobacterium extorquens AM1, Methylococcus capsulatas, Methylococcus capsulatis, Methylomonas aminofaciens, Moorella thermoacetica, Mus musculus, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis MC2 155, Mycobacterium tuberculosis, Mycoplasma pneumoniae M129, Natranaerobius thermophilus, Nectria haematococca mpVI 77-13-4, Neurospora crassa, Nicotiana tabacum, Nocardia brasiliensis, Nocardia farcinica IFM 10152, Nocardia iowensis, Nocardia iowensis (sp. NRRL 5646), Nodularia spumigena CCY9414, Nostoc azollae, Nostoc sp. PCC 7120, Ocimum basilicum, Ogataea parapolymorpha DL-1 (Hansenula polymorpha DL-1), Oryctolagus cuniculus, Oxalobacter formigenes, Paenibacillus polymyxa, Paracoccus denitrificans, Pelobacter carbinolicus DSM 2380, Pelotomaculum therm opropionicum, Penicillium chrysogenum, Perkinsus marinus ATCC 50983, Picea abies, Pichia pastoris, Pinus sabiniana, Plasmodium falciparum, Populus alba, Populus tremula×Populus alba, Porphyromonas gingivalis, Porphyromonas gingivalis ATCC 33277, Porphyromonas gingivalis W83, Prochlorococcus marinus MIT 9312, Pseudomonas aeruginosa, Pseudomonas aeruginosa PAO1, Pseudomonas fluorescens, Pseudomonas fragi, Pseudomonas knackmussii, Pseudomonas knackmussii (B13), Pseudomonas mendocina, Pseudomonas putida, Pseudomonas sp, Psychroflexus torquis ATCC 700755, Pueraria montana, Pyrobaculum aerophilum str. IM2, Pyrococcus abyssi, Pyrococcus furiosus, Pyrococcus horikoshii OT3, Ralstonia eutropha, Ralstonia eutropha H16, Ralstonia metallidurans, Ralstonia pickettii, Rattus norvegicus, Rhizobium leguminosarum, Rhodobacter capsulatus, Rhodobacter sphaeroides, Rhodobacter sphaeroides ATCC 17025, Rhodococcus opacus B4, Rhodococcus ruber, Rhodopseudomonas palustris, Rhodopseudomonas palustris CGA009, Rhodospirillum rubrum, Roseburia intestinalis L1-82, Roseburia inulinivorans, Roseburia sp. A2-183, Roseiflexus castenholzii, Rubrivivax gelatinosus, Saccharomyces cerevisiae, Saccharomyces cerevisiae S288c, Salmonella enterica, Salmonella enterica subsp. arizonae serovar, Salmonella enterica subsp. enterica serovar Typhimurium str. LT2, Salmonella enterica Typhimurium, Salmonella typhimurium, Salmonella typhimurium LT2, Schizosaccharomyces pombe, Simmondsia chinensis, Sinorhizobium meliloti 1021, Solanum lycopersicum, Solibacillus silvestris, Sporosarcina newyorkensis, Staphylococcus aureus, Staphylococcus pseudintermedius, Stereum hirsutum FP-91666 SSE Streptococcus mutans, Streptococcus pneumoniae, Streptococcus pyogenes ATCC 10782, Streptomyces anulatus, Streptomyces avermitillis, Streptomyces cinnamonensis, Streptomyces coelicolor, Streptomyces griseus, Streptomyces griseus subsp. griseus NBRC 13350, Streptomyces sp CL190, Streptomyces sp. ACT-1, Streptomyces sp. KO-3988, Sulfolobus acidocalarius, Sulfolobus shibatae, Sulfolobus solfataricus, Sulfolobus tokodaii, Synechococcus elongatus PCC 6301, Synechococcus elongatus PCC7942, Synechococcus sp. PCC 7002, Synechocystis str. PCC 6803, Syntrophobacter fumaroxidans, Syntrophus aciditrophicus, Thauera aromatica, Thermoanaerobacter brockii HTD4, Thermoanaerobacter tengcongensis MB4, Thermococcus kodakaraensis, Thermococcus litoralis, Thermomyces lanuginosus, Thermoproteus neutrophilus, Thermotoga maritime MSB8, Thermus thermophilus, Thiocapsa roseopersicina, Trichomonas vaginalis G3, Trypsonoma brucei, Tsukamurella paurometabola DSM 20162, Umbellularia californica, Xanthobacter autotrophicus Py2, Yarrowia lipolytica, Yersinia intermedia ATCC 29909, Zea mays, Zoogloea ramigera, Zymomonas mobilis, as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite butadiene, 1,3-butanediol, crotyl alcohol or 3-buten-2-ol biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of butadiene, 1,3-butanediol, crotyl alcohol or 3-buten-2-ol described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative butadiene, 1,3-butanediol, crotyl alcohol or 3-buten-2-ol biosynthetic pathway exists in an unrelated species, butadiene, 1,3-butanediol, crotyl alcohol or 3-buten-2-ol biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize butadiene, 1,3-butanediol, crotyl alcohol or 3-buten-2-ol.

Methods for constructing and testing the expression levels of a non-naturally occurring butadiene, 1,3-butanediol, crotyl alcohol or 3-buten-2-ol-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of butadiene, 1,3-butanediol, crotyl alcohol or 3-buten-2-ol can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more butadiene, 1,3-butanediol, crotyl alcohol or 3-buten-2-ol biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In another aspect, provided herein is a method for producing butadiene comprising culturing the non-naturally occurring microbial organism of having a butadiene pathway as described herein under conditions and for a sufficient period of time to produce butadiene. In certain embodiments, the microbial organism has a formaldehyde fixation pathway, a formate assimilation pathway, a methanol metabolic pathway, a methanol oxidation pathway, a hydrogenase, a carbon monoxide dehydrogenase or any combination described herein. In certain embodiments, the microbial organism comprises at least one exogenous nucleic acid encoding a butadiene pathway enzyme expressed in a sufficient amount to produce butadiene. In certain embodiments, the organism is cultured in a substantially anaerobic culture medium.

In another aspect, provided herein is a method for producing crotyl alcohol comprising culturing the non-naturally occurring microbial organism of having a crotyl alcohol pathway as described herein under conditions and for a sufficient period of time to produce crotyl alcohol. In certain embodiments, the microbial organism has a formaldehyde fixation pathway, a formate assimilation pathway, a methanol metabolic pathway, a methanol oxidation pathway, a hydrogenase, a carbon monoxide dehydrogenase or any combination described herein. In certain embodiments, the microbial organism comprises at least one exogenous nucleic acid encoding a crotyl alcohol pathway enzyme expressed in a sufficient amount to produce crotyl alcohol. In certain embodiments, the organism is cultured in a substantially anaerobic culture medium.

In another aspect, provided herein is a method for producing 1,3-butanediol comprising culturing the non-naturally occurring microbial organism of having a 1,3-butanediol pathway as described herein under conditions and for a sufficient period of time to produce 1,3-butanediol. In certain embodiments, the microbial organism has a formaldehyde fixation pathway, a formate assimilation pathway, a methanol metabolic pathway, a methanol oxidation pathway, a hydrogenase, a carbon monoxide dehydrogenase or any combination described herein. In certain embodiments, the microbial organism comprises at least one exogenous nucleic acid encoding a 1,3-butanediol pathway enzyme expressed in a sufficient amount to produce 1,3-butanediol. In certain embodiments, the organism is cultured in a substantially anaerobic culture medium.

In another aspect, provided herein is a method for producing 3-buten-2-ol comprising culturing the non-naturally occurring microbial organism of having a 3-buten-2-ol pathway as described herein under conditions and for a sufficient period of time to produce 3-buten-2-ol. In certain embodiments, the microbial organism has a formaldehyde fixation pathway, a formate assimilation pathway, a methanol metabolic pathway, a methanol oxidation pathway, a hydrogenase, a carbon monoxide dehydrogenase or any combination described herein. In certain embodiments, the microbial organism comprises at least one exogenous nucleic acid encoding a 3-buten-2-ol pathway enzyme expressed in a sufficient amount to produce 3-buten-2-ol. In certain embodiments, the organism is cultured in a substantially anaerobic culture medium.

In some embodiments, access to butadiene can be accomplished by biosynthetic production of crotyl alcohol and subsequent chemical dehydration to butadiene. In some embodiments, the invention provides a process for the production of butadiene that includes (a) culturing by fermentation in a sufficient amount of nutrients and media a non-naturally occurring microbial organism that produces crotyl alcohol as described herein; and (b) converting crotyl alcohol produced by culturing the non-naturally occurring microbial organism to butadiene. In some aspects, the converting crotyl alcohol to butadiene is performed by chemical dehydration in the presence of a catalyst.

In some embodiments, access to butadiene can be accomplished by biosynthetic production of 1,3-butanediol and subsequent chemical dehydration to butadiene. In some embodiments, the invention provides a process for the production of butadiene that includes (a) culturing by fermentation in a sufficient amount of nutrients and media a non-naturally occurring microbial organism that produces 1,3-butanediol as described herein; and (b) converting 1,3-butanediol produced by culturing the non-naturally occurring microbial organism to butadiene. In some aspects, the converting 1,3-butanediol to butadiene is performed by chemical dehydration in the presence of a catalyst.

In some embodiments, access to butadiene can be accomplished by biosynthetic production of 3-buten-2-ol and subsequent chemical dehydration to butadiene. In some embodiments, the invention provides a process for the production of butadiene that includes (a) culturing by fermentation in a sufficient amount of nutrients and media a non-naturally occurring microbial organism that produces 3-buten-2-ol as described herein; and (b) converting 3-buten-2-ol produced by culturing the non-naturally occurring microbial organism to butadiene. In some aspects, the converting 3-buten-2-ol to butadiene is performed by chemical dehydration in the presence of a catalyst.

Suitable purification and/or assays to test for the production of butadiene, 1,3-butanediol, crotyl alcohol or 3-buten-2-ol can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The butadiene, 1,3-butanediol, crotyl alcohol or 3-buten-2-ol can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products of the invention. For example, the butadiene, 1,3-butanediol, crotyl alcohol or 3-buten- 2-ol producers can be cultured for the biosynthetic production of butadiene, 1,3-butanediol, crotyl alcohol or 3-buten-2-ol. Accordingly, in some embodiments, the invention provides culture medium having the butadiene, 1,3-butanediol, crotyl alcohol or 3-buten-2-ol or butadiene, 1,3-butanediol, crotyl alcohol or 3-buten-2-ol pathway intermediate described herein. In some aspects, the culture mediums can also be separated from the non-naturally occurring microbial organisms of the invention that produced the butadiene, 1,3-butanediol, crotyl alcohol or 3-buten-2-ol or butadiene, 1,3-butanediol, crotyl alcohol or 3-buten-2-ol pathway intermediate. Methods for separating a microbial organism from culture medium are well known in the art. Exemplary methods include filtration, flocculation, precipitation, centrifugation, sedimentation, and the like.

For the production of butadiene, 1,3-butanediol, crotyl alcohol or 3-buten-2-ol, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high butadiene, 1,3-butanediol, crotyl alcohol or 3-buten-2-ol yields.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium, can include, for example, any carbohydrate source which can supply a source of carbon to the non-naturally occurring microorganism. Such sources include, for example, sugars such as glucose, xylose, arabinose, galactose, mannose, fructose, sucrose and starch; or glycerol, alone as the sole source of carbon or in combination with other carbon sources described herein or known in the art. In one embodiment, H2, CO, CO2 or any combination thereof can be supplied as the sole or supplemental feedstock to the other sources of carbon disclosed herein. In one embodiment, the carbon source is a sugar. In one embodiment, the carbon source is a sugar-containing biomass. In some embodiments, the sugar is glucose. In one embodiment, the sugar is xylose. In another embodiment, the sugar is arabinose. In one embodiment, the sugar is galactose. In another embodiment, the sugar is fructose. In other embodiments, the sugar is sucrose. In one embodiment, the sugar is starch. In certain embodiments, the carbon source is glycerol. In some embodiments, the carbon source is crude glycerol. In one embodiment, the carbon source is crude glycerol without treatment. In other embodiments, the carbon source is glycerol and glucose. In another embodiment, the carbon source is methanol and glycerol. In one embodiment, the carbon source is carbon dioxide. In one embodiment, the carbon source is formate. In one embodiment, the carbon source is methane. In one embodiment, the carbon source is methanol. In one embodiment, the carbon source is chemoelectro-generated carbon (see, e.g., Liao et al. (2012) Science 335:1596). In one embodiment, the chemoelectro-generated carbon is methanol. In one embodiment, the chemoelectro-generated carbon is formate. In one embodiment, the chemoelectro-generated carbon is formate and methanol. In one embodiment, the carbon source is a sugar and methanol. In another embodiment, the carbon source is a sugar and glycerol. In other embodiments, the carbon source is a sugar and crude glycerol. In yet other embodiments, the carbon source is a sugar and crude glycerol without treatment. In one embodiment, the carbon source is a sugar-containing biomass and methanol. In another embodiment, the carbon source is a sugar-containing biomass and glycerol. In other embodiments, the carbon source is a sugar-containing biomass and crude glycerol. In other embodiments, the carbon source is a methanol and crude glycerol. In other embodiments, the carbon source is a methanol and glycerol. In yet other embodiments, the carbon source is a sugar-containing biomass and crude glycerol without treatment. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms provided herein for the production of butadiene, 1,3-butanediol, crotyl alcohol or 3-buten-2-ol and other pathway intermediates.

In one embodiment, the carbon source is glycerol. In certain embodiments, the glycerol carbon source is crude glycerol or crude glycerol without further treatment. In a further embodiment, the carbon source comprises glycerol or crude glycerol, and also sugar or a sugar-containing biomass, such as glucose. In a specific embodiment, the concentration of glycerol in the fermentation broth is maintained by feeding crude glycerol, or a mixture of crude glycerol and sugar (e.g., glucose). In certain embodiments, sugar is provided for sufficient strain growth. In some embodiments, the sugar (e.g., glucose) is provided at a molar concentration ratio of glycerol to sugar of from 200:1 to 1:200. In some embodiments, the sugar (e.g., glucose) is provided at a molar concentration ratio of glycerol to sugar of from 100:1 to 1:100. In some embodiments, the sugar (e.g., glucose) is provided at a molar concentration ratio of glycerol to sugar of from 100:1 to 5:1. In some embodiments, the sugar (e.g., glucose) is provided at a molar concentration ratio of glycerol to sugar of from 50:1 to 5:1. In certain embodiments, the sugar (e.g., glucose) is provided at a molar concentration ratio of glycerol to sugar of 100:1. In one embodiment, the sugar (e.g., glucose) is provided at a molar concentration ratio of glycerol to sugar of 90:1. In one embodiment, the sugar (e.g., glucose) is provided at a molar concentration ratio of glycerol to sugar of 80:1. In one embodiment, the sugar (e.g., glucose) is provided at a molar concentration ratio of glycerol to sugar of 70:1. In one embodiment, the sugar (e.g., glucose) is provided at a molar concentration ratio of glycerol to sugar of 60:1. In one embodiment, the sugar (e.g., glucose) is provided at a molar concentration ratio of glycerol to sugar of 50:1. In one embodiment, the sugar (e.g., glucose) is provided at a molar concentration ratio of glycerol to sugar of 40:1. In one embodiment, the sugar (e.g., glucose) is provided at a molar concentration ratio of glycerol to sugar of 30:1. In one embodiment, the sugar (e.g., glucose) is provided at a molar concentration ratio of glycerol to sugar of 20:1. In one embodiment, the sugar (e.g., glucose) is provided at a molar concentration ratio of glycerol to sugar of 10:1. In one embodiment, the sugar (e.g., glucose) is provided at a molar concentration ratio of glycerol to sugar of 5:1. In one embodiment, the sugar (e.g., glucose) is provided at a molar concentration ratio of glycerol to sugar of 2:1. In one embodiment, the sugar (e.g., glucose) is provided at a molar concentration ratio of glycerol to sugar of 1:1. In certain embodiments, the sugar (e.g., glucose) is provided at a molar concentration ratio of glycerol to sugar of 1:100. In one embodiment, the sugar (e.g., glucose) is provided at a molar concentration ratio of glycerol to sugar of 1:90. In one embodiment, the sugar (e.g., glucose) is provided at a molar concentration ratio of glycerol to sugar of 1:80. In one embodiment, the sugar (e.g., glucose) is provided at a molar concentration ratio of glycerol to sugar of 1:70. In one embodiment, the sugar (e.g., glucose) is provided at a molar concentration ratio of glycerol to sugar of 1:60. In one embodiment, the sugar (e.g., glucose) is provided at a molar concentration ratio of glycerol to sugar of 1:50. In one embodiment, the sugar (e.g., glucose) is provided at a molar concentration ratio of glycerol to sugar of 1:40. In one embodiment, the sugar (e.g., glucose) is provided at a molar concentration ratio of glycerol to sugar of 1:30. In one embodiment, the sugar (e.g., glucose) is provided at a molar concentration ratio of glycerol to sugar of 1:20. In one embodiment, the sugar (e.g., glucose) is provided at a molar concentration ratio of glycerol to sugar of 1:10. In one embodiment, the sugar (e.g., glucose) is provided at a molar concentration ratio of glycerol to sugar of 1:5. In one embodiment, the sugar (e.g., glucose) is provided at a molar concentration ratio of glycerol to sugar of 1:2. In certain embodiments of the ratios provided above, the sugar is a sugar-containing biomass. In certain other embodiments of the ratios provided above, the glycerol is a crude glycerol or a crude glycerol without further treatment. In other embodiments of the ratios provided above, the sugar is a sugar-containing biomass, and the glycerol is a crude glycerol or a crude glycerol without further treatment.

Crude glycerol can be a by-product produced in the production of biodiesel, and can be used for fermentation without any further treatment. Biodiesel production methods include (1) a chemical method wherein the glycerol-group of vegetable oils or animal oils is substituted by low-carbon alcohols such as methanol or ethanol to produce a corresponding fatty acid methyl esters or fatty acid ethyl esters by transesterification in the presence of acidic or basic catalysts; (2) a biological method where biological enzymes or cells are used to catalyze transesterification reaction and the corresponding fatty acid methyl esters or fatty acid ethyl esters are produced; and (3) a supercritical method, wherein transesterification reaction is carried out in a supercritical solvent system without any catalysts. The chemical composition of crude glycerol can vary with the process used to produce biodiesel, the transesterification efficiency, recovery efficiency of the biodiesel, other impurities in the feedstock, and whether methanol and catalysts were recovered. For example, the chemical compositions of eleven crude glycerol collected from seven Australian biodiesel producers reported that glycerol content ranged between 38% and 96%, with some samples including more than 14% methanol and 29% ash. In certain embodiments, the crude glycerol comprises from 5% to 99% glycerol. In some embodiments, the crude glycerol comprises from 10% to 90% glycerol. In some embodiments, the crude glycerol comprises from 10% to 80% glycerol. In some embodiments, the crude glycerol comprises from 10% to 70% glycerol. In some embodiments, the crude glycerol comprises from 10% to 60% glycerol. In some embodiments, the crude glycerol comprises from 10% to 50% glycerol. In some embodiments, the crude glycerol comprises from 10% to 40% glycerol. In some embodiments, the crude glycerol comprises from 10% to 30% glycerol. In some embodiments, the crude glycerol comprises from 10% to 20% glycerol. In some embodiments, the crude glycerol comprises from 80% to 90% glycerol. In some embodiments, the crude glycerol comprises from 70% to 90% glycerol. In some embodiments, the crude glycerol comprises from 60% to 90% glycerol. In some embodiments, the crude glycerol comprises from 50% to 90% glycerol. In some embodiments, the crude glycerol comprises from 40% to 90% glycerol. In some embodiments, the crude glycerol comprises from 30% to 90% glycerol. In some embodiments, the crude glycerol comprises from 20% to 90% glycerol. In some embodiments, the crude glycerol comprises from 20% to 40% glycerol. In some embodiments, the crude glycerol comprises from 40% to 60% glycerol. In some embodiments, the crude glycerol comprises from 60% to 80% glycerol. In some embodiments, the crude glycerol comprises from 50% to 70% glycerol. In one embodiment, the glycerol comprises 5% glycerol. In one embodiment, the glycerol comprises 10% glycerol. In one embodiment, the glycerol comprises 15% glycerol. In one embodiment, the glycerol comprises 20% glycerol. In one embodiment, the glycerol comprises 25% glycerol. In one embodiment, the glycerol comprises 30% glycerol. In one embodiment, the glycerol comprises 35% glycerol. In one embodiment, the glycerol comprises 40% glycerol. In one embodiment, the glycerol comprises 45% glycerol. In one embodiment, the glycerol comprises 50% glycerol. In one embodiment, the glycerol comprises 55% glycerol. In one embodiment, the glycerol comprises 60% glycerol. In one embodiment, the glycerol comprises 65% glycerol. In one embodiment, the glycerol comprises 70% glycerol. In one embodiment, the glycerol comprises 75% glycerol. In one embodiment, the glycerol comprises 80% glycerol. In one embodiment, the glycerol comprises 85% glycerol. In one embodiment, the glycerol comprises 90% glycerol. In one embodiment, the glycerol comprises 95% glycerol. In one embodiment, the glycerol comprises 99% glycerol.

In one embodiment, the carbon source is methanol or formate. In certain embodiments, methanol is used as a carbon source in the formaldehyde assimilation pathways provided herein. In one embodiment, the carbon source is methanol or formate. In other embodiments, formate is used as a carbon source in the formaldehyde assimilation pathways provided herein. In specific embodiments, methanol is used as a carbon source in the methanol metabolic pathways provided herein, either alone or in combination with the product pathways provided herein.

In one embodiment, the carbon source comprises methanol, and sugar (e.g., glucose) or a sugar-containing biomass. In another embodiment, the carbon source comprises formate, and sugar (e.g., glucose) or a sugar-containing biomass. In one embodiment, the carbon source comprises methanol, formate, and sugar (e.g., glucose) or a sugar-containing biomass. In specific embodiments, the methanol or formate, or both, in the fermentation feed is provided as a mixture with sugar (e.g., glucose) or sugar-comprising biomass. In certain embodiments, sugar is provided for sufficient strain growth.

In certain embodiments, the carbon source comprises methanol and a sugar (e.g., glucose). In some embodiments, the sugar (e.g., glucose) is provided at a molar concentration ratio of methanol to sugar of from 200:1 to 1:200. In some embodiments, the sugar (e.g., glucose) is provided at a molar concentration ratio of methanol to sugar of from 100:1 to 1:100. In some embodiments, the sugar (e.g., glucose) is provided at a molar concentration ratio of methanol to sugar of from 100:1 to 5:1. In some embodiments, the sugar (e.g., glucose) is provided at a molar concentration ratio of methanol to sugar of from 50:1 to 5:1. In certain embodiments, the sugar (e.g., glucose) is provided at a molar concentration ratio of methanol to sugar of 100:1. In one embodiment, the sugar (e.g., glucose) is provided at a molar concentration ratio of methanol to sugar of 90:1. In one embodiment, the sugar (e.g., glucose) is provided at a molar concentration ratio of methanol to sugar of 80:1. In one embodiment, the sugar (e.g., glucose) is provided at a molar concentration ratio of methanol to sugar of 70:1. In one embodiment, the sugar (e.g., glucose) is provided at a molar concentration ratio of methanol to sugar of 60:1. In one embodiment, the sugar (e.g., glucose) is provided at a molar concentration ratio of methanol to sugar of 50:1. In one embodiment, the sugar (e.g., glucose) is provided at a molar concentration ratio of methanol to sugar of 40:1. In one embodiment, the sugar (e.g., glucose) is provided at a molar concentration ratio of methanol to sugar of 30:1. In one embodiment, the sugar (e.g., glucose) is provided at a molar concentration ratio of methanol to sugar of 20:1. In one embodiment, the sugar (e.g., glucose) is provided at a molar concentration ratio of methanol to sugar of 10:1. In one embodiment, the sugar (e.g., glucose) is provided at a molar concentration ratio of methanol to sugar of 5:1. In one embodiment, the sugar (e.g., glucose) is provided at a molar concentration ratio of methanol to sugar of 2:1. In one embodiment, the sugar (e.g., glucose) is provided at a molar concentration ratio of methanol to sugar of 1:1. In certain embodiments, the sugar (e.g., glucose) is provided at a molar concentration ratio of methanol to sugar of 1:100. In one embodiment, the sugar (e.g., glucose) is provided at a molar concentration ratio of methanol to sugar of 1:90. In one embodiment, the sugar (e.g., glucose) is provided at a molar concentration ratio of methanol to sugar of 1:80. In one embodiment, the sugar (e.g., glucose) is provided at a molar concentration ratio of methanol to sugar of 1:70. In one embodiment, the sugar (e.g., glucose) is provided at a molar concentration ratio of methanol to sugar of 1:60. In one embodiment, the sugar (e.g., glucose) is provided at a molar concentration ratio of methanol to sugar of 1:50. In one embodiment, the sugar (e.g., glucose) is provided at a molar concentration ratio of methanol to sugar of 1:40. In one embodiment, the sugar (e.g., glucose) is provided at a molar concentration ratio of methanol to sugar of 1:30. In one embodiment, the sugar (e.g., glucose) is provided at a molar concentration ratio of methanol to sugar of 1:20. In one embodiment, the sugar (e.g., glucose) is provided at a molar concentration ratio of methanol to sugar of 1:10. In one embodiment, the sugar (e.g., glucose) is provided at a molar concentration ratio of methanol to sugar of 1:5. In one embodiment, the sugar (e.g., glucose) is provided at a molar concentration ratio of methanol to sugar of 1:2. In certain embodiments of the ratios provided above, the sugar is a sugar-containing biomass.

In certain embodiments, the carbon source comprises formate and a sugar (e.g., glucose). In some embodiments, the sugar (e.g., glucose) is provided at a molar concentration ratio of formate to sugar of from 200:1 to 1:200. In some embodiments, the sugar (e.g., glucose) is provided at a molar concentration ratio of formate to sugar of from 100:1 to 1:100. In some embodiments, the sugar (e.g., glucose) is provided at a molar concentration ratio of formate to sugar of from 100:1 to 5:1. In some embodiments, the sugar (e.g., glucose) is provided at a molar concentration ratio of formate to sugar of from 50:1 to 5:1. In certain embodiments, the sugar (e.g., glucose) is provided at a molar concentration ratio of formate to sugar of 100:1. In one embodiment, the sugar (e.g., glucose) is provided at a molar concentration ratio of formate to sugar of 90:1. In one embodiment, the sugar (e.g., glucose) is provided at a molar concentration ratio of formate to sugar of 80:1. In one embodiment, the sugar (e.g., glucose) is provided at a molar concentration ratio of formate to sugar of 70:1. In one embodiment, the sugar (e.g., glucose) is provided at a molar concentration ratio of formate to sugar of 60:1. In one embodiment, the sugar (e.g., glucose) is provided at a molar concentration ratio of formate to sugar of 50:1. In one embodiment, the sugar (e.g., glucose) is provided at a molar concentration ratio of formate to sugar of 40:1. In one embodiment, the sugar (e.g., glucose) is provided at a molar concentration ratio of formate to sugar of 30:1. In one embodiment, the sugar (e.g., glucose) is provided at a molar concentration ratio of formate to sugar of 20:1. In one embodiment, the sugar (e.g., glucose) is provided at a molar concentration ratio of formate to sugar of 10:1. In one embodiment, the sugar (e.g., glucose) is provided at a molar concentration ratio of formate to sugar of 5:1. In one embodiment, the sugar (e.g., glucose) is provided at a molar concentration ratio of formate to sugar of 2:1. In one embodiment, the sugar (e.g., glucose) is provided at a molar concentration ratio of formate to sugar of 1:1. In certain embodiments, the sugar (e.g., glucose) is provided at a molar concentration ratio of formate to sugar of 1:100. In one embodiment, the sugar (e.g., glucose) is provided at a molar concentration ratio of formate to sugar of 1:90. In one embodiment, the sugar (e.g., glucose) is provided at a molar concentration ratio of formate to sugar of 1:80. In one embodiment, the sugar (e.g., glucose) is provided at a molar concentration ratio of formate to sugar of 1:70. In one embodiment, the sugar (e.g., glucose) is provided at a molar concentration ratio of formate to sugar of 1:60. In one embodiment, the sugar (e.g., glucose) is provided at a molar concentration ratio of formate to sugar of 1:50. In one embodiment, the sugar (e.g., glucose) is provided at a molar concentration ratio of formate to sugar of 1:40. In one embodiment, the sugar (e.g., glucose) is provided at a molar concentration ratio of formate to sugar of 1:30. In one embodiment, the sugar (e.g., glucose) is provided at a molar concentration ratio of formate to sugar of 1:20. In one embodiment, the sugar (e.g., glucose) is provided at a molar concentration ratio of formate to sugar of 1:10. In one embodiment, the sugar (e.g., glucose) is provided at a molar concentration ratio of formate to sugar of 1:5. In one embodiment, the sugar (e.g., glucose) is provided at a molar concentration ratio of formate to sugar of 1:2. In certain embodiments of the ratios provided above, the sugar is a sugar-containing biomass.

In certain embodiments, the carbon source comprises a mixture of methanol and formate, and a sugar (e.g., glucose). In certain embodiments, sugar is provided for sufficient strain growth. In some embodiments, the sugar (e.g., glucose) is provided at a molar concentration ratio of methanol and formate to sugar of from 200:1 to 1:200. In some embodiments, the sugar (e.g., glucose) is provided at a molar concentration ratio of methanol and formate to sugar of from 100:1 to 1:100. In some embodiments, the sugar (e.g., glucose) is provided at a molar concentration ratio of methanol and formate to sugar of from 100:1 to 5:1. In some embodiments, the sugar (e.g., glucose) is provided at a molar concentration ratio of methanol and formate to sugar of from 50:1 to 5:1. In certain embodiments, the sugar (e.g., glucose) is provided at a molar concentration ratio of methanol and formate to sugar of 100:1. In one embodiment, the sugar (e.g., glucose) is provided at a molar concentration ratio of methanol and formate to sugar of 90:1. In one embodiment, the sugar (e.g., glucose) is provided at a molar concentration ratio of methanol and formate to sugar of 80:1. In one embodiment, the sugar (e.g., glucose) is provided at a molar concentration ratio of methanol and formate to sugar of 70:1. In one embodiment, the sugar (e.g., glucose) is provided at a molar concentration ratio of methanol and formate to sugar of 60:1. In one embodiment, the sugar (e.g., glucose) is provided at a molar concentration ratio of methanol and formate to sugar of 50:1. In one embodiment, the sugar (e.g., glucose) is provided at a molar concentration ratio of methanol and formate to sugar of 40:1. In one embodiment, the sugar (e.g., glucose) is provided at a molar concentration ratio of methanol and formate to sugar of 30:1. In one embodiment, the sugar (e.g., glucose) is provided at a molar concentration ratio of methanol and formate to sugar of 20:1. In one embodiment, the sugar (e.g., glucose) is provided at a molar concentration ratio of methanol and formate to sugar of 10:1. In one embodiment, the sugar (e.g., glucose) is provided at a molar concentration ratio of methanol and formate to sugar of 5:1. In one embodiment, the sugar (e.g., glucose) is provided at a molar concentration ratio of methanol and formate to sugar of 2:1. In one embodiment, the sugar (e.g., glucose) is provided at a molar concentration ratio of methanol and formate to sugar of 1:1. In certain embodiments, the sugar (e.g., glucose) is provided at a molar concentration ratio of methanol and formate to sugar of 1:100. In one embodiment, the sugar (e.g., glucose) is provided at a molar concentration ratio of methanol and formate to sugar of 1:90. In one embodiment, the sugar (e.g., glucose) is provided at a molar concentration ratio of methanol and formate to sugar of 1:80. In one embodiment, the sugar (e.g., glucose) is provided at a molar concentration ratio of methanol and formate to sugar of 1:70. In one embodiment, the sugar (e.g., glucose) is provided at a molar concentration ratio of methanol and formate to sugar of 1:60. In one embodiment, the sugar (e.g., glucose) is provided at a molar concentration ratio of methanol and formate to sugar of 1:50. In one embodiment, the sugar (e.g., glucose) is provided at a molar concentration ratio of methanol and formate to sugar of 1:40. In one embodiment, the sugar (e.g., glucose) is provided at a molar concentration ratio of methanol and formate to sugar of 1:30. In one embodiment, the sugar (e.g., glucose) is provided at a molar concentration ratio of methanol and formate to sugar of 1:20. In one embodiment, the sugar (e.g., glucose) is provided at a molar concentration ratio of methanol and formate to sugar of 1:10. In one embodiment, the sugar (e.g., glucose) is provided at a molar concentration ratio of methanol and formate to sugar of 1:5. In one embodiment, the sugar (e.g., glucose) is provided at a molar concentration ratio of methanol and formate to sugar of 1:2. In certain embodiments of the ratios provided above, the sugar is a sugar-containing biomass.

In addition to renewable feedstocks such as those exemplified above, the butadiene, 1,3-butanediol, crotyl alcohol or 3-buten-2-ol microbial organisms of the invention also can be modified for growth on syngas as its source of carbon. In this specific embodiment, one or more proteins or enzymes are expressed in the butadiene, 1,3-butanediol, crotyl alcohol or 3-buten-2-ol producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2CO₂+4H₂+nADP+nPi→CH₃COOH+2H₂O+nATP

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyltetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a butadiene, 1,3-butanediol, crotyl alcohol or 3-buten-2-ol pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Additionally, the reductive (reverse) tricarboxylic acid cycle coupled with carbon monoxide dehydrogenase and/or hydrogenase activities can also be used for the conversion of CO, CO₂ and/or H₂ to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P)H:ferredoxin oxidoreductase, carbon monoxide dehydrogenase, and hydrogenase. Specifically, the reducing equivalents extracted from CO and/or H₂ by carbon monoxide dehydrogenase and hydrogenase are utilized to fix CO₂ via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to the butadiene, 1,3-butanediol, crotyl alcohol or 3-buten-2-ol precursors, glyceraldehyde-3-phosphate, phosphoenolpyruvate, and pyruvate, by pyruvate:ferredoxin oxidoreductase and the enzymes of gluconeogenesis. Following the teachings and guidance provided herein for introducing a sufficient number of encoding nucleic acids to generate a butadiene, 1,3-butanediol, crotyl alcohol or 3-buten-2-ol pathway, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the microbial organisms of the invention such that the modified organism contains a reductive TCA pathway can confer syngas utilization ability.

Accordingly, given the teachings and guidance provided herein, those skilled in the art will understand that a non-naturally occurring microbial organism can be produced that secretes the biosynthesized compounds of the invention when grown on a carbon source such as a carbohydrate. Such compounds include, for example, butadiene, 1,3-butanediol, crotyl alcohol or 3-buten-2-ol and any of the intermediate metabolites in the butadiene, 1,3-butanediol, crotyl alcohol or 3-buten-2-ol pathway. All that is required is to engineer in one or more of the required enzyme or protein activities to achieve biosynthesis of the desired compound or intermediate including, for example, inclusion of some or all of the butadiene, 1,3-butanediol, crotyl alcohol or 3-buten-2-ol biosynthetic pathways. Accordingly, the invention provides a non-naturally occurring microbial organism that produces and/or secretes butadiene, 1,3-butanediol, crotyl alcohol or 3-buten-2-ol when grown on a carbohydrate or other carbon source and produces and/or secretes any of the intermediate metabolites shown in the butadiene, 1,3-butanediol, crotyl alcohol or 3-buten-2-ol pathway when grown on a carbohydrate or other carbon source. The butadiene, 1,3-butanediol, crotyl alcohol or 3-buten-2-ol producing microbial organisms of the invention can initiate synthesis from an intermediate, for example, acetoacetyl-CoA, acetoacetate, 3-oxobutyraldehyde, acetoacetyl-ACP, acetoacetyl-CoA, acetoacetyl-ACP, acetoacetyl-CoA, 3-hydroxybutyryl-ACP, 3-hydroxybutyryl-ACP, 3-hydroxybutyryl-CoA, 3-hydroxybutyryl-CoA, acetoacetyl-CoA, acetoacetate, 3-oxobutyraldehyde, 4-hydroxy-2-butanone, crotonyl-ACP, crotonyl-CoA, 3-hydroxybutyryl-ACP, 3-hydroxybutyryl-CoA, 3-hydroxybutyrate, 3-hydroxybutyraldehyde, crotonaldehyde, crotonyl-ACP, crotonyl-CoA, crotonate, crotonaldehyde, 2-butenyl-4-phosphate, 2-butenyl-4-diphosphate, 3-oxoglutaryl-CoA, 3-hydroxy-5-oxopentanoate, 3,5-dihydroxy pentanoate, 3-hydroxy-5-phosphonatooxypentanoate, 3-hydroxy-5-[hydroxy(phosphonooxy)phosphoryl]oxy pentanoate, butenyl 4-biphosphate, 2-butenyl 4-diphosphate, 2-butanol, acetolactate, acetoin, 2,3-butanediol, 3-hydroxybutyryl phosphate, 3-hydroxybutyryl diphosphate, 3-oxopent-4-enoyl-CoA, 3-oxopent-4-enoate, 3-buten-2-one, 3-oxo-4-hydroxy pentanoyl-CoA, 3-oxo-4-hydroxy pentanoate, 3,4-dihydroxypentanoate, 3,4-dihydroxypentanoyl-CoA, 3,4-dihydroxypentanoate, 4-oxopentanoate, 4-hydroxypentanoate, 3-oxoadipyl-CoA, 3-oxoadipate, 4-oxopentanoate, or 4-hydroxypentanoate.

The non-naturally occurring microbial organisms of the invention are constructed using methods well known in the art as exemplified herein to exogenously express at least one nucleic acid encoding a butadiene, 1,3-butanediol, crotyl alcohol or 3-buten-2-ol pathway enzyme or protein in sufficient amounts to produce butadiene, 1,3-butanediol, crotyl alcohol or 3-buten-2-ol. It is understood that the microbial organisms of the invention are cultured under conditions sufficient to produce butadiene, 1,3-butanediol, crotyl alcohol or 3-buten-2-ol. Following the teachings and guidance provided herein, the non-naturally occurring microbial organisms of the invention can achieve biosynthesis of butadiene, 1,3-butanediol, crotyl alcohol or 3-buten-2-ol resulting in intracellular concentrations between about 0.1- 200 mM or more. Generally, the intracellular concentration of butadiene, 1,3-butanediol, crotyl alcohol or 3-buten-2-ol is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms of the invention.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the butadiene, 1,3-butanediol, crotyl alcohol or 3-buten-2-ol producers can synthesize butadiene, 1,3-butanediol, crotyl alcohol or 3-buten-2-ol at intracellular concentrations of 5-10 mM or more as well as all other concentrations exemplified herein. It is understood that, even though the above description refers to intracellular concentrations, butadiene, 1,3-butanediol, crotyl alcohol or 3-buten-2-ol producing microbial organisms can produce butadiene, 1,3-butanediol, crotyl alcohol or 3-buten-2-ol intracellularly and/or secrete the product into the culture medium.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g, toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

In addition to the culturing and fermentation conditions disclosed herein, growth condition for achieving biosynthesis of butadiene, 1,3-butanediol, crotyl alcohol or 3-buten-2-ol can include the addition of an osmoprotectant to the culturing conditions. In certain embodiments, the non-naturally occurring microbial organisms of the invention can be sustained, cultured or fermented as described herein in the presence of an osmoprotectant. Briefly, an osmoprotectant refers to a compound that acts as an osmolyte and helps a microbial organism as described herein survive osmotic stress. Osmoprotectants include, but are not limited to, betaines, amino acids, and the sugar trehalose. Non-limiting examples of such are glycine betaine, praline betaine, dimethylthetin, dimethylslfonioproprionate, 3-dimethylsulfonio-2-methylpropionate, pipecolic acid, dimethylsulfonioacetate, choline, L-carnitine and ectoine. In one aspect, the osmoprotectant is glycine betaine. It is understood to one of ordinary skill in the art that the amount and type of osmoprotectant suitable for protecting a microbial organism described herein from osmotic stress will depend on the microbial organism used. The amount of osmoprotectant in the culturing conditions can be, for example, no more than about 0.1 mM, no more than about 0.5 mM, no more than about 1.0 mM, no more than about 1.5 mM, no more than about 2.0 mM, no more than about 2.5 mM, no more than about 3.0 mM, no more than about 5.0 mM, no more than about 7.0 mM, no more than about 10 mM, no more than about 50 mM, no more than about 100 mM or no more than about 500 mM.

In some embodiments, the carbon feedstock and other cellular uptake sources such as phosphate, ammonia, sulfate, chloride and other halogens can be chosen to alter the isotopic distribution of the atoms present in butadiene, 1,3-butanediol, crotyl alcohol or 3-buten-2-ol or any butadiene, 1,3-butanediol, crotyl alcohol or 3-buten-2-ol pathway intermediate. The various carbon feedstock and other uptake sources enumerated above will be referred to herein, collectively, as “uptake sources.” Uptake sources can provide isotopic enrichment for any atom present in the product butadiene, 1,3-butanediol, crotyl alcohol or 3-buten-2-ol or butadiene, 1,3-butanediol, crotyl alcohol or 3-buten-2-ol pathway intermediate, or for side products generated in reactions diverging away from a butadiene, 1,3-butanediol, crotyl alcohol or 3-buten-2-ol pathway. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S−B)/(M−B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950)¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³.

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mile. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983)). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content and/or prepared downstream products that utilize of the invention having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, the present invention provides butadiene, 1,3-butanediol, crotyl alcohol or 3-buten-2-ol or a butadiene, 1,3-butanediol, crotyl alcohol or 3-buten-2-ol pathway intermediate that has a carbon-12, carbon-13, and carbon-14 ratio that reflects an atmospheric carbon, also referred to as environmental carbon, uptake source. For example, in some aspects the butadiene, 1,3-butanediol, crotyl alcohol or 3-buten-2-ol or a butadiene, 1,3-butanediol, crotyl alcohol or 3-buten-2-ol pathway intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, the present invention provides butadiene, 1,3-butanediol, crotyl alcohol or 3-buten-2-ol or a butadiene, 1,3-butanediol, crotyl alcohol or 3-buten-2-ol pathway intermediate that has a carbon-12, carbon-13, and carbon-14 ratio that reflects petroleum-based carbon uptake source. In this aspect, the butadiene, 1,3-butanediol, crotyl alcohol or 3-buten-2-ol or a butadiene, 1,3-butanediol, crotyl alcohol or 3-buten-2-ol pathway intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, the present invention provides butadiene, 1,3-butanediol, crotyl alcohol or 3-buten-2-ol or a butadiene, 1,3-butanediol, crotyl alcohol or 3-buten-2-ol pathway intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present invention relates to the biologically produced butadiene, 1,3-butanediol, crotyl alcohol or 3-buten-2-ol or butadiene, 1,3-butanediol, crotyl alcohol or 3-buten-2-ol pathway intermediate as disclosed herein, and to the products derived therefrom, wherein the butadiene, 1,3-butanediol, crotyl alcohol or 3-buten-2-ol or a butadiene, 1,3-butanediol, crotyl alcohol or 3-buten-2-ol pathway intermediate has a carbon-12, carbon-13, and carbon-14 isotope ratio of about the same value as the CO₂ that occurs in the environment. For example, in some aspects the invention provides bioderived butadiene, 1,3-butanediol, crotyl alcohol or 3-buten-2-ol or a bioderived butadiene, 1,3-butanediol, crotyl alcohol or 3-buten-2-ol intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived butadiene, 1,3-butanediol, crotyl alcohol or 3-buten-2-ol or a bioderived butadiene, 1,3-butanediol, crotyl alcohol or 3-buten-2-ol pathway intermediate as disclosed herein, wherein the bioderived product is chemically modified to generate a final product. Methods of chemically modifying a bioderived product of butadiene, 1,3-butanediol, crotyl alcohol or 3-buten-2-ol, or an intermediate thereof, to generate a desired product are well known to those skilled in the art, as described herein.

Butadiene is a chemical commonly used in many commercial and industrial applications. Provided herein are a bioderived butadiene and biobased products comprising one or more bioderived butadiene or bioderived butadiene intermediate produced by a non-naturally occurring microorganism of the invention or produced using a method disclosed herein. Also provided herein are uses for bioderived butadiene and the biobased products. Non-limiting examples are described herein and include the following. Biobased products comprising all or a portion of bioderived butadiene include polymers, including synthetic rubbers and ABS resins, and chemicals, including hexamethylenediamine (HMDA), 1,4-butanediol, tetrahydrofuran (THF), adiponitrile, lauryl lactam, caprolactam, chloroprene, sulfalone, n-octanol and octene-1. The biobased polymers, including co-polymers, and resins include those where butadiene is reacted with one or more other chemicals, such as other alkenes, e.g. styrene, to manufacture numerous copolymers, including acrylonitrile 1,3-butadiene styrene (ABS), styrene-1,3-butadiene rubber (styrene butadiene rubber; SBR), styrene-1,3-butadiene latex. Products comprising biobased butadiene in the form of polymer synthetic rubber (SBR) include synthetic rubber articles, including tires, adhesives, seals, sealants, coatings, hose and shoe soles, and in the form of synthetic rubber polybutadiene (polybutadiene rubber, PBR or BR) which is used in synthetic rubber articles including tires, seals, gaskets and adhesives and as an intermediate in production of thermoplastic resin including acrylonitrile-butadiene-styrene (ABS) and in production of high impact modifier of polymers such as high impact polystyrene (HIPS). ABS is used in molded articles, including pipe, telephone, computer casings, mobile phones, radios, and appliances. Other biobased BD polymers include a latex, including styrene-butadiene latex (SB), used for example in paper coatings, carpet backing, adhesives, and foam mattresses; nitrile rubber, used in for example hoses, fuel lines, gasket seals, gloves and footwear; and styrene-butadiene block copolymers, used for example in asphalt modifiers (for road and roofing construction applications), adhesives, footwear and toys. Chemical intermediates made from butadiene include adiponitrile, HMDA, lauryl lactam, and caprolactam, used for example in production of nylon, including nylon-6,6 and other nylon-6,X, and chloroprene used for example in production of polychloroprene (neoprene). Butanediol produced from butadiene is used for example in production of specialty polymer resins including thermoplastic including polybutylene terephthalate (PBT), used in molded articles including parts for automotive, electrical, water systems and small appliances. Butadiene is also a co-monomer for polyurethane and polyurethane-polyurea copolymers. Butadiene is a co-monomer for biodegradable polymers, including PBAT (poly(butylene adipate-co-terephthalate)) and PBS (poly(butylene succinate)). Tetrahydrofuran produced from butadiene finds use as a solvent and in production of elastic fibers. Conversion of butadiene to THF, and subsequently to polytetramethylene ether glycol (PTMEG) (also referred to as PTMO, polytetramethylene oxide and PTHF, poly(tetrahydrofuran)), provides an intermediate used to manufacture elastic fibers, e.g. spandex fiber, used in products such as LYCRA® fibers or elastane, for example when combined with polyurethane-polyurea copolymers. THF also finds use as an industrial solvent and in pharmaceutical production. PTMEG is also combined with in the production of specialty thermoplastic elastomers (TPE), including thermoplastic elastomer polyester (TPE-E or TPEE) and copolyester ethers (COPE). COPEs are high modulus elastomers with excellent mechanical properties and oil/environmental resistance, allowing them to operate at high and low temperature extremes. PTMEG and butadiene also make thermoplastic polyurethanes (e.g. TPE-U or TPEU) processed on standard thermoplastic extrusion, calendaring, and molding equipment, and are characterized by their outstanding toughness and abrasion resistance. Other biobased products of bioderived BD include styrene block copolymers used for example in bitumen modification, footwear, packaging, and molded extruded products; methylmethacrylate butadiene styrene and methacrylate butadiene styrene (MBS) resins—clear resins—used as impact modifier for transparent thermoplastics including polycarbonate (PC), polyvinyl carbonate (PVC) and poly)methyl methacrylate (PMMA); sulfalone used as a solvent or chemical; n-octanol and octene-1. Accordingly, in some embodiments, the invention provides a biobased product comprising one or more bioderived butadiene or bioderived butadiene intermediate produced by a non-naturally occurring microorganism of the invention or produced using a method disclosed herein.

Crotyl alcohol, also referred to as 2-buten-1-ol, is a valuable chemical intermediate. Crotyl alcohol is a chemical commonly used in many commercial and industrial applications. Non-limiting examples of such applications include production of crotyl halides, esters, and ethers, which in turn are chemical are chemical intermediates in the production of monomers, fine chemicals, such as sorbic acid, trimethylhydroquinone, crotonic acid and 3-methoxybutanol, agricultural chemicals, and pharmaceuticals. Exemplary fine chemical products include sorbic acid, trimethylhydroquinone, crotonic acid and 3-methoxybutanol. Crotyl alcohol is also a precursor to 1,3-butadiene. Crotyl alcohol is currently produced exclusively from petroleum feedstocks. For example Japanese Patent 47-013009 and U.S. Pat. Nos. 3,090,815, 3,090,816, and 3,542,883 describe a method of producing crotyl alcohol by isomerization of 1,2-epoxybutane. The ability to manufacture crotyl alcohol from alternative and/or renewable feedstocks would represent a major advance in the quest for more sustainable chemical production processes. Accordingly, in some embodiments, the invention provides a biobased monomer, fine chemical, agricultural chemical, or pharmaceutical comprising one or more bioderived crotyl alcohol or bioderived crotyl alcohol intermediate produced by a non-naturally occurring microorganism of the invention or produced using a method disclosed herein.

1,3-Butanediol is a chemical commonly used in many commercial and industrial applications. Non-limiting examples of such applications include its use as an organic solvent for food flavoring agents or as a hypoglycaemic agent and its use in the production of polyurethane and polyester resins. Moreover, optically active 1,3-butanediol is also used in the synthesis of biologically active compounds and liquid crystals. Still further, 1,3-butanediol can be used in commercial production of 1,3-butadiene, a compound used in the manufacture of synthetic rubbers (e.g., tires), latex, and resins. 1,3-butanediol can also be sued to synthesize (R)-3-hydroxybutyryl-(R)-1,3-butanediol monoester or (R)-3-ketobutyryl-(R)-1,3-butanediol. Accordingly, in some embodiments, the invention provides a biobased organic solvent, hypoglycaemic agent, polyurethane, polyester resin, synthetic rubber, latex, or resin comprising one or more bioderived 1,3-butanediol or bioderived 1,3-butanediol intermediate produced by a non-naturally occurring microorganism of the invention or produced using a method disclosed herein.

3-Buten-2-ol is a chemical commonly used in many commercial and industrial applications. Non-limiting examples of such applications include it use as a solvent, e.g. as a viscosity adjustor, a monomer for polymer production, or a precursor to a fine chemical such as in production of contrast agents for imaging (see US20110091374) or production of glycerol (see US20120302800A1). 3-Buten-2-ol can also be used as a precursor in the production of 1,3-butadiene. Accordingly, in some embodiments, the invention provides a biobased solvent, polymer (or plastic or resin made from that polymer), or fine chemical comprising one or more bioderived 3-buten-2-ol or bioderived 3-buten-2-ol intermediate produced by a non-naturally occurring microorganism of the invention or produced using a method disclosed herein.

Further, the present invention relates to the biologically produced butadiene, 1,3-butanediol, crotyl alcohol or 3-buten-2-ol or a pathway intermediate thereof as disclosed herein, and to the products derived therefrom, including non-biosynthetic enzymatic or chemical conversion of 1,3-butanediol, crotyl alcohol or 3-buten-2-ol to butadiene, wherein the butadiene, 1,3-butanediol, crotyl alcohol or 3-buten-2-ol or a pathway intermediate thereof has a carbon-12, carbon-13, and carbon-14 isotope ratio of about the same value as the CO₂ that occurs in the environment. For example, in some aspects the invention provides: bioderived butadiene, 1,3-butanediol, crotyl alcohol or 3-buten-2-ol or a pathway intermediate thereof having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived butadiene, 1,3-butanediol, crotyl alcohol or 3-buten-2-ol or a bioderived butadiene, 1,3-butanediol, crotyl alcohol or 3-buten-2-ol intermediate as disclosed herein, wherein the bioderived product is chemically modified to generate a final product. Methods of chemically modifying a bioderived product of butadiene, 1,3-butanediol, crotyl alcohol or 3-buten- 2-ol, or an intermediate thereof, to generate a desired product are well known to those skilled in the art, and are described herein. For each of the bioderived compounds described herein, the invention further provides a biobased product including biobased product and its uses as described herein, and further where the biobased product can have a carbon- 12 versus carbon-13 versus carbon-14 isotope ratio of about the same value as the CO₂ that occurs in the environment, and wherein the biobased product is generated directly from or in combination with bioderived butadiene, 1,3-butanediol, crotyl alcohol or 3-buten-2-ol, preferably bioderived butadiene made completely bio-synthetically or by enzymatic or chemical conversion of 1,3-butanediol, crotyl alcohol of 3-buten-2-ol to butadiene, or with bioderived butadiene, 1,3-butanediol, crotyl alcohol or 3-buten-2-ol intermediate as disclosed herein. Non-limiting examples of such biobased products include those described for each bioderived chemical, e.g. bioderived butadiene, including a plastic, thermoplastic, elastomer, polyester, polyurethane, polymer, co-polymer, synthetic rubber, resin, chemical, polymer intermediate, a molded product, a resin, organic solvent, hypoglycaemic agent, polyester resin, latex, monomer, fine chemical, agricultural chemical, pharmaceutical, cosmetic, personal care product, or perfume.

In some embodiments, the invention provides polymer, synthetic rubber, resin, or chemical comprising bioderived butadiene or bioderived butadiene pathway intermediate, wherein the bioderived butadiene or bioderived butadiene pathway intermediate includes all or part of the butadiene or butadiene pathway intermediate used in the production of polymer, synthetic rubber, resin, or chemical, or other biobased products described herein (for example hexamethylenediamine (HMDA), 1,4-butanediol, tetrahydrofuran (THF), adiponitrile, lauryl lactam, caprolactam, chloroprene, sulfalone, n-octanol, octene-1, ABS, SBR, PBR, PTMEG, COPE). Thus, in some aspects, the invention provides a biobased polymer, synthetic rubber, resin, or chemical or other biobased product described herein comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived butadiene or bioderived butadiene pathway intermediate as disclosed herein. Additionally, in some aspects, the invention provides a biobased polymer, synthetic rubber, resin, or chemical or other biobased product described herein (for example hexamethylenediamine (HMDA), 1,4-butanediol, tetrahydrofuran (THF), adiponitrile, lauryl lactam, caprolactam, chloroprene, sulfalone, n-octanol, octene-1, ABS, SBR, PBR, PTMEG, COPE), wherein the butadiene or butadiene pathway intermediate used in its production is a combination of bioderived and petroleum derived butadiene or butadiene pathway intermediate. For example, a biobased polymer, synthetic rubber, resin, or chemical or other biobased product described herein (for example hexamethylenediamine (HMDA), 1,4-butanediol, tetrahydrofuran (THF), adiponitrile, lauryl lactam, caprolactam, chloroprene, sulfalone, n-octanol, octene-1, ABS, SBR, PBR, PTMEG, COPE) can be produced using 50% bioderived butadiene and 50% petroleum derived butadiene or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the microbial organisms disclosed herein. It is understood that methods for producing polymer, synthetic rubber, resin, or chemical or other biobased product described herein (for example hexamethylenediamine (HMDA), 1,4-butanediol, tetrahydrofuran (THF), adiponitrile, lauryl lactam, caprolactam, chloroprene, sulfalone, n-octanol, octene-1, ABS, SBR, PBR, PTMEG, COPE) using the bioderived butadiene or bioderived butadiene pathway intermediate of the invention are well known in the art.

In some embodiments, the invention provides organic solvent, hypoglycaemic agent, polyurethane, polyester resin, synthetic rubber, latex, or resin comprising bioderived 1,3-butanediol or bioderived 1,3-butanediol pathway intermediate, wherein the bioderived 1,3-butanediol or bioderived 1,3-butanediol pathway intermediate includes all or part of the 1,3-butanediol or 1,3-butanediol pathway intermediate used in the production of organic solvent, hypoglycaemic agent, polyurethane, polyester resin, synthetic rubber, latex, or resin. Thus, in some aspects, the invention provides a biobased organic solvent, hypoglycaemic agent, polyurethane, polyester resin, synthetic rubber, latex, or resin comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived 1,3-butanediol or bioderived 1,3-butanediol pathway intermediate as disclosed herein. Additionally, in some aspects, the invention provides a biobased organic solvent, hypoglycaemic agent, polyurethane, polyester resin, synthetic rubber, latex, or resin wherein the 1,3-butanediol or 1,3-butanediol pathway intermediate used in its production is a combination of bioderived and petroleum derived 1,3-butanediol or 1,3-butanediol pathway intermediate. For example, a biobased organic solvent, hypoglycaemic agent, polyurethane, polyester resin, synthetic rubber, latex, or resin can be produced using 50% bioderived 1,3-butanediol and 50% petroleum derived 1,3-butanediol or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the microbial organisms disclosed herein. It is understood that methods for producing organic solvent, hypoglycaemic agent, polyurethane, polyester resin, synthetic rubber, latex, or resin using the bioderived 1,3-butanediol or bioderived 1,3-butanediol pathway intermediate of the invention are well known in the art.

In some embodiments, the invention provides monomer, fine chemical, agricultural chemical, or pharmaceutical comprising bioderived crotyl alcohol or bioderived crotyl alcohol pathway intermediate, wherein the bioderived crotyl alcohol or bioderived crotyl alcohol pathway intermediate includes all or part of the crotyl alcohol or crotyl alcohol pathway intermediate used in the production of monomer, fine chemical, agricultural chemical, or pharmaceutical. Thus, in some aspects, the invention provides a biobased monomer, fine chemical, agricultural chemical, or pharmaceutical comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived crotyl alcohol or bioderived crotyl alcohol pathway intermediate as disclosed herein. Additionally, in some aspects, the invention provides a biobased monomer, fine chemical, agricultural chemical, or pharmaceutical wherein the crotyl alcohol or crotyl alcohol pathway intermediate used in its production is a combination of bioderived and petroleum derived crotyl alcohol or crotyl alcohol pathway intermediate. For example, a biobased monomer, fine chemical, agricultural chemical, or pharmaceutical can be produced using 50% bioderived crotyl alcohol and 50% petroleum derived crotyl alcohol or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the microbial organisms disclosed herein. It is understood that methods for producing monomer, fine chemical, agricultural chemical, or pharmaceutical using the bioderived crotyl alcohol or bioderived crotyl alcohol pathway intermediate of the invention are well known in the art.

In some embodiments, the invention provides solvent (or solvent-containing composition), polymer (or plastic or resin made from that polymer), or a fine chemical, comprising bioderived 3-buten-2-ol or bioderived 3-buten- 2-ol pathway intermediate, wherein the bioderived 3-buten-2-ol or bioderived 3-buten-2-ol pathway intermediate includes all or part of the 3-buten-2-ol or 3-buten-2-ol pathway intermediate used in the production of the solvent (or composition containing the solvent), polymer (or plastic or resin made from that polymer) or fine chemical. Thus, in some aspects, the invention provides a biobased solvent (or composition containing the solvent), polymer (or plastic or resin made from that polymer) or fine chemical comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived 3-buten-2-ol or bioderived 3-buten-2-ol pathway intermediate as disclosed herein. Additionally, in some aspects, the invention provides the biobased solvent (or composition containing the solvent), polymer (or plastic or resin made from that polymer) or fine chemical wherein the 3-buten-2-ol or 3-buten-2-ol pathway intermediate used in its production is a combination of bioderived and petroleum derived 3-buten-2-ol or 3-buten-2-ol pathway intermediate. For example, the biobased the solvent (or composition containing the solvent), polymer (or plastic or resin made from that polymer) or fine chemical can be produced using 50% bioderived 3-buten-2-ol and 50% petroleum derived 3-buten-2-ol or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the microbial organisms disclosed herein. It is understood that methods for producing the solvent (or composition containing the solvent), polymer (or plastic or resin made from that polymer) or fine chemical using the bioderived 3-buten-2-ol or bioderived 3-buten-2-ol pathway intermediate of the invention are well known in the art.

As used herein, the term “bioderived” means derived from or synthesized by a biological organism and can be considered a renewable resource since it can be generated by a biological organism. Such a biological organism, in particular the microbial organisms of the invention disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a bioderived compound of the invention. A biobased or bioderived product is in contrast to a petroleum derived product, wherein such a product is derived from or synthesized from petroleum or a petrochemical feedstock.

In some embodiments, the invention provides a biobased product comprising bioderived butadiene, 1,3-butanediol, crotyl alcohol or 3-buten-2-ol or bioderived butadiene, 1,3-butanediol, crotyl alcohol or 3-buten-2-ol pathway intermediate, wherein the bioderived butadiene, 1,3-butanediol, crotyl alcohol or 3-buten-2-ol or bioderived butadiene, 1,3-butanediol, crotyl alcohol or 3-buten-2-ol pathway intermediate includes all or part of the butadiene, 1,3-butanediol, crotyl alcohol or 3-buten-2-ol or butadiene, 1,3-butanediol, crotyl alcohol or 3-buten-2-ol pathway intermediate used in the production of the biobased product. For example, the final biobased product can contain the bioderived butadiene, 1,3-butanediol, crotyl alcohol or 3-buten-2-ol, butadiene, 1,3-butanediol, crotyl alcohol or 3-buten-2-ol pathway intermediate, or a portion thereof that is the result of the manufacturing of biobased product. Such manufacturing can include chemically reacting the bioderived butadiene, 1,3-butanediol, crotyl alcohol or 3-buten-2-ol or bioderived butadiene, 1,3-butanediol, crotyl alcohol or 3-buten-2-ol pathway intermediate (e.g. chemical conversion, chemical functionalization, chemical coupling, oxidation, reduction, polymerization, copolymerization and the like) into the final biobased product. Thus, in some aspects, the invention provides a biobased product comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived butadiene, 1,3-butanediol, crotyl alcohol or 3-buten-2-ol or bioderived butadiene, 1,3-butanediol, crotyl alcohol or 3-buten-2-ol pathway intermediate as disclosed herein.

Additionally, in some embodiments, the invention provides a composition having a bioderived butadiene, 1,3-butanediol, crotyl alcohol or 3-buten-2-ol or butadiene, 1,3-butanediol, crotyl alcohol or 3-buten-2-ol pathway intermediate disclosed herein and a compound other than the bioderived butadiene, 1,3-butanediol, crotyl alcohol or 3-buten-2-ol or butadiene, 1,3-butanediol, crotyl alcohol or 3-buten-2-ol pathway intermediate. For example, in some aspects, the invention provides a biobased product wherein the butadiene, 1,3-butanediol, crotyl alcohol or 3-buten-2-ol or butadiene, 1,3-butanediol, crotyl alcohol or 3-buten-2-ol pathway intermediate used in its production is a combination of bioderived and petroleum derived butadiene, 1,3-butanediol, crotyl alcohol or 3-buten-2-ol or butadiene, 1,3-butanediol, crotyl alcohol or 3-buten-2-ol pathway intermediate. For example, a biobased product can be produced using 50% bioderived butadiene, 1,3-butanediol, crotyl alcohol or 3-buten-2-ol and 50% petroleum derived butadiene, 1,3-butanediol, crotyl alcohol or 3-buten-2-ol or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the microbial organisms disclosed herein. It is understood that methods for producing a biobased product using the bioderived butadiene, 1,3-butanediol, crotyl alcohol or 3-buten-2-ol or bioderived butadiene, 1,3-butanediol, crotyl alcohol or 3-buten-2-ol pathway intermediate of the invention are well known in the art.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of butadiene, 1,3-butanediol, crotyl alcohol or 3-buten-2-ol includes anaerobic culture or fermentation conditions. In certain embodiments, the non-naturally occurring microbial organisms of the invention can be sustained, cultured or fermented under anaerobic or substantially anaerobic conditions. Briefly, an anaerobic condition refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of butadiene, 1,3-butanediol, crotyl alcohol or 3-buten-2-ol. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of butadiene, 1,3-butanediol, crotyl alcohol or 3-buten-2-ol. Generally, and as with non-continuous culture procedures, the continuous and/or near-continuous production of butadiene, 1,3-butanediol, crotyl alcohol or 3-buten-2-ol will include culturing a non-naturally occurring butadiene, 1,3-butanediol, crotyl alcohol or 3-buten-2-ol producing organism of the invention in sufficient nutrients and medium to sustain and/or nearly sustain growth in an exponential phase. Continuous culture under such conditions can include, for example, growth for 1 day, 2, 3, 4, 5, 6 or 7 days or more. Additionally, continuous culture can include longer time periods of 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of butadiene, 1,3-butanediol, crotyl alcohol or 3-buten-2-ol can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the butadiene, 1,3-butanediol, crotyl alcohol or 3-buten-2-ol producers of the invention for continuous production of substantial quantities of butadiene, 1,3-butanediol, crotyl alcohol or 3-buten-2-ol, the butadiene, 1,3-butanediol, crotyl alcohol or 3-buten-2-ol producers also can be, for example, simultaneously subjected to chemical synthesis and/or enzymatic procedures to convert the product to other compounds or the product can be separated from the fermentation culture and sequentially subjected to chemical an/or enzymatic conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of butadiene, 1,3-butanediol, crotyl alcohol or 3-buten-2-ol.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene deletion or disruption strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

As disclosed herein, a nucleic acid encoding a desired activity of a butadiene, 1,3-butanediol, crotyl alcohol or 3-buten-2-ol pathway can be introduced into a host organism. In some cases, it can be desirable to modify an activity of a butadiene, 1,3-butanediol, crotyl alcohol or 3-buten-2-ol pathway enzyme or protein to increase production of butadiene, 1,3-butanediol, crotyl alcohol or 3-buten-2-ol. For example, known mutations that increase the activity of a protein or enzyme can be introduced into an encoding nucleic acid molecule. Additionally, optimization methods can be applied to increase the activity of an enzyme or protein and/or decrease an inhibitory activity, for example, decrease the activity of a negative regulator.

One such optimization method is directed evolution. Directed evolution is a powerful approach that involves the introduction of mutations targeted to a specific gene in order to improve and/or alter the properties of an enzyme. Improved and/or altered enzymes can be identified through the development and implementation of sensitive high-throughput screening assays that allow the automated screening of many enzyme variants (for example, >10⁴). Iterative rounds of mutagenesis and screening typically are performed to afford an enzyme with optimized properties. Computational algorithms that can help to identify areas of the gene for mutagenesis also have been developed and can significantly reduce the number of enzyme variants that need to be generated and screened. Numerous directed evolution technologies have been developed (for reviews, see Hibbert et al., Biomol. Eng 22:11-19 (2005); Huisman and Lalonde, In Biocatalysis in the pharmaceutical and biotechnology industries pgs. 717-742 (2007), Patel (ed.), CRC Press; Otten and Quax. Biomol. Eng 22:1-9 (2005); and Sen et al., Appl Biochem. Biotechnol 143:212-223 (2007)) to be effective at creating diverse variant libraries, and these methods have been successfully applied to the improvement of a wide range of properties across many enzyme classes. Enzyme characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening substrate binding to include non-natural substrates; inhibition (Kᵢ), to remove inhibition by products, substrates, or key intermediates; activity (kcat), to increases enzymatic reaction rates to achieve desired flux; expression levels, to increase protein yields and overall pathway flux; oxygen stability, for operation of air sensitive enzymes under aerobic conditions; and anaerobic activity, for operation of an aerobic enzyme in the absence of oxygen.

A number of exemplary methods have been developed for the mutagenesis and diversification of genes to target desired properties of specific enzymes. Such methods are well known to those skilled in the art. Any of these can be used to alter and/or optimize the activity of a butadiene, 1,3-butanediol, crotyl alcohol or 3-buten-2-ol pathway enzyme or protein. Such methods include, but are not limited to EpPCR, which introduces random point mutations by reducing the fidelity of DNA polymerase in PCR reactions (Pritchard et al., J Theor. Biol. 234:497-509 (2005)); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res. 32:e145 (2004); and Fujii et al., Nat. Protoc. 1:2493-2497 (2006)); DNA or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc Natl Acad Sci USA 91:10747-10751 (1994); and Stemmer, Nature 370:389-391 (1994)); Staggered Extension (StEP), which entails template priming followed by repeated cycles of 2 step PCR with denaturation and very short duration of annealing/extension (as short as 5 sec) (Zhao et al., Nat. Biotechnol. 16:258-261 (1998)); Random Priming Recombination (RPR), in which random sequence primers are used to generate many short DNA fragments complementary to different segments of the template (Shao et al., Nucleic Acids Res 26:681- 683 (1998)).

Additional methods include Heteroduplex Recombination, in which linearized plasmid DNA is used to form heteroduplexes that are repaired by mismatch repair (Volkov et al, Nucleic Acids Res. 27:e18 (1999); and Volkov et al., Methods Enzymol. 328:456-463 (2000)); Random Chimeragenesis on Transient Templates (RACHITT), which employs Dnase I fragmentation and size fractionation of single stranded DNA (ssDNA) (Coco et al., Nat. Biotechnol. 19:354-359 (2001)); Recombined Extension on Truncated templates (RETT), which entails template switching of unidirectionally growing strands from primers in the presence of unidirectional ssDNA fragments used as a pool of templates (Lee et al., J. Molec. Catalysis 26:119-129 (2003)); Degenerate Oligonucleotide Gene Shuffling (DOGS), in which degenerate primers are used to control recombination between molecules; (Bergquist and Gibbs, Methods Mol. Biol 352:191-204 (2007); Bergquist et al., Biomol. Eng 22:63-72 (2005); Gibbs et al., Gene 271:13-20 (2001)); Incremental Truncation for the Creation of Hybrid Enzymes (ITCHY), which creates a combinatorial library with 1 base pair deletions of a gene or gene fragment of interest (Ostermeier et al., Proc. Natl. Acad. Sci. USA 96:3562-3567 (1999); and Ostermeier et al., Nat. Biotechnol. 17:1205-1209 (1999)); Thio-Incremental Truncation for the Creation of Hybrid Enzymes (THIO-ITCHY), which is similar to ITCHY except that phosphothioate dNTPs are used to generate truncations (Lutz et al., Nucleic Acids Res 29:E16 (2001)); SCRATCHY, which combines two methods for recombining genes, ITCHY and DNA shuffling (Lutz et al., Proc. Natl. Acad. Sci. USA 98:11248-11253 (2001)); Random Drift Mutagenesis (RNDM), in which mutations made via epPCR are followed by screening/selection for those retaining usable activity (Bergquist et al., Biomol. Eng. 22:63-72 (2005)); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (Wong et al., Biotechnol. J. 3:74-82 (2008); Wong et al., Nucleic Acids Res. 32:e26 (2004); and Wong et al., Anal. Biochem. 341:187-189 (2005)); Synthetic Shuffling, which uses overlapping oligonucleotides designed to encode “all genetic diversity in targets” and allows a very high diversity for the shuffled progeny (Ness et al., Nat. Biotechnol. 20:1251-1255 (2002)); Nucleotide Exchange and Excision Technology NexT, which exploits a combination of dUTP incorporation followed by treatment with uracil DNA glycosylase and then piperidine to perform endpoint DNA fragmentation (Muller et al., Nucleic Acids Res. 33:e117 (2005)).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (Sieber et al., Nat. Biotechnol. 19:456-460 (2001)); Gene Site Saturation Mutagenesis™ (GSSM™), in which the starting materials include a supercoiled double stranded DNA (dsDNA) plasmid containing an insert and two primers which are degenerate at the desired site of mutations (Kretz et al., Methods Enzymol. 388:3-11 (2004)); Combinatorial Cassette Mutagenesis (CCM), which involves the use of short oligonucleotide cassettes to replace limited regions with a large number of possible amino acid sequence alterations (Reidhaar-Olson et al. Methods Enzymol. 208:564-586 (1991); and Reidhaar-Olson et al. Science 241:53-57 (1988)); Combinatorial Multiple Cassette Mutagenesis (CMCM), which is essentially similar to CCM and uses epPCR at high mutation rate to identify hot spots and hot regions and then extension by CMCM to cover a defined region of protein sequence space (Reetz et al., Angew. Chem. Int. Ed Engl. 40:3589-3591 (2001)); the Mutator Strains technique, in which conditional is mutator plasmids, utilizing the mutD5 gene, which encodes a mutant subunit of DNA polymerase III, to allow increases of 20 to 4000-X in random and natural mutation frequency during selection and block accumulation of deleterious mutations when selection is not required (Selifonova et al., Appl. Environ. Microbiol. 67:3645-3649 (2001)); Low et al., J. Mol. Biol. 260:359-3680 (1996)).

Additional exemplary methods include Look-Through Mutagenesis (LTM), which is a multidimensional mutagenesis method that assesses and optimizes combinatorial mutations of selected amino acids (Rajpal et al., Proc. Natl. Acad. Sci. USA 102:8466-8471 (2005)); Gene Reassembly, which is a DNA shuffling method that can be applied to multiple genes at one time or to create a large library of chimeras (multiple mutations) of a single gene (Tunable GeneReassembly™ (TGR™) Technology supplied by Verenium Corporation), in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (Hayes et al., Proc. Natl. Acad. Sci. USA 99:15926-15931 (2002)); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (Reetz et al., Nat. Protoc. 2:891-903 (2007); and Reetz et al., Angew. Chem. Int. Ed Engl. 45:7745-7751 (2006)).

Any of the aforementioned methods for mutagenesis can be used alone or in any combination. Additionally, any one or combination of the directed evolution methods can be used in conjunction with adaptive evolution techniques, as described herein.

It is understood that modifications which do not substantially affect the activity of the various embodiments of this invention are also provided within the definition of the invention provided herein. Accordingly, the following examples are intended to illustrate but not limit the present invention.

### Example I

### Formate Assimilation Pathways

This example describes enzymatic pathways for converting pyruvate to formaldehyde, and optionally in combination with producing acetyl-CoA and/or reproducing pyruvate.

### Step E, FIG. 1: Formate Reductase

The conversion of formate to formaldehyde can be carried out by a formate reductase (step E, FIG. 1). A suitable enzyme for these transformations is the aryl-aldehyde dehydrogenase, or equivalently a carboxylic acid reductase, from Nocardia iowensis. Carboxylic acid reductase catalyzes the magnesium, ATP and NADPH-dependent reduction of carboxylic acids to their corresponding aldehydes (Venkitasubramanian et al., J Biol. Chem. 282:478-485 (2007)). This enzyme, encoded by car, was cloned and functionally expressed in E. coli (Venkitasubramanian et al., J Biol. Chem. 282:478-485 (2007)). Expression of the npt gene product improved activity of the enzyme via post-transcriptional modification. The npt gene encodes a specific phosphopantetheine transferase (PPTase) that converts the inactive apo-enzyme to the active holo-enzyme. The natural substrate of this enzyme is vanillic acid, and the enzyme exhibits broad acceptance of aromatic and aliphatic substrates (Venkitasubramanian et al., in Biocatalysis in the Pharmaceutical and Biotechnology Industires, ed. R. N. Patel, Chapter 15, pp. 425-440, CRC Press LLC, Boca Raton, Fla. (2006)). Information related to these proteins and genes is shown below.

| Protein | GenBank ID | GI number | Organism |
|---|---|---|---|
| Car | AAR91681.1 | 40796035 | Nocardia iowensis |
| | | | (sp. NRRL 5646) |
| Npt | ABI83656.1 | 114848891 | Nocardia iowensis |
| | | | (sp. NRRL 5646) |

Additional car and npt genes can be identified based on sequence homology.

| Protein | GenBank ID | GI number | Organism |
|---|---|---|---|
| fadD9 | YP_978699.1 | 121638475 | Mycobacterium bovis BCG |
| BCG_2812c | YP_978898.1 | 121638674 | Mycobacterium bovis BCG |
| nfa20150 | YP_118225.1 | 54023983 | Nocardia farcinica IFM 10152 |
| nfa40540 | YP_120266.1 | 54026024 | Nocardia farcinica IFM 10152 |
| SGR_6790 | YP_001828302.1 | 182440583 | Streptomyces griseus subsp. griseus |
| | | | NBRC 13350 |
| SGR_665 | YP_001822177.1 | 182434458 | Streptomyces griseus subsp. griseus |
| | | | NBRC 13350 |
| MSMEG_2956 | YP_887275.1 | 118473501 | Mycobacterium smegmatis MC2 155 |
| MSMEG_5739 | YP_889972.1 | 118469671 | Mycobacterium smegmatis MC2 155 |
| MSMEG_2648 | YP_886985.1 | 118471293 | Mycobacterium smegmatis MC2 155 |
| MAP1040c | NP_959974.1 | 41407138 | Mycobacterium avium subsp. |
| | | | paratuberculosis K-10 |
| MAP2899c | NP_961833.1 | 41408997 | Mycobacterium avium subsp. |
| | | | paratuberculosis K-10 |
| MMAR_2117 | YP_001850422.1 | 183982131 | Mycobacterium marinum M |
| MMAR_2936 | YP_001851230.1 | 183982939 | Mycobacterium marinum M |
| MMAR_1916 | YP_001850220.1 | 183981929 | Mycobacterium marinum M |
| TpauDRAFT_33060 | ZP_04027864.1 | 227980601 | Tsukamurella paurometabola DSM 20162 |
| TpauDRAFT_20920 | ZP_04026660.1 | 227979396 | Tsukamurella paurometabola DSM 20162 |
| CPCC7001_1320 | ZP_05045132.1 | 254431429 | Cyanobium PCC7001 |
| DDBDRAFT_0187729 | XP_636931.1 | 66806417 | Dictyostelium discoideum AX4 |

An additional enzyme candidate found in Streptomyces griseus is encoded by the griC and griD genes. This enzyme is believed to convert 3-amino-4-hydroxybenzoic acid to 3-amino-4-hydroxybenzaldehyde as deletion of either griC or griD led to accumulation of extracellular 3-acetylamino-4-hydroxybenzoic acid, a shunt product of 3-amino-4-hydroxybenzoic acid metabolism (Suzuki, et al., J Antibiot. 60(6):380-387 (2007)). Co-expression of griC and griD with SGR_{—}665, an enzyme similar in sequence to the Nocardia iowensis npt, can be beneficial. Information related to these proteins and genes is shown below.

| Protein | GenBank ID | GI number | Organism |
|---|---|---|---|
| griC | YP_001825755.1 | 182438036 | Streptomyces griseus subsp. griseus NBRC 13350 |
| grid | YP_001825756.1 | 182438037 | Streptomyces griseus subsp. griseus NBRC 13350 |

An enzyme with similar characteristics, alpha-aminoadipate reductase (AAR, EC 1.2.1.31), participates in lysine biosynthesis pathways in some fungal species. This enzyme naturally reduces alpha-aminoadipate to alpha-aminoadipate semialdehyde. The carboxyl group is first activated through the ATP-dependent formation of an adenylate that is then reduced by NAD(P)H to yield the aldehyde and AMP. Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279 (2003)), and Schizosaccharomyces pombe (Ford et al., Curr. Genet. 28:131-137 (1995)). The AAR from S. pombe exhibited significant activity when expressed in E. coli (Guo et al., Yeast 21:1279-1288 (2004)). The AAR from Penicillium chrysogenum accepts S-carboxymethyl-L-cysteine as an alternate substrate, but did not react with adipate, L-glutamate or diaminopimelate (Hijarrubia et al., J. Biol. Chem. 278:8250-8256 (2003)). The gene encoding the P. chrysogenum PPTase has not been identified to date. Information related to these proteins and genes is shown below.

| Protein | GenBank ID | GI number | Organism |
|---|---|---|---|
| LYS2 | AAA34747.1 | 171867 | Saccharomyces cerevisiae |
| LYS5 | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Penicillium chrysogenum |

Tani et al (Agric Biol Chem, 1978, 42: 63-68; Agric Biol Chem, 1974, 38: 2057-2058) showed that purified enzymes from Escherichia coli strain B could reduce the sodium salts of different organic acids (e.g. formate, glycolate, acetate, etc.) to their respective aldehydes (e.g. formaldehyde, glycoaldehyde, acetaldehyde, etc.). Of three purified enzymes examined by Tani et al (1978), only the “A” isozyme was shown to reduce formate to formaldehyde. Collectively, this group of enzymes was originally termed glycoaldehyde dehydrogenase; however, their novel reductase activity led the authors to propose the name glycolate reductase as being more appropriate (Morita et al, Agric Biol Chem, 1979, 43: 185-186). Morita et al (Agric Biol Chem, 1979, 43: 185-186) subsequently showed that glycolate reductase activity is relatively widespread among microorganisms, being found for example in: Pseudomonas, Agrobacterium, Escherichia, Flavobacterium, Micrococcus, Staphylococcus, Bacillus, and others. Without wishing to be bound by any particular theory, it is believed that some of these glycolate reductase enzymes are able to reduce formate to formaldehyde.

Any of these CAR or CAR-like enzymes can exhibit formate reductase activity or can be engineered to do so.

### Step F, Figure Formate Ligase, Formate Transferase, Formate Synthetase

The acylation of formate to formyl-CoA is catalyzed by enzymes with formate transferase, synthetase, or ligase activity (Step F, FIG. 1). Formate transferase enzymes have been identified in several organisms including Escherichia coli (Toyota, et al., J Bacteriol. 2008 April; 190(7):2556-64), Oxalobacter formigenes (Toyota, et al., J Bacteriol. 2008 April; 190(7):2556-64; Baetz et al., J Bacteriol. 1990 July; 172(7):3537-40; Ricagno, et al., EMBO J 2003 Jul. 1; 22(13):3210-9)), and Lactobacillus acidophilus (Azcarate-Peril, et al., Appl. Environ. Microbiol. 2006 72(3) 1891-1899). Homologs exist in several other organisms. Enzymes acting on the CoA-donor for formate transferase may also be expressed to ensure efficient regeneration of the CoA-donor. For example, if oxalyl-CoA is the CoA donor substrate for formate transferase, an additional transferase, synthetase, or ligase may be required to enable efficient regeneration of oxalyl-CoA from oxalate. Similarly, if succinyl-CoA or acetyl-CoA is the CoA donor substrate for formate transferase, an additional transferase, synthetase, or ligase may be required to enable efficient regeneration of succinyl-CoA from succinate or acetyl-CoA from acetate, respectively.

| Protein | GenBank ID | GI number | Organism |
|---|---|---|---|
| YfdW | NP_416875.1 | 16130306 | Escherichia coli |
| frc | O06644.3 | 21542067 | Oxalobacter formigenes |
| frc | ZP_04021099.1 | 227903294 | Lactobacillus acidophilus |

Suitable CoA-donor regeneration or formate transferase enzymes are encoded by the gene products of cat1, cat2, and cat3 of Clostridium kluyveri. These enzymes have been shown to exhibit succinyl-CoA, 4-hydroxybutyryl-CoA, and butyryl-CoA acetyltransferase activity, respectively (Seedorf et al., Proc. Natl. Acad. Sci. USA 105:2128-2133 (2008); Sohling and Gottschalk, J Bacteriol 178:871-880 (1996)). Similar CoA transferase activities are also present in Trichomonas vaginalis (van Grinsven et al., J. Biol. Chem. 283:1411-1418 (2008)) and Trypanosoma brucei (Riviere et al., J Biol. Chem. 279:45337-45346 (2004)). Yet another transferase capable of the desired conversions is butyryl-CoA:acetoacetate CoA-transferase. Exemplary enzymes can be found in Fusobacterium nucleatum (Barker et al., J Bacteriol. 152(1):201-7 (1982)), Clostridium SB4 (Barker et al., J. Biol. Chem. 253(4):1219-25 (1978)), and Clostridium acetobutylicum (Wiesenborn et al., Appl. Environ. Microbiol. 55(2):323-9 (1989)). Although specific gene sequences were not provided for butyryl-CoA:acetoacetate CoA-transferase in these references, the genes FN0272 and FN0273 have been annotated as a butyrate-acetoacetate CoA-transferase (Kapatral et al., J. Bact. 184(7) 2005-2018 (2002)). Homologs in Fusobacterium nucleatum such as FN1857 and FN1856 also likely have the desired acetoacetyl-CoA transferase activity. FN1857 and FN1856 are located adjacent to many other genes involved in lysine fermentation and are thus very likely to encode an acetoacetate:butyrate CoA transferase (Kreimeyer, et al., J. Biol. Chem. 282 (10) 7191-7197 (2007)). Additional candidates from Porphyrmonas gingivalis and Thermoanaerobacter tengcongensis can be identified in a similar fashion (Kreimeyer, et al., J. Biol. Chem. 282 (10) 7191-7197 (2007)). Information related to these proteins and genes is shown below.

| Protein | GenBank ID | GI number | Organism |
|---|---|---|---|
| Cat1 | P38946.1 | 729048 | Clostridium kluyveri |
| Cat2 | P38942.2 | 1705614 | Clostridium kluyveri |
| Cat3 | EDK35586.1 | 146349050 | Clostridium kluyveri |
| TVAG_395550 | XP_001330176 | 123975034 | Trichomonas vaginalis G3 |
| Tb11.02.0290 | XP_828352 | 71754875 | Trypanosoma brucei |
| FN0272 | NP_603179.1 | 19703617 | Fusobacterium nucleatum |
| FN0273 | NP_603180.1 | 19703618 | Fusobacterium nucleatum |
| FN1857 | NP_602657.1 | 19705162 | Fusobacterium nucleatum |
| FN1856 | NP_602656.1 | 19705161 | Fusobacterium nucleatum |
| PG1066 | NP_905281.1 | 34540802 | Porphyromonas gingivalis |
| | | | W83 |
| PG1075 | NP_905290.1 | 34540811 | Porphyromonas gingivalis |
| | | | W83 |
| TTE0720 | NP_622378.1 | 20807207 | Thermoanaerobacter |
| | | | tengcongensis MB4 |
| TTE0721 | NP_622379.1 | 20807208 | Thermoanaerobacter |
| | | | tengcongensis MB4 |

Additional transferase enzymes of interest include the gene products of atoAD from E. coli (Hanai et al., Appl Environ Microbiol 73:7814-7818 (2007)), ctfAB from C. acetobutylicum (Jojima et al., Appl Microbiol Biotechnol 77:1219-1224 (2008)), and ctfAB from Clostridium saccharoperbutylacetonicum (Kosaka et al., Biosci. Biotechnol Biochem. 71:58-68 (2007)). Information related to these proteins and genes is shown below.

| Protein | GenBank ID | GI number | Organism |
|---|---|---|---|
| AtoA | P76459.1 | 2492994 | Escherichia coli |
| AtoD | P76458.1 | 2492990 | Escherichia coli |
| CtfA | NP_149326.1 | 15004866 | Clostridium acetobutylicum |
| CtfB | NP_149327.1 | 15004867 | Clostridium acetobutylicum |
| CtfA | AAP42564.1 | 31075384 | Clostridium |
| | | | saccharoperbutylacetonicum |
| CtfB | AAP42565.1 | 31075385 | Clostridium |
| | | | saccharoperbutylacetonicum |

Succinyl-CoA:3-ketoacid-CoA transferase naturally converts succinate to succinyl-CoA while converting a 3-ketoacyl-CoA to a 3-ketoacid. Exemplary succinyl-CoA:3:ketoacid-CoA transferases are present in Helicobacter pylori (Corthesy-Theulaz et al., J. Biol. Chem. 272:25659-25667 (1997)), Bacillus subtilis (Stols et al., Protein. Expr. Purif. 53:396-403 (2007)), and Homo sapiens (Fukao et al., Genomics 68:144-151 (2000); Tanaka et al., Mol. Hum. Reprod. 8:16-23 (2002)). Information related to these proteins and genes is shown below.

| Protein | GenBank ID | GI number | Organism |
|---|---|---|---|
| HPAG1_0676 | YP_627417 | 108563101 | Helicobacter pylori |
| HPAG1_0677 | YP_627418 | 108563102 | Helicobacter pylori |
| ScoA | NP_391778 | 16080950 | Bacillus subtilis |
| ScoB | NP_391777 | 16080949 | Bacillus subtilis |
| OXCT1 | NP_000427 | 4557817 | Homo sapiens |
| OXCT2 | NP_071403 | 11545841 | Homo sapiens |

Two additional enzymes that catalyze the activation of formate to formyl-CoA reaction are AMP-forming formyl-CoA synthetase and ADP-forming formyl-CoA synthetase. Exemplary enzymes, known to function on acetate, are found in E. coli (Brown et al., J Gen. Microbiol. 102:327-336 (1977)), Ralstonia eutropha (Priefert and Steinbuchel, J. Bacteriol. 174:6590-6599 (1992)), Methanothermobacter thermautotrophicus (Ingram-Smith and Smith, Archaea 2:95-107 (2007)), Salmonella enterica (Gulick et al., Biochemistry 42:2866-2873 (2003)) and Saccharomyces cerevisiae (Jogl and Tong, Biochemistry 43:1425-1431 (2004)). Such enzymes may also acylate formate naturally or can be engineered to do so.

| Protein | GenBank ID | GI Number | Organism |
|---|---|---|---|
| acs | AAC77039.1 | 1790505 | Escherichia coli |
| acoE | AAA21945.1 | 141890 | Ralstonia eutropha |
| acs1 | ABC87079.1 | 86169671 | Methanothermobacter |
| | | | thermautotrophicus |
| acs1 | AAL23099.1 | 16422835 | Salmonella enterica |
| ACS1 | Q01574.2 | 257050994 | Saccharomyces cerevisiae |

ADP-forming acetyl-CoA synthetase (ACD, EC 6.2.1.13) is another candidate enzyme that couples the conversion of acyl-CoA esters to their corresponding acids with the concurrent synthesis of ATP. Several enzymes with broad substrate specificities have been described in the literature. ACD I from Archaeoglobus fulgidus, encoded by AF 1211, was shown to operate on a variety of linear and branched-chain substrates including acetyl-CoA, propionyl-CoA, butyryl-CoA, acetate, propionate, butyrate, isobutyryate, isovalerate, succinate, fumarate, phenylacetate, indoleacetate (Musfeldt et al., J. Bacteriol. 184:636-644 (2002)). The enzyme from Haloarcula marismortui (annotated as a succinyl-CoA synthetase) accepts propionate, butyrate, and branched-chain acids (isovalerate and isobutyrate) as substrates, and was shown to operate in the forward and reverse directions (Brasen et al., Arch. Microbiol. 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen et al., supra (2004)). The enzymes from A. fulgidus, H marismortui and P. aerophilum have all been cloned, functionally expressed, and characterized in E. coli (Musfeldt et al., supra; Brasen et al., supra (2004)). Additional candidates include the succinyl-CoA synthetase encoded by sucCD in E. coli (Buck et al., Biochemistry 24:6245-6252 (1985)) and the acyl-CoA ligase from Pseudomonas putida (Fernandez-Valverde et al., Appl. Environ. Microbiol. 59:1149-1154 (1993)). Such enzymes may also acylate formate naturally or can be engineered to do so. Information related to these proteins and genes is shown below.

| Protein | GenBank ID | GI number | Organism |
|---|---|---|---|
| AF1211 | NP_070039.1 | 11498810 | Archaeoglobus fulgidus DSM4304 |
| AF1983 | NP_070807.1 | 11499565 | Archaeoglobus fulgidus DSM4304 |
| scs | YP_135572.1 | 55377722 | Haloarcula marismortui ATCC |
| | | | 43049 |
| PAE3250 | NP_560604.1 | 18313937 | Pyrobaculum aerophilum str. IM2 |
| sucC | NP_415256.1 | 16128703 | Escherichia coli |
| sucD | AAC73823.1 | 1786949 | Escherichia coli |
| paaF | AAC24333.2 | 22711873 | Pseudomonas putida |

An alternative method for adding the CoA moiety to formate is to apply a pair of enzymes such as a phosphate-transferring acyltransferase and a kinase. These activities enable the net formation of formyl-CoA with the simultaneous consumption of ATP. An exemplary phosphate-transferring acyltransferase is phosphotransacetylase, encoded by pta. The pta gene from E. coli encodes an enzyme that can convert acetyl-CoA into acetyl-phosphate, and vice versa (Suzuki, T. Biochim. Biophys. Acta 191:559-569 (1969)). This enzyme can also utilize propionyl-CoA instead of acetyl-CoA forming propionate in the process (Hesslinger et al. Mol. Microbiol 27:477-492 (1998)). Homologs exist in several other organisms including Salmonella enterica and Chlamydomonas reinhardtii. Such enzymes may also phosphorylate formate naturally or can be engineered to do so.

| Protein | GenBank ID | GI number | Organism |
|---|---|---|---|
| Pta | NP_416800.1 | 16130232 | Escherichia coli |
| Pta | NP_461280.1 | 16765665 | Salmonella enterica subsp. |
| | | | enterica serovar Typhimurium |
| | | | str. LT2 |
| PAT2 | XP_001694504.1 | 159472743 | Chlamydomonas reinhardtii |
| PAT1 | XP_001691787.1 | 159467202 | Chlamydomonas reinhardtii |

An exemplary acetate kinase is the E. coli acetate kinase, encoded by ackA (Skarstedt and Silverstein J. Biol. Chem. 251:6775-6783 (1976)). Homologs exist in several other organisms including Salmonella enterica and Chlamydomonas reinhardtii. It is likely that such enzymes naturally possess formate kinase activity or can be engineered to have this activity. Information related to these proteins and genes is shown below:

| Protein | GenBank ID | GI number | Organism |
|---|---|---|---|
| AckA | NP_416799.1 | 16130231 | Escherichia coli |
| AckA | NP_461279.1 | 16765664 | Salmonella enterica subsp. |
| | | | enterica serovar Typhimurium |
| | | | str. LT2 |
| ACK1 | XP_001694505.1 | 159472745 | Chlamydomonas reinhardtii |
| ACK2 | XP_001691682.1 | 159466992 | Chlamydomonas reinhardtii |

The acylation of formate to formyl-CoA can also be carried out by a formate ligase. For example, the product of the LSC1 and LSC2 genes of S. cerevisiae and the sucC and sucD genes of E. coli naturally form a succinyl-CoA ligase complex that catalyzes the formation of succinyl-CoA from succinate with the concomitant consumption of one ATP, a reaction which is reversible in vivo (Gruys et al., U.S. Pat. No. 5,958,745, filed Sep. 28, 1999). Such enzymes may also acylate formate naturally or can be engineered to do so. Information related to these proteins and genes is shown below.

| Protein | GenBank ID | GI number | Organism |
|---|---|---|---|
| SucC | NP_415256.1 | 16128703 | Escherichia coli |
| SucD | AAC73823.1 | 1786949 | Escherichia coli |
| LSC1 | NP_014785 | 6324716 | Saccharomyces cerevisiae |
| LSC2 | NP_011760 | 6321683 | Saccharomyces cerevisiae |

Additional exemplary CoA-ligases include the rat dicarboxylate-CoA ligase for which the sequence is yet uncharacterized (Vamecq et al., Biochemical J. 230:683-693 (1985)), either of the two characterized phenylacetate-CoA ligases from P. chrysogenum (Lamas-Maceiras et al., Biochem. J 395:147-155 (2005); Wang et al., Biochem Biophy Res Commun 360(2):453-458 (2007)), the phenylacetate-CoA ligase from Pseudomonas putida (Martinez-Blanco et al., J. Biol. Chem. 265:7084-7090 (1990)), and the 6-carboxyhexanoate-CoA ligase from Bacillus subtilis (Bower et al., J. Bacteriol. 178(14):4122-4130 (1996)). Additional candidate enzymes are acetoacetyl-CoA synthetases from Mus musculus (Hasegawa et al., Biochim. Biophys. Acta 1779:414-419 (2008)) and Homo sapiens (Ohgami et al., Biochem. Pharmacol. 65:989-994 (2003)), which naturally catalyze the ATP-dependant conversion of acetoacetate into acetoacetyl-CoA. 4-Hydroxybutyryl-CoA synthetase activity has been demonstrated in Metallosphaera sedula (Berg et al., Science 318:1782-1786 (2007)). This function has been tentatively assigned to the Msed_{—}1422 gene. Such enzymes may also acylate formate naturally or can be engineered to do so. Information related to these proteins and genes is shown below.

| Protein | GenBank ID | GI number | Organism |
|---|---|---|---|
| Phl | CAJ15517.1 | 77019264 | Penicillium chrysogenum |
| PhlB | ABS19624.1 | 152002983 | Penicillium chrysogenum |
| PaaF | AAC24333.2 | 22711873 | Pseudomonas putida |
| BioW | NP_390902.2 | 50812281 | Bacillus subtilis |
| AACS | NP_084486.1 | 21313520 | Mus musculus |
| AACS | NP_076417.2 | 31982927 | Homo sapiens |
| Msed_1422 | YP_001191504 | 146304188 | Metallosphaera sedula |

### Step G, FIG. 1: Formyl-CoA Reductase

Several acyl-CoA dehydrogenases are capable of reducing an acyl-CoA (e.g., formyl-CoA) to its corresponding aldehyde (e.g., formaldehyde) (Steps F, FIG. 1). Exemplary genes that encode such enzymes include the Acinetobacter calcoaceticus acr1 encoding a fatty acyl-CoA reductase (Reiser and Somerville, J. Bacteriol. 179:2969-2975 (1997), the Acinetobacter sp. M-1 fatty acyl-CoA reductase (Ishige et al., Appl. Environ. Microbiol. 68:1192-1195 (2002), and a CoA- and NADP-dependent succinate semialdehyde dehydrogenase encoded by the sucD gene in Clostridium kluyveri (Sohling and Gottschalk, J. Bacteriol. 178:871-880 (1996); Sohling and Gottschalk, J Bacteriol. 1778:871-880 (1996)). SucD of P. gingivalis is another succinate semialdehyde dehydrogenase (Takahashi et al., J Bacteriol. 182:4704-4710 (2000). The enzyme acylating acetaldehyde dehydrogenase in Pseudomonas sp, encoded by bphG, is yet another candidate as it has been demonstrated to oxidize and acylate acetaldehyde, propionaldehyde, butyraldehyde, isobutyraldehyde and formaldehyde (Powlowski et al., J. Bacteriol. 175:377-385 (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Kazahaya et al., J. Gen. Appl. Microbiol. 18:45-55 (1972); Koo et al., Biotechnol. Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci. Biotechnol. Biochem. 71:58-68 (2007)). Additional aldehyde dehydrogenase enzyme candidates are found in Desulfatibacillum alkenivorans, Citrobacter koseri, Salmonella enterica, Lactobacillus brevis and Bacillus selenitireducens. Such enzymes may be capable of naturally converting formyl-CoA to formaldehyde or can be engineered to do so.

| Protein | GenBank ID | GI number | Organism |
|---|---|---|---|
| acr1 | YP_047869.1 | 50086355 | Acinetobacter calcoaceticus |
| acr1 | AAC45217 | 1684886 | Acinetobacter baylyi |
| acr1 | BAB85476.1 | 18857901 | Acinetobacter sp. Strain M-1 |
| sucD | P38947.1 | 172046062 | Clostridium kluyveri |
| sucD | NP_904963.1 | 34540484 | Porphyromonas gingivalis |
| bphG | BAA03892.1 | 425213 | Pseudomonas sp |
| adhE | AAV66076.1 | 55818563 | Leuconostoc mesenteroides |
| Bld | AAP42563.1 | 31075383 | Clostridium saccharoperbutylacetonicum |
| Ald | ACL06658.1 | 218764192 | Desulfatibacillum alkenivorans AK-01 |
| Ald | YP_001452373 | 157145054 | Citrobacter koseri ATCC BAA-895 |
| pduP | NP_460996.1 | 16765381 | Salmonella enterica Typhimurium |
| pduP | ABJ64680.1 | 116099531 | Lactobacillus brevis ATCC 367 |
| BselDRAFT_1651 | ZP_02169447 | 163762382 | Bacillus selenitireducens MLS10 |

An additional enzyme type that converts an acyl-CoA to its corresponding aldehyde is malonyl-CoA reductase which transforms malonyl-CoA to malonic semialdehyde. Malonyl-CoA reductase is a key enzyme in autotrophic carbon fixation via the 3-hydroxypropionate cycle in thermoacidophilic archaeal bacteria (Berg et al., Science 318:1782-1786 (2007); Thauer, Science 318:1732-1733 (2007)). The enzyme utilizes NADPH as a cofactor and has been characterized in Metallosphaera and Sulfolobus spp (Alber et al., J. Bacteriol. 188:8551-8559 (2006); Bugler et al., J. Bacteriol. 184:2404-2410 (2002)). The enzyme is encoded by Msed_{—}0709 in Metallosphaera sedula (Alber et al., supra (2006); Berg et al., Science 318:1782-1786 (2007)). A gene encoding a malonyl-CoA reductase from Sulfolobus tokodaii was cloned and heterologously expressed in E. coli (Alber et al., J. Bacteriol. 188:8551-8559 (2006)). This enzyme has also been shown to catalyze the conversion of methylmalonyl-CoA to its corresponding aldehyde (WO 2007/141208 (2007)). Although the aldehyde dehydrogenase functionality of these enzymes is similar to the bifunctional dehydrogenase from Chlorojlexus aurantiacus, there is little sequence similarity. Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates can be found by sequence homology to proteins in other organisms including Sulfolobus solfataricus and Sulfolobus acidocaldarius and have been listed below. Yet another candidate for CoA-acylating aldehyde dehydrogenase is the ald gene from Clostridium beijerinckii (Toth et al., Appl. Environ. Microbiol. 65:4973-4980 (1999). This enzyme has been reported to reduce acetyl-CoA and butyryl-CoA to their corresponding aldehydes. This gene is very similar to eutE that encodes acetaldehyde dehydrogenase of Salmonella typhimurium and E. coli (Toth et al., supra). Such enzymes may be capable of naturally converting formyl-CoA to formaldehyde or can be engineered to do so.

| Protein | GenBank ID | GI number | Organism |
|---|---|---|---|
| Msed_0709 | YP_001190808.1 | 146303492 | Metallosphaera sedula |
| Mcr | NP_378167.1 | 15922498 | Sulfolobus tokodaii |
| asd-2 | NP_343563.1 | 15898958 | Sulfolobus solfataricus |
| Saci_2370 | YP_256941.1 | 70608071 | Sulfolobus acidocaldarius |
| Ald | AAT66436 | 9473535 | Clostridium beijerinckii |
| eutE | AAA80209 | 687645 | Salmonella typhimurium |
| eutE | P77445 | 2498347 | Escherichia coli |

### Step H, FIG. 1: Formyltetrahydrofolate Synthetase

Formyltetrahydrofolate synthetase ligates formate to tetrahydrofolate at the expense of one ATP. This reaction is catalyzed by the gene product of Moth_{—}0109 in M. thermoacetica (O'brien et al., Experientia Suppl. 26:249-262 (1976); Lovell et al., Arch. Microbiol. 149:280-285 (1988); Lovell et al., Biochemistry 29:5687-5694 (1990)), FHS in Clostridium acidurici (Whitehead and Rabinowitz, J Bacteriol. 167:203-209 (1986); Whitehead and Rabinowitz, J. Bacteriol. 170:3255-3261 (1988), and CHY_{—}2385 in C. hydrogenoformans (Wu et al., PLoS Genet. 1:e65 (2005). Homologs exist in C. carboxidivorans P7. This enzyme is found in several other organisms as listed below.

| Protein | GenBank ID | GI number | Organism |
|---|---|---|---|
| Moth_0109 | YP_428991.1 | 83588982 | Moorella thermoacetica |
| CHY_2385 | YP_361182.1 | 78045024 | Carboxydothermus hydrogenoformans |
| FHS | P13419.1 | 120562 | Clostridium acidurici |
| CcarbDRAFT_1913 | ZP_05391913.1 | 255524966 | Clostridium carboxidivorans P7 |
| CcarbDRAFT_2946 | ZP_05392946.1 | 255526022 | Clostridium carboxidivorans P7 |
| Dhaf_0555 | ACL18622.1 | 219536883 | Desulfitobacterium hafniense |
| fhs | YP_001393842.1 | 153953077 | Clostridium kluyveri DSM 555 |
| fhs | YP_003781893.1 | 300856909 | Clostridium ljungdahlii DSM 13528 |
| MGA3_08300 | EIJ83208.1 | 387590889 | Bacillus methanolicus MGA3 |
| PB1_13509 | ZP_10132113.1 | 387929436 | Bacillus methanolicus PBI |

### Steps I and J, FIG. 1: Formyltetrahydrofolate Synthetase and Methylenetetrahydrofolate Dehydrogenase

In M. thermoacetica, E. coli, and C. hydrogenoformans, methenyltetrahydrofolate cyclohydrolase and methylenetetrahydrofolate dehydrogenase are carried out by the bi-functional gene products of Moth_{—}1516, folD, and CHY_{—}1878, respectively (Pierce et al., Environ. Microbiol. 10:2550-2573 (2008); Wu et al., PLoS Genet. 1:e65 (2005); D'Ari and Rabinowitz, J. Biol. Chem. 266:23953-23958 (1991)). A homolog exists in C. carboxidivorans P7. Several other organisms also encode for this bifunctional protein as tabulated below.

| Protein | GenBank ID | GI number | Organism |
|---|---|---|---|
| Moth_1516 | YP_430368.1 | 83590359 | Moorella thermoacetica |
| folD | NP_415062.1 | 16128513 | Escherichia coli |
| CHY_1878 | YP_360698.1 | 78044829 | Carboxydothermus hydrogenoformans |
| CcarbDRAFT_2948 | ZP_05392948.1 | 255526024 | Clostridium carboxidivorans P7 |
| folD | ADK16789.1 | 300437022 | Clostridium ljungdahlii DSM 13528 |
| folD-2 | NP_951919.1 | 39995968 | Geobacter sulfurreducens PCA |
| folD | YP_725874.1 | 113867385 | Ralstonia eutropha H16 |
| folD | NP_348702.1 | 15895353 | Clostridium acetobutylicum ATCC 824 |
| folD | YP_696506.1 | 110800457 | Clostridium perfringens |
| MGA3_09460 | EIJ83438.1 | 387591119 | Bacillus methanolicus MGA3 |
| PB1_14689 | ZP_10132349.1 | 387929672 | Bacillus methanolicus PB1 |

### Steps K, FIG. 1: Formaldehyde-Forming Enzyme or Spontaneous

Methylene-THF, or active formaldehyde, will spontaneously decompose to formaldehyde and THF (Thorndike and Beck, Cancer Res. 1977, 37(4) 1125-32; Ordonez and Caraballo, Psychopharmacol Commun. 1975 1(3) 253-60; Kallen and Jencks, 1966, J Biol Chem 241(24) 5851-63). To achieve higher rates, a formaldehyde-forming enzyme can be applied. Such an activity can be obtained by engineering an enzyme that reversibly forms methylene-THF from THF and a formaldehyde donor, to release free formaldehyde. Such enzymes include glycine cleavage system enzymes which naturally transfer a formaldehyde group from methylene-THF to glycine (see Step L, FIG. 1 for candidate enzymes). Additional enzymes include serine hydroxymethyltransferase (see Step M, FIG. 1 for candidate enzymes), dimethylglycine dehydrogenase (Porter, et al., Arch Biochem Biophys. 1985, 243(2) 396-407; Brizio et al., 2004, (37) 2, 434-442), sarcosine dehydrogenase (Porter, et al., Arch Biochem Biophys. 1985, 243(2) 396-407), and dimethylglycine oxidase (Leys, et al., 2003, The EMBO Journal 22(16) 4038-4048).

| Protein | GenBank ID | GI number | Organism |
|---|---|---|---|
| dmgo | ZP_09278452.1 | 359775109 | Arthrobacter globiformis |
| dmgo | YP_002778684.1 | 226360906 | Rhodococcus opacus B4 |
| dmgo | EFY87157.1 | 322695347 | Metarhizium acridum CQMa |
| | | | 102 |
| shd | AAD53398.2 | 5902974 | Homo sapiens |
| shd | NP_446116.1 | GI:25742657 | Rattus norvegicus |
| dmgdh | NP_037523.2 | 24797151 | Homo sapiens |
| dmgdh | Q63342.1 | 2498527 | Rattus norvegicus |

### Step L, FIG. 1: Glycine Cleavage System

The reversible NAD(P)H-dependent conversion of 5,10-methylenetetrahydrofolate and CO₂ to glycine is catalyzed by the glycine cleavage complex, also called glycine cleavage system, composed of four protein components; P, H, T and L. The glycine cleavage complex is involved in glycine catabolism in organisms such as E. coli and glycine biosynthesis in eukaryotes (Kikuchi et al, Proc Jpn Acad Ser 84:246 (2008)). The glycine cleavage system of E. coli is encoded by four genes: gcvPHT and lpdA (Okamura et al, Eur J Biochem 216:539-48 (1993); Heil et al, Microbiol 148:2203-14 (2002)). Activity of the glycine cleavage system in the direction of glycine biosynthesis has been demonstrated in vivo in Saccharomyces cerevisiae (Maaheimo et al, Eur J Biochem 268:2464-79 (2001)). The yeast GCV is encoded by GCV1, GCV2, GCV3 and LPD1.

| Protein | GenBank ID | GI Number | Organism |
|---|---|---|---|
| gcvP | AAC75941.1 | 1789269 | Escherichia coli |
| gcvT | AAC75943.1 | 1789272 | Escherichia coli |
| gcvH | AAC75942.1 | 1789271 | Escherichia coli |
| lpdA | AAC73227.1 | 1786307 | Escherichia coli |
| GCV1 | NP_010302.1 | 6320222 | Saccharomyces cerevisiae |
| GCV2 | NP_013914.1 | 6323843 | Saccharomyces cerevisiae |
| GCV3 | NP_009355.3 | 269970294 | Saccharomyces cerevisiae |
| LPD1 | NP_116635.1 | 14318501 | Saccharomyces cerevisiae |

### Step M, FIG. 1: Serine Hydroxymethyltransferase

Conversion of glycine to serine is catalyzed by serine hydroxymethyltransferase, also called glycine hydroxymethyltranferase. This enzyme reversibly converts glycine and 5,10-methylenetetrahydrofolate to serine and THF. Serine methyltransferase has several side reactions including the reversible cleavage of 3-hydroxyacids to glycine and an aldehyde, and the hydrolysis of 5,10-methenyl-THF to 5-formyl-THF. This enzyme is encoded by glyA of E. coli (Plamann et al, Gene 22:9-18 (1983)). Serine hydroxymethyltranferase enzymes of S. cerevisiae include SHM1 (mitochondrial) and SHM2 (cytosolic) (McNeil et al, J Biol Chem 269:9155-65 (1994)). Similar enzymes have been studied in Corynebacterium glutamicum and Methylobacterium extorquens (Chistoserdova et al, J Bacteriol 176:6759-62 (1994); Schweitzer et al, J Biotechnol 139:214-21 (2009)).

| Protein | GenBank ID | GI Number | Organism |
|---|---|---|---|
| glyA | AAC75604.1 | 1788902 | Escherichia coli |
| SHM1 | NP_009822.2 | 37362622 | Saccharomyces cerevisiae |
| SHM2 | NP_013159.1 | 6323087 | Saccharomyces cerevisiae |
| glyA | AAA64456.1 | 496116 | Methylobacterium extorquens |
| glyA | AAK60516.1 | 14334055 | Corynebacterium glutamicum |

### Step N, FIG. 1: Serine Deaminase

Serine can be deaminated to pyruvate by serine deaminase. Serine deaminase enzymes are present in several organisms including Clostridium acidurici (Carter, et al., 1972, J Bacteriol., 109(2) 757-763), Escherichia coli (Cicchillo et al., 2004, J Biol Chem., 279(31) 32418-25), and Corneybacterium sp. (Netzer et al., Appl Environ Microbiol. 2004 December; 70(12):7148-55).

| Protein | GenBank ID | GI Number | Organism |
|---|---|---|---|
| sdaA | YP_490075.1 | 388477887 | Escherichia coli |
| sdaB | YP_491005.1 | 388478813 | Escherichia coli |
| tdcG | YP_491301.1 | 388479109 | Escherichia coli |
| tdcB | YP_491307.1 | 388479115 | Escherichia coli |
| sdaA | YP_225930.1 | 62390528 | Corynebacterium sp. |

### Step O, FIG. 1: Methylenetetrahydrofolate Reductase

In M. thermoacetica, this enzyme is oxygen-sensitive and contains an iron-sulfur cluster (Clark and Ljungdahl, J. Biol. Chem. 259:10845-10849 (1984). This enzyme is encoded by metF in E. coli (Sheppard et al., J Bacteriol. 181:718-725 (1999) and CHY_{—}1233 in C. hydrogenoformans (Wu et al., PLoS Genet. 1:e65 (2005). The M. thermoacetica genes, and its C. hydrogenoformans counterpart, are located near the CODH/ACS gene cluster, separated by putative hydrogenase and heterodisulfide reductase genes. Some additional gene candidates found bioinformatically are listed below. In Acetobacterium woodii metF is coupled to the Rnf complex through RnfC2 (Poehlein et al, PLoS One. 7:e33439). Homologs of RnfC are found in other organisms by blast search. The Rnf complex is known to be a reversible complex (Fuchs (2011) Annu. Rev. Microbiol. 65:631-658).

| Protein | GenBank ID | GI number | Organism |
|---|---|---|---|
| Moth_1191 | YP_430048.1 | 83590039 | Moorella thermoacetica |
| Moth_1192 | YP_430049.1 | 83590040 | Moorella thermoacetica |
| metF | NP_418376.1 | 16131779 | Escherichia coli |
| CHY_1233 | YP_360071.1 | 78044792 | Carboxydothermus hydrogenoformans |
| CLJU_c37610 | YP_003781889.1 | 300856905 | Clostridium ljungdahlii DSM 13528 |
| DesfrDRAFT_3717 | ZP_07335241.1 | 303248996 | Desulfovibrio fructosovorans JJ |
| CcarbDRAFT_2950 | ZP_05392950.1 | 255526026 | Clostridium carboxidivorans P7 |
| Ccel74_010100023124 | ZP_07633513.1 | 307691067 | Clostridium cellulovorans 743B |
| Cphy_3110 | YP_001560205.1 | 160881237 | Clostridium phytofermentans ISDg |

### Step P, FIG. 1: Acetyl-CoA Synthase

Acetyl-CoA synthase is the central enzyme of the carbonyl branch of the Wood-Ljungdahl pathway. It catalyzes the synthesis of acetyl-CoA from carbon monoxide, coenzyme A, and the methyl group from a methylated corrinoid-iron-sulfur protein. The corrinoid-iron-sulfur-protein is methylated by methyltetrahydrofolate via a methyltransferase. Expression in a foreign host entails introducing one or more of the following proteins and their corresponding activities: Methyltetrahydrofolate:corrinoid protein methyltransferase (AcsE), Corrinoid iron-sulfur protein (AcsD), Nickel-protein assembly protein (AcsF), Ferredoxin (Orf7), Acetyl-CoA synthase (AcsB and AcsC), Carbon monoxide dehydrogenase (AcsA), and Nickel-protein assembly protein (CooC).

The genes used for carbon-monoxide dehydrogenase/acetyl-CoA synthase activity typically reside in a limited region of the native genome that can be an extended operon (Ragsdale, S. W., Crit. Rev. Biochem. Mol. Biol. 39:165-195 (2004); Morton et al., J. Biol. Chem. 266:23824-23828 (1991); Roberts et al., Proc. Natl. Acad. Sci. U.S.A. 86:32-36 (1989). Each of the genes in this operon from the acetogen, M. thermoacetica, has already been cloned and expressed actively in E. coli (Morton et al. supra; Roberts et al. supra; Lu et al., J Biol. Chem. 268:5605-5614 (1993). The protein sequences of these genes can be identified by the following GenBank accession numbers.

| Protein | GenBank ID | GI number | Organism |
|---|---|---|---|
| AcsE | YP_430054 | 83590045 | Moorella thermoacetica |
| AcsD | YP_430055 | 83590046 | Moorella thermoacetica |
| AcsF | YP_430056 | 83590047 | Moorella thermoacetica |
| Orf7 | YP_430057 | 83590048 | Moorella thermoacetica |
| AcsC | YP_430058 | 83590049 | Moorella thermoacetica |
| AcsB | YP_430059 | 83590050 | Moorella thermoacetica |
| AcsA | YP_430060 | 83590051 | Moorella thermoacetica |
| CooC | YP_430061 | 83590052 | Moorella thermoacetica |

The hydrogenic bacterium, Carboxydothermus hydrogenoformans, can utilize carbon monoxide as a growth substrate by means of acetyl-CoA synthase (Wu et al., PLoS Genet. 1:e65 (2005)). In strain Z-2901, the acetyl-CoA synthase enzyme complex lacks carbon monoxide dehydrogenase due to a frameshift mutation (Wu et al. supra (2005)), whereas in strain DSM 6008, a functional unframeshifted full-length version of this protein has been purified (Svetlitchnyi et al., Proc. Natl. Acad. Sci. U.S.A. 101:446-451 (2004)). The protein sequences of the C. hydrogenoformans genes from strain Z-2901 can be identified by the following GenBank accession numbers.

| Protein | GenBank ID | GI number | Organism |
|---|---|---|---|
| AcsE | YP_360065 | 78044202 | Carboxydothermus hydrogenoformans |
| AcsD | YP_360064 | 78042962 | Carboxydothermus hydrogenoformans |
| AcsF | YP_360063 | 78044060 | Carboxydothermus hydrogenoformans |
| Orf7 | YP_360062 | 78044449 | Carboxydothermus hydrogenoformans |
| AcsC | YP_360061 | 78043584 | Carboxydothermus hydrogenoformans |
| AcsB | YP_360060 | 78042742 | Carboxydothermus hydrogenoformans |
| CooC | YP_360059 | 78044249 | Carboxydothermus hydrogenoformans |

Homologous ACS/CODH genes can also be found in the draft genome assembly of Clostridium carboxidivorans P7.

| Protein | GenBank ID | GI Number | Organism |
|---|---|---|---|
| AcsA | ZP_05392944.1 | 255526020 | Clostridium carboxidivorans P7 |
| CooC | ZP_05392945.1 | 255526021 | Clostridium carboxidivorans P7 |
| AcsF | ZP_05392952.1 | 255526028 | Clostridium carboxidivorans P7 |
| AcsD | ZP_05392953.1 | 255526029 | Clostridium carboxidivorans P7 |
| AcsC | ZP_05392954.1 | 255526030 | Clostridium carboxidivorans P7 |
| AcsE | ZP_05392955.1 | 255526031 | Clostridium carboxidivorans P7 |
| AcsB | ZP_05392956.1 | 255526032 | Clostridium carboxidivorans P7 |
| Orf7 | ZP_05392958.1 | 255526034 | Clostridium carboxidivorans P7 |

The methanogenic archaeon, Methanosarcina acetivorans, can also grow on carbon monoxide, exhibits acetyl-CoA synthase/carbon monoxide dehydrogenase activity, and produces both acetate and formate (Lessner et al., Proc. Natl. Acad. Sci. U.S.A. 103:17921-17926 (2006)). This organism contains two sets of genes that encode ACS/CODH activity (Rother and Metcalf, Proc. Natl. Acad. Sci. U.S.A. 101:16929-16934 (2004)). The protein sequences of both sets of M. acetivorans genes are identified by the following GenBank accession numbers.

| Protein | GenBank ID | GI number | Organism |
|---|---|---|---|
| AcsC | NP_618736 | 20092661 | Methanosarcina acetivorans |
| AcsD | NP_618735 | 20092660 | Methanosarcina acetivorans |
| AcsF, CooC | NP_618734 | 20092659 | Methanosarcina acetivorans |
| AcsB | NP_618733 | 20092658 | Methanosarcina acetivorans |
| AcsEps | NP_618732 | 20092657 | Methanosarcina acetivorans |
| AcsA | NP_618731 | 20092656 | Methanosarcina acetivorans |
| AcsC | NP_615961 | 20089886 | Methanosarcina acetivorans |
| AcsD | NP_615962 | 20089887 | Methanosarcina acetivorans |
| AcsF, CooC | NP_615963 | 20089888 | Methanosarcina acetivorans |
| AcsB | NP_615964 | 20089889 | Methanosarcina acetivorans |
| AcsEps | NP_615965 | 20089890 | Methanosarcina acetivorans |
| AcsA | NP_615966 | 20089891 | Methanosarcina acetivorans |

The AcsC, AcsD, AcsB, AcsEps, and AcsA proteins are commonly referred to as the gamma, delta, beta, epsilon, and alpha subunits of the methanogenic CODH/ACS. Homologs to the epsilon encoding genes are not present in acetogens such as M. thermoacetica or hydrogenogenic bacteria such as C. hydrogenoformans. Hypotheses for the existence of two active CODH/ACS operons in M. acetivorans include catalytic properties (i.e., Kₘ, Vₘₐₓ, k_{cat}) that favor carboxidotrophic or aceticlastic growth or differential gene regulation enabling various stimuli to induce CODH/ACS expression (Rother et al., Arch. Microbiol. 188:463-472 (2007)).

### Step Q, FIG. 1: Pyruvate Formate Lyase

Pyruvate formate-lyase (PFL, EC 2.3.1.54), encoded by pflB in E. coli, can convert pyruvate into acetyl-CoA and formate. The activity of PFL can be enhanced by an activating enzyme encoded by pflA (Knappe et al., Proc. Natl. Acad. Sci USA 81:1332-1335 (1984); Wong et al., Biochemistry 32:14102-14110 (1993)). Keto-acid formate-lyase (EC 2.3.1.-), also known as 2-ketobutyrate formate-lyase (KFL) and pyruvate formate-lyase 4, is the gene product of tdcE in E. coli. This enzyme catalyzes the conversion of 2-ketobutyrate to propionyl-CoA and formate during anaerobic threonine degradation, and can also substitute for pyruvate formate-lyase in anaerobic catabolism (Simanshu et al., J Biosci. 32:1195-1206 (2007)). The enzyme is oxygen-sensitive and, like PflB, can require post-translational modification by PFL-AE to activate a glycyl radical in the active site (Hesslinger et al., Mol. Microbiol 27:477-492 (1998)). A pyruvate formate-lyase from Archaeglubus fulgidus encoded by pflD has been cloned, expressed in E. coli and characterized (Lehtio et al., Protein Eng Des Sel 17:545-552 (2004)). The crystal structures of the A. fulgidus and E. coli enzymes have been resolved (Lehtio et al., J Mol. Biol. 357:221-235 (2006); Leppanen et al., Structure. 7:733-744 (1999)). Additional PFL and PFL-AE candidates are found in Lactococcus lactis (Melchiorsen et al., Appl Microbiol Biotechnol 58:338-344 (2002)), and Streptococcus mutans (Takahashi-Abbe et al., Oral. Microbiol Immunol. 18:293-297 (2003)), Chlamydomonas reinhardtii (Hemschemeier et al., Eukaryot. Cell 7:518-526 (2008b); Atteia et al., J. Biol. Chem. 281:9909-9918 (2006)) and Clostridium pasteurianum (Weidner et al., J Bacteriol. 178:2440-2444 (1996)).

| Protein | GenBank ID | GI Number | Organism |
|---|---|---|---|
| pflB | NP_415423 | 16128870 | Escherichia coli |
| PflA | NP_415422.1 | 16128869 | Escherichia coli |
| tdcE | AAT48170.1 | 48994926 | Escherichia coli |
| pflD | NP_070278.1 | 11499044 | Archaeglubus fulgidus |
| Pfl | CAA03993 | 2407931 | Lactococcus lactis |
| Pfl | BAA09085 | 1129082 | Streptococcus mutans |
| PFL1 | XP_001689719.1 | 159462978 | Chlamydomonas reinhardtii |
| PflA1 | XP_001700657.1 | 159485246 | Chlamydomonas reinhardtii |
| Pfl | Q46266.1 | 2500058 | Clostridium pasteurianum |
| Act | CAA63749.1 | 1072362 | Clostridium pasteurianum |

### Step R, FIG. 1: Pyruvate Dehydrogenase, Pyruvate Ferredoxin Oxidoreductase, Pyruvate:NADP+ Oxidoreductase

The pyruvate dehydrogenase (PDH) complex catalyzes the conversion of pyruvate to acetyl-CoA (FIG. 2H). The E. coli PDH complex is encoded by the genes aceEF and lpdA. Enzyme engineering efforts have improved the E. coli PDH enzyme activity under anaerobic conditions (Kim et al., J. Bacteriol. 190:3851-3858 (2008); Kim et al., Appl. Environ. Microbiol. 73:1766-1771 (2007); Zhou et al., Biotechnol. Lett. 30:335-342 (2008)). In contrast to the E. coli PDH, the B. subtilis complex is active and required for growth under anaerobic conditions (Nakano et al., 179:6749-6755 (1997)). The Klebsiella pneumoniae PDH, characterized during growth on glycerol, is also active under anaerobic conditions (Menzel et al., 56:135-142 (1997)). Crystal structures of the enzyme complex from bovine kidney (Zhou et al., 98:14802-14807 (2001)) and the E2 catalytic domain from Azotobacter vinelandii are available (Mattevi et al., Science. 255:1544-1550 (1992)). Some mammalian PDH enzymes complexes can react on alternate substrates such as 2-oxobutanoate. Comparative kinetics of Rattus norvegicus PDH and BCKAD indicate that BCKAD has higher activity on 2-oxobutanoate as a substrate (Paxton et al., Biochem. J. 234:295-303 (1986)). The S. cerevisiae PDH complex can consist of an E2 (LAT1) core that binds E1 (PDA1, PDB1), E3 (LPD1), and Protein X (PDX1) components (Pronk et al., Yeast 12:1607-1633 (1996)). The PDH complex of S. cerevisiae is regulated by phosphorylation of E1 involving PKP1 (PDH kinase I), PTC5 (PDH phosphatase I), PKP2 and PTC6. Modification of these regulators may also enhance PDH activity. Coexpression of lipoyl ligase (LplA of E. coli and AIM22 in S. cerevisiae) with PDH in the cytosol may be necessary for activating the PDH enzyme complex. Increasing the supply of cytosolic lipoate, either by modifying a metabolic pathway or media supplementation with lipoate, may also improve PDH activity.

| Gene | Accession No. | GI Number | Organism |
|---|---|---|---|
| aceE | NP_414656.1 | 16128107 | Escherichia coli |
| aceF | NP_414657.1 | 16128108 | Escherichia coli |
| lpd | NP_414658.1 | 16128109 | Escherichia coli |
| lplA | NP_418803.1 | 16132203 | Escherichia coli |
| pdhA | P21881.1 | 3123238 | Bacillus subtilis |
| pdhB | P21882.1 | 129068 | Bacillus subtilis |
| pdhC | P21883.2 | 129054 | Bacillus subtilis |
| pdhD | P21880.1 | 118672 | Bacillus subtilis |
| aceE | YP_001333808.1 | 152968699 | Klebsiella pneumoniae |
| aceF | YP_001333809.1 | 152968700 | Klebsiella pneumoniae |
| lpdA | YP_001333810.1 | 152968701 | Klebsiella pneumoniae |
| Pdha1 | NP_001004072.2 | 124430510 | Rattus norvegicus |
| Pdha2 | NP_446446.1 | 16758900 | Rattus norvegicus |
| Dlat | NP_112287.1 | 78365255 | Rattus norvegicus |
| Dld | NP_955417.1 | 40786469 | Rattus norvegicus |
| LAT1 | NP_014328 | 6324258 | Saccharomyces cerevisiae |
| PDA1 | NP_011105 | 37362644 | Saccharomyces cerevisiae |
| PDB1 | NP_009780 | 6319698 | Saccharomyces cerevisiae |
| LPD1 | NP_116635 | 14318501 | Saccharomyces cerevisiae |
| PDX1 | NP_011709 | 6321632 | Saccharomyces cerevisiae |
| AIM22 | NP_012489.2 | 83578101 | Saccharomyces cerevisiae |

As an alternative to the large multienzyme PDH complexes described above, some organisms utilize enzymes in the 2-ketoacid oxidoreductase family (OFOR) to catalyze acylating oxidative decarboxylation of 2-ketoacids. Unlike the PDH complexes, PFOR enzymes contain iron-sulfur clusters, utilize different cofactors and use ferredoxin or flavodixin as electron acceptors in lieu of NAD(P)H. Pyruvate ferredoxin oxidoreductase (PFOR) can catalyze the oxidation of pyruvate to form acetyl-CoA (FIG. 2H). The PFOR from Desulfovibrio africanus has been cloned and expressed in E. coli resulting in an active recombinant enzyme that was stable for several days in the presence of oxygen (Pieulle et al., J Bacteriol. 179:5684-5692 (1997)). Oxygen stability is relatively uncommon in PFORs and is believed to be conferred by a 60 residue extension in the polypeptide chain of the D. africanus enzyme. The M. thermoacetica PFOR is also well characterized (Menon et al., Biochemistry 36:8484-8494 (1997)) and was even shown to have high activity in the direction of pyruvate synthesis during autotrophic growth (Furdui et al., J Biol Chem. 275:28494-28499 (2000)). Further, E. coli possesses an uncharacterized open reading frame, ydbK, that encodes a protein that is 51% identical to the M. thermoacetica PFOR. Evidence for pyruvate oxidoreductase activity in E. coli has been described (Blaschkowski et al., Eur. J Biochem. 123:563-569 (1982)). Several additional PFOR enzymes are described in Ragsdale, Chem. Rev. 103:2333-2346 (2003). Finally, flavodoxin reductases (e.g., fqrB from Helicobacter pylori or Campylobacter jejuni (St Maurice et al., J. Bacteriol. 189:4764-4773 (2007))) or Rnf-type proteins (Seedorf et al., Proc. Natl. Acad. Sci. U.S.A. 105:2128-2133 (2008); Hellmann et al., J. Bacteriol. 190:784-791 (2008)) provide a means to generate NADH or NADPH from the reduced ferredoxin generated by PFOR. These proteins are identified below.

| Protein | GenBank ID | GI Number | Organism |
|---|---|---|---|
| Por | CAA70873.1 | 1770208 | Desulfovibrio africanus |
| Por | YP_428946.1 | 83588937 | Moorella thermoacetica |
| ydbK | NP_415896.1 | 16129339 | Escherichia coli |
| fqrB | NP_207955.1 | 15645778 | Helicobacter pylori |
| fqrB | YP_001482096.1 | 157414840 | Campylobacter jejuni |
| RnfC | EDK33306.1 | 146346770 | Clostridium kluyveri |
| RnfD | EDK33307.1 | 146346771 | Clostridium kluyveri |
| RnfG | EDK33308.1 | 146346772 | Clostridium kluyveri |
| RnfE | EDK33309.1 | 146346773 | Clostridium kluyveri |
| RnfA | EDK33310.1 | 146346774 | Clostridium kluyveri |
| RnfB | EDK33311.1 | 146346775 | Clostridium kluyveri |

Pyruvate:NADP oxidoreductase (PNO) catalyzes the conversion of pyruvate to acetyl-CoA. This enzyme is encoded by a single gene and the active enzyme is a homodimer, in contrast to the multi-subunit PDH enzyme complexes described above. The enzyme from Euglena gracilis is stabilized by its cofactor, thiamin pyrophosphate (Nakazawa et al, Arch Biochem Biophys 411:183-8 (2003)). The mitochondrial targeting sequence of this enzyme should be removed for expression in the cytosol. The PNO protein of E. gracilis and other NADP-dependant pyruvate:NADP+ oxidoreductase enzymes are listed in the table below.

| Protein | GenBank ID | GI Number | Organism |
|---|---|---|---|
| PNO | Q94IN5.1 | 33112418 | Euglena gracilis |
| cgd4_690 | XP_625673.1 | 66356990 | Cryptosporidium |
| | | | parvum Iowa II |
| TPP_PFOR_PNO | XP_002765111.11 | 294867463 | Perkinsus marinus |
| | | | ATCC 50983 |

### Step S, FIG. 1: Formate Dehydrogenase

Formate dehydrogenase (FDH) catalyzes the reversible transfer of electrons from formate to an acceptor. Enzymes with FDH activity utilize various electron carriers such as, for example, NADH (EC 1.2.1.2), NADPH (EC 1.2.1.43), quinols (EC 1.1.5.6), cytochromes (EC 1.2.2.3) and hydrogenases (EC 1.1.99.33). FDH enzymes have been characterized from Moorella thermoacetica (Andreesen and Ljungdahl, J Bacteriol 116:867-873 (1973); Li et al., J Bacteriol 92:405-412 (1966); Yamamoto et al., J Biol Chem. 258:1826-1832 (1983). The loci, Moth_{—}2312 is responsible for encoding the alpha subunit of formate dehydrogenase while the beta subunit is encoded by Moth_{—}2314 (Pierce et al., Environ Microbiol (2008)). Another set of genes encoding formate dehydrogenase activity with a propensity for CO₂ reduction is encoded by Sfum_{—}2703 through Sfum_{—}2706 in Syntrophobacter fumaroxidans (de Bok et al., Eur J Biochem. 270:2476-2485 (2003)); Reda et al., PNAS 105:10654-10658 (2008)). A similar set of genes presumed to carry out the same function are encoded by CHY_{—}0731, CHY_{—}0732, and CHY_{—}0733 in C. hydrogenoformans (Wu et al., PLoS Genet 1:e65 (2005)). Formate dehydrogenases are also found many additional organisms including C. carboxidivorans P7, Bacillus methanolicus, Burkholderia stabilis, Moorella thermoacetica ATCC 39073, Candida boidinii, Candida methylica, and Saccharomyces cerevisiae S288c. The soluble formate dehydrogenase from Ralstonia eutropha reduces NAD⁺ (fdsG, -B, -A, -C, -D) (Oh and Bowien, 1998)

| Protein | GenBank ID | GI Number | Organism |
|---|---|---|---|
| Moth_2312 | YP_431142 | 148283121 | Moorella thermoacetica |
| Moth_2314 | YP_431144 | 83591135 | Moorella thermoacetica |
| Sfum_2703 | YP_846816.1 | 116750129 | Syntrophobacter fumaroxidans |
| Sfum_2704 | YP_846817.1 | 116750130 | Syntrophobacter fumaroxidans |
| Sfum_2705 | YP_846818.1 | 116750131 | Syntrophobacter fumaroxidans |
| Sfum_2706 | YP_846819.1 | 116750132 | Syntrophobacter fumaroxidans |
| CHY_0731 | YP_359585.1 | 78044572 | Carboxydothermus hydrogenoformans |
| CHY_0732 | YP_359586.1 | 78044500 | Carboxydothermus hydrogenoformans |
| CHY_0733 | YP_359587.1 | 78044647 | Carboxydothermus hydrogenoformans |
| CcarbDRAFT_0901 | ZP_05390901.1 | 255523938 | Clostridium carboxidivorans P7 |
| CcarbDRAFT_4380 | ZP_05394380.1 | 255527512 | Clostridium carboxidivorans P7 |
| fdhA, MGA3_06625 | EIJ82879.1 | 387590560 | Bacillus methanolicus MGA3 |
| fdhA, PB1_11719 | ZP_10131761.1 | 387929084 | Bacillus methanolicus PB1 |
| fdhD, MGA3_06630 | EIJ82880.1 | 387590561 | Bacillus methanolicus MGA3 |
| fdhD, PB1_11724 | ZP_10131762.1 | 387929085 | Bacillus methanolicus PB1 |
| fdh | ACF35003. | 194220249 | Burkholderia stabilis |
| FDH1 | AAC49766.1 | 2276465 | Candida boidinii |
| Fdh | CAA57036.1 | 1181204 | Candida methylica |
| FDH2 | P0CF35.1 | 294956522 | Saccharomyces cerevisiae S288c |
| FDH1 | NP_015033.1 | 6324964 | Saccharomyces cerevisiae S288c |

### Example II

### Production of Reducing Equivalents

This example describes methanol metabolic pathways and other additional enzymes generating reducing equivalents as shown in FIG. 3.

### FIG. 3, Step A—Methanol Methyltransferase

A complex of 3-methyltransferase proteins, denoted MtaA, MtaB, and MtaC, perform the desired methanol methyltransferase activity (Sauer et al., Eur. J. Biochem. 243:670-677 (1997); Naidu and Ragsdale, J. Bacteriol. 183:3276-3281 (2001); Tallant and Krzycki, J. Biol. Chem. 276:4485-4493 (2001); Tallant and Krzycki, J. Bacteriol. 179:6902-6911 (1997); Tallant and Krzycki, J. Bacteriol. 178:1295-1301 (1996); Ragsdale, S. W., Crit. Rev. Biochem. Mol. Biol. 39:165-195 (2004)).

MtaB is a zinc protein that can catalyze the transfer of a methyl group from methanol to MtaC, a corrinoid protein. Exemplary genes encoding MtaB and MtaC can be found in methanogenic archaea such as Methanosarcina barkeri (Maeder et al., J. Bacteriol. 188:7922-7931 (2006) and Methanosarcina acetivorans (Galagan et al., Genome Res. 12:532-542 (2002), as well as the acetogen, Moorella thermoacetica (Das et al., Proteins 67:167-176 (2007). In general, the MtaB and MtaC genes are adjacent to one another on the chromosome as their activities are tightly interdependent. The protein sequences of various MtaB and MtaC encoding genes in M. barkeri, M. acetivorans, and M. thermoaceticum can be identified by their following GenBank accession numbers.

| Protein | GenBank ID | GI number | Organism |
|---|---|---|---|
| MtaB1 | YP_304299 | 73668284 | Methanosarcina barkeri |
| MtaC1 | YP_304298 | 73668283 | Methanosarcina barkeri |
| MtaB2 | YP_307082 | 73671067 | Methanosarcina barkeri |
| MtaC2 | YP_307081 | 73671066 | Methanosarcina barkeri |
| MtaB3 | YP_304612 | 73668597 | Methanosarcina barkeri |
| MtaC3 | YP_304611 | 73668596 | Methanosarcina barkeri |
| MtaB1 | NP_615421 | 20089346 | Methanosarcina acetivorans |
| MtaB1 | NP_615422 | 20089347 | Methanosarcina acetivorans |
| MtaB2 | NP_619254 | 20093179 | Methanosarcina acetivorans |
| MtaC2 | NP_619253 | 20093178 | Methanosarcina acetivorans |
| MtaB3 | NP_616549 | 20090474 | Methanosarcina acetivorans |
| MtaC3 | NP_616550 | 20090475 | Methanosarcina acetivorans |
| MtaB | YP_430066 | 83590057 | Moorella thermoacetica |
| MtaC | YP_430065 | 83590056 | Moorella thermoacetica |
| MtaA | YP_430064 | 83590056 | Moorella thermoacetica |

The MtaB1 and MtaC1 genes, YP_{—}304299 and YP_{—}304298, from M. barkeri were cloned into E. coli and sequenced (Sauer et al., Eur. J. Biochem. 243:670-677 (1997)). The crystal structure of this methanol-cobalamin methyltransferase complex is also available (Hagemeier et al., Proc. Natl. Acad. Sci. U.S.A. 103:18917-18922 (2006)). The MtaB genes, YP_{—}307082 and YP_{—}304612, in M. barkeri were identified by sequence homology to YP_{—}304299. In general, homology searches are an effective means of identifying methanol methyltransferases because MtaB encoding genes show little or no similarity to methyltransferases that act on alternative substrates such as trimethylamine, dimethylamine, monomethylamine, or dimethylsulfide. The MtaC genes, YP_{—}307081 and YP_{—}304611 were identified based on their proximity to the MtaB genes and also their homology to YP_{—}304298. The three sets of MtaB and MtaC genes from M. acetivorans have been genetically, physiologically, and biochemically characterized (Pritchett and Metcalf, Mol. Microbiol. 56:1183-1194 (2005)). Mutant strains lacking two of the sets were able to grow on methanol, whereas a strain lacking all three sets of MtaB and MtaC genes sets could not grow on methanol. This suggests that each set of genes plays a role in methanol utilization. The M. thermoacetica MtaB gene was identified based on homology to the methanogenic MtaB genes and also by its adjacent chromosomal proximity to the methanol-induced corrinoid protein, MtaC, which has been crystallized (Zhou et al., Acta Crystallogr. Sect. F. Struct. Biol. Cyrst. Commun. 61:537-540 (2005) and further characterized by Northern hybridization and Western Blotting ((Das et al., Proteins 67:167-176 (2007)).

MtaA is zinc protein that catalyzes the transfer of the methyl group from MtaC to either Coenzyme M in methanogens or methyltetrahydrofolate in acetogens. MtaA can also utilize methylcobalamin as the methyl donor. Exemplary genes encoding MtaA can be found in methanogenic archaea such as Methanosarcina barkeri (Maeder et al., J. Bacteriol. 188:7922-7931 (2006) and Methanosarcina acetivorans (Galagan et al., Genome Res. 12:532-542 (2002), as well as the acetogen, Moorella thermoacetica ((Das et al., Proteins 67:167-176 (2007)). In general, MtaA proteins that catalyze the transfer of the methyl group from CH₃-MtaC are difficult to identify bioinformatically as they share similarity to other corrinoid protein methyltransferases and are not oriented adjacent to the MtaB and MtaC genes on the chromosomes. Nevertheless, a number of MtaA encoding genes have been characterized. The protein sequences of these genes in M. barkeri and M. acetivorans can be identified by the following GenBank accession numbers.

| Protein | GenBank ID | GI number | Organism |
|---|---|---|---|
| MtaA | YP_304602 | 73668587 | Methanosarcina barkeri |
| MtaA1 | NP_619241 | 20093166 | Methanosarcina acetivorans |
| MtaA2 | NP_616548 | 20090473 | Methanosarcina acetivorans |

The MtaA gene, YP_{—}304602, from M. barkeri was cloned, sequenced, and functionally overexpressed in E. coli (Harms and Thauer, Eur. J. Biochem. 235:653-659 (1996)). In M. acetivorans, MtaA1 is required for growth on methanol, whereas MtaA2 is dispensable even though methane production from methanol is reduced in MtaA2 mutants (Bose et al., J. Bacteriol. 190:4017-4026 (2008)). There are multiple additional MtaA homologs in M. barkeri and M. acetivorans that are as yet uncharacterized, but may also catalyze corrinoid protein methyltransferase activity.

Putative MtaA encoding genes in M. thermoacetica were identified by their sequence similarity to the characterized methanogenic MtaA genes. Specifically, three M. thermoacetica genes show high homology (>30% sequence identity) to YP_{—}304602 from M. barkeri. Unlike methanogenic MtaA proteins that naturally catalyze the transfer of the methyl group from CH₃-MtaC to Coenzyme M, an M. thermoacetica MtaA is likely to transfer the methyl group to methyltetrahydrofolate given the similar roles of methyltetrahydrofolate and Coenzyme M in methanogens and acetogens, respectively. The protein sequences of putative MtaA encoding genes from M. thermoacetica can be identified by the following GenBank accession numbers.

| Protein | GenBank ID | GI number | Organism |
|---|---|---|---|
| MtaA | YP_430937 | 83590928 | Moorella thermoacetica |
| MtaA | YP_431175 | 83591166 | Moorella thermoacetica |
| MtaA | YP_430935 | 83590926 | Moorella thermoacetica |
| MtaA | YP_430064 | 83590056 | Moorella thermoacetica |

### FIG. 3, Step B—Methylenetetrahydrofolate Reductase

The conversion of methyl-THF to methylenetetrahydrofolate is catalyzed by methylenetetrahydrofolate reductase. In M. thermoacetica, this enzyme is oxygen-sensitive and contains an iron-sulfur cluster (Clark and Ljungdahl, J. Biol. Chem. 259:10845-10849 (1984). This enzyme is encoded by metF in E. coli (Sheppard et al., J Bacteriol. 181:718-725 (1999) and CHY_{—}1233 in C. hydrogenoformans (Wu et al., PLoS Genet. 1:e65 (2005). The M. thermoacetica genes, and its C. hydrogenoformans counterpart, are located near the CODH/ACS gene cluster, separated by putative hydrogenase and heterodisulfide reductase genes. Some additional gene candidates found bioinformatically are listed below. In Acetobacterium woodii metF is coupled to the Rnf complex through RnfC2 (Poehlein et al, PLoS One. 7:e33439). Homologs of RnfC are found in other organisms by blast search. The Rnf complex is known to be a reversible complex (Fuchs (2011) Annu. Rev. Microbiol. 65:631-658).

| Protein | GenBank ID | GI number | Organism |
|---|---|---|---|
| Moth_1191 | YP_430048.1 | 83590039 | Moorella thermoacetica |
| Moth_1192 | YP_430049.1 | 83590040 | Moorella thermoacetica |
| metF | NP_418376.1 | 16131779 | Escherichia coli |
| CHY_1233 | YP_360071.1 | 78044792 | Carboxydothermus hydrogenoformans |
| CLJU_c37610 | YP_003781889.1 | 300856905 | Clostridium ljungdahlii DSM 13528 |
| DesfrDRAFT_3717 | ZP_07335241.1 | 303248996 | Desulfovibrio fructosovorans JJ |
| CcarbDRAFT_2950 | ZP_05392950.1 | 255526026 | Clostridium carboxidivorans P7 |
| Cce174_010100023124 | ZP_07633513.1 | 307691067 | Clostridium cellulovorans 743B |
| Cphy_3110 | YP_001560205.1 | 160881237 | Clostridium phytofermentans ISDg |

### FIG. 3, Steps C and D—Methylenetetrahydrofolate Dehydrogenase, Methenyltetrahydrofolate Cyclohydrolase

In M. thermoacetica, E. coli, and C. hydrogenoformans, methenyltetrahydrofolate cyclohydrolase and methylenetetrahydrofolate dehydrogenase are carried out by the bi-functional gene products of Moth_{—}1516, folD, and CHY_{—}1878, respectively (Pierce et al., Environ. Microbiol. 10:2550-2573 (2008); Wu et al., PLoS Genet. 1:e65 (2005); D'Ari and Rabinowitz, J. Biol. Chem. 266:23953-23958 (1991)). A homolog exists in C. carboxidivorans P7. Several other organisms also encode for this bifunctional protein as tabulated below.

| Protein | GenBank ID | GI number | Organism |
|---|---|---|---|
| Moth_1516 | YP_430368.1 | 83590359 | Moorella thermoacetica |
| folD | NP_415062.1 | 16128513 | Escherichia coli |
| CHY_1878 | YP_360698.1 | 78044829 | Carboxydothermus hydrogenoformans |
| CcarbDRAFT_2948 | ZP_05392948.1 | 255526024 | Clostridium carboxidivorans P7 |
| folD | ADK16789.1 | 300437022 | Clostridium ljungdahlii DSM 13528 |
| folD-2 | NP_951919.1 | 39995968 | Geobacter sulfurreducens PCA |
| folD | YP_725874.1 | 113867385 | Ralstonia eutropha H16 |
| folD | NP_348702.1 | 15895353 | Clostridium acetobutylicum ATCC 824 |
| folD | YP_696506.1 | 110800457 | Clostridium perfringens |

| | | | |
|---|---|---|---|
| MGA3_09460 | EIJ83438.1 | 387591119 | Bacillus methanolicus MGA3 |
| PB1_14689 | ZP_10132349.1 | 387929672 | Bacillus methanolicus PB1 |

### FIG. 3, Step E—Formyltetrahydrofolate Deformylase

This enzyme catalyzes the hydrolysis of 1O-formyltetrahydrofolate (formyl-THF) to THF and formate. In E. coli, this enzyme is encoded by purU and has been overproduced, purified, and characterized (Nagy, et al., J. Bacteriol. 3:1292-1298 (1995)). Homologs exist in Corynebacterium sp. U-96 (Suzuki, et al., Biosci. Biotechnol. Biochem. 69(5):952-956 (2005)), Corynebacterium glutamicum ATCC 14067, Salmonella enterica, and several additional organisms.

| Protein | GenBank ID | GI number | Organism |
|---|---|---|---|
| purU | AAC74314.1 | 1787483 | Escherichia coli K-12 MG1655 |
| purU | BAD97821.1 | 63002616 | Corynebacterium sp. U-96 |
| purU | EHE84645.1 | 354511740 | Corynebacterium glutamicum ATCC 14067 |
| purU | NP_460715.1 | 16765100 | Salmonella enterica subsp. enterica serovar Typhimurium str. LT2 |

### FIG. 3, Step F—Formyltetrahydrofolate Synthetase

Formyltetrahydrofolate synthetase ligates formate to tetrahydrofolate at the expense of one ATP. This reaction is catalyzed by the gene product of Moth_{—}0109 in M. thermoacetica (O'brien et al., Experientia Suppl. 26:249-262 (1976); Lovell et al., Arch. Microbiol. 149:280-285 (1988); Lovell et al., Biochemistry 29:5687-5694 (1990)), FHS in Clostridium acidurici (Whitehead and Rabinowitz, J Bacteriol. 167:203-209 (1986); Whitehead and Rabinowitz, J. Bacteriol. 170:3255-3261 (1988), and CHY_{—}2385 in C. hydrogenoformans (Wu et al., PLoS Genet. 1:e65 (2005). Homologs exist in C. carboxidivorans P7. This enzyme is found in several other organisms as listed below.

| Protein | GenBank ID | GI number | Organism |
|---|---|---|---|
| Moth_0109 | YP_428991.1 | 83588982 | Moorella thermoacetica |
| CHY_2385 | YP_361182.1 | 78045024 | Carboxydothermus hydrogenoformans |
| FHS | P13419.1 | 120562 | Clostridium acidurici |
| CcarbDRAFT_1913 | ZP_05391913.1 | 255524966 | Clostridium carboxidivorans P7 |
| CcarbDRAFT_2946 | ZP_05392946.1 | 255526022 | Clostridium carboxidivorans P7 |
| Dhaf_0555 | ACL18622.1 | 219536883 | Desulfitobacterium hafniense |
| fhs | YP_001393842.1 | 153953077 | Clostridium kluyveri DSM 555 |
| fhs | YP_003781893.1 | 300856909 | Clostridium ljungdahlii DSM 13528 |
| MGA3_08300 | EIJ83208.1 | 387590889 | Bacillus methanolicus MGA3 |
| PB1_13509 | ZP_10132113.1 | 387929436 | Bacillus methanolicus PB1 |

### FIG. 3, Step G—Formate Hydrogen Lyase

A formate hydrogen lyase enzyme can be employed to convert formate to carbon dioxide and hydrogen. An exemplary formate hydrogen lyase enzyme can be found in Escherichia coli. The E. coli formate hydrogen lyase consists of hydrogenase 3 and formate dehydrogenase-H (Maeda et al., Appl Microbiol Biotechnol 77:879-890 (2007)). It is activated by the gene product of fhlA. (Maeda et al., Appl Microbiol Biotechnol 77:879-890 (2007)). The addition of the trace elements, selenium, nickel and molybdenum, to a fermentation broth has been shown to enhance formate hydrogen lyase activity (Soini et al., Microb. Cell Fact. 7:26 (2008)). Various hydrogenase 3, formate dehydrogenase and transcriptional activator genes are shown below.

| Protein | GenBank ID | GI number | Organism |
|---|---|---|---|
| hycA | NP_417205 | 16130632 | Escherichia coli K-12 MG1655 |
| hycB | NP_417204 | 16130631 | Escherichia coli K-12 MG1655 |
| hycC | NP_417203 | 16130630 | Escherichia coli K-12 MG1655 |
| hycD | NP_417202 | 16130629 | Escherichia coli K-12 MG1655 |
| hycE | NP_417201 | 16130628 | Escherichia coli K-12 MG1655 |
| hycF | NP_417200 | 16130627 | Escherichia coli K-12 MG1655 |
| hycG | NP_417199 | 16130626 | Escherichia coli K-12 MG1655 |
| hycH | NP_417198 | 16130625 | Escherichia coli K-12 MG1655 |
| hycI | NP_417197 | 16130624 | Escherichia coli K-12 MG1655 |
| fdhF | NP_418503 | 16131905 | Escherichia coli K-12 MG1655 |
| fhlA | NP_417211 | 16130638 | Escherichia coli K-12 MG1655 |

A formate hydrogen lyase enzyme also exists in the hyperthermophilic archaeon, Thermococcus litoralis (Takacs et al., BMC. Microbiol 8:88 (2008)).

| Protein | GenBank ID | GI number | Organism |
|---|---|---|---|
| mhyC | ABW05543 | 157954626 | Thermococcus litoralis |
| mhyD | ABW05544 | 157954627 | Thermococcus litoralis |
| mhyE | ABW05545 | 157954628 | Thermococcus litoralis |
| myhF | ABW05546 | 157954629 | Thermococcus litoralis |
| myhG | ABW05547 | 157954630 | Thermococcus litoralis |
| myhH | ABW05548 | 157954631 | Thermococcus litoralis |
| fdhA | AAB94932 | 2746736 | Thermococcus litoralis |
| fdhB | AAB94931 | 157954625 | Thermococcus litoralis |

Additional formate hydrogen lyase systems have been found in Salmonella typhimurium, Klebsiella pneumoniae, Rhodospirillum rubrum, Methanobacterium formicicum (Vardar-Schara et al., Microbial Biotechnology 1:107-125 (2008)).

### FIG. 3, Step H—Hydrogenase

Hydrogenase enzymes can convert hydrogen gas to protons and transfer electrons to acceptors such as ferredoxins, NAD+, or NADP+. Ralstonia eutropha H16 uses hydrogen as an energy source with oxygen as a terminal electron acceptor. Its membrane-bound uptake [NiFe]-hydrogenase is an “O2-tolerant” hydrogenase (Cracknell, et al. Proc Nat Acad Sci, 106(49) 20681-20686 (2009)) that is periplasmically-oriented and connected to the respiratory chain via a b-type cytochrome (Schink and Schlegel, Biochim. Biophys. Acta, 567, 315-324 (1979); Bernhard et al., Eur. J. Biochem. 248, 179-186 (1997)). R. eutropha also contains an O₂-tolerant soluble hydrogenase encoded by the Hox operon which is cytoplasmic and directly reduces NAD+ at the expense of hydrogen (Schneider and Schlegel, Biochim. Biophys. Acta 452, 66-80 (1976); Burgdorf, J. Bact. 187(9) 3122-3132(2005)). Soluble hydrogenase enzymes are additionally present in several other organisms including Geobacter sulfurreducens (Coppi, Microbiology 151,1239 (2005)), Synechocystis str. PCC 6803 (Germer, J. Biol. Chem., 284(52), 36462-36472 (2009)), and Thiocapsa roseopersicina (Rakhely, Appl. Environ. Microbiol. 70(2) 722-728 (2004)). The Synechocystis enzyme is capable of generating NADPH from hydrogen. Overexpression of both the Hox operon from Synechocystis str. PCC 6803 and the accessory genes encoded by the Hyp operon from Nostoc sp. PCC 7120 led to increased hydrogenase activity compared to expression of the Hox genes alone (Germer, J Biol. Chem. 284(52), 36462-36472 (2009)).

| Protein | GenBank ID | GI Number | Organism |
|---|---|---|---|
| HoxF | NP_942727.1 | 38637753 | Ralstonia eutropha H16 |
| HoxU | NP_942728.1 | 38637754 | Ralstonia eutropha H16 |
| HoxY | NP_942729.1 | 38637755 | Ralstonia eutropha H16 |
| HoxH | NP_942730.1 | 38637756 | Ralstonia eutropha H16 |
| HoxW | NP_942731.1 | 38637757 | Ralstonia eutropha H16 |
| HoxI | NP_942732.1 | 38637758 | Ralstonia eutropha H16 |
| HoxE | NP_953767.1 | 39997816 | Geobacter sulfurreducens |
| HoxF | NP_953766.1 | 39997815 | Geobacter sulfurreducens |
| HoxU | NP_953765.1 | 39997814 | Geobacter sulfurreducens |
| HoxY | NP_953764.1 | 39997813 | Geobacter sulfurreducens |
| HoxH | NP_953763.1 | 39997812 | Geobacter sulfurreducens |
| GSU2717 | NP_953762.1 | 39997811 | Geobacter sulfurreducens |
| HoxE | NP_441418.1 | 16330690 | Synechocystis str. PCC 6803 |
| HoxF | NP_441417.1 | 16330689 | Synechocystis str. PCC 6803 |
| Unknown function | NP_441416.1 | 16330688 | Synechocystis str. PCC 6803 |
| HoxU | NP_441415.1 | 16330687 | Synechocystis str. PCC 6803 |
| HoxY | NP_441414.1 | 16330686 | Synechocystis str. PCC 6803 |
| Unknown function | NP_441413.1 | 16330685 | Synechocystis str. PCC 6803 |
| Unknown function | NP_441412.1 | 16330684 | Synechocystis str. PCC 6803 |
| HoxH | NP_441411.1 | 16330683 | Synechocystis str. PCC 6803 |
| HypF | NP_484737.1 | 17228189 | Nostoc sp. PCC 7120 |
| HypC | NP_484738.1 | 17228190 | Nostoc sp. PCC 7120 |
| HypD | NP_484739.1 | 17228191 | Nostoc sp. PCC 7120 |
| Unknown function | NP_484740.1 | 17228192 | Nostoc sp. PCC 7120 |
| HypE | NP_484741.1 | 17228193 | Nostoc sp. PCC 7120 |
| HypA | NP_484742.1 | 17228194 | Nostoc sp. PCC 7120 |
| HypB | NP_484743.1 | 17228195 | Nostoc sp. PCC 7120 |
| Hox1E | AAP50519.1 | 37787351 | Thiocapsa roseopersicina |
| Hox1F | AAP50520.1 | 37787352 | Thiocapsa roseopersicina |
| Hox1U | AAP50521.1 | 37787353 | Thiocapsa roseopersicina |
| Hox 1Y | AAP50522.1 | 37787354 | Thiocapsa roseopersicina |
| Hox1H | AAP50523.1 | 37787355 | Thiocapsa roseopersicina |

The genomes of E. coli and other enteric bacteria encode up to four hydrogenase enzymes (Sawers, G., Antonie Van Leeuwenhoek 66:57-88 (1994); Sawers et al., J Bacteriol. 164:1324-1331 (1985); Sawers and Boxer, Eur. J Biochem. 156:265-275 (1986); Sawers et al., J Bacteriol. 168:398-404 (1986)). Given the multiplicity of enzyme activities E. coli or another host organism can provide sufficient hydrogenase activity to split incoming molecular hydrogen and reduce the corresponding acceptor. Endogenous hydrogen-lyase enzymes of E. coli include hydrogenase 3, a membrane-bound enzyme complex using ferredoxin as an acceptor, and hydrogenase 4 that also uses a ferredoxin acceptor. Hydrogenase 3 and 4 are encoded by the hyc and hyf gene clusters, respectively. Hydrogenase activity in E. coli is also dependent upon the expression of the hyp genes whose corresponding proteins are involved in the assembly of the hydrogenase complexes (Jacobi et al., Arch. Microbiol 158:444-451 (1992); Rangaraj an et al., J Bacteriol. 190:1447-1458 (2008)). The M. thermoacetica and Clostridium ljungdahli hydrogenases are suitable for a host that lacks sufficient endogenous hydrogenase activity. M. thermoacetica and C. ljungdahli can grow with CO₂ as the exclusive carbon source indicating that reducing equivalents are extracted from H₂ to enable acetyl-CoA synthesis via the Wood-Ljungdahl pathway (Drake, H. L., J Bacteriol. 150:702-709 (1982); Drake and Daniel, Res Microbiol 155:869-883 (2004); Kellum and Drake, J Bacteriol. 160:466-469 (1984)). M. thermoacetica has homologs to several hyp, hyc, and hyf genes from E. coli. These protein sequences encoded for by these genes are identified by the following GenBank accession numbers. In addition, several gene clusters encoding hydrogenase functionality are present in M. thermoacetica and C. ljungdahli (see for example US 2012/0003652).

| Protein | GenBank ID | GI Number | Organism |
|---|---|---|---|
| HypA | NP_417206 | 16130633 | Escherichia coli |
| HypB | NP_417207 | 16130634 | Escherichia coli |
| HypC | NP_417208 | 16130635 | Escherichia coli |
| HypD | NP_417209 | 16130636 | Escherichia coli |
| HypE | NP_417210 | 226524740 | Escherichia coli |
| HypF | NP_417192 | 16130619 | Escherichia coli |
| HycA | NP_417205 | 16130632 | Escherichia coli |
| HycB | NP_417204 | 16130631 | Escherichia coli |
| HycC | NP_417203 | 16130630 | Escherichia coli |
| HycD | NP_417202 | 16130629 | Escherichia coli |
| HycE | NP_417201 | 16130628 | Escherichia coli |
| HycF | NP_417200 | 16130627 | Escherichia coli |
| HycG | NP_417199 | 16130626 | Escherichia coli |
| HycH | NP_417198 | 16130625 | Escherichia coli |
| HycI | NP_417197 | 16130624 | Escherichia coli |
| HyfA | NP_416976 | 90111444 | Escherichia coli |
| HyfB | NP_416977 | 16130407 | Escherichia coli |
| HyfC | NP_416978 | 90111445 | Escherichia coli |
| HyfD | NP_416979 | 16130409 | Escherichia coli |
| HyfE | NP_416980 | 16130410 | Escherichia coli |
| HyfF | NP_416981 | 16130411 | Escherichia coli |
| HyfG | NP_416982 | 16130412 | Escherichia coli |
| HyfH | NP_416983 | 16130413 | Escherichia coli |
| HyfI | NP_416984 | 16130414 | Escherichia coli |
| HyfJ | NP_416985 | 90111446 | Escherichia coli |
| HyfR | NP_416986 | 90111447 | Escherichia coli |

Proteins in M. thermoacetica whose genes are homologous to the E. coli hydrogenase genes are shown below.

| Protein | GenBank ID | GI Number | Organism |
|---|---|---|---|
| Moth_2175 | YP_431007 | 83590998 | Moorella thermoacetica |
| Moth_2176 | YP_431008 | 83590999 | Moorella thermoacetica |
| Moth_2177 | YP_431009 | 83591000 | Moorella thermoacetica |
| Moth_2178 | YP_431010 | 83591001 | Moorella thermoacetica |
| Moth_2179 | YP_431011 | 83591002 | Moorella thermoacetica |
| Moth_2180 | YP_431012 | 83591003 | Moorella thermoacetica |
| Moth_2181 | YP_431013 | 83591004 | Moorella thermoacetica |
| Moth_2182 | YP_431014 | 83591005 | Moorella thermoacetica |
| Moth_2183 | YP_431015 | 83591006 | Moorella thermoacetica |
| Moth_2184 | YP_431016 | 83591007 | Moorella thermoacetica |
| Moth_2185 | YP_431017 | 83591008 | Moorella thermoacetica |
| Moth_2186 | YP_431018 | 83591009 | Moorella thermoacetica |
| Moth_2187 | YP_431019 | 83591010 | Moorella thermoacetica |
| Moth_2188 | YP_431020 | 83591011 | Moorella thermoacetica |
| Moth_2189 | YP_431021 | 83591012 | Moorella thermoacetica |
| Moth_2190 | YP_431022 | 83591013 | Moorella thermoacetica |
| Moth_2191 | YP_431023 | 83591014 | Moorella thermoacetica |
| Moth_2192 | YP_431024 | 83591015 | Moorella thermoacetica |
| Moth_0439 | YP_429313 | 83589304 | Moorella thermoacetica |
| Moth_0440 | YP_429314 | 83589305 | Moorella thermoacetica |
| Moth_0441 | YP_429315 | 83589306 | Moorella thermoacetica |
| Moth_0442 | YP_429316 | 83589307 | Moorella thermoacetica |
| Moth_0809 | YP_429670 | 83589661 | Moorella thermoacetica |
| Moth_0810 | YP_429671 | 83589662 | Moorella thermoacetica |
| Moth_0811 | YP_429672 | 83589663 | Moorella thermoacetica |
| Moth_0812 | YP_429673 | 83589664 | Moorella thermoacetica |
| Moth_0814 | YP_429674 | 83589665 | Moorella thermoacetica |
| Moth_0815 | YP_429675 | 83589666 | Moorella thermoacetica |
| Moth_0816 | YP_429676 | 83589667 | Moorella thermoacetica |
| Moth_1193 | YP_430050 | 83590041 | Moorella thermoacetica |
| Moth_1194 | YP_430051 | 83590042 | Moorella thermoacetica |
| Moth_1195 | YP_430052 | 83590043 | Moorella thermoacetica |
| Moth_1196 | YP_430053 | 83590044 | Moorella thermoacetica |
| Moth_1717 | YP_430562 | 83590553 | Moorella thermoacetica |
| Moth_1718 | YP_430563 | 83590554 | Moorella thermoacetica |
| Moth_1719 | YP_430564 | 83590555 | Moorella thermoacetica |
| Moth_1883 | YP_430726 | 83590717 | Moorella thermoacetica |
| Moth_1884 | YP_430727 | 83590718 | Moorella thermoacetica |
| Moth_1885 | YP_430728 | 83590719 | Moorella thermoacetica |
| Moth_1886 | YP_430729 | 83590720 | Moorella thermoacetica |
| Moth_1887 | YP_430730 | 83590721 | Moorella thermoacetica |
| Moth_1888 | YP_430731 | 83590722 | Moorella thermoacetica |
| Moth_1452 | YP_430305 | 83590296 | Moorella thermoacetica |
| Moth_1453 | YP_430306 | 83590297 | Moorella thermoacetica |
| Moth_1454 | YP_430307 | 83590298 | Moorella thermoacetica |

Genes encoding hydrogenase enzymes from C. ljungdahli are shown below.

| Protein | GenBank ID | GI Number | Organism |
|---|---|---|---|
| CLJU_c20290 | ADK15091.1 | 300435324 | Clostridium ljungdahli |
| CLJU_c07030 | ADK13773.1 | 300434006 | Clostridium ljungdahli |
| CLJU_c07040 | ADK13774.1 | 300434007 | Clostridium ljungdahli |
| CLJU_c07050 | ADK13775.1 | 300434008 | Clostridium ljungdahli |
| CLJU_c07060 | ADK13776.1 | 300434009 | Clostridium ljungdahli |
| CLJU_c07070 | ADK13777.1 | 300434010 | Clostridium ljungdahli |
| CLJU_c07080 | ADK13778.1 | 300434011 | Clostridium ljungdahli |
| CLJU_c14730 | ADK14541.1 | 300434774 | Clostridium ljungdahli |
| CLJU_c14720 | ADK14540.1 | 300434773 | Clostridium ljungdahli |
| CLJU_c14710 | ADK14539.1 | 300434772 | Clostridium ljungdahli |
| CLJU_c14700 | ADK14538.1 | 300434771 | Clostridium ljungdahli |
| CLJU_c28670 | ADK15915.1 | 300436148 | Clostridium ljungdahli |
| CLJU_c28660 | ADK15914.1 | 300436147 | Clostridium ljungdahli |
| CLJU_c28650 | ADK15913.1 | 300436146 | Clostridium ljungdahli |
| CLJU_c28640 | ADK15912.1 | 300436145 | Clostridium ljungdahli |

In some cases, hydrogenase encoding genes are located adjacent to a CODH. In Rhodospirillum rubrum, the encoded CODH/hydrogenase proteins form a membrane-bound enzyme complex that has been indicated to be a site where energy, in the form of a proton gradient, is generated from the conversion of CO and H₂O to CO₂ and H₂ (Fox et al., J Bacteriol. 178:6200-6208 (1996)). The CODH-I of C. hydrogenoformans and its adjacent genes have been proposed to catalyze a similar functional role based on their similarity to the R. rubrum CODH/hydrogenase gene cluster (Wu et al., PLoS Genet. 1:e65 (2005)). The C. hydrogenoformans CODH-I was also shown to exhibit intense CO oxidation and CO₂ reduction activities when linked to an electrode (Parkin et al., J Am. Chem. Soc. 129:10328-10329 (2007)).

| Protein | GenBank ID | GI Number | Organism |
|---|---|---|---|
| CooL | AAC45118 | 1515468 | Rhodospirillum rubrum |
| CooX | AAC45119 | 1515469 | Rhodospirillum rubrum |
| CooU | AAC45120 | 1515470 | Rhodospirillum rubrum |
| CooH | AAC45121 | 1498746 | Rhodospirillum rubrum |
| CooF | AAC45122 | 1498747 | Rhodospirillum rubrum |
| CODH | AAC45123 | 1498748 | Rhodospirillum rubrum |
| (CooS) | | | |
| CooC | AAC45124 | 1498749 | Rhodospirillum rubrum |
| CooT | AAC45125 | 1498750 | Rhodospirillum rubrum |
| CooJ | AAC45126 | 1498751 | Rhodospirillum rubrum |
| CODH-I | YP_360644 | 78043418 | Carboxydothermus hydrogenoformans |
| (CooS-I) | | | |
| CooF | YP_360645 | 78044791 | Carboxydothermus hydrogenoformans |
| HypA | YP_360646 | 78044340 | Carboxydothermus hydrogenoformans |
| CooH | YP_360647 | 78043871 | Carboxydothermus hydrogenoformans |
| CooU | YP_360648 | 78044023 | Carboxydothermus hydrogenoformans |
| CooX | YP_360649 | 78043124 | Carboxydothermus hydrogenoformans |
| CooL | YP_360650 | 78043938 | Carboxydothermus hydrogenoformans |
| CooK | YP_360651 | 78044700 | Carboxydothermus hydrogenoformans |
| CooM | YP_360652 | 78043942 | Carboxydothermus hydrogenoformans |
| CooC | YP_360654.1 | 78043296 | Carboxydothermus hydrogenoformans |
| CooA-1 | YP_360655.1 | 78044021 | Carboxydothermus hydrogenoformans |

Some hydrogenase and CODH enzymes transfer electrons to ferredoxins. Ferredoxins are small acidic proteins containing one or more iron-sulfur clusters that function as intracellular electron carriers with a low reduction potential. Reduced ferredoxins donate electrons to Fe-dependent enzymes such as ferredoxin-NADP⁺ oxidoreductase, pyruvate:ferredoxin oxidoreductase (PFOR) and 2-oxoglutarate:ferredoxin oxidoreductase (OFOR). The H. thermophilus gene fdx1 encodes a [4Fe-4S]-type ferredoxin that is required for the reversible carboxylation of 2-oxoglutarate and pyruvate by OFOR and PFOR, respectively (Yamamoto et al., Extremophiles 14:79-85 (2010)). The ferredoxin associated with the Sulfolobus solfataricus 2-oxoacid:ferredoxin reductase is a monomeric dicluster [3Fe-4S][4Fe-4S] type ferredoxin (Park et al. 2006). While the gene associated with this protein has not been fully sequenced, the N-terminal domain shares 93% homology with the zfx ferredoxin from S. acidocaldarius. The E. coli genome encodes a soluble ferredoxin of unknown physiological function, fdx. Some evidence indicates that this protein can function in iron-sulfur cluster assembly (Takahashi and Nakamura, 1999). Additional ferredoxin proteins have been characterized in Helicobacter pylori (Mukhopadhyay et al. 2003) and Campylobacter jejuni (van Vliet et al. 2001). A 2Fe-2S ferredoxin from Clostridium pasteurianum has been cloned and expressed in E. coli (Fujinaga and Meyer, Biochemical and Biophysical Research Communications, 192(3): (1993)). Acetogenic bacteria such as Moorella thermoacetica, Clostridium carboxidivorans P7, Clostridium ljungdahli and Rhodospirillum rubrum are predicted to encode several ferredoxins, listed below.

| Protein | GenBank ID | GI Number | Organism |
|---|---|---|---|
| fdx1 | BAE02673.1 | 68163284 | Hydrogenobacter thermophilus |
| M11214.1 | AAA83524.1 | 144806 | Clostridium pasteurianum |
| Zfx | AAY79867.1 | 68566938 | Sulfolobus acidocalarius |
| Fdx | AAC75578.1 | 1788874 | Escherichia coli |
| hp_0277 | AAD07340.1 | 2313367 | Helicobacter pylori |
| fdxA | CAL34484.1 | 112359698 | Campylobacter jejuni |
| Moth_0061 | ABC18400.1 | 83571848 | Moorella thermoacetica |
| Moth_1200 | ABC19514.1 | 83572962 | Moorella thermoacetica |
| Moth_1888 | ABC20188.1 | 83573636 | Moorella thermoacetica |
| Moth_2112 | ABC20404.1 | 83573852 | Moorella thermoacetica |
| Moth_1037 | ABC19351.1 | 83572799 | Moorella thermoacetica |
| CcarbDRAFT_4383 | ZP_05394383.1 | 255527515 | Clostridium carboxidivorans P7 |
| CcarbDRAFT_2958 | ZP_05392958.1 | 255526034 | Clostridium carboxidivorans P7 |
| CcarbDRAFT_2281 | ZP_05392281.1 | 255525342 | Clostridium carboxidivorans P7 |
| CcarbDRAFT_5296 | ZP_05395295.1 | 255528511 | Clostridium carboxidivorans P7 |
| CcarbDRAFT_1615 | ZP_05391615.1 | 255524662 | Clostridium carboxidivorans P7 |
| CcarbDRAFT_1304 | ZP_05391304.1 | 255524347 | Clostridium carboxidivorans P7 |
| cooF | AAG29808.1 | 11095245 | Carboxydothermus hydrogenoformans |
| fdxN | CAA35699.1 | 46143 | Rhodobacter capsulatus |
| Rru_A2264 | ABC23064.1 | 83576513 | Rhodospirillum rubrum |
| Rru_A1916 | ABC22716.1 | 83576165 | Rhodospirillum rubrum |
| Rru_A2026 | ABC22826.1 | 83576275 | Rhodospirillum rubrum |
| cooF | AAC45122.1 | 1498747 | Rhodospirillum rubrum |
| fdxN | AAA26460.1 | 152605 | Rhodospirillum rubrum |
| Alvin_2884 | ADC63789.1 | 288897953 | Allochromatium vinosum DSM 180 |
| Fdx | YP_002801146.1 | 226946073 | Azotobacter vinelandii DJ |
| CKL_3790 | YP_001397146.1 | 153956381 | Clostridium kluyveri DSM 555 |
| fer1 | NP_949965.1 | 39937689 | Rhodopseudomonas palustris CGA009 |
| Fdx | CAA12251.1 | 3724172 | Thauera aromatica |
| CHY_2405 | YP_361202.1 | 78044690 | Carboxydothermus hydrogenoformans |
| Fer | YP_359966.1 | 78045103 | Carboxydothermus hydrogenoformans |
| Fer | AAC83945.1 | 1146198 | Bacillus subtilis |
| fdx1 | NP_249053.1 | 15595559 | Pseudomonas aeruginosa PA01 |
| yfhL | AP_003148.1 | 89109368 | Escherichia coli K-12 |
| CLJU_c00930 | ADK13195.1 | 300433428 | Clostridium ljungdahli |
| CLJU_c00010 | ADK13115.1 | 300433348 | Clostridium ljungdahli |
| CLJU_c01820 | ADK13272.1 | 300433505 | Clostridium ljungdahli |
| CLJU_c17980 | ADK14861.1 | 300435094 | Clostridium ljungdahli |
| CLJU_c17970 | ADK14860.1 | 300435093 | Clostridium ljungdahli |
| CLJU_c22510 | ADK15311.1 | 300435544 | Clostridium ljungdahli |
| CLJU_c26680 | ADK15726.1 | 300435959 | Clostridium ljungdahli |
| CLJU_c29400 | ADK15988.1 | 300436221 | Clostridium ljungdahli |

Ferredoxin oxidoreductase enzymes transfer electrons from ferredoxins or flavodoxins to NAD(P)H. Two enzymes catalyzing the reversible transfer of electrons from reduced ferredoxins to NAD(P)+ are ferredoxin:NAD+ oxidoreductase (EC 1.18.1.3) and ferredoxin:NADP+ oxidoreductase (FNR, EC 1.18.1.2). Ferredoxin:NADP+ oxidoreductase (FNR, EC 1.18.1.2) has a noncovalently bound FAD cofactor that facilitates the reversible transfer of electrons from NADPH to low-potential acceptors such as ferredoxins or flavodoxins (Blaschkowski et al., Eur. J. Biochem. 123:563-569 (1982); Fujii et al., 1977). The Helicobacter pylori FNR, encoded by HP1164 (fqrB), is coupled to the activity of pyruvate:ferredoxin oxidoreductase (PFOR) resulting in the pyruvate-dependent production of NADPH (St et al. 2007). An analogous enzyme is found in Campylobacter jejuni (St Maurice et al., J. Bacteriol. 189:4764-4773 (2007)). A ferredoxin:NADP+ oxidoreductase enzyme is encoded in the E. coli genome by fpr (Bianchi et al. 1993). Ferredoxin:NAD+ oxidoreductase utilizes reduced ferredoxin to generate NADH from NAD+. In several organisms, including E. coli, this enzyme is a component of multifunctional dioxygenase enzyme complexes. The ferredoxin:NAD+ oxidoreductase of E. coli, encoded by hcaD, is a component of the 3-phenylpropionate dioxygenase system involved in involved in aromatic acid utilization (Diaz et al. 1998). NADH:ferredoxin reductase activity was detected in cell extracts of Hydrogenobacter thermophilus, although a gene with this activity has not yet been indicated (Yoon et al. 2006). Additional ferredoxin:NAD(P)+ oxidoreductases have been annotated in Clostridium carboxydivorans P7. The NADH-dependent reduced ferredoxin: NADP oxidoreductase of C. kluyveri, encoded by nfnAB, catalyzes the concomitant reduction of ferredoxin and NAD+ with two equivalents of NADPH (Wang et al, J Bacteriol 192: 5115-5123 (2010)). Finally, the energy-conserving membrane-associated Rnf-type proteins (Seedorf et al, PNAS 105:2128-2133 (2008); and Herrmann, J. Bacteriol 190:784-791 (2008)) provide a means to generate NADH or NADPH from reduced ferredoxin.

| Protein | GenBank ID | GI Number | Organism |
|---|---|---|---|
| fqrB | NP_207955.1 | 15645778 | Helicobacter pylori |
| fqrB | YP_001482096.1 | 157414840 | Campylobacter jejuni |
| RPA3954 | CAE29395.1 | 39650872 | Rhodopseudomonas palustris |
| Fpr | BAH29712.1 | 225320633 | Hydrogenobacter thermophilus |
| yumC | NP_391091.2 | 255767736 | Bacillus subtilis |
| Fpr | P28861.4 | 399486 | Escherichia coli |
| hcaD | AAC75595.1 | 1788892 | Escherichia coli |
| LOC100282643 | NP_001149023.1 | 226497434 | Zea mays |
| NfnA | YP_001393861.1 | 153953096 | Clostridium kluyveri |
| NfnB | YP_001393862.1 | 153953097 | Clostridium kluyveri |
| CcarbDRAFT_2639 | ZP_05392639.1 | 255525707 | Clostridium carboxidivorans P7 |
| CcarbDRAFT_2638 | ZP_05392638.1 | 255525706 | Clostridium carboxidivorans P7 |
| CcarbDRAFT_2636 | ZP_05392636.1 | 255525704 | Clostridium carboxidivorans P7 |
| CcarbDRAFT_5060 | ZP_05395060.1 | 255528241 | Clostridium carboxidivorans P7 |
| CcarbDRAFT_2450 | ZP_05392450.1 | 255525514 | Clostridium carboxidivorans P7 |
| CcarbDRAFT_1084 | ZP_05391084.1 | 255524124 | Clostridium carboxidivorans P7 |
| RnfC | EDK33306.1 | 146346770 | Clostridium kluyveri |
| RnfD | EDK33307.1 | 146346771 | Clostridium kluyveri |
| RnfG | EDK33308.1 | 146346772 | Clostridium kluyveri |
| RnfE | EDK33309.1 | 146346773 | Clostridium kluyveri |
| RnfA | EDK33310.1 | 146346774 | Clostridium kluyveri |
| RnfB | EDK33311.1 | 146346775 | Clostridium kluyveri |
| CLJU_c11410 (RnfB) | ADK14209.1 | 300434442 | Clostridium ljungdahlii |
| CLJU_c11400 (RnfA) | ADK14208.1 | 300434441 | Clostridium ljungdahlii |
| CLJU_c11390 (RnfE) | ADK14207.1 | 300434440 | Clostridium ljungdahlii |
| CLJU_c11380 (RnfG) | ADK14206.1 | 300434439 | Clostridium ljungdahlii |
| CLJU_c11370 (RnfD) | ADK14205.1 | 300434438 | Clostridium ljungdahlii |
| CLJU_c11360 (RnfC) | ADK14204.1 | 300434437 | Clostridium ljungdahlii |
| MOTH_1518 (NfnA) | YP_430370.1 | 83590361 | Moorella thermoacetica |
| MOTH_1517(NfnB) | YP_430369.1 | 83590360 | Moorella thermoacetica |
| CHY_1992 (NfnA) | YP_360811.1 | 78045020 | Carboxydothermus hydrogenoformans |
| CHY_1993 (NfnB) | YP_360812.1 | 78044266 | Carboxydothermus hydrogenoformans |
| CLJU_c37220 (NfnAB) | YP_003781850.1 | 300856866 | Clostridium ljungdahlii |

### FIG. 3, Step I—Formate Dehydrogenase

Formate dehydrogenase (FDH) catalyzes the reversible transfer of electrons from formate to an acceptor. Enzymes with FDH activity utilize various electron carriers such as, for example, NADH (EC 1.2.1.2), NADPH (EC 1.2.1.43), quinols (EC 1.1.5.6), cytochromes (EC 1.2.2.3) and hydrogenases (EC 1.1.99.33). FDH enzymes have been characterized from Moorella thermoacetica (Andreesen and Ljungdahl, J Bacteriol 116:867-873 (1973); Li et al., J Bacteriol 92:405-412 (1966); Yamamoto et al., J Biol Chem. 258:1826-1832 (1983). The loci, Moth_{—}2312 is responsible for encoding the alpha subunit of formate dehydrogenase while the beta subunit is encoded by Moth_{—}2314 (Pierce et al., Environ Microbiol (2008)). Another set of genes encoding formate dehydrogenase activity with a propensity for CO₂ reduction is encoded by Sfum_{—}2703 through Sfum_{—}2706 in Syntrophobacter fumaroxidans (de Bok et al., Eur J Biochem. 270:2476-2485 (2003)); Reda et al., PNAS 105:10654-10658 (2008)). A similar set of genes presumed to carry out the same function are encoded by CHY_{—}0731, CHY_{—}0732, and CHY_{—}0733 in C. hydrogenoformans (Wu et al., PLoS Genet 1:e65 (2005)). Formate dehydrogenases are also found many additional organisms including C. carboxidivorans P7, Bacillus methanolicus, Burkholderia stabilis, Moorella thermoacetica ATCC 39073, Candida boidinii, Candida methylica, and Saccharomyces cerevisiae S288c. The soluble formate dehydrogenase from Ralstonia eutropha reduces NAD⁺ (fdsG, -B, -A, -C, -D) (Oh and Bowien, 1998)

| Protein | GenBank ID | GI Number | Organism |
|---|---|---|---|
| Moth_2312 | YP_431142 | 148283121 | Moorella thermoacetica |
| Moth_2314 | YP_431144 | 83591135 | Moorella thermoacetica |
| Sfum_2703 | YP_846816.1 | 116750129 | Syntrophobacter fumaroxidans |
| Sfum_2704 | YP_846817.1 | 116750130 | Syntrophobacter fumaroxidans |
| Sfum_2705 | YP_846818.1 | 116750131 | Syntrophobacter fumaroxidans |
| Sfum_2706 | YP_846819.1 | 116750132 | Syntrophobacter fumaroxidans |
| CHY_0731 | YP_359585.1 | 78044572 | Carboxydothermus hydrogenoformans |
| CHY_0732 | YP_359586.1 | 78044500 | Carboxydothermus hydrogenoformans |
| CHY_0733 | YP_359587.1 | 78044647 | Carboxydothermus hydrogenoformans |
| CcarbDRAFT_0901 | ZP_05390901.1 | 255523938 | Clostridium carboxidivorans P7 |
| CcarbDRAFT_4380 | ZP_05394380.1 | 255527512 | Clostridium carboxidivorans P7 |
| fdhA, MGA3_06625 | EIJ82879.1 | 387590560 | Bacillus methanolicus MGA3 |
| fdhA, PB1_11719 | ZP_10131761.1 | 387929084 | Bacillus methanolicus PB1 |
| fdhD, MGA3_06630 | EIJ82880.1 | 387590561 | Bacillus methanolicus MGA3 |
| fdhD, PB1_11724 | ZP_10131762.1 | 387929085 | Bacillus methanolicus PB1 |
| fdh | ACF35003. | 194220249 | Burkholderia stabilis |
| FDH1 | AAC49766.1 | 2276465 | Candida boidinii |
| fdh | CAA57036.1 | 1181204 | Candida methylica |
| FDH2 | P0CF35.1 | 294956522 | Saccharomyces cerevisiae S288c |
| FDH1 | NP_015033.1 | 6324964 | Saccharomyces cerevisiae S288c |
| fdsG | YP_725156.1 | 113866667 | Ralstonia eutropha |
| fdsB | YP_725157.1 | 113866668 | Ralstonia eutropha |
| fdsA | YP_725158.1 | 113866669 | Ralstonia eutropha |
| fdsC | YP_725159.1 | 113866670 | Ralstonia eutropha |
| fdsD | YP_725160.1 | 113866671 | Ralstonia eutropha |

### FIG. 3, Step J—Methanol Dehydrogenase

NAD+ dependent methanol dehydrogenase enzymes (EC 1.1.1.244) catalyze the conversion of methanol and NAD+ to formaldehyde and NADH. An enzyme with this activity was first characterized in Bacillus methanolicus (Heggeset, et al., Applied and Environmental Microbiology, 78(15):5170-5181 (2012)). This enzyme is zinc and magnesium dependent, and activity of the enzyme is enhanced by the activating enzyme encoded by act (Kloosterman et al, J Biol Chem 277:34785-92 (2002)). Additional NAD(P)+ dependent enzymes can be identified by sequence homology. Methanol dehydrogenase enzymes utilizing different electron acceptors are also known in the art. Examples include cytochrome dependent enzymes such as mxaIF of the methylotroph Methylobacterium extorquens (Nunn et al, Nucl Acid Res 16:7722 (1988)). Methanol dehydrogenase enzymes of methanotrophs such as Methylococcus capsulatis function in a complex with methane monooxygenase (MMO) (Myronova et al, Biochem 45:11905-14 (2006)). Methanol can also be oxidized to formaldehyde by alcohol oxidase enzymes such as methanol oxidase (EC 1.1.3.13) of Candida boidinii (Sakai et al, Gene 114: 67-73 (1992)).

| Protein | GenBank ID | GI Number | Organism |
|---|---|---|---|
| mdh, | EIJ77596.1 | 387585261 | Bacillus methanolicus |
| MGA3_17392 | | | MGA3 |
| mdh2, | EIJ83020.1 | 387590701 | Bacillus methanolicus |
| MGA3_07340 | | | MGA3 |
| mdh3, | EIJ80770.1 | 387588449 | Bacillus methanolicus |
| MGA3_10725 | | | MGA3 |
| act, | EIJ83380.1 | 387591061 | Bacillus methanolicus |
| MGA3_09170 | | | MGA3 |
| mdh, | ZP_10132907.1 | 387930234 | Bacillus methanolicus |
| PB1_17533 | | | PB1 |
| mdh1, | ZP_10132325.1 | 387929648 | Bacillus methanolicus |
| PB1_14569 | | | PB1 |
| mdh2, | ZP_10131932.1 | 387929255 | Bacillus methanolicus |
| PB1_12584 | | | PB1 |
| act, | ZP_10132290.1 | 387929613 | Bacillus methanolicus |
| PB1_14394 | | | PB1 |
| BFZC1_05383 | ZP_07048751.1 | 299535429 | Lysinibacillus fusiformis |
| BFZC1_20163 | ZP_07051637.1 | 299538354 | Lysinibacillus fusiformis |
| Bsph_4187 | YP_001699778.1 | 169829620 | Lysinibacillus sphaericus |
| Bsph_1706 | YP_001697432.1 | 169827274 | Lysinibacillus sphaericus |
| MCA0299 | YP_112833.1 | 53802410 | Methylococcus capsulatis |
| MCA0782 | YP_113284.1 | 53804880 | Methylococcus capsulatis |
| mxaI | YP_002965443.1 | 240140963 | Methylobacterium |
| | | | extorquens |
| mxaF | YP_002965446.1 | 240140966 | Methylobacterium |
| | | | extorquens |
| AOD1 | AAA34321.1 | 170820 | Candida boidinii |

### FIG. 3, Step K—Spontaneous or Formaldehyde Activating Enzyme

The conversion of formaldehyde and THF to methylenetetrahydrofolate can occur spontaneously. It is also possible that the rate of this reaction can be enhanced by a formaldehyde activating enzyme. A formaldehyde activating enzyme (Fae) has been identified in Methylobacterium extorquens AM1 which catalyzes the condensation of formaldehyde and tetrahydromethanopterin to methylene tetrahydromethanopterin (Vorholt, et al., J. Bacteriol., 182(23), 6645-6650 (2000)). It is possible that a similar enzyme exists or can be engineered to catalyze the condensation of formaldehyde and tetrahydrofolate to methylenetetrahydrofolate. Homologs exist in several organisms including Xanthobacter autotrophicus Py2 and Hyphomicrobium denitrificans ATCC 51888.

| Protein | GenBank ID | GI Number | Organism |
|---|---|---|---|
| MexAM1_META1p1766 | Q9FA38.3 | 17366061 | Methylobacterium extorquens AM1 |
| Xaut_0032 | YP_001414948.1 | 154243990 | Xanthobacter autotrophicus Py2 |
| Hden_1474 | YP_003755607.1 | 300022996 | Hyphomicrobium denitrificans |
| | | | ATCC 51888 |

### FIG. 3, Step L—Formaldehyde Dehydrogenase

Oxidation of formaldehyde to formate is catalyzed by formaldehyde dehydrogenase. An NAD+ dependent formaldehyde dehydrogenase enzyme is encoded by fdhA of Pseudomonas putida (Ito et al, J Bacteriol 176: 2483-2491 (1994)). Additional formaldehyde dehydrogenase enzymes include the NAD+ and glutathione independent formaldehyde dehydrogenase from Hyphomicrobium zavarzinii (Jerome et al, Appl Microbiol Biotechnol 77:779-88 (2007)), the glutathione dependent formaldehyde dehydrogenase of Pichia pastoris (Sunga et al, Gene 330:39-47 (2004)) and the NAD(P)+ dependent formaldehyde dehydrogenase of Methylobacter marinus (Speer et al, FEMS Microbiol Lett, 121(3):349-55 (1994)).

| Protein | GenBank ID | GI Number | Organism |
|---|---|---|---|
| fdhA | P46154.3 | 1169603 | Pseudomonas putida |
| faoA | CAC85637.1 | 19912992 | Hyphomicrobium zavarzinii |
| Fld1 | CCA39112.1 | 328352714 | Pichia pastoris |
| fdh | P47734.2 | 221222447 | Methylobacter marinus |

In addition to the formaldehyde dehydrogenase enzymes listed above, alternate enzymes and pathways for converting formaldehyde to formate are known in the art. For example, many organisms employ glutathione-dependent formaldehyde oxidation pathways, in which formaldehyde is converted to formate in three steps via the intermediates S-hydroxymethylglutathione and S-formylglutathione (Vorholt et al, J Bacteriol 182:6645-50 (2000)). The enzymes of this pathway are S-(hydroxymethyl)glutathione synthase (EC 4.4.1.22), glutathione-dependent formaldehyde dehydrogenase (EC 1.1.1.284) and S-formylglutathione hydrolase (EC 3.1.2.12).

### FIG. 3, Step M—Spontaneous or S-(Hydroxymethyl)Glutathione Synthase

While conversion of formaldehyde to S-hydroxymethylglutathione can occur spontaneously in the presence of glutathione, it has been shown by Goenrich et al (Goenrich, et al., J Biol Chem 277(5); 3069-72 (2002)) that an enzyme from Paracoccus denitrificans can accelerate this spontaneous condensation reaction. The enzyme catalyzing the conversion of formaldehyde and glutathione was purified and named glutathione-dependent formaldehyde-activating enzyme (Gfa). The gene encoding it, which was named gfa, is located directly upstream of the gene for glutathione-dependent formaldehyde dehydrogenase, which catalyzes the subsequent oxidation of S-hydroxymethylglutathione. Putative proteins with sequence identity to Gfa from P. denitrificans are present also in Rhodobacter sphaeroides, Sinorhizobium meliloti, and Mesorhizobium loti.

| Protein | GenBank ID | GI Number | Organism |
|---|---|---|---|
| Gfa | Q51669.3 | 38257308 | Paracoccus denitrificans |
| Gfa | ABP71667.1 | 145557054 | Rhodobacter sphaeroides ATCC 17025 |
| Gfa | Q92WX6.1 | 38257348 | Sinorhizobium meliloti 1021 |
| Gfa | Q98LU4.2 | 38257349 | Mesorhizobium loti MAFF303099 |

### FIG. 3, Step N—Glutathione-Dependent Formaldehyde Dehydrogenase

Glutathione-dependent formaldehyde dehydrogenase (GS-FDH) belongs to the family of class III alcohol dehydrogenases. Glutathione and formaldehyde combine non-enzymatically to form hydroxymethylglutathione, the true substrate of the GS-FDH catalyzed reaction. The product, S-formylglutathione, is further metabolized to formic acid.

| Protein | GenBank ID | GI Number | Organism |
|---|---|---|---|
| frmA | YP_488650.1 | 388476464 | Escherichia coli K-12 MG1655 |
| SFA1 | NP_010113.1 | 6320033 | Saccharomyces cerevisiae S288c |
| flhA | AAC44551.1 | 1002865 | Paracoccus denitrificans |
| adhI | AAB09774.1 | 986949 | Rhodobacter sphaeroides |

### FIG. 3, Step O—S-Formylglutathione Hydrolase

S-formylglutathione hydrolase is a glutathione thiol esterase found in bacteria, plants and animals. It catalyzes conversion of S-formylglutathione to formate and glutathione. The fghA gene of P. denitrificans is located in the same operon with gfa and flhA, two genes involved in the oxidation of formaldehyde to formate in this organism. In E. coli, FrmB is encoded in an operon with FrmR and FrmA, which are proteins involved in the oxidation of formaldehyde. YeiG of E. coli is a promiscuous serine hydrolase; its highest specific activity is with the substrate S-formylglutathione.

| Protein | GenBank ID | GI Number | Organism |
|---|---|---|---|
| frmB | NP_414889.1 | 16128340 | Escherichia coli K-12 MG1655 |
| yeiG | AAC75215.1 | 1788477 | Escherichia coli K-12 MG1655 |
| fghA | AAC44554.1 | 1002868 | Paracoccus denitrificans |

### FIG. 3, Step P—Carbon Monoxide Dehydrogenase (CODH)

CODH is a reversible enzyme that interconverts CO and CO₂ at the expense or gain of electrons. The natural physiological role of the CODH in ACS/CODH complexes is to convert CO₂ to CO for incorporation into acetyl-CoA by acetyl-CoA synthase. Nevertheless, such CODH enzymes are suitable for the extraction of reducing equivalents from CO due to the reversible nature of such enzymes. Expressing such CODH enzymes in the absence of ACS allows them to operate in the direction opposite to their natural physiological role (i.e., CO oxidation).

In M. thermoacetica, C. hydrogenoformans, C. carboxidivorans P7, and several other organisms, additional CODH encoding genes are located outside of the ACS/CODH operons. These enzymes provide a means for extracting electrons (or reducing equivalents) from the conversion of carbon monoxide to carbon dioxide. The M. thermoacetica gene (Genbank Accession Number: YP_{—}430813) is expressed by itself in an operon and is believed to transfer electrons from CO to an external mediator like ferredoxin in a “Ping-pong” reaction. The reduced mediator then couples to other reduced nicolinamide adenine dinucleotide phosphate (NAD(P)H) carriers or ferredoxin-dependent cellular processes (Ragsdale, Annals of the New York Academy of Sciences 1125: 129-136 (2008)). The genes encoding the C. hydrogenoformans CODH-II and CooF, a neighboring protein, were cloned and sequenced (Gonzalez and Robb, FEMS Microbiol Lett. 191:243-247 (2000)). The resulting complex was membrane-bound, although cytoplasmic fractions of CODH-II were shown to catalyze the formation of NADPH suggesting an anabolic role (Svetlitchnyi et al., J Bacteriol. 183:5134-5144 (2001)). The crystal structure of the CODH-II is also available (Dobbek et al., Science 293:1281-1285 (2001)). Similar ACS-free CODH enzymes can be found in a diverse array of organisms including Geobacter metallireducens GS-15, Chlorobium phaeobacteroides DSM 266, Clostridium cellulolyticum H10, Desulfovibrio desulfuricans subsp. desulfuricans str. ATCC 27774, Pelobacter carbinolicus DSM 2380, C. ljungdahli and Campylobacter curvus 525.92.

| Protein | GenBank ID | GI Number | Organism |
|---|---|---|---|
| CODH (putative) | YP_430813 | 83590804 | Moorella thermoacetica |
| CODH-II (CooS-II) | YP_358957 | 78044574 | Carboxydothermus hydrogenoformans |
| CooF | YP_358958 | 78045112 | Carboxydothermus hydrogenoformans |
| CODH (putative) | ZP_05390164.1 | 255523193 | Clostridium carboxidivorans P7 |
| CcarbDRAFT_0341 | ZP_05390341.1 | 255523371 | Clostridium carboxidivorans P7 |
| CcarbDRAFT_1756 | ZP_05391756.1 | 255524806 | Clostridium carboxidivorans P7 |
| CcarbDRAFT_2944 | ZP_05392944.1 | 255526020 | Clostridium carboxidivorans P7 |
| CODH | YP_384856.1 | 78223109 | Geobacter metallireducens GS-15 |
| Cpha266_0148 | YP_910642.1 | 119355998 | Chlorobium phaeobacteroides DSM 266 |
| (cytochrome c) | | | |
| Cpha266_0149 (CODH) | YP_910643.1 | 119355999 | Chlorobium phaeobacteroides DSM 266 |
| Ccel_0438 | YP_002504800.1 | 220927891 | Clostridium cellulolyticum H10 |
| Ddes_0382 (CODH) | YP_002478973.1 | 220903661 | Desulfovibrio desulfuricans subsp. |
| | | | desulfuricans str. ATCC 27774 |
| Ddes_0381 (CooC) | YP_002478972.1 | 220903660 | Desulfovibrio desulfuricans subsp. |
| | | | desulfuricans str. ATCC 27774 |
| Pcar_0057 (CODH) | YP_355490.1 | 7791767 | Pelobacter carbinolicus DSM 2380 |
| Pcar_0058 (CooC) | YP_355491.1 | 7791766 | Pelobacter carbinolicus DSM 2380 |
| Pcar_0058 (HypA) | YP_355492.1 | 7791765 | Pelobacter carbinolicus DSM 2380 |
| CooS (CODH) | YP_001407343.1 | 154175407 | Campylobacter curvus 525.92 |
| CLJU_c09110 | ADK13979.1 | 300434212 | Clostridium ljungdahli |
| CLJU_c09100 | ADK13978.1 | 300434211 | Clostridium ljungdahli |
| CLJU_c09090 | ADK13977.1 | 300434210 | Clostridium ljungdahli |

### Example III

### Methods for Formaldehyde Fixation

Provided herein are exemplary pathways, which utilize formaldehyde produced from the oxidation of methanol (see, e.g., FIG. 1, step A, or FIG. 3, step J) or from formate assimilation pathways described in Example I (see, e.g., FIG. 1) in the formation of intermediates of certain central metabolic pathways that can be used for the production of compounds disclosed herein.

One exemplary pathway that can utilize formaldehyde produced from the oxidation of methanol is shown in FIG. 1, which involves condensation of formaldehyde and D-ribulose-5-phosphate to form hexulose-6-phosphate (h6p) by hexulose-6-phosphate synthase (FIG. 1, step B). The enzyme can use Mg²⁺ or Mn²⁺ for maximal activity, although other metal ions are useful, and even non-metal-ion-dependent mechanisms are contemplated. H6p is converted into fructose-6-phosphate by 6-phospho-3-hexuloisomerase (FIG. 1, step C).

Another exemplary pathway that involves the detoxification and assimilation of formaldehyde produced from the oxidation of methanol is shown in FIG. 1 and proceeds through dihydroxyacetone. Dihydroxyacetone synthase is a special transketolase that first transfers a glycoaldehyde group from xylulose-5-phosphate to formaldehyde, resulting in the formation of dihydroxyacetone (DHA) and glyceraldehyde-3-phosphate (G3P), which is an intermediate in glycolysis (FIG. 1). The DHA obtained from DHA synthase can be further phosphorylated to form DHA phosphate and assimilated into glycolysis and several other pathways (FIG. 1).

### FIG. 1, Steps B and C—Hexulose-6-Phosphate Synthase (Step B) and 6-Phospho-3-Hexuloisomerase (Step C)

Both of the hexulose-6-phosphate synthase and 6-phospho-3-hexuloisomerase enzymes are found in several organisms, including methanotrops and methylotrophs where they have been purified (Kato et al., 2006, BioSci Biotechnol Biochem. 70(1):10-21. In addition, these enzymes have been reported in heterotrophs such as Bacillus subtilis also where they are reported to be involved in formaldehyde detoxification (Mitsui et al., 2003, AEM 69(10):6128-32, Yasueda et al., 1999. J Bac 181(23):7154-60. Genes for these two enzymes from the methylotrophic bacterium Mycobacterium gastri MB19 have been fused and E. coli strains harboring the hps-phi construct showed more efficient utilization of formaldehyde (Orita et al., 2007, Appl Microbiol Biotechnol. 76:439-445). In some organisms, these two enzymes naturally exist as a fused version that is bifunctional.

Exemplary candidate genes for hexulose-6-phopshate synthase are:

| Protein | GenBank ID | GI number | Organism |
|---|---|---|---|
| Hps | AAR39392.1 | 40074227 | Bacillus methanolicus MGA3 |
| Hps | EIJ81375.1 | 387589055 | Bacillus methanolicus PB1 |
| RmpA | BAA83096.1 | 5706381 | Methylomonas aminofaciens |
| RmpA | BAA90546.1 | 6899861 | Mycobacterium gastri |
| YckG | BAA08980.1 | 1805418 | Bacillus subtilis |

Exemplary gene candidates for 6-phospho-3-hexuloisomerase are:

| Protein | GenBank ID | GI number | Organism |
|---|---|---|---|
| Phi | AAR39393.1 | 40074228 | Bacillus methanolicus MGA3 |
| Phi | EIJ81376.1 | 387589056 | Bacillus methanolicus PB1 |
| Phi | BAA83098.1 | 5706383 | Methylomonas aminofaciens |
| RmpB | BAA90545.1 | 6899860 | Mycobacterium gastri |

Candidates for enzymes where both of these functions have been fused into a single open reading frame include the following.

| Protein | GenBank ID | GI number | Organism |
|---|---|---|---|
| PH1938 | NP_143767.1 | 14591680 | Pyrococcus horikoshii OT3 |
| PF0220 | NP_577949.1 | 18976592 | Pyrococcus furiosus |
| TK0475 | YP_182888.1 | 57640410 | Thermococcus kodakaraensis |
| | NP_127388.1 | 14521911 | Pyrococcus abyssi |
| MCA2738 | YP_115138.1 | 53803128 | Methylococcus capsulatas |

### FIG. 1, Step D—Dihydroxyacetone Synthase

The dihydroxyacetone synthase enzyme in Candida boidinii uses thiamine pyrophosphate and Mg²⁺ as cofactors and is localized in the peroxisome. The enzyme from the methanol-growing carboxydobacterium, Mycobacter sp. strain JC1 DSM 3803, was also found to have DHA synthase and kinase activities (Ro et al., 1997, JBac 179(19):6041-7). DHA synthase from this organism also has similar cofactor requirements as the enzyme from C. boidinii. The Kₘs for formaldehyde and xylulose 5-phosphate were reported to be 1.86 mM and 33.3 microM, respectively. Several other mycobacteria, excluding only Mycobacterium tuberculosis, can use methanol as the sole source of carbon and energy and are reported to use dihydroxyacetone synthase (Part et al., 2003, JBac 185(1):142-7.

| Protein | GenBank ID | GI number | Organism |
|---|---|---|---|
| DAS1 | AAC83349.1 | 3978466 | Candida boidinii |
| HPODL_2613 | EFW95760.1 | 320581540 | Ogataea parapolymorpha DL-1 |
| | | | (Hansenula polymorpha DL-1) |
| | AAG12171.2 | 18497328 | Mycobacter sp. strain JC1 |
| | | | DSM 3803 |

### Example IV

### Pathways to 1,3-Butanediol and Crotyl Alcohol

Pathways to product 1,3-butanediol and crotyl alcohol that utilize the acetyl-CoA produced by the formate assimilation and formaldehyde fixation pathways described herein are shown in FIG. 10. These pathways can begin with the initiation of fatty acid biosynthesis, in which malonyl-ACP is condensed with acetyl-CoA or acetyl-ACP to form acetoacetyl-ACP (step A). The second step involves reduction of acetoacetyl-ACP to 3-hydroxybutyryl-ACP. Following dehydration to crotonyl-ACP and another reduction, butyryl-ACP is formed. The chain elongation typically continues with further addition of malonyl-ACP until a long-chain acyl chain is formed, which is then hydrolyzed by a thioesterase into a free C16 fatty acid. Bacterial fatty acid synthesis systems (FAS II) utilize discreet proteins for each step, whereas fungal and mammalian fatty acid synthesis systems (FAS I) utilize complex multifunctional proteins. The pathways utilize one or more enzymes of fatty acid biosynthesis to produce the C3 and C4 products 1,3-butanediol and crotyl alcohol.

Several pathways are shown in FIG. 10 for converting acetoacetyl-ACP to 1,3-butanediol. In some pathways, acetoacetyl-ACP is first converted to acetoacetyl-CoA (step D). Alternatively, acetoacetyl-CoA can also be synthesized from acetyl-CoA and malonyl-CoA by acetoacetyl-CoA synthase (EC 2.3.1.194). Additionally, acetyl-CoA can be convert to malonyl-CoA using an acetyl-CoA carboxylase (step T of FIG. 1). Acetoacetyl-CoA can then be hydrolyzed to acetoacetate by a CoA transferase, hydrolase or synthetase (step E of FIG. 10). Acetoacetate is then reduced to 3-oxobutyraldehyde by a carboxylic acid reductase (step F of FIG. 10). Alternately, acetoacetyl-CoA is converted directly to 3-oxobutyraldehyde by a CoA-dependent aldehyde dehydrogenase (step I of FIG. 10). In yet another embodiment, acetoacetyl-ACP is converted directly to 3-oxobutyraldehyde by an acyl-ACP reductase (step J of FIG. 10). 3-Oxobutyraldehyde is further reduced to 1,3-butanediol via a 4-hydroxy-2-butanone or 3-hydroxybutyraldehyde intermediate (steps G and S, or steps R and AA of FIG. 10). Another option is the direct conversion of acetoacetyl-CoA to 4-hydroxy-2-butanone by a bifunctional enzyme with aldehyde dehydrogenase/alcohol dehydrogenase activity (step K of FIG. 10). Pathways to 1,3-butanediol can also proceed through a 3-hydroxybutyryl-CoA intermediate. This intermediate is formed by the reduction of acetoacetyl-CoA (step P of FIG. 10) or the transacylation of 3-hydroxybutyryl-ACP (step X of FIG. 10). 3-Hydroxybutyryl-CoA is further converted to 3-hydroxybutyrate (step Y of FIG. 10), 3-hydroxybutyraldehyde (step N of FIG. 10) or 1,3-butanediol (step O of FIG. 10). Alternately, the 3-hydroxybutyrate intermediate is formed from acetoacetate (step Q of FIG. 10) or via hydrolysis of 3-hydroxybutyryl-ACP (step L of FIG. 10). The 3-hydroxybutyraldehyde intermediate is also the product of 3-hydroxybutyryl-ACP reductase (step M of FIG. 10).

FIG. 10 also shows pathways from malonyl-ACP to crotyl alcohol. In one embodiment, fatty acid initiation and extension enzymes produce the crotonyl-ACP intermediate (steps A, B, C). Crotonyl-ACP is then transacylated, hydrolyzed or reduced to crotonyl-CoA, crotonate or crotonaldehyde, respectively (steps AE, T, U). Crotonyl-CoA and crotonate are interconverted by a CoA hydrolase, transferase or synthetase (step AF). Crotonate is reduced to crotonaldehyde by a carboxylic acid reductase (step AG). In the final step of all pathways, crotonaldehyde is reduced to crotyl alcohol by an aldehyde reductase in step AH. Numerous alternate pathways enumerated in the table below are also encompassed in the invention. Crotonyl-CoA can be reduced to crotonaldehyde or crotyl alcohol (steps V, W). Alternately, the 3-hydroxybutyryl intermediates of the previously described 1,3-butanediol pathways can also be converted to crotyl alcohol precursors. For example, dehydration of 3-hydroxybutyryl-CoA, 3-hydroxybutyrate or 3-hydroxybutyraldehyde yields crotonyl-CoA, crotonate or crotonaldehyde, respectively (step AB, AC, AD).

FIG. 10 still further shows pathways for production of 1,3-butadiol and crotyl alcohol which can include the conversion of two acetyl-CoA molecules to acetoacetyl-CoA by an acetyl-CoA:acetyl-CoA acyltransferase. FIG. 10 still further shows pathways that include the conversion of 4-hydroxybutyryl-CoA to crotonyl-CoA by a 4-hydroxybutyryl-CoA dehydratase.

Several of the enzyme activities required for the reactions shown in FIG. 10 are listed in the table below.

| Label | Function | Step |
|---|---|---|
| 1.1.1.a | Oxidoreductase (oxo to alcohol) | 10B, 10G, 10P, 10Q, 10R, 10S, 10AA, 10AH |
| 1.1.1.c | Oxidoreductase (acyl-CoA to alcohol) | 10K, 10O, 10W |
| 1.2.1.b | Oxidoreductase (acyl-CoA to aldehyde) | 10I, 10N, 10V |
| 1.2.1.e | Oxidoreductase (acid to aldehyde) | 10F, 10Z, 10AG |
| 1.2.1.f | Oxidoreductase (acyl-ACP to aldehyde) | 10J, 10M, 10U |
| 2.3.1.e | Acyl-ACP C-acyltransferase (decarboxylating) | 10A |
| 2.3.1.f | CoA-ACP acyltransferase | 10D, 10X, 10AE, |
| 2.3.1.g | Fatty-acid synthase | 10A, 10B, 10C, |
| 2.8.3.a | CoA transferase | 10E, 10Y, 10AF |
| 3.1.2.a | CoA hydrolase | 10E, 10Y, 10AF |
| 3.1.2.b | Acyl-ACP thioesterase | 10H, 10L, 10T, |
| 4.2.1.a | Hydro-lyase | 10C, 10AB, 10AC, 10AD |
| 6.2.1.a | CoA synthetase | 10E, 10Y, 10AF |

### 1.1.1.a Oxidoreductase (Oxo to Alcohol)

Several reactions shown in FIG. 10 are catalyzed by alcohol dehydrogenase enzymes. These reactions include Steps B, G, P, Q, R, S, AA and AH. Exemplary alcohol dehydrogenase enzymes are described in further detail below.

The reduction of glutarate semialdehyde to 5-hydroxyvalerate by glutarate semialdehyde reductase entails reduction of an aldehyde to its corresponding alcohol. Enzymes with glutarate semialdehyde reductase activity include the ATEG_{—}00539 gene product of Aspergillus terreus and 4-hydroxybutyrate dehydrogenase of Arabidopsis thaliana, encoded by 4hbd (WO 2010/068953A2). The A. thaliana enzyme was cloned and characterized in yeast (Breitkreuz et al., J. Biol. Chem. 278:41552-41556 (2003)).

| PROTEIN | GENBANK ID | GI NUMBER | ORGANISM |
|---|---|---|---|
| ATEG_00539 | XP_001210625.1 | 115491995 | Aspergillus terreus |
| | | | NIH2624 |
| 4hbd | AAK94781.1 | 15375068 | Arabidopsis thaliana |

Additional genes encoding enzymes that catalyze the reduction of an aldehyde to alcohol (i.e., alcohol dehydrogenase or equivalently aldehyde reductase) include alrA encoding a medium-chain alcohol dehydrogenase for C2-C14 (Tani et al., Appl. Environ. Microbiol. 66:5231-5235 (2000)), yqhD and fucO from E. coli (Sulzenbacher et al., 342:489-502 (2004)), and bdh I and bdh II from C. acetobutylicum which converts butyraldehyde into butanol (Walter et al., 174:7149-7158 (1992)). YqhD catalyzes the reduction of a wide range of aldehydes using NADPH as the cofactor, with a preference for chain lengths longer than C(3) (Sulzenbacher et al., 342:489-502 (2004); Perez et al., J Biol. Chem. 283:7346-7353 (2008)). The adhA gene product from Zymomonas mobilisE has been demonstrated to have activity on a number of aldehydes including formaldehyde, acetaldehyde, propionaldehyde, butyraldehyde, and acrolein (Kinoshita et al., Appl Microbiol Biotechnol 22:249-254 (1985)). Additional aldehyde reductase candidates are encoded by bdh in C. saccharoperbutylacetonicum and Cbei_{—}1722, Cbei_{—}2181 and Cbei_{—}2421 in C. Beijerinckii. Additional aldehyde reductase gene candidates in Saccharomyces cerevisiae include the aldehyde reductases GRE3, ALD2-6 and HFD1, glyoxylate reductases GOR1 and YPL113C and glycerol dehydrogenase GCY1 (WO 2011/022651A1; Atsumi et al., Nature 451:86-89 (2008)). The enzyme candidates described previously for catalyzing the reduction of methylglyoxal to acetol or lactaldehyde are also suitable lactaldehyde reductase enzyme candidates.

| Protein | GENBANK ID | GI NUMBER | ORGANISM |
|---|---|---|---|
| alrA | BAB12273.1 | 9967138 | Acinetobacter sp. strain M-1 |
| ADH2 | NP_014032.1 | 6323961 | Saccharomyces cerevisiae |
| yqhD | NP_417484.1 | 16130909 | Escherichia coli |
| fucO | NP_417279.1 | 16130706 | Escherichia coli |
| bdh I | NP_349892.1 | 15896543 | Clostridium acetobutylicum |
| bdh II | NP_349891.1 | 15896542 | Clostridium acetobutylicum |
| adhA | YP_162971.1 | 56552132 | Zymomonas mobilis |
| bdh | BAF45463.1 | 124221917 | Clostridium |
| | | | saccharoperbutylacetonicum |
| Cbei _1722 | YP_001308850 | 150016596 | Clostridium beijerinckii |
| Cbei_2181 | YP_001309304 | 150017050 | Clostridium beijerinckii |
| Cbei_2421 | YP_001309535 | 150017281 | Clostridium beijerinckii |
| GRE3 | P38715.1 | 731691 | Saccharomyces cerevisiae |
| ALD2 | CAA89806.1 | 825575 | Saccharomyces cerevisiae |
| ALD3 | NP_013892.1 | 6323821 | Saccharomyces cerevisiae |
| ALD4 | NP_015019.1 | 6324950 | Saccharomyces cerevisiae |
| ALD5 | NP_010996.2 | 330443526 | Saccharomyces cerevisiae |
| ALD6 | ABX39192.1 | 160415767 | Saccharomyces cerevisiae |
| HFD1 | Q04458.1 | 2494079 | Saccharomyces cerevisiae |
| GOR1 | NP_014125.1 | 6324055 | Saccharomyces cerevisiae |
| YPL113C | AAB68248.1 | 1163100 | Saccharomyces cerevisiae |
| GCY1 | CAA99318.1 | 1420317 | Saccharomyces cerevisiae |

Enzymes exhibiting 4-hydroxybutyrate dehydrogenase activity (EC 1.1.1.61) also fall into this category. Such enzymes have been characterized in Ralstonia eutropha (Bravo et al., J Forens Sci, 49:379-387 (2004)) and Clostridium kluyveri (Wolff et al., Protein Expr. Purif 6:206-212 (1995)). Yet another gene is the alcohol dehydrogenase adhI from Geobacillus therinoglucosidasius (Jeon et al., J Biotechnol 135:127-133 (2008)).

| PROTEIN | GENBANK ID | GI NUMBER | ORGANISM |
|---|---|---|---|
| 4hbd | YP_726053.1 | 113867564 | Ralstonia eutropha H16 |
| 4hbd | L21902.1 | 146348486 | Clostridium kluyveri DSM 555 |
| adhI | AAR91477.1 | 40795502 | Geobacillus |
| | | | therinoglucosidasius |

Another exemplary aldehyde reductase is methylmalonate semialdehyde reductase, also known as 3-hydroxyisobutyrate dehydrogenase (EC 1.1.1.31). This enzyme participates in valine, leucine and isoleucine degradation and has been identified in bacteria, eukaryotes, and mammals. The enzyme encoded by P84067 from Thermus thermophilus HB8 has been structurally characterized (Lokanath et al., J Mol Biol, 352:905-17 (2005)). The reversibility of the human 3-hydroxyisobutyrate dehydrogenase was demonstrated using isotopically-labeled substrate (Manning et al., Biochem J, 231:481-4 (1985)). Additional genes encoding this enzyme include 3hidh in Homo sapiens (Hawes et al., Methods Enzymol, 324:218-228 (2000)) and Oryctolagus cuniculus (Hawes et al., supra; Chowdhury et al., Biosci. Biotechnol Biochem. 60:2043-2047 (1996)), minsB in Pseudomonas aeruginosa and Pseudomonas putida, and dhat in Pseudomonas putida (Aberhart et al., J Chem. Soc. [Perkin 1] 6:1404-1406 (1979); Chowdhury et al., Biosci. Biotechnol Biochem. 60:2043-2047 (1996); Chowdhury et al., Biosci. Biotechnol Biochem. 67:438-441 (2003)). Several 3-hydroxyisobutyrate dehydrogenase enzymes have been characterized in the reductive direction, including mmsB from Pseudomonas aeruginosa (Gokarn et al., U.S. Pat. No. 739,676, (2008)) and minsB from Pseudomonas putida.

| PROTEIN | GENBANK ID | GI NUMBER | ORGANISM |
|---|---|---|---|
| P84067 | P84067 | 75345323 | Thermus thermophilus |
| 3hidh | P31937.2 | 12643395 | Homo sapiens |
| 3hidh | P32185.1 | 416872 | Oryctolagus cuniculus |
| nunsB | NP_746775.1 | 26991350 | Pseudomonas putida |
| nunsB | P28811.1 | 127211 | Pseudomonas aeruginosa |
| dhat | Q59477.1 | 2842618 | Pseudomonas putida |

There exist several exemplary alcohol dehydrogenases that convert a ketone to a hydroxyl functional group. Two such enzymes from E. coli are encoded by malate dehydrogenase (mdh) and lactate dehydrogenase (ldhA). In addition, lactate dehydrogenase from Ralstonia eutropha has been shown to demonstrate high activities on 2-ketoacids of various chain lengths including lactate, 2-oxobutyrate, 2-oxopentanoate and 2-oxoglutarate (Steinbuchel et al., Eur. J. Biochem. 130:329-334 (1983)). Conversion of alpha-ketoadipate into alpha-hydroxyadipate can be catalyzed by 2-ketoadipate reductase, an enzyme reported to be found in rat and in human placenta (Suda et al., Arch. Biochem. Biophys. 176:610-620 (1976); Suda et al., Biochem. Biophys. Res. Commun. 77:586-591 (1977)). An additional oxidoreductase is the mitochondrial 3-hydroxybutyrate dehydrogenase (bdh) from the human heart which has been cloned and characterized (Marks et al., J. Biol. Chem. 267:15459-15463 (1992)). Alcohol dehydrogenase enzymes of C. beijerinckii (Ismaiel et al., J. Bacteriol. 175:5097-5105 (1993)) and T. brockii (Lamed et al., Biochem. J. 195:183-190 (1981); Peretz et al., Biochemistry. 28:6549-6555 (1989)) convert acetone to isopropanol. Methyl ethyl ketone reductase catalyzes the reduction of MEK to 2-butanol. Exemplary MEK reductase enzymes can be found in Rhodococcus ruber (Kosjek et al., Biotechnol Bioeng. 86:55-62 (2004)) and Pyrococcus furiosus (van der Oost et al., Eur. J. Biochem. 268:3062-3068 (2001)).

| Protein | Genbank ID | GI Number | Organism |
|---|---|---|---|
| mdh | AAC76268.1 | 1789632 | Escherichia coli |
| ldhA | NP_415898.1 | 16129341 | Escherichia coli |
| ldh | YP_725182.1 | 113866693 | Ralstonia eutropha |
| bdh | AAA58352.1 | 177198 | Homo sapiens |
| adh | AAA23199.2 | 60592974 | Clostridium beijerinckii NRRL B593 |
| adh | P14941.1 | 113443 | Thermoanaerobacter brockii HTD4 |
| sadh | CAD36475 | 21615553 | Rhodococcus ruber |
| adhA | AAC25556 | 3288810 | Pyrococcus furiosus |

A number of organisms encode genes that catalyze the reduction of 3-oxobutanol to 1,3-butanediol, including those belonging to the genus Bacillus, Brevibacterium, Candida, and Klebsiella among others, as described by Matsuyama et al. J Mol Cat B Enz, 11:513-521 (2001). One of these enzymes, SADH from Candida parapsilosis, was cloned and characterized in E. coli. A mutated Rhodococcus phenylacetaldehyde reductase (Sar268) and a Leifonia alcohol dehydrogenase have also been shown to catalyze this transformation at high yields (Itoh et al., Appl. Microbiol Biotechnol. 75:1249-1256 (2007)).

| Protein | Genbank ID | GI Number | Organism |
|---|---|---|---|
| sadh | BAA24528.1 | 2815409 | Candida parapsilosis |

Exemplary alcohol dehydrogenase enzymes include 3-oxoacyl-CoA reductase and acetoacetyl-CoA reductase. 3-Oxoacyl-CoA reductase enzymes (EC 1.1.1.35) convert 3-oxoacyl-CoA molecules into 3-hydroxyacyl-CoA molecules and are often involved in fatty acid beta-oxidation or phenylacetate catabolism. For example, subunits of two fatty acid oxidation complexes in E. coli, encoded by fadB and fadJ, function as 3-hydroxyacyl-CoA dehydrogenases (Binstock et al., Methods Enzymol. 71 Pt C:403-411 (1981)). Given the proximity in E. coli of paaH to other genes in the phenylacetate degradation operon (Nogales et al., 153:357-365 (2007)) and the fact that paaH mutants cannot grow on phenylacetate (Ismail et al., Eur. J Biochem. 270:3047-3054 (2003)), it is expected that the E. coli paaH gene also encodes a 3-hydroxyacyl-CoA dehydrogenase. Additional 3-oxoacyl-CoA enzymes include the gene products of phaC in Pseudomonas putida (Olivera et al., Proc. Natl. Acad. Sci U.S.A 95:6419-6424 (1998)) and paaC in Pseudomonas fluorescens (Di et al., 188:117-125 (2007)). These enzymes catalyze the reversible oxidation of 3-hydroxyadipyl-CoA to 3-oxoadipyl-CoA during the catabolism of phenylacetate or styrene.

Acetoacetyl-CoA reductase (EC 1.1.1.36) catalyzes the reduction of acetoacetyl-CoA to 3-hydroxybutyryl-CoA. This enzyme participates in the acetyl-CoA fermentation pathway to butyrate in several species of Clostridia and has been studied in detail (Jones et al., Microbiol Rev. 50:484-524 (1986)). Acetoacetyl-CoA reductase also participates in polyhydroxybutyrate biosynthesis in many organisms, and has also been used in metabolic engineering applications for overproducing PHB and 3-hydroxyisobutyrate (Liu et al., Appl. Microbiol. Biotechnol. 76:811-818 (2007); Qui et al., Appl. Microbiol. Biotechnol. 69:537-542 (2006)). The enzyme from Clostridium acetobutylicum, encoded by hbd, has been cloned and functionally expressed in E. coli (Youngleson et al., J Bacteriol. 171:6800-6807 (1989)). Additional gene candidates include phbB from Zoogloea ramigera (Ploux et al., Eur. J Biochem. 174:177-182 (1988)) and phaB from Rhodobacter sphaeroides (Alber et al., Mol. Microbiol 61:297-309 (2006)). The Z. ramigera gene is NADPH-dependent and the gene has been expressed in E. coli (Peoples et al., Mol. Microbiol 3:349-357 (1989)). Substrate specificity studies on the gene led to the conclusion that it could accept 3-oxopropionyl-CoA as a substrate besides acetoacetyl-CoA (Ploux et al., Eur. J Biochem. 174:177-182 (1988)). Additional genes include phaB in Paracoccus denitrificans, Hbd 1 (C-terminal domain) and Hbd2 (N-terminal domain) in Clostridium kluyveri (Hillmer and Gottschalk, Biochim. Biophys. Acta 3334:12-23 (1974)) and HSD17B10 in Bos taurus (Wakil et al., J Biol. Chem. 207:631-638 (1954)). The enzyme from Paracoccus denitrificans has been functionally expressed and characterized in E. coli (Yabutani et al., FEMS Microbiol Lett. 133:85-90 (1995)). A number of similar enzymes have been found in other species of Clostridia and in Metallosphaera sedula (Berg et al., Science. 318:1782-1786 (2007)). The enzyme from Candida tropicalis is a component of the peroxisomal fatty acid beta-oxidation multifunctional enzyme type 2 (MFE-2). The dehydrogenase B domain of this protein is catalytically active on acetoacetyl-CoA. The domain has been functionally expressed in E. coli, a crystal structure is available, and the catalytic mechanism is well-understood (Ylianttila et al., Biochem Biophys Res Commun 324:25-30 (2004); Ylianttila et al., J Mol Biol 358:1286-1295 (2006)).

| Protein | Genbank ID | GI Number | Organism |
|---|---|---|---|
| fadB | P21177.2 | 119811 | Escherichia coli |
| fadJ | P77399.1 | 3334437 | Escherichia coli |
| paaH | NP_415913.1 | 16129356 | Escherichia coli |
| Hbd2 | EDK34807.1 | 146348271 | Clostridium kluyveri |
| Hbd1 | EDK32512.1 | 146345976 | Clostridium kluyveri |
| phaC | NP_745425.1 | 26990000 | Pseudomonas putida |
| paaC | ABF82235.1 | 106636095 | Pseudomonas fluorescens |
| HSD17B10 | O02691.3 | 3183024 | Bos taurus |
| phbB | P23238.1 | 130017 | Zoogloea ramigera |
| phaB | YP_353825.1 | 77464321 | Rhodobacter sphaeroides |
| phaB | BAA08358 | 675524 | Paracoccus denitrificans |
| Hbd | NP_349314.1 | 15895965 | Clostridium acetobutylicum |
| Hbd | AAM14586.1 | 20162442 | Clostridium beijerinckii |
| Msed_1423 | YP_001191505 | 146304189 | Metallosphaera sedula |
| Msed_0399 | YP_001190500 | 146303184 | Metallosphaera sedula |
| Msed_0389 | YP_001190490 | 146303174 | Metallosphaera sedula |
| Msed_1993 | YP_001192057 | 146304741 | Metallosphaera sedula |
| Fox2 | Q02207 | 399508 | Candida tropicalis |

### 1.1.1.c Oxidoreductase (Acyl-CoA to Alcohol)

Bifunctional oxidoreductases convert an acyl-CoA to its corresponding alcohol. Enzymes with this activity can be used Steps K, O and W as depicted in FIG. 10.

Exemplary bifunctional oxidoreductases that convert an acyl-CoA to alcohol include those that transform substrates such as acetyl-CoA to ethanol (e.g., adhE from E. coli (Kessler et al., FEBS. Lett. 281:59-63 (1991))) and butyryl-CoA to butanol (e.g. adhE2 from C. acetobutylicum (Fontaine et al., J. Bacteriol. 184:821-830 (2002))). The C. acetobutylicum enzymes encoded by bdh I and bdh II (Walter, et al., J. Bacteriol. 174:7149-7158 (1992)), reduce acetyl-CoA and butyryl-CoA to ethanol and butanol, respectively. In addition to reducing acetyl-CoA to ethanol, the enzyme encoded by adhE in Leuconostoc mesenteroides has been shown to oxide the branched chain compound isobutyraldehyde to isobutyryl-CoA (Kazahaya et al., J. Gen. Appl. Microbiol. 18:43-55 (1972); Koo et al., Biotechnol Lett, 27:505-510 (2005)). Another exemplary enzyme can convert malonyl-CoA to 3-HP. An NADPH-dependent enzyme with this activity has characterized in Chloroflexus aurantiacus where it participates in the 3-hydroxypropionate cycle (Hugler et al., J Bacteriol, 184:2404-2410 (2002); Strauss et al., Eur J Biochem, 215:633-643 (1993)). This enzyme, with a mass of 300 kDa, is highly substrate-specific and shows little sequence similarity to other known oxidoreductases (Hugler et al., supra). No enzymes in other organisms have been shown to catalyze this specific reaction; however there is bioinformatic evidence that other organisms may have similar pathways (Klatt et al., Env Microbiol, 9:2067-2078 (2007)). Enzyme candidates in other organisms including Roseiflexus castenholzii, Erythrobacter sp. NAP1 and marine gamma proteobacterium HTCC2080 can be inferred by sequence similarity.

| Protein | GenBank ID | GI Number | Organism |
|---|---|---|---|
| adhE | NP_415757.1 | 16129202 | Escherichia coli |
| adhE2 | AAK09379.1 | 12958626 | Clostridium acetobutylicum |
| bdh I | NP_349892.1 | 15896543 | Clostridium acetobutylicum |
| bdh II | NP_349891.1 | 15896542 | Clostridium acetobutylicum |
| adhE | AAV66076.1 | 55818563 | Leuconostoc mesenteroides |
| mcr | AAS20429.1 | 42561982 | Chloroflexus aurantiacus |
| Rcas2929 | YP_001433009.1 | 156742880 | Roseiflexus castenholzii |
| NAP1_02720 | ZP_01039179.1 | 85708113 | Erythrobacter sp. NAP1 |
| MGP2080_00535 | ZP_01626393.1 | 119504313 | marine gamma proteobacterium HTCC2080 |

Longer chain acyl-CoA molecules can be reduced to their corresponding alcohols by enzymes such as the jojoba (Simmondsia chinensis) FAR which encodes an alcohol-forming fatty acyl-CoA reductase. Its overexpression in E. coli resulted in FAR activity and the accumulation of fatty alcohol (Metz et al., Plant Physiol, 122:635-644 (2000)).

| Protein | GenBank ID | GI Number | Organism |
|---|---|---|---|
| FAR | AAD38039.1 | 5020215 | Simmondsia chinensis |

Another candidate for catalyzing these steps is 3-hydroxy-3-methylglutaryl-CoA reductase (or HMG-CoA reductase). This enzyme naturally reduces the CoA group in 3-hydroxy-3-methylglutaryl-CoA to an alcohol forming mevalonate. The hmgA gene of Sulfolobus solfataricus, encoding 3-hydroxy-3-methylglutaryl-CoA reductase, has been cloned, sequenced, and expressed in E. coli (Bochar et al., J Bacteriol. 179:3632-3638 (1997)). S. cerevisiae also has two HMG-CoA reductases in it (Basson et al., Proc. Natl. Acad. Sci. U.S.A 83:5563-5567 (1986)). The gene has also been isolated from Arabidopsis thaliana and has been shown to complement the HMG-COA reductase activity in S. cerevisiae (Learned et al., Proc. Natl. Acad. Sci. U.S.A 86:2779-2783 (1989)).

| Protein | GenBank ID | GI Number | Organism |
|---|---|---|---|
| HMG1 | CAA86503.1 | 587536 | Saccharomyces cerevisiae |
| HMG2 | NP_013555 | 6323483 | Saccharomyces cerevisiae |
| HMG1 | CAA70691.1 | 1694976 | Arabidopsis thaliana |
| hmgA | AAC45370.1 | 2130564 | Sulfolobus solfataricus |

### 1.2.1.b Oxidoreductase (Acyl-CoA to Aldehyde)

Acyl-CoA reductases in the 1.2.1 family reduce an acyl-CoA to its corresponding aldehyde. Such a conversion is utilized in Steps I, N and V of FIG. 10. Several acyl-CoA reductase enzymes have been described in the open literature and represent suitable candidates for this step. These are described below.

Acyl-CoA reductases or acylating aldehyde dehydrogenases reduce an acyl-CoA to its corresponding aldehyde. Exemplary enzymes include fatty acyl-CoA reductase, succinyl-CoA reductase (EC 1.2.1.76), acetyl-CoA reductase, butyryl-CoA reductase and propionyl-CoA reductase (EC 1.2.1.3). Exemplary fatty acyl-CoA reductases enzymes are encoded by acr1 of Acinetobacter calcoaceticus (Reiser, Journal of Bacteriology 179:2969-2975 (1997)) and Acinetobacter sp. M-1 (Ishige et al., Appl. Environ. Microbiol. 68:1192-1195 (2002)). Enzymes with succinyl-CoA reductase activity are encoded by sucD of Clostridium kluyveri (Sohling, J. Bacteriol. 178:871-880 (1996)) and sucD of P. gingivalis (Takahashi, J. Bacteriol 182:4704-4710 (2000)). Additional succinyl-CoA reductase enzymes participate in the 3-hydroxypropionate/4-hydroxybutyrate cycle of thermophilic archaea including Metallosphaera sedula (Berg et al., Science 318:1782-1786 (2007)) and Thermoproteus neutrophilus (Ramos-Vera et al., J Bacteriol., 191:4286-4297 (2009)). The M. sedula enzyme, encoded by Msed_{—}0709, is strictly NADPH-dependent and also has malonyl-CoA reductase activity. The T. neutrophilus enzyme is active with both NADPH and NADH. The enzyme acylating acetaldehyde dehydrogenase in Pseudomonas sp, encoded by bphG, is yet another as it has been demonstrated to oxidize and acylate acetaldehyde, propionaldehyde, butyraldehyde, isobutyraldehyde and formaldehyde (Powlowski, J. Bacteriol. 175:377-385 (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Kazahaya, J. Gen. Appl. Microbiol. 18:43-55 (1972); and Koo et al., Biotechnol Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci Biotechnol Biochem., 71:58-68 (2007)). Exemplary propionyl-CoA reductase enzymes include pduP of Salmonella typhimurium LT2 (Leal, Arch. Microbiol. 180:353-361 (2003)) and eutE from E. coli (Skraly, WO Patent No. 2004/024876). The propionyl-CoA reductase of Salmonella typhimurium LT2, which naturally converts propionyl-CoA to propionaldehyde, also catalyzes the reduction of 5-hydroxyvaleryl-CoA to 5-hydroxypentanal (WO 2010/068953A2).

| Protein | GenBank ID | GI Number | Organism |
|---|---|---|---|
| acr1 | YP_047869.1 | 50086359 | Acinetobacter calcoaceticus |
| acr1 | AAC45217 | 1684886 | Acinetobacter baylyi |
| acr1 | BAB85476.1 | 18857901 | Acinetobacter sp. Strain M-1 |
| MSED_0709 | YP_001190808.1 | 146303492 | Metallosphaera sedula |
| Tneu_0421 | ACB39369.1 | 170934108 | Thermoproteus neutrophilus |
| sucD | P38947.1 | 172046062 | Clostridium kluyveri |
| sucD | NP_904963.1 | 34540484 | Porphyromonas gingivalis |
| bphG | BAA03892.1 | 425213 | Pseudomonas sp |
| adhE | AAV66076.1 | 55818563 | Leuconostoc mesenteroides |
| bld | AAP42563.1 | 31075383 | Clostridium |
| | | | saccharoperbutylacetonicum |
| pduP | NP_460996 | 16765381 | Salmonella typhimurium LT2 |
| eutE | NP_416950 | 16130380 | Escherichia coli |

An additional enzyme that converts an acyl-CoA to its corresponding aldehyde is malonyl-CoA reductase which transforms malonyl-CoA to malonic semialdehyde. Malonyl-CoA reductase is a key enzyme in autotrophic carbon fixation via the 3-hydroxypropionate cycle in thermoacidophilic archaeal bacteria (Berg, Science 318:1782-1786 (2007); and Thauer, Science 318:1732-1733 (2007)). The enzyme utilizes NADPH as a cofactor and has been characterized in Metallosphaera and Sulfolobus sp. (Alber et al., J. Bacteriol. 188:8551-8559 (2006); and Hugler, J. Bacteriol. 184:2404-2410 (2002)). The enzyme is encoded by Msed_{—}0709 in Metallosphaera sedula (Alber et al., J. Bacteriol. 188:8551-8559 (2006); and Berg, Science 318:1782-1786 (2007)). A gene encoding a malonyl-CoA reductase from Sulfolobus tokodaii was cloned and heterologously expressed in E. coli (Alber et al., J Bacteriol 188:8551-8559 (2006). This enzyme has also been shown to catalyze the conversion of methylmalonyl-CoA to its corresponding aldehyde (WO2007141208 (2007)). Although the aldehyde dehydrogenase functionality of these enzymes is similar to the bifunctional dehydrogenase from Chloroflexus aurantiacus, there is little sequence similarity. Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates can be found by sequence homology to proteins in other organisms including Sulfolobus solfataricus and Sulfolobus acidocaldarius and have been listed below. Yet another candidate for CoA-acylating aldehyde dehydrogenase is the ald gene from Clostridium beijerinckii (Toth, Appl. Environ. Microbiol. 65:4973-4980 (1999). This enzyme has been reported to reduce acetyl-CoA and butyryl-CoA to their corresponding aldehydes. This gene is very similar to eutE that encodes acetaldehyde dehydrogenase of Salmonella typhimurium and E. coli (Toth, Appl. Environ. Microbiol. 65:4973-4980 (1999).

| Protein | GenBank ID | GI Number | Organism |
|---|---|---|---|
| Msed_0709 | YP_001190808.1 | 146303492 | Metallosphaera sedula |
| Mcr | NP_378167.1 | 15922498 | Sulfolobus tokodaii |
| asd-2 | NP_343563.1 | 15898958 | Sulfolobus solfataricus |
| Saci_2370 | YP_256941.1 | 70608071 | Sulfolobus acidocaldarius |
| Ald | AAT66436 | 49473535 | Clostridium beijerinckii |
| eutE | AAA80209 | 687645 | Salmonella typhimurium |
| eutE | P77445 | 2498347 | Escherichia coli |

### 1.2.1.e Oxidoreductase (Acid to Aldehyde)

The conversion of an acid to an aldehyde is thermodynamically unfavorable and typically requires energy-rich cofactors and multiple enzymatic steps. Direct conversion of the acid to aldehyde by a single enzyme is catalyzed by an acid reductase enzyme in the 1.2.1 family. An enzyme in this EC class can be used in Steps F, Z and AG of FIG. 10.

Exemplary acid reductase enzymes include carboxylic acid reductase, alpha-aminoadipate reductase and retinoic acid reductase. Carboxylic acid reductase (CAR), found in Nocardia iowensis, catalyzes the magnesium, ATP and NADPH-dependent reduction of carboxylic acids to their corresponding aldehydes (Venkitasubramanian et al., J Biol. Chem. 282:478-485 (2007)). The natural substrate of this enzyme is benzoate and the enzyme exhibits broad acceptance of aromatic substrates including p-toluate (Venkitasubramanian et al., Biocatalysis in Pharmaceutical and Biotechnology Industries. CRC press (2006)). The enzyme from Nocardia iowensis, encoded by car, was cloned and functionally expressed in E. coli (Venkitasubramanian et al., J Biol. Chem. 282:478-485 (2007)). CAR requires post-translational activation by a phosphopantetheine transferase (PPTase) that converts the inactive apo-enzyme to the active holo-enzyme (Hansen et al., Appl. Environ. Microbiol 75:2765-2774 (2009)). Expression of the npt gene, encoding a specific PPTase, product improved activity of the enzyme. An additional enzyme candidate found in Streptomyces griseus is encoded by the griC and griD genes. This enzyme is believed to convert 3-amino-4-hydroxybenzoic acid to 3-amino-4-hydroxybenzaldehyde as deletion of either griC or griD led to accumulation of extracellular 3-acetylamino-4-hydroxybenzoic acid, a shunt product of 3-amino-4-hydroxybenzoic acid metabolism (Suzuki, et al., J Antibiot. 60(6):380-387 (2007)). Co-expression of griC and griD with SGR_{—}665, an enzyme similar in sequence to the Nocardia iowensis npt, can be beneficial.

| Gene | GenBank Accession No. | GI No. | Organism |
|---|---|---|---|
| car | AAR91681.1 | 40796035 | Nocardia iowensis |
| npt | ABI83656.1 | 114848891 | Nocardia iowensis |
| griC | YP_001825755.1 | 182438036 | Streptomyces griseus |
| griD | YP_001825756.1 | 182438037 | Streptomyces griseus |

Additional car and npt genes can be identified based on sequence homology.

| Gene name | GI No. | GenBank Accession No. | Organism |
|---|---|---|---|
| fadD9 | 121638475 | YP_978699.1 | Mycobacterium bovis BCG |
| BCG_2812c | 121638674 | YP_978898.1 | Mycobacterium bovis BCG |
| nfa20150 | 54023983 | YP_118225.1 | Nocardia farcinica IFM 10152 |
| nfa40540 | 54026024 | YP_120266.1 | Nocardia farcinica IFM 10152 |
| | 182440583 | YP_001828302.1 | Streptomyces griseus subsp. griseus |
| | | | NBRC 13350 |
| | 182434458 | YP_001822177.1 | Streptomyces griseus subsp. griseus |
| | | | NBRC 13350 |
| MSMEG_2956 | YP_887275.1 | YP_887275.1 | Mycobacterium smegmatis MC2 155 |
| MSMEG_5739 | YP_889972.1 | 118469671 | Mycobacterium smegmatis MC2 155 |
| MSMEG_2648 | YP_886985.1 | 118471293 | Mycobacterium smegmatis MC2 155 |
| MAP1040c | NP_959974.1 | 41407138 | Mycobacterium avium subsp. |
| | | | paratuberculosis K-10 |
| MAP2899c | NP_961833.1 | 41408997 | Mycobacterium avium subsp. |
| | | | paratuberculosis K-10 |
| MMAR_2117 | YP_001850422.1 | 183982131 | Mycobacterium marinum M |
| MMAR_2936 | YP_001851230.1 | 183982939 | Mycobacterium marinum M |
| MMAR_1916 | YP_001850220.1 | 183981929 | Mycobacterium marinum M |
| TpauDRAFT_ | ZP_04027864.1 | 227980601 | Tsukamurella paurometabola DSM |
| 33060 | | | 20162 |
| TpauDRAFT_ | ZP_04026660.1 | ZP_04026660.1 | Tsukamurella paurometabola DSM |
| 20920 | | | 20162 |
| CPCC7001_1320 | ZP_05045132.1 | 254431429 | Cyanobium PCC7001 |
| DDBDRAFT_ | XP_636931.1 | 66806417 | Dictyostelium discoideum AX4 |
| 0187729 | | | |

An enzyme with similar characteristics, alpha-aminoadipate reductase (AAR, EC 1.2.1.31), participates in lysine biosynthesis pathways in some fungal species. This enzyme naturally reduces alpha-aminoadipate to alpha-aminoadipate semialdehyde. The carboxyl group is first activated through the ATP-dependent formation of an adenylate that is then reduced by NAD(P)H to yield the aldehyde and AMP. Like CAR, this enzyme utilizes magnesium and requires activation by a PPTase. Enzyme candidates for AAR and its corresponding PPTase are found in Saccharomyces cerevisiae (Morris et al., Gene 98:141-145 (1991)), Candida albicans (Guo et al., Mol. Genet. Genomics 269:271-279 (2003)), and Schizosaccharomyces pombe (Ford et al., Curr. Genet. 28:131-137 (1995)). The AAR from S. pombe exhibited significant activity when expressed in E. coli (Guo et al., Yeast 21:1279-1288 (2004)). The AAR from Penicillium chrysogenum accepts S-carboxymethyl-L-cysteine as an alternate substrate, but did not react with adipate, L-glutamate or diaminopimelate (Hijarrubia et al., J Biol. Chem. 278:8250-8256 (2003)). The gene encoding the P. chrysogenum PPTase has not been identified to date and no high-confidence hits were identified by sequence comparison homology searching

| Gene | GenBank Accession No. | GI No. | Organism |
|---|---|---|---|
| LYS2 | AAA34747.1 | 171867 | Saccharomyces cerevisiae |
| LYS5 | P50113.1 | 1708896 | Saccharomyces cerevisiae |
| LYS2 | AAC02241.1 | 2853226 | Candida albicans |
| LYS5 | AAO26020.1 | 28136195 | Candida albicans |
| Lys1p | P40976.3 | 13124791 | Schizosaccharomyces pombe |
| Lys7p | Q10474.1 | 1723561 | Schizosaccharomyces pombe |
| Lys2 | CAA74300.1 | 3282044 | Penicillium chrysogenum |

### 1.2.1.f Oxidoreductase (Acyl-ACP to Aldehyde)

The reduction of an acyl-ACP to its corresponding aldehyde is catalyzed by an acyl-ACP reductase (AAR). Such a transformation is depicted in steps J, M and U of FIG. 10. Suitable enzyme candidates include the orf1594 gene product of Synechococcus elongatus PCC7942 and homologs thereof (Schirmer et al, Science, 329: 559-62 (2010)). The S. elongates PCC7942 acyl-ACP reductase is coexpressed with an aldehyde decarbonylase in an operon that appears to be conserved in a majority of cyanobacterial organisms. This enzyme, expressed in E. coli together with the aldehyde decarbonylase, conferred the ability to produce alkanes. The P. marinus AAR was also cloned into E. coli and, together with a decarbonylase, demonstrated to produce alkanes (US Application 2011/0207203).

| Protein | GenBank ID | GI Number | Organism |
|---|---|---|---|
| orf1594 | YP_400611.1 | 81300403 | Synechococcus elongatus |
| | | | PCC7942 |
| PMT9312_0533 | YP_397030.1 | 78778918 | Prochlorococcus marinus |
| | | | MIT 9312 |
| syc0051_d | YP_170761.1 | 56750060 | Synechococcus elongatus |
| | | | PCC 6301 |
| Ava_2534 | YP_323044.1 | 75908748 | Anabaena variabilis |
| | | | ATCC 29413 |
| alr5284 | NP_489324.1 | 17232776 | Nostoc sp. PCC 7120 |
| Aazo_3370 | YP_003722151.1 | 298491974 | Nostoc azollae |
| Cyan7425_0399 | YP_002481152.1 | 220905841 | Cyanothece sp. PCC 7425 |
| N9414_21225 | ZP_01628095.1 | 119508943 | Nodularia spumigena |
| | | | CCY9414 |
| L8106_07064 | ZP_01619574.1 | 119485189 | Lyngbya sp. PCC 8106 |

### 2.3.1.e Acyl-ACP C-Acyltransferase (Decarboxylating)

In step A of FIG. 10, acetoacetyl-ACP is formed from malonyl-ACP and either acetyl-CoA or acetyl-ACP. This reaction is catalyzed by an acyl-ACP C-acyltransferase in EC class 2.3.1. The condensation of malonyl-ACP and acetyl-CoA is catalyzed by beta-ketoacyl-ACP synthase (KAS, EC 2.3.1.180). E. coli has three KAS enzymes encoded by fabB, fabF and fabH. FabH (KAS III), the key enzyme of initiation of fatty acid biosynthesis in E. coli, is selective for the formation of acetoacetyl-ACP. FabB and FabF catalyze the condensation of malonyl-ACP with acyl-ACP substrates and function primarily in fatty acid elongation although they can also react with acetyl-ACP and thereby participate in fatty acid initiation. For example, the Bacillus subtilis KAS enzymes are similar to FabH but are less selective, accepting branched acyl-CoA substrates (Choi et al, J Bacteriol 182:365-70 (2000)).

| Protein | GenBank ID | GI Number | Organism |
|---|---|---|---|
| fabB | AAC75383.1 | 1788663 | Escherichia coli |
| fabF | AAC74179.1 | 1787337 | Escherichia coli |
| fabH | AAC74175.1 | 1787333 | Escherichia coli |
| FabHA | NP_389015.1 | 16078198 | Bacillus subtilis |
| FabHB | NP_388898.1 | 16078081 | Bacillus subtilis |

Alternately, acetyl-CoA can first be activated to acetyl-ACP and subsequently condensed to acetoacetyl-ACP by two enzymes, acetyl-CoA:ACP transacylase (EC 2.3.1.38) and acetoacetyl-ACP synthase (EC 2.3.1.41). Acetyl-CoA:ACP transacylase converts acetyl-CoA and an acyl carrier protein to acetyl-ACP, releasing CoA. Enzyme candidates for acetyl-CoA:ACP transacylase are described in section EC 2.3.1.f below. Acetoacetyl-ACP synthase enzymes catalyze the condensation of acetyl-ACP and malonyl-ACP. This activity is catalyzed by FabF and FabB of E. coli, as well as the multifunctional eukaryotic fatty acid synthase enzyme complexes described in EC 2.3.1.g.

### 2.3.1.f CoA-ACP Acyltransferase

The exchange of an ACP moiety for a CoA is catalyzed by enzymes in EC class 2.3.1. This reaction is shown in steps D, X, and AE of FIG. 10. Activation of acetyl-CoA to acetyl-ACP (step A of FIG. 10) is also catalyzed by a CoA:ACP acyltransferase. Enzymes with CoA-ACP acyltransferase activity include acetyl-CoA:ACP transacylase (EC 2.3.1.38) and malonyl-CoA:ACP transacylase (EC 2.3.1.39).

The FabH (KASIII) enzyme of E. coli functions as an acyl-CoA:ACP transacylase, in addition to its primary activity of forming acetoacetyl-ACP. Butyryl-ACP is accepted as an alternate substrate of FabH (Prescott et al, Adv. Enzymol. Relat. Areas Mol, 36:269-311 (1972)). Acetyl-CoA:ACP transacylase enzymes from Plasmodium falciparum and Streptomyces avermitillis have been heterologously expressed in E. coli (Lobo et al, Biochem 40:11955-64 (2001)). A synthetic KASIII (FabH) from P. falciparum expressed in a fabH-deficient Lactococcus lactis host was able to complement the native fadH activity (Du et al, AEM 76:3959-66 (2010)). The acetyl-CoA:ACP transacylase enzyme from Spinacia oleracea accepts other acyl-ACP molecules as substrates, including butyryl-ACP (Shimakata et al, Methods Enzym 122:53-9 (1986)). The sequence of this enzyme has not been determined to date. Malonyl-CoA:ACP transacylase enzymes include FabD of E. coli and Brassica napsus (Verwoert et al, J Bacteriol, 174:2851-7 (1992); Simon et al, FEBS Left 435:204-6 (1998)). FabD of B. napsus was able to complement fabD-deficient E. coli. The multifunctional eukaryotic fatty acid synthase enzyme complexes (described in EC 2.3.1.g) also catalyze this activity.

| Protein | GenBank ID | GI Number | Organism |
|---|---|---|---|
| fabH | AAC74175.1 | 1787333 | Escherichia coli |
| fadA | NP_824032.1 | 29829398 | Streptomyces avermitillis |
| fabH | AAC63960.1 | 3746429 | Plasmodium falciparum |
| Synthetic construct | ACX34097.1 | 260178848 | Plasmodium falciparum |
| fabH | CAL98359.1 | 124493385 | Lactococcus lactis |
| fabD | AAC74176.1 | 1787334 | Escherichia coli |
| fabD | CAB45522.1 | 5139348 | Brassica napsus |

### 2.3.1.2 Fatty Acid Synthase

Steps A, B, and C of FIG. 10 can together be catalyzed fatty acid synthase or fatty-acyl-CoA synthase, multifunctional enzyme complexes composed of multiple copies of one or more subunits. The fatty acid synthase of Saccharomyces cerevisiae is a dodecamer composed of two multifunctional subunits FAS1 and FAS2 that together catalyze all the reactions required for fatty acid synthesis: activation, priming, elongation and termination (Lomakin et al, Cell 129:319-32 (2007)). This enzyme complex catalyzes the formation of long chain fatty acids from acetyl-CoA and malonyl-CoA. The favored product of eukaryotic FAS systems is palmitic acid (C16) Similar fatty acid synthase complexes are found in Candida parapsilosis and Thermomyces lanuginosus (Nguyen et al, PLoS One 22:e8421 (2009); Jenni et al, Science 316:254-61 (2007)). The multifunctional Fas enzymes of Mycobacterium tuberculosis and mammals such as Homo sapiens are also suitable candidates (Fernandes and Kolattukudy, Gene 170:95-99 (1996) and Smith et al, Prog Lipid Res 42:289-317 (2003)).

| Protein | GenBank ID | GI Number | Organism |
|---|---|---|---|
| FAS1 | CAA82025.1 | 486321 | Saccharomyces cerevisiae |
| FAS2 | CAA97948.1 | 1370478 | Saccharomyces cerevisiae |
| Fas1 | ABO37973.1 | 133751597 | Thermomyces lanuginosus |
| Fas2 | ABO37974.1 | 133751599 | Thermomyces lanuginosus |
| Fas | AAB03809.1 | 1036835 | Mycobacterium tuberculosis |
| Fas | NP_004095.4 | 41872631 | Homo sapiens |

### 2.8.3.a CoA Transferase

Enzymes in the 2.8.3 family catalyze the reversible transfer of a CoA moiety from one molecule to another. Such a transformation can be utilized for Steps E, Y and AF of FIG. 10. Several CoA transferase enzymes have been described in the open literature and represent suitable candidates for these steps. These are described below.

Many transferases have broad specificity and thus can utilize CoA acceptors as diverse as acetate, succinate, propionate, butyrate, 2-methylacetoacetate, 3-ketohexanoate, 3-ketopentanoate, valerate, crotonate, 3-mercaptopropionate, propionate, vinylacetate, butyrate, among others. For example, an enzyme from Roseburia sp. A2-183 was shown to have butyryl-CoA:acetate:CoA transferase and propionyl-CoA:acetate:CoA transferase activity (Charrier et al., Microbiology 152, 179-185 (2006)). Close homologs can be found in, for example, Roseburia intestinalis L1-82, Roseburia inulinivorans DSM 16841, Eubacterium rectale ATCC 33656. Another enzyme with propionyl-CoA transferase activity can be found in Clostridium propionicum (Selmer et al., Eur J Biochem 269, 372-380 (2002)). This enzyme can use acetate, (R)-lactate, (S)-lactate, acrylate, and butyrate as the CoA acceptor (Selmer et al., Eur J Biochem 269, 372-380 (2002); Schweiger and Buckel, FEBS Letters, 171(1) 79-84 (1984)). Close homologs can be found in, for example, Clostridium novyi NT, Clostridium beijerinckii NCIMB 8052, and Clostridium botulinum C str. Eklund. YgfH encodes a propionyl CoA:succinate CoA transferase in E. coli (Haller et al., Biochemistry, 39(16) 4622-4629). Close homologs can be found in, for example, Citrobacter youngae ATCC 29220, Salmonella enterica subsp. arizonae serovar, and Yersinia intermedia ATCC 29909. These proteins are identified below.

| Protein | GenBank ID | GI Number | Organism |
|---|---|---|---|
| Ach1 | AAX19660.1 | 60396828 | Roseburia sp. A2-183 |
| ROSINTL182_07121 | ZP_04743841.2 | 257413684 | Roseburia intestinalis L1-82 |
| ROSEINA2194_03642 | ZP_03755203.1 | 225377982 | Roseburia inulinivorans |
| EUBREC_3075 | YP_002938937.1 | 238925420 | Eubacterium rectale ATCC 33656 |
| Pct | CAB77207.1 | 7242549 | Clostridium propionicum |
| NT01CX_2372 | YP_878445.1 | 118444712 | Clostridium novyi NT |
| Cbei_4543 | YP_001311608.1 | 150019354 | Clostridium beijerinckii |
| CBC_A0889 | ZP_02621218.1 | 168186583 | Clostridium botulinum C str. Eklund |
| ygfH | NP_417395.1 | 16130821 | Escherichia coli |
| CIT292_04485 | ZP_03838384.1 | 227334728 | Citrobacter youngae ATCC 29220 |
| SARI_04582 | YP_001573497.1 | 161506385 | Salmonella enterica subsp. arizonae serovar |
| yinte0001_14430 | ZP_04635364.1 | 238791727 | Yersinia intermedia ATCC 29909 |

An additional candidate enzyme is the two-unit enzyme encoded by pcaI and pcaJ in Pseudomonas, which has been shown to have 3-oxoadipyl-CoA/succinate transferase activity (Kaschabek et al., supra). Similar enzymes based on homology exist in Acinetobacter sp. ADP1 (Kowalchuk et al., Gene 146:23-30 (1994)) and Streptomyces coelicolor. Additional exemplary succinyl-CoA:3:oxoacid-CoA transferases are present in Helicobacter pylori (Corthesy-Theulaz et al., J. Biol. Chem. 272:25659-25667 (1997)) and Bacillus subtilis (Stols et al., Protein. Expr. Purif. 53:396-403 (2007)). These proteins are identified below.

| Protein | GenBank ID | GI Number | Organism |
|---|---|---|---|
| pcaI | AAN69545.1 | 24985644 | Pseudomonas putida |
| pcaJ | NP_746082.1 | 26990657 | Pseudomonas putida |
| pcaI | YP_046368.1 | 50084858 | Acinetobacter sp. ADP1 |
| pcaJ | AAC37147.1 | 141776 | Acinetobacter sp. ADP1 |
| pcaI | NP_630776.1 | 21224997 | Streptomyces coelicolor |
| pcaJ | NP_630775.1 | 21224996 | Streptomyces coelicolor |
| HPAG1_0676 | YP_627417 | 108563101 | Helicobacter pylori |
| HPAG1_0677 | YP_627418 | 108563102 | Helicobacter pylori |
| ScoA | NP_391778 | 16080950 | Bacillus subtilis |
| ScoB | NP_391777 | 16080949 | Bacillus subtilis |

A CoA transferase that can utilize acetate as the CoA acceptor is acetoacetyl-CoA transferase, encoded by the E. coli atoA (alpha subunit) and atoD (beta subunit) genes (Vanderwinkel et al., Biochem. Biophys. Res Commun. 33:902-908 (1968); Korolev et al., Acta Crystallogr. D Biol Crystallogr. 58:2116-2121 (2002)). This enzyme has also been shown to transfer the CoA moiety to acetate from a variety of branched and linear acyl-CoA substrates, including isobutyrate (Matthies et al., Appl Environ Microbiol 58:1435-1439 (1992)), valerate (Vanderwinkel et al., supra) and butanoate (Vanderwinkel et al., supra). Similar enzymes exist in Corynebacterium glutamicum ATCC 13032 (Duncan et al., Appl Environ Microbiol 68:5186-5190 (2002)), Clostridium acetobutylicum (Cary et al., Appl Environ Microbiol 56:1576-1583 (1990)), and Clostridium saccharoperbutylacetonicum (Kosaka et al., Biosci. Biotechnol Biochem. 71:58-68 (2007)). These proteins are identified below.

| Protein | GenBank ID | GI Number | Organism |
|---|---|---|---|
| atoA | P76459.1 | 2492994 | Escherichia coli K12 |
| atoD | P76458.1 | 2492990 | Escherichia coli K12 |
| actA | YP_226809.1 | 62391407 | Corynebacterium glutamicum |
| | | | ATCC 13032 |
| cg0592 | YP_224801.1 | 62389399 | Corynebacterium glutamicum |
| | | | ATCC 13032 |
| ctfA | NP_149326.1 | 15004866 | Clostridium acetobutylicum |
| ctfB | NP_149327.1 | 15004867 | Clostridium acetobutylicum |
| ctfA | AAP42564.1 | 31075384 | Clostridium |
| | | | saccharoperbutylacetonicum |
| ctfB | AAP42565.1 | 31075385 | Clostridium |
| | | | saccharoperbutylacetonicum |

Additional exemplary transferase candidates are catalyzed by the gene products of cat 1, cat2, and cat3 of Clostridium kluyveri which have been shown to exhibit succinyl-CoA, 4-hydroxybutyryl-CoA, and butyryl-CoA transferase activity, respectively (Seedorf et al., supra; Sohling et al., Eur. J Biochem. 212:121-127 (1993); Sohling et al., J Bacteriol. 178:871-880 (1996)) Similar CoA transferase activities are also present in Trichomonas vaginalis (van Grinsven et al., J. Biol. Chem. 283:1411-1418 (2008)) and Trypanosoma brucei (Riviere et al., J. Biol. Chem. 279:45337-45346 (2004)). These proteins are identified below.

| Protein | GenBank ID | GI Number | Organism |
|---|---|---|---|
| cat1 | P38946.1 | 729048 | Clostridium kluyveri |
| cat2 | P38942.2 | 172046066 | Clostridium kluyveri |
| cat3 | EDK35586.1 | 146349050 | Clostridium kluyveri |
| TVAG_395550 | XP_001330176 | 123975034 | Trichomonas vaginalis G3 |
| Tb11.02.0290 | XP_828352 | 71754875 | Trypanosoma brucei |

The glutaconate-CoA-transferase (EC 2.8.3.12) enzyme from anaerobic bacterium Acidaminococcus fermentans reacts with diacid glutaconyl-CoA and 3-butenoyl-CoA (Mack et al., FEBS Lett. 405:209-212 (1997)). The genes encoding this enzyme are gctA and gctB. This enzyme has reduced but detectable activity with other CoA derivatives including glutaryl-CoA, 2-hydroxyglutaryl-CoA, adipyl-CoA and acrylyl-CoA (Buckel et al., Eur. J. Biochem. 118:315-321 (1981)). The enzyme has been cloned and expressed in E. coli (Mack et al., Eur. J. Biochem. 226:41-51 (1994)). These proteins are identified below.

| Protein | GenBank ID | GI Number | Organism |
|---|---|---|---|
| gctA | CAA57199.1 | 559392 | Acidaminococcus fermentans |
| gctB | CAA57200.1 | 559393 | Acidaminococcus fermentans |

### 3.1.2.a CoA Hydrolase

Enzymes in the 3.1.2 family hydrolyze acyl-CoA molecules to their corresponding acids. Such a transformation can be utilized in Steps E, Y and AF of FIG. 10. Several such enzymes have been described in the literature and represent suitable candidates for these steps.

For example, the enzyme encoded by acot12 from Rattus norvegicus brain (Robinson et al., Biochem. Biophys. Res. Commun. 71:959-965 (1976)) can react with butyryl-CoA, hexanoyl-CoA and malonyl-CoA. The human dicarboxylic acid thioesterase, encoded by acot8, exhibits activity on glutaryl-CoA, adipyl-CoA, suberyl-CoA, sebacyl-CoA, and dodecanedioyl-CoA (Westin et al., J. Biol. Chem. 280:38125-38132 (2005)). The closest E. coli homolog to this enzyme, tesB, can also hydrolyze a range of CoA thiolesters (Naggert et al., J Biol Chem 266:11044-11050 (1991)). A similar enzyme has also been characterized in the rat liver (Deana R., Biochem Int 26:767-773 (1992)). Additional enzymes with hydrolase activity in E. coli include ybgC, paaI, and ybdB (Kuznetsova, et al., FEMS Microbiol Rev, 2005, 29(2):263-279; Song et al., J Biol Chem, 2006, 281(16):11028-38). Though its sequence has not been reported, the enzyme from the mitochondrion of the pea leaf has a broad substrate specificity, with demonstrated activity on acetyl-CoA, propionyl-CoA, butyryl-CoA, palmitoyl-CoA, oleoyl-CoA, succinyl-CoA, and crotonyl-CoA (Zeiher et al., Plant. Physiol. 94:20-27 (1990)) The acetyl-CoA hydrolase, ACH1, from S. cerevisiae represents another candidate hydrolase (Buu et al., J. Biol. Chem. 278:17203-17209 (2003)).

| Protein | GenBank Accession No. | GI Number | Organism |
|---|---|---|---|
| acot12 | NP_570103.1 | 18543355 | Rattus norvegicus |
| tesB | NP_414986 | 16128437 | Escherichia coli |
| acot8 | CAA15502 | 3191970 | Homo sapiens |
| acot8 | NP_570112 | 51036669 | Rattus norvegicus |
| tesA | NP_415027 | 16128478 | Escherichia coli |
| ybgC | NP_415264 | 16128711 | Escherichia coli |
| paaI | NP_415914 | 16129357 | Escherichia coli |
| ybdB | NP_415129 | 16128580 | Escherichia coli |
| ACH1 | NP_009538 | 6319456 | Saccharomyces cerevisiae |

Additional hydrolase enzymes include 3-hydroxyisobutyryl-CoA hydrolase which has been described to efficiently catalyze the conversion of 3-hydroxyisobutyryl-CoA to 3-hydroxyisobutyrate during valine degradation (Shimomura et al., J Biol Chem. 269:14248-14253 (1994)). Genes encoding this enzyme include hibch of Rattus norvegicus (Shimomura et al., Methods Enzymol. 324:229-240 (2000)) and Homo sapiens (Shimomura et al., supra). Similar gene candidates can also be identified by sequence homology, including hibch of Saccharomyces cerevisiae and BC_{—}2292 of Bacillus cereus.

| Protein | GenBank No. | GI Number | Organism |
|---|---|---|---|
| hibch | Q5XIE6.2 | 146324906 | Rattus norvegicus |
| hibch | Q6NVY1.2 | 146324905 | Homo sapiens |
| hibch | P28817.2 | 2506374 | Saccharomyces cerevisiae |
| BC_2292 | AP09256 | 29895975 | Bacillus cereus |

Yet another candidate hydrolase is the glutaconate CoA-transferase from Acidaminococcus fermentans. This enzyme was transformed by site-directed mutagenesis into an acyl-CoA hydrolase with activity on glutaryl-CoA, acetyl-CoA and 3-butenoyl-CoA (Mack et al., FEBS. Lett. 405:209-212 (1997)). This suggests that the enzymes encoding succinyl-CoA:3-ketoacid-CoA transferases and acetoacetyl-CoA:acetyl-CoA transferases may also serve as candidates for this reaction step but would require certain mutations to change their function. GeneBank accession numbers for the gctA and gctB genes are listed above.

### 3.1.2.b Acyl-ACP Thioesterase

Acyl-ACP thioesterase enzymes convert an acyl-ACP to its corresponding acid. Such a transformation is required in steps H, L, T and AP of FIG. 10. Exemplary enzymes include the FatA and FatB isoforms of Arabidopsis thaliana (Salas et al, Arch Biochem Biophys 403:25-34 (2002)). The activities of these two proteins vary with carbon chain length, with FatA preferring oleyl-ACP and FatB preferring palmitoyl-ACP. See 3.1.2.14. A number of thioesterases with different chain length specificities are listed in WO 2008/113041 and are included in the table below [see p 126 Table 2A of patent]. For example, it has been shown previously that expression of medium chain plant thioesterases like FatB from Umbellularia californica in E. coli results in accumulation of high levels of medium chain fatty acids, primarily laurate (C12:0). Similarly, expression of Cuphea palustris FatB1 thioesterase in E. coli led to accumulation of C8-10:0 acyl-ACPs (Dehesh et al, Plant Physiol 110:203-10 (1996)). Similarly, Carthamus tinctorius thioesterase, when expressed in E. coli leads to >50 fold elevation in C 18:1 chain termination and release as free fatty acid (Knutzon et al, Plant Physiol 100:1751-58 (1992)). Methods for altering the substrate specificity of acyl-ACP thioesterases are also known in the art (for example, EP1605048).

| Protein | GenBank ID | GI Number | Organism |
|---|---|---|---|
| fatA | AEE76980.1 | 332643459 | Arabidopsis thaliana |
| fatB | AEE28300.1 | 332190179 | Arabidopsis thaliana |
| fatB2 | AAC49269.1 | 1292906 | Cuphea hookeriana |
| fatB1 | AAC49179.1 | 1215718 | Cuphea palustris |
| M96568.1: | AAA33019.1 | 404026 | Carthamus tinctorius |
| 94 . . . 1251 | | | |
| fatB1 | Q41635.1 | 8469218 | Umbellularia californica |
| tesA | AAC73596.1 | 1786702 | Escherichia coli |

### 4.2.1.a Hydro-Lyase

Several reactions in FIG. 10 depict dehydration reactions, including steps C, AB, AC and AD. Oleate hydratase enzymes catalyze the reversible hydration of non-activated alkenes to their corresponding alcohols. These enzymes represent additional suitable candidates as suggested in WO2011076691. Oleate hydratases from Elizabethkingia meningoseptica and Streptococcus pyogenes have been characterized (WO 2008/119735). Examples include the following proteins.

| Protein | GenBank ID | GI Number | Organism |
|---|---|---|---|
| OhyA | ACT54545.1 | 254031735 | Elizabethkingia meningoseptica |
| HMPREF0841_1446 | ZP_07461147.1 | 306827879 | Streptococcus pyogenes ATCC 10782 |
| P700755_13397 | ZP_01252267.1 | 91215295 | Psychroflexus torquis ATCC 700755 |
| RPB_2430 | YP_486046.1 | 86749550 | Rhodopseudomonas palustris |

3-Hydroxyacyl-ACP dehydratase enzymes are suitable candidates for dehydrating 3-hydroxybutyryl-ACP to crotonyl-ACP (step C of FIG. 10). Enzymes with this activity include FabA and FabZ of E. coli, which posses overlapping broad substrate specificities (Heath, J Biol Chem 271:1833-6 (1996)). Fatty acid synthase complexes, described above, also catalyze this reaction. The FabZ protein from Plasmodium falciparum has been crystallized (Kostrew et al, Protein Sci 14:1570-80 (2005)). Additional candidates are the mitochondrial 3-hydroxyacyl-ACP dehydratase encoded by Htd2p in yeast and TbHTD2 in Homo sapiens and Trypanosoma brucei (Kastanoitis et al, Mol Micro 53:1407-21 (2004); Kaija et al, FEBS Lett 582:729-33 (2008)).

| Protein | GenBank ID | GI Number | Organism |
|---|---|---|---|
| fabA | AAC74040.1 | 1787187 | Escherichia coli |
| fabZ | AAC73291.1 | 1786377 | Escherichia coli |
| PfFabZ | AAK83685.1 | 15080870 | Plasmodium falciparum |
| Htd2p | NP_011934.1 | 6321858 | Saccharomyces cerevisiae |
| HTD2 | P86397.1 | 281312149 | Homo sapiens |

Several additional hydratase and dehydratase enzymes have been described in the literature and represent suitable candidates for these steps. For example, many dehydratase enzymes catalyze the alpha, beta-elimination of water which involves activation of the alpha-hydrogen by an electron-withdrawing carbonyl, carboxylate, or CoA-thiol ester group and removal of the hydroxyl group from the beta-position (Buckel et al, J Bacteriol, 117:1248-60 (1974); Martins et al, PNAS 101:15645-9 (2004)). Exemplary enzymes include 2-(hydroxymethyl)glutarate dehydratase (EC 4.2.1.-), fumarase (EC 4.2.1.2), 3-dehydroquinate dehydratase (EC 4.2.1.10), cyclohexanone hydratase (EC 4.2.1.-) and 2-keto-4-pentenoate dehydratase (EC 4.2.1.80), citramalate hydrolyase and dimethylmaleate hydratase.

2-(Hydroxymethyl)glutarate dehydratase is a [4Fe-45]-containing enzyme that dehydrates 2-(hydroxymethyl)glutarate to 2-methylene-glutarate, studied for its role in nicontinate catabolism in Eubacterium barkeri (formerly Clostridium barkeri) (Alhapel et al., Proc Natl Acad Sci 103:12341-6 (2006)) Similar enzymes with high sequence homology are found in Bacteroides capillosus, Anaerotruncus colihominis, and Natranaerobius thermophilius. These enzymes are homologous to the alpha and beta subunits of [4Fe-45]-containing bacterial serine dehydratases (e.g., E. coli enzymes encoded by tdcG, sdhB, and sdaA). An enzyme with similar functionality in E. barkeri is dimethylmaleate hydratase, a reversible Fe²⁺-dependent and oxygen-sensitive enzyme in the aconitase family that hydrates dimethylmaeate to form (2R,3S)-2,3-dimethylmalate. This enzyme is encoded by dmdAB (Alhapel et al., Proc Natl Acad Sci USA 103:12341-6 (2006); Kollmann-Koch et al., Hoppe Seylers. Z. Physiol Chem. 365:847-857 (1984)).

| Protein | GenBank ID | GI Number | Organism |
|---|---|---|---|
| hmd | ABC88407.1 | 86278275 | Eubacterium barkeri |
| BACCAP_02294 | ZP_02036683.1 | 154498305 | Bacteroides capillosus |
| ANACOL_02527 | ZP_02443222.1 | 167771169 | Anaerotruncus |
| | | | colihominis |
| NtherDRAFT_ | ZP_02852366.1 | 169192667 | Natranaerobius |
| 2368 | | | thermophilus |
| dmdA | ABC88408 | 86278276 | Eubacterium barkeri |
| dmdB | ABC88409 | 86278277 | Eubacterium barkeri |

Fumarate hydratase (EC 4.2.1.2) enzymes naturally catalyze the reversible hydration of fumarate to malate. Although the ability of fumarate hydratase to react with 3-oxobutanol as a substrate has not been described in the literature, a wealth of structural information is available for this enzyme and other researchers have successfully engineered the enzyme to alter activity, inhibition and localization (Weaver, 61:1395-1401 (2005)). E. coli has three fumarases: FumA, FumB, and FumC that are regulated by growth conditions FumB is oxygen sensitive and only active under anaerobic conditions. FumA is active under microanaerobic conditions, and FumC is the only active enzyme in aerobic growth (Tseng et al., J Bacteriol, 183:461-467 (2001); Woods et al., 954:14-26 (1988); Guest et al., J Gen Microbiol 131:2971-2984 (1985)). Additional enzyme candidates are found in Campylobacter jejuni (Smith et al., Int. J Biochem. Cell Biol 31:961-975 (1999)), Thermus thermophilus (Mizobata et al., Arch. Biochem. Biophys. 355:49-55 (1998)) and Rattus norvegicus (Kobayashi et al., J Biochem, 89:1923-1931 (1981)) Similar enzymes with high sequence homology include fum1 from Arabidopsis thaliana and fumC from Corynebacterium glutamicum. The MmcBC fumarase from Pelotomaculum thermopropionicum is another class of fumarase with two subunits (Shimoyama et al., FEMS Microbiol Lett, 270:207-213 (2007)).

| Protein | GenBank ID | GI Number | Organism |
|---|---|---|---|
| fumA | NP_416129.1 | 16129570 | Escherichia coli |
| fumB | NP_418546.1 | 16131948 | Escherichia coli |
| fumC | NP_416128.1 | 16129569 | Escherichia coli |
| fumC | O69294 | 9789756 | Campylobacter jejuni |
| fumC | P84127 | 75427690 | Thermus thermophilus |
| fumH | P14408 | 120605 | Rattus norvegicus |
| fum1 | P93033 | 39931311 | Arabidopsis thaliana |
| fumC | Q8NRN8 | 39931596 | Corynebacterium glutamicum |
| MmcB | YP_001211906 | 147677691 | Pelotomaculum thermopropionicum |
| MmcC | YP_001211907 | 147677692 | Pelotomaculum thermopropionicum |

Dehydration of 4-hydroxy-2-oxovalerate to 2-oxopentenoate is catalyzed by 4-hydroxy-2-oxovalerate hydratase (EC 4.2.1.80). This enzyme participates in aromatic degradation pathways and is typically co-transcribed with a gene encoding an enzyme with 4-hydroxy-2-oxovalerate aldolase activity. Exemplary gene products are encoded by mhpD of E. coli (Ferrandez et al., J Bacteriol. 179:2573-2581 (1997); Pollard et al., Eur J Biochem. 251:98-106 (1998)), todG and cmtF of Pseudomonas putida (Lau et al., Gene 146:7-13 (1994); Eaton, J Bacteriol. 178:1351-1362 (1996)), cnbE of Comamonas sp. CNB-1 (Ma et al., Appl Environ Microbiol 73:4477-4483 (2007)) and mhpD of Burkholderia xenovorans (Wang et al., FEBS J 272:966-974 (2005)). A closely related enzyme, 2-oxohepta-4-ene-1,7-dioate hydratase, participates in 4-hydroxyphenylacetic acid degradation, where it converts 2-oxo-hept-4-ene-1,7-dioate (OHED) to 2-oxo-4-hydroxy-hepta-1,7-dioate using magnesium as a cofactor (Burks et al., J. Am. Chem. Soc. 120: (1998)). OHED hydratase enzyme candidates have been identified and characterized in E. coli C (Roper et al., Gene 156:47-51 (1995); Izumi et al., J Mol. Biol. 370:899-911 (2007)) and E. coli W (Prieto et al., J Bacteriol. 178:111-120 (1996)). Sequence comparison reveals homologs in a wide range of bacteria, plants and animals. Enzymes with highly similar sequences are contained in Klebsiella pneumonia (91% identity, eval=2e-138) and Salmonella enterica (91% identity, eval=4e-138), among others.

| | GenBank | | |
|---|---|---|---|
| Protein | Accession No. | GI No. | Organism |
| mhpD | AAC73453.2 | 87081722 | Escherichia coli |
| cmtF | AAB62293.1 | 1263188 | Pseudomonas putida |
| todG | AAA61942.1 | 485738 | Pseudomonas putida |
| cnbE | YP_001967714.1 | 190572008 | Comamonas sp. CNB-1 |
| mhpD | Q13VU0 | 123358582 | Burkholderia xenovorans |
| hpcG | CAA57202.1 | 556840 | Escherichia coli C |
| hpaH | CAA86044.1 | 757830 | Escherichia coli W |
| hpaH | ABR80130.1 | 150958100 | Klebsiella pneumoniae |
| Sari_01896 | ABX21779.1 | 160865156 | Salmonella enterica |

Another enzyme candidate is citramalate hydrolyase (EC 4.2.1.34), an enzyme that naturally dehydrates 2-methylmalate to mesaconate. This enzyme has been studied in Methanocaldococcus jannaschii in the context of the pyruvate pathway to 2-oxobutanoate, where it has been shown to have a broad substrate range (Drevland et al., J Bacteriol. 189:4391-4400 (2007)). This enzyme activity was also detected in Clostridium tetanomorphum, Morganella morganii, Citrobacter amalonaticus where it is thought to participate in glutamate degradation (Kato et al., Arch. Microbiol 168:457-463 (1997)). The M. jannaschii protein sequence does not bear significant homology to genes in these organisms.

| Protein | GenBank ID | GI Number | Organism |
|---|---|---|---|
| leuD | Q58673.1 | 3122345 | Methanocaldococcus jannaschii |

Dimethylmaleate hydratase (EC 4.2.1.85) is a reversible Fe^{e}′-dependent and oxygen-sensitive enzyme in the aconitase family that hydrates dimethylmaeate to form (2R,3S)-2,3-dimethylmalate. This enzyme is encoded by dmdAB in Eubacterium barkeri (Alhapel et al., supra; Kollmann-Koch et al., Hoppe Seylers. Z. Physiol Chem. 365:847-857 (1984)).

| Protein | GenBank ID | GI Number | Organism |
|---|---|---|---|
| dmdA | ABC88408 | 86278276 | Eubacterium barkeri |
| dmdB | ABC88409.1 | 86278277 | Eubacterium barkeri |

Oleate hydratases represent additional suitable candidates as suggested in WO2011076691. Examples include the following proteins.

| Protein | GenBank ID | GI Number | Organism |
|---|---|---|---|
| OhyA | ACT54545.1 | 254031735 | Elizabethkingia meningoseptica |
| HMPREF0841_1446 | ZP_07461147.1 | 306827879 | Streptococcus pyogenes ATCC 10782 |
| P700755_13397 | ZP_01252267.1 | 91215295 | Psychroflexus torquis ATCC 700755 |
| RPB_2430 | YP_486046.1 | 86749550 | Rhodopseudomonas palustris |

Enoyl-CoA hydratases (EC 4.2.1.17) catalyze the dehydration of a range of 3-hydroxyacyl-CoA substrates (Roberts et al., Arch. Microbiol 117:99-108 (1978); Agnihotri et al., Bioorg. Med. Chem. 11:9-20 (2003); Conrad et al., J Bacteriol. 118:103-111 (1974)). The enoyl-CoA hydratase of Pseudomonas putida, encoded by ech, catalyzes the conversion of 3-hydroxybutyryl-CoA to crotonyl-CoA (Roberts et al., Arch. Microbiol 117:99-108 (1978)). This transformation is also catalyzed by the crt gene product of Clostridium acetobutylicum, the crt1 gene product of C. kluyveri, and other clostridial organisms Atsumi et al., Metab Eng 10:305-311 (2008); Boynton et al., J Bacteriol. 178:3015-3024 (1996); Hillmer et al., FEBS Lett. 21:351-354 (1972)). Additional enoyl-CoA hydratase candidates are phaA and phaB, of P. putida, and paaA and paaB from P. fluorescens (Olivera et al., Proc. Natl. Acad. Sci U.S.A 95:6419-6424 (1998)). The gene product of pimF in Rhodopseudomonas palustris is predicted to encode an enoyl-CoA hydratase that participates in pimeloyl-CoA degradation (Harrison et al., Microbiology 151:727-736 (2005)). Lastly, a number of Escherichia coli genes have been shown to demonstrate enoyl-CoA hydratase functionality including maoC (Park et al., J Bacteriol. 185:5391-5397 (2003)), paaF (Ismail et al., Eur. J Biochem. 270:3047-3054 (2003); Park et al., Appl. Biochem. Biotechnol 113-116:335-346 (2004); Park et al., Biotechnol Bioeng 86:681-686 (2004)) and paaG (Ismail et al., Eur. J Biochem. 270:3047-3054 (2003); Park and Lee, Appl. Biochem. Biotechnol 113-116:335-346 (2004); Park and Yup, Biotechnol Bioeng 86:681-686 (2004)).

| Protein | GenBank No. | GI No. | Organism |
|---|---|---|---|
| ech | NP_745498.1 | 26990073 | Pseudomonas putida |
| crt | NP_349318.1 | 15895969 | Clostridium acetobutylicum |
| crt1 | YP_001393856 | 153953091 | Clostridium kluyveri |
| phaA | ABF82233.1 | 26990002 | Pseudomonas putida |
| phaB | ABF82234.1 | 26990001 | Pseudomonas putida |
| paaA | NP_745427.1 | 106636093 | Pseudomonas fluorescens |
| paaB | NP_745426.1 | 106636094 | Pseudomonas fluorescens |
| maoC | NP_415905.1 | 16129348 | Escherichia coli |
| paaF | NP_415911.1 | 16129354 | Escherichia coli |
| paaG | NP_415912.1 | 16129355 | Escherichia coli |

Alternatively, the E. coli gene products of fadA and fadB encode a multienzyme complex involved in fatty acid oxidation that exhibits enoyl-CoA hydratase activity (Yang et al., Biochemistry 30:6788-6795 (1991); Yang, J Bacteriol. 173:7405-7406 (1991); Nakahigashi et al., Nucleic Acids Res. 18:4937 (1990)). Knocking out a negative regulator encoded by fadR can be utilized to activate the fadB gene product (Sato et al., J Biosci. Bioeng 103:38-44 (2007)). The fadI and fadJ genes encode similar functions and are naturally expressed under anaerobic conditions (Campbell et al., Mol. Microbiol 47:793-805 (2003)).

| Protein | GenBank ID | GI Number | Organism |
|---|---|---|---|
| fadA | YP_026272.1 | 49176430 | Escherichia coli |
| fadB | NP_418288.1 | 16131692 | Escherichia coli |
| fadI | NP_416844.1 | 16130275 | Escherichia coli |
| fadJ | NP_416843.1 | 16130274 | Escherichia coli |
| fadR | NP_415705.1 | 16129150 | Escherichia coli |

### 6.2.1.a CoA Synthase (Acid-Thiol Ligase)

The conversion of acyl-CoA substrates to their acid products can be catalyzed by a CoA acid-thiol ligase or CoA synthetase in the 6.2.1 family of enzymes, several of which are reversible. These reactions include Steps E, Y, and AF of FIG. 10. Several enzymes catalyzing CoA acid-thiol ligase or CoA synthetase activities have been described in the literature and represent suitable candidates for these steps.

For example, ADP-forming acetyl-CoA synthetase (ACD, EC 6.2.1.13) is an enzyme that couples the conversion of acyl-CoA esters to their corresponding acids with the concomitant synthesis of ATP. ACD I from Archaeoglobus fulgidus, encoded by AF1211, was shown to operate on a variety of linear and branched-chain substrates including isobutyrate, isopentanoate, and fumarate (Musfeldt et al., J Bacteriol. 184:636-644 (2002)). A second reversible ACD in Archaeoglobus fulgidus, encoded by AF 1983, was also shown to have a broad substrate range with high activity on cyclic compounds phenylacetate and indoleacetate (Musfeldt and Schonheit, J Bacteriol. 184:636-644 (2002)). The enzyme from Haloarcula marismortui (annotated as a succinyl-CoA synthetase) accepts propionate, butyrate, and branched-chain acids (isovalerate and isobutyrate) as substrates, and was shown to operate in the forward and reverse directions (Brasen et al., Arch Microbiol 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen et al, supra). Directed evolution or engineering can be used to modify this enzyme to operate at the physiological temperature of the host organism. The enzymes from A. fulgidus, H. marismortui and P. aerophilum have all been cloned, functionally expressed, and characterized in E. coli (Brasen and Schonheit, supra; Musfeldt and Schonheit, J Bacteriol. 184:636-644 (2002)). An additional candidate is succinyl-CoA synthetase, encoded by sucCD of E. coli and LSC1 and LSC2 genes of Saccharomyces cerevisiae. These enzymes catalyze the formation of succinyl-CoA from succinate with the concomitant consumption of one ATP in a reaction which is reversible in vivo (Buck et al., Biochemistry 24:6245-6252 (1985)). The acyl CoA ligase from Pseudomonas putida has been demonstrated to work on several aliphatic substrates including acetic, propionic, butyric, valeric, hexanoic, heptanoic, and octanoic acids and on aromatic compounds such as phenylacetic and phenoxyacetic acids (Fernandez-Valverde et al., Appl. Environ. Microbiol. 59:1149-1154 (1993)). A related enzyme, malonyl CoA synthetase (6.3.4.9) from Rhizobium leguminosarum could convert several diacids, namely, ethyl-, propyl-, allyl-, isopropyl-, dimethyl-, cyclopropyl-, cyclopropylmethylene-, cyclobutyl-, and benzyl-malonate into their corresponding monothioesters (Pohl et al., J. Am. Chem. Soc. 123:5822-5823 (2001)).

| Protein | GenBank ID | GI Number | Organism |
|---|---|---|---|
| AF1211 | NP_070039.1 | 11498810 | Archaeoglobus fulgidus |
| AF1983 | NP_070807.1 | 11499565 | Archaeoglobus fulgidus |
| Scs | YP_135572.1 | 55377722 | Haloarcula marismortui |
| PAE3250 | NP_560604.1 | 18313937 | Pyrobaculum aerophilum str. IM2 |
| sucC | NP_415256.1 | 16128703 | Escherichia coli |
| sucD | AAC73823.1 | 1786949 | Escherichia coli |
| LSC1 | NP_014785 | 6324716 | Saccharomyces cerevisiae |
| LSC2 | NP_011760 | 6321683 | Saccharomyces cerevisiae |
| paaF | AAC24333.2 | 22711873 | Pseudomonas putida |
| matB | AAC83455.1 | 3982573 | Rhizobium leguminosarum |

Another candidate enzyme for these steps is 6-carboxyhexanoate-CoA ligase, also known as pimeloyl-CoA ligase (EC 6.2.1.14), which naturally activates pimelate to pimeloyl-CoA during biotin biosynthesis in gram-positive bacteria. The enzyme from Pseudomonas mendocina, cloned into E. coli, was shown to accept the alternate substrates hexanedioate and nonanedioate (Binieda et al., Biochem. J 340 (Pt 3):793-801 (1999)). Other candidates are found in Bacillus subtilis (Bower et al., J Bacteriol. 178:4122-4130 (1996)) and Lysinibacillus sphaericus (formerly Bacillus sphaericus) (Ploux et al., Biochem. J 287 (Pt 3):685-690 (1992)).

| Protein | GenBank ID | GI Number | Organism |
|---|---|---|---|
| bioW | NP_390902.2 | 50812281 | Bacillus subtilis |
| bioW | CAA10043.1 | 3850837 | Pseudomonas mendocina |
| bioW | P22822.1 | 115012 | Bacillus sphaericus |

Additional CoA-ligases include the rat dicarboxylate-CoA ligase for which the sequence is yet uncharacterized (Vamecq et al., Biochem. J 230:683-693 (1985)), either of the two characterized phenylacetate-CoA ligases from P. chrysogenum (Lamas-Maceiras et al., Biochem. J 395:147-155 (2006); Wang et al., 360:453-458 (2007)), the phenylacetate-CoA ligase from Pseudomonas putida (Martinez-Blanco et al., J Biol Chem 265:7084-7090 (1990)) and the 6-carboxyhexanoate-CoA ligase from Bacillus subtilis (Bower et al. J Bacteriol 178(14):4122-4130 (1996)). Acetoacetyl-CoA synthetases from Mus musculus (Hasegawa et al., Biochim Biophys Acta 1779:414-419 (2008)) and Homo sapiens (Ohgami et al., Biochem. Pharmacol. 65:989-994 (2003)) naturally catalyze the ATP-dependent conversion of acetoacetate into acetoacetyl-CoA.

| Protein | Accession No. | GI No. | Organism |
|---|---|---|---|
| phl | CAJ15517.1 | 77019264 | Penicillium chrysogenum |
| phlB | ABS19624.1 | 152002983 | Penicillium chrysogenum |
| paaF | AAC24333.2 | 22711873 | Pseudomonas putida |
| bioW | NP_390902.2 | 50812281 | Bacillus subtilis |
| AACS | NP_084486.1 | 21313520 | Mus musculus |
| AACS | NP_076417.2 | 31982927 | Homo sapiens |

Like enzymes in other classes, certain enzymes in the EC class 6.2.1 have been determined to have broad substrate specificity. The acyl CoA ligase from Pseudomonas putida has been demonstrated to work on several aliphatic substrates including acetic, propionic, butyric, valeric, hexanoic, heptanoic, and octanoic acids and on aromatic compounds such as phenylacetic and phenoxyacetic acids (Fernandez-Valverde et al., Applied and Environmental Microbiology 59:1149-1154 (1993)). A related enzyme, malonyl CoA synthetase (6.3.4.9) from Rhizobium trifolii could convert several diacids, namely, ethyl-, propyl-, allyl-, isopropyl-, dimethyl-, cyclopropyl-, cyclopropylmethylene-, cyclobutyl-, and benzyl-malonate into their corresponding monothioesters (Pohl et al., J. Am. Chem. Soc. 123:5822-5823 (2001)).

### FIG. 1, Step T—Acetyl-CoA Carboxylase

Several pathways shown in FIG. 10, in particular, those utilizing an acetoacetyl-CoA synthase (Step AS of FIG. 10, Step U of FIGS. 1 and 2) can also be combined with an acetyl-CoA carboxylase to form malonyl-CoA. This reaction includes Step T of FIGS. 1 and 2. Exemplary acetyl-CoA carboxylase enzymes are described in further detail below.

Acetyl-CoA carboxylase (EC 6.4.1.2) catalyzes the ATP-dependent carboxylation of acetyl-CoA to malonyl-CoA. This enzyme is biotin dependent and is the first reaction of fatty acid biosynthesis initiation in several organisms. Exemplary enzymes are encoded by accABCD of E. coli (Davis et al, J Biol Chem 275:28593-8 (2000)), ACC1 of Saccharomyces cerevisiae and homologs (Sumper et al, Methods Enzym 71:34-7 (1981)).

| Protein | GenBank ID | GI Number | Organism |
|---|---|---|---|
| ACC1 | CAA96294.1 | 1302498 | Saccharomyces |
| | | | cerevisiae |
| KLLA0F06072g | XP_455355.1 | 50310667 | Kluyveromyces |
| | | | lactis |
| ACC1 | XP_718624.1 | 68474502 | Candida albicans |
| YALI0C11407p | XP_501721.1 | 50548503 | Yarrowia lipolytica |
| ANI_1_1724104 | XP_001395476.1 | 145246454 | Aspergillus niger |
| accA | AAC73296.1 | 1786382 | Escherichia coli |
| accB | AAC76287.1 | 1789653 | Escherichia coli |
| accC | AAC76288.1 | 1789654 | Escherichia coli |
| accD | AAC75376.1 | 1788655 | Escherichia coli |

### FIG. 10, Step AS—Acetoacetyl-CoA Synthase

The conversion of malonyl-CoA and acetyl-CoA substrates to acetoacetyl-CoA can be catalyzed by a CoA synthetase in the 2.3.1 family of enzymes. These reactions include Steps E, Y, and AF of FIG. 10. Several enzymes catalyzing the CoA synthetase activities have been described in the literature and represent suitable candidates for these steps.

3-Oxoacyl-CoA products such as acetoacetyl-CoA, 3-oxopentanoyl-CoA, 3-oxo-5-hydroxypentanoyl-CoA can be synthesized from acyl-CoA and malonyl-CoA substrates by 3-oxoacyl-CoA synthases (Steps 10AS). As enzymes in this class catalyze an essentially irreversible reaction, they are particularly useful for metabolic engineering applications for overproducing metabolites, fuels or chemicals derived from 3-oxoacyl-CoA intermediates such as acetoacetyl-CoA. Acetoacetyl-CoA synthase, for example, has been heterologously expressed in organisms that biosynthesize butanol (Lan et al, PNAS USA (2012)) and poly-(3-hydroxybutyrate) (Matsumoto et al, Biosci Biotech Biochem, 75:364-366 (2011). An acetoacetyl-CoA synthase (EC 2.3.1.194) enzyme (FhsA) has been characterized in the soil bacterium Streptomyces sp. CL190 where it participates in mevalonate biosynthesis (Okamura et al, PNAS USA 107:11265-70 (2010)). Other acetoacetyl-CoA synthase genes can be identified by sequence homology fhsA.

| Protein | GenBank ID | GI Number | Organism |
|---|---|---|---|
| fhsA | BAJ83474.1 | 325302227 | Streptomyces sp CL190 |
| AB183750.1: | BAD86806.1 | 57753876 | Streptomyces sp. KO-3988 |
| 11991 . . . | | | |
| 12971 | | | |
| epzT | ADQ43379.1 | 312190954 | Streptomyces cinnamonensis |
| ppzT | CAX48662.1 | 238623523 | Streptomyces anulatus |
| O3I_22085 | ZP_09840373.1 | 378817444 | Nocardia brasiliensis |

### FIG. 10, Step AT—Acetyl-CoA:Acetyl-CoA Acyltransferase (Acetoacetyl-CoA Thiolase)

Acetoacetyl-CoA thiolase (also known as acetyl-CoA acetyltransferase) converts two molecules of acetyl-CoA into one molecule each of acetoacetyl-CoA and CoA. Exemplary acetoacetyl-CoA thiolase enzymes include the gene products of atoB from E. coli (Martin et al., Nat. Biotechnol 21:796-802 (2003)), thlA and thlB from C. acetobutylicum (Hanai et al., Appl Environ Microbiol 73:7814-7818 (2007); Winzer et al., J. Mol. Microbiol Biotechnol 2:531-541 (2000), and ERG10 from S. cerevisiae Hiser et al., J. Biol. Chem. 269:31383-31389 (1994)). These genes/proteins are identified in the Table below.

| Gene | GenBank ID | GI Number | Organism |
|---|---|---|---|
| AtoB | NP_416728 | 16130161 | Escherichia coli |
| ThlA | NP_349476.1 | 15896127 | Clostridium acetobutylicum |
| ThlB | NP_149242.1 | 15004782 | Clostridium acetobutylicum |
| ERG10 | NP_015297 | 6325229 | Saccharomyces cerevisiae |

### FIG. 10, Step AU—4-Hydroxybutyryl-CoA Dehydratase

4-Hydroxybutyryl-CoA dehydratase catalyzes the reversible conversion of 4-hydroxybutyryl-CoA to crotonyl-CoA. This enzyme possesses an intrinsic vinylacetyl-CoA Δ-isomerase activity, shifting the double bond from the 3,4 position to the 2,3 position (Scherf et al., Eur. J BioChem. 215:421-429 (1993); and Scherf et al., Arch. Microbiol 161:239-245 (1994)). 4-Hydroxybutyrul-CoA dehydratase enzymes from C. aminobutyricum and C. kluyveri were purified, characterized, and sequenced at the N-terminus (Scherf et al., Eur. J BioChem. 215:421-429 (1993); and Scherf et al., Arch. Microbiol 161:239-245 (1994)). The C. kluyveri enzyme, encoded by abfD, was cloned, sequenced and expressed in E. coli (Gerhardt et al., Arch. Microbiol 174:189-199 (2000)). The abfD gene product from Porphyromonas gingivalis ATCC 33277 is closely related by sequence homology to the Clostridial gene products. These genes/proteins are identified in the Table below.

| Gene | GenBank ID | GI Number | Organism |
|---|---|---|---|
| abfD | YP_001396399.1 | 153955634 | Clostridium kluyveri DSM 555 |
| abfD | P55792 | 84028213 | Clostridium aminobutyricum |
| abfD | YP_001928843 | 188994591 | Porphyromonas gingivalis |
| | | | ATCC 33277 |

### Example V

### Enzymatic Pathways for Producing Butadiene from Crotyl Alcohol

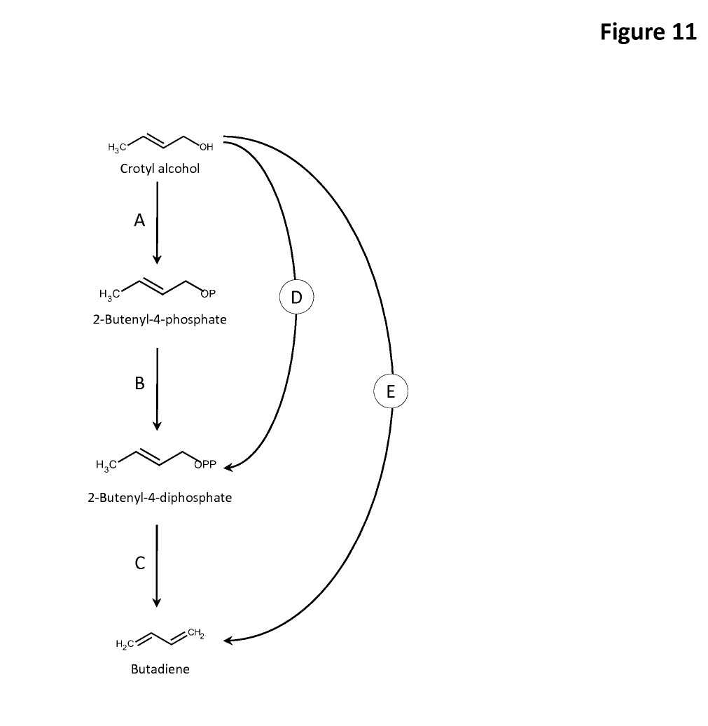

This example describes enzymatic pathways for converting crotyl alcohol to butadiene. The three pathways are shown in FIG. 11. In one pathway, crotyl alcohol is phosphorylated to 2-butenyl-4-phosphate by a crotyl alcohol kinase (Step A). The 2-butenyl-4-phosphate intermediate is again phosphorylated to 2-butenyl-4-diphosphate (Step B). A butadiene synthase enzyme catalyzes the conversion of 2-butenyl-4-diphosphate to butadiene (Step C). Such a butadiene synthase can be derived from a phosphate lyase enzyme such as isoprene synthase using methods, such as directed evolution, as described herein. In an alternate pathway, crotyl alcohol is directly converted to 2-butenyl-4-diphosphate by a diphosphokinase (step D). In yet another alternative pathway, crotyl alcohol can be converted to butadiene by a crotyl alcohol dehydratase (step E). Enzyme candidates for steps A-E are provided below.

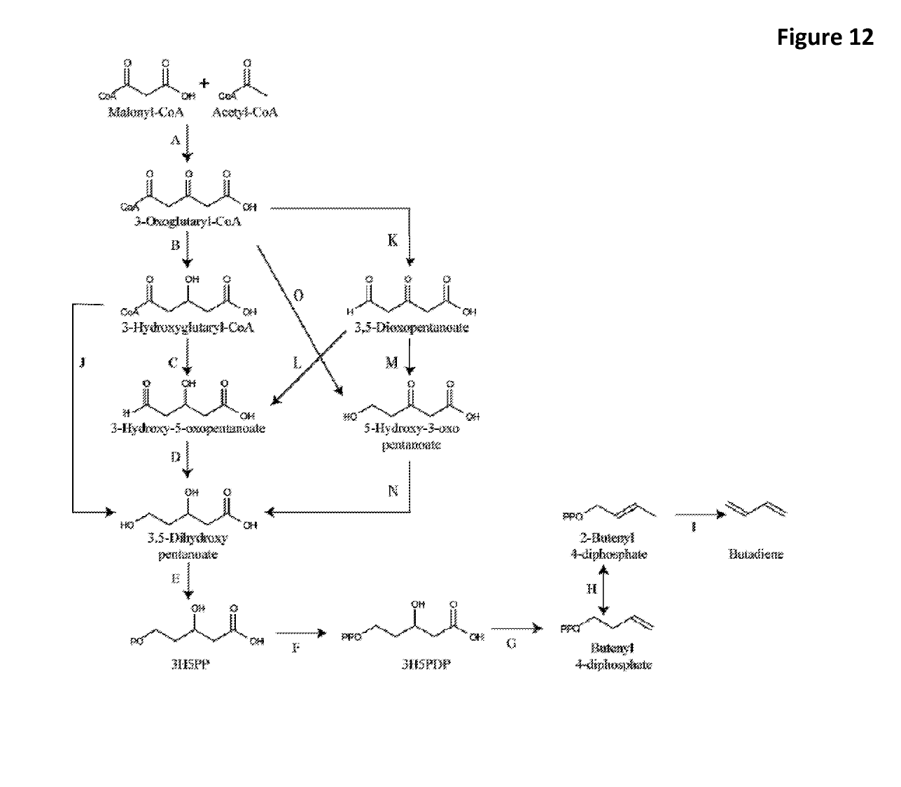

### Crotyl Alcohol Kinase (FIG. 12, Step A)

Crotyl alcohol kinase enzymes catalyze the transfer of a phosphate group to the hydroxyl group of crotyl alcohol. The enzymes described below naturally possess such activity or can be engineered to exhibit this activity. Kinases that catalyze transfer of a phosphate group to an alcohol group are members of the EC 2.7.1 enzyme class. The table below lists several useful kinase enzymes in the EC 2.7.1 enzyme class.

| Enzyme | |
|---|---|
| Commission | |
| Number | Enzyme Name |
| 2.7.1.1 | hexokinase |
| 2.7.1.2 | glucokinase |
| 2.7.1.3 | ketohexokinase |
| 2.7.1.4 | fructokinase |
| 2.7.1.5 | rhamnulokinase |
| 2.7.1.6 | galactokinase |
| 2.7.1.7 | mannokinase |
| 2.7.1.8 | glucosamine kinase |
| 2.7.1.10 | phosphoglucokinase |
| 2.7.1.11 | 6-phosphofructokinase |
| 2.7.1.12 | gluconokinase |
| 2.7.1.13 | dehydrogluconokinase |
| 2.7.1.14 | sedoheptulokinase |
| 2.7.1.15 | ribokinase |
| 2.7.1.16 | ribulokinase |
| 2.7.1.17 | xylulokinase |
| 2.7.1.18 | phosphoribokinase |
| 2.7.1.19 | phosphoribulokinase |
| 2.7.1.20 | adenosine kinase |
| 2.7.1.21 | thymidine kinase |
| 2.7.1.22 | ribosylnicotinamide kinase |
| 2.7.1.23 | NAD+ kinase |
| 2.7.1.24 | dephospho-CoA kinase |
| 2.7.1.25 | adenylyl-sulfate kinase |
| 2.7.1.26 | riboflavin kinase |
| 2.7.1.27 | erythritol kinase |
| 2.7.1.28 | triokinase |
| 2.7.1.29 | glycerone kinase |
| 2.7.1.30 | glycerol kinase |
| 2.7.1.31 | glycerate kinase |
| 2.7.1.32 | choline kinase |
| 2.7.1.33 | pantothenate kinase |
| 2.7.1.34 | pantetheine kinase |
| 2.7.1.35 | pyridoxal kinase |
| 2.7.1.36 | mevalonate kinase |
| 2.7.1.39 | homoserine kinase |
| 2.7.1.40 | pyruvate kinase |
| 2.7.1.41 | glucose-1-phosphate |
| | phosphodismutase |
| 2.7.1.42 | riboflavin |
| | phosphotransferase |
| 2.7.1.43 | glucuronokinase |
| 2.7.1.44 | galacturonokinase |
| 2.7.1.45 | 2-dehydro-3- |
| | deoxygluconokinase |
| 2.7.1.46 | L-arabinokinase |
| 2.7.1.47 | D-ribulokinase |
| 2.7.1.48 | uridine kinase |
| 2.7.1.49 | hydroxymethylpyrimidine kinase |
| 2.7.1.50 | hydroxyethylthiazole kinase |
| 2.7.1.51 | L-fuculokinase |
| 2.7.1.52 | fucokinase |
| 2.7.1.53 | L-xylulokinase |
| 2.7.1.54 | D-arabinokinase |
| 2.7.1.55 | allose kinase |
| 2.7.1.56 | 1-phosphofructokinase |
| 2.7.1.58 | 2-dehydro-3-deoxygalactonokinase |
| 2.7.1.59 | N-acetylglucosamine kinase |
| 2.7.1.60 | N-acylmannosamine kinase |
| 2.7.1.61 | acyl-phosphate-hexose |
| | phosphotransferase |
| 2.7.1.62 | phosphoramidate-hexose |
| | phosphotransferase |
| 2.7.1.63 | polyphosphate-glucose |
| | phosphotransferase |
| 2.7.1.64 | inositol 3-kinase |
| 2.7.1.65 | scyllo-inosamine 4-kinase |
| 2.7.1.66 | undecaprenol kinase |
| 2.7.1.67 | 1-phosphatidylinositol 4-kinase |
| 2.7.1.68 | 1-phosphatidylinositol-4-phosphate |
| | 5-kinase |
| 2.7.1.69 | protein-Np-phosphohistidine- |
| | sugar phosphotransferase |
| 2.7.1.70 | identical to EC 2.7.1.37. |
| 2.7.1.71 | shikimate kinase |
| 2.7.1.72 | streptomycin 6-kinase |
| 2.7.1.73 | inosine kinase |
| 2.7.1.74 | deoxycytidine kinase |
| 2.7.1.76 | deoxyadenosine kinase |
| 2.7.1.77 | nucleoside phosphotransferase |
| 2.7.1.78 | polynucleotide 5′-hydroxyl-kinase |
| 2.7.1.79 | diphosphate-glycerol |
| | phosphotransferase |
| 2.7.1.80 | diphosphate-serine |
| | phosphotransferase |
| 2.7.1.81 | hydroxylysine kinase |
| 2.7.1.82 | ethanolamine kinase |
| 2.7.1.83 | pseudouridine kinase |
| 2.7.1.84 | alkylglycerone kinase |
| 2.7.1.85 | β-glucoside kinase |
| 2.7.1.86 | NADH kinase |
| 2.7.1.87 | streptomycin 3″-kinase |
| 2.7.1.88 | dihydrostreptomycin-6-phosphate |
| | 3′a-kinase |
| 2.7.1.89 | thiamine kinase |
| 2.7.1.90 | diphosphate-fructose-6-phosphate |
| | 1-phosphotransferase |
| 2.7.1.91 | sphinganine kinase |
| 2.7.1.92 | 5-dehydro-2-deoxygluconokinase |
| 2.7.1.93 | alkylglycerol kinase |
| 2.7.1.94 | acylglycerol kinase |
| 2.7.1.95 | kanamycin kinase |
| 2.7.1.100 | S-methyl-5-thioribose kinase |
| 2.7.1.101 | tagatose kinase |
| 2.7.1.102 | hamamelose kinase |
| 2.7.1.103 | viomycin kinase |
| 2.7.1.105 | 6-phosphofructo-2-kinase |
| 2.7.1.106 | glucose-1,6-bisphosphate |
| | synthase |
| 2.7.1.107 | diacylglycerol kinase |
| 2.7.1.108 | dolichol kinase |
| 2.7.1.113 | deoxyguanosine kinase |
| 2.7.1.114 | AMP-thymidine kinase |
| 2.7.1.118 | ADP-thymidine kinase |
| 2.7.1.119 | hygromycin-B 7″-O-kinase |
| 2.7.1.121 | phosphoenolpyruvate-glycerone |
| | phosphotransferase |
| 2.7.1.122 | xylitol kinase |
| 2.7.1.127 | inositol-trisphosphate 3-kinase |
| 2.7.1.130 | tetraacyldisaccharide 4′-kinase |
| 2.7.1.134 | inositol-tetrakisphosphate 1- |
| | kinase |
| 2.7.1.136 | macrolide 2′-kinase |
| 2.7.1.137 | phosphatidylinositol 3-kinase |
| 2.7.1.138 | ceramide kinase |
| 2.7.1.140 | inositol-tetrakisphosphate 5- |
| | kinase |
| 2.7.1.142 | glycerol-3-phosphate-glucose |
| | phosphotransferase |
| 2.7.1.143 | diphosphate-purine nucleoside |
| | kinase |
| 2.7.1.144 | tagatose-6-phosphate kinase |
| 2.7.1.145 | deoxynucleoside kinase |
| 2.7.1.146 | ADP-dependent |
| | phosphofructokinase |
| 2.7.1.147 | ADP-dependent glucokinase |
| 2.7.1.148 | 4-(cytidine 5′-diphospho)-2-C- |
| | methyl-D-erythritol kinase |
| 2.7.1.149 | 1-phosphatidylinositol-5- |
| | phosphate 4-kinase |
| 2.7.1.150 | 1-phosphatidylinositol-3- |
| | phosphate 5-kinase |
| 2.7.1.151 | inositol-polyphosphate |
| | multikinase |
| 2.7.1.153 | phosphatidylinositol-4,5- |
| | bisphosphate 3-kinase |
| 2.7.1.154 | phosphatidylinositol-4-phosphate |
| | 3-kinase |
| 2.7.1.156 | adenosylcobinamide kinase |
| 2.7.1.157 | N-acetylgalactosamine kinase |
| 2.7.1.158 | inositol-pentakisphosphate 2- |
| | kinase |
| 2.7.1.159 | inositol-1,3,4-trisphosphate 5/6- |
| | kinase |
| 2.7.1.160 | 2′-phosphotransferase |
| 2.7.1.161 | CTP-dependent riboflavin kinase |
| 2.7.1.162 | N-acetylhexosamine 1-kinase |
| 2.7.1.163 | hygromycin B 4-O-kinase |
| 2.7.1.164 | O-phosphoseryl-tRNASec kinase |

Mevalonate kinase (EC 2.7.1.36) phosphorylates the terminal hydroxyl group of mevalonate. Gene candidates for this step include erg12 from S. cerevisiae, mvk from Methanocaldococcus jannaschi, MVK from Homo sapeins, and mvk from Arabidopsis thaliana col. Additional mevalonate kinase candidates include the feedback-resistant mevalonate kinase from the archeon Methanosarcina mazei (Primak et al, AEM, in press (2011)) and the Mvk protein from Streptococcus pneumoniae (Andreassi et al, Protein Sci, 16:983-9 (2007)). Mvk proteins from S. cerevisiae, S. pneumoniae and M. mazei were heterologously expressed and characterized in E. coli (Primak et al, supra). The S. pneumoniae mevalonate kinase was active on several alternate substrates including cylopropylmevalonate, vinylmevalonate and ethynylmevalonate (Kudoh et al, Bioorg Med Chem 18:1124-34 (2010)), and a subsequent study determined that the ligand binding site is selective for compact, electron-rich C(3)-substituents (Lefurgy et al, J Biol Chem 285:20654-63 (2010)).

| Protein | GenBank ID | GI Number | Organism |
|---|---|---|---|
| erg12 | CAA39359.1 | 3684 | Sachharomyces cerevisiae |
| mvk | Q58487.1 | 2497517 | Methanocaldococcus jannaschii |
| mvk | AAH16140.1 | 16359371 | Homo sapiens |
| mvk | NP_851084.1 | 30690651 | Arabidopsis thaliana |
| mvk | NP_633786.1 | 21227864 | Methanosarcina mazei |
| mvk | NP_357932.1 | 15902382 | Streptococcus pneumoniae |

Glycerol kinase also phosphorylates the terminal hydroxyl group in glycerol to form glycerol-3-phosphate. This reaction occurs in several species, including Escherichia coli, Saccharomyces cerevisiae, and Thermotoga maritima. The E. coli glycerol kinase has been shown to accept alternate substrates such as dihydroxyacetone and glyceraldehyde (Hayashi et al., J Biol. Chem. 242:1030-1035 (1967)). T, maritime has two glycerol kinases (Nelson et al., Nature 399:323-329 (1999)). Glycerol kinases have been shown to have a wide range of substrate specificity. Crans and Whiteside studied glycerol kinases from four different organisms (Escherichia coli, S. cerevisiae, Bacillus stearothermophilus, and Candida mycoderma) (Crans et al., J. Am. Chem. Soc. 107:7008-7018 (2010); Nelson et al., supra, (1999)). They studied 66 different analogs of glycerol and concluded that the enzyme could accept a range of substituents in place of one terminal hydroxyl group and that the hydrogen atom at C2 could be replaced by a methyl group. Interestingly, the kinetic constants of the enzyme from all four organisms were very similar.

| Protein | GenBank ID | GI Number | Organism |
|---|---|---|---|
| glpK | AP_003883.1 | 89110103 | Escherichia coli K12 |
| glpK1 | NP_228760.1 | 15642775 | Thermotoga maritime MSB8 |
| glpK2 | NP_229230.1 | 15642775 | Thermotoga maritime MSB8 |
| Gut1 | NP_011831.1 | 82795252 | Saccharomyces cerevisiae |

Homoserine kinase is another possible candidate. This enzyme is also present in a number of organisms including E. coli, Streptomyces sp, and S. cerevisiae. Homoserine kinase from E. coli has been shown to have activity on numerous substrates, including, L-2-amino, 1,4-butanediol, aspartate semialdehyde, and 2-amino-5-hydroxyvalerate (Huo et al., Biochemistry 35:16180-16185 (1996); Huo et al., Arch. Biochem. Biophys. 330:373-379 (1996)). This enzyme can act on substrates where the carboxyl group at the alpha position has been replaced by an ester or by a hydroxymethyl group. The gene candidates are:

| Protein | GenBank ID | GI Number | Organism |
|---|---|---|---|
| thrB | BAB96580.2 | 85674277 | Escherichia coli K12 |
| SACT1DRAFT_ | ZP_06280784.1 | 282871792 | Streptomyces sp. |
| 4809 | | | ACT-1 |
| Thr1 | AAA35154.1 | 172978 | Saccharomyces |
| | | | serevisiae |

### 2-Butenyl-4-Phosphate Kinase (FIG. 12, Step B)

2-Butenyl-4-phosphate kinase enzymes catalyze the transfer of a phosphate group to the phosphate group of 2-butenyl-4-phosphate. The enzymes described below naturally possess such activity or can be engineered to exhibit this activity. Kinases that catalyze transfer of a phosphate group to another phosphate group are members of the EC 2.7.4 enzyme class. The table below lists several useful kinase enzymes in the EC 2.7.4 enzyme class.

| Enzyme Commission Number | Enzyme Name |
|---|---|
| 2.7.4.1 | polyphosphate kinase |
| 2.7.4.2 | phosphomevalonate kinase |
| 2.7.4.3 | adenylate kinase |
| 2.7.4.4 | nucleoside-phosphate kinase |
| 2.7.4.6 | nucleoside-diphosphate kinase |
| 2.7.4.7 | phosphomethylpyrimidine kinase |
| 2.7.4.8 | guanylate kinase |
| 2.7.4.9 | dTMP kinase |
| 2.7.4.10 | nucleoside-triphosphate-adenylate kinase |
| 2.7.4.11 | (deoxy)adenylate kinase |
| 2.7.4.12 | T2-induced deoxynucleotide kinase |
| 2.7.4.13 | (deoxy)nucleoside-phosphate kinase |
| 2.7.4.14 | cytidylate kinase |
| 2.7.4.15 | thiamine-diphosphate kinase |
| 2.7.4.16 | thiamine-phosphate kinase |
| 2.7.4.17 | 3-phosphoglyceroyl-phosphate-polyphosphate phosphotransferase |
| 2.7.4.18 | farnesyl-diphosphate kinase |
| 2.7.4.19 | 5-methyldeoxycytidine-5′-phosphate kinase |
| 2.7.4.20 | dolichyl-diphosphate-polyphosphate phosphotransferase |
| 2.7.4.21 | inositol-hexakisphosphate kinase |
| 2.7.4.22 | UMP kinase |
| 2.7.4.23 | ribose 1,5-bisphosphate phosphokinase |
| 2.7.4.24 | diphosphoinositol-pentakisphosphate kinase |
| 2.7.4.- | Farnesyl monophosphate kinase |
| 2.7.4.- | Geranyl-geranyl monophosphate kinase |
| 2.7.4.- | Phytyl-phosphate kinase |

Phosphomevalonate kinase enzymes are of particular interest. Phosphomevalonate kinase (EC 2.7.4.2) catalyzes the analogous transformation to 2-butenyl-4-phosphate kinase. This enzyme is encoded by erg8 in Saccharomyces cerevisiae (Tsay et al., Mol. Cell Biol. 11:620-631 (1991)) and mvaK2 in Streptococcus pneumoniae, Staphylococcus aureus and Enterococcus faecalis (Doun et al., Protein Sci. 14:1134-1139 (2005); Wilding et al., J Bacteriol. 182:4319-4327 (2000)). The Streptococcus pneumoniae and Enterococcus faecalis enzymes were cloned and characterized in E. coli (Pilloff et al., J Biol. Chem. 278:4510-4515 (2003); Doun et al., Protein Sci. 14:1134-1139 (2005)). The S. pneumoniae phosphomevalonate kinase was active on several alternate substrates including cylopropylmevalonate phosphate, vinylmevalonate phosphate and ethynylmevalonate phosphate (Kudoh et al, Bioorg Med Chem 18:1124-34 (2010)).

| Protein | GenBank ID | GI Number | Organism |
|---|---|---|---|
| Erg8 | AAA34596.1 | 171479 | Saccharomyces cerevisiae |
| mvaK2 | AAG02426.1 | 9937366 | Staphylococcus aureus |
| mvaK2 | AAG02457.1 | 9937409 | Streptococcus pneumoniae |
| mvaK2 | AAG02442.1 | 9937388 | Enterococcus faecalis |

Farnesyl monophosphate kinase enzymes catalyze the CTP dependent phosphorylation of farnesyl monophosphate to farnesyl diphosphate. Similarly, geranylgeranyl phosphate kinase catalyzes CTP dependent phosphorylation. Enzymes with these activities were identified in the microsomal fraction of cultured Nicotiana tabacum (Thai et al, PNAS 96:13080-5 (1999)). However, the associated genes have not been identified to date.

### Butadiene Synthase (FIG. 12, Step C)

Butadiene synthase catalyzes the conversion of 2-butenyl-4-diphosphate to 1,3-butadiene. The enzymes described below naturally possess such activity or can be engineered to exhibit this activity. Carbon-oxygen lyases that operate on phosphates are found in the EC 4.2.3 enzyme class. The table below lists several useful enzymes in EC class 4.2.3.

| Enzyme Commission Number | Enzyme Name |
|---|---|
| 4.2.3.15 | Myrcene synthase |
| 4.2.3.26 | Linalool synthase |
| 4.2.3.27 | Isoprene synthase |
| 4.2.3.36 | Terpentriene sythase |
| 4.2.3.46 | (E,E)-alpha-Farnesene synthase |
| 4.2.3.47 | Beta-Farnesene synthase |
| 4.2.3.49 | Nerolidol synthase |

Particularly useful enzymes include isoprene synthase, myrcene synthase and farnesene synthase. Enzyme candidates are described below.

Isoprene synthase naturally catalyzes the conversion of dimethylallyl diphosphate to isoprene, but can also catalyze the synthesis of 1,3-butadiene from 2-butenyl-4-diphosphate. Isoprene synthases can be found in several organisms including Populus alba (Sasaki et al., FEBS Letters, 2005, 579 (11), 2514-2518), Pueraria montana (Lindberg et al., Metabolic Eng, 12(1):70-79 (2010); Sharkey et al., Plant Physiol., 137(2):700-712 (2005)), and Populus tremula×Populus alba, also called Populus canescens (Miller et al., Planta, 2001, 213 (3), 483-487). The crystal structure of the Populus canescens isoprene synthase was determined (Koksal et al, J Mol Biol 402:363-373 (2010)). Additional isoprene synthase enzymes are described in (Chotani et al., WO/2010/031079, Systems Using Cell Culture for Production of Isoprene; Cervin et al., US Patent Application 20100003716, Isoprene Synthase Variants for Improved Microbial Production of Isoprene).

| Protein | GenBank ID | GI Number | Organism |
|---|---|---|---|
| ispS | BAD98243.1 | 63108310 | Populus alba |
| ispS | AAQ84170.1 | 35187004 | Pueraria montana |
| ispS | CAC35696.1 | 13539551 | Populus tremula x Populus alba |

Myrcene synthase enzymes catalyze the dephosphorylation of geranyl diphosphate to beta-myrcene (EC 4.2.3.15). Exemplary myrcene synthases are encoded by MST2 of Solanum lycopersicum (van Schie et al, Plant Mol Biol 64:D473-79 (2007)), TPS-Myr of Picea abies (Martin et al, Plant Physiol 135:1908-27 (2004)) g-myr of Abies grandis (Bohlmann et al, J Biol Chem 272:21784-92 (1997)) and TPS10 of Arabidopsis thaliana (Bohlmann et al, Arch Biochem Biophys 375:261-9 (2000)). These enzymes were heterologously expressed in E. coli.

| Protein | GenBank ID | GI Number | Organism |
|---|---|---|---|
| MST2 | ACN58229.1 | 224579303 | Solanum lycopersicum |
| TPS-Myr | AAS47690.2 | 77546864 | Picea abies |
| G-myr | O24474.1 | 17367921 | Abies grandis |
| TPS10 | EC07543.1 | 330252449 | Arabidopsis thaliana |

Farnesyl diphosphate is converted to alpha-farnesene and beta-farnesene by alpha-farnesene synthase and beta-farnesene synthase, respectively. Exemplary alpha-farnesene synthase enzymes include TPS03 and TPS02 of Arabidopsis thaliana (Faldt et al, Planta 216:745-51 (2003); Huang et al, Plant Physiol 153:1293-310 (2010)), afs of Cucumis sativus (Mercke et al, Plant Physiol 135:2012-14 (2004), eafar of Malus×domestica (Green et al, Phytochem 68:176-88 (2007)) and TPS-Far of Picea abies (Martin, supra). An exemplary beta-farnesene synthase enzyme is encoded by TPS1 of Zea mays(Schnee et al, Plant Physiol 130:2049-60 (2002)).

| Protein | GenBank ID | GI Number | Organism |
|---|---|---|---|
| TPS03 | A4FVP2.1 | 205829248 | Arabidopsis thaliana |
| TPS02 | P0CJ43.1 | 317411866 | Arabidopsis thaliana |
| TPS-Far | AAS47697.1 | 44804601 | Picea abies |
| afs | AAU05951.1 | 51537953 | Cucumis sativus |
| eafar | Q84LB2.2 | 75241161 | Malus x domestica |
| TPS1 | Q84ZW8.1 | 75149279 | Zea mays |

### Crotyl Alcohol Diphosphokinase (FIG. 12, Step D)

Crotyl alcohol diphosphokinase enzymes catalyze the transfer of a diphosphate group to the hydroxyl group of crotyl alcohol. The enzymes described below naturally possess such activity or can be engineered to exhibit this activity. Kinases that catalyze transfer of a diphosphate group are members of the EC 2.7.6 enzyme class. The table below lists several useful kinase enzymes in the EC 2.7.6 enzyme class.

| Enzyme Commission Number | Enzyme Name |
|---|---|
| 2.7.6.1 | ribose-phosphate diphosphokinase |
| 2.7.6.2 | thiamine diphosphokinase |
| 2.7.6.3 | 2-amino-4-hydroxy-6-hydroxymethyldihydropteridine diphosphokinase |
| 2.7.6.4 | nucleotide diphosphokinase |
| 2.7.6.5 | GTP diphosphokinase |

Of particular interest are ribose-phosphate diphosphokinase enzymes which have been identified in Escherichia coli (Hove-Jenson et al., J Biol Chem, 1986, 261(15); 6765-71) and Mycoplasma pneumoniae M129 (McElwain et al, International Journal of Systematic Bacteriology, 1988, 38:417-423) as well as thiamine diphosphokinase enzymes. Exemplary thiamine diphosphokinase enzymes are found in Arabidopsis thaliana (Ajjawi, Plant Mol Biol, 2007, 65(1-2); 151-62).

| Protein | GenBank ID | GI Number | Organism |
|---|---|---|---|
| prs | NP 415725.1 | 16129170 | Escherichia coli |
| prsA | NP_109761.1 | 13507812 | Mycoplasma pneumoniae M129 |
| TPK1 | BAH19964.1 | 222424006 | Arabidopsis thaliana col |
| TPK2 | BAH57065.1 | 227204427 | Arabidopsis thaliana col |

### Crotyl Alcohol Dehydratase (FIG. 11, Step E)

Converting crotyl alcohol to butadiene using a crotyl alcohol dehydratase can include combining the activities of the enzymatic conversion of crotyl alcohol to 3-buten-2-ol then conversion of 3-buten-2-ol to butadiene. For example, a fusion protein or protein conjugate can be generated using well know methods in the art to generate a bi-functional (dual-functional) enzyme having both the isomerase and dehydratase activities. The fusion protein or protein conjugate can include at least the active domains of the enzymes (or respective genes) of the above two reactions. Alternatively, either or both steps can be done by chemical conversion, or by enzymatic conversion (in vivo or in vitro), or any combination. Enzymes having the desired activity for the conversion of 3-buten-2-ol to butadiene are provided elsewhere herein.

For the first step, the conversion of croytal alcohol to 3-buten-2-ol, enzymatic conversion can be catalyzed by a crotyl alcohol isomerase (classified as EC 5.4.4). A similar isomerization, the conversion of 2-methyl-3-buten-2-ol to 3-methyl-2-buten-1-ol, is catalyzed by cell extracts of Pseudomonas putida MB-1 (Malone et al, AEM 65 (6): 2622-30 (1999)). The extract may be used in vitro, or the protein or gene(s) associated with the isomerase activity can be isolated and used, even though they have not been identified to date.

### Example VI

### Pathways for the Production of Butadiene from Malonyl-CoA and Acetyl-CoA Via 3H5PP

This example describes enzymatic pathways for converting malonyl-CoA and acetyl-CoA to butadiene via 3H5PP. The five pathways are shown in FIG. 12. Enzyme candidates for steps A-O are provided below.

### Malonyl-CoA:Acetyl-CoA Acyltransferase (FIG. 12, Step A)

In Step A of the pathway described in FIG. 12, malonyl-CoA and acetyl-CoA are condensed to form 3-oxoglutaryl-CoA by malonyl-CoA:acetyl-CoA acyl transferase, a beta-keothiolase. Although no enzyme with activity on malonyl-CoA has been reported to date, a good candidate for this transformation is beta-ketoadipyl-CoA thiolase (EC 2.3.1.174), also called 3-oxoadipyl-CoA thiolase that converts beta-ketoadipyl-CoA to succinyl-CoA and acetyl-CoA, and is a key enzyme of the beta-ketoadipate pathway for aromatic compound degradation. The enzyme is widespread in soil bacteria and fungi including Pseudomonas putida (Harwood et al., J Bacteriol. 176:6479-6488 (1994)) and Acinetobacter calcoaceticus (Doten et al., J Bacteriol. 169:3168-3174 (1987)). The gene products encoded by pcaF in Pseudomonas strain B13 (Kaschabek et al., J Bacteriol. 184:207-215 (2002)), phaD in Pseudomonas putida U (Olivera et al., supra, (1998)), paaE in Pseudomonas fluorescens ST (Di Gennaro et al., Arch Microbiol. 88:117-125 (2007)), and paaJ from E. coli (Nogales et al., Microbiology, 153:357-365 (2007)) also catalyze this transformation. Several beta-ketothiolases exhibit significant and selective activities in the oxoadipyl-CoA forming direction including bkt from Pseudomonas putida, pcaF and bkt from Pseudomonas aeruginosa PAO1, bkt from Burkholderia ambifaria AMMD, paaJ from E. coli, and phaD from P. putida. These enzymes can also be employed for the synthesis of 3-oxoglutaryl-CoA, a compound structurally similar to 3-oxoadipyl-CoA.

| Protein | GenBank ID | GI Number | Organism |
|---|---|---|---|
| paaJ | NP_415915.1 | 16129358 | Escherichia coli |
| pcaF | AAL02407 | 17736947 | Pseudomonas knackmussii (B13) |
| phaD | AAC24332.1 | 3253200 | Pseudomonas putida |
| pcaF | AAA85138.1 | 506695 | Pseudomonas putida |
| pcaF | AAC37148.1 | 141777 | Acinetobacter calcoaceticus |
| paaE | ABF82237.1 | 106636097 | Pseudomonas fluorescens |
| bkt | YP_777652.1 | 115360515 | Burkholderia ambifaria AMMD |
| bkt | AAG06977.1 | 9949744 | Pseudomonas aeruginosa PAO1 |
| pcaF | AAG03617.1 | 9946065 | Pseudomonas aeruginosa PAO1 |

Another relevant beta-ketothiolase is oxopimeloyl-CoA:glutaryl-CoA acyltransferase (EC 2.3.1.16) that combines glutaryl-CoA and acetyl-CoA to form 3-oxopimeloyl-CoA. An enzyme catalyzing this transformation is found in Ralstonia eutropha (formerly known as Alcaligenes eutrophus), encoded by genes bktB and bktC (Slater et al., J. Bacteriol. 180:1979-1987 (1998); Haywood et al., FEMS Microbiology Letters 52:91-96 (1988)). The sequence of the BktB protein is known; however, the sequence of the BktC protein has not been reported. The pim operon of Rhodopseudomonas palustris also encodes a beta-ketothiolase, encoded by pimB, predicted to catalyze this transformation in the degradative direction during benzoyl-CoA degradation (Harrison et al., Microbiology 151:727-736 (2005)). A beta-ketothiolase enzyme candidate in S. aciditrophicus was identified by sequence homology to bktB (43% identity, evalue=1e-93).

| Protein | GenBank ID | GI Number | Organism |
|---|---|---|---|
| bktB | YP_725948 | 11386745 | Ralstonia eutropha |
| pimB | CAE29156 | 39650633 | Rhodopseudomonas palustris |
| syn_02642 | YP_462685.1 | 85860483 | Syntrophus aciditrophicus |

Beta-ketothiolase enzymes catalyzing the formation of beta-ketovaleryl-CoA from acetyl-CoA and propionyl-CoA can also be able to catalyze the formation of 3-oxoglutaryl-CoA. Zoogloea ramigera possesses two ketothiolases that can form β-ketovaleryl-CoA from propionyl-CoA and acetyl-CoA and R. eutropha has a β-oxidation ketothiolase that is also capable of catalyzing this transformation (Slater et al., J. Bacteriol, 180:1979-1987 (1998)). The sequences of these genes or their translated proteins have not been reported, but several candidates in R. eutropha, Z. ramigera, or other organisms can be identified based on sequence homology to bktB from R. eutropha. These include:

| Protein | GenBank ID | GI Number | Organism |
|---|---|---|---|
| phaA | YP_725941.1 | 113867452 | Ralstonia eutropha |
| h16_A1713 | YP_726205.1 | 113867716 | Ralstonia eutropha |
| pcaF | YP_728366.1 | 116694155 | Ralstonia eutropha |
| h16_B1369 | YP_840888.1 | 116695312 | Ralstonia eutropha |
| h16_A0170 | YP_724690.1 | 113866201 | Ralstonia eutropha |
| h16_A0462 | YP_724980.1 | 113866491 | Ralstonia eutropha |
| h16_A1528 | YP_726028.1 | 113867539 | Ralstonia eutropha |
| h16_B0381 | YP_728545.1 | 116694334 | Ralstonia eutropha |
| h16_B0662 | YP_728824.1 | 116694613 | Ralstonia eutropha |
| h16_B0759 | YP_728921.1 | 116694710 | Ralstonia eutropha |
| h16_B0668 | YP_728830.1 | 116694619 | Ralstonia eutropha |
| h16_A1720 | YP_726212.1 | 113867723 | Ralstonia eutropha |
| h16_A1887 | YP_726356.1 | 113867867 | Ralstonia eutropha |
| phbA | P07097.4 | 135759 | Zoogloea ramigera |
| bktB | YP_002005382.1 | 194289475 | Cupriavidus taiwanensis |
| Rmet_1362 | YP_583514.1 | 94310304 | Ralstonia metallidurans |
| Bphy_0975 | YP_001857210.1 | 186475740 | Burkholderia phymatum |

Additional candidates include beta-ketothiolases that are known to convert two molecules of acetyl-CoA into acetoacetyl-CoA (EC 2.1.3.9). Exemplary acetoacetyl-CoA thiolase enzymes include the gene products of atoB from E. coli (Martin et al., supra, (2003)), thlA and thlB from C. acetobutylicum (Hanai et al., supra, (2007); Winzer et al., supra, (2000)), and ERG10 from S. cerevisiae (Hiser et al., supra, (1994)).

| Protein | GenBank ID | GI Number | Organism |
|---|---|---|---|
| toB | NP_416728 | 16130161 | Escherichia coli |
| thlA | NP_349476.1 | 15896127 | Clostridium acetobutylicum |
| thlB | NP_149242.1 | 15004782 | Clostridium acetobutylicum |
| ERG10 | NP_015297 | 6325229 | Saccharomyces cerevisiae |

### 3-Oxoglutaryl-CoA Reductase (Ketone-Reducing) (FIG. 12, Step B)

This enzyme catalyzes the reduction of the 3-oxo group in 3-oxoglutaryl-CoA to the 3-hydroxy group in Step B of the pathway shown in FIG. 12.

3-Oxoacyl-CoA dehydrogenase enzymes convert 3-oxoacyl-CoA molecules into 3-hydroxyacyl-CoA molecules and are often involved in fatty acid beta-oxidation or phenylacetate catabolism. For example, subunits of two fatty acid oxidation complexes in E. coli, encoded by fadB and fadJ, function as 3-hydroxyacyl-CoA dehydrogenases (Binstock et al., Methods Enzymol. 71 Pt C:403-411 (1981)). Furthermore, the gene products encoded by phaC in Pseudomonas putida U (Olivera et al., supra, (1998)) and paaC in Pseudomonas fluorescens ST (Di et al., supra, (2007)) catalyze the reversible oxidation of 3-hydroxyadipyl-CoA to form 3-oxoadipyl-CoA, during the catabolism of phenylacetate or styrene. In addition, given the proximity in E. coli of paaH to other genes in the phenylacetate degradation operon (Nogales et al., supra, (2007)) and the fact that paaH mutants cannot grow on phenylacetate (Ismail et al., supra, (2003)), it is expected that the E. coli paaH gene encodes a 3-hydroxyacyl-CoA dehydrogenase.

| Protein | GenBank ID | GI Number | Organism |
|---|---|---|---|
| fadB | P21177.2 | 119811 | Escherichia coli |
| fadJ | P77399.1 | 3334437 | Escherichia coli |
| paaH | NP_415913.1 | 16129356 | Escherichia coli |
| phaC | NP_745425.1 | 26990000 | Pseudomonas putida |
| paaC | ABF82235.1 | 106636095 | Pseudomonas fluorescens |

3-Hydroxybutyryl-CoA dehydrogenase, also called acetoacetyl-CoA reductase, catalyzes the reversible NAD(P)H-dependent conversion of acetoacetyl-CoA to 3-hydroxybutyryl-CoA. This enzyme participates in the acetyl-CoA fermentation pathway to butyrate in several species of Clostridia and has been studied in detail (Jones and Woods, supra, (1986)). Enzyme candidates include hbd from C. acetobutylicum (Boynton et al., J Bacteriol. 178:3015-3024 (1996)), hbd from C. beijerinckii (Colby et al., Appl Environ. Microbiol 58:3297-3302 (1992)), and a number of similar enzymes from Metallosphaera sedula (Berg et al., supra, (2007)). The enzyme from Clostridium acetobutylicum, encoded by hbd, has been cloned and functionally expressed in E. coli (Youngleson et al., supra, (1989)). Yet other genes demonstrated to reduce acetoacetyl-CoA to 3-hydroxybutyryl-CoA are phbB from Zoogloea ramigera (Ploux et al., supra, (1988)) and phaB from Rhodobacter sphaeroides (Alber et al., supra, (2006)). The former gene is NADPH-dependent, its nucleotide sequence has been determined (Peoples and Sinskey, supra, (1989)) and the gene has been expressed in E. coli. Additional genes include hbd1 (C-terminal domain) and hbd2 (N-terminal domain) in Clostridium kluyveri (Hillmer and Gottschalk, Biochim. Biophys. Acta 3334:12-23 (1974)) and HSD17B10 in Bos taurus (WAKIL et al., supra, (1954)).

| Protein | GenBank ID | GI Number | Organism |
|---|---|---|---|
| hbd | NP_349314.1 | 15895965 | Clostridium acetobutylicum |
| hbd | AAM14586.1 | 20162442 | Clostridium beijerinckii |
| Msed_1423 | YP_001191505 | 146304189 | Metallosphaera sedula |
| Msed_0399 | YP_001190500 | 146303184 | Metallosphaera sedula |
| Msed_0389 | YP_001190490 | 146303174 | Metallosphaera sedula |
| Msed_1993 | YP_001192057 | 146304741 | Metallosphaera sedula |
| hbd2 | EDK34807.1 | 146348271 | Clostridium kluyveri |
| hbd1 | EDK32512.1 | 146345976 | Clostridium kluyveri |
| HSD17B10 | O02691.3 | 3183024 | Bos taurus |
| phaB | YP_353825.1 | 77464321 | Rhodobacter sphaeroides |
| phbB | P23238.1 | 130017 | Zoogloea ramigera |

### 3-Hydroxyglutaryl-CoA Reductase (Aldehyde Forming) (FIG. 12, Step C)

3-hydroxyglutaryl-CoA reductase reduces 3-hydroxyglutaryl-CoA to 3-hydroxy-5-oxopentanoate. Several acyl-CoA dehydrogenases reduce an acyl-CoA to its corresponding aldehyde (EC 1.2.1). Exemplary genes that encode such enzymes include the Acinetobacter calcoaceticus acr1 encoding a fatty acyl-CoA reductase (Reiser and Somerville, supra, (1997)), the Acinetobacter sp. M-1 fatty acyl-CoA reductase (Ishige et al., supra, (2002)), and a CoA- and NADP-dependent succinate semialdehyde dehydrogenase encoded by the sucD gene in Clostridium kluyveri (Sohling and Gottschalk, supra, (1996); Sohling and Gottschalk, supra, (1996)). SucD of P. gingivalis is another succinate semialdehyde dehydrogenase (Takahashi et al., supra, (2000)). The enzyme acylating acetaldehyde dehydrogenase in Pseudomonas sp, encoded by bphG, is yet another as it has been demonstrated to oxidize and acylate acetaldehyde, propionaldehyde, butyraldehyde, isobutyraldehyde and formaldehyde (Powlowski et al., supra, (1993)). In addition to reducing acetyl-CoA to ethanol, the enzyme encoded by adhE in Leuconostoc mesenteroides has been shown to oxidize the branched chain compound isobutyraldehyde to isobutyryl-CoA (Koo et al., Biotechnol Lett. 27:505-510 (2005)). Butyraldehyde dehydrogenase catalyzes a similar reaction, conversion of butyryl-CoA to butyraldehyde, in solventogenic organisms such as Clostridium saccharoperbutylacetonicum (Kosaka et al., Biosci. Biotechnol Biochem. 71:58-68 (2007)).

| Protein | GenBank ID | GI Number | Organism |
|---|---|---|---|
| acr1 | YP_047869.1 | 50086359 | Acinetobacter calcoaceticus |
| acr1 | AAC45217 | 1684886 | Acinetobacter baylyi |
| acr1 | BAB85476.1 | 18857901 | Acinetobacter sp. Strain M-1 |
| sucD | P38947.1 | 172046062 | Clostridium kluyveri |
| sucD | NP_904963.1 | 34540484 | Porphyromonas gingivalis |
| bphG | BAA03892.1 | 425213 | Pseudomonas sp |
| adhE | AAV66076.1 | 55818563 | Leuconostoc mesenteroides |
| bld | AAP42563.1 | 31075383 | Clostridium |
| | | | saccharoperbutylacetonicum |

An additional enzyme type that converts an acyl-CoA to its corresponding aldehyde is malonyl-CoA reductase which transforms malonyl-CoA to malonic semialdehyde. Malonyl-CoA reductase is a key enzyme in autotrophic carbon fixation via the 3-hydroxypropionate cycle in thermoacidophilic archael bacteria (Berg et al., supra, (2007b); Thauer, supra, (2007)). The enzyme utilizes NADPH as a cofactor and has been characterized in Metallosphaera and Sulfolobus spp (Alber et al., supra, (2006); Bugler et al., supra, (2002)). The enzyme is encoded by Msed_{—}0709 in Metallosphaera sedula (Alber et al., supra, (2006); Berg et al., supra, (2007b)). A gene encoding a malonyl-CoA reductase from Sulfolobus tokodaii was cloned and heterologously expressed in E. coli (Alber et al., supra, (2006)). This enzyme has also been shown to catalyze the conversion of methylmalonyl-CoA to its corresponding aldehyde (WO/2007/141208). Although the aldehyde dehydrogenase functionality of these enzymes is similar to the bifunctional dehydrogenase from Chloroflexus aurantiacus, there is little sequence similarity. Both malonyl-CoA reductase enzyme candidates have high sequence similarity to aspartate-semialdehyde dehydrogenase, an enzyme catalyzing the reduction and concurrent dephosphorylation of aspartyl-4-phosphate to aspartate semialdehyde. Additional gene candidates can be found by sequence homology to proteins in other organisms including Sulfolobus solfataricus and Sulfolobus acidocaldarius. Yet another acyl-CoA reductase (aldehyde forming) candidate is the ald gene from Clostridium beijerinckii (Toth et al., Appl Environ. Microbiol 65:4973-4980 (1999)). This enzyme has been reported to reduce acetyl-CoA and butyryl-CoA to their corresponding aldehydes. This gene is very similar to eutE that encodes acetaldehyde dehydrogenase of Salmonella typhimurium and E. coli (Toth et al., supra, (1999)).

| Protein | GenBank ID | GI Number | Organism |
|---|---|---|---|
| MSED_0709 | YP_001190808.1 | 146303492 | Metallosphaera sedula |
| mcr | NP_378167.1 | 15922498 | Sulfolobus tokodaii |
| asd-2 | NP_343563.1 | 15898958 | Sulfolobus solfataricus |
| Saci_2370 | YP_256941.1 | 70608071 | Sulfolobus acidocaldarius |
| Ald | AAT66436 | 9473535 | Clostridium beijerinckii |
| eutE | AAA80209 | 687645 | Salmonella typhimurium |
| eutE | P77445 | 2498347 | Escherichia coli |

### 3-Hydroxy-5-Oxopentanoate Reductase (FIG. 12, Step D)

This enzyme reduces the terminal aldehyde group in 3-hydroxy-5-oxopentanote to the alcohol group. Exemplary genes encoding enzymes that catalyze the conversion of an aldehyde to alcohol (i.e., alcohol dehydrogenase or equivalently aldehyde reductase, 1.1.1.a) include alrA encoding a medium-chain alcohol dehydrogenase for C2-C14 (Tani et al., supra, (2000)), ADH2 from Saccharomyces cerevisiae (Atsumi et al., supra, (2008)), yqhD from E. coli which has preference for molecules longer than C(3) (Sulzenbacher et al., supra, (2004)), and bdh I and bdh II from C. acetobutylicum which converts butyraldehyde into butanol (Walter et al., supra, (1992)). The gene product of yqhD catalyzes the reduction of acetaldehyde, malondialdehyde, propionaldehyde, butyraldehyde, and acrolein using NADPH as the cofactor (Perez et al., 283:7346-7353 (2008); Perez et al., J Biol. Chem. 283:7346-7353 (2008)). The adhA gene product from Zymomonas mobilis has been demonstrated to have activity on a number of aldehydes including formaldehyde, acetaldehyde, propionaldehyde, butyraldehyde, and acrolein (Kinoshita et al., Appl Microbiol Biotechnol 22:249-254 (1985)).

| Protein | GenBank ID | GI Number | Organism |
|---|---|---|---|
| alrA | BAB12273.1 | 9967138 | Acinetobacter sp. Strain M-1 |
| ADH2 | NP_014032.1 | 6323961 | Saccharomyces cerevisiae |
| yqhD | NP_417484.1 | 16130909 | Escherichia coli |
| bdh I | NP_349892.1 | 15896543 | Clostridium acetobutylicum |
| bdh II | NP_349891.1 | 15896542 | Clostridium acetobutylicum |
| adhA | YP_162971.1 | 56552132 | Zymomonas mobilis |

Enzymes exhibiting 4-hydroxybutyrate dehydrogenase activity (EC 1.1.1.61) also fall into this category. Such enzymes have been characterized in Ralstonia eutropha (Bravo et al., supra, (2004)), Clostridium kluyveri (Wolff and Kenealy, supra, (1995)) and Arabidopsis thaliana (Breitkreuz et al., supra, (2003)). The A. thaliana enzyme was cloned and characterized in yeast [12882961]. Yet another gene is the alcohol dehydrogenase adhI from Geobacillus thermoglucosidasius (Jeon et al., J Biotechnol 135:127-133 (2008)).

| Protein | GenBank ID | GI Number | Organism |
|---|---|---|---|
| 4hbd | YP_726053.1 | 113867564 | Ralstonia eutropha H16 |
| 4hbd | EDK35022.1 | 146348486 | Clostridium kluyveri |
| 4hbd | Q94B07 | 75249805 | Arabidopsis thaliana |
| adhI | AAR91477.1 | 40795502 | Geobacillus thermoglucosidasius |

Another exemplary enzyme is 3-hydroxyisobutyrate dehydrogenase (EC 1.1.1.31) which catalyzes the reversible oxidation of 3-hydroxyisobutyrate to methylmalonate semialdehyde. This enzyme participates in valine, leucine and isoleucine degradation and has been identified in bacteria, eukaryotes, and mammals. The enzyme encoded by P84067 from Thermus thermophilus HB8 has been structurally characterized (Lokanath et al., J Mol Biol 352:905-17 (2005)). The reversibility of the human 3-hydroxyisobutyrate dehydrogenase was demonstrated using isotopically-labeled substrate (Manning et al., Biochem J 231:481-4 (1985)). Additional genes encoding this enzyme include 3hidh in Homo sapiens (Hawes et al., Methods Enzymol 324:218-228 (2000)) and Oryctolagus cuniculus (Hawes et al., supra, (2000); Chowdhury et al., Biosci. Biotechnol Biochem. 60:2043-2047 (1996)), mmsb in Pseudomonas aeruginosa, and dhat in Pseudomonas putida (Aberhart et al., J Chem. Soc. [Perkin 1] 6:1404-1406 (1979); Chowdhury et al., supra, (1996); Chowdhury et al., Biosci. Biotechnol Biochem. 67:438-441 (2003)).

| Protein | GenBank ID | GI Number | Organism |
|---|---|---|---|
| P84067 | P84067 | 75345323 | Thermus thermophilus |
| mmsb | P28811.1 | 127211 | Pseudomonas aeruginosa |
| dhat | Q59477.1 | 2842618 | Pseudomonas putida |
| 3hidh | P31937.2 | 12643395 | Homo sapiens |
| 3hidh | P32185.1 | 416872 | Oryctolagus cuniculus |

The conversion of malonic semialdehyde to 3-HP can also be accomplished by two other enzymes: NADH-dependent 3-hydroxypropionate dehydrogenase and NADPH-dependent malonate semialdehyde reductase. An NADH-dependent 3-hydroxypropionate dehydrogenase is thought to participate in beta-alanine biosynthesis pathways from propionate in bacteria and plants (Rathinasabapathi B., Journal of Plant Pathology 159:671-674 (2002); Stadtman, J. Am. Chem. Soc. 77:5765-5766 (1955)). This enzyme has not been associated with a gene in any organism to date. NADPH-dependent malonate semialdehyde reductase catalyzes the reverse reaction in autotrophic CO2-fixing bacteria. Although the enzyme activity has been detected in Metallosphaera sedula, the identity of the gene is not known (Alber et al., supra, (2006)).

### 3,5-Dihydroxypentanoate Kinase (FIG. 12, Step E)

This enzyme phosphorylates 3,5-dihydroxypentanotae in FIG. 12 (Step E) to form 3-hydroxy-5-phosphonatooxypentanoate (3H5PP). This transformation can be catalyzed by enzymes in the EC class 2.7.1 that enable the ATP-dependent transfer of a phosphate group to an alcohol.

A good candidate for this step is mevalonate kinase (EC 2.7.1.36) that phosphorylates the terminal hydroxyl group of the methyl analog, mevalonate, of 3,5-dihydroxypentanote. Some gene candidates for this step are erg12 from S. cerevisiae, mvk from Methanocaldococcus jannaschi, MVK from Homo sapeins, and mvk from Arabidopsis thaliana col.

| Protein | GenBank ID | GI Number | Organism |
|---|---|---|---|
| erg12 | CAA39359.1 | 3684 | Sachharomyces cerevisiae |
| mvk | Q58487.1 | 2497517 | Methanocaldococcus jannaschii |
| mvk | AAH16140.1 | 16359371 | Homo sapiens |
| M\mvk | NP_851084.1 | 30690651 | Arabidopsis thaliana |

Glycerol kinase also phosphorylates the terminal hydroxyl group in glycerol to form glycerol-3-phosphate. This reaction occurs in several species, including Escherichia coli, Saccharomyces cerevisiae, and Thermotoga maritima. The E. coli glycerol kinase has been shown to accept alternate substrates such as dihydroxyacetone and glyceraldehyde (Hayashi and Lin, supra, (1967)). T, maritime has two glycerol kinases (Nelson et al., supra, (1999)). Glycerol kinases have been shown to have a wide range of substrate specificity. Crans and Whiteside studied glycerol kinases from four different organisms (Escherichia coli, S. cerevisiae, Bacillus stearothermophilus, and Candida mycoderma) (Crans and Whitesides, supra, (2010); Nelson et al., supra, (1999)). They studied 66 different analogs of glycerol and concluded that the enzyme could accept a range of substituents in place of one terminal hydroxyl group and that the hydrogen atom at C2 could be replaced by a methyl group. Interestingly, the kinetic constants of the enzyme from all four organisms were very similar. The gene candidates are:

| Protein | GenBank ID | GI Number | Organism |
|---|---|---|---|
| glpK | AP_003883.1 | 89110103 | Escherichia coli K12 |
| glpK1 | NP_228760.1 | 15642775 | Thermotoga maritime MSB8 |
| glpK2 | NP_229230.1 | 15642775 | Thermotoga maritime MSB8 |
| Gut1 | NP_011831.1 | 82795252 | Saccharomyces cerevisiae |

Homoserine kinase is another possible candidate that can lead to the phosphorylation of 3,5-dihydroxypentanoate. This enzyme is also present in a number of organisms including E. coli, Streptomyces sp, and S. cerevisiae. Homoserine kinase from E. coli has been shown to have activity on numerous substrates, including, L-2-amino, 1,4-butanediol, aspartate semialdehyde, and 2-amino-5-hydroxyvalerate (Huo and Viola, supra, (1996); Huo and Viola, supra, (1996)). This enzyme can act on substrates where the carboxyl group at the alpha position has been replaced by an ester or by a hydroxymethyl group. The gene candidates are:

| Protein | GenBank ID | GI Number | Organism |
|---|---|---|---|
| thrB | BAB96580.2 | 85674277 | Escherichia coli |
| | | | K12 |
| SACT1DRAFT_4809 | ZP_06280784.1 | 282871792 | Streptomyces sp. |
| | | | ACT-1 |
| Thr1 | AAA35154.1 | 172978 | Saccharomyces |
| | | | serevisiae |

### 3H5PP Kinase (FIG. 12, Step F)

Phosphorylation of 3H5PP to 3H5PDP is catalyzed by 3H5PP kinase (FIG. 12, Step F). Phosphomevalonate kinase (EC 2.7.4.2) catalyzes the analogous transformation in the mevalonate pathway. This enzyme is encoded by erg8 in Saccharomyces cerevisiae (Tsay et al., Mol. Cell Biol. 11:620-631 (1991)) and mvaK2 in Streptococcus pneumoniae, Staphylococcus aureus and Enterococcus faecalis (Doun et al., Protein Sci. 14:1134-1139 (2005); Wilding et al., J Bacteriol. 182:4319-4327 (2000)). The Streptococcus pneumoniae and Enterococcus faecalis enzymes were cloned and characterized in E. coli (Pilloff et al., J Biol. Chem. 278:4510-4515 (2003); Doun et al., Protein Sci. 14:1134-1139 (2005)).

| Protein | GenBank ID | GI Number | Organism |
|---|---|---|---|
| Erg8 | AAA34596.1 | 171479 | Saccharomyces cerevisiae |
| mvaK2 | AAG02426.1 | 9937366 | Staphylococcus aureus |
| mvaK2 | AAG02457.1 | 9937409 | Streptococcus pneumoniae |
| mvaK2 | AAG02442.1 | 9937388 | Enterococcus faecalis |

### 3H5PDP Decarboxylase (FIG. 12, Step G)

Butenyl 4-diphosphate is formed from the ATP-dependent decarboxylation of 3H5PDP by 3H5PDP decarboxylase (FIG. 12, Step G). Although an enzyme with this activity has not been characterized to date a similar reaction is catalyzed by mevalonate diphosphate decarboxylase (EC 4.1.1.33), an enzyme participating in the mevalonate pathway for isoprenoid biosynthesis. This reaction is catalyzed by MVD1 in Saccharomyces cerevisiae, MVD in Homo sapiens and MDD in Staphylococcus aureus and Trypsonoma brucei (Toth et al., J Biol. Chem. 271:7895-7898 (1996); Byres et al., J Mol. Biol. 371:540-553 (2007)).

| Protein | GenBank ID | GI Number | Organism |
|---|---|---|---|
| MVD1 | P32377.2 | 1706682 | Saccharomyces cerevisiae |
| MVD | NP_002452.1 | 4505289 | Homo sapiens |
| MDD | ABQ48418.1 | 147740120 | Staphylococcus aureus |
| MDD | EAN78728.1 | 70833224 | Trypsonoma brucei |

### Butenyl 4-Diphosphate Isomerase (FIG. 12, Step H)

Butenyl 4-diphosphate isomerase catalyzes the reversible interconversion of 2-butenyl-4-diphosphate and butenyl-4-diphosphate. The following enzymes can naturally possess this activity or can be engineered to exhibit this activity. Useful genes include those that encode enzymes that interconvert isopentenyl diphosphate and dimethylallyl diphosphate. These include isopentenyl diphosphate isomerase enzymes from Escherichia coli (Rodríguez-Concepción et al., FEBS Lett, 473(3):328-332), Saccharomyces cerevisiae (Anderson et al., J Biol Chem, 1989, 264(32); 19169-75), and Sulfolobus shibatae (Yamashita et al, Eur J Biochem, 2004, 271(6); 1087-93). The reaction mechanism of isomerization, catalyzed by the Idi protein of E. coli, has been characterized in mechanistic detail (de Ruyck et al., J Biol. Chem. 281:17864-17869 (2006)). Isopentenyl diphosphate isomerase enzymes from Saccharomyces cerevisiae, Bacillus subtilis and Haematococcus pluvialis have been heterologously expressed in E. coli (Laupitz et al., Eur. J Biochem. 271:2658-2669 (2004); Kajiwara et al., Biochem. J 324 (Pt 2):421-426 (1997)).

| Protein | GenBank ID | GI Number | Organism |
|---|---|---|---|
| Idi | NP_417365.1 | 16130791 | Escherichia coli |
| IDI1 | NP_015208.1 | 6325140 | Saccharomyces cerevisiae |
| Idi | BAC82424.1 | 34327946 | Sulfolobus shibatae |
| Idi | AAC32209.1 | 3421423 | Haematococcus pluvialis |
| Idi | BAB32625.1 | 12862826 | Bacillus subtilis |

### Butadiene Synthase (FIG. 12, Step I)

Butadiene synthase catalyzes the conversion of 2-butenyl-4-diphosphate to 1,3-butadiene. The enzymes described below naturally possess such activity or can be engineered to exhibit this activity. Isoprene synthase naturally catalyzes the conversion of dimethylallyl diphosphate to isoprene, but can also catalyze the synthesis of 1,3-butadiene from 2-butenyl-4-diphosphate. Isoprene synthases can be found in several organisms including Populus alba (Sasaki et al., FEBS Letters, 2005, 579 (11), 2514-2518), Pueraria montana (Lindberg et al., Metabolic Eng, 12(1):70-79 (2010); Sharkey et al., Plant Physiol., 137(2):700-712 (2005)), and Populus tremula×Populus alba (Miller et al., Planta, 213(3):483-487 (2001)). Additional isoprene synthase enzymes are described in (Chotani et al., WO/2010/031079, Systems Using Cell Culture for Production of Isoprene; Cervin et al., US Patent Application 20100003716, Isoprene Synthase Variants for Improved Microbial Production of Isoprene).

| Protein | GenBank ID | GI Number | Organism |
|---|---|---|---|
| ispS | BAD98243.1 | 63108310 | Populus alba |
| ispS | AAQ84170.1 | 35187004 | Pueraria montana |
| ispS | CAC35696. 1 | 13539551 | Populus tremula × Populus alba |

### 3-Hydroxyglutaryl-CoA Reductase (Alcohol Forming) (FIG. 12, Step J)

This step catalyzes the reduction of the acyl-CoA group in 3-hydroxyglutaryl-CoA to the alcohol group. Exemplary bifunctional oxidoreductases that convert an acyl-CoA to alcohol include those that transform substrates such as acetyl-CoA to ethanol (e.g., adhE from E. coli (Kessler et al., supra, (1991)) and butyryl-CoA to butanol (e.g. adhE2 from C. acetobutylicum (Fontaine et al., supra, (2002)). In addition to reducing acetyl-CoA to ethanol, the enzyme encoded by adhE in Leuconostoc mesenteroides has been shown to oxide the branched chain compound isobutyraldehyde to isobutyryl-CoA (Kazahaya et al., supra, (1972); Koo et al., supra, (2005)).

Another exemplary enzyme can convert malonyl-CoA to 3-HP. An NADPH-dependent enzyme with this activity has characterized in Chloroflexus aurantiacus where it participates in the 3-hydroxypropionate cycle (Hugler et al., supra, (2002); Strauss and Fuchs, supra, (1993)). This enzyme, with a mass of 300 kDa, is highly substrate-specific and shows little sequence similarity to other known oxidoreductases (Hugler et al., supra, (2002)). No enzymes in other organisms have been shown to catalyze this specific reaction; however there is bioinformatic evidence that other organisms can have similar pathways (Klatt et al., supra, (2007)). Enzyme candidates in other organisms including Roseillexus castenholzii, Erythrobacter sp. NAP1 and marine gamma proteobacterium HTCC2080 can be inferred by sequence similarity.

| Protein | GenBank ID | GI Number | Organism |
|---|---|---|---|
| adhE | NP_415757.1 | 16129202 | Escherichia coli |
| adhE2 | AAK09379.1 | 12958626 | Clostridium acetobutylicum |
| adhE | AAV66076.1 | 55818563 | Leuconostoc mesenteroides |
| mcr | AAS20429.1 | 42561982 | Chloroflexus aurantiacus |
| Rcas_2929 | YP_001433009.1 | 156742880 | Roseiflexus castenholzii |
| NAP1_02720 | ZP_01039179.1 | 85708113 | Erythrobacter sp. NAP1 |
| MGP2080_00535 | ZP_01626393.1 | 119504313 | marine gamma proteobacterium HTCC2080 |

Longer chain acyl-CoA molecules can be reduced to their corresponding alcohols by enzymes such as the jojoba (Simmondsia chinensis) FAR which encodes an alcohol-forming fatty acyl-CoA reductase. Its overexpression in E. coli resulted in FAR activity and the accumulation of fatty alcohol (Metz et al., Plant Physiology 122:635-644 (2000)).

| Protein | GenBank ID | GI Number | Organism |
|---|---|---|---|
| FAR | AAD38039.1 | 5020215 | Simmondsia chinensis |

Another candidate for catalyzing this step is 3-hydroxy-3-methylglutaryl-CoA reductase (or HMG-CoA reductase). This enzyme reduces the CoA group in 3-hydroxy-3-methylglutaryl-CoA to an alcohol forming mevalonate. Gene candidates for this step include:

| Protein | GenBank ID | GI Number | Organism |
|---|---|---|---|
| HMG1 | CAA86503.1 | 587536 | Saccharomyces cerevisiae |
| HMG2 | NP_013555 | 6323483 | Saccharomyces cerevisiae |
| HMG1 | CAA70691.1 | 1694976 | Arabidopsis thaliana |
| hmgA | AAC45370.1 | 2130564 | Sulfolobus solfataricus |

The hmgA gene of Sulfolobus solfataricus, encoding 3-hydroxy-3-methylglutaryl-CoA reductase, has been cloned, sequenced, and expressed in E. coli (Bochar et al., J Bacteriol. 179:3632-3638 (1997)). S. cerevisiae also has two HMG-CoA reductases in it (Basson et al., Proc. Natl. Acad. Sci. U.S.A 83:5563-5567 (1986)). The gene has also been isolated from Arabidopsis thaliana and has been shown to complement the HMG-COA reductase activity in S. cerevisiae (Learned et al., Proc. Natl. Acad. Sci. U.S.A 86:2779-2783 (1989)).

### 3-Oxoglutaryl-CoA Reductase (Aldehyde Forming) (FIG. 12, Step K)

Several acyl-CoA dehydrogenases are capable of reducing an acyl-CoA to its corresponding aldehyde. Thus they can naturally reduce 3-oxoglutaryl-CoA to 3,5-dioxopentanoate or can be engineered to do so. Exemplary genes that encode such enzymes were discussed in FIG. 12, Step C.

### 3,5-Dioxopentanoate Reductase (Ketone Reducing) (FIG. 12, Step L)

There exist several exemplary alcohol dehydrogenases that convert a ketone to a hydroxyl functional group. Two such enzymes from E. coli are encoded by malate dehydrogenase (mdh) and lactate dehydrogenase (ldhA). In addition, lactate dehydrogenase from Ralstonia eutropha has been shown to demonstrate high activities on 2-ketoacids of various chain lengths including lactate, 2-oxobutyrate, 2-oxopentanoate and 2-oxoglutarate (Steinbuchel et al., Eur. J. Biochem. 130:329-334 (1983)). Conversion of alpha-ketoadipate into alpha-hydroxyadipate can be catalyzed by 2-ketoadipate reductase, an enzyme reported to be found in rat and in human placenta (Suda et al., Arch. Biochem. Biophys. 176:610-620 (1976); Suda et al., Biochem. Biophys. Res. Commun. 77:586-591 (1977)). An additional candidate for this step is the mitochondrial 3-hydroxybutyrate dehydrogenase (bdh) from the human heart which has been cloned and characterized (Marks et al., J. Biol. Chem. 267:15459-15463 (1992)). This enzyme is a dehydrogenase that operates on a 3-hydroxyacid. Another exemplary alcohol dehydrogenase converts acetone to isopropanol as was shown in C. beijerinckii (Ismaiel et al., J. Bacteriol. 175:5097-5105 (1993)) and T. brockii (Lamed et al., Biochem. J. 195:183-190 (1981); Peretz et al., Biochemistry. 28:6549-6555 (1989)). Methyl ethyl ketone reductase, or alternatively, 2-butanol dehydrogenase, catalyzes the reduction of MEK to form 2-butanol. Exemplary enzymes can be found in Rhodococcus ruber (Kosjek et al., Biotechnol Bioeng. 86:55-62 (2004)) and Pyrococcus furiosus (van der et al., Eur. J. Biochem. 268:3062-3068 (2001)).

| Protein | GenBank ID | GI Number | Organism |
|---|---|---|---|
| mdh | AAC76268.1 | 1789632 | Escherichia coli |
| ldhA | NP_415898. 1 | 16129341 | Escherichia coli |
| ldh | YP_725182.1 | 113866693 | Ralstonia eutropha |
| bdh | AAA 58352.1 | 177198 | Homo sapiens |
| adh | AAA23199.2 | 60592974 | Clostridium beijerinckii NRRL B593 |
| adh | P14941.1 | 113443 | Thermoanaerobacter brockii HTD4 |
| adhA | AAC25556 | 3288810 | Pyrococcus furiosus |
| adh-A | CAD36475 | 21615553 | Rhodococcus ruber |

A number of organisms can catalyze the reduction of 4-hydroxy-2-butanone to 1,3-butanediol, including those belonging to the genus Bacillus, Brevibacterium, Candida, and Klebsiella among others, as described by Matsuyama et al. U.S. Pat. No. 5,413,922. A mutated Rhodococcus phenylacetaldehyde reductase (Sar268) and a Leifonia alcohol dehydrogenase have also been shown to catalyze this transformation at high yields (Itoh et al., Appl. Microbiol. Biotechnol. 75(6):1249-1256).

Homoserine dehydrogenase (EC 1.1.1.13) catalyzes the NAD(P)H-dependent reduction of aspartate semialdehyde to homoserine. In many organisms, including E. coli, homoserine dehydrogenase is a bifunctional enzyme that also catalyzes the ATP-dependent conversion of aspartate to aspartyl-4-phosphate (Starnes et al., Biochemistry 11:677-687 (1972)). The functional domains are catalytically independent and connected by a linker region (Sibilli et al., J Biol Chem 256:10228-10230 (1981)) and both domains are subject to allosteric inhibition by threonine. The homoserine dehydrogenase domain of the E. coli enzyme, encoded by thrA, was separated from the aspartate kinase domain, characterized, and found to exhibit high catalytic activity and reduced inhibition by threonine (James et al., Biochemistry 41:3720-3725 (2002)). This can be applied to other bifunctional threonine kinases including, for example, hom1 of Lactobacillus plantarum (Cahyanto et al., Microbiology 152:105-112 (2006)) and Arabidopsis thaliana. The monofunctional homoserine dehydrogenases encoded by hom6 in S. cerevisiae (Jacques et al., Biochim Biophys Acta 1544:28-41 (2001)) and hom2 in Lactobacillus plantarum (Cahyanto et al., supra, (2006)) have been functionally expressed and characterized in E. coli.

| Protein | GenBank ID | GI number | Organism |
|---|---|---|---|
| thrA | AAC73113.1 | 1786183 | Escherichia coli K12 |
| akthr2 | O81852 | 75100442 | Arabidopsis thaliana |
| hom6 | CAA89671 | 1015880 | Saccharomyces cerevisiae |
| hom1 | CAD 64819 | 28271914 | Lactobacillus plantarum |
| hom2 | CAD 63186 | 28270285 | Lactobacillus plantarum |

### 3,5-Dioxopentanoate Reductase (Aldehyde Reducing) (FIG. 12, Step M)

Several aldehyde reducing reductases are capable of reducing an aldehyde to its corresponding alcohol. Thus they can naturally reduce 3,5-dioxopentanoate to 5-hydroxy-3-oxopentanoate or can be engineered to do so. Exemplary genes that encode such enzymes were discussed in FIG. 12, Step D.

### 5-Hydroxy-3-Oxopentanoate Reductase (FIG. 12, Step N)

Several ketone reducing reductases are capable of reducing a ketone to its corresponding hydroxyl group. Thus they can naturally reduce 5-hydroxy-3-oxopentanoate to 3,5-dihydroxypentanoate or can be engineered to do so. Exemplary genes that encode such enzymes were discussed in FIG. 12, Step L.

### 3-Oxo-Glutaryl-CoA Reductase (CoA Reducing and Alcohol Forming) (FIG. 12, Step O)

3-oxo-glutaryl-CoA reductase (CoA reducing and alcohol forming) enzymes catalyze the 2 reduction steps required to form 5-hydroxy-3-oxopentanoate from 3-oxo-glutaryl-CoA. Exemplary 2-step oxidoreductases that convert an acyl-CoA to an alcohol were provided for FIG. 12, Step J. Such enzymes can naturally convert 3-oxo-glutaryl-CoA to 5-hydroxy-3-oxopentanoate or can be engineered to do so.

### Example VII

### Pathways for Converting Pyruvate to 2-Butanol, and 2-Butanol to 3-Butene-2-ol

This example describes an enzymatic pathway for converting pyruvate to 2-butanol, and further to 3-buten-2-ol. The 3-buten-2-ol product can be isolated as the product, or further converted to 1,3-butadiene via enzymatic or chemical dehydration. Chemical dehydration of 3-buten-2-ol to butadiene is well known in the art (Gustay. Egloff and George. Hulla, Chem. Rev., 1945, 36 (1), pp 63-141).

Pathways for converting pyruvate to 2-butanol are well known in the art and are incorporated herein by reference (U.S. Pat. No. 8,206,970, WO 2010/057022). One exemplary pathway for converting pyruvate to 2-butanol is shown in FIG. 14. In this pathway, acetolactate is formed from pyruvate by acetolactate synthase (Step A), acetolactate is subsequently decarboxylated to acetoin by acetolactate decarboxylase (step B). Reduction of acetoin to 2,3-butanediol and subsequent dehydration (Steps 2C-D) yield 2-butanol. Exemplary enzymes for steps A-D are listed in the table below.

| Step | Gene | GenBank ID | GI Number | Organism |
|---|---|---|---|---|
| 14A | budB | AAA25079 | 149211 | Klebsiella pneumonia |
| | | | | ATCC 25955 |
| 14A | alsS | AAA22222 | 142470 | Bacillus subtilis |
| 14A | budB | AAA25055 | 149172 | Klebsiella terrigena |
| 14B | budA | AAU43774 | 52352568 | Klebsiella oxytoca |
| 14B | alsD | AAA22223 | 142471 | Bacillus subtilis |
| 14B | budA | AAA25054 | 149171 | Klebsiella terrigena |
| 14C | sadH | CAD36475 | 21615553 | Rhodococcus ruber |
| 14C | budC | D86412.1 | 1468938 | Klebsiella pneumonia |
| | | | | IAM1063 |
| 14C | BC_0668 | AAP07682 | 29894392 | Bacillus cereus |
| 14C | butB | AAK04995 | 12723828 | Lactococcus lactis |
| 14D | pddC | AAC98386.1 | 4063704 | Klebsiella pneumoniae |
| 14D | pddB | AAC98385.1 | 4063703 | Klebsiella pneumoniae |
| 14D | pddA | AAC98384.1 | 4063702 | Klebsiella pneumoniae |
| 14D | pduC | AAB84102.1 | 2587029 | Salmonella typhimurium |
| 14D | pduD | AAB84103.1 | 2587030 | Salmonella typhimurium |
| 14D | pduE | AAB84104.1 | 2587031 | Salmonella typhimurium |
| 14D | pddA | BAA08099.1 | 868006 | Klebsiella oxytoca |
| 14D | pddB | BAA08100.1 | 868007 | Klebsiella oxytoca |
| 14D | pddC | BAA08101.1 | 868008 | Klebsiella oxytoca |
| 14D | pduC | CAC82541.1 | 18857678 | Lactobacillus collinoides |
| 14D | pduD | CAC82542.1 | 18857679 | Lactobacillus collinoides |
| 14D | pduE | CAD01091.1 | 18857680 | Lactobacillus collinoides |

Enzyme candidates for steps 13A and 13B are disclosed below.

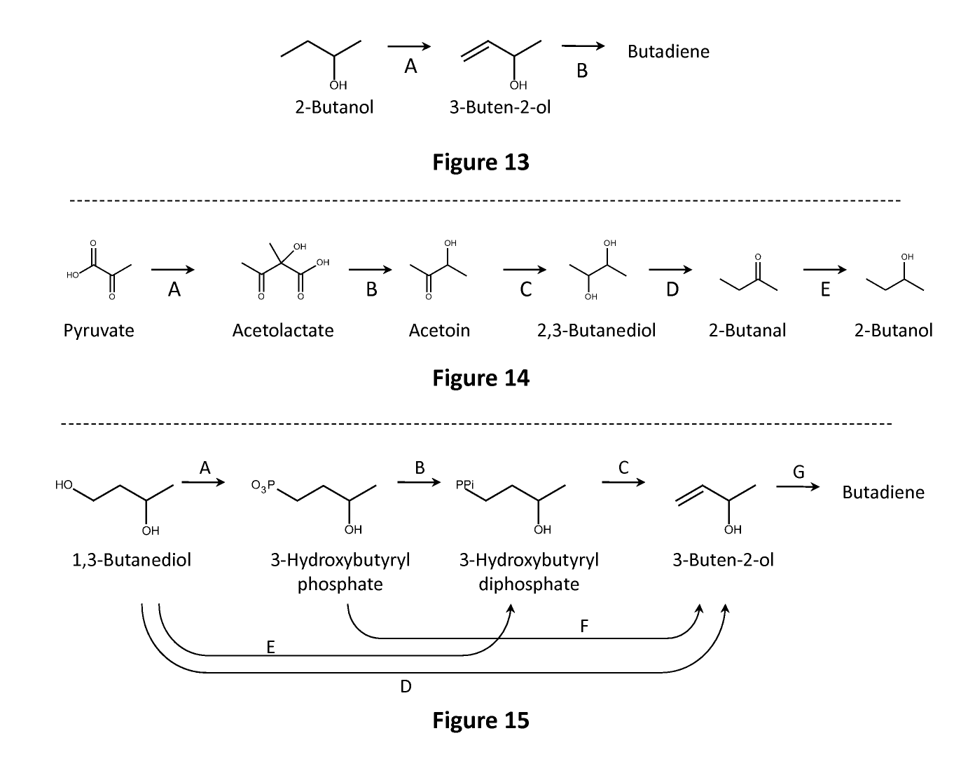

### 2-Butanol Desaturase (FIG. 13A)

Conversion of 2-butanol to 3-buten-2-ol is catalyzed by an enzyme with 2-butanol desaturase activity (Step 1A). An exemplary enzyme is MdpJ from Aquincola tertiaricarbonis L108 (Schaefer et al, AEM 78 (17): 6280-4 (2012); Schuster et al, J. Bacteriol 194:972-81 (2012)). This enzyme is a Rieske non-heme mononuclear iron oxygenase, a class of enzymes which typically reacts with aromatic substrates. The MdpJ gene product is active on aliphatic secondary and tertiary alcohol substrates including 2-butanol, 3-methyl-2-butanol and 3-pentanol. The net reaction of MdpJ is conversion of 2-butanol, oxygen and NADH to 3-buten-2-ol, NAD and water. The MdpJ gene is colocalized in an operon with several genes that may encode accessory proteins required for activity, listed in the table below. A similar enzyme is found in M. petroleiphilum PM1 (Schuster et al, supra). The mdpK gene encodes a ferredoxin oxidoreductase that may be required for mdpJ activation (Hristova et al, AEM 73: 7347-57 (2007)). Other enzyme candidates can be identified by sequence similarity and are shown in the table below.

| Protein | GenBank ID | GI Number | Organism |
|---|---|---|---|
| mdpJ | AEX20406 | 369794441 | Aquincola tertiaricarbonis L108 |
| mdpK | AEX20407 | 369794442 | Aquincola tertiaricarbonis L108 |
| JQ062962.1:4013..4777 | AEX20409 | 369794444 | Aquincola tertiaricarbonis L108 |
| JQ062962.1:4796..5074 | AEX20408 | 369794443 | Aquincola tertiaricarbonis L108 |
| JQ062962.1:5190..6062 | AEX20410 | 369794445 | Aquincola tertiaricarbonis L108 |
| mdpJ | YP_001023560.1 | 124263090 | Methylibium petroleiphilum PM1 |
| mdpK | YP_001023559.1 | 124263089 | Methylibium petroleiphilum PM1 |
| Mpe_B0553 | YP_001023558.1 | 124263088 | Methylibium petroleiphilum PM1 |
| Mpe_B0552 | YP_001023557.1 | 124263087 | Methylibium petroleiphilum PM1 |
| Mpe_B0551 | YP_001023556.1 | 124263086 | Methylibium petroleiphilum PM1 |
| BN115_3999 | YP_006902223.1 | 410421774 | Bordetella bronchiseptica MO149 |
| NC_002928.3:4169127..4170563 | NP_886002.1 | 33598359 | Bordetella parapertussis 12822 |
| NZ_GL982453.1:6380824..6382248 | ZP_17009234 | NZ_AFRQ01000000 | Achromobacter xylosoxidans AXX- A |

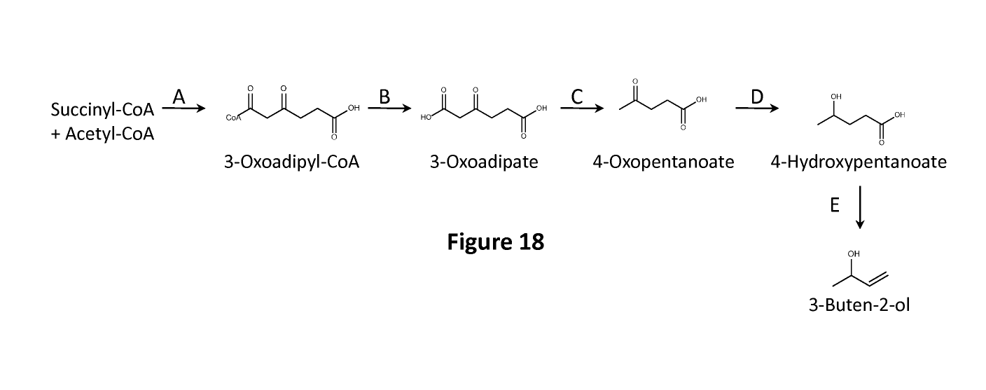

### 3-Buten-2-ol Dehydratase (FIG. 13B—Also Applicable to Step G of FIG. 15, Step E of 16, Step G of FIG. 17, and Step F of FIG. 18)

Dehydration of 3-buten-2-ol to butadiene is catalyzed by a 3-buten-2-ol dehydratase enzyme (Step 13B) or by chemical dehydration. Exemplary dehydratase enzymes suitable for dehydrating 3-buten-2-ol include oleate hydratase, acyclic 1,2-hydratase and linalool dehydratase enzymes. Oleate hydratases catalyze the reversible hydration of non-activated alkenes to their corresponding alcohols. Oleate hydratase enzymes disclosed in WO2011/076691 and WO 2008/119735 are incorporated by reference herein. Oleate hydratases from Elizabethkingia meningoseptica and Streptococcus pyogenes are encoded by ohyA and HMPREF0841_{—}1446. Acyclic 1,2-hydratase enzymes (eg. EC 4.2.1.131) catalyze the dehydration of linear secondary alcohols, and are thus suitable candidates for the dehydration of 3-buten-2-ol to butadiene. Exemplary 1,2-hydratase enzymes include carotenoid 1,2-hydratase, encoded by crtC of Rubrivivax gelatinosus (Steiger et al, Arch Biochem Biophys 414:51-8 (2003)), and lycopene 1,2-hydratase, encoded by cruF of Synechococcus sp. PCC 7002 and Gemmatimonas aurantiaca (Graham and Bryant, J Bacteriol 191: 2392-300 (2009); Takaichi et al, Microbiol 156: 756-63 (2010)). Dehydration of t-butyl alcohol, t-amyl alcohol and 2-methyl-3-buten-2-ol to isobutene, isoamylene and isoprene, respectively, is catalyzed by an unknown enzyme of Aquincola tertiaricarbonis L108 (Schaefer et al, AEM 78 (17): 6280-4 (2012); Schuster et al, J. Bacteriol 194:972-81 (2012); Schuster et al, J Bacteriol 194: 972-81 (2012)). This dehydratase enzyme is also a suitable enzyme candidate for dehydrating 3-buten-2-ol to butadiene. The linalool dehydratase/isomerase of Castellaniella defragrans catalyzes the dehydration of linalool to myrcene, reactants similar in structure to 3-buten-2-ol and butadiene (Brodkorb et al, J Biol Chem 285:30436-42 (2010)). Enzyme accession numbers and homologs are listed in the table below.

| Protein | GenBank ID | GI Number | Organism |
|---|---|---|---|
| OhyA | ACT54545.1 | 254031735 | Elizabethkingia meningoseptica |
| HMPREF0841_1446 | ZP_07461147.1 | 306827879 | Streptococcus pyogenes ATCC 10782 |
| P700755_13397 | ZP_01252267.1 | 91215295 | Psychroflexus torquis ATCC 700755 |
| RPB_2430 | YP_486046.1 | 86749550 | Rhodopseudomonas palustris |
| CrtC | AAO93124.1 | 29893494 | Rubrivivax gelatinosus |
| CruF | YP_001735274.1 | 170078636 | Synechococcus sp. PCC 7002 |
| Ldi | E1XUJ2.1 | 403399445 | Castellaniella defragrans |
| STEHIDRAFT_68678 | EIM80109.1 | 389738914 | Stereum hirsutum FP-91666 SS1 |
| NECHADRAFT_82460 | XP_003040778.1 | 302883759 | Nectria haematococca mpVI 77-13-4 |
| AS9A_2751 | YP_004493998.1 | 333920417 | Amycolicicoccus subflavus DQS3-9A1 |

### Example VIII

### Pathway for Converting 1,3-Butanediol to 3-Buten-2-Ol and/or Butadiene

FIG. 15 shows pathways for converting 1,3-butanediol to 3-buten-2-ol and/or butadiene. Enzymes in FIG. 15 are A. 1,3-butanediol kinase, B. 3-hydroxybutyrylphosphate kinase, C. 3-hydroxybutyryldiphosphate lyase, D. 1,3-butanediol diphosphokinase, E. 1,3-butanediol dehydratase, F. 3-hydroxybutyrylphosphate lyase, G. 3-buten-2-ol dehydratase or chemical reaction.

Enzyme candidates for catalyzing steps A, B, C, E and F of FIG. 15 are described below. Enzymes for step G are described above.

### 1,3-Butanediol Kinase (FIG. 15, Step A)

Phosphorylation of 1,3-butanediol to 3-hydroxybutyrylphosphate is catalyzed by an alcohol kinase enzyme. Alcohol kinase enzymes catalyze the transfer of a phosphate group to a hydroxyl group. Kinases that catalyze transfer of a phosphate group to an alcohol group are members of the EC 2.7.1 enzyme class. The table below lists several useful kinase enzymes in the EC 2.7.1 enzyme class.

| Enzyme | |
|---|---|
| Commission | |
| Number | Enzyme Name |
| 2.7.1.1 | hexokinase |
| 2.7.1.2 | glucokinase |
| 2.7.1.3 | ketohexokinase |
| 2.7.1.4 | fructokinase |
| 2.7.1.5 | rhamnulokinase |
| 2.7.1.6 | galactokinase |
| 2.7.1.7 | mannokinase |
| 2.7.1.8 | glucosamine kinase |
| 2.7.1.10 | phosphoglucokinase |
| 2.7.1.11 | 6-phosphofructokinase |
| 2.7.1.12 | gluconokinase |
| 2.7.1.13 | dehydrogluconokinase |
| 2.7.1.14 | sedoheptulokinase |
| 2.7.1.15 | ribokinase |
| 2.7.1.16 | ribulokinase |
| 2.7.1.17 | xylulokinase |
| 2.7.1.18 | phosphoribokinase |
| 2.7.1.19 | phosphoribulokinase |
| 2.7.1.20 | adenosine kinase |
| 2.7.1.21 | thymidine kinase |
| 2.7.1.22 | ribosylnicotinamide kinase |
| 2.7.1.23 | NAD+ kinase |
| 2.7.1.24 | dephospho-CoA kinase |
| 2.7.1.25 | adenylyl-sulfate kinase |
| 2.7.1.26 | riboflavin kinase |
| 2.7.1.27 | erythritol kinase |
| 2.7.1.28 | triokinase |
| 2.7.1.29 | glycerone kinase |
| 2.7.1.30 | glycerol kinase |
| 2.7.1.31 | glycerate kinase |
| 2.7.1.32 | choline kinase |
| 2.7.1.33 | pantothenate kinase |
| 2.7.1.34 | pantetheine kinase |
| 2.7.1.35 | pyridoxal kinase |
| 2.7.1.36 | mevalonate kinase |
| 2.7.1.39 | homoserine kinase |
| 2.7.1.40 | pyruvate kinase |
| 2.7.1.41 | glucose-1-phosphate |
| | phosphodismutase |
| 2.7.1.42 | riboflavin |
| | phosphotransferase |
| 2.7.1.43 | glucuronokinase |
| 2.7.1.44 | galacturonokinase |
| 2.7.1.45 | 2-dehydro-3- |
| | deoxygluconokinase |
| 2.7.1.46 | L-arabinokinase |
| 2.7.1.47 | D-ribulokinase |
| 2.7.1.48 | uridine kinase |
| 2.7.1.49 | hydroxymethylpyrimidine kinase |
| 2.7.1.50 | hydroxyethylthiazole kinase |
| 2.7.1.51 | L-fuculokinase |
| 2.7.1.52 | fucokinase |
| 2.7.1.53 | L-xylulokinase |
| 2.7.1.54 | D-arabinokinase |
| 2.7.1.55 | allose kinase |
| 2.7.1.56 | 1-phosphofructokinase |
| 2.7.1.58 | 2-dehydro-3-deoxygalactonokinase |
| 2.7.1.59 | N-acetylglucosamine kinase |
| 2.7.1.60 | N-acylmannosamine kinase |
| 2.7.1.61 | acyl-phosphate—hexose |
| | phosphotransferase |
| 2.7.1.62 | phosphoramidate—hexose |
| | phosphotransferase |
| 2.7.1.63 | polyphosphate—glucose |
| | phosphotransferase |
| 2.7.1.64 | inositol 3-kinase |
| 2.7.1.65 | scyllo-inosamine 4-kinase |
| 2.7.1.66 | undecaprenol kinase |
| 2.7.1.67 | 1-phosphatidylinositol 4-kinase |
| 2.7.1.68 | 1-phosphatidylinositol-4-phosphate |
| | 5-kinase |
| 2.7.1.69 | protein-Np-phosphohistidine— |
| | sugar phosphotransferase |
| 2.7.1.70 | identical to EC 2.7.1.37. |
| 2.7.1.71 | shikimate kinase |
| 2.7.1.72 | streptomycin 6-kinase |
| 2.7.1.73 | inosine kinase |
| 2.7.1.74 | deoxycytidine kinase |
| 2.7.1.76 | deoxyadenosine kinase |
| 2.7.1.77 | nucleoside phosphotransferase |
| 2.7.1.78 | polynucleotide 5′-hydroxyl-kinase |
| 2.7.1.79 | diphosphate—glycerol |
| | phosphotransferase |
| 2.7.1.80 | diphosphate—serine |
| | phosphotransferase |
| 2.7.1.81 | hydroxylysine kinase |
| 2.7.1.82 | ethanolamine kinase |
| 2.7.1.83 | pseudouridine kinase |
| 2.7.1.84 | alkylglycerone kinase |
| 2.7.1.85 | β-glucoside kinase |
| 2.7.1.86 | NADH kinase |
| 2.7.1.87 | streptomycin 3″-kinase |
| 2.7.1.88 | dihydrostreptomycin-6-phosphate |
| | 3'a-kinase |
| 2.7.1.89 | thiamine kinase |
| 2.7.1.90 | diphosphate—fructose-6- |
| | phosphate 1-phosphotransferase |
| 2.7.1.91 | sphinganine kinase |
| 2.7.1.92 | 5-dehydro-2-deoxygluconokinase |
| 2.7.1.93 | alkylglycerol kinase |
| 2.7.1.94 | acylglycerol kinase |
| 2.7.1.95 | kanamycin kinase |
| 2.7.1.100 | S-methyl-5-thioribose kinase |
| 2.7.1.101 | tagatose kinase |
| 2.7.1.102 | hamamelose kinase |
| 2.7.1.103 | viomycin kinase |
| 2.7.1.105 | 6-phosphofructo-2-kinase |
| 2.7.1.106 | glucose-1,6-bisphosphate |
| | synthase |
| 2.7.1.107 | diacylglycerol kinase |
| 2.7.1.108 | dolichol kinase |
| 2.7.1.113 | deoxyguanosine kinase |
| 2.7.1.114 | AMP—thymidine kinase |
| 2.7.1.118 | ADP—thymidine kinase |
| 2.7.1.119 | hygromycin-B 7″-O-kinase |
| 2.7.1.121 | phosphoenolpyruvate—glycerone |
| | phosphotransferase |
| 2.7.1.122 | xylitol kinase |
| 2.7.1.127 | inositol-trisphosphate 3-kinase |
| 2.7.1.130 | tetraacyldisaccharide 4′-kinase |
| 2.7.1.134 | inositol-tetrakisphosphate 1- |
| | kinase |
| 2.7.1.136 | macrolide 2′-kinase |
| 2.7.1.137 | phosphatidylinositol 3-kinase |
| 2.7.1.138 | ceramide kinase |
| 2.7.1.140 | inositol-tetrakisphosphate 5- |
| | kinase |
| 2.7.1.142 | glycerol—3-phosphate-glucose |
| | phosphotransferase |
| 2.7.1.143 | diphosphate-purine nucleoside |
| | kinase |
| 2.7.1.144 | tagatose-6-phosphate kinase |
| 2.7.1.145 | deoxynucleoside kinase |
| 2.7.1.146 | ADP-dependent |
| | phosphofructokinase |
| 2.7.1.147 | ADP-dependent glucokinase |
| 2.7.1.148 | 4-(cytidine 5′-diphospho)-2-C- |
| | methyl-D-erythritol kinase |
| 2.7.1.149 | 1-phosphatidylinositol-5- |
| | phosphate 4-kinase |
| 2.7.1.150 | 1-phosphatidylinositol-3- |
| | phosphate 5-kinase |
| 2.7.1.151 | inositol-polyphosphate |
| | multikinase |
| 2.7.1.153 | phosphatidylinositol-4,5- |
| | bisphosphate 3-kinase |
| 2.7.1.154 | phosphatidylinositol-4-phosphate |
| | 3-kinase |
| 2.7.1.156 | adenosylcobinamide kinase |
| 2.7.1.157 | N-acetylgalactosamine kinase |
| 2.7.1.158 | inositol-pentakisphosphate 2- |
| | kinase |
| 2.7.1.159 | inositol-1,3,4-trisphosphate 5/6- |
| | kinase |
| 2.7.1.160 | 2′-phosphotransferase |
| 2.7.1.161 | CTP-dependent riboflavin kinase |
| 2.7.1.162 | N-acetylhexosamine 1-kinase |
| 2.7.1.163 | hygromycin B 4-O-kinase |
| 2.7.1.164 | O-phosphoseryl-tRNASec kinase |

Mevalonate kinase (EC 2.7.1.36) phosphorylates the terminal hydroxyl group of mevalonate. Gene candidates for this step include erg12 from S. cerevisiae, mvk from Methanocaldococcus jannaschi, MVK from Homo sapeins, and mvk from Arabidopsis thaliana col. Additional mevalonate kinase candidates include the feedback-resistant mevalonate kinase from the archeon Methanosarcina mazei (Primak et al, AEM, in press (2011)) and the Mvk protein from Streptococcus pneumoniae (Andreassi et al, Protein Sci, 16:983-9 (2007)). Mvk proteins from S. cerevisiae, S. pneumoniae and M. mazei were heterologously expressed and characterized in E. coli (Primak et al, supra). The S. pneumoniae mevalonate kinase was active on several alternate substrates including cylopropylmevalonate, vinylmevalonate and ethynylmevalonate (Kudoh et al, Bioorg Med Chem 18:1124-34 (2010)), and a subsequent study determined that the ligand binding site is selective for compact, electron-rich C(3)-substituents (Lefurgy et al, J Biol Chem 285:20654-63 (2010)).

| Protein | GenBank ID | GI Number | Organism |
|---|---|---|---|
| erg12 | CAA39359.1 | 3684 | Sachharomyces cerevisiae |
| mvk | Q58487.1 | 2497517 | Methanocaldococcus jannaschii |
| mvk | AAH16140.1 | 16359371 | Homo sapiens |
| mvk | NP_851084.1 | 30690651 | Arabidopsis thaliana |
| mvk | NP_633786.1 | 21227864 | Methanosarcina mazei |
| mvk | NP_357932.1 | 15902382 | Streptococcus pneumoniae |

Glycerol kinase also phosphorylates the terminal hydroxyl group in glycerol to form glycerol-3-phosphate. This reaction occurs in several species, including Escherichia coli, Saccharomyces cerevisiae, and Thermotoga maritima. The E. coli glycerol kinase has been shown to accept alternate substrates such as dihydroxyacetone and glyceraldehyde (Hayashi et al., J Biol. Chem. 242:1030-1035 (1967)). T. maritime has two glycerol kinases (Nelson et al., Nature 399:323-329 (1999)). Glycerol kinases have been shown to have a wide range of substrate specificity. Crans and Whiteside studied glycerol kinases from four different organisms (Escherichia coli, S. cerevisiae, Bacillus stearothermophilus, and Candida mycoderma) (Crans et al., J. Am. Chem. Soc. 107:7008-7018 (2010); Nelson et al., supra, (1999)). They studied 66 different analogs of glycerol and concluded that the enzyme could accept a range of substituents in place of one terminal hydroxyl group and that the hydrogen atom at C2 could be replaced by a methyl group. Interestingly, the kinetic constants of the enzyme from all four organisms were very similar

| Protein | GenBank ID | GI Number | Organism |
|---|---|---|---|
| glpK | AP_003883.1 | 89110103 | Escherichia coli K12 |
| glpK1 | NP_228760.1 | 15642775 | Thermotoga maritime MSB8 |
| glpK2 | NP_229230.1 | 15642775 | Thermotoga maritime MSB8 |
| Gut1 | NP_011831.1 | 82795252 | Saccharomyces cerevisiae |

Homoserine kinase is another similar enzyme candidate. This enzyme is also present in a number of organisms including E. coli, Streptomyces sp, and S. cerevisiae. Homoserine kinase from E. coli has been shown to have activity on numerous substrates, including, L-2-amino, 1,4-butanediol, aspartate semialdehyde, and 2-amino-5-hydroxyvalerate (Huo et al., Biochemistry 35:16180-16185 (1996); Huo et al., Arch. Biochem. Biophys. 330:373-379 (1996)). This enzyme can act on substrates where the carboxyl group at the alpha position has been replaced by an ester or by a hydroxymethyl group. The gene candidates are:

| Protein | GenBank ID | GI Number | Organism |
|---|---|---|---|
| thrB | BAB96580.2 | 85674277 | Escherichia coli |
| | | | K12 |
| SACT1DRAFT_4809 | ZP_06280784.1 | 282871792 | Streptomyces sp. |
| | | | ACT-1 |
| Thr1 | AAA35154.1 | 172978 | Saccharomyces |
| | | | serevisiae |

### 3-Hydroxybutyrylphosphate Kinase (FIG. 15, Step B)

Alkyl phosphate kinase enzymes catalyze the transfer of a phosphate group to the phosphate group of an alkyl phosphate. The enzymes described below naturally possess such activity or can be engineered to exhibit this activity. Kinases that catalyze transfer of a phosphate group to another phosphate group are members of the EC 2.7.4 enzyme class. The table below lists several useful kinase enzymes in the EC 2.7.4 enzyme class.

| Enzyme | |
|---|---|
| Commission No. | Enzyme Name |
| 2.7.4.1 | polyphosphate kinase |
| 2.7.4.2 | phosphomevalonate kinase |
| 2.7.4.3 | adenylate kinase |
| 2.7.4.4 | nucleoside-phosphate kinase |
| 2.7.4.6 | nucleoside-diphosphate kinase |
| 2.7.4.7 | phosphomethylpyrimidine kinase |
| 2.7.4.8 | guanylate kinase |
| 2.7.4.9 | dTMP kinase |
| 2.7.4.10 | nucleoside-triphosphate—adenylate kinase |
| 2.7.4.11 | (deoxy)adenylate kinase |
| 2.7.4.12 | T2-induced deoxynucleotide kinase |
| 2.7.4.13 | (deoxy)nucleoside-phosphate kinase |
| 2.7.4.14 | cytidylate kinase |
| 2.7.4.15 | thiamine-diphosphate kinase |
| 2.7.4.16 | thiamine-phosphate kinase |
| 2.7.4.17 | 3-phosphoglyceroyl-phosphate—polyphosphate |
| | phosphotransferase |
| 2.7.4.18 | farnesyl-diphosphate kinase |
| 2.7.4.19 | 5-methyldeoxycytidine-5′-phosphate kinase |
| 2.7.4.20 | dolichyl-diphosphate—polyphosphate |
| | phosphotransferase |
| 2.7.4.21 | inositol-hexakisphosphate kinase |
| 2.7.4.22 | UMP kinase |
| 2.7.4.23 | ribose 1,5-bisphosphate phosphokinase |
| 2.7.4.24 | diphosphoinositol-pentakisphosphate kinase |
| 2.7.4.— | Farnesyl monophosphate kinase |
| 2.7.4.— | Geranyl-geranyl monophosphate kinase |
| 2.7.4.— | Phytyl-phosphate kinase |

Phosphomevalonate kinase enzymes are of particular interest. Phosphomevalonate kinase (EC 2.7.4.2) catalyzes the phosphorylation of phosphomevalonate. This enzyme is encoded by erg8 in Saccharomyces cerevisiae (Tsay et al., Mol. Cell Biol. 11:620-631 (1991)) and mvaK2 in Streptococcus pneumoniae, Staphylococcus aureus and Enterococcus faecalis (Doun et al., Protein Sci. 14:1134-1139 (2005); Wilding et al., J Bacteriol. 182:4319-4327 (2000)). The Streptococcus pneumoniae and Enterococcus faecalis enzymes were cloned and characterized in E. coli (Pilloff et al., J Biol. Chem. 278:4510-4515 (2003); Doun et al., Protein Sci. 14:1134-1139 (2005)). The S. pneumoniae phosphomevalonate kinase was active on several alternate substrates including cylopropylmevalonate phosphate, vinylmevalonate phosphate and ethynylmevalonate phosphate (Kudoh et al, Bioorg Med Chem 18:1124-34 (2010)).

| Protein | GenBank ID | GI Number | Organism |
|---|---|---|---|
| Erg8 | AAA34596.1 | 171479 | Saccharomyces cerevisiae |
| mvaK2 | AAG02426.1 | 9937366 | Staphylococcus aureus |
| mvaK2 | AAG02457.1 | 9937409 | Streptococcus pneumoniae |
| mvaK2 | AAG02442.1 | 9937388 | Enterococcus faecalis |

Farnesyl monophosphate kinase enzymes catalyze the CTP dependent phosphorylation of farnesyl monophosphate to farnesyl diphosphate. Similarly, geranylgeranyl phosphate kinase catalyzes CTP dependent phosphorylation. Enzymes with these activities were identified in the microsomal fraction of cultured Nicotiana tabacum (Thai et al, PNAS 96:13080-5 (1999)). However, the associated genes have not been identified to date.

### 3-Hydroxybutyryldiphosphate Lyase (FIG. 15, Step C)

Diphosphate lyase enzymes catalyze the conversion of alkyl diphosphates to alkenes. Carbon-oxygen lyases that operate on phosphates are found in the EC 4.2.3 enzyme class. The table below lists several useful enzymes in EC class 4.2.3. Exemplary enzyme candidates were described above (see phosphate lyase section).

| Enzyme Commission No. | Enzyme Name |
|---|---|
| 4.2.3.5 | Chorismate synthase |
| 4.2.3.15 | Myrcene synthase |
| 4.2.3.27 | Isoprene synthase |
| 4.2.3.36 | Terpentriene sythase |
| 4.2.3.46 | (E,E)-alpha-Farnesene synthase |
| 4.2.3.47 | Beta-Farnesene synthase |

### 1,3-Butanediol Dehydratase (FIG. 15, Step D)

Exemplary dehydratase enzymes suitable for dehydrating 1,3-butanediol to 3-buten-2-ol include oleate hydratases and acyclic 1,2-hydratases. Exemplary enzyme candidates are described above.

### 1,3-Butanediol Diphosphokinase (FIG. 15, Step E)

Diphosphokinase enzymes catalyze the transfer of a diphosphate group to an alcohol group. The enzymes described below naturally possess such activity. Kinases that catalyze transfer of a diphosphate group are members of the EC 2.7.6 enzyme class. The table below lists several useful kinase enzymes in the EC 2.7.6 enzyme class.

| Enzyme | |
|---|---|
| Commission No. | Enzyme Name |
| 2.7.6.1 | ribose-phosphate diphosphokinase |
| 2.7.6.2 | thiamine diphosphokinase |
| 2.7.6.3 | 2-amino-4-hydroxy-6-hydroxymethyldihydropteridine |
| | diphosphokinase |
| 2.7.6.4 | nucleotide diphosphokinase |
| 2.7.6.5 | GTP diphosphokinase |

Of particular interest are ribose-phosphate diphosphokinase enzymes, which have been identified in Escherichia coli (Hove-Jenson et al., J Biol Chem, 1986, 261(15); 6765-71) and Mycoplasma pneumoniae M129 (McElwain et al, International Journal of Systematic Bacteriology, 1988, 38:417-423) as well as thiamine diphosphokinase enzymes. Exemplary thiamine diphosphokinase enzymes are found in Arabidopsis thaliana (Ajjawi, Plant Mol Biol, 2007, 65(1-2); 151-62).

| Protein | GenBank ID | GI Number | Organism |
|---|---|---|---|
| prs | NP_415725.1 | 16129170 | Escherichia coli |
| prsA | NP_109761.1 | 13507812 | Mycoplasma pneumoniae M129 |
| TPK1 | BAH19964.1 | 222424006 | Arabidopsis thaliana col |
| TPK2 | BAH57065.1 | 227204427 | Arabidopsis thaliana col |

### 3-Hydroxybutyrylphosphate Lyase (FIG. 15, Step F)

Phosphate lyase enzymes catalyze the conversion of alkyl phosphates to alkenes. Carbon-oxygen lyases that operate on phosphates are found in the EC 4.2.3 enzyme class. The table below lists several relevant enzymes in EC class 4.2.3.

| Enzyme Commission Number | Enzyme Name |
|---|---|
| 4.2.3.5 | Chorismate synthase |
| 4.2.3.15 | Myrcene synthase |
| 4.2.3.26 | Linalool synthase |
| 4.2.3.27 | Isoprene synthase |
| 4.2.3.36 | Terpentriene sythase |
| 4.2.3.46 | (E,E)-alpha-Farnesene synthase |
| 4.2.3.47 | Beta-Farnesene synthase |
| 4.2.3.49 | Nerolidol synthase |
| 4.2.3.- | Methylbutenol synthase |

Isoprene synthase enzymes catalyzes the conversion of dimethylallyl diphosphate to isoprene. Isoprene synthases can be found in several organisms including Populus alba (Sasaki et al., FEBS Letters, 2005, 579 (11), 2514-2518), Pueraria montana (Lindberg et al., Metabolic Eng, 12(1):70-79 (2010); Sharkey et al., Plant Physiol., 137(2):700-712 (2005)), and Populus tremula×Populus alba, also called Populus canescens (Miller et al., Planta, 2001, 213 (3), 483-487). The crystal structure of the Populus canescens isoprene synthase was determined (Koksal et al, J Mol Biol 402:363-373 (2010)). Additional isoprene synthase enzymes are described in (Chotani et al., WO/2010/031079, Systems Using Cell Culture for Production of Isoprene; Cervin et al., US Patent Application 20100003716, Isoprene Synthase Variants for Improved Microbial Production of Isoprene). Another isoprene synthase-like enzyme from Pinus sabiniana, methylbutenol synthase, catalyzes the formation of 2-methyl-3-buten-2-ol (Grey et al, J Biol Chem 286: 20582-90 (2011)).

| Protein | GenBank ID | GI Number | Organism |
|---|---|---|---|
| ispS | BAD98243.1 | 63108310 | Populus alba |
| ispS | AAQ84170.1 | 35187004 | Pueraria montana |
| ispS | CAC35696.1 | 13539551 | Populus tremula x |
| | | | Populus alba |
| Tps- | AEB53064.1 | 328834891 | Pinus sabiniana |
| MBO1 | | | |

Chorismate synthase (EC 4.2.3.5) participates in the shikimate pathway, catalyzing the dephosphorylation of 5-enolpyruvylshikimate-3-phosphate to chorismate. The enzyme requires reduced flavin mononucleotide (FMN) as a cofactor, although the net reaction of the enzyme does not involve a redox change. In contrast to the enzyme found in plants and bacteria, the chorismate synthase in fungi is also able to reduce FMN at the expense of NADPH (Macheroux et al., Planta 207:325-334 (1999)). Representative monofunctional enzymes are encoded by aroC of E. coli (White et al., Biochem. J 251:313-322 (1988)) and Streptococcus pneumoniae (Maclean and Ali, Structure 11:1499-1511 (2003)). Bifunctional fungal enzymes are found in Neurospora crassa (Kitzing et al., J. Biol. Chem. 276:42658-42666 (2001)) and Saccharomyces cerevisiae (Jones et al., Mol. Microbiol. 5:2143-2152 (1991)).

| | GenBank | | |
|---|---|---|---|
| Gene | Accession No. | GI No. | Organism |
| aroC | NP_416832.1 | 16130264 | Escherichia coli |
| aroC | ACH47980.1 | 197205483 | Streptococcus |
| | | | pneumoniae |
| U25818.1:19 . . . 1317 | AAC49056.1 | 976375 | Neurospora crassa |
| ARO2 | CAA42745.1 | 3387 | Saccharomyces |
| | | | cerevisiae |

Myrcene synthase enzymes catalyze the dephosphorylation of geranyl diphosphate to beta-myrcene (EC 4.2.3.15). Exemplary myrcene synthases are encoded by MST2 of Solanum lycopersicum (van Schie et al, Plant Mol Biol 64:D473-79 (2007)), TPS-Myr of Picea abies (Martin et al, Plant Physiol 135:1908-27 (2004)) g-myr of Abies grandis (Bohlmann et al, J Biol Chem 272:21784-92 (1997)) and TPS10 of Arabidopsis thaliana (Bohlmann et al, Arch Biochem Biophys 375:261-9 (2000)). These enzymes were heterologously expressed in E. coli.

| Protein | GenBank ID | GI Number | Organism |
|---|---|---|---|
| MST2 | ACN58229.1 | 224579303 | Solanum lycopersicum |
| TPS-Myr | AAS47690.2 | 77546864 | Picea abies |
| G-myr | O24474.1 | 17367921 | Abies grandis |
| TPS10 | EC07543.1 | 330252449 | Arabidopsis thaliana |

Farnesyl diphosphate is converted to alpha-farnesene and beta-farnesene by alpha-farnesene synthase and beta-farnesene synthase, respectively. Exemplary alpha-farnesene synthase enzymes include TPS03 and TPS02 of Arabidopsis thaliana (Faldt et al, Planta 216:745-51 (2003); Huang et al, Plant Physiol 153:1293-310 (2010)), afs of Cucumis sativus (Mercke et al, Plant Physiol 135:2012-14 (2004), eafar of Malus×domestica (Green et al, Phytochem 68:176-88 (2007)) and TPS-Far of Picea abies (Martin, supra). An exemplary beta-farnesene synthase enzyme is encoded by TPS1 of Zea mays(Schnee et al, Plant Physiol 130:2049-60 (2002)).

| Protein | GenBank ID | GI Number | Organism |
|---|---|---|---|
| TPS03 | A4FVP2.1 | 205829248 | Arabidopsis thaliana |
| TPS02 | P0CJ43.1 | 317411866 | Arabidopsis thaliana |
| TPS-Far | AAS47697.1 | 44804601 | Picea abies |
| afs | AAU05951.1 | 51537953 | Cucumis sativus |
| eafar | Q84LB2.2 | 75241161 | Malus x domestica |
| TPS1 | Q84ZW8.1 | 75149279 | Zea mays |

### Example IX

### Pathways for Converting Acrylyl-CoA to 3-Butene-2-Ol and/or Butadiene

This example describes pathways for converting acrylyl-CoA to 3-buten-2-ol, and further to butadiene. The conversion of acrylyl-CoA to 3-buten-2-ol is accomplished in four enzymatic steps. Acrylyl-CoA and acetyl-CoA are first condensed to 3-oxopent-4-enoyl-CoA by 3-oxopent-4-enoyl-CoA thiolase, a beta-ketothiolase (Step 4A). The 3-oxopent-4-enoyl-CoA product is subsequently hydrolyzed to 3-oxopent-4-enoate by a CoA hydrolase, transferase or synthetase (Step 4B). Decarboxylation of the 3-ketoacid intermediate by 3-oxopent-4-enoate decarboxylase (Step 4C) yields 3-buten-2-one, which is further reduced to 3-buten-2-ol by an alcohol dehydrogenase or ketone reductase (Step 4D). 3-buten-2-ol is further converted to butadiene via chemical dehydration or by a dehydratase enzyme.

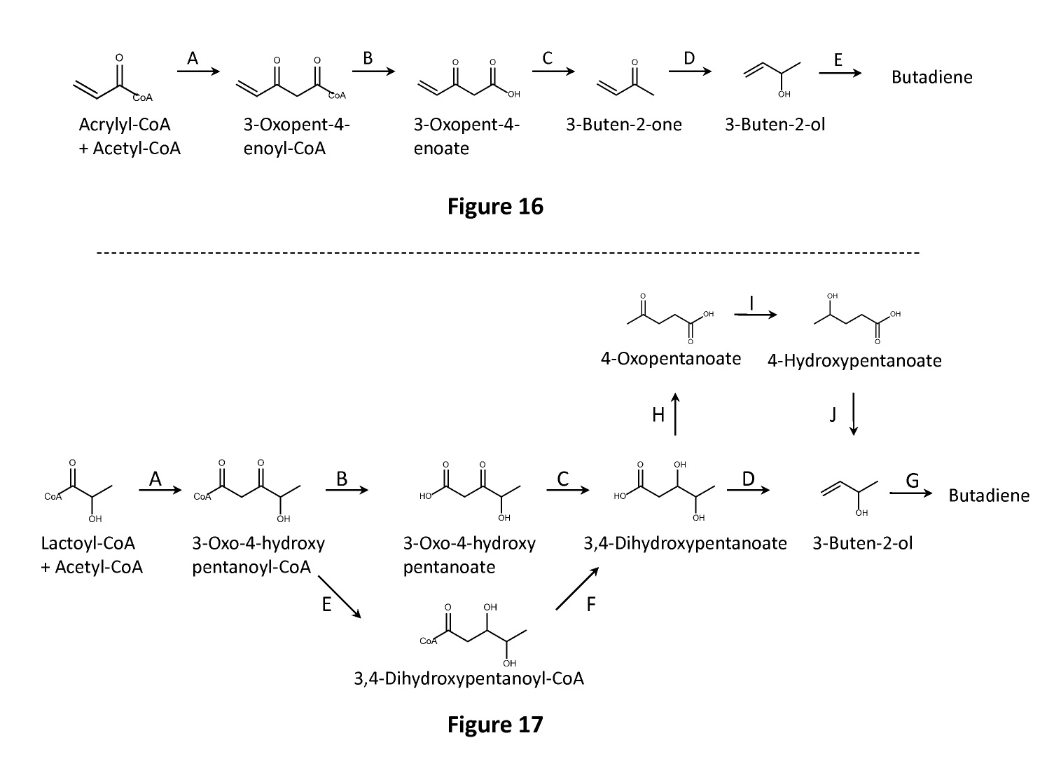

Enzymes and gene candidates for catalyzing but-3-en-2-ol and butadiene pathway reactions are described in further detail below. Enzymes for step E are described above. 3-oxopent-4-enoyl-CoA thiolase (FIG. 16, Step A)

### 3-Oxo-4-Hydroxypentanoyl-CoA Thiolase (FIG. 17, Step A)

### 3-Oxoadipyl-CoA Thiolase (FIG. 18, Step A)

Acrylyl-CoA and acetyl-CoA are condensed to form 3-oxopent-4-enoyl-CoA by a beta-ketothiolase (EC 2.3.1.16). Beta-ketothiolase enzymes are also required for the conversion of lactoyl-CoA and acetyl-CoA to 3-oxo-4-hydroxypentanoyl-CoA (FIG. 5A) and succinyl-CoA and acetyl-CoA to 3-oxoadipyl-CoA (FIG. 6A). Exemplary beta-ketothiolase enzymes are described below.

Beta-ketovaleryl-CoA thiolase catalyzes the formation of beta-ketovalerate from acetyl-CoA and propionyl-CoA. Zoogloea ramigera possesses two ketothiolases that can form beta-ketovaleryl-CoA from propionyl-CoA and acetyl-CoA and R. eutropha has a beta-oxidation ketothiolase that is also capable of catalyzing this transformation (Gruys et al., U.S. Pat. No. 5,958,745). The sequences of these genes or their translated proteins have not been reported, but several genes in R. eutropha, Z. ramigera, or other organisms can be identified based on sequence homology to bktB from R. eutropha.

| Protein | GenBank ID | GI Number | Organism |
|---|---|---|---|
| phaA | YP_725941.1 | 113867452 | Ralstonia eutropha |
| h16_A1713 | YP_726205.1 | 113867716 | Ralstonia eutropha |
| pcaF | YP_728366.1 | 116694155 | Ralstonia eutropha |
| h16_B1369 | YP_840888.1 | 116695312 | Ralstonia eutropha |
| h16_A0170 | YP_724690.1 | 113866201 | Ralstonia eutropha |
| h16_A0462 | YP_724980.1 | 113866491 | Ralstonia eutropha |
| h16_A1528 | YP_726028.1 | 113867539 | Ralstonia eutropha |
| h16_B0381 | YP_728545.1 | 116694334 | Ralstonia eutropha |
| h16_B0662 | YP_728824.1 | 116694613 | Ralstonia eutropha |
| h16_B0759 | YP_728921.1 | 116694710 | Ralstonia eutropha |
| h16_B0668 | YP_728830.1 | 116694619 | Ralstonia eutropha |
| h16_A1720 | YP_726212.1 | 113867723 | Ralstonia eutropha |
| h16_A1887 | YP_726356.1 | 113867867 | Ralstonia eutropha |
| phbA | P07097.4 | 135759 | Zoogloea ramigera |
| bktB | YP_002005382.1 | 194289475 | Cupriavidus taiwanensis |
| Rmet_1362 | YP_583514.1 | 94310304 | Ralstonia metallidurans |
| Bphy_0975 | YP_001857210.1 | 186475740 | Burkholderia phymatum |

Acetoacetyl-CoA thiolase converts two molecules of acetyl-CoA into acetoacetyl-CoA (EC 2.1.3.9). Exemplary acetoacetyl-CoA thiolase enzymes include the gene products of atoB from E. coli (Martin et al., Nat. Biotechnol. 21:796-802 (2003)), thlA and thlB from C. acetobutylicum (Hanai et al., Appl. Environ. Microbiol. 73:7814-7818 (2007); Winzer et al., J. Mol. Microbiol. Biotechnol. 2:531-541 (2000)), and ERG10 from S. cerevisiae (Hiser et al., J. Biol. Chem. 269:31383-31389 (1994)).

| Protein | GenBank ID | GI Number | Organism |
|---|---|---|---|
| atoB | NP_416728 | 16130161 | Escherichia coli |
| thlA | NP_349476.1 | 15896127 | Clostridium acetobutylicum |
| thlB | NP_149242.1 | 15004782 | Clostridium acetobutylicum |
| ERG10 | NP_015297 | 6325229 | Saccharomyces cerevisiae |

Beta-ketoadipyl-CoA thiolase (EC 2.3.1.174), also called 3-oxoadipyl-CoA thiolase, converts beta-ketoadipyl-CoA to succinyl-CoA and acetyl-CoA, and is a key enzyme of the beta-ketoadipate pathway for aromatic compound degradation. The enzyme is widespread in soil bacteria and fungi including Pseudomonas putida (Harwood et al., J. Bacteriol. 176-6479-6488 (1994)) and Acinetobacter calcoaceticus (Doten et al., J. Bacteriol. 169:3168-3174 (1987)). The P. putida enzyme is a homotetramer bearing 45% sequence homology to beta-ketothiolases involved in PHB synthesis in Ralstonia eutropha, fatty acid degradation by human mitochondria and butyrate production by Clostridium acetobutylicum (Harwood et al., supra). A beta-ketoadipyl-CoA thiolase in Pseudomonas knackmussii (formerly sp. B13) has also been characterized (Gobel et al., J. Bacteriol. 184:216-223 (2002); Kaschabek et al., supra).

| Protein | GenBank ID | GI Number | Organism |
|---|---|---|---|
| pcaF | NP_743536.1 | 506695 | Pseudomonas putida |
| pcaF | AAC37148.1 | 141777 | Acinetobacter calcoaceticus |
| catF | Q8VPF1.1 | 75404581 | Pseudomonas knackmussii |

### 3-Oxopent-4-Enoyl-CoA Hydrolase, Transferase or Synthase (FIG. 16, Step B)

### 3-Oxo-4-Hydroxypentanoyl-CoA Hydrolase, Transferase or Synthase (FIG. 17, Step B)

### 3,4-Dihydroxypentanoyl-CoA Hydrolase, Transferase or Synthase (FIG. 17, Step F)

### Oxoadipyl-CoA Hydrolase, Transferase or Synthase (FIG. 18, Step 6B)

Acyl-CoA hydrolase, transferase and synthase enzymes convert acyl-CoA moieties to their corresponding acids. Such an enzyme can be utilized to convert, for example, 3-oxopent-4-enoyl-CoA to 3-oxopent-4-enoyl-CoA, 3-oxo-4-hydroxypentanoyl-CoA to 3-oxo-4-hydroxypentanoate, 3,4-dihydroxypentanoyl-CoA to 3,4-dihydroxypentanoate or oxoadipyl-CoA to oxoadipate.

CoA hydrolase or thioesterase enzymes in the 3.1.2 family hydrolyze acyl-CoA molecules to their corresponding acids. Several CoA hydrolases with different substrate ranges are suitable for hydrolyzing 3-oxopent-4-enoyl-CoA, 3-oxo-4-hydroxypentanoyl-CoA, 3,4-dihydroxypentanoyl-CoA or oxoadipyl-CoA substrates to their corresponding acids. For example, the enzyme encoded by acot12 from Rattus norvegicus brain (Robinson et al., Biochem. Biophys. Res. Commun. 71:959-965 (1976)) can react with butyryl-CoA, hexanoyl-CoA and malonyl-CoA. The human dicarboxylic acid thioesterase, encoded by acot8, exhibits activity on glutaryl-CoA, adipyl-CoA, suberyl-CoA, sebacyl-CoA, and dodecanedioyl-CoA (Westin et al., J. Biol. Chem. 280:38125-38132 (2005)). The closest E. coli homolog to this enzyme, tesB, can also hydrolyze a range of CoA thiolesters (Naggert et al., J Biol Chem 266:11044-11050 (1991)). A similar enzyme has also been characterized in the rat liver (Deana R., Biochem Int 26:767-773 (1992)). Additional enzymes with hydrolase activity in E. coli include ybgC, paaI, and ybdB (Kuznetsova, et al., FEMS Microbiol Rev, 2005, 29(2):263-279; Song et al., J Biol Chem, 2006, 281(16):11028-38). Though its sequence has not been reported, the enzyme from the mitochondrion of the pea leaf has a broad substrate specificity, with demonstrated activity on acetyl-CoA, propionyl-CoA, butyryl-CoA, palmitoyl-CoA, oleoyl-CoA, succinyl-CoA, and crotonyl-CoA (Zeiher et al., Plant. Physiol. 94:20-27 (1990)) The acetyl-CoA hydrolase, ACH1, from S. cerevisiae represents another candidate hydrolase (Buu et al., J. Biol. Chem. 278:17203-17209 (2003)). Additional enzymes with aryl-CoA hydrolase activity include the palmitoyl-CoA hydrolase of Mycobacterium tuberculosis (Wang et al., Chem. Biol. 14:543-551 (2007)) and the acyl-CoA hydrolase of E. coli encoded by entH (Guo et al., Biochemistry 48:1712-1722 (2009)). Additional CoA hydrolase enzymes are described above.

| | GenBank | | |
|---|---|---|---|
| Gene name | Accession # | GI# | Organism |
| acot12 | NP_570103.1 | 18543355 | Rattus norvegicus |
| tesB | NP_414986 | 16128437 | Escherichia coli |
| acot8 | CAA15502 | 3191970 | Homo sapiens |
| acot8 | NP_570112 | 51036669 | Rattus norvegicus |
| tesA | NP_415027 | 16128478 | Escherichia coli |
| ybgC | NP_415264 | 16128711 | Escherichia coli |
| paaI | NP_415914 | 16129357 | Escherichia coli |
| ybdB | NP_415129 | 16128580 | Escherichia coli |
| ACH1 | NP_009538 | 6319456 | Saccharomyces cerevisiae |
| Rv0098 | NP_214612.1 | 15607240 | Mycobacterium tuberculosis |
| entH | AAC73698.1 | 1786813 | Escherichia coli |

CoA hydrolase enzymes active on 3-hydroxyacyl-CoA and 3-oxoacyl-CoA intermediates are well known in the art. 3-Hydroxyisobutyryl-CoA hydrolase is active on 3-hydroxyacyl-CoA substrates (Shimomura et al., J Biol Chem. 269:14248-14253 (1994)). Genes encoding this enzyme include hibch of Rattus norvegicus (Shimomura et al., Methods Enzymol. 324:229-240 (2000)) and Homo sapiens (Shimomura et al., supra). Similar gene candidates can also be identified by sequence homology, including hibch of Saccharomyces cerevisiae and BC_{—}2292 of Bacillus cereus. An exemplary 3-oxoacyl-CoA hydrolase is MKS2 of Solanum lycopersicum (Yu et al, Plant Physiol 154:67-77 (2010)). The native substrate of this enzyme is 3-oxo-myristoyl-CoA, which produces a C14 chain length product.

| Gene name | GenBank Accession # | GI# | Organism |
|---|---|---|---|
| fadM | NP_414977.1 | 16128428 | Escherichia coli |
| hibch | Q5XIE6.2 | 146324906 | Rattus norvegicus |
| hibch | Q6NVY1.2 | 146324905 | Homo sapiens |
| hibch | P28817.2 | 2506374 | Saccharomyces cerevisiae |
| BC_2292 | AP09256 | 29895975 | Bacillus cereus |
| MKS2 | ACG69783.1 | 196122243 | Solanum lycopersicum |

CoA transferases catalyze the reversible transfer of a CoA moiety from one molecule to another. Several transformations require a CoA transferase to acyl-CoA substrates to their corresponding acid derivatives. CoA transferase enzymes are known in the art and described below.

The gene products of cat 1, cat2, and cat3 of Clostridium kluyveri have been shown to exhibit succinyl-CoA, 4-hydroxybutyryl-CoA, and butyryl-CoA transferase activity, respectively (Seedorf et al., Proc. Natl. Acad. Sci U.S.A 105:2128-2133 (2008); Sohling et al., J Bacteriol. 178:871-880 (1996)). Similar CoA transferase activities are also present in Trichomonas vaginalis, Trypanosoma brucei, Clostridium aminobutyricum and Porphyromonas gingivalis (Riviere et al., J. Biol. Chem. 279:45337-45346 (2004); van Grinsven et al., J. Biol. Chem. 283:1411-1418 (2008)).

| | | GI | |
|---|---|---|---|
| Protein | GenBank ID | Number | Organism |
| cat1 | P38946.1 | 729048 | Clostridium kluyveri |
| cat2 | P38942.2 | 172046066 | Clostridium kluyveri |
| cat3 | EDK35586.1 | 146349050 | Clostridium kluyveri |
| TVAG_395550 | XP_001330176 | 123975034 | Trichomonas vaginalis G3 |
| Tb11.02.0290 | XP_828352 | 71754875 | Trypanosoma brucei |
| cat2 | CAB60036.1 | 6249316 | Clostridium aminobutyricum |
| cat2 | NP_906037.1 | 34541558 | Porphyromonas gingivalis |
| | | | W83 |

A fatty acyl-CoA transferase that utilizes acetyl-CoA as the CoA donor is acetoacetyl-CoA transferase, encoded by the E. coli atoA (alpha subunit) and atoD (beta subunit) genes (Korolev et al., Acta Crystallogr. D. Biol. Crystallogr. 58:2116-2121 (2002); Vanderwinkel et al., 33:902-908 (1968)). This enzyme has a broad substrate range on substrates of chain length C3-C6 (Sramek et al., Arch Biochem Biophys 171:14-26 (1975)) and has been shown to transfer the CoA moiety to acetate from a variety of branched and linear 3-oxo and acyl-CoA substrates, including isobutyrate (Matthies et al., Appl Environ. Microbiol 58:1435-1439 (1992)), valerate (Vanderwinkel et al., Biochem. Biophys. Res. Commun. 33:902-908 (1968)) and butanoate (Vanderwinkel et al., Biochem. Biophys. Res. Commun. 33:902-908 (1968)). This enzyme is induced at the transcriptional level by acetoacetate, so modification of regulatory control may be necessary for engineering this enzyme into a pathway (Pauli et al., Eur. J Biochem. 29:553-562 (1972)). Similar enzymes exist in Corynebacterium glutamicum ATCC 13032 (Duncan et al., 68:5186-5190 (2002)), Clostridium acetobutylicum (Cary et al., Appl Environ Microbiol 56:1576-1583 (1990); Wiesenborn et al., Appl Environ Microbiol 55:323-329 (1989)), and Clostridium saccharoperbutylacetonicum (Kosaka et al., Biosci. Biotechnol Biochem. 71:58-68 (2007)).

| Gene | GI # | Accession No. | Organism |
|---|---|---|---|
| atoA | 2492994 | P76459.1 | Escherichia coli |
| atoD | 2492990 | P76458.1 | Escherichia coli |
| actA | 62391407 | YP_226809.1 | Corynebacterium glutamicum |
| cg0592 | 62389399 | YP_224801.1 | Corynebacterium glutamicum |
| ctfA | 15004866 | NP_149326.1 | Clostridium acetobutylicum |
| ctfB | 15004867 | NP_149327.1 | Clostridium acetobutylicum |
| ctfA | 31075384 | AAP42564.1 | Clostridium |
| | | | saccharoperbutylacetonicum |
| ctfB | 31075385 | AAP42565.1 | Clostridium |
| | | | saccharoperbutylacetonicum |

Beta-ketoadipyl-CoA transferase, also known as succinyl-CoA:3:oxoacid-CoA transferase, is active on 3-oxoacyl-CoA substrates. This enzyme is encoded by pcaI and pcaJ in Pseudomonas putida (Kaschabek et al., J Bacteriol. 184:207-215 (2002)). Similar enzymes are found in Acinetobacter sp. ADP1 (Kowalchuk et al., Gene 146:23-30 (1994)), Streptomyces coelicolor and Pseudomonas knaclanussii (formerly sp. B13) (Gobel et al., J Bacteriol. 184:216-223 (2002); Kaschabek et al., J Bacteriol. 184:207-215 (2002)). Additional exemplary succinyl-CoA:3:oxoacid-CoA transferases have been characterized in Helicobacter pylori (Corthesy-Theulaz et al., J Biol. Chem. 272:25659-25667 (1997)), Bacillus subtilis (Stols et al., Protein Expr. Purif 53:396-403 (2007)) and Homo sapiens (Fukao, T., et al., Genomics 68:144-151 (2000); Tanaka, H., et al., Mol Hum Reprod 8:16-23 (2002)). Genbank information related to these genes is summarized below.

| Gene | GI # | Accession No. | Organism |
|---|---|---|---|
| pcaI | 24985644 | AAN69545.1 | Pseudomonas putida |
| pcaJ | 26990657 | NP_746082.1 | Pseudomonas putida |
| pcaI | 50084858 | YP_046368.1 | Acinetobacter sp. ADP1 |
| pcaJ | 141776 | AAC37147.1 | Acinetobacter sp. ADP1 |
| pcaI | 21224997 | NP_630776.1 | Streptomyces coelicolor |
| pcaJ | 21224996 | NP_630775.1 | Streptomyces coelicolor |
| catI | 75404583 | Q8VPF3 | Pseudomonas knackmussii |
| catJ | 75404582 | Q8VPF2 | Pseudomonas knackmussii |
| HPAG1_0676 | 108563101 | YP_627417 | Helicobacter pylori |
| HPAG1_0677 | 108563102 | YP_627418 | Helicobacter pylori |
| ScoA | 16080950 | NP_391778 | Bacillus subtilis |
| ScoB | 16080949 | NP_391777 | Bacillus subtilis |
| OXCT1 | NP_000427 | 4557817 | Homo sapiens |
| OXCT2 | NP_071403 | 11545841 | Homo sapiens |

The conversion of acyl-CoA substrates to their acid products can be catalyzed by a CoA acid-thiol ligase or CoA synthetase in the 6.2.1 family of enzymes. CoA synthases that convert ATP to ADP (ADP-forming) are reversible and react in the direction of acid formation, whereas AMP forming enzymes only catalyze the activation of an acid to an acyl-CoA. For fatty acid formation, deletion or attenuation of AMP forming enzymes will reduce backflux. ADP-forming acetyl-CoA synthetase (ACD, EC 6.2.1.13) is an enzyme that couples the conversion of acyl-CoA esters to their corresponding acids with the concomitant synthesis of ATP. ACD I from Archaeoglobus fulgidus, encoded by AF1211, was shown to operate on a variety of linear and branched-chain substrates including isobutyrate, isopentanoate, and fumarate (Musfeldt et al., J Bacteriol. 184:636-644 (2002)). A second reversible ACD in Archaeoglobus fulgidus, encoded by AF 1983, was also shown to have a broad substrate range (Musfeldt and Schonheit, J Bacteriol. 184:636-644 (2002)). The enzyme from Haloarcula marismortui (annotated as a succinyl-CoA synthetase) accepts propionate, butyrate, and branched-chain acids (isovalerate and isobutyrate) as substrates, and was shown to operate in the forward and reverse directions (Brasen et al., Arch Microbiol 182:277-287 (2004)). The ACD encoded by PAE3250 from hyperthermophilic crenarchaeon Pyrobaculum aerophilum showed the broadest substrate range of all characterized ACDs, reacting with acetyl-CoA, isobutyryl-CoA (preferred substrate) and phenylacetyl-CoA (Brasen et al, supra). Directed evolution or engineering can be used to modify this enzyme to operate at the physiological temperature of the host organism. The enzymes from A. fulgidus, H. marismortui and P. aerophilum have all been cloned, functionally expressed, and characterized in E. coli (Brasen and Schonheit, supra; Musfeldt and Schonheit, J Bacteriol. 184:636-644 (2002)). An additional candidate is succinyl-CoA synthetase, encoded by sucCD of E. coli and LSC1 and LSC2 genes of Saccharomyces cerevisiae. These enzymes catalyze the formation of succinyl-CoA from succinate with the concomitant consumption of one ATP in a reaction which is reversible in vivo (Buck et al., Biochemistry 24:6245-6252 (1985)). The acyl CoA ligase from Pseudomonas putida has been demonstrated to work on several aliphatic substrates including acetic, propionic, butyric, valeric, hexanoic, heptanoic, and octanoic acids and on aromatic compounds such as phenylacetic and phenoxyacetic acids (Fernandez-Valverde et al., Appl. Environ. Microbiol. 59:1149-1154 (1993)). A related enzyme, malonyl CoA synthetase (6.3.4.9) from Rhizobium leguminosarum could convert several diacids, namely, ethyl-, propyl-, allyl-, isopropyl-, dimethyl-, cyclopropyl-, cyclopropylmethylene-, cyclobutyl-, and benzyl-malonate into their corresponding monothioesters (Pohl et al., J. Am. Chem. Soc. 123:5822-5823 (2001)).

| Protein | GenBank ID | GI Number | Organism |
|---|---|---|---|
| AF1211 | NP_070039.1 | 11498810 | Archaeoglobus fulgidus |
| AF1983 | NP_070807.1 | 11499565 | Archaeoglobus fulgidus |
| scs | YP_135572.1 | 55377722 | Haloarcula marismortui |
| PAE3250 | NP_560604.1 | 18313937 | Pyrobaculum aerophilum str. IM2 |
| sucC | NP_415256.1 | 16128703 | Escherichia coli |
| sucD | AAC73823.1 | 1786949 | Escherichia coli |
| LSC1 | NP_014785 | 6324716 | Saccharomyces cerevisiae |
| LSC2 | NP_011760 | 6321683 | Saccharomyces cerevisiae |
| paaF | AAC24333.2 | 22711873 | Pseudomonas putida |
| matB | AAC83455.1 | 3982573 | Rhizobium leguminosarum |

### 3-Oxopent-4-Enoate Decarboxylase, 3-Oxoadipate Decarboxylase (FIG. 16, Step C, FIG. 18, Step C)

Decarboxylase enzymes suitable for decarboxylating 3-ketoacids such as 3-oxopent-4-enoate (FIG. 4C) and 3-oxoadipate (FIG. 6C) include acetoacetate decarboxylase (EC 4.1.1.4), arylmalonate decarboxylase and 3-oxoacid decarboxylase (EC 4.1.1.-). The 3-oxoacid decarboxylase of Lycopersicon hirsutum f. glabratum, encoded by MKS1, decarboxylates a range of 3-ketoacids to form methylketones (Yu et al, Plant Physiol 154: 67-77 (2010)). This enzyme has been functionally expressed in E. coli, where it was active on the substrate 3-ketomyristic acid. Homologous 3-oxoacid decarboxylase genes in Solanum lycopersicum are listed in the table below. Acetoacetate decarboxylase decarboxylates acetoacetate to acetone. The enzyme from Clostridium acetobutylicum, encoded by adc, has a broad substrate specificity and has been shown to decarboxylate 2-methyl-3-oxobutyrate, 3-oxohexanoate, phenyl acetoacetate and 2-ketocyclohexane-1-carboxylate (Rozzel et al., J. Am. Chem. Soc. 106:4937-4941 (1984); Benner and Rozzell, J. Am. Chem. Soc. 103:993-994 (1981); Autor et al., J Biol. Chem. 245:5214-5222 (1970)). A similar acetoacetate decarboxylase has also been characterized in Clostridium beijerinckii (Ravagnani et al., Mol. Microbiol 37:1172-1185 (2000)). An acetoacetate decarboxylase enzyme from Paenibacillus polymyxa, characterized in cell-free extracts, also has a broad substrate specificity for 3-keto acids and can decarboxylate 3-oxopentanoate (Matiasek et al., Curr. Microbiol 42:276-281 (2001)). The P. polymyxa genome encodes several acetoacetate decarboxylase enzymes, listed in the table below (Niu et al, J Bacteriol 193: 5862-3 (2011)). Another adc is found in Clostridium saccharoperbutylacetonicum (Kosaka, et al., Biosci. Biotechnol Biochem. 71:58-68 (2007)). Additional gene candidates in other organisms, including Clostridium botulinum and Bacillus amyloliquefaciens, can be identified by sequence homology. Arylmalonate decarboxylase (AMDase) catalyzes the decarboxylation of malonate and a range of alpha-substituted derivatives (phenylmalonic acid, 2-methyl-2-phenylmalonic acid, 2-methyl-2-napthylmalonic acid, 2-thienylmalonic acid). AMDase is unusual in that it does not require biotin or other cofactors for activity. Exemplary AMDase enzymes are found in US Patent Application 2010/0311037. A codon optimized variant of the B. bronchiseptica enzyme was heterologously expressed in E. coli and crystallized. Acetolactate decarboxylase enzyme candidates, described above (FIG. 2B) are also applicable here.

| Protein | GenBank ID | GI Number | Organism |
|---|---|---|---|
| MKS1 | ADK38535.1 | 300836815 | Lycopersicon hirsutum f. glabratum |
| MKS1a | ADK38537.1 | 300836819 | Solanum lycopersicum |
| MKS1b | ADK38538.1 | 300836821 | Solanum lycopersicum |
| MKS1c | ADK38543.1 | 300836832 | Solanum lycopersicum |
| MKS1d | ADK38539.1 | 300836824 | Solanum lycopersicum |
| MKS1e | ADK38540.1 | 300836826 | Solanum lycopersicum |
| adc | NP_149328.1 | 15004868 | Clostridium acetobutylicum |
| adc | AAP42566.1 | 31075386 | Clostridium saccharoperbutylacetonicum |
| adc | YP_001310906.1 | 150018652 | Clostridium beijerinckii |
| Adc3 | YP_005960063.1 | 386041109 | Paenibacillus polymyxa |
| Adc1 | YP_005958789.1 | 386039835 | Paenibacillus polymyxa |
| CLL_A2135 | YP_001886324.1 | 187933144 | Clostridium botulinum |
| RBAM_030030 | YP_001422565.1 | 154687404 | Bacillus amyloliquefaciens |
| S54007.1:545 . . . 1267 | AAC60426.1 | 298239 | Bordetella bronchiseptica KU1201 |

Alternatively, decarboxylation of 3-ketoacids can occur spontaneously in the absence of a decarboxylase enzyme. 3-Ketoacids are known to be inherently unstable and prone to decarboxylation (Kornberg et al, Fed Proc 6:268 (1947)). In one recent study, high yields of methyl ketones were formed from 3-oxoacids in reaction mixtures lacking decarboxylase enzymes (Goh et al, AEM 78: 70-80 (2012)).

### 3-Buten-2-One Reductase (FIG. 16, Step D)

### 4-Oxopentanoate Reductase (FIG. 18, Step D)

### 3-Oxo-4-Hydroxypentanoate Reductase (FIG. 17, Step C)

Reduction of 3-buten-2-one to 3-buten-2-ol, 4-oxopentanoate to 4-hydroxypentanoate, or 3-oxo-4-hydroxypentanoate to 3,4-dihydroxypentanoate, is catalyzed by secondary alcohol dehydrogenase or ketone reductase enzymes. Secondary alcohol dehydrogenase enzymes of C. beijerinckii (Ismaiel et al., J. Bacteriol. 175:5097-5105 (1993)) and T. brockii (Lamed et al., Biochem. J. 195:183-190 (1981); Peretz et al., Biochemistry. 28:6549-6555 (1989)) convert acetone to isopropanol. Methyl ethyl ketone reductase catalyzes the reduction of MEK to 2-butanol. Exemplary MEK reductase enzymes can be found in Rhodococcus ruber (Kosjek et al., Biotechnol Bioeng. 86:55-62 (2004)) and Pyrococcus furiosus (van der Oost et al., Eur. J. Biochem. 268:3062-3068 (2001)). The cloning of the bdhA gene from Rhizobium (Sinorhizobium) meliloti into E. coli conferred the ability to utilize 3-hydroxybutyrate as a carbon source (Aneja and Charles, J Bacteriol. 181(3):849-857 (1999)). Additional gene candidates can be found in Pseudomonas fragi (Ito et al., J. Mol. Biol. 355(4) 722-733 (2006)) and Ralstonia pickettii (Takanashi et al., Antonie van Leeuwenoek, 95(3):249-262 (2009)). Recombinant 3-ketoacid reductase enzymes with broad substrate range and high activity have been characterized in US Application 2011/0201072, and are incorporated by reference herein. The mitochondrial 3-hydroxybutyrate dehydrogenase (bdh) from the human heart has been cloned and characterized (Marks et al., J. Biol. Chem. 267:15459-15463 (1992)). Yet another secondary ADH, sadH of Candida parapsilosis, demonstrated activity on 3-oxobutanol (Matsuyama et al. J Mol Cat B Enz, 11:513-521 (2001)). Enzyme candidates for converting acrolein to 2,3-butanediol (Step 2C) and 2-butanone to 2-butanol (Step E) are also applicable here.

| | GenBank | | |
|---|---|---|---|
| Gene | Accession No. | GI No. | Organism |
| adh | AAA23199.2 | 60592974 | Clostridium beijerinckii NRRL |
| | | | B593 |
| adh | P14941.1 | 113443 | Thermoanaerobacter brockii HTD4 |
| sadh | CAD36475 | 21615553 | Rhodococcus ruber |
| adhA | AAC25556 | 3288810 | Pyrococcus furiosus |
| PRK13394 | BAD86668.1 | 57506672 | Pseudomonas fragi |
| Bdh1 | BAE72684.1 | 84570594 | Ralstonia pickettii |
| Bdh2 | BAE72685.1 | 84570596 | Ralstonia pickettii |
| Bdh3 | BAF91602.1 | 158937170 | Ralstonia pickettii |
| bdh | AAA58352.1 | 177198 | Homo sapiens |
| sadh | BAA24528.1 | 2815409 | Candida parapsilosis |

Allyl alcohol dehydrogenase enzymes are suitable for reducing 3-buten-2-one to 3-buten-2-ol. An exemplary allyl alcohol dehydrogenase is the NtRed-1 enzyme from Nicotiana tabacum (Matsushima et al, Bioorg Chem 36: 23-8 (2008)). A similar enzyme has been characterized in Pseudomonas putida MB1 but the enzyme has not been associated with a gene to date (Malone et al, AEM 65: 2622-30 (1999)). Yet another allyl alcohol dehydrogenase is the geraniol dehydrogenase enzymes of Castellaniella defragrans, Carpoglyphus lactis and Ocimum basilicum (Lueddeke et al, AEM 78:2128-36 (2012)).

| | GenBank | | |
|---|---|---|---|
| Gene | Accession No. | GI No. | Organism |
| NT-RED1 | BAA89423 | 6692816 | Nicotiana tabacum |
| geoA | CCF55024.1 | 372099287 | Castellaniella defragrans |
| GEDH1 | Q2KNL6.1 | 122200955 | Ocimum basilicum |
| GEDH | BAG32342.1 | 188219500 | Carpoglyphus lactis |

### 3-Oxo-4-Hydroxypentanoyl-CoA Reductase (FIG. 17, Step E)

Reduction of 3-oxo-4-hydroxypentanoyl-CoA to 3,4-dihydroxypentanoyl-CoA (FIG. 5E) is catalyzed by a 3-hydroxyacyl-CoA dehydrogenase (also called 3-oxoacyl-CoA reductase). 3-Hydroxyacyl-CoA dehydrogenase enzymes are often involved in fatty acid beta-oxidation and aromatic degradation pathways. For example, subunits of two fatty acid oxidation complexes in E. coli, encoded by fadB and fadJ, function as 3-hydroxyacyl-CoA dehydrogenases (Binstock et al., Methods Enzymol. 71 Pt C:403-411 (1981)). Knocking out a negative regulator encoded by fadR can be utilized to activate the fadB gene product (Sato et al., J Biosci. Bioeng 103:38-44 (2007)). Another 3-hydroxyacyl-CoA dehydrogenase from E. coli is paaH (Ismail et al., European Journal of Biochemistry 270:3047-3054 (2003)). Additional 3-oxoacyl-CoA enzymes include the gene products of phaC in Pseudomonas putida (Olivera et al., Proc. Natl. Acad. Sci U.S.A 95:6419-6424 (1998)) and paaC in Pseudomonas fluorescens (Di et al., 188:117-125 (2007)). These enzymes catalyze the reversible oxidation of 3-hydroxyadipyl-CoA to 3-oxoadipyl-CoA during the catabolism of phenylacetate or styrene. Other suitable enzyme candidates include AAO72312.1 from E. gracilis (Winkler et al., Plant Physiology 131:753-762 (2003)) and paaC from Pseudomonas putida (Olivera et al., PNAS USA 95:6419-6424 (1998)). Enzymes catalyzing the reduction of acetoacetyl-CoA to 3-hydroxybutyryl-CoA include hbd of Clostridium acetobutylicum (Youngleson et al., J Bacteriol. 171:6800-6807 (1989)), phbB from Zoogloea ramigera (Ploux et al., Eur. J Biochem. 174:177-182 (1988)), phaB from Rhodobacter sphaeroides (Alber et al., Mol. Microbiol 61:297-309 (2006)) and paaH1 of Ralstonia eutropha (Machado et al, Met Eng, In Press (2012)). The Z. ramigera enzyme is NADPH-dependent and also accepts 3-oxopropionyl-CoA as a substrate (Ploux et al., Eur. J Biochem. 174:177-182 (1988)). Additional genes include phaB in Paracoccus denitrificans, Hbd1 (C-terminal domain) and Hbd2 (N-terminal domain) in Clostridium kluyveri (Hillmer and Gottschalk, Biochim. Biophys. Acta 3334:12-23 (1974)) and HSD17B10 in Bos taurus (Wakil et al., J Biol. Chem. 207:631-638 (1954)). The enzyme from Paracoccus denitrificans has been functionally expressed and characterized in E. coli (Yabutani et al., FEMS Microbiol Lett. 133:85-90 (1995)). A number of similar enzymes have been found in other species of Clostridia and in Metallosphaera sedula (Berg et al., Science. 318:1782-1786 (2007)). The enzyme from Candida tropicalis is a component of the peroxisomal fatty acid beta-oxidation multifunctional enzyme type 2 (MFE-2). The dehydrogenase B domain of this protein is catalytically active on acetoacetyl-CoA. The domain has been functionally expressed in E. coli, a crystal structure is available, and the catalytic mechanism is well-understood (Ylianttila et al., Biochem Biophys Res Commun 324:25-30 (2004); Ylianttila et al., J Mol Biol 358:1286-1295 (2006)). 3-Hydroxyacyl-CoA dehydrogenases that accept longer acyl-CoA substrates (eg. EC 1.1.1.35) are typically involved in beta-oxidation. An example is HSD17B10 in Bos taurus (Wakil et al., J Biol. Chem. 207:631-638 (1954)). The pig liver enzyme is preferentially active on short and medium chain acyl-CoA substrates whereas the heart enzyme is less selective (He et al, Biochim Biophys Acta 1392:119-26 (1998)). The S. cerevisiae enzyme FOX2 is active in beta-degradation pathways and also has enoyl-CoA hydratase activity (Hiltunen et al, J Biol Chem 267: 6646-6653 (1992)).

| Protein | GENBANK ID | GI NUMBER | ORGANISM |
|---|---|---|---|
| fadB | P21177.2 | 119811 | Escherichia coli |
| fadJ | P77399.1 | 3334437 | Escherichia coli |
| paaH | NP_415913.1 | 16129356 | Escherichia coli |
| Hbd2 | EDK34807.1 | 146348271 | Clostridium kluyveri |
| Hbd1 | EDK32512.1 | 146345976 | Clostridium kluyveri |
| phaC | NP_745425.1 | 26990000 | Pseudomonas putida |
| paaC | ABF82235.1 | 106636095 | Pseudomonas fluorescens |
| HSD17B10 | O02691.3 | 3183024 | Bos taurus |
| phbB | P23238.1 | 130017 | Zoogloea ramigera |
| phaB | YP_353825.1 | 77464321 | Rhodobacter sphaeroides |
| paaH1 | CAJ91433.1 | 113525088 | Ralstonia eutropha |
| phaB | BAA08358 | 675524 | Paracoccus denitrificans |
| Hbd | NP_349314.1 | 15895965 | Clostridium acetobutylicum |
| Hbd | AAM14586.1 | 20162442 | Clostridium beijerinckii |
| Msed_1423 | YP_001191505 | 146304189 | Metallosphaera sedula |
| Msed_0399 | YP_001190500 | 146303184 | Metallosphaera sedula |
| Msed_0389 | YP_001190490 | 146303174 | Metallosphaera sedula |
| Msed_1993 | YP_001192057 | 146304741 | Metallosphaera sedula |
| Fox2 | Q02207 | 399508 | Candida tropicalis |
| HSD17B10 | O02691.3 | 3183024 | Bos taurus |
| HADH | NP_999496.1 | 47523722 | Bos taurus |
| 3HCDH | AAO72312.1 | 29293591 | Euglena gracilis |
| FOX2 | NP_012934.1 | 6322861 | Saccharomyces cerevisiae |

### Example X

### Pathways for Converting Lactoyl-CoA to 3-Buten-2-Ol and/or Butadiene

This example describes pathways for converting lactoyl-CoA to 3-buten-2-ol, and further to butadiene. The conversion of lactoyl-CoA to 3-buten-2-ol is accomplished in four enzymatic steps. Lactoyl-CoA and acetyl-CoA are first condensed to 3-oxo-4-hydroxypentanoyl-CoA by 3-oxo-4-hydroxypentanoyl-CoA thiolase, a beta-ketothiolase (Step 17A). In one pathway, the 3-oxo-4-hydroxypentanoyl-CoA product is converted to its corresponding acid by a CoA hydrolase, transferase or synthetase (Step 17B). Reduction of the 3-oxo ketone by an alcohol dehydrogenase yields 3,4-dihydroxypentanoate (Step 17C). Alternately, 3,4-dihydroxypentanoate intermediate is formed from 3-oxo-4-hydroxypentanoyl-CoA by a 3-oxo-4-hydroxypentanoyl-CoA reductase and a 3,4-dihydroxypentanoyl-CoA transferase, synthetase or hydrolase (Steps E and F, respectively). Decarboxylation of 3,4-dihydroxypentanoate yields 3-buten-2-ol (Step 17D). 3-Buten-2-ol is further converted to butadiene via chemical dehydration or by a dehydratase enzyme (Step 17G). In an alternate pathway, 3,4-dihydroxypentanoate is dehydrated to 4-oxopentanoate by a diol dehydratase (Step 17H). 4-Oxopentanoate is reduced to 4-hydroxypentanoate, and then decarboxylated to 3-buten-2-ol by an alkene-forming decarboxylase (Steps 17I-17J).

Enzymes and gene candidates for catalyzing but-3-en-2-ol and butadiene pathway reactions are described in further detail below. Enzymes for catalyzing steps A, B, C, E, F, G and H are described above.

### 3,4-Dihydroxypentanoate Decarboxylase (FIG. 17, Step D)

Olefin-forming decarboxylase enzymes suitable for converting 3,4-dihydroxypentanoate to 3-buten-2-ol include mevalonate diphosphate decarboxylase (MDD, EC 4.1.1.33) and similar enzymes. MDD participates in the mevalonate pathway for isoprenoid biosynthesis, where it catalyzes the ATP-dependent decarboxylation of mevalonate diphosphate to isopentenyl diphosphate. The MDD enzyme of S. cerevisiae was heterologously expressed in E. coli, where it was shown to catalyze the decarboxylation of 3-hydroxyacids to their corresponding alkenes (WO 2010/001078; Gogerty and Bobik, Appl. Environ. Microbiol., p. 8004-8010, Vol. 76, No. 24 (2010)). Products formed by this enzyme include isobutylene, propylene and ethylene. Two evolved variants of the S. cerevisiae MDD, ScMDD1 (I145F) and ScMDD2 (R74H), achieved 19-fold and 38-fold increases in isobutylene-forming activity compared to the wild-type enzyme (WO 2010/001078). Other exemplary MDD genes are MVD in Homo sapiens and MDD in Staphylococcus aureus and Trypsonoma brucei (Toth et al., J Biol. Chem. 271:7895-7898 (1996); Byres et al., J Mol. Biol. 371:540-553 (2007)).

| Protein | GenBank ID | GI Number | Organism |
|---|---|---|---|
| MDD | NP_014441.1 | 6324371 | Saccharomyces cerevisiae |
| MVD | NP_002452.1 | 4505289 | Homo sapiens |
| MDD | ABQ48418.1 | 147740120 | Staphylococcus aureus |
| MDD | EAN78728.1 | 70833224 | Trypsonoma brucei |

### 4-Hydroxypentanoate Decarboxylase (FIG. 17, Step J and FIG. 18, Step E)

An olefin-forming decarboxylase enzyme catalyzes the conversion of 4-hydroxypentanoate to 3-buten-2-ol. An exemplary terminal olefin-forming fatty acid decarboxylase is encoded by the oleT gene product of Jeotgalicoccus sp. ATCC8456 (Rude et al, AEM 77(5):1718-27 (2011)). This enzyme is a member of the cytochrome P450 family of enzymes and is similar to P450s that catalyze fatty acid hydroxylation. OleT and homologs are listed in the table below. Additional olefin-forming fatty acid decarboxylase enzymes are described in US 2011/0196180 and WO/2013028792.

| Protein | GenBank ID | GI Number | Organism |
|---|---|---|---|
| oleT | ADW41779.1 | 320526718 | Jeotgalicoccus sp. ATCC8456 |
| MCCL_0804 | BAH17511.1 | 222120176 | Macrococcus caseolyticus |
| SPSE _1582 | ADX76840.1 | 323464687 | Staphylococcus pseudintermedius |
| faaH | ADC49546.1 | 288545663 | Bacillus pseudofirmus |
| cypC2 | EGQ19322.1 | 339614630 | Sporosarcina newyorkensis |
| cypC | BAK15372.1 | 32743900 | Solibacillus silvestris |
| Bcoam_010100017440 | ZP_03227611.1 | 205374818 | Bacillus coahuilensis |
| SYNPCC7002_A2265 | YP_001735499.1 | 170078861 | Synechococcus sp. PCC 7002 |
| Cyan7822_1848 | YP_003887108.1 | 307151724 | Cyanothece sp. PCC 7822 |
| PCC7424_1874 | YP_002377175 | 218438846 | Cyanothece sp. PCC 7424 |
| LYNGBM3L 11290 | ZP_08425909.1 | 332705833 | Lyngbya majuscule 3L |
| LYNGBM3L_74520 | ZP_08432358.1 | 332712432 | Lyngbya majuscule 3L |
| Hoch_0800 | YP_003265309 | 262194100 | Haliangium ochraceum DSM 14365 |

### 3,4-Dihydroxypentanoate Dehydratase (FIG. 17, Step H)

A diol dehydratase enzyme with activity on 3,4-dihydroxypentanoate is required to form 4-oxopentanoate in FIG. 5H. Exemplary diol dehydratase enzymes described above for the dehydration of 2,3-butanediol to 2-butanol are also applicable here. Additional diol dehydratase enzymes are listed in the table below.

| Enzyme | |
|---|---|
| Commission No. | Enzyme Name |
| 4.2.1.5 | arabinonate dehydratase |
| 4.2.1.6 | galactonate dehydratase |
| 4.2.1.7 | altronate dehydratase |
| 4.2.1.8 | mannonate dehydratase |
| 4.2.1.9 | dihydroxy-acid dehydratase |
| 4.2.1.12 | phosphogluconate dehydratase |
| 4.2.1.25 | L-arabinonate dehydratase |
| 4.2.1.28 | propanediol dehydratase |
| 4.2.1.30 | glycerol dehydratase |
| 4.2.1.32 | L(+)-tartrate dehydratase |
| 4.2.1.39 | gluconate dehydratase |
| 4.2.1.40 | glucarate dehydratase |
| 4.2.1.41 | 5-dehydro-4-deoxyglucarate dehydratase |
| 4.2.1.42 | galactarate dehydratase |
| 4.2.1.43 | 2-dehydro-3-deoxy-L-arabinonate dehydratase |
| 4.2.1.44 | myo-inosose-2 dehydratase |
| 4.2.1.45 | CDP-glucose 4,6-dehydratase |
| 4.2.1.46 | dTDP-glucose 4,6-dehydratase |
| 4.2.1.47 | GDP-mannose 4,6-dehydratase |
| 4.2.1.76 | UDP-glucose 4,6-dehydratase |
| 4.2.1.81 | D(−)-tartrate dehydratase |
| 4.2.1.82 | xylonate dehydratase |
| 4.2.1.90 | L-rhamnonate dehydratase |
| 4.2.1.109 | methylthioribulose 1-phosphate dehydratase |

Diol dehydratase enzymes include dihydroxy-acid dehydratase (EC 4.2.1.9), propanediol dehydratase (EC 4.2.1.28), glycerol dehydratase (EC 4.2.1.30) and myo-inositose dehydratase (EC 4.2.1.44).

Adenosylcobalamin-dependent diol dehydratases contain alpha, beta and gamma subunits, which are all required for enzyme function. Exemplary propanediol dehydratase candidates are found in Klebsiella pneumoniae (Toraya et al., Biochem. Biophys. Res. Commun. 69:475-480 (1976); Tobimatsu et al., Biosci. Biotechnol Biochem. 62:1774-1777 (1998)), Salmonella typhimurium (Bobik et al., J Bacteriol. 179:6633-6639 (1997)), Klebsiella oxytoca (Tobimatsu et al., J Biol. Chem. 270:7142-7148 (1995)) and Lactobacillus collinoides (Sauvageot et al., FEMS Microbiol Lett. 209:69-74 (2002)). Methods for isolating diol dehydratase gene candidates in other organisms are well known in the art (e.g. U.S. Pat. No. 5,686,276).

| Protein | GenBank ID | GI Number | Organism |
|---|---|---|---|
| pddC | AAC98386.1 | 4063704 | Klebsiella pneumoniae |
| pddB | AAC98385.1 | 4063703 | Klebsiella pneumoniae |
| pddA | AAC98384.1 | 4063702 | Klebsiella pneumoniae |
| pduC | AAB84102.1 | 2587029 | Salmonella typhimurium |
| pduD | AAB84103.1 | 2587030 | Salmonella typhimurium |
| pduE | AAB84104.1 | 2587031 | Salmonella typhimurium |
| pddA | BAA08099.1 | 868006 | Klebsiella oxytoca |
| pddB | BAA08100.1 | 868007 | Klebsiella oxytoca |
| pddC | BAA08101.1 | 868008 | Klebsiella oxytoca |
| pduC | CAC82541.1 | 18857678 | Lactobacillus collinoides |
| pduD | CAC82542.1 | 18857679 | Lactobacillus collinoides |
| pduE | CAD01091.1 | 18857680 | Lactobacillus collinoides |

Enzymes in the glycerol dehydratase family (EC 4.2.1.30) are also diol dehydratases. Exemplary gene candidates are encoded by gldABC and dhaB123 in Klebsiella pneumoniae (World Patent WO 2008/137403) and (Toraya et al., Biochem. Biophys. Res. Commun. 69:475-480 (1976)), dhaBCE in Clostridium pasteuranum (Macis et al., FEMS Microbiol Lett. 164:21-28 (1998)) and dhaBCE in Citrobacter freundii (Seyfried et al., J Bacteriol. 178:5793-5796 (1996)). Variants of the B12-dependent diol dehydratase from K. pneumoniae with 80- to 336-fold enhanced activity were recently engineered by introducing mutations in two residues of the beta subunit (Qi et al., J. Biotechnol. 144:43-50 (2009)). Diol dehydratase enzymes with reduced inactivation kinetics were developed by DuPont using error-prone PCR (WO 2004/056963).

| Protein | GenBank ID | GI Number | Organism |
|---|---|---|---|
| gldA | AAB96343.1 | 1778022 | Klebsiella pneumonia |
| gldB | AAB96344.1 | 1778023 | Klebsiella pneumonia |
| gldC | AAB96345.1 | 1778024 | Klebsiella pneumoniae |
| dhaB1 | ABR78884.1 | 150956854 | Klebsiella pneumoniae |
| dhaB2 | ABR78883.1 | 150956853 | Klebsiella pneumoniae |
| dhaB3 | ABR78882.1 | 150956852 | Klebsiella pneumoniae |
| dhaB | AAC27922.1 | 3360389 | Clostridium pasteuranum |
| dhaC | AAC27923.1 | 3360390 | Clostridium pasteuranum |
| dhaE | AAC27924.1 | 3360391 | Clostridium pasteuranum |
| dhaB | P45514.1 | 1169287 | Citrobacter freundii |
| dhaC | AAB48851.1 | 1229154 | Citrobacter freundii |
| dhaE | AAB48852.1 | 1229155 | Citrobacter freundii |

If a B12-dependent diol dehydratase is utilized, heterologous expression of the corresponding reactivating factor is recommended. B12-dependent diol dehydratases are subject to mechanism-based suicide activation by substrates and some downstream products. Inactivation, caused by a tight association with inactive cobalamin, can be partially overcome by diol dehydratase reactivating factors in an ATP-dependent process. Regeneration of the B12 cofactor requires an additional ATP. Diol dehydratase regenerating factors are two-subunit proteins. Exemplary candidates are found in Klebsiella oxytoca (Mori et al., J Biol. Chem. 272:32034-32041 (1997)), Salmonella typhimurium (Bobik et al., J Bacteriol. 179:6633-6639 (1997); Chen et al., J Bacteriol. 176:5474-5482 (1994)), Lactobacillus collinoides (Sauvageot et al., FEMS Microbiol Lett. 209:69-74 (2002)), Klebsiella pneumonia (World Patent WO 2008/137403).

| Protein | GenBank ID | GI Number | Organism |
|---|---|---|---|
| ddrA | AAC15871 | 3115376 | Klebsiella oxytoca |
| ddrB | AAC15872 | 3115377 | Klebsiella oxytoca |
| pduG | AAB84105 | 16420573 | Salmonella typhimurium |
| pduH | AAD39008 | 16420574 | Salmonella typhimurium |
| pduG | YP_002236779 | 206579698 | Klebsiella pneumonia |
| pduH | YP_002236778 | 206579863 | Klebsiella pneumonia |
| pduG | CAD01092 | 29335724 | Lactobacillus collinoides |
| pduH | AJ297723 | 29335725 | Lactobacillus collinoides |

B12-independent diol dehydratase enzymes are glycyl radicals that utilize S-adenosylmethionine (SAM) as a cofactor and function under strictly anaerobic conditions. The glycerol dehydrogenase and corresponding activating factor of Clostridium butyricum, encoded by dhaB1 and dhaB2, have been well-characterized (O'Brien et al., Biochemistry 43:4635-4645 (2004); Raynaud et al., Proc. Natl. Acad. Sci U.S.A 100:5010-5015 (2003)). This enzyme was recently employed in a 1,3-propanediol overproducing strain of E. coli and was able to achieve very high titers of product (Tang et al., Appl. Environ. Microbiol. 75:1628-1634 (2009)). An additional B12-independent diol dehydratase enzyme and activating factor from Roseburia inulinivorans was shown to catalyze the conversion of 2,3-butanediol to 2-butanone (US 2009/09155870). A B12-independent, oxygen sensitive and membrane bound diol dehydratase from Clostridium glycolycum catalyzes the dehydration of 1,2-ethanediol to acetaldehyde; however the gene has not been identified to date (Hartmanis et al, Arch Biochem Biophys, 245:144-152 (1986)).

| Protein | GenBank ID | GI Number | Organism |
|---|---|---|---|
| dhaB1 | AAM54728.1 | 27461255 | Clostridium butyricum |
| dhaB2 | AAM54729.1 | 27461256 | Clostridium butyricum |
| rdhtA | ABC25539.1 | 83596382 | Roseburia inulinivorans |
| rdhtB | ABC25540.1 | 83596383 | Roseburia inulinivorans |

Dihydroxy-acid dehydratase (DHAD, EC 4.2.1.9) is a B12-independent enzyme participating in branched-chain amino acid biosynthesis. In its native role, it converts 2,3-dihydroxy-3-methylvalerate to 2-keto-3-methyl-valerate, a precursor of isoleucine. In valine biosynthesis the enzyme catalyzes the dehydration of 2,3-dihydroxy-isovalerate to 2-oxoisovalerate. The DHAD from Sulfolobus solfataricus has a broad substrate range and activity of a recombinant enzyme expressed in E. coli was demonstrated on a variety of aldonic acids (KIM et al., J. Biochem. 139:591-596 (2006)). The S. solfataricus enzyme is tolerant of oxygen unlike many diol dehydratase enzymes. The E. coli enzyme, encoded by ilvD, is sensitive to oxygen, which inactivates its iron-sulfur cluster (Flint et al., J. Biol. Chem. 268:14732-14742 (1993)) Similar enzymes have been characterized in Neurospora crassa (Altmiller et al., Arch. Biochem. Biophys. 138:160-170 (1970)), Salmonella typhimurium (Armstrong et al., Biochim. Biophys. Acta 498:282-293 (1977)) and Corynebacterium glutamicum (Holatko et al, J Biotechnol 139:203-10 (2009)). Other groups have shown that the overexpression of one or more Aft proteins or homologs thereof improves DHAD activity (US Patent Application 2011/0183393. In Saccharomyces cerevisiae, the Aft1 and Aft2 proteins are transcriptional activators that regulate numerous proteins related to the acquisition, compartmentalization, and utilization of iron.

| Protein | GenBank ID | GI Number | Organism |
|---|---|---|---|
| ilvD | NP_344419.1 | 15899814 | Sulfolobus solfataricus |
| ilvD | AAT48208.1 | 48994964 | Escherichia coli |
| ilvD | NP_462795.1 | 16767180 | Salmonella typhimurium |
| ilvD | XP_958280.1 | 85090149 | Neurospora crassa |
| ilvD | CAB57218.1 | 6010023 | Corynebacterium glutamicum |
| Aft1 | P22149.2 | 1168370 | Saccharomyces cerevisiae |
| Aft2 | Q08957.1 | 74583775 | Saccharomyces cerevisiae |

### Example XI

### Pathways for Converting Succinyl-CoA to 3-Buten-2-Ol and/or Butadiene

This example describes pathways for converting succinyl-CoA to 3-buten-2-ol, and further to butadiene. The conversion of succinyl-CoA to 3-buten-2-ol is accomplished in five enzymatic steps. Succinyl-CoA and acetyl-CoA are first condensed to 3-oxoadipyl-CoA by 3-oxoadipyl-CoA thiolase, a beta-ketothiolase (Step 6A). The 3-oxoadipyl-CoA product is converted to its corresponding acid by a CoA hydrolase, transferase or synthetase (Step 6B). Decarboxylation of the 3-oxoacid to 4-oxopentanoate (Step 6C), and subsequent reduction by a 4-oxopentanoate reductase yields 4-hydroxypentanoate (Step 6D). Oxidative decarboxylation of 4-hydroxypentanoate yields 3-buten-2-ol (Step 6E). 3-Buten-2-ol is further converted to butadiene via chemical dehydration or by a dehydratase enzyme (Step 5G).

Enzymes and gene candidates for catalyzing but-3-en-2-ol and butadiene pathway reactions are described herein. Enzymes for steps A-F are described above.

### Example XII

### Identification of 3-Buten-2-Ol Regulatory Elements

Organisms that metabolize 3-buten-2-ol or its methylated analog, 2-methyl-3-buten-2-ol, can be examined for regulatory elements responsive to 3-buten-2-ol or 3-buten-2-ol pathway intermediates. For example, the genome of Pseudomonas putida MB-1 encodes an alcohol dehydrogenase and aldehyde dehydrogenase that is induced by 3-methyl-2-buten-3-ol (Malone et al, AEM 65: 2622-30 (1999)). The promoter of these genes can be used in several capacities, such as, being linked to expression of a fluorescent protein or other indicator that can be used to identify when 3-buten-2-ol is produced and in some aspect the quantity of 3-buten-2-ol produced by an organism of the invention.

### Example XIII

### Chemical Dehydration of 1,3-BDO to Butadiene

1,3-Butanediol (also referred to as 13BDO) can be a biosynthetic pathway intermediate to the product butadiene as described herein, or 13BDO can be the biosynthetic product. After biosynthetic production of 13BDO is achieved, access to butadiene can be accomplished by 13BDO isolation, optional purification, and subsequent chemical (or enzymatic) dehydration to butadiene. Provided is a process for the production of butadiene that includes (a) culturing by fermentation in a sufficient amount of nutrients and media a non-naturally occurring microbial organism that produces 13BDO according to any of the methods described herein; and (b) isolating the 13BDO from the fermentation broth; and (c) converting the isolated 13BDO produced by culturing the non-naturally occurring microbial organism to butadiene. Optionally, and preferably, after step (b) and before step (c) the isolated 13BDO is purified by a process comprising one, two, three or four additional purification steps that include one, two or more distillation steps, a salt reduction or removal step, and/or a water reduction or removal step.

In the embodiment where 1,3-BDO is the biosynthetic product, 1,3-BDO can be converted to butadiene by dehydration—two waters are removed. In one embodiment 1,3-BDO is first dehydrated to crotyl alcohol that is then further dehydrated to butadiene.

Following the dehydration step, the resulting butadiene is isolated and purified by a suitable method including those described herein. Un-reacted 13BDO and other byproducts can be recycled to the dehydration step or purged from the process.

### Example XIV

### Chemical Dehydration of Crotyl Alcohol to Butadiene

Crotyl alcohol can be a biosynthetic pathway intermediate to the product butadiene as described herein, or crotyl alcohol can be the biosynthetic product. After biosynthetic production of crotyl alcohol is achieved, access to butadiene can be accomplished by crotyl alcohol isolation, optional purification, and subsequent chemical (or enzymatic) dehydration to butadiene. Provided is a process for the production of butadiene that includes (a) culturing by fermentation in a sufficient amount of nutrients and media a non-naturally occurring microbial organism that produces crotyl alcohol according to any of the methods described herein; and (b) isolating the crotyl alcohol from the fermentation broth; and (c) converting the isolated crotyl alcohol produced by culturing the non-naturally occurring microbial organism to butadiene. Converting the alcohol to butadiene can be performed by dehydration enzymatically or chemically, with or without a catalyst. Optionally, after step (b) and before step (c) the isolated crotyl alcohol is purified by a process comprising one, two, three or four additional purification steps that include one, two or more distillation steps, a salt reduction or removal step, and/or a water reduction or removal step. Following fermentation the crotyl alcohol is isolated from the fermentation broth prior to enzymatic or catalytic dehydration to butadiene. The isolation comprises a distillation step. The normal boiling point of crotyl alcohol is about 122 degrees C., which does not suggest an easy separation from fermentation broth. The preferred isolation process described herein exploits a crotyl alcohol-water azeotrope to facilitate isolation. Its azeotrope with water occurs at approximately 90 to 95 degrees C. It is widely recognized that an azeotrope typically causes complications and challenges for a separations process. Further the presence of impurities and byproducts in the fermentation broth point away from a simple, short isolation process. A simple, short isolation process would be even more avoided for use with a biomass feedstock that contains more and varied impurities and byproducts than a purified sugar feedstock, e.g. dextrose. Despite these complications, the present inventors recognized the presence of the azeotrope and that its presence in the fermentation broth facilitates and simplifies the isolation process. Exploiting this property to provide a simple isolation process is unique for the fermentation production of crotyl alcohol because of the presence of water. Since the azeotrope has a higher relative volatility than water (normal boiling point of water is 100 degrees C.), the azeotropic mixture can be removed directly from the aqueous fermentation broth as the overheads from a distillation column. Water (non-azeotrope), feedstock impurities, microbial biomass, and fermentation byproducts that have lower relative volatilities will be left behind in the distillation column bottoms. Accordingly, the distillation step will be at a temperature that vaporizes the azeotrope and minimizes vaporization of the other materials in the fermentation broth, typically about 90 to 95 degrees C., and in one embodiment can be about 94.2 degrees C.

The isolated crotyl alcohol, for example as an azeotropic mixture with water, can be dehydrated to butadiene in Step (c). In one such embodiment, the crotyl alcohol, e.g. as a crotyl alcohol-water azeotrope, is subjected to a one-step catalytic dehydration to butadiene without any additional drying or purification. Optionally, if a higher purity of crotyl alcohol is preferred for the catalytic dehydration the crotyl alcohol can be dried, for example by passing the azeotropic mixture through a molecular sieve or via azeotropic distillation using a third component such as an organic solvent, e.g., benzene. The dried crotyl alcohol can optionally undergo further refining and purification as needed to obtain a desired purity for catalytic dehydration to butadiene. Alternatively, a purification step can precede a drying step, or can occur at the same time, or where multiple drying and/or purification steps are used they can occur in any order.

The dehydration of alcohols to olefins, specifically butadiene, is known in the art and can include various thermal processes, both catalyzed and non-catalyzed. In some embodiments, a catalyzed thermal dehydration employs a metal oxide catalyst or silica. For example, crotyl alcohol can be dehydrated over bismuth molybdate (Adams, C. R. J. Catal. 10:355-361, 1968) to produce 1,3-butadiene. Also see Winfield, Catalytic Dehydration and Hydration, Chapter 2, in Catalysis Volume VII: Oxidation, Hydration, Dehydration and Cracking Catalysis, 1960, ed. Paul H. Emmett, Reinhold Publishing Corporation, New York, N.Y. USA.

Dehydration can be achieved via activation of the alcohol group and subsequent elimination by standard elimination mechanisms such as E1 or E2 elimination. Activation can be achieved by way of conversion of the alcohol group to a halogen such as iodide, chloride, or bromide. Activation can also be accomplished by way of a sulfonyl, phosphate or other activating functionality that convert the alcohol into a good leaving group. In some embodiments, the activating group is a sulfate or sulfate ester selected from a tosylate, a mesylate, a nosylate, a brosylate, and a triflate. In some embodiments, the leaving group is a phosphate or phosphate ester. In some such embodiments, the dehydrating agent is phosphorus pentoxide.

Dehydration reactions can be carried out in both gas and liquid phases with both heterogeneous and homogeneous catalyst systems in many different reactor configurations. Typically, the catalysts used are stable to the water that is generated by the reaction. The water is usually removed from the reaction zone with the product. The resulting alkene(s) either exit the reactor in the gas or liquid phase (e.g., depending upon the reactor conditions) and are captured by a downstream purification process or are further converted in the reactor to other compounds (such as butadiene or isoprene) as described herein. The water generated by the dehydration reaction exits the reactor with unreacted alcohol and alkene product(s) and is separated by distillation or phase separation. Because water is generated in large quantities in the dehydration step, the dehydration catalysts used are generally tolerant to water and a process for removing the water from substrate and product may be part of any process that contains a dehydration step. For this reason, it is possible to use wet MVC as a substrate for a dehydration reaction and remove this water with the water generated by the dehydration reaction (e.g., using a zeolite catalyst as described U.S. Pat. Nos. 4,698,452 and 4,873,392). Additionally, neutral alumina and zeolites will dehydrate alcohols to alkenes but generally at higher temperatures and pressures than the acidic versions of these catalysts. Dehydration of alcohols, including crotyl alcohol, to butadiene is described in Gustay. Egloff and George. Hulla, Chem. Rev., 1945, 36 (1), pp 63-141.

In a typical process for converting crotyl alcohol into butadiene, crotyl alcohol is passed, either neat or in a solvent and either in presence or absence of steam, over a solid inorganic, organic or metal-containing dehydration catalyst heated to temperatures in the range 40-400° C. inside of the reaction vessel or tube, leading to elimination of water and release of butadiene as a gas, which is condensed (butadiene bp=−4.4° C.) and collected in a reservoir for further processing, storage, or use. Typical catalysts can include bismuth molybdate, phosphate-phosphoric acid, cerium oxide, kaolin-iron oxide, kaolin-phosphoric acid, silica-alumina, and alumina. Typical process throughputs are in the range of 0.1-20,000 kg/h. Typical solvents are toluene, heptane, octane, ethylbenzene, and xylene.

Following the dehydration step, the resulting butadiene is isolated and purified by a suitable method including those described herein. Un-reacted crotyl alcohol and other byproducts can be recycled to the dehydration step or purged from the process.

Accordingly, the route to butadiene via crotyl alcohol isolation has a significant advantage versus the route via 13BDO in part because it requires fewer separation steps and only one versus two dehydrations. More separation steps are required for 13BDO since it is more miscible in water and its normal boiling point is about 205 degrees C. Due to the unique physical properties of crotyl alcohol, the isolation route as described herein allows its fermentation production with low-quality, impure biomass feedstock. Isolating crotyl alcohol from salts and other impurities is not as difficult as for 13BDO since the crotyl-alcohol azeotrope can be distilled directly from the broth leaving a bulk of the impurities behind in the distillation bottoms.

### Example XV

### Chemical Dehydration of 3-Buten-2-Ol to Butadiene

3-Buten-2-ol (also referred to as methyl vinyl carbinol; MVC) can be a biosynthetic pathway intermediate to the product butadiene as described herein, or MVC can be the biosynthetic product. After biosynthetic production of MVC is achieved, access to butadiene can be accomplished by MVC isolation, optional purification, and subsequent chemical (or enzymatic) dehydration to butadiene. Provided is a process for the production of butadiene that includes (a) culturing by fermentation in a sufficient amount of nutrients and media a non-naturally occurring microbial organism that produces MVC according to any of the methods described herein; and (b) isolating the MVC from the fermentation broth; and (c) converting the isolated MVC produced by culturing the non-naturally occurring microbial organism to butadiene. Converting MVC to butadiene can be performed by dehydration enzymatically or chemically, with or without a catalyst. Optionally, after step (b) and before step (c) the isolated MVC is purified by a process comprising one, two, three or four additional purification steps that include one, two or more distillation steps, a salt reduction or removal step, and/or a water reduction or removal step.

Following fermentation as described herein, MVC can be isolated from the fermentation broth prior to catalytic dehydration to butadiene. MVC has a boiling point approximating that of water. The azeotrope of MVC and water occurs at about 87 degrees C. It is widely recognized that an azeotrope typically causes complications and challenges for a separations process. Further the presence of impurities and byproducts in the fermentation broth point away from a simple, short isolation process. A simple, short isolation process would be even more avoided for use with a biomass feedstock that contains more and varied impurities and byproducts than a purified sugar feedstock, e.g. dextrose. Despite these complications, the present inventors recognized the presence of the MVC-water azeotrope and that its presence in the fermentation broth facilitates and simplifies the isolation process. Exploiting this property to provide a simple isolation process is unique for the fermentation production of MVC because of the presence of water. Since the azeotrope has a higher relative volatility than water (normal boiling point of water is 100 degrees C.), the azeotropic mixture can be removed directly from the aqueous fermentation broth as the overheads from a distillation column. Water (non-azeotrope), feedstock impurities, microbial biomass, and fermentation byproducts that have lower relative volatilities will be left behind in the distillation column bottoms.

The isolated MVC, for example as an azeotropic mixture with water, can be dehydrated to butadiene in step (c). In one such embodiment, the MVC, e.g. as a MVC-water azeotrope, is subjected to a one-step catalytic dehydration to butadiene without any additional drying or purification. Optionally, if a higher purity of MVC is preferred for the catalytic dehydration the MVC can be dried, for example by passing the azeotropic mixture through a molecular sieve or via azeotropic distillation using a third component such as an organic solvent, e.g., benzene. The dried MVC can optionally undergo further refining and purification as needed to obtain a desired purity for catalytic dehydration to butadiene. Alternatively, a purification step can precede a drying step, or can occur at the same time, or where multiple drying and/or purification steps are used they can occur in any order.

The dehydration of alcohols to olefins, specifically butadiene, are known in the art and can include various thermal processes, both catalyzed and non-catalyzed. In some embodiments, a catalyzed thermal dehydration employs a metal oxide catalyst or silica. Step (c) of the process, dehydration, can be performed enzymatically or by chemically in the presence of a catalyst. For example, see Winfield, Catalytic Dehydration and Hydration, Chapter 2, in Catalysis Volume VII: Oxidation, Hydration, Dehydration and Cracking Catalysis, 1960, ed. Paul H. Emmett, Reinhold Publishing Corporation, New York, N.Y. USA.

Dehydration can be achieved via activation of the alcohol group and subsequent elimination by standard elimination mechanisms such as E1 or E2 elimination. Activation can be achieved by way of conversion of the alcohol group to a halogen such as iodide, chloride, or bromide. Activation can also be accomplished by way of a sulfonyl, phosphate or other activating functionality that convert the alcohol into a good leaving group. In some embodiments, the activating group is a sulfate or sulfate ester selected from a tosylate, a mesylate, a nosylate, a brosylate, and a triflate. In some embodiments, the leaving group is a phosphate or phosphate ester. In some such embodiments, the dehydrating agent is phosphorus pentoxide.

Dehydration reactions can be carried out in both gas and liquid phases with both heterogeneous and homogeneous catalyst systems in many different reactor configurations. Typically, the catalysts used are stable to the water that is generated by the reaction. The water is usually removed from the reaction zone with the product. The resulting alkene(s) either exit the reactor in the gas or liquid phase (e.g., depending upon the reactor conditions) and are captured by a downstream purification process or are further converted in the reactor to other compounds (such as butadiene or isoprene) as described herein. The water generated by the dehydration reaction exits the reactor with unreacted alcohol and alkene product(s) and is separated by distillation or phase separation. Because water is generated in large quantities in the dehydration step, the dehydration catalysts used are generally tolerant to water and a process for removing the water from substrate and product may be part of any process that contains a dehydration step. For this reason, it is possible to use wet MVC as a substrate for a dehydration reaction and remove this water with the water generated by the dehydration reaction (e.g., using a zeolite catalyst as described U.S. Pat. Nos. 4,698,452 and 4,873,392). Additionally, neutral alumina and zeolites will dehydrate alcohols to alkenes but generally at higher temperatures and pressures than the acidic versions of these catalysts. Dehydration of MVC to butadiene is well known in the art (Gustay. Egloff and George. Hulla, Chem. Rev., 1945, 36 (1), pp 63-141). See also U.S. Pat. No. 2,400,409 entitled “Methods for dehydration of alcohols.”

Following the dehydration step, the resulting butadiene is isolated and purified by a suitable method including those described herein. Un-reacted MVC and other byproducts can be recycled to the dehydration step or purged from the process.

Accordingly, the route to butadiene via MVC isolation has a significant advantage versus the route via 13BDO in part because it requires fewer separation steps and only one versus two dehydrations. More separation steps are required for 13BDO since it is more miscible in water and its normal boiling point is about 205 degrees C. Due to the unique physical properties of MVC, the isolation route as described herein allows its fermentation production with low-quality, impure biomass feedstock. Isolating MVC from salts and other impurities is not as difficult as for 13BDO since the MVC-water azeotrope can be distilled directly from the broth leaving a bulk of the impurities behind in the distillation bottoms.

Throughout this application various publications have been referenced. The disclosures of these publications in their entireties, including GenBank and GI number publications, are hereby incorporated by reference in this application in order to more fully describe the state of the art to which this invention pertains. Although the invention has been described with reference to the examples provided above, it should be understood that various modifications can be made without departing from the spirit of the invention.

1. A non-naturally occurring microbial organism having a formaldehyde fixation pathway and a formate assimilation pathway, wherein said organism comprises at least one exogenous nucleic acid encoding a formaldehyde fixation pathway enzyme expressed in a sufficient amount to produce pyruvate, wherein said formaldehyde fixation pathway comprises:
(1) 1B and 1C; or (2) 1D,
wherein 1B is a 3-hexulose-6-phosphate synthase, wherein 1C is a 6-phospho-3-hexuloisomerase, wherein 1D is a dihydroxyacetone synthase,
wherein said organism comprises at least one exogenous nucleic acid encoding a formate assimilation pathway enzyme expressed in a sufficient amount to produce formaldehyde, pyruvate, or acetyl-CoA, wherein said formate assimilation pathway comprises a pathway selected from:
(3) 1E; (4) 1F, and 1G; (5) 1H, 1I, 1J, and 1K; (6) 1H, 1I, 1J, 1L, 1M, and 1N; (7) 1E, 1H, 1I, 1J, 1L, 1M, and 1N; (8) 1F, 1G, 1H, 1I, 1J, 1L, 1M, and 1N; (9) 1K, 1H, 1I, 1J, 1L, 1M, and 1N; and (10) 1H, 1I, 1J, 1O, and 1P,
wherein 1E is a formate reductase, 1F is a formate ligase, a formate transferase, or a formate synthetase, wherein 1G is a formyl-CoA reductase, wherein 1H is a formyltetrahydrofolate synthetase, wherein 1I is a methenyltetrahydrofolate cyclohydrolase, wherein 1J is a methylenetetrahydrofolate dehydrogenase, wherein 1K is a formaldehyde-forming enzyme or spontaneous, wherein 1L is a glycine cleavage system, wherein 1M is a serine hydroxymethyltransferase, wherein 1N is a serine deaminase, wherein 1O is a methylenetetrahydrofolate reductase, wherein 1P is an acetyl-CoA synthase.

2.-3. (canceled)

4. The non-naturally occurring microbial organism of claim 1, wherein said formate assimilation pathway further comprises:
(1) 1Q; or (2) 1R, and 1S,
wherein 1Q is a pyruvate formate lyase, wherein 1R is a pyruvate dehydrogenase, a pyruvate ferredoxin oxidoreductase, or a pyruvate:NADP+ oxidoreductase, wherein 1S is a formate dehydrogenase.

5. (canceled)

6. The non-naturally occurring microbial organism of claim 1, wherein said organism comprises at least one exogenous nucleic acid encoding a methanol metabolic pathway enzyme expressed in a sufficient amount to produce formaldehyde or produce or enhance the availability of reducing equivalents in the presence of methanol, wherein said methanol metabolic pathway comprises a pathway selected from:
(1) 3J; (2) 3A and 3B; (3) 3A, 3B and 3C; (4) 3J, 3K and 3C; (5) 3J, 3M, and 3N; (6) 3J and 3L; (7) 3A, 3B, 3C, 3D, and 3E; (8) 3A, 3B, 3C, 3D, and 3F; (9) 3J, 3K, 3C, 3D, and 3E; (10) 3J, 3K, 3C, 3D, and 3F; (11) 3J, 3M, 3N, and 3O; (12) 3A, 3B, 3C, 3D, 3E, and 3G; (13) 3A, 3B, 3C, 3D, 3F, and 3G; (14) 3J, 3K, 3C, 3D, 3E, and 3G; (15) 3J, 3K, 3C, 3D, 3F, and 3G; (16) 3J, 3M, 3N, 3O, and 3G; (17) 3A, 3B, 3C, 3D, 3E, and 3I; (18) 3A, 3B, 3C, 3D, 3F, and 3I; (19) 3J, 3K, 3C, 3D, 3E, and 3I; (20) 3J, 3K, 3C, 3D, 3F, and 3I; and (21) 3J, 3M, 3N, 3O, and 3I,
wherein 3A is a methanol methyltransferase, wherein 3B is a methylenetetrahydrofolate reductase, wherein 3C is a methylenetetrahydrofolate dehydrogenase, wherein 3D is a methenyltetrahydrofolate cyclohydrolase, wherein 3E is a formyltetrahydrofolate deformylase, wherein 3F is a formyltetrahydrofolate synthetase, wherein 3G is a formate hydrogen lyase, wherein 3H is a hydrogenase, wherein 3I is a formate dehydrogenase, wherein 3J is a methanol dehydrogenase, wherein 3K is a formaldehyde activating enzyme or spontaneous, wherein 3L is a formaldehyde dehydrogenase, wherein 3M is a S-(hydroxymethyl)glutathione synthase or spontaneous, wherein 3N is a glutathione-dependent formaldehyde dehydrogenase, wherein 3O is a S-formylglutathione hydrolase,

7. (canceled)

8. The non-naturally occurring microbial organism of claim 1, wherein said organism comprises at least one exogenous nucleic acid encoding a methanol oxidation pathway enzyme expressed in a sufficient amount to produce formaldehyde in the presence of methanol, wherein said methanol oxidation pathway comprises 1A, wherein 1A a methanol dehydrogenase.

9-13. (canceled)

14. A non-naturally occurring microbial organism having a butadiene pathway and comprising at least one exogenous nucleic acid encoding a butadiene pathway enzyme expressed in a sufficient amount to produce butadiene, wherein said butadiene pathway comprises a pathway selected from:
(1) 10A, 10D, 10E, 10F, 10G, 10S, 15A, 15B, 15C, and 15G; (2) 10A, 10D, 10I, 10G, 10S, 15A, 15B, 15C, and 15G; (3) 10A, 10D, 10K, 10S, 15A, 15B, 15C, and 15G; (4) 10A, 10H, 10F, 10G, 10S, 15A, 15B, 15C, and 15G; (5) 10A, 10J, 10G, 10S, 15A, 15B, 15C, and 15G; (6) 10A, 10J, 10R, 10AA, 15A, 15B, 15C, and 15G; (7) 10A, 10H, 10F, 10R, 10AA, 15A, 15B, 15C, and 15G; (8) 10A, 10H, 10Q, 10Z, 10AA, 15A, 15B, 15C, and 15G; (9) 10A, 10D, 10I, 10R, 10AA, 15A, 15B, 15C, and 15G; (10) 10A, 10D, 10E, 10F, 10R, 10AA, 15A, 15B, 15C, and 15G; (11) 10A, 10D, 10E, 10Q, 10Z, 10AA, 15A, 15B, 15C, and 15G; (12) 10A, 10D, 10P, 10N, 10AA, 15A, 15B, 15C, and 15G; (13) 10A, 10D, 10P, 10Y, 10Z, 10AA, 15A, 15B, 15C, and 15G; (14) 10A, 10B, 10M, 10AA, 15A, 15B, 15C, and 15G; (15) 10A, 10B, 10L, 10Z, 10AA, 15A, 15B, 15C, and 15G; (16) 10A, 10B, 10X, 10N, 10AA, 15A, 15B, 15C, and 15G; (17) 10A, 10B, 10X, 10Y, 10Z, 10AA, 15A, 15B, 15C, and 15G; (18) 10A, 10D, 10P, 10O, 15A, 15B, 15C, and 15G; (19) 10A, 10B, 10X, 10O, 15A, 15B, 15C, and 15G; (20) 10A, 10D, 10E, 10F, 10R, 10AA, 15A, 15B, 15C, and 15G; (21) 10A, 10D, 10E, 10F, 10G, 10S, 15A, 15B, 15C, and 15G; (22) 10A, 10B, 10C, 10AE, 10AB, 10Y, 10Z, 10AA, 15A, 15B, 15C, and 15G; (23) 10A, 10B, 10C, 10AE, 10AB, 10N, 10AA, 15A, 15B, 15C, and 15G; (24) 10A, 10B, 10C, 10AE, 10AB, 10O, 15A, 15B, 15C, and 15G; (25) 10AU, 10AB, 10Y, 10Z, 10AA, 15A, 15B, 15C, and 15G; (26) 10AU, 10AB, 10N, 10AA, 15A, 15B, 15C, and 15G; (27) 10AU, 10AB, 10O, 15A, 15B, 15C, and 15G; (28) 1T, 10AS, 10E, 10F, 10G, 10S, 15A, 15B, 15C, and 15G; (29) 1T, 10AS, 10I, 10G, 10S, 15A, 15B, 15C, and 15G; (30) 1T, 10AS, 10K, 10S, 15A, 15B, 15C, and 15G; (31) 1T, 10AS, 10I, 10R, 10AA, 15A, 15B, 15C, and 15G; (32) 1T, 10AS, 10E, 10F, 10R, 10AA, 15A, 15B, 15C, and 15G; (33) 1T, 10AS, 10E, 10Q, 10Z, 10AA, 15A, 15B, 15C, and 15G; (34) 1T, 10AS, 10P, 10N, 10AA, 15A, 15B, 15C, and 15G; (35) 1T, 10AS, 10P, 10Y, 10Z, 10AA, 15A, 15B, 15C, and 15G; (36) 1T, 10AS, 10P, 10O, 15A, 15B, 15C, and 15G; (37) 1T, 10AS, 10E, 10F, 10R, 10AA, 15A, 15B, 15C, and 15G; (38) 10AT, 10E, 10F, 10G, 10S, 15A, 15B, 15C, and 15G; (39) 10AT, 10I, 10G, 10S, 15A, 15B, 15C, and 15G; (40) 10AT, 10K, 10S, 15A, 15B, 15C, and 15G; (41) 10AT, 10I, 10R, 10AA, 15A, 15B, 15C, and 15G; (42) 10AT, 10E, 10F, 10R, 10AA, 15A, 15B, 15C, and 15G; (43) 10AT, 10E, 10Q, 10Z, 10AA, 15A, 15B, 15C, and 15G; (44) 10AT, 10P, 10N, 10AA, 15A, 15B, 15C, and 15G; (45) 10AT, 10P, 10Y, 10Z, 10AA, 15A, 15B, 15C, and 15G; (46) 10AT, 10P, 10O, 15A, 15B, 15C, and 15G; (47) 10AT, 10E, 10F, 10R, 10AA, 15A, 15B, 15C, and 15G; (48) 10A, 10D, 10E, 10F, 10G, 10S, 15D, and 15G; (49) 10A, 10D, 10I, 10G, 10S, 15D, and 15G; (50) 10A, 10D, 10K, 10S, 15D, and 15G; (51) 10A, 10H, 10F, 10G, 10S, 15D, and 15G; (52) 10A, 10J, 10G, 10S, 15D, and 15G; (53) 10A, 10J, 10R, 10AA, 15D, and 15G; (54) 10A, 10H, 10F, 10R, 10AA, 15D, and 15G; (55) 10A, 10H, 10Q, 10Z, 10AA, 15D, and 15G; (56) 10A, 10D, 10I, 10R, 10AA, 15D, and 15G; (57) 10A, 10D, 10E, 10F, 10R, 10AA, 15D, and 15G; (58) 10A, 10D, 10E, 10Q, 10Z, 10AA, 15D, and 15G; (59) 10A, 10D, 10P, 10N, 10AA, 15D, and 15G; (60) 10A, 10D, 10P, 10Y, 10Z, 10AA, 15D, and 15G; (61) 10A, 10B, 10M, 10AA, 15D, and 15G; (62) 10A, 10B, 10L, 10Z, 10AA, 15D, and 15G; (63) 10A, 10B, 10X, 10N, 10AA, 15D, and 15G; (64) 10A, 10B, 10X, 10Y, 10Z, 10AA, 15D, and 15G; (65) 10A, 10D, 10P, 10O, 15D, and 15G; (66) 10A, 10B, 10X, 10O, 15D, and 15G; (67) 10A, 10D, 10E, 10F, 10R, 10AA, 15D, and 15G; (68) 10A, 10D, 10E, 10F, 10G, 10S, 15D, and 15G; (69) 10A, 10B, 10C, 10AE, 10AB, 10Y, 10Z, 10AA, 15D, and 15G; (70) 10A, 10B, 10C, 10AE, 10AB, 10N, 10AA, 15D, and 15G; (71) 10A, 10B, 10C, 10AE, 10AB, 10O, 15D, and 15G; (72) 10AU, 10AB, 10Y, 10Z, 10AA, 15D, and 15G; (73) 10AU, 10AB, 10N, 10AA, 15D, and 15G; (74) 10AU, 10AB, 10O, 15D, and 15G; (75) 1T, 10AS, 10E, 10F, 10G, 10S, 15D, and 15G; (76) 1T, 10AS, 10I, 10G, 10S, 15D, and 15G; (77) 1T, 10AS, 10K, 10S, 15D, and 15G; (78) 1T, 10AS, 10I, 10R, 10AA, 15D, and 15G; (79) 1T, 10AS, 10E, 10F, 10R, 10AA, 15D, and 15G; (80) 1T, 10AS, 10E, 10Q, 10Z, 10AA, 15D, and 15G; (81) 1T, 10AS, 10P, 10N, 10AA, 15D, and 15G; (82) 1T, 10AS, 10P, 10Y, 10Z, 10AA, 15D, and 15G; (83) 1T, 10AS, 10P, 10O, 15D, and 15G; (84) 1T, 10AS, 10E, 10F, 10R, 10AA, 15D, and 15G; (85) 10AT, 10E, 10F, 10G, 10S, 15D, and 15G; (86) 10AT, 10I, 10G, 10S, 15D, and 15G; (87) 10AT, 10K, 10S, 15D, and 15G; (88) 10AT, 10I, 10R, 10AA, 15D, and 15G; (89) 10AT, 10E, 10F, 10R, 10AA, 15D, and 15G; (90) 10AT, 10E, 10Q, 10Z, 10AA, 15D, and 15G; (91) 10AT, 10P, 10N, 10AA, 15D, and 15G; (92) 10AT, 10P, 10Y, 10Z, 10AA, 15D, and 15G; (93) 10AT, 10P, 10O, 15D, and 15G; (94) 10AT, 10E, 10F, 10R, 10AA, 15D, and 15G; (95) 10A, 10D, 10E, 10F, 10G, 10S, 15E, 15C, and 15G; (96) 10A, 10D, 10I, 10G, 10S, 15E, 15C, and 15G; (97) 10A, 10D, 10K, 10S, 15E, 15C, and 15G; (98) 10A, 10H, 10F, 10G, 10S, 15E, 15C, and 15G; (99) 10A, 10J, 10G, 10S, 15E, 15C, and 15G; (100) 10A, 10J, 10R, 10AA, 15E, 15C, and 15G; (101) 10A, 10H, 10F, 10R, 10AA, 15E, 15C, and 15G; (102) 10A, 10H, 10Q, 10Z, 10AA, 15E, 15C, and 15G; (103) 10A, 10D, 10I, 10R, 10AA, 15E, 15C, and 15G; (104) 10A, 10D, 10E, 10F, 10R, 10AA, 15E, 15C, and 15G; (105) 10A, 10D, 10E, 10Q, 10Z, 10AA, 15E, 15C, and 15G; (106) 10A, 10D, 10P, 10N, 10AA, 15E, 15C, and 15G; (107) 10A, 10D, 10P, 10Y, 10Z, 10AA, 15E, 15C, and 15G; (108) 10A, 10B, 10M, 10AA, 15E, 15C, and 15G; (109) 10A, 10B, 10L, 10Z, 10AA, 15E, 15C, and 15G; (110) 10A, 10B, 10X, 10N, 10AA, 15E, 15C, and 15G; (111) 10A, 10B, 10X, 10Y, 10Z, 10AA, 15E, 15C, and 15G; (112) 10A, 10D, 10P, 10O, 15E, 15C, and 15G; (113) 10A, 10B, 10X, 10O, 15E, 15C, and 15G; (114) 10A, 10D, 10E, 10F, 10R, 10AA, 15E, 15C, and 15G; (115) 10A, 10D, 10E, 10F, 10G, 10S, 15E, 15C, and 15G; (116) 10A, 10B, 10C, 10AE, 10AB, 10Y, 10Z, 10AA, 15E, 15C, and 15G; (117) 10A, 10B, 10C, 10AE, 10AB, 10N, 10AA, 15E, 15C, and 15G; (118) 10A, 10B, 10C, 10AE, 10AB, 10O, 15E, 15C, and 15G; (119) 10AU, 10AB, 10Y, 10Z, 10AA, 15E, 15C, and 15G; (120) 10AU, 10AB, 10N, 10AA, 15E, 15C, and 15G; (121) 10AU, 10AB, 10O, 15E, 15C, and 15G; (122) 1T, 10AS, 10E, 10F, 10G, 10S, 15E, 15C, and 15G; (123) 1T, 10AS, 10I, 10G, 10S, 15E, 15C, and 15G; (124) 1T, 10AS, 10K, 10S, 15E, 15C, and 15G; (125) 1T, 10AS, 10I, 10R, 10AA, 15E, 15C, and 15G; (126) 1T, 10AS, 10E, 10F, 10R, 10AA, 15E, 15C, and 15G; (127) 1T, 10AS, 10E, 10Q, 10Z, 10AA, 15E, 15C, and 15G; (128) 1T, 10AS, 10P, 10N, 10AA, 15E, 15C, and 15G; (129) 1T, 10AS, 10P, 10Y, 10Z, 10AA, 15E, 15C, and 15G; (130) 1T, 10AS, 10P, 10O, 15E, 15C, and 15G; (131) 1T, 10AS, 10E, 10F, 10R, 10AA, 15E, 15C, and 15G; (132) 10AT, 10E, 10F, 10G, 10S, 15E, 15C, and 15G; (133) 10AT, 10I, 10G, 10S, 15E, 15C, and 15G; (134) 10AT, 10K, 10S, 15E, 15C, and 15G; (135) 10AT, 10I, 10R, 10AA, 15E, 15C, and 15G; (136) 10AT, 10E, 10F, 10R, 10AA, 15E, 15C, and 15G; (137) 10AT, 10E, 10Q, 10Z, 10AA, 15E, 15C, and 15G; (138) 10AT, 10P, 10N, 10AA, 15E, 15C, and 15G; (139) 10AT, 10P, 10Y, 10Z, 10AA, 15E, 15C, and 15G; (140) 10AT, 10P, 10O, 15E, 15C, and 15G; (141) 10AT, 10E, 10F, 10R, 10AA, 15E, 15C, and 15G; (142) 10A, 10D, 10E, 10F, 10G, 10S, 15A, 15F, and 15G; (143) 10A, 10D, 10I, 10G, 10S, 15A, 15F, and 15G; (144) 10A, 10D, 10K, 10S, 15A, 15F, and 15G; (145) 10A, 10H, 10F, 10G, 10S, 15A, 15F, and 15G; (146) 10A, 10J, 10G, 10S, 15A, 15F, and 15G; (147) 10A, 10J, 10R, 10AA, 15A, 15F, and 15G; (148) 10A, 10H, 10F, 10R, 10AA, 15A, 15F, and 15G; (149) 10A, 10H, 10Q, 10Z, 10AA, 15A, 15F, and 15G; (150) 10A, 10D, 10I, 10R, 10AA, 15A, 15F, and 15G; (151) 10A, 10D, 10E, 10F, 10R, 10AA, 15A, 15F, and 15G; (152) 10A, 10D, 10E, 10Q, 10Z, 10AA, 15A, 15F, and 15G; (153) 10A, 10D, 10P, 10N, 10AA, 15A, 15F, and 15G; (154) 10A, 10D, 10P, 10Y, 10Z, 10AA, 15A, 15F, and 15G; (155) 10A, 10B, 10M, 10AA, 15A, 15F, and 15G; (156) 10A, 10B, 10L, 10Z, 10AA, 15A, 15F, and 15G; (157) 10A, 10B, 10X, 10N, 10AA, 15A, 15F, and 15G; (158) 10A, 10B, 10X, 10Y, 10Z, 10AA, 15A, 15F, and 15G; (159) 10A, 10D, 10P, 10O, 15A, 15F, and 15G; (160) 10A, 10B, 10X, 10O, 15A, 15F, and 15G; (161) 10A, 10D, 10E, 10F, 10R, 10AA, 15A, 15F, and 15G; (162) 10A, 10D, 10E, 10F, 10G, 10S, 15A, 15F, and 15G; (163) 10A, 10B, 10C, 10AE, 10AB, 10Y, 10Z, 10AA, 15A, 15F, and 15G; (164) 10A, 10B, 10C, 10AE, 10AB, 10N, 10AA, 15A, 15F, and 15G; (165) 10A, 10B, 10C, 10AE, 10AB, 10O, 15A, 15F, and 15G; (166) 10AU, 10AB, 10Y, 10Z, 10AA, 15A, 15F, and 15G; (167) 10AU, 10AB, 10N, 10AA, 15A, 15F, and 15G; (168) 10AU, 10AB, 10O, 15A, 15F, and 15G; (169) 1T, 10AS, 10E, 10F, 10G, 10S, 15A, 15F, and 15G; (170) 1T, 10AS, 10I, 10G, 10S, 15A, 15F, and 15G; (171) 1T, 10AS, 10K, 10S, 15A, 15F, and 15G; (172) 1T, 10AS, 10I, 10R, 10AA, 15A, 15F, and 15G; (173) 1T, 10AS, 10E, 10F, 10R, 10AA, 15A, 15F, and 15G; (174) 1T, 10AS, 10E, 10Q, 10Z, 10AA, 15A, 15F, and 15G; (175) 1T, 10AS, 10P, 10N, 10AA, 15A, 15F, and 15G; (176) 1T, 10AS, 10P, 10Y, 10Z, 10AA, 15A, 15F, and 15G; (177) 1T, 10AS, 10P, 10O, 15A, 15F, and 15G; (178) 1T, 10AS, 10E, 10F, 10R, 10AA, 15A, 15F, and 15G; (179) 10AT, 10E, 10F, 10G, 10S, 15A, 15F, and 15G; (180) 10AT, 10I, 10G, 10S, 15A, 15F, and 15G; (181) 10AT, 10K, 10S, 15A, 15F, and 15G; (182) 10AT, 10I, 10R, 10AA, 15A, 15F, and 15G; (183) 10AT, 10E, 10F, 10R, 10AA, 15A, 15F, and 15G; (184) 10AT, 10E, 10Q, 10Z, 10AA, 15A, 15F, and 15G; (185) 10AT, 10P, 10N, 10AA, 15A, 15F, and 15G; (186) 10AT, 10P, 10Y, 10Z, 10AA, 15A, 15F, and 15G; (187) 10AT, 10P, 10O, 15A, 15F, and 15G; (188) 10AT, 10E, 10F, 10R, 10AA, 15A, 15F, and 15G; (189) 14A, 14B, 14C, 14D, 14E, 13A, and 13B; (190) 15A, 15B, 15C, and 15G; (191) 15D, and 15G; (192) 15E, 15C, and 15G; (193) 15A, 15F, and 15G; (194) 16A, 16B, 16C, 16D, and 16E; (195) 17A, 17B, 17C, 17D, and 17G; (196) 17A, 17E, 17F, 17D, and 17G; (197) 17A, 17B, 17C, 17H, 17I, 17J, and 17G; (198) 18A, 18B, 18C, 18D, 18E, and 18F; (199) 13A, and 13B; and (200) 17A, 17E, 17F, 17H, 17I, 17J, and 17G,
wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AE is a crotonyl-CoA:ACP transferase, wherein 10AS is an acetoacetyl-CoA synthase, wherein 10AT is an acetyl-CoA:acetyl-CoA acyltransferase, wherein 10AU is a 4-hydroxybutyryl-CoA dehydratase, wherein 13A is a 2-butanol desaturase, wherein 13B is a 3-buten-2-ol dehydratase, wherein 14A is an acetolactate synthase, wherein 14B is an acetolactate decarboxylase, wherein 14C is a butanediol dehydrogenase, wherein 14D is a butanediol dehydratase, wherein 14E is a butanol dehydrogenase, wherein 15A is a 1,3-butanediol kinase, wherein 15B is a 3-hydroxybutyrylphosphate kinase, 15C is a 3-hydroxybutyryldiphosphate lyase, wherein 15D is a 1,3-butanediol diphosphokinase, wherein 15E is a 1,3-butanediol dehydratase, wherein 15F is a 3-hydroxybutyrylphosphate lyase, wherein 15G is a 3-buten-2-ol dehydratase, wherein 16A is a 3-oxopent-4-enoyl-CoA thiolase, wherein 16B is a 3-oxopent-4-enoyl-CoA hydrolase, synthetase or transferase, wherein 16C is a 3-oxopent-4-enoate decarboxylase or spontaneous, wherein 16D is a 3-buten-2-one reductase, wherein 16E is a 3-buten-2-ol dehydratase, wherein 17A is a 3-oxo-4-hydroxypentanoyl-CoA thiolase, wherein 17B is a 3-oxo-4-hydroxypentanoyl-CoA transferase, synthetase or hydrolase, wherein 17C is a 3-oxo-4-hydroxypentanoate reductase, wherein 17D is a 3,4-dihydroxypentanoate decarboxylase, wherein 17E is a 3-oxo-4-hydroxypentanoyl-CoA reductase, wherein 17F is a 3,4-dihydroxypentanoyl-CoA transferase, synthetase or hydrolase, wherein 17G is a 3-buten-2-ol dehydratase, wherein 17H is a 3,4-dihydroxypentanoate dehydratase, wherein 17I is a 4-oxopentanoate reductase, wherein 17J is a 4-hyd4-oxoperoxypentanoate decarboxylase, wherein 18A is a 3-oxoadipyl-CoA thiolase, wherein 18B is a 3-oxoadipyl-CoA transferase, synthetase or hydrolase, wherein 18C is a 3-oxoadipate decarboxylase or spontaneous, wherein 18D is a 4-oxopentanoate reductase, wherein 18E is a 4-hydroxypentanoate decarboxylase, wherein 18F is a 3-buten-2-ol dehydratase.

15-16. (canceled)

17. The non-naturally occurring microbial organism of claim 1, wherein said microbial organism further comprises a formaldehyde fixation pathway comprising at least one exogenous nucleic acid encoding a formaldehyde fixation pathway enzyme expressed in a sufficient amount to produce pyruvate, wherein said formaldehyde fixation pathway comprises:
(1) 1B and 1C; or (2) 1D,
wherein 1B is a 3-hexulose-6-phosphate synthase, wherein 1C is a 6-phospho-3-hexuloisomerase, wherein 1D is a dihydroxyacetone synthase.

18-27. (canceled)

28. The non-naturally occurring microbial organism of claim 1, wherein said organism further comprises a butadiene pathway and comprising at least one exogenous nucleic acid encoding a butadiene pathway enzyme expressed in a sufficient amount to produce butadiene, wherein said butadiene pathway comprises a pathway selected from:
(1) 10A, 10J, 10R, 10AD, 10AH, 11A, 11B, and 11C; (2) 10A, 10H, 10F, 10R, 10AD, 10AH, 11A, 11B, and 11C; (3) 10A, 10H, 10Q, 10Z, 10AD, 10AH, 11A, 11B, and 11C; (4) 10A, 10H, 10Q, 10AC, 10AG, 10AH, 11A, 11B, and 11C; (5) 10A, 10D, 10I, 10R, 10AD, 10AH, 11A, 11B, and 11C; (6) 10A, 10D, 10E, 10F, 10R, 10AD, 10AH, 11A, 11B, and 11C; (7) 10A, 10D, 10E, 10Q, 10Z, 10AD, 10AH, 11A, 11B, and 11C; (8) 10A, 10D, 10E, 10Q, 10AC, 10AG, 10AH, 11A, 11B, and 11C; (9) 10A, 10D, 10P, 10N, 10AD, 10AH, 11A, 11B, and 11C; (10) 10A, 10D, 10P, 10Y, 10Z, 10AD, 10AH, 11A, 11B, and 11C; (11) 10A, 10D, 10P, 10Y, 10AC, 10AG, 10AH, 11A, 11B, and 11C; (12) 10A, 10D, 10P, 10AB, 10V, 10AH, 11A, 11B, and 11C; (13) 10A, 10D, 10P, 10AB, 10AF, 10AG, 10AH, 11A, 11B, and 11C; (14) 10A, 10B, 10M, 10AD, 10AH, 11A, 11B, and 11C; (15) 10A, 10B, 10L, 10Z, 10AD, 10AH, 11A, 11B, and 11C; (16) 10A, 10B, 10L, 10AC, 10AG, 10AH, 11A, 11B, and 11C; (17) 10A, 10B, 10X, 10Y, 10Z, 10AD, 10AH, 11A, 11B, and 11C; (18) 10A, 10B, 10X, 10Y, 10AC, 10AG, 10AH, 11A, 11B, and 11C; (19) 10A, 10B, 10X, 10AB, 10V, 10AH, 11A, 11B, and 11C; (20) 10A, 10B, 10X, 10AB, 10AF, 10AG, 10AH, 11A, 11B, and 11C; (21) 10A, 10B, 10C, 10U, 10AH, 11A, 11B, and 11C; (22) 10A, 10B, 10C, 10T, 10AG, 10AH, 11A, 11B, and 11C; (23) 10A, 10B, 10C, 10AE, 10AF, 10AG, 10AH, 11A, 11B, and 11C; (24) 10A, 10D, 10P, 10AB, 10W, 11A, 11B, and 11C; (25) 10A, 10B, 10X, 10AB, 10W, 11A, 11B, and 11C; (26) 10A, 10B, 10C, 10AE, 10W, 11A, 11B, and 11C; (27) 10A, 10B, 10C, 10AE, 10V, 10AH, 11A, 11B, and 11C (28) 10A, 10J, 10R, 10AD, 10AH, 11D, and 11C; (29) 10A, 10H, 10F, 10R, 10AD, 10AH, 11D, and 11C; (30) 10A, 10H, 10Q, 10Z, 10AD, 10AH, 11D, and 11C; (31) 10A, 10H, 10Q, 10AC, 10AG, 10AH, 11D, and 11C; (32) 10A, 10D, 10I, 10R, 10AD, 10AH, 11D, and 11C; (33) 10A, 10D, 10E, 10F, 10R, 10AD, 10AH, 11D, and 11C; (34) 10A, 10D, 10E, 10Q, 10Z, 10AD, 10AH, 11D, and 11C; (35) 10A, 10D, 10E, 10Q, 10AC, 10AG, 10AH, 11D, and 11C; (36) 10A, 10D, 10P, 10N, 10AD, 10AH, 11D, and 11C; (37) 10A, 10D, 10P, 10Y, 10Z, 10AD, 10AH, 11D, and 11C; (38) 10A, 10D, 10P, 10Y, 10AC, 10AG, 10AH, 11D, and 11C; (39) 10A, 10D, 10P, 10AB, 10V, 10AH, 11D, and 11C; (40) 10A, 10D, 10P, 10AB, 10AF, 10AG, 10AH, 11D, and 11C; (41) 10A, 10B, 10M, 10AD, 10AH, 11D, and 11C; (42) 10A, 10B, 10L, 10Z, 10AD, 10AH, 11D, and 11C; (43) 10A, 10B, 10L, 10AC, 10AG, 10AH, 11D, and 11C; (44) 10A, 10B, 10X, 10Y, 10Z, 10AD, 10AH, 11D, and 11C; (45) 10A, 10B, 10X, 10Y, 10AC, 10AG, 10AH, 11D, and 11C; (46) 10A, 10B, 10X, 10AB, 10V, 10AH, 11D, and 11C; (47) 10A, 10B, 10X, 10AB, 10AF, 10AG, 10AH, 11D, and 11C; (48) 10A, 10B, 10C, 10U, 10AH, 11D, and 11C; (49) 10A, 10B, 10C, 10T, 10AG, 10AH, 11D, and 11C; (50) 10A, 10B, 10C, 10AE, 10AF, 10AG, 10AH, 11D, and 11C; (51) 10A, 10D, 10P, 10AB, 10W, 11D, and 11C; (52) 10A, 10B, 10X, 10AB, 10W, 11D, and 11C; (53) 10A, 10B, 10C, 10AE, 10W, 11D, and 11C; (54) 10A, 10B, 10C, 10AE, 10V, 10AH, 11D, and 11C; (55) 10I, 10R, 10AD, 10AH, 11A, 11B, and 11C; (56) 10E, 10F, 10R, 10AD, 10AH, 11A, 11B, and 11C; (57) 10E, 10Q, 10Z, 10AD, 10AH, 11A, 11B, and 11C; (58) 10E, 10Q, 10AC, 10AG, 10AH, 11A, 11B, and 11C; (59) 10P, 10N, 10AD, 10AH, 11A, 11B, and 11C; (60) 10P, 10Y, 10Z, 10AD, 10AH, 11A, 11B, and 11C; (61) 10P, 10Y, 10AC, 10AG, 10AH, 11A, 11B, and 11C; (62) 10P, 10AB, 10V, 10AH, 11A, 11B, and 11C; (63) 10P, 10AB, 10AF, 10AG, 10AH, 11A, 11B, and 11C; (64) 10P, 10AB, 10W, 11A, 11B, and 11C; (65) 10I, 10R, 10AD, 10AH, 11D, and 11C; (66) 10E, 10F, 10R, 10AD, 10AH, 11D, and 11C; (67) 10E, 10Q, 10Z, 10AD, 10AH, 11D, and 11C; (68) 10E, 10Q, 10AC, 10AG, 10AH, 11D, and 11C; (69) 10P, 10N, 10AD, 10AH, 11D, and 11C; (70) 10P, 10Y, 10Z, 10AD, 10AH, 11D, and 11C; (71) 10P, 10Y, 10AC, 10AG, 10AH, 11D, and 11C; (72) 10P, 10AB, 10V, 10AH, 11D, and 11C; (73) 10P, 10AB, 10AF, 10AG, 10AH, 11D, and 11C; (74) 10P, 10AB, 10W, 11D, and 11C; (75) 1T, 10AS, 10I, 10R, 10AD, 10AH, 11A, 11B, and 11C; (76) 1T, 10AS, 10E, 10F, 10R, 10AD, 10AH, 11A, 11B, and 11C; (77) 1T, 10AS, 10E, 10Q, 10Z, 10AD, 10AH, 11A, 11B, and 11C; (78) 1T, 10AS, 10E, 10Q, 10AC, 10AG, 10AH, 11A, 11B, and 11C; (79) 1T, 10AS, 10P, 10N, 10AD, 10AH, 11A, 11B, and 11C; (80) 1T, 10AS, 10P, 10Y, 10Z, 10AD, 10AH, 11A, 11B, and 11C; (81) 1T, 10AS, 10P, 10Y, 10AC, 10AG, 10AH, 11A, 11B, and 11C; (82) 1T, 10AS, 10P, 10AB, 10V, 10AH, 11A, 11B, and 11C; (83) 1T, 10AS, 10P, 10AB, 10AF, 10AG, 10AH, 11A, 11B, and 11C; (84) 1T, 10AS, 10P, 10AB, 10W, 11A, 11B, and 11C; (85) 1T, 10AS, 10I, 10R, 10AD, 10AH, 11D, and 11C; (86) 1T, 10AS, 10E, 10F, 10R, 10AD, 10AH, 11D, and 11C; (87) 1T, 10AS, 10E, 10Q, 10Z, 10AD, 10AH, 11D, and 11C; (88) 1T, 10AS, 10E, 10Q, 10AC, 10AG, 10AH, 11D, and 11C; (89) 1T, 10AS, 10P, 10N, 10AD, 10AH, 11D, and 11C; (90) 1T, 10AS, 10P, 10Y, 10Z, 10AD, 10AH, 11D, and 11C; (91) 1T, 10AS, 10P, 10Y, 10AC, 10AG, 10AH, 11D, and 11C; (92) 1T, 10AS, 10P, 10AB, 10V, 10AH, 11D, and 11C; (93) 1T, 10AS, 10P, 10AB, 10AF, 10AG, 10AH, 11D, and 11C; (94) 1T, 10AS, 10P, 10AB, 10W, 11D, and 11C; (95) 10AT, 10I, 10R, 10AD, 10AH, 11A, 11B, and 11C; (96) 10AT, 10E, 10F, 10R, 10AD, 10AH, 11A, 11B, and 11C; (97) 10AT, 10E, 10Q, 10Z, 10AD, 10AH, 11A, 11B, and 11C; (98) 10AT, 10E, 10Q, 10AC, 10AG, 10AH, 11A, 11B, and 11C; (99) 10AT, 10P, 10N, 10AD, 10AH, 11A, 11B, and 11C; (100) 10AT, 10P, 10Y, 10Z, 10AD, 10AH, 11A, 11B, and 11C; (101) 10AT, 10P, 10Y, 10AC, 10AG, 10AH, 11A, 11B, and 11C; (102) 10AT, 10P, 10AB, 10V, 10AH, 11A, 11B, and 11C; (103) 10AT, 10P, 10AB, 10AF, 10AG, 10AH, 11A, 11B, and 11C; (104) 10AT, 10P, 10AB, 10W, 11A, 11B, and 11C; (105) 10AT, 10I, 10R, 10AD, 10AH, 11D, and 11C; (106) 10AT, 10E, 10F, 10R, 10AD, 10AH, 11D, and 11C; (107) 10AT, 10E, 10Q, 10Z, 10AD, 10AH, 11D, and 11C; (108) 10AT, 10E, 10Q, 10AC, 10AG, 10AH, 11D, and 11C; (109) 10AT, 10P, 10N, 10AD, 10AH, 11D, and 11C; (110) 10AT, 10P, 10Y, 10Z, 10AD, 10AH, 11D, and 11C; (111) 10AT, 10P, 10Y, 10AC, 10AG, 10AH, 11D, and 11C; (112) 10AT, 10P, 10AB, 10V, 10AH, 11D, and 11C; (113) 10AT, 10P, 10AB, 10AF, 10AG, 10AH, 11D, and 11C; (114) 10AT, 10P, 10AB, 10W, 11D, and 11C; (115) 10AU, 10AF, 10AG, 10AH, 11A, 11B, and 11C; (116) 10AU, 10W, 11A, 11B, and 11C; (117) 10AU, 10V, 10AH, 11A, 11B, and 11C; (118) 10AU, 10AF, 10AG, 10AH, 11D, and 11C; (119) 10AU, 10W, 11D, and 11C; (120) 10AU, 10V, 10AH, 11D, and 11C; (121) 10A, 10J, 10R, 10AD, 10AH, and 11E; (122) 10A, 10H, 10F, 10R, 10AD, 10AH, and 11E; (123) 10A, 10H, 10Q, 10Z, 10AD, 10AH, and 11E; (124) 10A, 10H, 10Q, 10AC, 10AG, 10AH, and 11E; (125) 10A, 10D, 10I, 10R, 10AD, 10AH, and 11E; (126) 10A, 10D, 10E, 10F, 10R, 10AD, 10AH, and 11E; (127) 10A, 10D, 10E, 10Q, 10Z, 10AD, 10AH, and 11E; (128) 10A, 10D, 10E, 10Q, 10AC, 10AG, 10AH, and 11E; (129) 10A, 10D, 10P, 10N, 10AD, 10AH, and 11E; (130) 10A, 10D, 10P, 10Y, 10Z, 10AD, 10AH, and 11E; (131) 10A, 10D, 10P, 10Y, 10AC, 10AG, 10AH, and 11E; (132) 10A, 10D, 10P, 10AB, 10V, 10AH, and 11E; (133) 10A, 10D, 10P, 10AB, 10AF, 10AG, 10AH, and 11E; (134) 10A, 10B, 10M, 10AD, 10AH, and 11E; (135) 10A, 10B, 10L, 10Z, 10AD, 10AH, and 11E; (136) 10A, 10B, 10L, 10AC, 10AG, 10AH, and 11E; (137) 10A, 10B, 10X, 10Y, 10Z, 10AD, 10AH, and 11E; (138) 10A, 10B, 10X, 10Y, 10AC, 10AG, 10AH, and 11E; (139) 10A, 10B, 10X, 10AB, 10V, 10AH, and 11E; (140) 10A, 10B, 10X, 10AB, 10AF, 10AG, 10AH, and 11E; (141) 10A, 10B, 10C, 10U, 10AH, and 11E; (142) 10A, 10B, 10C, 10T, 10AG, 10AH, and 11E; (143) 10A, 10B, 10C, 10AE, 10AF, 10AG, 10AH, and 11E; (144) 10A, 10D, 10P, 10AB, 10W, and 11E; (145) 10A, 10B, 10X, 10AB, 10W, and 11E; (146) 10A, 10B, 10C, 10AE, 10W, and 11E; (147) 10A, 10B, 10C, 10AE, 10V, 10AH, and 11E; (148) 10I, 10R, 10AD, 10AH, and 11E; (149) 10E, 10F, 10R, 10AD, 10AH, and 11E; (150) 10E, 10Q, 10Z, 10AD, 10AH, and 11E; (151) 10E, 10Q, 10AC, 10AG, 10AH, and 11E; (152) 10P, 10N, 10AD, 10AH, and 11E; (153) 10P, 10Y, 10Z, 10AD, 10AH, and 11E; (154) 10P, 10Y, 10AC, 10AG, 10AH, and 11E; (155) 10P, 10AB, 10V, 10AH, and 11E; (156) 10P, 10AB, 10AF, 10AG, 10AH, and 11E; (157) 10P, 10AB, 10W, and 11E; (158) 1T, 10AS, 10I, 10R, 10AD, 10AH, and 11E; (159) 1T, 10AS, 10E, 10F, 10R, 10AD, 10AH, and 11E; (160) 1T, 10AS, 10E, 10Q, 10Z, 10AD, 10AH, and 11E; (161) 1T, 10AS, 10E, 10Q, 10AC, 10AG, 10AH, and 11E; (162) 1T, 10AS, 10P, 10N, 10AD, 10AH, and 11E; (163) 1T, 10AS, 10P, 10Y, 10Z, 10AD, 10AH, and 11E; (164) 1T, 10AS, 10P, 10Y, 10AC, 10AG, 10AH, and 11E; (165) 1T, 10AS, 10P, 10AB, 10V, 10AH, and 11E; (166) 1T, 10AS, 10P, 10AB, 10AF, 10AG, 10AH, and 11E; (167) 1T, 10AS, 10P, 10AB, 10W, and 11E; (168) 10AT, 10I, 10R, 10AD, 10AH, and 11E; (169) 10AT, 10E, 10F, 10R, 10AD, 10AH, and 11E; (170) 10AT, 10E, 10Q, 10Z, 10AD, 10AH, and 11E; (171) 10AT, 10E, 10Q, 10AC, 10AG, 10AH, and 11E; (172) 10AT, 10P, 10N, 10AD, 10AH, and 11E; (173) 10AT, 10P, 10Y, 10Z, 10AD, 10AH, and 11E; (174) 10AT, 10P, 10Y, 10AC, 10AG, 10AH, and 11E; (175) 10AT, 10P, 10AB, 10V, 10AH, and 11E; (176) 10AT, 10P, 10AB, 10AF, 10AG, 10AH, and 11E; (177) 10AT, 10P, 10AB, 10W, and 11E; (178) 10AU, 10AF, 10AG, 10AH, and 11E; (179) 10AU, 10W, and 11E; (180) 10AU, 10V, 10AH, and 11E; (181) 12A, 12B, 12C, 12D, 12E, 12F, 12G, 12H, and 121; (182) 12A, 12K, 12M, 12N, 12E, 12F, 12G, 12H, and 121; (183) 12A, 12K, 12L, 12D, 12E, 12F, 12G, 12H, and 121; (184) 12A, 12O, 12N, 12E, 12F, 12G, 12H, and 121; (185) 12A, 12B, 12J, 12E, 12F, 12G, 12H, and 121; (186) 10A, 10D, 10E, 10F, 10G, 10S, 15A, 15B, 15C, and 15G; (187) 10A, 10D, 10I, 10G, 10S, 15A, 15B, 15C, and 15G; (188) 10A, 10D, 10K, 10S, 15A, 15B, 15C, and 15G; (189) 10A, 10H, 10F, 10G, 10S, 15A, 15B, 15C, and 15G; (190) 10A, 10J, 10G, 10S, 15A, 15B, 15C, and 15G; (191) 10A, 10J, 10R, 10AA, 15A, 15B, 15C, and 15G; (192) 10A, 10H, 10F, 10R, 10AA, 15A, 15B, 15C, and 15G; (193) 10A, 10H, 10Q, 10Z, 10AA, 15A, 15B, 15C, and 15G; (194) 10A, 10D, 10I, 10R, 10AA, 15A, 15B, 15C, and 15G; (195) 10A, 10D, 10E, 10F, 10R, 10AA, 15A, 15B, 15C, and 15G; (196) 10A, 10D, 10E, 10Q, 10Z, 10AA, 15A, 15B, 15C, and 15G; (197) 10A, 10D, 10P, 10N, 10AA, 15A, 15B, 15C, and 15G; (198) 10A, 10D, 10P, 10Y, 10Z, 10AA, 15A, 15B, 15C, and 15G; (199) 10A, 10B, 10M, 10AA, 15A, 15B, 15C, and 15G; (200) 10A, 10B, 10L, 10Z, 10AA, 15A, 15B, 15C, and 15G; (201) 10A, 10B, 10X, 10N, 10AA, 15A, 15B, 15C, and 15G; (202) 10A, 10B, 10X, 10Y, 10Z, 10AA, 15A, 15B, 15C, and 15G; (203) 10A, 10D, 10P, 10O, 15A, 15B, 15C, and 15G; (204) 10A, 10B, 10X, 10O, 15A, 15B, 15C, and 15G; (205) 10A, 10D, 10E, 10F, 10R, 10AA, 15A, 15B, 15C, and 15G; (206) 10A, 10D, 10E, 10F, 10G, 10S, 15A, 15B, 15C, and 15G; (207) 10A, 10B, 10C, 10AE, 10AB, 10Y, 10Z, 10AA, 15A, 15B, 15C, and 15G; (208) 10A, 10B, 10C, 10AE, 10AB, 10N, 10AA, 15A, 15B, 15C, and 15G; (209) 10A, 10B, 10C, 10AE, 10AB, 10O, 15A, 15B, 15C, and 15G; (210) 10AU, 10AB, 10Y, 10Z, 10AA, 15A, 15B, 15C, and 15G; (211) 10AU, 10AB, 10N, 10AA, 15A, 15B, 15C, and 15G; (212) 10AU, 10AB, 10O, 15A, 15B, 15C, and 15G; (213) 1T, 10AS, 10E, 10F, 10G, 10S, 15A, 15B, 15C, and 15G; (214) 1T, 10AS, 10I, 10G, 10S, 15A, 15B, 15C, and 15G; (215) 1T, 10AS, 10K, 10S, 15A, 15B, 15C, and 15G; (216) 1T, 10AS, 10I, 10R, 10AA, 15A, 15B, 15C, and 15G; (217) 1T, 10AS, 10E, 10F, 10R, 10AA, 15A, 15B, 15C, and 15G; (218) 1T, 10AS, 10E, 10Q, 10Z, 10AA, 15A, 15B, 15C, and 15G; (219) 1T, 10AS, 10P, 10N, 10AA, 15A, 15B, 15C, and 15G; (220) 1T, 10AS, 10P, 10Y, 10Z, 10AA, 15A, 15B, 15C, and 15G; (221) 1T, 10AS, 10P, 10O, 15A, 15B, 15C, and 15G; (222) 1T, 10AS, 10E, 10F, 10R, 10AA, 15A, 15B, 15C, and 15G; (223) 10AT, 10E, 10F, 10G, 10S, 15A, 15B, 15C, and 15G; (224) 10AT, 10I, 10G, 10S, 15A, 15B, 15C, and 15G; (225) 10AT, 10K, 10S, 15A, 15B, 15C, and 15G; (226) 10AT, 10I, 10R, 10AA, 15A, 15B, 15C, and 15G; (227) 10AT, 10E, 10F, 10R, 10AA, 15A, 15B, 15C, and 15G; (228) 10AT, 10E, 10Q, 10Z, 10AA, 15A, 15B, 15C, and 15G; (229) 10AT, 10P, 10N, 10AA, 15A, 15B, 15C, and 15G; (230) 10AT, 10P, 10Y, 10Z, 10AA, 15A, 15B, 15C, and 15G; (231) 10AT, 10P, 10O, 15A, 15B, 15C, and 15G; (232) 10AT, 10E, 10F, 10R, 10AA, 15A, 15B, 15C, and 15G; (233) 10A, 10D, 10E, 10F, 10G, 10S, 15D, and 15G; (234) 10A, 10D, 10I, 10G, 10S, 15D, and 15G; (235) 10A, 10D, 10K, 10S, 15D, and 15G; (236) 10A, 10H, 10F, 10G, 10S, 15D, and 15G; (237) 10A, 10J, 10G, 10S, 15D, and 15G; (238) 10A, 10J, 10R, 10AA, 15D, and 15G; (239) 10A, 10H, 10F, 10R, 10AA, 15D, and 15G; (240) 10A, 10H, 10Q, 10Z, 10AA, 15D, and 15G; (241) 10A, 10D, 10I, 10R, 10AA, 15D, and 15G; (242) 10A, 10D, 10E, 10F, 10R, 10AA, 15D, and 15G; (243) 10A, 10D, 10E, 10Q, 10Z, 10AA, 15D, and 15G; (244) 10A, 10D, 10P, 10N, 10AA, 15D, and 15G; (245) 10A, 10D, 10P, 10Y, 10Z, 10AA, 15D, and 15G; (246) 10A, 10B, 10M, 10AA, 15D, and 15G; (247) 10A, 10B, 10L, 10Z, 10AA, 15D, and 15G; (248) 10A, 10B, 10X, 10N, 10AA, 15D, and 15G; (249) 10A, 10B, 10X, 10Y, 10Z, 10AA, 15D, and 15G; (250) 10A, 10D, 10P, 10O, 15D, and 15G; (251) 10A, 10B, 10X, 10O, 15D, and 15G; (252) 10A, 10D, 10E, 10F, 10R, 10AA, 15D, and 15G; (253) 10A, 10D, 10E, 10F, 10G, 10S, 15D, and 15G; (254) 10A, 10B, 10C, 10AE, 10AB, 10Y, 10Z, 10AA, 15D, and 15G; (255) 10A, 10B, 10C, 10AE, 10AB, 10N, 10AA, 15D, and 15G; (256) 10A, 10B, 10C, 10AE, 10AB, 10O, 15D, and 15G; (257) 10AU, 10AB, 10Y, 10Z, 10AA, 15D, and 15G; (258) 10AU, 10AB, 10N, 10AA, 15D, and 15G; (259) 10AU, 10AB, 10O, 15D, and 15G; (260) 1T, 10AS, 10E, 10F, 10G, 10S, 15D, and 15G; (261) 1T, 10AS, 10I, 10G, 10S, 15D, and 15G; (262) 1T, 10AS, 10K, 10S, 15D, and 15G; (263) 1T, 10AS, 10I, 10R, 10AA, 15D, and 15G; (264) 1T, 10AS, 10E, 10F, 10R, 10AA, 15D, and 15G; (265) 1T, 10AS, 10E, 10Q, 10Z, 10AA, 15D, and 15G; (266) 1T, 10AS, 10P, 10N, 10AA, 15D, and 15G; (267) 1T, 10AS, 10P, 10Y, 10Z, 10AA, 15D, and 15G; (268) 1T, 10AS, 10P, 10O, 15D, and 15G; (269) 1T, 10AS, 10E, 10F, 10R, 10AA, 15D, and 15G; (270) 10AT, 10E, 10F, 10G, 10S, 15D, and 15G; (271) 10AT, 10I, 10G, 10S, 15D, and 15G; (272) 10AT, 10K, 10S, 15D, and 15G; (273) 10AT, 10I, 10R, 10AA, 15D, and 15G; (274) 10AT, 10E, 10F, 10R, 10AA, 15D, and 15G; (275) 10AT, 10E, 10Q, 10Z, 10AA, 15D, and 15G; (276) 10AT, 10P, 10N, 10AA, 15D, and 15G; (277) 10AT, 10P, 10Y, 10Z, 10AA, 15D, and 15G; (278) 10AT, 10P, 10O, 15D, and 15G; (279) 10AT, 10E, 10F, 10R, 10AA, 15D, and 15G; (280) 10A, 10D, 10E, 10F, 10G, 10S, 15E, 15C, and 15G; (281) 10A, 10D, 10I, 10G, 10S, 15E, 15C, and 15G; (282) 10A, 10D, 10K, 10S, 15E, 15C, and 15G; (283) 10A, 10H, 10F, 10G, 10S, 15E, 15C, and 15G; (284) 10A, 10J, 10G, 10S, 15E, 15C, and 15G; (285) 10A, 10J, 10R, 10AA, 15E, 15C, and 15G; (286) 10A, 10H, 10F, 10R, 10AA, 15E, 15C, and 15G; (287) 10A, 10H, 10Q, 10Z, 10AA, 15E, 15C, and 15G; (288) 10A, 10D, 10I, 10R, 10AA, 15E, 15C, and 15G; (289) 10A, 10D, 10E, 10F, 10R, 10AA, 15E, 15C, and 15G; (290) 10A, 10D, 10E, 10Q, 10Z, 10AA, 15E, 15C, and 15G; (291) 10A, 10D, 10P, 10N, 10AA, 15E, 15C, and 15G; (292) 10A, 10D, 10P, 10Y, 10Z, 10AA, 15E, 15C, and 15G; (293) 10A, 10B, 10M, 10AA, 15E, 15C, and 15G; (294) 10A, 10B, 10L, 10Z, 10AA, 15E, 15C, and 15G; (295) 10A, 10B, 10X, 10N, 10AA, 15E, 15C, and 15G; (296) 10A, 10B, 10X, 10Y, 10Z, 10AA, 15E, 15C, and 15G; (297) 10A, 10D, 10P, 10O, 15E, 15C, and 15G; (298) 10A, 10B, 10X, 10O, 15E, 15C, and 15G; (299) 10A, 10D, 10E, 10F, 10R, 10AA, 15E, 15C, and 15G; (300) 10A, 10D, 10E, 10F, 10G, 10S, 15E, 15C, and 15G; (301) 10A, 10B, 10C, 10AE, 10AB, 10Y, 10Z, 10AA, 15E, 15C, and 15G; (302) 10A, 10B, 10C, 10AE, 10AB, 10N, 10AA, 15E, 15C, and 15G; (303) 10A, 10B, 10C, 10AE, 10AB, 10O, 15E, 15C, and 15G; (304) 10AU, 10AB, 10Y, 10Z, 10AA, 15E, 15C, and 15G; (305) 10AU, 10AB, 10N, 10AA, 15E, 15C, and 15G; (306) 10AU, 10AB, 10O, 15E, 15C, and 15G; (307) 1T, 10AS, 10E, 10F, 10G, 10S, 15E, 15C, and 15G; (308) 1T, 10AS, 10I, 10G, 10S, 15E, 15C, and 15G; (309) 1T, 10AS, 10K, 10S, 15E, 15C, and 15G; (310) 1T, 10AS, 10I, 10R, 10AA, 15E, 15C, and 15G; (311) 1T, 10AS, 10E, 10F, 10R, 10AA, 15E, 15C, and 15G; (312) 1T, 10AS, 10E, 10Q, 10Z, 10AA, 15E, 15C, and 15G; (313) 1T, 10AS, 10P, 10N, 10AA, 15E, 15C, and 15G; (314) 1T, 10AS, 10P, 10Y, 10Z, 10AA, 15E, 15C, and 15G; (315) 1T, 10AS, 10P, 10O, 15E, 15C, and 15G; (316) 1T, 10AS, 10E, 10F, 10R, 10AA, 15E, 15C, and 15G; (317) 10AT, 10E, 10F, 10G, 10S, 15E, 15C, and 15G; (318) 10AT, 10I, 10G, 10S, 15E, 15C, and 15G; (319) 10AT, 10K, 10S, 15E, 15C, and 15G; (320) 10AT, 10I, 10R, 10AA, 15E, 15C, and 15G; (321) 10AT, 10E, 10F, 10R, 10AA, 15E, 15C, and 15G; (322) 10AT, 10E, 10Q, 10Z, 10AA, 15E, 15C, and 15G; (323) 10AT, 10P, 10N, 10AA, 15E, 15C, and 15G; (324) 10AT, 10P, 10Y, 10Z, 10AA, 15E, 15C, and 15G; (325) 10AT, 10P, 10O, 15E, 15C, and 15G; (326) 10AT, 10E, 10F, 10R, 10AA, 15E, 15C, and 15G; (327) 10A, 10D, 10E, 10F, 10G, 10S, 15A, 15F, and 15G; (328) 10A, 10D, 10L 10G, 10S, 15A, 15F, and 15G; (329) 10A, 10D, 10K, 10S, 15A, 15F, and 15G; (330) 10A, 10H, 10F, 10G, 10S, 15A, 15F, and 15G; (331) 10A, 10J, 10G, 10S, 15A, 15F, and 15G; (332) 10A, 10J, 10R, 10AA, 15A, 15F, and 15G; (333) 10A, 10H, 10F, 10R, 10AA, 15A, 15F, and 15G; (334) 10A, 10H, 10Q, 10Z, 10AA, 15A, 15F, and 15G; (335) 10A, 10D, 10I, 10R, 10AA, 15A, 15F, and 15G; (336) 10A, 10D, 10E, 10F, 10R, 10AA, 15A, 15F, and 15G; (337) 10A, 10D, 10E, 10Q, 10Z, 10AA, 15A, 15F, and 15G; (338) 10A, 10D, 10P, 10N, 10AA, 15A, 15F, and 15G; (339) 10A, 10D, 10P, 10Y, 10Z, 10AA, 15A, 15F, and 15G; (340) 10A, 10B, 10M, 10AA, 15A, 15F, and 15G; (341) 10A, 10B, 10L, 10Z, 10AA, 15A, 15F, and 15G; (342) 10A, 10B, 10X, 10N, 10AA, 15A, 15F, and 15G; (343) 10A, 10B, 10X, 10Y, 10Z, 10AA, 15A, 15F, and 15G; (344) 10A, 10D, 10P, 10O, 15A, 15F, and 15G; (345) 10A, 10B, 10X, 10O, 15A, 15F, and 15G; (346) 10A, 10D, 10E, 10F, 10R, 10AA, 15A, 15F, and 15G; (347) 10A, 10D, 10E, 10F, 10G, 10S, 15A, 15F, and 15G; (348) 10A, 10B, 10C, 10AE, 10AB, 10Y, 10Z, 10AA, 15A, 15F, and 15G; (349) 10A, 10B, 10C, 10AE, 10AB, 10N, 10AA, 15A, 15F, and 15G; (350) 10A, 10B, 10C, 10AE, 10AB, 10O, 15A, 15F, and 15G; (351) 10AU, 10AB, 10Y, 10Z, 10AA, 15A, 15F, and 15G; (352) 10AU, 10AB, 10N, 10AA, 15A, 15F, and 15G; (353) 10AU, 10AB, 10O, 15A, 15F, and 15G; (354) 1T, 10AS, 10E, 10F, 10G, 10S, 15A, 15F, and 15G; (355) 1T, 10AS, 10I, 10G, 10S, 15A, 15F, and 15G; (356) 1T, 10AS, 10K, 10S, 15A, 15F, and 15G; (357) 1T, 10AS, 10I, 10R, 10AA, 15A, 15F, and 15G; (358) 1T, 10AS, 10E, 10F, 10R, 10AA, 15A, 15F, and 15G; (359) 1T, 10AS, 10E, 10Q, 10Z, 10AA, 15A, 15F, and 15G; (360) 1T, 10AS, 10P, 10N, 10AA, 15A, 15F, and 15G; (361) 1T, 10AS, 10P, 10Y, 10Z, 10AA, 15A, 15F, and 15G; (362) 1T, 10AS, 10P, 10O, 15A, 15F, and 15G; (363) 1T, 10AS, 10E, 10F, 10R, 10AA, 15A, 15F, and 15G; (364) 10AT, 10E, 10F, 10G, 10S, 15A, 15F, and 15G; (365) 10AT, 10I, 10G, 10S, 15A, 15F, and 15G; (366) 10AT, 10K, 10S, 15A, 15F, and 15G; (367) 10AT, 10I, 10R, 10AA, 15A, 15F, and 15G; (368) 10AT, 10E, 10F, 10R, 10AA, 15A, 15F, and 15G; (369) 10AT, 10E, 10Q, 10Z, 10AA, 15A, 15F, and 15G; (370) 10AT, 10P, 10N, 10AA, 15A, 15F, and 15G; (371) 10AT, 10P, 10Y, 10Z, 10AA, 15A, 15F, and 15G; (372) 10AT, 10P, 10O, 15A, 15F, and 15G; (373) 10AT, 10E, 10F, 10R, 10AA, 15A, 15F, and 15G; (374) 14A, 14B, 14C, 14D, 14E, 13A, and 13B; (375) 16A, 16B, 16C, 16D, and 16E; (376) 17A, 17B, 17C, 17D, and 17G; (377) 17A, 17E, 17F, 17D, and 17G; (378) 17A, 17B, 17C, 17H, 17I, 17J, and 17G; (379) 18A, 18B, 18C, 18D, 18E, and 18F; (380) 13A and 13B; and (381) 7A, 17E, 17F, 17H, 17I, 17J, and 17G,
wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10T is a crotonyl-ACP thioesterase, wherein 10U is a crotonyl-ACP reductase (aldehyde forming), wherein 10V is a crotonyl-CoA reductase (aldehyde forming), wherein 10W is a crotonyl-CoA (alcohol forming), wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AC is a 3-hydroxybutyrate dehydratase, wherein LOAD is a 3-hydroxybutyraldehyde dehydratase, wherein 10AE is a crotonyl-CoA:ACP transferase, wherein LOAF is a crotonyl-CoA hydrolase, transferase or synthetase, wherein 10AG is a crotonate reductase, wherein 10AH is a crotonaldehyde reductase, wherein 10AS is an acetoacetyl-CoA synthase, wherein 10AT is an acetyl-CoA:acetyl-CoA acyltransferase, wherein 10AU is a 4-hydroxybutyryl-CoA dehydratase, wherein 11A is a crotyl alcohol kinase, wherein 11B is a 2-butenyl-4-phosphate kinase, wherein 11C is a butadiene synthase, wherein 11D is a crotyl alcohol diphosphokinase, wherein 11E is a crotyl alcohol dehydratase, wherein 12A is a malonyl-CoA:acetyl-CoA acyltransferase, wherein 12B is a 3-oxoglutaryl-CoA reductase (ketone-reducing), wherein 12C is a 3-hydroxyglutaryl-CoA reductase (aldehyde forming), wherein 12D is a 3-hydroxy-5-oxopentanoate reductase, wherein 12E is a 3,5-dihydroxypentanoate kinase, wherein 12F is a 3-hydroxy-5-phosphonatooxypentanoate kinase, wherein 12G is a 3-hydroxy-5-[hydroxy(phosphonooxy)phosphoryl]oxy pentanoate decarboxylase, wherein 12H is a butenyl 4-diphosphate isomerase, wherein 12I is a butadiene synthase, wherein 12J is a 3-hydroxyglutaryl-CoA reductase (alcohol forming), wherein 12K is a 3-oxoglutaryl-CoA reductase (aldehyde forming), wherein 12L is a 3,5-dioxopentanoate reductase (ketone reducing), wherein 12M is a 3,5-dioxopentanoate reductase (aldehyde reducing), wherein 12N is a 5-hydroxy-3-oxopentanoate reductase, wherein 12O is a 3-oxo-glutaryl-CoA reductase (CoA reducing and alcohol forming), wherein 13A is a 2-butanol desaturase, wherein 13B is a 3-buten-2-ol dehydratase, wherein 14A is an acetolactate synthase, wherein 14B is an acetolactate decarboxylase, wherein 14C is a butanediol dehydrogenase, wherein 14D is a butanediol dehydratase, wherein 14E is a butanol dehydrogenase, wherein 15A is a 1,3-butanediol kinase, wherein 15B is a 3-hydroxybutyrylphosphate kinase, 15C is a 3-hydroxybutyryldiphosphate lyase, wherein 15D is a 1,3-butanediol diphosphokinase, wherein 15E is a 1,3-butanediol dehydratase, wherein 15F is a 3-hydroxybutyrylphosphate lyase, wherein 15G is a 3-buten-2-ol dehydratase, wherein 16A is a 3-oxopent-4-enoyl-CoA thiolase, wherein 16B is a 3-oxopent-4-enoyl-CoA hydrolase, synthetase or transferase, wherein 16C is a 3-oxopent-4-enoate decarboxylase or spontaneous, wherein 16D is a 3-buten-2-one reductase, wherein 16E is a 3-buten-2-ol dehydratase, wherein 17A is a 3-oxo-4-hydroxypentanoyl-CoA thiolase, wherein 17B is a 3-oxo-4-hydroxypentanoyl-CoA transferase, synthetase or hydrolase, wherein 17C is a 3-oxo-4-hydroxypentanoate reductase, wherein 17D is a 3,4-dihydroxypentanoate decarboxylase, wherein 17E is a 3-oxo-4-hydroxypentanoyl-CoA reductase, wherein 17F is a 3,4-dihydroxypentanoyl-CoA transferase, synthetase or hydrolase, wherein 17G is a 3-buten-2-ol dehydratase, wherein 17H is a 3,4-dihydroxypentanoate dehydratase, wherein 17I is a 4-oxopentanoate reductase, wherein 17J is a 4-hyd4-oxoperoxypentanoate decarboxylase, wherein 18A is a 3-oxoadipyl-CoA thiolase, wherein 18B is a 3-oxoadipyl-CoA transferase, synthetase or hydrolase, wherein 18C is a 3-oxoadipate decarboxylase or spontaneous, wherein 18D is a 4-oxopentanoate reductase, wherein 18E is a 4-hydroxypentanoate decarboxylase, wherein 18F is a 3-buten-2-ol dehydratase.

29-31. (canceled)

32. A method for producing butadiene comprising culturing the non-naturally occurring microbial organism of claim 14 under conditions and for a sufficient period of time to produce butadiene.

33-34. (canceled)

35. Bioderived butadiene produced according to the method of claim 32.

36-45. (canceled)

46. The non-naturally occurring microbial organism of claim 1, wherein said organism further comprises a crotyl alcohol pathway and comprising at least one exogenous nucleic acid encoding a crotyl alcohol pathway enzyme expressed in a sufficient amount to produce crotyl alcohol, wherein said crotyl alcohol pathway comprises a pathway selected from:
(1) 10A, 10J, 10R, 10AD, and 10AH; (2) 10A, 10H, 10F, 10R, 10AD, and 10AH; (3) 10A, 10H, 10Q, 10Z, 10AD, and 10AH; (4) 10A, 10H, 10Q, 10AC, 10AG, and 10AH; (5) 10A, 10D, 10I, 10R, 10AD, and 10AH; (6) 10A, 10D, 10E, 10F, 10R, 10AD, and 10AH; (7) 10A, 10D, 10E, 10Q, 10Z, 10AD, and 10AH; (8) 10A, 10D, 10E, 10Q, 10AC, 10AG, and 10AH; (9) 10A, 10D, 10P, 10N, 10AD, and 10AH; (10) 10A, 10D, 10P, 10Y, 10Z, 10AD, and 10AH; (11) 10A, 10D, 10P, 10Y, 10AC, 10AG, and 10AH; (12) 10A, 10D, 10P, 10AB, 10V, and 10AH; (13) 10A, 10D, 10P, 10AB, LOAF, 10AG, and 10AH; (14) 10A, 10B, 10M, 10AD, and 10AH; (15) 10A, 10B, 10L, 10Z, 10AD, and 10AH; (16) 10A, 10B, 10L, 10AC, 10AG, and 10AH; (17) 10A, 10B, 10X, 10Y, 10Z, 10AD, and 10AH; (18) 10A, 10B, 10X, 10Y, 10AC, 10AG, and 10AH; (19) 10A, 10B, 10X, 10AB, 10V, and 10AH; (20) 10A, 10B, 10X, 10AB, LOAF, 10AG, and 10AH; (21) 10A, 10B, 10C, 10U, and 10AH; (22) 10A, 10B, 10C, 10T, 10AG, and 10AH; (23) 10A, 10B, 10C, 10AE, 10AF, 10AG, and 10AH; (24) 10A, 10D, 10P, 10AB, and 10W; (25) 10A, 10B, 10X, 10AB, and 10W; (26) 10A, 10B, 10C, 10AE, and 10W; (27) 10A, 10B, 10C, 10AE, 10V, and 10AH; (28) 101, 10R, 10AD, and 10AH; (29) 10E, 10F, 10R, 10AD, and 10AH; (30) 10E, 10Q, 10Z, 10AD, and 10AH; (31) 10E, 10Q, 10AC, 10AG, and 10AH; (32) 10P, 10N, 10AD, and 10AH; (33) 10P, 10Y, 10Z, 10AD, and 10AH; (34) 10P, 10Y, 10AC, 10AG, and 10AH; (35) 10P, 10AB, 10V, and 10AH; (36) 10P, 10AB, 10AF, 10AG, and 10AH; (37) 10P, 10AB, and 10W; (38) 1T, 10AS, 10I, 10R, 10AD, and 10AH; (39) 1T, 10AS, 10E, 10F, 10R, 10AD, and 10AH; (40) 1T, 10AS, 10E, 10Q, 10Z, 10AD, and 10AH; (41) 1T, 10AS, 10E, 10Q, 10AC, 10AG, and 10AH; (42) 1T, 10AS, 10P, 10N, 10AD, and 10AH; (43) 1T, 10AS, 10P, 10Y, 10Z, 10AD, and 10AH; (44) 1T, 10AS, 10P, 10Y, 10AC, 10AG, and 10AH; (45) 1T, 10AS, 10P, 10AB, 10V, and 10AH; (46) 1T, 10AS, 10P, 10AB, 10AF, 10AG, and 10AH; (47) 1T, 10AS, 10P, 10AB, and 10W; (48) 10AT, 10I, 10R, 10AD, and 10AH; (49) 10AT, 10E, 10F, 10R, 10AD, and 10AH; (50) 10AT, 10E, 10Q, 10Z, 10AD, and 10AH; (51) 10AT, 10E, 10Q, 10AC, 10AG, and 10AH; (52) 10AT, 10P, 10N, 10AD, and 10AH; (53) 10AT, 10P, 10Y, 10Z, 10AD, and 10AH; (54) 10AT, 10P, 10Y, 10AC, 10AG, and 10AH; (55) 10AT, 10P, 10AB, 10V, and 10AH; (56) 10AT, 10P, 10AB, 10AF, 10AG, and 10AH; (57) 10AT, 10P, 10AB, and 10W; (58) 10AU, 10AF, 10AG, and 10AH; (59) 10AU, and 10W; and (60) 10AU, 10V, and 10AH,
wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 1 OH is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10T is a crotonyl-ACP thioesterase, wherein 10U is a crotonyl-ACP reductase (aldehyde forming), wherein 10V is a crotonyl-CoA reductase (aldehyde forming), wherein 10W is a crotonyl-CoA (alcohol forming), wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AB is a 3-hydroxybutyryl-CoA dehydratase, wherein 10AC is a 3-hydroxybutyrate dehydratase, wherein LOAD is a 3-hydroxybutyraldehyde dehydratase, wherein 10AE is a crotonyl-CoA:ACP transferase, wherein LOAF is a crotonyl-CoA hydrolase, transferase or synthetase, wherein 10AG is a crotonate reductase, wherein 10AH is a crotonaldehyde reductase, wherein 10AS is an acetoacetyl-CoA synthase, wherein 10AT is an acetyl-CoA:acetyl-CoA acyltransferase, wherein 10AU is a 4-hydroxybutyryl-CoA dehydratase.

47-49. (canceled)

50. A method for producing crotyl alcohol comprising culturing the non-naturally occurring microbial organism of claim 46 under conditions and for a sufficient period of time to produce crotyl alcohol.

51-52. (canceled)

53. Bioderived crotyl alcohol produced according to the method of claim 50.

54-66. (canceled)

67. The non-naturally occurring microbial organism of claim 1, wherein said organism further comprises a 1,3-butanediol pathway and comprising at least one exogenous nucleic acid encoding a 1,3-butanediol pathway enzyme expressed in a sufficient amount to produce 1,3-butanediol, wherein said 1,3-butanediol pathway comprises a pathway selected from:
(1) 10A, 10D, 10E, 10F, 10G, and 10S; (2) 10A, 10D, 10I, 10G, and 10S; (3) 10A, 10D, 10K, and 10S; (4) 10A, 10H, 10F, 10G, and 10S; (5) 10A, 10J, 10G, and 10S; (6) 10A, 10J, 10R, and 10AA; (7) 10A, 10H, 10F, 10R, and 10AA; (8) 10A, 10H, 10Q, 10Z, and 10AA; (9) 10A, 10D, 10I, 10R, and 10AA; (10) 10A, 10D, 10E, 10F, 10R, and 10AA; (11) 10A, 10D, 10E, 10Q, 10Z, and 10AA; (12) 10A, 10D, 10P, 10N, and 10AA; (13) 10A, 10D, 10P, 10Y, 10Z, and 10AA; (14) 10A, 10B, 10M, and 10AA; (15) 10A, 10B, 10L, 10Z, and 10AA; (16) 10A, 10B, 10X, 10N, and 10AA; (17) 10A, 10B, 10X, 10Y, 10Z, and 10AA; (18) 10A, 10D, 10P, and 10O; (19) 10A, 10B, 10X, and 10O; (20) 10A, 10D, 10E, 10F, 10R, and 10AA; (21) 10A, 10D, 10E, 10F, 10G, and 10S; (22) 10A, 10B, 10C, 10AE, 10AB, 10Y, 10Z, and 10AA; (23) 10A, 10B, 10C, 10AE, 10AB, 10N, and 10AA; (24) 10A, 10B, 10C, 10AE, 10AB, and 10O; (25) 10AU, 10AB, 10Y, 10Z, and 10AA; (26) 10AU, 10AB, 10N, and 10AA; (27) 10AU, 10AB, and 10O; (28) 1T, 10AS, 10E, 10F, 10G, and 10S; (29) 1T, 10AS, 10I, 10G, and 10S; (30) 1T, 10AS, 10K, and 10S; (31) 1T, 10AS, 10I, 10R, and 10AA; (32) 1T, 10AS, 10E, 10F, 10R, and 10AA; (33) 1T, 10AS, 10E, 10Q, 10Z, and 10AA; (34) 1T, 10AS, 10P, 10N, and 10AA; (35) 1T, 10AS, 10P, 10Y, 10Z, and 10AA; (36) 1T, 10AS, 10P, and 10O; (37) 1T, 10AS, 10E, 10F, 10R, and 10AA; (38) 10AT, 10E, 10F, 10G, and 105; (39) 10AT, 10I, 10G, and 105; (40) 10AT, 10K, and 105; (41) 10AT, 10I, 10R, and 10AA; (42) 10AT, 10E, 10F, 10R, and 10AA; (43) 10AT, 10E, 10Q, 10Z, and 10AA; (44) 10AT, 10P, 10N, and 10AA; (45) 10AT, 10P, 10Y, 10Z, and 10AA; (46) 10AT, 10P, and 10O; and (47) 10AT, 10E, 10F, 10R, and 10AA,
wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AE is a crotonyl-CoA:ACP transferase, wherein 10AS is an acetoacetyl-CoA synthase, wherein 10AT is an acetyl-CoA:acetyl-CoA acyltransferase, wherein 10AU is a 4-hydroxybutyryl-CoA dehydratase.

68-70. (canceled)

71. A method for producing 1,3-butanediol comprising culturing the non-naturally occurring microbial organism of claim 67 under conditions and for a sufficient period of time to produce 1,3-butanediol.

72-73. (canceled)

74. Bioderived 1,3-butanediol produced according to the method of claim 71.

75-87. (canceled)

88. A non-naturally occurring microbial organism having a 3-buten-2-ol pathway and comprising at least one exogenous nucleic acid encoding a 3-buten-2-ol pathway enzyme expressed in a sufficient amount to produce 3-buten-2-ol, wherein said 3-buten-2-ol pathway comprises a pathway selected from:
(1) 10A, 10D, 10E, 10F, 10G, 10S, 15A, 15B, and 15C; (2) 10A, 10D, 10I, 10G, 10S, 15A, 15B, and 15C; (3) 10A, 10D, 10K, 10S, 15A, 15B, and 15C; (4) 10A, 10H, 10F, 10G, 10S, 15A, 15B, and 15C; (5) 10A, 10J, 10G, 10S, 15A, 15B, and 15C; (6) 10A, 10J, 10R, 10AA, 15A, 15B, and 15C; (7) 10A, 10H, 10F, 10R, 10AA, 15A, 15B, and 15C; (8) 10A, 10H, 10Q, 10Z, 10AA, 15A, 15B, and 15C; (9) 10A, 10D, 10I, 10R, 10AA, 15A, 15B, and 15C; (10) 10A, 10D, 10E, 10F, 10R, 10AA, 15A, 15B, and 15C; (11) 10A, 10D, 10E, 10Q, 10Z, 10AA, 15A, 15B, and 15C; (12) 10A, 10D, 10P, 10N, 10AA, 15A, 15B, and 15C; (13) 10A, 10D, 10P, 10Y, 10Z, 10AA, 15A, 15B, and 15C; (14) 10A, 10B, 10M, 10AA, 15A, 15B, and 15C; (15) 10A, 10B, 10L, 10Z, 10AA, 15A, 15B, and 15C; (16) 10A, 10B, 10X, 10N, 10AA, 15A, 15B, and 15C; (17) 10A, 10B, 10X, 10Y, 10Z, 10AA, 15A, 15B, and 15C; (18) 10A, 10D, 10P, 10O, 15A, 15B, and 15C; (19) 10A, 10B, 10X, 10O, 15A, 15B, and 15C; (20) 10A, 10D, 10E, 10F, 10R, 10AA, 15A, 15B, and 15C; (21) 10A, 10D, 10E, 10F, 10G, 10S, 15A, 15B, and 15C; (22) 10A, 10B, 10C, 10AE, 10AB, 10Y, 10Z, 10AA, 15A, 15B, and 15C; (23) 10A, 10B, 10C, 10AE, 10AB, 10N, 10AA, 15A, 15B, and 15C; (24) 10A, 10B, 10C, 10AE, 10AB, 10O, 15A, 15B, and 15C; (25) 10AU, 10AB, 10Y, 10Z, 10AA, 15A, 15B, and 15C; (26) 10AU, 10AB, 10N, 10AA, 15A, 15B, and 15C; (27) 10AU, 10AB, 10O, 15A, 15B, and 15C; (28) 1T, 10AS, 10E, 10F, 10G, 10S, 15A, 15B, and 15C; (29) 1T, 10AS, 10I, 10G, 10S, 15A, 15B, and 15C; (30) 1T, 10AS, 10K, 10S, 15A, 15B, and 15C; (31) 1T, 10AS, 10I, 10R, 10AA, 15A, 15B, and 15C; (32) 1T, 10AS, 10E, 10F, 10R, 10AA, 15A, 15B, and 15C; (33) 1T, 10AS, 10E, 10Q, 10Z, 10AA, 15A, 15B, and 15C; (34) 1T, 10AS, 10P, 10N, 10AA, 15A, 15B, and 15C; (35) 1T, 10AS, 10P, 10Y, 10Z, 10AA, 15A, 15B, and 15C; (36) 1T, 10AS, 10P, 10O, 15A, 15B, and 15C; (37) 1T, 10AS, 10E, 10F, 10R, 10AA, 15A, 15B, and 15C; (38) 10AT, 10E, 10F, 10G, 10S, 15A, 15B, and 15C; (39) 10AT, 10I, 10G, 10S, 15A, 15B, and 15C; (40) 10AT, 10K, 10S, 15A, 15B, and 15C; (41) 10AT, 10I, 10R, 10AA, 15A, 15B, and 15C; (42) 10AT, 10E, 10F, 10R, 10AA, 15A, 15B, and 15C; (43) 10AT, 10E, 10Q, 10Z, 10AA, 15A, 15B, and 15C; (44) 10AT, 10P, 10N, 10AA, 15A, 15B, and 15C; (45) 10AT, 10P, 10Y, 10Z, 10AA, 15A, 15B, and 15C; (46) 10AT, 10P, 10O, 15A, 15B, and 15C; (47) 10AT, 10E, 10F, 10R, 10AA, 15A, 15B, and 15C; (48) 10A, 10D, 10E, 10F, 10G, 10S, and 15D; (49) 10A, 10D, 10I, 10G, 10S, and 15D; (50) 10A, 10D, 10K, 10S, and 15D; (51) 10A, 10H, 10F, 10G, 10S, and 15D; (52) 10A, 10J, 10G, 10S, and 15D; (53) 10A, 10J, 10R, 10AA, and 15D; (54) 10A, 10H, 10F, 10R, 10AA, and 15D; (55) 10A, 10H, 10Q, 10Z, 10AA, and 15D; (56) 10A, 10D, 10I, 10R, 10AA, and 15D; (57) 10A, 10D, 10E, 10F, 10R, 10AA, and 15D; (58) 10A, 10D, 10E, 10Q, 10Z, 10AA, and 15D; (59) 10A, 10D, 10P, 10N, 10AA, and 15D; (60) 10A, 10D, 10P, 10Y, 10Z, 10AA, and 15D; (61) 10A, 10B, 10M, 10AA, and 15D; (62) 10A, 10B, 10L, 10Z, 10AA, and 15D; (63) 10A, 10B, 10X, 10N, 10AA, and 15D; (64) 10A, 10B, 10X, 10Y, 10Z, 10AA, and 15D; (65) 10A, 10D, 10P, 10O, and 15D; (66) 10A, 10B, 10X, 10O, and 15D; (67) 10A, 10D, 10E, 10F, 10R, 10AA, and 15D; (68) 10A, 10D, 10E, 10F, 10G, 10S, and 15D; (69) 10A, 10B, 10C, 10AE, 10AB, 10Y, 10Z, 10AA, and 15D; (70) 10A, 10B, 10C, 10AE, 10AB, 10N, 10AA, and 15D; (71) 10A, 10B, 10C, 10AE, 10AB, 10O, and 15D; (72) 10AU, 10AB, 10Y, 10Z, 10AA, and 15D; (73) 10AU, 10AB, 10N, 10AA, and 15D; (74) 10AU, 10AB, 10O, and 15D; (75) 1T, 10AS, 10E, 10F, 10G, 10S, and 15D; (76) 1T, 10AS, 10I, 10G, 10S, and 15D; (77) 1T, 10AS, 10K, 10S, and 15D; (78) 1T, 10AS, 10I, 10R, 10AA, and 15D; (79) 1T, 10AS, 10E, 10F, 10R, 10AA, and 15D; (80) 1T, 10AS, 10E, 10Q, 10Z, 10AA, and 15D; (81) 1T, 10AS, 10P, 10N, 10AA, and 15D; (82) 1T, 10AS, 10P, 10Y, 10Z, 10AA, and 15D; (83) 1T, 10AS, 10P, 10O, and 15D; (84) 1T, 10AS, 10E, 10F, 10R, 10AA, and 15D; (85) 10AT, 10E, 10F, 10G, 10S, and 15D; (86) 10AT, 10I, 10G, 10S, and 15D; (87) 10AT, 10K, 10S, and 15D; (88) 10AT, 10I, 10R, 10AA, and 15D; (89) 10AT, 10E, 10F, 10R, 10AA, and 15D; (90) 10AT, 10E, 10Q, 10Z, 10AA, and 15D; (91) 10AT, 10P, 10N, 10AA, and 15D; (92) 10AT, 10P, 10Y, 10Z, 10AA, and 15D; (93) 10AT, 10P, 10O, and 15D; (94) 10AT, 10E, 10F, 10R, 10AA, and 15D; (95) 10A, 10D, 10E, 10F, 10G, 10S, 15E, and 15C; (96) 10A, 10D, 10I, 10G, 10S, 15E, and 15C; (97) 10A, 10D, 10K, 10S, 15E, and 15C; (98) 10A, 10H, 10F, 10G, 10S, 15E, and 15C; (99) 10A, 10J, 10G, 10S, 15E, and 15C; (100) 10A, 10J, 10R, 10AA, 15E, and 15C; (101) 10A, 10H, 10F, 10R, 10AA, 15E, and 15C; (102) 10A, 10H, 10Q, 10Z, 10AA, 15E, and 15C; (103) 10A, 10D, 10I, 10R, 10AA, 15E, and 15C; (104) 10A, 10D, 10E, 10F, 10R, 10AA, 15E, and 15C; (105) 10A, 10D, 10E, 10Q, 10Z, 10AA, 15E, and 15C; (106) 10A, 10D, 10P, 10N, 10AA, 15E, and 15C; (107) 10A, 10D, 10P, 10Y, 10Z, 10AA, 15E, and 15C; (108) 10A, 10B, 10M, 10AA, 15E, and 15C; (109) 10A, 10B, 10L, 10Z, 10AA, 15E, and 15C; (110) 10A, 10B, 10X, 10N, 10AA, 15E, and 15C; (111) 10A, 10B, 10X, 10Y, 10Z, 10AA, 15E, and 15C; (112) 10A, 10D, 10P, 10O, 15E, and 15C; (113) 10A, 10B, 10X, 10O, 15E, and 15C; (114) 10A, 10D, 10E, 10F, 10R, 10AA, 15E, and 15C; (115) 10A, 10D, 10E, 10F, 10G, 10S, 15E, and 15C; (116) 10A, 10B, 10C, 10AE, 10AB, 10Y, 10Z, 10AA, 15E, and 15C; (117) 10A, 10B, 10C, 10AE, 10AB, 10N, 10AA, 15E, and 15C; (118) 10A, 10B, 10C, 10AE, 10AB, 10O, 15E, and 15C; (119) 10AU, 10AB, 10Y, 10Z, 10AA, 15E, and 15C; (120) 10AU, 10AB, 10N, 10AA, 15E, and 15C; (121) 10AU, 10AB, 10O, 15E, and 15C; (122) 1T, 10AS, 10E, 10F, 10G, 10S, 15E, and 15C; (123) 1T, 10AS, 10I, 10G, 10S, 15E, and 15C; (124) 1T, 10AS, 10K, 10S, 15E, and 15C; (125) 1T, 10AS, 10I, 10R, 10AA, 15E, and 15C; (126) 1T, 10AS, 10E, 10F, 10R, 10AA, 15E, and 15C; (127) 1T, 10AS, 10E, 10Q, 10Z, 10AA, 15E, and 15C; (128) 1T, 10AS, 10P, 10N, 10AA, 15E, and 15C; (129) 1T, 10AS, 10P, 10Y, 10Z, 10AA, 15E, and 15C; (130) 1T, 10AS, 10P, 10O, 15E, and 15C; (131) 1T, 10AS, 10E, 10F, 10R, 10AA, 15E, and 15C; (132) 10AT, 10E, 10F, 10G, 10S, 15E, and 15C; (133) 10AT, 10I, 10G, 10S, 15E, and 15C; (134) 10AT, 10K, 10S, 15E, and 15C; (135) 10AT, 10I, 10R, 10AA, 15E, and 15C; (136) 10AT, 10E, 10F, 10R, 10AA, 15E, and 15C; (137) 10AT, 10E, 10Q, 10Z, 10AA, 15E, and 15C; (138) 10AT, 10P, 10N, 10AA, 15E, and 15C; (139) 10AT, 10P, 10Y, 10Z, 10AA, 15E, and 15C; (140) 10AT, 10P, 10O, 15E, and 15C; (141) 10AT, 10E, 10F, 10R, 10AA, 15E, and 15C; (142) 10A, 10D, 10E, 10F, 10G, 10S, 15A, and 15F; (143) 10A, 10D, 10I, 10G, 10S, 15A, and 15F; (144) 10A, 10D, 10K, 10S, 15A, and 15F; (145) 10A, 10H, 10F, 10G, 10S, 15A, and 15F; (146) 10A, 10J, 10G, 10S, 15A, and 15F; (147) 10A, 10J, 10R, 10AA, 15A, and 15F; (148) 10A, 10H, 10F, 10R, 10AA, 15A, and 15F; (149) 10A, 10H, 10Q, 10Z, 10AA, 15A, and 15F; (150) 10A, 10D, 10I, 10R, 10AA, 15A, and 15F; (151) 10A, 10D, 10E, 10F, 10R, 10AA, 15A, and 15F; (152) 10A, 10D, 10E, 10Q, 10Z, 10AA, 15A, and 15F; (153) 10A, 10D, 10P, 10N, 10AA, 15A, and 15F; (154) 10A, 10D, 10P, 10Y, 10Z, 10AA, 15A, and 15F; (155) 10A, 10B, 10M, 10AA, 15A, and 15F; (156) 10A, 10B, 10L, 10Z, 10AA, 15A, and 15F; (157) 10A, 10B, 10X, 10N, 10AA, 15A, and 15F; (158) 10A, 10B, 10X, 10Y, 10Z, 10AA, 15A, and 15F; (159) 10A, 10D, 10P, 10O, 15A, and 15F; (160) 10A, 10B, 10X, 10O, 15A, and 15F; (161) 10A, 10D, 10E, 10F, 10R, 10AA, 15A, and 15F; (162) 10A, 10D, 10E, 10F, 10G, 10S, 15A, and 15F; (163) 10A, 10B, 10C, 10AE, 10AB, 10Y, 10Z, 10AA, 15A, and 15F; (164) 10A, 10B, 10C, 10AE, 10AB, 10N, 10AA, 15A, and 15F; (165) 10A, 10B, 10C, 10AE, 10AB, 10O, 15A, and 15F; (166) 10AU, 10AB, 10Y, 10Z, 10AA, 15A, and 15F; (167) 10AU, 10AB, 10N, 10AA, 15A, and 15F; (168) 10AU, 10AB, 10O, 15A, and 15F; (169) 1T, 10AS, 10E, 10F, 10G, 10S, 15A, and 15F; (170) 1T, 10AS, 10I, 10G, 10S, 15A, and 15F; (171) 1T, 10AS, 10K, 10S, 15A, and 15F; (172) 1T, 10AS, 10I, 10R, 10AA, 15A, and 15F; (173) 1T, 10AS, 10E, 10F, 10R, 10AA, 15A, and 15F; (174) 1T, 10AS, 10E, 10Q, 10Z, 10AA, 15A, and 15F; (175) 1T, 10AS, 10P, 10N, 10AA, 15A, and 15F; (176) 1T, 10AS, 10P, 10Y, 10Z, 10AA, 15A, and 15F; (177) 1T, 10AS, 10P, 10O, 15A, and 15F; (178) 1T, 10AS, 10E, 10F, 10R, 10AA, 15A, and 15F; (179) 10AT, 10E, 10F, 10G, 10S, 15A, and 15F; (180) 10AT, 10I, 10G, 10S, 15A, and 15F; (181) 10AT, 10K, 10S, 15A, and 15F; (182) 10AT, 10I, 10R, 10AA, 15A, and 15F; (183) 10AT, 10E, 10F, 10R, 10AA, 15A, and 15F; (184) 10AT, 10E, 10Q, 10Z, 10AA, 15A, and 15F; (185) 10AT, 10P, 10N, 10AA, 15A, and 15F; (186) 10AT, 10P, 10Y, 10Z, 10AA, 15A, and 15F; (187) 10AT, 10P, 10O, 15A, and 15F; (188) 10AT, 10E, 10F, 10R, 10AA, 15A, and 15F; (189) 14A, 14B, 14C, 14D, 14E, and 13A; (190) 16A, 16B, 16C, and 16D; (191) 17A, 17B, 17C, and 17D; (192) 17A, 17E, 17F, and 17D; (193) 17A, 17B, 17C, 17H, 17I, and 17J; (194) 18A, 18B, 18C, 18D, and 18E; and (195) 17A, 17E, 17F, 17H, 17I, and 17J,
wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AE is a crotonyl-CoA:ACP transferase, wherein 10AS is an acetoacetyl-CoA synthase, wherein 10AT is an acetyl-CoA:acetyl-CoA acyltransferase, wherein 10AU is a 4-hydroxybutyryl-CoA dehydratase, wherein 13A is a 2-butanol desaturase, wherein 14A is an acetolactate synthase, wherein 14B is an acetolactate decarboxylase, wherein 14C is a butanediol dehydrogenase, wherein 14D is a butanediol dehydratase, wherein 14E is a butanol dehydrogenase, wherein 15A is a 1,3-butanediol kinase, wherein 15B is a 3-hydroxybutyrylphosphate kinase, 15C is a 3-hydroxybutyryldiphosphate lyase, wherein 15D is a 1,3-butanediol diphosphokinase, wherein 15E is a 1,3-butanediol dehydratase, wherein 15F is a 3-hydroxybutyrylphosphate lyase, wherein 16A is a 3-oxopent-4-enoyl-CoA thiolase, wherein 16B is a 3-oxopent-4-enoyl-CoA hydrolase, synthetase or transferase, wherein 16C is a 3-oxopent-4-enoate decarboxylase or spontaneous, wherein 16D is a 3-buten-2-one reductase, wherein 17A is a 3-oxo-4-hydroxypentanoyl-CoA thiolase, wherein 17B is a 3-oxo-4-hydroxypentanoyl-CoA transferase, synthetase or hydrolase, wherein 17C is a 3-oxo-4-hydroxypentanoate reductase, wherein 17D is a 3,4-dihydroxypentanoate decarboxylase, wherein 17E is a 3-oxo-4-hydroxypentanoyl-CoA reductase, wherein 17F is a 3,4-dihydroxypentanoyl-CoA transferase, synthetase or hydrolase, wherein 17H is a 3,4-dihydroxypentanoate dehydratase, wherein 17I is a 4-oxopentanoate reductase, wherein 17J is a 4-hyd4-oxoperoxypentanoate decarboxylase, wherein 18A is a 3-oxoadipyl-CoA thiolase, wherein 18B is a 3-oxoadipyl-CoA transferase, synthetase or hydrolase, wherein 18C is a 3-oxoadipate decarboxylase or spontaneous, wherein 18D is a 4-oxopentanoate reductase, wherein 18E is a 4-hydroxypentanoate decarboxylase.

89-90. (canceled)

91. The non-naturally occurring microbial organism of claim 88, wherein said microbial organism further comprises a formaldehyde fixation pathway comprising at least one exogenous nucleic acid encoding a formaldehyde fixation pathway enzyme expressed in a sufficient amount to produce pyruvate, wherein said formaldehyde fixation pathway comprises:
(1) 1B and 1C; or (2) 1D,
wherein 1B is a 3-hexulose-6-phosphate synthase, wherein 1C is a 6-phospho-3-hexuloisomerase, wherein 1D is a dihydroxyacetone synthase.

92-93. (canceled)

94. The non-naturally occurring microbial organism of claim 91, wherein said organism comprises at least one exogenous nucleic acid encoding a methanol metabolic pathway enzyme expressed in a sufficient amount to produce formaldehyde or produce or enhance the availability of reducing equivalents in the presence of methanol, wherein said methanol metabolic pathway comprises a pathway selected from:
(1) 3J; (2) 3A and 3B; (3) 3A, 3B and 3C; (4) 3J, 3K and 3C; (5) 3J, 3M, and 3N; (6) 3J and 3L; (7) 3A, 3B, 3C, 3D, and 3E; (8) 3A, 3B, 3C, 3D, and 3F; (9) 3J, 3K, 3C, 3D, and 3E; (10) 3J, 3K, 3C, 3D, and 3F; (11) 3J, 3M, 3N, and 3O; (12) 3A, 3B, 3C, 3D, 3E, and 3G; (13) 3A, 3B, 3C, 3D, 3F, and 3G; (14) 3J, 3K, 3C, 3D, 3E, and 3G; (15) 3J, 3K, 3C, 3D, 3F, and 3G; (16) 3J, 3M, 3N, 3O, and 3G; (17) 3A, 3B, 3C, 3D, 3E, and 3I; (18) 3A, 3B, 3C, 3D, 3F, and 3I; (19) 3J, 3K, 3C, 3D, 3E, and 3I; (20) 3J, 3K, 3C, 3D, 3F, and 3I; and (21) 3J, 3M, 3N, 3O, and 3I,
wherein 3A is a methanol methyltransferase, wherein 3B is a methylenetetrahydrofolate reductase, wherein 3C is a methylenetetrahydrofolate dehydrogenase, wherein 3D is a methenyltetrahydrofolate cyclohydrolase, wherein 3E is a formyltetrahydrofolate deformylase, wherein 3F is a formyltetrahydrofolate synthetase, wherein 3G is a formate hydrogen lyase, wherein 3H is a hydrogenase, wherein 3I is a formate dehydrogenase, wherein 3J is a methanol dehydrogenase, wherein 3K is a formaldehyde activating enzyme or spontaneous, wherein 3L is a formaldehyde dehydrogenase, wherein 3M is a S-(hydroxymethyl)glutathione synthase or spontaneous, wherein 3N is a glutathione-dependent formaldehyde dehydrogenase, wherein 3O is a S-formylglutathione hydrolase,

95-101. (canceled)

102. The non-naturally occurring microbial organism of claim 1, wherein said organism further comprises a 3-buten-2-ol pathway and comprising at least one exogenous nucleic acid encoding a 3-buten-2-ol pathway enzyme expressed in a sufficient amount to produce 3-buten-2-ol, wherein said 3-buten-2-ol pathway comprises a pathway selected from:
(1) 10A, 10D, 10E, 10F, 10G, 10S, 15A, 15B, and 15C; (2) 10A, 10D, 10I, 10G, 10S, 15A, 15B, and 15C; (3) 10A, 10D, 10K, 10S, 15A, 15B, and 15C; (4) 10A, 10H, 10F, 10G, 10S, 15A, 15B, and 15C; (5) 10A, 10J, 10G, 10S, 15A, 15B, and 15C; (6) 10A, 10J, 10R, 10AA, 15A, 15B, and 15C; (7) 10A, 10H, 10F, 10R, 10AA, 15A, 15B, and 15C; (8) 10A, 10H, 10Q, 10Z, 10AA, 15A, 15B, and 15C; (9) 10A, 10D, 10I, 10R, 10AA, 15A, 15B, and 15C; (10) 10A, 10D, 10E, 10F, 10R, 10AA, 15A, 15B, and 15C; (11) 10A, 10D, 10E, 10Q, 10Z, 10AA, 15A, 15B, and 15C; (12) 10A, 10D, 10P, 10N, 10AA, 15A, 15B, and 15C; (13) 10A, 10D, 10P, 10Y, 10Z, 10AA, 15A, 15B, and 15C; (14) 10A, 10B, 10M, 10AA, 15A, 15B, and 15C; (15) 10A, 10B, 10L, 10Z, 10AA, 15A, 15B, and 15C; (16) 10A, 10B, 10X, 10N, 10AA, 15A, 15B, and 15C; (17) 10A, 10B, 10X, 10Y, 10Z, 10AA, 15A, 15B, and 15C; (18) 10A, 10D, 10P, 10O, 15A, 15B, and 15C; (19) 10A, 10B, 10X, 10O, 15A, 15B, and 15C; (20) 10A, 10D, 10E, 10F, 10R, 10AA, 15A, 15B, and 15C; (21) 10A, 10D, 10E, 10F, 10G, 10S, 15A, 15B, and 15C; (22) 10A, 10B, 10C, 10AE, 10AB, 10Y, 10Z, 10AA, 15A, 15B, and 15C; (23) 10A, 10B, 10C, 10AE, 10AB, 10N, 10AA, 15A, 15B, and 15C; (24) 10A, 10B, 10C, 10AE, 10AB, 10O, 15A, 15B, and 15C; (25) 10AU, 10AB, 10Y, 10Z, 10AA, 15A, 15B, and 15C; (26) 10AU, 10AB, 10N, 10AA, 15A, 15B, and 15C; (27) 10AU, 10AB, 10O, 15A, 15B, and 15C; (28) 1T, 10AS, 10E, 10F, 10G, 10S, 15A, 15B, and 15C; (29) 1T, 10AS, 10I, 10G, 10S, 15A, 15B, and 15C; (30) 1T, 10AS, 10K, 10S, 15A, 15B, and 15C; (31) 1T, 10AS, 10I, 10R, 10AA, 15A, 15B, and 15C; (32) 1T, 10AS, 10E, 10F, 10R, 10AA, 15A, 15B, and 15C; (33) 1T, 10AS, 10E, 10Q, 10Z, 10AA, 15A, 15B, and 15C; (34) 1T, 10AS, 10P, 10N, 10AA, 15A, 15B, and 15C; (35) 1T, 10AS, 10P, 10Y, 10Z, 10AA, 15A, 15B, and 15C; (36) 1T, 10AS, 10P, 10O, 15A, 15B, and 15C; (37) 1T, 10AS, 10E, 10F, 10R, 10AA, 15A, 15B, and 15C; (38) 10AT, 10E, 10F, 10G, 10S, 15A, 15B, and 15C; (39) 10AT, 10I, 10G, 10S, 15A, 15B, and 15C; (40) 10AT, 10K, 10S, 15A, 15B, and 15C; (41) 10AT, 10I, 10R, 10AA, 15A, 15B, and 15C; (42) 10AT, 10E, 10F, 10R, 10AA, 15A, 15B, and 15C; (43) 10AT, 10E, 10Q, 10Z, 10AA, 15A, 15B, and 15C; (44) 10AT, 10P, 10N, 10AA, 15A, 15B, and 15C; (45) 10AT, 10P, 10Y, 10Z, 10AA, 15A, 15B, and 15C; (46) 10AT, 10P, 10O, 15A, 15B, and 15C; (47) 10AT, 10E, 10F, 10R, 10AA, 15A, 15B, and 15C; (48) 10A, 10D, 10E, 10F, 10G, 10S, and 15D; (49) 10A, 10D, 10I, 10G, 10S, and 15D; (50) 10A, 10D, 10K, 10S, and 15D; (51) 10A, 10H, 10F, 10G, 10S, and 15D; (52) 10A, 10J, 10G, 10S, and 15D; (53) 10A, 10J, 10R, 10AA, and 15D; (54) 10A, 10H, 10F, 10R, 10AA, and 15D; (55) 10A, 10H, 10Q, 10Z, 10AA, and 15D; (56) 10A, 10D, 10I, 10R, 10AA, and 15D; (57) 10A, 10D, 10E, 10F, 10R, 10AA, and 15D; (58) 10A, 10D, 10E, 10Q, 10Z, 10AA, and 15D; (59) 10A, 10D, 10P, 10N, 10AA, and 15D; (60) 10A, 10D, 10P, 10Y, 10Z, 10AA, and 15D; (61) 10A, 10B, 10M, 10AA, and 15D; (62) 10A, 10B, 10L, 10Z, 10AA, and 15D; (63) 10A, 10B, 10X, 10N, 10AA, and 15D; (64) 10A, 10B, 10X, 10Y, 10Z, 10AA, and 15D; (65) 10A, 10D, 10P, 10O, and 15D; (66) 10A, 10B, 10X, 10O, and 15D; (67) 10A, 10D, 10E, 10F, 10R, 10AA, and 15D; (68) 10A, 10D, 10E, 10F, 10G, 10S, and 15D; (69) 10A, 10B, 10C, 10AE, 10AB, 10Y, 10Z, 10AA, and 15D; (70) 10A, 10B, 10C, 10AE, 10AB, 10N, 10AA, and 15D; (71) 10A, 10B, 10C, 10AE, 10AB, 10O, and 15D; (72) 10AU, 10AB, 10Y, 10Z, 10AA, and 15D; (73) 10AU, 10AB, 10N, 10AA, and 15D; (74) 10AU, 10AB, 10O, and 15D; (75) 1T, 10AS, 10E, 10F, 10G, 10S, and 15D; (76) 1T, 10AS, 10I, 10G, 10S, and 15D; (77) 1T, 10AS, 10K, 10S, and 15D; (78) 1T, 10AS, 10I, 10R, 10AA, and 15D; (79) 1T, 10AS, 10E, 10F, 10R, 10AA, and 15D; (80) 1T, 10AS, 10E, 10Q, 10Z, 10AA, and 15D; (81) 1T, 10AS, 10P, 10N, 10AA, and 15D; (82) 1T, 10AS, 10P, 10Y, 10Z, 10AA, and 15D; (83) 1T, 10AS, 10P, 10O, and 15D; (84) 1T, 10AS, 10E, 10F, 10R, 10AA, and 15D; (85) 10AT, 10E, 10F, 10G, 10S, and 15D; (86) 10AT, 10I, 10G, 10S, and 15D; (87) 10AT, 10K, 10S, and 15D; (88) 10AT, 10I, 10R, 10AA, and 15D; (89) 10AT, 10E, 10F, 10R, 10AA, and 15D; (90) 10AT, 10E, 10Q, 10Z, 10AA, and 15D; (91) 10AT, 10P, 10N, 10AA, and 15D; (92) 10AT, 10P, 10Y, 10Z, 10AA, and 15D; (93) 10AT, 10P, 10O, and 15D; (94) 10AT, 10E, 10F, 10R, 10AA, and 15D; (95) 10A, 10D, 10E, 10F, 10G, 10S, 15E, and 15C; (96) 10A, 10D, 10I, 10G, 10S, 15E, and 15C; (97) 10A, 10D, 10K, 10S, 15E, and 15C; (98) 10A, 10H, 10F, 10G, 10S, 15E, and 15C; (99) 10A, 10J, 10G, 10S, 15E, and 15C; (100) 10A, 10J, 10R, 10AA, 15E, and 15C; (101) 10A, 10H, 10F, 10R, 10AA, 15E, and 15C; (102) 10A, 10H, 10Q, 10Z, 10AA, 15E, and 15C; (103) 10A, 10D, 10I, 10R, 10AA, 15E, and 15C; (104) 10A, 10D, 10E, 10F, 10R, 10AA, 15E, and 15C; (105) 10A, 10D, 10E, 10Q, 10Z, 10AA, 15E, and 15C; (106) 10A, 10D, 10P, 10N, 10AA, 15E, and 15C; (107) 10A, 10D, 10P, 10Y, 10Z, 10AA, 15E, and 15C; (108) 10A, 10B, 10M, 10AA, 15E, and 15C; (109) 10A, 10B, 10L, 10Z, 10AA, 15E, and 15C; (110) 10A, 10B, 10X, 10N, 10AA, 15E, and 15C; (111) 10A, 10B, 10X, 10Y, 10Z, 10AA, 15E, and 15C; (112) 10A, 10D, 10P, 10O, 15E, and 15C; (113) 10A, 10B, 10X, 10O, 15E, and 15C; (114) 10A, 10D, 10E, 10F, 10R, 10AA, 15E, and 15C; (115) 10A, 10D, 10E, 10F, 10G, 10S, 15E, and 15C; (116) 10A, 10B, 10C, 10AE, 10AB, 10Y, 10Z, 10AA, 15E, and 15C; (117) 10A, 10B, 10C, 10AE, 10AB, 10N, 10AA, 15E, and 15C; (118) 10A, 10B, 10C, 10AE, 10AB, 10O, 15E, and 15C; (119) 10AU, 10AB, 10Y, 10Z, 10AA, 15E, and 15C; (120) 10AU, 10AB, 10N, 10AA, 15E, and 15C; (121) 10AU, 10AB, 10O, 15E, and 15C; (122) 1T, 10AS, 10E, 10F, 10G, 10S, 15E, and 15C; (123) 1T, 10AS, 10I, 10G, 10S, 15E, and 15C; (124) 1T, 10AS, 10K, 10S, 15E, and 15C; (125) 1T, 10AS, 10I, 10R, 10AA, 15E, and 15C; (126) 1T, 10AS, 10E, 10F, 10R, 10AA, 15E, and 15C; (127) 1T, 10AS, 10E, 10Q, 10Z, 10AA, 15E, and 15C; (128) 1T, 10AS, 10P, 10N, 10AA, 15E, and 15C; (129) 1T, 10AS, 10P, 10Y, 10Z, 10AA, 15E, and 15C; (130) 1T, 10AS, 10P, 10O, 15E, and 15C; (131) 1T, 10AS, 10E, 10F, 10R, 10AA, 15E, and 15C; (132) 10AT, 10E, 10F, 10G, 10S, 15E, and 15C; (133) 10AT, 10I, 10G, 10S, 15E, and 15C; (134) 10AT, 10K, 10S, 15E, and 15C; (135) 10AT, 10I, 10R, 10AA, 15E, and 15C; (136) 10AT, 10E, 10F, 10R, 10AA, 15E, and 15C; (137) 10AT, 10E, 10Q, 10Z, 10AA, 15E, and 15C; (138) 10AT, 10P, 10N, 10AA, 15E, and 15C; (139) 10AT, 10P, 10Y, 10Z, 10AA, 15E, and 15C; (140) 10AT, 10P, 10O, 15E, and 15C; (141) 10AT, 10E, 10F, 10R, 10AA, 15E, and 15C; (142) 10A, 10D, 10E, 10F, 10G, 10S, 15A, and 15F; (143) 10A, 10D, 10I, 10G, 10S, 15A, and 15F; (144) 10A, 10D, 10K, 10S, 15A, and 15F; (145) 10A, 10H, 10F, 10G, 10S, 15A, and 15F; (146) 10A, 10J, 10G, 10S, 15A, and 15F; (147) 10A, 10J, 10R, 10AA, 15A, and 15F; (148) 10A, 10H, 10F, 10R, 10AA, 15A, and 15F; (149) 10A, 10H, 10Q, 10Z, 10AA, 15A, and 15F; (150) 10A, 10D, 10I, 10R, 10AA, 15A, and 15F; (151) 10A, 10D, 10E, 10F, 10R, 10AA, 15A, and 15F; (152) 10A, 10D, 10E, 10Q, 10Z, 10AA, 15A, and 15F; (153) 10A, 10D, 10P, 10N, 10AA, 15A, and 15F; (154) 10A, 10D, 10P, 10Y, 10Z, 10AA, 15A, and 15F; (155) 10A, 10B, 10M, 10AA, 15A, and 15F; (156) 10A, 10B, 10L, 10Z, 10AA, 15A, and 15F; (157) 10A, 10B, 10X, 10N, 10AA, 15A, and 15F; (158) 10A, 10B, 10X, 10Y, 10Z, 10AA, 15A, and 15F; (159) 10A, 10D, 10P, 10O, 15A, and 15F; (160) 10A, 10B, 10X, 10O, 15A, and 15F; (161) 10A, 10D, 10E, 10F, 10R, 10AA, 15A, and 15F; (162) 10A, 10D, 10E, 10F, 10G, 10S, 15A, and 15F; (163) 10A, 10B, 10C, 10AE, 10AB, 10Y, 10Z, 10AA, 15A, and 15F; (164) 10A, 10B, 10C, 10AE, 10AB, 10N, 10AA, 15A, and 15F; (165) 10A, 10B, 10C, 10AE, 10AB, 10O, 15A, and 15F; (166) 10AU, 10AB, 10Y, 10Z, 10AA, 15A, and 15F; (167) 10AU, 10AB, 10N, 10AA, 15A, and 15F; (168) 10AU, 10AB, 10O, 15A, and 15F; (169) 1T, 10AS, 10E, 10F, 10G, 10S, 15A, and 15F; (170) 1T, 10AS, 10I, 10G, 10S, 15A, and 15F; (171) 1T, 10AS, 10K, 10S, 15A, and 15F; (172) 1T, 10AS, 10I, 10R, 10AA, 15A, and 15F; (173) 1T, 10AS, 10E, 10F, 10R, 10AA, 15A, and 15F; (174) 1T, 10AS, 10E, 10Q, 10Z, 10AA, 15A, and 15F; (175) 1T, 10AS, 10P, 10N, 10AA, 15A, and 15F; (176) 1T, 10AS, 10P, 10Y, 10Z, 10AA, 15A, and 15F; (177) 1T, 10AS, 10P, 10O, 15A, and 15F; (178) 1T, 10AS, 10E, 10F, 10R, 10AA, 15A, and 15F; (179) 10AT, 10E, 10F, 10G, 10S, 15A, and 15F; (180) 10AT, 10I, 10G, 10S, 15A, and 15F; (181) 10AT, 10K, 10S, 15A, and 15F; (182) 10AT, 10I, 10R, 10AA, 15A, and 15F; (183) 10AT, 10E, 10F, 10R, 10AA, 15A, and 15F; (184) 10AT, 10E, 10Q, 10Z, 10AA, 15A, and 15F; (185) 10AT, 10P, 10N, 10AA, 15A, and 15F; (186) 10AT, 10P, 10Y, 10Z, 10AA, 15A, and 15F; (187) 10AT, 10P, 10O, 15A, and 15F; (188) 10AT, 10E, 10F, 10R, 10AA, 15A, and 15F; (189) 14A, 14B, 14C, 14D, 14E, and 13A; (190) 16A, 16B, 16C, and 16D; (191) 17A, 17B, 17C, and 17D; (192) 17A, 17E, 17F, and 17D; (193) 17A, 17B, 17C, 17H, 17I, and 17J; (194) 18A, 18B, 18C, 18D, and 18E; (195) 13A; and (196) 17A, 17E, 17F, 17H, 17I, and 17J,
wherein 1T is an acetyl-CoA carboxylase, wherein 10A is a 3-ketoacyl-ACP synthase, wherein 10B is an acetoacetyl-ACP reductase, wherein 10C is a 3-hydroxybutyryl-ACP dehydratase, wherein 10D is an acetoacetyl-CoA:ACP transferase, wherein 10E is an acetoacetyl-CoA hydrolase, transferase or synthetase, wherein 10F is an acetoacetate reductase (acid reducing), wherein 10G is a 3-oxobutyraldehyde reductase (aldehyde reducing), wherein 10H is an acetoacetyl-ACP thioesterase, wherein 10I is an acetoacetyl-CoA reductase (CoA-dependent, aldehyde forming), wherein 10J is an acetoacetyl-ACP reductase (aldehyde forming), wherein 10K is an acetoacetyl-CoA reductase (alcohol forming), wherein 10L is a 3-hydroxybutyryl-ACP thioesterase, wherein 10M is a 3-hydroxybutyryl-ACP reductase (aldehyde forming), wherein 10N is a 3-hydroxybutyryl-CoA reductase (aldehyde forming), wherein 10O is a 3-hydroxybutyryl-CoA reductase (alcohol forming), wherein 10P is an acetoacetyl-CoA reductase (ketone reducing), wherein 10Q is an acetoacetate reductase (ketone reducing), wherein 10R is a 3-oxobutyraldehyde reductase (ketone reducing), wherein 10S is a 4-hydroxy-2-butanone reductase, wherein 10X is a 3-hydroxybutyryl-CoA:ACP transferase, wherein 10Y is a 3-hydroxybutyryl-CoA hydrolase, transferase or synthetase, wherein 10Z is a 3-hydroxybutyrate reductase, wherein 10AA is a 3-hydroxybutyraldehyde reductase, wherein 10AB is a 3-hydroxybutyryl-CoA dehydratase, wherein 10AE is a crotonyl-CoA:ACP transferase, wherein 10AS is an acetoacetyl-CoA synthase, wherein 10AT is an acetyl-CoA:acetyl-CoA acyltransferase, wherein 10AU is a 4-hydroxybutyryl-CoA dehydratase, wherein 13A is a 2-butanol desaturase, wherein 14A is an acetolactate synthase, wherein 14B is an acetolactate decarboxylase, wherein 14C is a butanediol dehydrogenase, wherein 14D is a butanediol dehydratase, wherein 14E is a butanol dehydrogenase, wherein 15A is a 1,3-butanediol kinase, wherein 15B is a 3-hydroxybutyrylphosphate kinase, 15C is a 3-hydroxybutyryldiphosphate lyase, wherein 15D is a 1,3-butanediol diphosphokinase, wherein 15E is a 1,3-butanediol dehydratase, wherein 15F is a 3-hydroxybutyrylphosphate lyase, wherein 16A is a 3-oxopent-4-enoyl-CoA thiolase, wherein 16B is a 3-oxopent-4-enoyl-CoA hydrolase, synthetase or transferase, wherein 16C is a 3-oxopent-4-enoate decarboxylase or spontaneous, wherein 16D is a 3-buten-2-one reductase, wherein 17A is a 3-oxo-4-hydroxypentanoyl-CoA thiolase, wherein 17B is a 3-oxo-4-hydroxypentanoyl-CoA transferase, synthetase or hydrolase, wherein 17C is a 3-oxo-4-hydroxypentanoate reductase, wherein 17D is a 3,4-dihydroxypentanoate decarboxylase, wherein 17E is a 3-oxo-4-hydroxypentanoyl-CoA reductase, wherein 17F is a 3,4-dihydroxypentanoyl-CoA transferase, synthetase or hydrolase, wherein 17H is a 3,4-dihydroxypentanoate dehydratase, wherein 17I is a 4-oxopentanoate reductase, wherein 17J is a 4-hyd4-oxoperoxypentanoate decarboxylase, wherein 18A is a 3-oxoadipyl-CoA thiolase, wherein 18B is a 3-oxoadipyl-CoA transferase, synthetase or hydrolase, wherein 18C is a 3-oxoadipate decarboxylase or spontaneous, wherein 18D is a 4-oxopentanoate reductase, wherein 18E is a 4-hydroxypentanoate decarboxylase.

103-105. (canceled)

106. A method for producing 3-buten-2-ol, comprising culturing the non-naturally occurring microbial organism of claim 88 under conditions and for a sufficient period of time to produce 3-buten-2-ol.

107-108. (canceled)

109. Bioderived 3-buten-2-ol produced according to the method of claim 106.

110-122. (canceled)

123. A non-naturally occurring microbial organism having a formaldehyde fixation pathway and a methanol oxidation pathway, wherein said organism comprises at least one exogenous nucleic acid encoding a formaldehyde fixation pathway enzyme expressed in a sufficient amount to produce pyruvate, wherein said formaldehyde fixation pathway comprises:
(1) 1B and 1C; or (2) 1D,
wherein 1B is a 3-hexulose-6-phosphate synthase, wherein 1C is a 6-phospho-3-hexuloisomerase, wherein 1D is a dihydroxyacetone synthase,
wherein said methanol oxidation pathway comprises at least one exogenous nucleic acid encoding a methanol oxidation pathway enzyme expressed in a sufficient amount to produce formaldehyde in the presence of methanol, wherein said methanol oxidation pathway comprises 1A, wherein 1A a methanol dehydrogenase.

124-164. (canceled)

165. A method for producing butadiene comprising culturing the non-naturally occurring microbial organism of claim 28 under conditions and for a sufficient period of time to produce butadiene.

166. Bioderived butadiene produced according to the method of claim 165.

167. A method for producing 3-buten-2-ol, comprising culturing the non-naturally occurring microbial organism of claim 102 under conditions and for a sufficient period of time to produce 3-buten-2-ol.

168. Bioderived 3-buten-2-ol produced according to the method of claim 167.
